SEQUENCE LISTING (236 sequences)

SEQ ID NO: 1 - protein, 8 aa, Artificial Sequence
  source: /note=“Description of Artificial Sequence: Synthetic peptide”
GFTFSSYT

SEQ ID NO: 2 - protein, 8 aa, Artificial Sequence
  source: /note=“Description of Artificial Sequence: Synthetic peptide”
ISYDGNNK

SEQ ID NO: 3 - protein, 11 aa, Artificial Sequence
  source: /note=“Description of Artificial Sequence: Synthetic peptide”
ARTGWLGPFDY

SEQ ID NO: 4 - protein, 7 aa, Artificial Sequence
  source: /note=“Description of Artificial Sequence: Synthetic peptide”
QSVGSSY

SEQ ID NO: 5 - protein, 3 aa, Artificial Sequence
  source: /note=“Description of Artificial Sequence: Synthetic peptide”
SSY

SEQ ID NO: 6 - protein, 9 aa, Artificial Sequence
  source: /note=“Description of Artificial Sequence: Synthetic peptide”
QQYGSSPWT

SEQ ID NO: 7 - protein, 8 aa, Artificial Sequence
  source: /note=“Description of Artificial Sequence: Synthetic peptide”
GITFSNSG

SEQ ID NO: 8 - protein, 8 aa, Artificial Sequence
  source: /note=“Description of Artificial Sequence: Synthetic peptide”
IWYDGSKR

SEQ ID NO: 9 - protein, 6 aa, Artificial Sequence
  source: /note=“Description of Artificial Sequence: Synthetic peptide”
ATNDDY

SEQ ID NO: 10 - protein, 7 aa, Artificial Sequence
  source: /note=“Description of Artificial Sequence: Synthetic peptide”
QSVSSYL

SEQ ID NO: 11 - protein, 3 aa, Artificial Sequence
  source: /note=“Description of Artificial Sequence: Synthetic peptide”
DAS

SEQ ID NO: 12 - protein, 9 aa, Artificial Sequence
  source: /note=“Description of Artificial Sequence: Synthetic peptide”
QQSSNWPRT

SEQ ID NO: 13 - protein, 8 aa, Artificial Sequence
  source: /note=“Description of Artificial Sequence: Synthetic peptide”
GYTFTNYY

SEQ ID NO: 14 - protein, 8 aa, Artificial Sequence
  source: /note=“Description of Artificial Sequence: Synthetic peptide”
INPSNGGT

SEQ ID NO: 15 - protein, 13 aa, Artificial Sequence
  source: /note=“Description of Artificial Sequence: Synthetic peptide”
ARRDYRFDMGFDY

SEQ ID NO: 16 - protein, 10 aa, Artificial Sequence
  source: /note=“Description of Artificial Sequence: Synthetic peptide”
KGVSTSGYSY

SEQ ID NO: 17 - protein, 3 aa, Artificial Sequence
  source: /note=“Description of Artificial Sequence: Synthetic peptide”
LAS

SEQ ID NO: 18 - protein, 9 aa, Artificial Sequence
  source: /note=“Description of Artificial Sequence: Synthetic peptide”
QHSRDLPLT

SEQ ID NO: 19 - protein, 8 aa, Artificial Sequence
  source: /note=“Description of Artificial Sequence: Synthetic peptide”
GYTFTDSY

SEQ ID NO: 20 - protein, 8 aa, Artificial Sequence
  source: /note=“Description of Artificial Sequence: Synthetic peptide”
MYPDNGDS

SEQ ID NO: 21 - protein, 10 aa, Artificial Sequence
  source: /note=“Description of Artificial Sequence: Synthetic peptide”
VLAPRWYFSV

SEQ ID NO: 22 - protein, 6 aa, Artificial Sequence
  source: /note=“Description of Artificial Sequence: Synthetic peptide”
QDISNY

SEQ ID NO: 23 - protein, 3 aa, Artificial Sequence
  source: /note=“Description of Artificial Sequence: Synthetic peptide”
YTS

SEQ ID NO: 24 - protein, 9 aa, Artificial Sequence
  source: /note=“Description of Artificial Sequence: Synthetic peptide”
QQGHTLPPT

SEQ ID NO: 25 - protein, 8 aa, Artificial Sequence
  source: /note=“Description of Artificial Sequence: Synthetic peptide”
GFTFSSYD

SEQ ID NO: 26 - protein, 8 aa, Artificial Sequence
  source: /note=“Description of Artificial Sequence: Synthetic peptide”
IWYDGSNK

SEQ ID NO: 27 - protein, 12 aa, Artificial Sequence
  source: /note=“Description of Artificial Sequence: Synthetic peptide”
ARGSGNWGFFDY

SEQ ID NO: 28 - protein, 6 aa, Artificial Sequence
  source: /note=“Description of Artificial Sequence: Synthetic peptide”
QGISRW

SEQ ID NO: 29 - protein, 3 aa, Artificial Sequence
  source: /note=“Description of Artificial Sequence: Synthetic peptide”
AAS

SEQ ID NO: 30 - protein, 9 aa, Artificial Sequence
  source: /note=“Description of Artificial Sequence: Synthetic peptide”
QQYNTYPRT

SEQ ID NO: 31 - protein, 8 aa, Artificial Sequence
  source: /note=“Description of Artificial Sequence: Synthetic peptide”
GYTFTSYR

SEQ ID NO: 32 - protein, 8 aa, Artificial Sequence
  source: /note=“Description of Artificial Sequence: Synthetic peptide”
INPSTGYT

SEQ ID NO: 33 - protein, 9 aa, Artificial Sequence
  source: /note=“Description of Artificial Sequence: Synthetic peptide”
ARGGGVFDY

SEQ ID NO: 34 - protein, 6 aa, Artificial Sequence
  source: /note=“Description of Artificial Sequence: Synthetic peptide”
SSSISY

SEQ ID NO: 35 - protein, 3 aa, Artificial Sequence
  source: /note=“Description of Artificial Sequence: Synthetic peptide”
TTS

SEQ ID NO: 36 - protein, 9 aa, Artificial Sequence
  source: /note=“Description of Artificial Sequence: Synthetic peptide”
HQRSTYPLT

SEQ ID NO: 37 - protein, 5 aa, Artificial Sequence
  source: /note=“Description of Artificial Sequence: Synthetic peptide”
SYGMH

SEQ ID NO: 38 - protein, 17 aa, Artificial Sequence
  source: /note=“Description of Artificial Sequence: Synthetic peptide”
VIWYEGSNKYYADSVKG

SEQ ID NO: 39 - protein, 15 aa, Artificial Sequence
  source: /note=“Description of Artificial Sequence: Synthetic peptide”
GGSMVRGDYYYGMDV

SEQ ID NO: 40 - protein, 11 aa, Artificial Sequence
  source: /note=“Description of Artificial Sequence: Synthetic peptide”
RASQGISSALA

SEQ ID NO: 41 - protein, 7 aa, Artificial Sequence
  source: /note=“Description of Artificial Sequence: Synthetic peptide”
DASSLES

SEQ ID NO: 42 - protein, 9 aa, Artificial Sequence
  source: /note=“Description of Artificial Sequence: Synthetic peptide”
QQFNSYPYT

SEQ ID NO: 43 - protein, 5 aa, Artificial Sequence
  source: /note=“Description of Artificial Sequence: Synthetic peptide”
DYYWN

SEQ ID NO: 44 - protein, 16 aa, Artificial Sequence
  source: /note=“Description of Artificial Sequence: Synthetic peptide”
EINHRGSTNSNPSLKS

SEQ ID NO: 45 - protein, 12 aa, Artificial Sequence
  source: /note=“Description of Artificial Sequence: Synthetic peptide”
GYSDYEYNWFDP

SEQ ID NO: 46 - protein, 11 aa, Artificial Sequence
  source: /note=“Description of Artificial Sequence: Synthetic peptide”
RASQSISSYLA

SEQ ID NO: 47 - protein, 7 aa, Artificial Sequence
  source: /note=“Description of Artificial Sequence: Synthetic peptide”
DASNRAT

SEQ ID NO: 48 - protein, 9 aa, Artificial Sequence
  source: /note=“Description of Artificial Sequence: Synthetic peptide”
QQRSNWPLT

SEQ ID NO: 49 - protein, 5 aa, Artificial Sequence
  source: /note=“Description of Artificial Sequence: Synthetic peptide”
  MOD_RES (3)..(3): Any amino acid
LPXTG

SEQ ID NO: 50 - protein, 8 aa, Artificial Sequence
  source: /note=“Description of Artificial Sequence: Synthetic peptide”
GYSFSTYW

SEQ ID NO: 51 - protein, 8 aa, Artificial Sequence
  source: /note=“Description of Artificial Sequence: Synthetic peptide”
IYPGDSYT

SEQ ID NO: 52 - protein, 9 aa, Artificial Sequence
  source: /note=“Description of Artificial Sequence: Synthetic peptide”
ARGYGIFDY

SEQ ID NO: 53 - protein, 6 aa, Artificial Sequence
  source: /note=“Description of Artificial Sequence: Synthetic peptide”
NIGDQY

SEQ ID NO: 54 - protein, 3 aa, Artificial Sequence
  source: /note=“Description of Artificial Sequence: Synthetic peptide”
QDK

SEQ ID NO: 55 - protein, 11 aa, Artificial Sequence
  source: /note=“Description of Artificial Sequence: Synthetic peptide”
ATYTGFGSLAV

SEQ ID NO: 56 - protein, 8 aa, Artificial Sequence
  source: /note=“Description of Artificial Sequence: Synthetic peptide”
GYTFTDYN

SEQ ID NO: 57 - protein, 8 aa, Artificial Sequence
  source: /note=“Description of Artificial Sequence: Synthetic peptide”
INPNYEST

SEQ ID NO: 58 - protein, 9 aa, Artificial Sequence
  source: /note=“Description of Artificial Sequence: Synthetic peptide”
RDKGWYFDV

SEQ ID NO: 59 - protein, 5 aa, Artificial Sequence
  source: /note=“Description of Artificial Sequence: Synthetic peptide”
SSVKN

SEQ ID NO: 60 - protein, 3 aa, Artificial Sequence
  source: /note=“Description of Artificial Sequence: Synthetic peptide”
YTS

SEQ ID NO: 61 - protein, 9 aa, Artificial Sequence
  source: /note=“Description of Artificial Sequence: Synthetic peptide”
QQFTSSPYT

SEQ ID NO: 62 - protein, 10 aa, Artificial Sequence
  source: /note=“Description of Artificial Sequence: Synthetic peptide”
GFSLSTSGMG

SEQ ID NO: 63 - protein, 7 aa, Artificial Sequence
  source: /note=“Description of Artificial Sequence: Synthetic peptide”
IWWDDDK

SEQ ID NO: 64 - protein, 11 aa, Artificial Sequence
  source: /note=“Description of Artificial Sequence: Synthetic peptide”
ARLTGTRYFDY

SEQ ID NO: 65 - protein, 6 aa, Artificial Sequence
  source: /note=“Description of Artificial Sequence: Synthetic peptide”
QDINKF

SEQ ID NO: 66 - protein, 3 aa, Artificial Sequence
  source: /note=“Description of Artificial Sequence: Synthetic peptide”
YTS

SEQ ID NO: 67 - protein, 8 aa, Artificial Sequence
  source: /note=“Description of Artificial Sequence: Synthetic peptide”
LQYGNLWT

SEQ ID NO: 68 - protein, 8 aa, Artificial Sequence
  source: /note=“Description of Artificial Sequence: Synthetic peptide”
GYTFTDYS

SEQ ID NO: 69 - protein, 8 aa, Artificial Sequence
  source: /note=“Description of Artificial Sequence: Synthetic peptide”
INTETGEP

SEQ ID NO: 70 - protein, 12 aa, Artificial Sequence
  source: /note=“Description of Artificial Sequence: Synthetic peptide”
ATYYGSSYVPDY

SEQ ID NO: 71 - protein, 6 aa, Artificial Sequence
  source: /note=“Description of Artificial Sequence: Synthetic peptide”
QNVGTA

SEQ ID NO: 72 - protein, 3 aa, Artificial Sequence
  source: /note=“Description of Artificial Sequence: Synthetic peptide”
WTS

SEQ ID NO: 73 - protein, 8 aa, Artificial Sequence
  source: /note=“Description of Artificial Sequence: Synthetic peptide”
QYSDYPYT

SEQ ID NO: 74 - protein, 7 aa, Artificial Sequence
  source: /note=“Description of Artificial Sequence: Synthetic peptide”
GYTFTDY

SEQ ID NO: 75 - protein, 8 aa, Artificial Sequence
  source: /note=“Description of Artificial Sequence: Synthetic peptide”
WVDPEYGS

SEQ ID NO: 76 - protein, 12 aa, Artificial Sequence
  source: /note=“Description of Artificial Sequence: Synthetic peptide”
ARDDGSYSPFDY

SEQ ID NO: 77 - protein, 6 aa, Artificial Sequence
  source: /note=“Description of Artificial Sequence: Synthetic peptide”
QNINKY

SEQ ID NO: 78 - protein, 3 aa, Artificial Sequence
  source: /note=“Description of Artificial Sequence: Synthetic peptide”
YTS

SEQ ID NO: 79 - protein, 8 aa, Artificial Sequence
  source: /note=“Description of Artificial Sequence: Synthetic peptide”
LQYVNLLT

SEQ ID NO: 80 - protein, 6 aa, Artificial Sequence
  source: /note=“Description of Artificial Sequence: Synthetic peptide”
ENVVTY

SEQ ID NO: 81 - protein, 3 aa, Artificial Sequence
  source: /note=“Description of Artificial Sequence: Synthetic peptide”
GAS

SEQ ID NO: 82 - protein, 8 aa, Artificial Sequence
  source: /note=“Description of Artificial Sequence: Synthetic peptide”
QGYSYPYT

SEQ ID NO: 83 - protein, 118 aa, Artificial Sequence
  source: /note=“Description of Artificial Sequence: Synthetic polypeptide”
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 84 - protein, 108 aa, Artificial Sequence
  source: /note=“Description of Artificial Sequence: Synthetic polypeptide”
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 85 - protein, 113 aa, Artificial Sequence [sequence omitted]

SEQ ID NO: 86 - protein, 107 aa, Artificial Sequence [sequence omitted]

SEQ ID NO: 87 - protein, 120 aa, Artificial Sequence [sequence omitted]

SEQ ID NO: 88 - protein, 111 aa, Artificial Sequence [sequence omitted]

SEQ ID NO: 89 - protein, 118 aa, Artificial Sequence [sequence omitted]

SEQ ID NO: 90 - protein, 107 aa, Artificial Sequence [sequence omitted]

SEQ ID NO: 91 - protein, 121 aa, Artificial Sequence
  source: /note=“Description of Artificial Sequence: Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 92 - protein, 108 aa, Artificial Sequence
  source: /note=“Description of Artificial Sequence: Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 93 - protein, 123 aa, Artificial Sequence
  source: /note=“Description of Artificial Sequence: Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 94 - protein, 106 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 95 - protein, 117 aa, Artificial Sequence
  source: /note=“Description of Artificial Sequence: Synthetic polypeptide”
EVQLQQSGAELVKPGASVKISCKASGYTFTDYNMDWVKQSHGKSLEWIGDINPNYESTSYNQKFKGKATLTVDKSSSTAYMEVRSLTSEDTAVFYCARDKGWYFDVWGAGTTVTVSS

SEQ ID NO: 96 - protein, 106 aa, Artificial Sequence
  source: /note=“Description of Artificial Sequence: Synthetic polypeptide”
ENVLTQSPAIMSASLGEKVTMSCRASSSVKNMYWYQQKSDASPKLWIYYTSNLAPGVPARFSGSGSGNSYSLTISSMEGEDAATYYCQQFTSSPYTFGGGTKLELK

SEQ ID NO: 97 - protein, 119 aa, Artificial Sequence
  source: /note=“Description of Artificial Sequence: Synthetic polypeptide”
QVTLKESGPGILQPSQTLSLTCSFSGFSLSTSGMGVGWIRQPSGKGLEWLAHIWWDDDKFYNPSLKSQLTISKDTSRNQVFLKLTSVVTADTATYYCARLTGTRYFDYWGQGTTLTVSS

SEQ ID NO: 98 - protein, 106 aa, Artificial Sequence
  source: /note=“Description of Artificial Sequence: Synthetic polypeptide”
DVQMTQSPSSLSASLGGKVTITCKASQDINKFIAWYQHKPGKGPRLLIHYTSTLQPGIPSKFSGSGSGRDYSFSISNLEPEDIATYYCLQYGNLWTFGGGTKLEIT

SEQ ID NO: 99 - protein, 119 aa, Artificial Sequence
  source: /note=“Description of Artificial Sequence: Synthetic polypeptide”
QIQLVQSGPELKKPGETVKISCKASGYTFTDYSMHWVKQAPGKGLKWMGWINTETGEPTYADDFKGRFAFSSETSTSTAYLQINNLKNDDTTTYFCATYYGSSYVPDYWGQGTSLTVSS

SEQ ID NO: 100 - protein, 107 aa, Artificial Sequence
  source: /note=“Description of Artificial Sequence: Synthetic polypeptide”
DIVMTQSHKFMSTSVGDRVSITCKASQNVGTAVAWYQHKPGQSPKLLIYWTSSRHTGVPDRFTGSGSGTEFTLTISNVQSEDLADYFCHQYSDYPYTFGGGTKLEIK

SEQ ID NO: 101 - protein, 119 aa, Artificial Sequence
  source: /note=“Description of Artificial Sequence: Synthetic polypeptide”
EVQLQQSGPEVGRPGSSVKISCKASGYTFTDYIMHWVKQSPGQGLEWIGWVDPEYGSTDYAEKFKKKATLTADTSSNTAYIQLSSLTSEDTATYFCARDDGSYSPFDYWGQGVMVTVSS

SEQ ID NO: 102 - protein, 106 aa, Artificial Sequence
  source: /note=“Description of Artificial Sequence: Synthetic polypeptide”
DIQMTQSPPSLSASLGDKVTITCQASQNINKYIAWYQQKPGKAPRLLIRYTSTLESGTPSRFSGSGSGRDYSFSISNVESEDIASYYCLQYVNLLTFGAGTKLEIK

SEQ ID NO: 103 - protein, 107 aa, Artificial Sequence
  source: /note=“Description of Artificial Sequence: Synthetic polypeptide”
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 104 - protein, 120 aa, Artificial Sequence
  source: /note=“Description of Artificial Sequence: Synthetic polypeptide”
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 105 - protein, 109 aa, Artificial Sequence
  source: /note=“Description of Artificial Sequence: Synthetic polypeptide”
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 106 - protein, 117 aa, Artificial Sequence
  source: /note=“Description of Artificial Sequence: Synthetic polypeptide”
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 107 - protein, 107 aa, Artificial Sequence
  source: /note=“Description of Artificial Sequence: Synthetic polypeptide”
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 108 - protein, 107 aa, Artificial Sequence
  source: /note=“Description of Artificial Sequence: Synthetic polypeptide”
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIK

SEQ ID NO: 109 - protein, 119 aa, Artificial Sequence
  source: /note=“Description of Artificial Sequence: Synthetic polypeptide”
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSS

SEQ ID NO: 110 - protein, 119 aa, Artificial Sequence
  source: /note=“Description of Artificial Sequence: Synthetic polypeptide”
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVSS

SEQ ID NO: 111 - protein, 120 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 112 - protein, 107 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 113 - protein, 140 aa, Artificial Sequence
  source: /note=“Description of Artificial Sequence: Synthetic polypeptide”
MAVLALLFCLVTFPSCILSQVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 114 - protein, 121 aa, Artificial Sequence
  source: /note=“Description of Artificial Sequence: Synthetic polypeptide”
QVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 115 - protein, 127 aa, Artificial Sequence
  source: /note=“Description of Artificial Sequence: Synthetic polypeptide”
MKFPSQLLLFLLFRITGIICDIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 116 - protein, 107 aa, Artificial Sequence
  source: /note=“Description of Artificial Sequence: Synthetic polypeptide”
DIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 117 - protein, 126 aa, Artificial Sequence
  source: /note=“Description of Artificial Sequence: Synthetic polypeptide”
EVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSS

SEQ ID NO: 118 - protein, 107 aa, Artificial Sequence
  source: /note=“Description of Artificial Sequence: Synthetic polypeptide”
DIQMTQSPTSLSASLGDRVTITCQASQSISSYLAWYQQKPGQAPKLLIYGASRLQTGVPSRFSGSGSGTSFTLTISGLEAEDAGTYYCQQYDSLPVTFGQGTKVELK

SEQ ID NO: 119 - protein, 107 aa, Artificial Sequence
  source: /note=“Description of Artificial Sequence: Synthetic polypeptide”
DIQMTQSPSSLSASLGDRVTITCQASQSIVSYLAWYQQKPGQAPKLLIYGASRLQTGVPSRFSGSGSGTSFTLTISGLEAEDAGTYYCQQYASAPVTFGQGTGVELK

SEQ ID NO: 120 - protein, 107 aa, Artificial Sequence
  source: /note=“Description of Artificial Sequence: Synthetic polypeptide”
DIQMTQSPSSLSASLGDRVTITCQASQSISSYLAWYQQKPGQAPKLLIYGTSRLKTGVPSRFSGSGSGTSFTLTISGLEAEDAGTYYCQQYYSAPVTFGQGTKVELK

SEQ ID NO: 121 - protein, 107 aa, Artificial Sequence
  source: /note=“Description of Artificial Sequence: Synthetic polypeptide”
DIQMTQSPSSLSPSLGDRVTITCQASQTISSFLAWYHQKPGQPPKLLIYRASIPQTGVPSRFSGSGSGTSFTLTIGGLEAEDAGTYYCQQYVSAPPTFGQGTKVELK

SEQ ID NO: 122 - protein, 107 aa, Artificial Sequence
  source: /note=“Description of Artificial Sequence: Synthetic polypeptide”
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 123 - protein, 121 aa, Artificial Sequence [sequence omitted]

SEQ ID NO: 124 - protein, 109 aa, Artificial Sequence [sequence omitted]

SEQ ID NO: 125 - protein, 114 aa, Artificial Sequence
  source: /note=“Description of Artificial Sequence: Synthetic polypeptide”
EVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSS

SEQ ID NO: 126 - protein, 113 aa, Artificial Sequence
  source: /note=“Description of Artificial Sequence: Synthetic polypeptide”
DIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIK

SEQ ID NO: 127 - protein, 116 aa, Artificial Sequence
  source: /note=“Description of Artificial Sequence: Synthetic polypeptide”
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 128 - protein, 108 aa, Artificial Sequence
  source: /note=“Description of Artificial Sequence: Synthetic polypeptide”
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 129 - protein, 107 aa, Artificial Sequence
  source: /note=“Description of Artificial Sequence: Synthetic polypeptide”
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 130 - protein, 125 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 131 - protein, 117 aa, Artificial Sequence
  source: /note=“Description of Artificial Sequence: Synthetic polypeptide”
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 132 - protein, 111 aa, Artificial Sequence
  source: /note=“Description of Artificial Sequence: Synthetic polypeptide”
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 133 - protein, 118 aa, Artificial Sequence
  source: /note=“Description of Artificial Sequence: Synthetic polypeptide”
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 134 - protein, 111 aa, Artificial Sequence
  source: /note=“Description of Artificial Sequence: Synthetic polypeptide”
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 135 - protein, 122 aa, Artificial Sequence
  source: /note=“Description of Artificial Sequence: Synthetic polypeptide”
QLQLQESGPGLVKPSETLSLTCTVSGGSITSSSYYWGWIRQPPGKGLEWIGSIYYRGSTNYNPSLKSRVTISVDSSKNQFYLKVSSVTAVDTAVYYCARQNGAARPSWFDPWGQGTLVTVSS

SEQ ID NO: 136 - protein, 108 aa, Artificial Sequence
  source: /note=“Description of Artificial Sequence: Synthetic polypeptide”
EIVLTQSPDTISLSPGERATLSCRASQSISSIYLAWYQQKPGQAPRLLIYGASSRVTGIPDRFSVSGSGTDFTLTISRLEPEDFAVYYCQHYGISPFTFGPGTKVDIR

SEQ ID NO: 137 - protein, 117 aa, Artificial Sequence
  source: /note=“Description of Artificial Sequence: Synthetic polypeptide”
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 138 - protein, 110 aa, Artificial Sequence
  source: /note=“Description of Artificial Sequence: Synthetic polypeptide”
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 139 - protein, 107 aa, Artificial Sequence
  source: /note=“Description of Artificial Sequence: Synthetic polypeptide”
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 140 - protein, 121 aa, Artificial Sequence
  source: /note=“Description of Artificial Sequence: Synthetic polypeptide”
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSS

SEQ ID NO: 141 - protein, 122 aa, Artificial Sequence [sequence omitted]

SEQ ID NO: 142 - protein, 107 aa, Artificial Sequence [sequence omitted]

SEQ ID NO: 143 - protein, 117 aa, Artificial Sequence
  source: /note=“Description of Artificial Sequence: Synthetic polypeptide”
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHLHGSFASWGQGTTVTVSS

SEQ ID NO: 144 - protein, 107 aa, Artificial Sequence
  source: /note=“Description of Artificial Sequence: Synthetic polypeptide”
EIVLTQSPATLSLSPGERATLSCQASQSISNFLHWYQQRPGQAPRLLIRYRSQSISGIPARFSGSGSGTDFTLTISSLEPEDFAVYYCQQSGSWPLTFGGGTKVEIK

SEQ ID NO: 145 - protein, 119 aa, Artificial Sequence
  source: /note=“Description of Artificial Sequence: Synthetic polypeptide”
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 146 - protein, 108 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 147 - protein, 116 aa, Artificial Sequence
  source: /note=“Description of Artificial Sequence: Synthetic polypeptide”
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCASLITTEDYWGQGTTVTVSS

SEQ ID NO: 148 - protein, 108 aa, Artificial Sequence
  source: /note=“Description of Artificial Sequence: Synthetic polypeptide”
EIVLTQSPSSLSLSPGERVTITCKASQDINSYLSWYQQKPGKAPKLLIYYANRLVDGVPARFSGSGSGQDYTLTISSLEPEDFAVYYCLQYDEFPYTFGGGTKLEIKR

SEQ ID NO: 149 - protein, 124 aa, Artificial Sequence
  source: /note=“Description of Artificial Sequence: Synthetic polypeptide”
QVQLQESGPGLVRPSQTLSLTCTASGYTFTDYVIHWVKQPPGRGLEWIGYINPYDDDTTYNQKFKGRVTMLVDTSSNTAYLRLSSVTAEDTAVYYCARRGNSYDGYFDYSMDYWGSGTPVTVSS

SEQ ID NO: 150 - protein, 108 aa, Artificial Sequence
  source: /note=“Description of Artificial Sequence: Synthetic polypeptide”
DIQMTQSPSSLSASVGDRVTITCRASQNVGTAVAWLQQTPGKAPKLLIYSASNRYTGVPSRFSGSGSGTDYTFTISSLQPEDIATYYCQQYTNYPMYTFGQGTKVQIK

SEQ ID NO: 151 - protein, 121 aa, Artificial Sequence
  source: /note=“Description of Artificial Sequence: Synthetic polypeptide”
QVQLQESGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVSS

SEQ ID NO: 152 - protein, 120 aa, Artificial Sequence
  source: /note=“Description of Artificial Sequence: Synthetic polypeptide”
QVQLQQSGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVS

SEQ ID NO: 153 - protein, 115 aa, Artificial Sequence
  source: /note=“Description of Artificial Sequence: Synthetic polypeptide”
QAVLTQPSSLSASPGASASLTCTLPSGINVGTYRIFWFQQKPGSPPQYLLSYKSDSDNHQGSGVPSRFSGSKDASANAGILLISGLQSEDEADYYCMIWHSSAWVFGGGTKLTVL

SEQ ID NO: 154 - protein, 122 aa, Artificial Sequence
  source: /note=“Description of Artificial Sequence: Synthetic polypeptide”
QVQLVQSGAEVKKPGASVKVSCKTSGYTFTDYYIHWVRQAPGQGLEWMGWINPNRGGTNYAQKFQGRVTMTRDTSIATAYMELSRLRSDDTAVYYCATASLKIAAVGTFDCWGQGTLVTVSS

SEQ ID NO: 155 - protein, 107 aa, Artificial Sequence
  source: /note=“Description of Artificial Sequence: Synthetic polypeptide”
SYELTQPPSVSVSPGQTARITCSGDALSKQYAFWFQQKPGQAPILVIYQDTKRPSGIPGRFSGSSSGTTVTLTISGAQADDEADYYCQSADSSGTYVFGTGTKVTVL

SEQ ID NO: 156 - protein, 116 aa, Artificial Sequence
  source: /note=“Description of Artificial Sequence: Synthetic polypeptide”
EVQLVETGGGVVQPGRSLRLSCAASGFSFSTHGMYWVRQPPGKGLEWVAVISYDGSDKKYADSVKGRFTISRDNSKNTVYLEMSSVRAEDTALYYCFCRRDAFDLWGQGTMVTVSS

SEQ ID NO: 157 - protein, 108 aa, Artificial Sequence
  source: /note=“Description of Artificial Sequence: Synthetic polypeptide”
SYVLTQPPSVSVSPGQTARITCSGDALPKKYAYWYQQKSGQAPVLVIYEDTKRPSGIPERFSGSSSGTMATLTISGAQVEDEADYYCYSTDSSGNYWVFGGGTEVTVL

SEQ ID NO: 158 - protein, 124 aa, Artificial Sequence
  source: /note=“Description of Artificial Sequence: Synthetic polypeptide”
EVQLVESGGGLVEPGGSLRLSCAASGFTFSDAWMNWVRQAPGKGLEWVGRIKTKSDGGTTDYAAPVRGRFSISRDDSKNTLFLEMNSLKTEDTAIYYCFITVIVVSSESPLDHWGQGTLVTVSS

SEQ ID NO: 159 - protein, 108 aa, Artificial Sequence
  source: /note=“Description of Artificial Sequence: Synthetic polypeptide”
SYELTQPPSVSVSPGQTARITCSGDELPKQYAYWYQQKPGQAPVLVIYKDRQRPSGIPERFSGSSSGTTVTLTISGVQAEDEADYYCQSAYSINTYVIFGGGTKLTVL

SEQ ID NO: 160 - protein, 125 aa, Artificial Sequence
  source: /note=“Description of Artificial Sequence: Synthetic polypeptide”
EVQLVESGGGLVKPGGSLRLSCAASGFTFSDYYMSWIRQAPGKGLEWISYISSGSSYTNYADSVKGRFTISRDNAKKSVYLEVNGLTVEDTAVYYCARVRYGDREMATIGGFDFWGQGTLVTVSS

SEQ ID NO: 161 - protein, 108 aa, Artificial Sequence
  source: /note=“Description of Artificial Sequence: Synthetic polypeptide”
SYELTQPPSVSVSPGQTARITCSGDALPKQYAYWYQQSPGQAPVLVIYKDSERPSGIPERFSGSSSGTTVTLTISGVQAEDEADYYCQSADSGGTSRIFGGGTKLTVL

SEQ ID NO: 162 - protein, 124 aa, Artificial Sequence
  source: /note=“Description of Artificial Sequence: Synthetic polypeptide”
QVQLQESGPGLVRSTETLSLTCLVSGDSINSHYWSWLRQSPGRGLEWIGYIYYTGPTNYNPSLKSRVSISLGTSKDQFSLKLSSVTAADTARYYCARNKVFWRGSDFYYYMDVWGKGTTVTVSS

SEQ ID NO: 163 - protein, 110 aa, Artificial Sequence
  source: /note=“Description of Artificial Sequence: Synthetic polypeptide”
EIVLTQSPGTLSLSLGERATLSCRASQSLANNYLAWYQQKPGQAPRLLMYDASTRATGIPDRFSGSGSGTDFTLTISRLEPEDFAVYYCQQFVTSHHMYIFGQGTKVEIK

SEQ ID NO: 164 - protein, 129 aa, Artificial Sequence
  source: /note=“Description of Artificial Sequence: Synthetic polypeptide”
HVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 165 - protein, 129 aa, Artificial Sequence
  source: /note=“Description of Artificial Sequence: Synthetic polypeptide”
QVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 166 - protein, 121 aa, Artificial Sequence
  source: /note=“Description of Artificial Sequence: Synthetic polypeptide”
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDWSRSGYYLPDYWGQGTLVTVSS

SEQ ID NO: 167 - protein, 107 aa, Artificial Sequence
  source: /note=“Description of Artificial Sequence: Synthetic polypeptide”
ETTLTQSPGTLSLSPGERATLSCRASQTVTRNYLAWYQQKPGQAPRLLMYGASNRAAGVPDRFSGSGSGTDFTLTISRLEPEDFAVYYCQQFGSPYTFGQGTKVEIK

SEQ ID NO: 168 - protein, 112 aa, Artificial Sequence
  source: /note=“Description of Artificial Sequence: Synthetic polypeptide”
DVVMTQSPLSLPVTLGQPASISCRSSQSLLHSNGYNYLDWYLQRPGQSPHLLIFLGSNRASGVPDRFSGSGSGTDFTLKISRVEAEDVGIYYCMQALQTPPTFGQGTKVEIK

SEQ ID NO: 169-170 - skipped (2 entries)

SEQ ID NO: 171 - protein, 119 aa, Artificial Sequence; identical to SEQ ID NO: 145
  source: /note=“Description of Artificial Sequence: Synthetic polypeptide”

SEQ ID NO: 172 - protein, 108 aa, Artificial Sequence; identical to SEQ ID NO: 146
  source: /note=“Description of Artificial Sequence: Synthetic polypeptide”

SEQ ID NO: 173 - protein, 107 aa, Artificial Sequence [sequence omitted]

SEQ ID NO: 174 - protein, 118 aa, Artificial Sequence [sequence omitted]

SEQ ID NO: 175 - protein, 124 aa, Artificial Sequence
  source: /note=“Description of Artificial Sequence: Synthetic polypeptide”
QVQLQESGPGLVKPSQTLSLTCTVSGGSISSGVYYWTWIRQHPGNGLEWIGYIYYSGSTSYNPSLKSRVTISVDTSKKQFSLNLTSVTAADTAVYYCAREGPLRGDYYYGLDVWGQGTTVTVSS

SEQ ID NO: 176 - protein, 108 aa, Artificial Sequence
  source: /note=“Description of Artificial Sequence: Synthetic polypeptide”
EIVLTQSPGTLSLSPGERATLSCRAGQTISSRYLAWYQQKPGQAPRPLIYGASSRATGIPDRFSGSGSGTDFTLTISRLEPEDFAVYYCQQYGSSPRTFGQGTKVEIK

SEQ ID NO: 177 - protein, 128 aa, Artificial Sequence
  source: /note=“Description of Artificial Sequence: Synthetic polypeptide”
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAMYYCARYRGPAAGRGDFYYFGMDVWGQGTTVTVSS

SEQ ID NO: 178 - protein, 113 aa, Artificial Sequence
  source: /note=“Description of Artificial Sequence: Synthetic polypeptide”
DIVMTQTPLSLSVTPGQPASIFCKSSQSLLNSDGKTYLCWYLQKPGQPPQLLIYEVSNRFSGVPDRFSGSGSGTDFTLKISRVEAEDVGVYYCMQGIQLPWAFFGQGTKVEIK

SEQ ID NO: 179 - protein, 125 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYGGSNKYYADSVKGRFTISRDNSKNTLYLQMNSLRAEDTAVYYCARDLAARRGDYYYYGMDVWGQGTTVTVSS

SEQ ID NO: 180 - protein, 107 aa, Artificial Sequence
  source: /note=“Description of Artificial Sequence: Synthetic polypeptide”
SSELTQDPVVSVALGQTVRITCQGDSLRSYYLSWYQQKPGQAPVLVIYGKNNRPSGIPDRFSGSNSGNTASLTITGAQAEDEADYYCNSRDSSGNHLFGGGTKLTVL

SEQ ID NO: 181 - protein, 125 aa, Artificial Sequence
  source: /note=“Description of Artificial Sequence: Synthetic polypeptide”
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSS

SEQ ID NO: 182 - protein, 106 aa, Artificial Sequence
  source: /note=“Description of Artificial Sequence: Synthetic polypeptide”
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVL

SEQ ID NO: 183 - protein, 119 aa, Artificial Sequence
  source: /note=“Description of Artificial Sequence: Synthetic polypeptide”
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 184 - protein, 107 aa, Artificial Sequence
  source: /note=“Description of Artificial Sequence: Synthetic polypeptide”
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 185 - protein, 116 aa, Artificial Sequence
  source: /note=“Description of Artificial Sequence: Synthetic polypeptide”
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 186 - protein, 106 aa, Artificial Sequence
  source: /note=“Description of Artificial Sequence: Synthetic polypeptide”
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 187 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 188 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 189 - protein, 120 aa, Artificial Sequence
  source: /note=“Description of Artificial Sequence: Synthetic polypeptide”
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 190 - protein, 107 aa, Artificial Sequence
  source: /note=“Description of Artificial Sequence: Synthetic polypeptide”
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 191 - protein, 119 aa, Artificial Sequence
  source: /note=“Description of Artificial Sequence: Synthetic polypeptide”
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 192 - protein, 110 aa, Artificial Sequence
  source: /note=“Description of Artificial Sequence: Synthetic polypeptide”
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 193 - protein, 121 aa, Artificial Sequence
  source: /note=“Description of Artificial Sequence: Synthetic polypeptide”
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 194 - protein, 107 aa, Artificial Sequence
  source: /note=“Description of Artificial Sequence: Synthetic polypeptide”
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFTFGQGTKLEIK

SEQ ID NO: 195 - protein, 119 aa, Artificial Sequence
  source: /note=“Description of Artificial Sequence: Synthetic polypeptide”
EVQLVQSGAEVKKPGSSVKVSCKASGFTFTDYYIHWVKQAPGQGLEWIGLVYPYIGGSSYNQQFKGKATLTVDTSTSTAYMELSSLRSEDTAVYYCARGDNNYDAMDYWGQGTTVTVSS

SEQ ID NO: 196 - protein, 110 aa, Artificial Sequence
  source: /note=“Description of Artificial Sequence: Synthetic polypeptide”
DIVLTQSPDSLAVSLGERATISCRASQSVSTSTYNYMHWYQQKPGQPPKLLVKYASNLESGVPDRFSGSGSGTDFTLTISSLQAEDVAVYYCHHTWEIRTFGGGTKVEIK

SEQ ID NO: 197 - protein, 120 aa, Artificial Sequence
  source: /note=“Description of Artificial Sequence: Synthetic polypeptide”
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 198 - protein, 112 aa, Artificial Sequence
  source: /note=“Description of Artificial Sequence: Synthetic polypeptide”
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 199 - protein, 5 aa, Artificial Sequence
  source: /note=“Description of Artificial Sequence: Synthetic peptide”
DYYIH

SEQ ID NO: 200 - protein, 17 aa, Artificial Sequence
  source: /note=“Description of Artificial Sequence: Synthetic peptide”
LVYPYIGGTNYNQKFKG

SEQ ID NO: 201 - protein, 10 aa, Artificial Sequence
  source: /note=“Description of Artificial Sequence: Synthetic peptide”
GDNKYDAMDY

SEQ ID NO: 202 - protein, 15 aa, Artificial Sequence
  source: /note=“Description of Artificial Sequence: Synthetic peptide”
RASQSVSTSSYSYMH

SEQ ID NO: 203 - protein, 7 aa, Artificial Sequence
  source: /note=“Description of Artificial Sequence: Synthetic peptide”
YASSLES

SEQ ID NO: 204 - protein, 8 aa, Artificial Sequence
  source: /note=“Description of Artificial Sequence: Synthetic peptide”
EQSWEIRT

SEQ ID NO: 205 - protein, 5 aa, Artificial Sequence
  source: /note=“Description of Artificial Sequence: Synthetic peptide”
NYWMH

SEQ ID NO: 206 - protein, 17 aa, Artificial Sequence
  source: /note=“Description of Artificial Sequence: Synthetic peptide”
MIHPNSGSTKHNEKFRG

SEQ ID NO: 207 - protein, 12 aa, Artificial Sequence
  source: /note=“Description of Artificial Sequence: Synthetic peptide”
SDFGNYRWYFDV

SEQ ID NO: 208 - protein, 11 aa, Artificial Sequence
  source: /note=“Description of Artificial Sequence: Synthetic peptide”
RASQSSSNNLH

SEQ ID NO: 209 - protein, 7 aa, Artificial Sequence
  source: /note=“Description of Artificial Sequence: Synthetic peptide”
YVSQSIS

SEQ ID NO: 210 - protein, 9 aa, Artificial Sequence
  source: /note=“Description of Artificial Sequence: Synthetic peptide”
QQSNSWPFT

SEQ ID NO: 211 - protein, 5 aa, Artificial Sequence
  source: /note=“Description of Artificial Sequence: Synthetic peptide”
DYYIH

SEQ ID NO: 212 - protein, 17 aa, Artificial Sequence
  source: /note=“Description of Artificial Sequence: Synthetic peptide”
LVYPYIGGSSYNQQFKG

SEQ ID NO: 213 - protein, 10 aa, Artificial Sequence
  source: /note=“Description of Artificial Sequence: Synthetic peptide”
GDNNYDAMDY

SEQ ID NO: 214 - protein, 15 aa, Artificial Sequence
  source: /note=“Description of Artificial Sequence: Synthetic peptide”
RASQSVSTSTYNYMH

SEQ ID NO: 215 - protein, 7 aa, Artificial Sequence
  source: /note=“Description of Artificial Sequence: Synthetic peptide”
YASNLES

SEQ ID NO: 216 - protein, 8 aa, Artificial Sequence
  source: /note=“Description of Artificial Sequence: Synthetic peptide”
HHTWEIRT

SEQ ID NO: 217 - protein, 5 aa, Artificial Sequence
  source: /note=“Description of Artificial Sequence: Synthetic peptide”
SYGVH

SEQ ID NO: 218 - protein, 16 aa, Artificial Sequence
  source: /note=“Description of Artificial Sequence: Synthetic peptide”
VIWAGGSTNYNSALMS

SEQ ID NO: 219 - protein, 12 aa, Artificial Sequence
  source: /note=“Description of Artificial Sequence: Synthetic peptide”
HMITEDYYGMDY

SEQ ID NO: 220 - protein, 17 aa, Artificial Sequence
  source: /note=“Description of Artificial Sequence: Synthetic peptide”
KSSQSLLNSRTRKNYLA

SEQ ID NO: 221 - protein, 7 aa, Artificial Sequence
  source: /note=“Description of Artificial Sequence: Synthetic peptide”
WASTRES

SEQ ID NO: 222 - protein, 8 aa, Artificial Sequence
  source: /note=“Description of Artificial Sequence: Synthetic peptide”
KQSYNLPT

SEQ ID NO: 223 - protein, 5 aa, Artificial Sequence
  source: /note=“Description of Artificial Sequence: Synthetic peptide”
NYWLG

SEQ ID NO: 224 - protein, 17 aa, Artificial Sequence
  source: /note=“Description of Artificial Sequence: Synthetic peptide”
DIYPGGGNTYYNEKLKG

SEQ ID NO: 225 - protein, 11 aa, Artificial Sequence
  source: /note=“Description of Artificial Sequence: Synthetic peptide”
WGDKKGNYFAY

SEQ ID NO: 226 - protein, 12 aa, Artificial Sequence
  source: /note=“Description of Artificial Sequence: Synthetic peptide”
TASSSVYSSYLH

SEQ ID NO: 227 - protein, 7 aa, Artificial Sequence
  source: /note=“Description of Artificial Sequence: Synthetic peptide”
STSNLAS

SEQ ID NO: 228 - protein, 8 aa, Artificial Sequence
  source: /note=“Description of Artificial Sequence: Synthetic peptide”
HQYHRSPT

SEQ ID NO: 229 - protein, 5 aa, Artificial Sequence
  source: /note=“Description of Artificial Sequence: Synthetic peptide”
NFGMN

SEQ ID NO: 230 - protein, 17 aa, Artificial Sequence
  source: /note=“Description of Artificial Sequence: Synthetic peptide”
WINLYTGEPTFADDFKG

SEQ ID NO: 231 - protein, 13 aa, Artificial Sequence
  source: /note=“Description of Artificial Sequence: Synthetic peptide”
KGETYYRYDGFAY

SEQ ID NO: 232 - protein, 16 aa, Artificial Sequence
  source: /note=“Description of Artificial Sequence: Synthetic peptide”
RSSKSLLHSNGNTHLY

SEQ ID NO: 233 - protein, 7 aa, Artificial Sequence
  source: /note=“Description of Artificial Sequence: Synthetic peptide”
RMSNLAS

SEQ ID NO: 234 - protein, 9 aa, Artificial Sequence
  source: /note=“Description of Artificial Sequence: Synthetic peptide”
MQLLEYPYT

SEQ ID NO: 235 - protein, 4 aa, Artificial Sequence
  source: /note=“Description of Artificial Sequence: Synthetic peptide”
GFLG

SEQ ID NO: 236 - protein, 4 aa, Artificial Sequence
  source: /note=“Description of Artificial Sequence: Synthetic peptide”
ALAL

(19) United States
(12) Patent Application Publication
(10) Pub. No.: US 2022/0362396 A1
(43) Pub. Date: Nov. 17, 2022

(54) AMINO-PYRAZINECARBOXAMIDE COMPOUNDS, CONJUGATES, AND USES THEREOF

(71) Applicant: Silverback Therapeutics, Inc., Seattle, WA (US)

(72) Inventors: Sean Wesley Smith, Seattle, WA (US); Craig Alan Coburn, Seattle, WA (US); Peter Robert Baum, Seattle, WA (US); Robert Finley Dubose, Seattle, WA (US)

(21) Appl. No.: 17/102,664

(22) Filed: Nov. 24, 2020

Related U.S. Application Data
(63) Continuation of application No. PCT/US19/34024, filed on May 24, 2019.
(60) Provisional application No. 62/676,832, filed on May 25, 2018.
(60) Provisional application No. 62/778,812, filed on Dec. 12, 2018.

(51) Int. Cl. A61K 47/68; A61K 31/5377; A61K 31/497; C07D 401/12; C07D 401/14; C07D 405/14; C07D 413/14; A61K 31/655; A61K 31/541; A61K 31/553; C07D 471/10; C07D 471/04; A61K 31/55; A61K 31/395; C07K 16/18; A61P 35/00; A61P 13/12

(52) U.S. Cl. CPC A61K 47/6851; A61K 2039/505; A61K 31/5377; A61K 31/497; C07D 401/12; C07D 401/14; C07D 405/14; C07D 413/14; A61K 31/655; A61K 31/541; A61K 31/553; C07D 471/10; C07D 471/04; A61K 31/55; A61K 31/5545; C07K 16/18; A61P 35/00; A61P 13/12; A61K 47/6803; A61K 47/6889; A61K 47/6849

(57) ABSTRACT

Amino-pyrazinecarboxamide compounds, conjugates, and pharmaceutical compositions for use in the treatment of disease, such as cancer, are disclosed herein. The disclosed compounds are useful, among other things, in the treating of cancer and fibrosis and modulating TGFβR2. Additionally, compounds incorporated into a conjugate with an antibody construct are described herein.

### CROSS-REFERENCE

This application is a Continuation of International Patent Application No. PCT/US19/34024, filed May 24, 2019, which claims the benefit of U.S. Provisional Application No. 62/676,832 filed on May 25, 2018, and U.S. Provisional Application No. 62/778,812 filed on Dec. 12, 2018, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 22, 2019, is named 50358-733_601_SL.txt and is 161,275 bytes in size.

### BACKGROUND OF THE INVENTION

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

Fibrosis is the formation of excess fibrous connective tissue or scar tissue in an organ or tissue in a reparative or reactive process. Fibrosis can occur in many tissues within the body, typically as a result of inflammation or damage, which include the lungs, liver, heart, and brain. Scar tissue blocks arteries, immobilizes joints and damages internal organs, wreaking havoc on the body's ability to maintain vital functions. Every year, millions of people are hospitalized due to the damaging effects of fibrosis. However, current therapeutics for treating fibrotic diseases are lacking or have drawbacks. Thus, there remains a considerable need for alternative or improved treatments for fibrotic diseases.

### SUMMARY OF THE INVENTION

The present disclosure generally relates to substituted amino-pyrazinecarboxamide compounds and pharmaceutical compositions. The substituted amino-pyrazinecarboxamide compounds may be used to treat or prevent cancer and/or fibrotic diseases. The disclosed amino-pyrazinecarboxamide compounds may inhibit TGFβ1, TGFβR1, TGFβR2, or combinations thereof. The disclosed amino-pyrazinecarboxamide compounds may be incorporated into conjugates, such as antibody conjugates.

In one aspect of the invention is a compound represented by Formula (I):

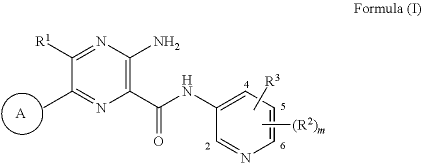

or a pharmaceutically acceptable salt thereof, wherein:
Ring A is unsubstituted or substituted cycloalkyl, unsubstituted or substituted heterocycloalkyl, unsubstituted or substituted aryl, or unsubstituted or substituted heteroaryl, wherein when Ring A is substituted, substituents on Ring A are independently selected at each occurrence from R⁴;
   each R⁴ is selected from R^{L} and R²⁰, or two R⁴ on adjacent atoms are taken together with the atoms to which they are attached to form an unsubstituted or substituted monocyclic carbocycle or unsubstituted or substituted monocyclic heterocycle;
   R^{L} is

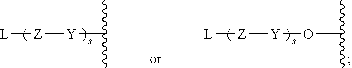

each Y is independently unsubstituted or substituted C₁-C₆alkylene; wherein when Y is substituted, substituents on Y are independently selected at each occurrence from R⁵; each R⁵ is selected from R²⁰, or two R⁵ on adjacent atoms are taken together with the atoms to which they are attached to form an unsubstituted or substituted monocyclic carbocycle, or unsubstituted or substituted monocyclic heterocycle;
each Z is independently —NR⁶S(═O)₂—, —S(═O)₂NR⁶—, —OC(═O)—, —C(═O)O—, —C(═O)NR⁶—, or —NR⁶C(═O)—; wherein each R⁶ is independently selected from hydrogen, unsubstituted or substituted C₁-C₆alkyl, unsubstituted or substituted carbocycle, and unsubstituted or substituted heterocycle, or an R⁵ and an R⁶ on adjacent atoms are taken together with the atoms to which they are attached to form an unsubstituted or substituted monocyclic heterocycle;
L is unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; wherein when L is substituted, substituents on L are independently selected at each occurrence from R⁷;
each R⁷ is selected from —SSR⁵⁰ and R²⁰;
s is 1-10;
R¹ is selected from hydrogen and R²⁰;
each R² is independently selected from R²⁰, or two R² on adjacent atoms are taken together with the atoms to which they are attached to form an unsubstituted or substituted monocyclic carbocycle or unsubstituted or substituted monocyclic heterocycle;
m is 0-3;
R³ is selected from (i), (ii), (iii), and (iv):
   (i) unsubstituted or substituted aryl, or unsubstituted or substituted heteroaryl; wherein when R³ is substituted, substituents on R³ are independently selected at each occurrence from R¹⁰;
   (ii) unsubstituted or substituted cycloalkyl, or unsubstituted or substituted heterocycloalkyl; wherein when R³ is substituted, substituents on R³ are independently selected at each occurrence from R¹¹;
   (iii) unsubstituted or substituted polycyclic heterocycloalkyl, unsubstituted or substituted 3- to 5-membered monocyclic heterocycloalkyl, unsubstituted or substituted 6- to 8-membered monocyclic heterocycloalkyl comprising 1 or 2 N atoms and 1 or 2 other heteroatoms selected from O or S; wherein when R³ is substituted, substituents on R³ are independently selected at each occurrence from R¹; and

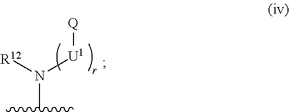

wherein when R³ is at the 2-, 5-, or 6-position of the pyridine, R³ is selected from (i), (ii), and (iv), and when R³ is at the 4-position of the pyridine, R³ is selected from (i), (iii), and (iv); and
   each R¹⁰ is selected from R²⁰or two R¹⁰ on adjacent atoms are taken together with the atoms to which they are attached to form an unsubstituted or substituted monocyclic carbocycle or unsubstituted or substituted monocyclic heterocycle;
   each R¹¹ is selected from ═O, ═S, and R²⁰;
   R¹² is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   Q is —OR¹³, —NR¹³R¹³, —SR¹³, —CN, —C(═O)R¹⁴, —C(═O)NR¹³R¹³, —S(═O)R¹⁴, or —S(═O)₂R¹⁴, or —S(═O)₂NR¹³R¹³.
      R¹³ is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
      R¹⁴ is unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   each U¹ is —(CR¹⁵R¹⁶)—, wherein each R¹⁵ and R¹⁶ are independently selected from hydrogen and R²⁰;
   r is 1-5;
each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
each R⁵⁰ is independently selected from unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
each R⁵¹ is independently selected from hydrogen, unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
or two R⁵¹ on the same N atom are taken together with the N atom to which they are attached to form an unsubstituted or substituted N-containing heterocycle;
   wherein when any of R², R⁴, R⁵, R⁶, R¹⁰, R¹², R¹³, R¹⁴, R²⁰, R⁵⁰, and R⁵¹ are substituted, substituents on the R², R⁴, R⁵, R⁶, R¹⁰, R¹², R¹³, R¹⁴, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted or substituted C₁-C₆alkyl, C₁-C₆haloalkyl, unsubstituted or substituted monocyclic carbocycle, unsubstituted or substituted monocyclic heterocycle, or two substituents on the same carbon atom are taken together to form a C═O or C═S and wherein substituents on said C₁-C₆alkyl are independently selected from R⁵⁴ and substituents on said carbocycle and heterocycle are independently selected from R⁵⁵;
   each R⁵² is independently selected from hydrogen, C₁-C₆alkyl, C₃-C₆cycloalkyl, phenyl, benzyl, 5-membered heteroaryl, and 6-membered heteroaryl;
   or two R⁵² groups are taken together with the N atom to which they are attached to form a N-containing heterocycle; and
   each R⁵³ is independently selected from C₁-C₆alkyl, C₃-C₆cycloalkyl, phenyl, benzyl, 5-membered heteroaryl, and 6-membered heteroaryl.
   each R⁵⁴ is independently selected from —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², and phenyl;
   each R⁵⁵ is independently selected from —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², and unsubstituted or substituted C₁-C₆alkyl wherein substituents on said C₁-C₆alkyl are independently selected from R⁵⁴.

Also included are Compounds of Formula (I) or a pharmaceutically acceptable salt thereof, wherein:
Ring A is unsubstituted or substituted cycloalkyl, unsubstituted or substituted heterocycloalkyl, unsubstituted or substituted aryl, or unsubstituted or substituted heteroaryl, wherein when Ring A is substituted, substituents on Ring A are independently selected at each occurrence from R⁴;
   each R⁴ is selected from R^{L} and R²⁰, or two R⁴ on adjacent atoms are taken together with the atoms to which they are attached to form an unsubstituted or substituted monocyclic carbocycle or unsubstituted or substituted monocyclic heterocycle;
   R^{L} is

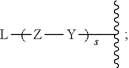

each Y is independently unsubstituted or substituted C₁-C₆alkylene; wherein when Y is substituted, substituents on Y are independently selected at each occurrence from R⁵;
   each R⁵ is selected from R²⁰, or two R⁵ on adjacent atoms are taken together with the atoms to which they are attached to form an unsubstituted or substituted monocyclic carbocycle, or unsubstituted or substituted monocyclic heterocycle;
each Z is independently —NR⁶S(═O)₂—, —S(═O)₂NR⁶—, —OC(═O)—, —C(═O)O—, —C(═O)NR⁶—, or —NR⁶C(═O)—; wherein each R⁶ is independently selected from hydrogen, unsubstituted or substituted C₁-C₆alkyl, unsubstituted or substituted carbocycle, and unsubstituted or substituted heterocycle, or an R⁵ and an R⁶ on adjacent atoms are taken together with the atoms to which they are attached to form an unsubstituted or substituted monocyclic heterocycle;
L is unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; wherein when L is substituted, substituents on L are independently selected at each occurrence from R⁷;
   each R⁷ is selected from —SSR⁵⁰ and R²⁰;
s is 1-10;
R¹ is selected from hydrogen and R²⁰;
each R² is independently selected from R²⁰, or two R² on adjacent atoms are taken together with the atoms to which they are attached to form an unsubstituted or substituted monocyclic carbocycle or unsubstituted or substituted monocyclic heterocycle;
m is 0-3;
R³ is selected from (i), (ii), (iii), and (iv):
   (i) unsubstituted or substituted aryl, or unsubstituted or substituted heteroaryl; wherein when R³ is substituted, substituents on R³ are independently selected at each occurrence from R¹⁰;
   (ii) unsubstituted or substituted cycloalkyl, or unsubstituted or substituted heterocycloalkyl; wherein when R³ is substituted, substituents on R³ are independently selected at each occurrence from R¹¹;
   (iii) unsubstituted or substituted polycyclic heterocycloalkyl, unsubstituted or substituted 3- to 5-membered monocyclic heterocycloalkyl, unsubstituted or substituted 6- to 8-membered monocyclic heterocycloalkyl comprising 1 or 2 N atoms and 1 or 2 other heteroatoms selected from O or S; wherein when R³ is substituted, substituents on R³ are independently selected at each occurrence from R¹¹; and

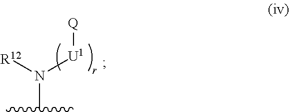

wherein when R³ is at the 2-, 5-, or 6-position of the pyridine, R³ is selected from (i), (ii), and (iv), and when R³ is at the 4-position of the pyridine, R³ is selected from (i), (iii), and (iv); and
   each R¹⁰ is selected from R²⁰, or two R¹⁰ on adjacent atoms are taken together with the atoms to which they are attached to form an unsubstituted or substituted monocyclic carbocycle or unsubstituted or substituted monocyclic heterocycle;
   each R¹ is selected from ═O, ═S, and R²⁰;
   R¹² is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   Q is —OR¹³, —NR¹³R¹³, —SR¹³, —CN, —C(═O)R¹⁴, —C(═O)NR¹³R¹³, —S(═O)R¹⁴, or —S(═O)₂R¹⁴, or —S(═O)₂NR¹³R¹³.
      R¹³ is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
      R¹⁴ is unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   each U¹ is —(CR¹⁵R¹⁶)—, wherein each R¹⁵ and R¹⁶ are independently selected from hydrogen and R²⁰;
   r is 1-5;
each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
each R⁵⁰ is independently selected from unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
each R⁵¹ is independently selected from hydrogen, unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
or two R⁵¹ on the same N atom are taken together with the N atom to which they are attached to form an unsubstituted or substituted N-containing heterocycle;
wherein when any of R², R⁴, R⁵, R⁶, R¹⁰, R¹², R¹³, R¹⁴, R²⁰, R⁵⁰, and R⁵¹ are substituted, substituents on the R², R⁴, R⁵, R⁶, R¹⁰, R¹², R¹³, R¹⁴, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², C₁-C₆alkyl, C₁-C₆haloalkyl, monocyclic carbocycle, and monocyclic heterocycle; or two substituents on the same carbon atom are taken together to form a C═O or C═S;
   each R⁵² is independently selected from hydrogen, C₁-C₆alkyl, C₃-C₆cycloalkyl, phenyl, benzyl, 5-membered heteroaryl, and 6-membered heteroaryl;
   or two R⁵² groups are taken together with the N atom to which they are attached to form a N-containing heterocycle; and
   each R⁵³ is independently selected from C₁-C₆alkyl, C₃-C₆cycloalkyl, phenyl, benzyl, 5-membered heteroaryl, and 6-membered heteroaryl.

In certain embodiments, the compound of Formula (I) is represented by Formula (II):

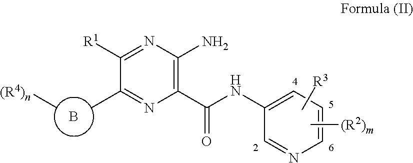

wherein:
ring B is aryl or heteroaryl; and
n is 0-5.

In some embodiments, compounds disclosed herein are attached to a linker to form compound-linkers.

In some embodiments, compounds disclosed herein are covalently bound to an antibody construct or a targeting moiety, optionally via a linker.

Also disclosed herein are pharmaceutical compositions of the compounds or conjugates described herein.

In some aspects, the present disclosure provides a method for treating cancer, comprising administering a compound, a conjugate, or a pharmaceutical composition as described herein to a subject in need thereof.

In some aspects, the present disclosure provides a method for enhancing an immune response (e.g., an anti-cancer immune response) in a subject comprising administering a compound, a conjugate, or a pharmaceutical composition as described herein to a subject in need thereof.

In some aspects, the present disclosure provides a method for treating fibrosis, comprising administering a compound, a conjugate, or a pharmaceutical composition as described herein to a subject in need thereof. In some aspects, the fibrosis is cancer-associated. In some aspects, the fibrosis is not cancer-associated. In one aspect, the fibrosis is scleroderma. In another aspect, the fibrosis is systemic fibrosis. In one aspect, the fibrotic disease is steatohepatitis, e.g., non-alcoholic steatohepatitis (NASH).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1 illustrates that an exemplary TGFβR2 inhibitor conjugated to an anti-LRRC15 antibody through either cleavable or non-cleavable linkers inhibits TGFβ-induced SMAD2 promoter activity in a reporter assay. A TGFβ/SMAD promoter-luciferase reporter line stably transfected with human LRRC15 was treated with conjugates and control antibodies at indicated concentrations for 24 hours followed by TGFβ for 18 hours. Luciferase activity in treated samples was determined by a chemiluminescence assay and extent of inhibition determined by the relative reduction of chemiluminescence compared to samples receiving only buffer then TGFβ.

FIG. 2 shows that in a concentration dependent manner selected compounds inhibit TGFβ-induction of αSMA gene expression in a human lung fibroblast cell line derived from an IPF patient. LL97a cells were treated with TGFβ and selected compounds or DMSO carrier at indicated concentrations for 24 hours before implementation of qPCR to determine αSMA mRNA levels. Upper and lower dashed lines indicate 100% and 50% level of αSMA mRNA induction in cells treated with TGFβ only.

FIG. 3 shows that in a concentration dependent manner compound 250 inhibits TGFβ-induction of αSMA gene expression in a human lung fibroblast cell line derived from an IPF patient. LL97a cells were treated with TGFβ and Compound 250 or DMSO carrier at indicated concentrations for 24 hours before implementation of qPCR to determine αSMA mRNA levels. Upper and lower dashed lines indicate 100% and 50% level of αSMA mRNA induction in cells treated with TGFβ only.

FIG. 4 shows that in a concentration dependent manner compound 250 inhibits TGFβ-induction of elastin gene expression in a human lung fibroblast cell line derived from an IPF patient. LL97a cells were treated with TGFβ and Compound 250 or DMSO carrier at indicated concentrations for 24 hours before implementation of qPCR to determine elastin mRNA levels. Upper and lower dashed lines indicate 100% and 50% level of elastin mRNA induction in cells treated with TGFβ only.

FIG. 5 shows that in a concentration dependent manner selected compounds inhibit TGFβ-induction of αSMA gene expression in a human lung fibroblast cell line derived from an IPF patient. LL97a cells were treated with TGFβ and selected compounds or DMSO carrier at indicated concentrations for 24 hours before implementation of qPCR to determine αSMA mRNA levels. Upper and lower dashed lines indicate 100% and 50% level of αSMA mRNA induction in cells treated with TGFβ only.

FIGS. 6A-B show that antibody conjugates of selected compounds linked to cysteines with PABC cleavable linkers at a high average DAR have high potency inhibiting TGFβ-induction of αSMA gene expression in LL97a cells (A) or elastin gene expression in NHFL cells (B). Cells were treated with TGFβ and conjugates or controls at indicated concentrations for 24 hours before RNA was prepared and subjected to qPCR. Upper and lower dashed lines indicate 100% and 50% level of mRNA induction in cells treated with only buffer and TGFβ.

FIG. 7A-B show that similar average DAR conjugates of LP1 to an anti-LRRC15 antibody with a wild type Fc (asterisk) or to the antibody with a null Fc domain have similar potency in reducing TGFβ-induced αSMA gene expression in LL97a cells (A) or elastin gene expression in normal human lung fibroblast (NHLF) cells (B). Cells were treated with conjugates and controls at indicated concentrations plus TGFβ for 24 hours (A) or 48 hours (B) before RNA was prepared and subjected to qPCR. Upper and lower dashed lines indicate 100% and 50% level of mRNA induction in cells treated only with buffer and TGFβ

FIGS. 8A-C show that intratumoral injections of compound 211 reduces the mRNA level of select TGFβ-inducible genes in mice inoculated with PANC-1 tumor cells (A), of compounds 171 and 211 reduce the mRNA level of select TGFβ-inducible genes in mice inoculated with BxPC3 tumor cells (B) and of compound 211 reduces the mRNA level of select TGFβ-inducible genes in mice inoculated with BxPC3 tumor cells (C). Asterisks denote a statistically significant reduction of gene mRNA was found after treatment with the compound compared to DMSO carrier control treated animals.

FIG. 9 shows that systemic dosing of anti-LRRC15 conjugates LP35 and LP36 reduce the mRNA level of select TGFβ-regulated genes within tumors of mice inoculated with BxPC3 tumor cells. Animals were dosed intravenously with either conjugate or controls of the unconjugated antibody or an irrelevant antibody isotype control. Asterisks denote a statistically significant reduction of select gene mRNA within the tumors was found after treatment with the conjugate compared to control animals receiving doses of the unconjugated antibody.

FIG. 10 shows that systemic dosing anti-LRRC15 conjugate LP36 reduces the mRNA level of select TGFβ-regulated genes within tumors of mice inoculated with BxPC3 tumor cells at doses of 5 mpk and 20 milligrams per kilogram. Animals were dosed intravenously with either conjugate or controls of the unconjugated antibody or an irrelevant antibody isotype control at indicated dose levels. Asterisks denote a statistically significant reduction of select gene mRNA within the tumors was found after treatment with the conjugate compared to control animals receiving 20 mpk doses of the unconjugated antibody.

FIG. 11 shows that systemic administration of the anti-LRRC15 conjugate LP1 decreases histopathological fibrosis in a model of systemic scleroderma. Mice received daily intradermal injections of bleomycin for 22 days. Treatment of animals with either ip injections of 10 mpk of conjugate or in PBS was initiated on d14 after the onset on fibrosis. After sacrifice of animals on d23 fixed dermal tissue was scored for fibrosis after Masson's Trichrome Stain by histopathology. As shown treatment with the conjugate significantly lowered fibrosis by this measure compared to the PBS control animals.

FIG. 12 shows that systemic administration of the anti-LRRC15 conjugate LP1 decreases fibrosis in a model of systemic fibrosis Mice received daily intradermal injections of bleomycin for 22 days. Treatment of animals with either ip injections of 10 mpk of conjugate or in PBS was initiated on d14 after the onset on fibrosis. After sacrifice of animals on d23 dermal tissue was subjected to a Sircol Red collagen content assay. As shown treatment with the conjugate lowered fibrosis by this measure compared to the PBS control animals.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Transforming growth factors (TGFs) and their receptors (TGFRs) are evolutionarily conserved molecules that play important, pleiotropic roles in the regulation of numerous development and physiological pathways, such as cell proliferation, cell differentiation, embryonic development, extracellular matrix formation, wound healing, bone development, immune responses, and inflammatory responses. Given the breadth of their biological functions, TGFs and TGFRs are also involved in many pathological processes, such as those underlying the development and progression of cancer, immune and inflammatory diseases, fibrosis, scarring, atherosclerosis, viral infections, and others.

Transforming growth factor beta-1 (TGFβ1) is the prototypical member of the TGF superfamily of ligands. TGFβ1 is a growth factor and cytokine involved in signaling within a broad array of tissue types. Overexpression of TGFβ1 has been shown to induce fibrotic disease pathology in a number of organ systems, including the kidney, liver, heart, lung, bone marrow, and skin.

TGFβ1 plays numerous roles in tumor progression. TGFβ1 can induce epithelial to mesenchymal transition, enhance the ability of tumor cells to grow, influence tumor cell fate, and modulate the composition of the tumor microenvironment so that it is more permissive to tumor growth.

TGFβ1 plays a role in the maintenance of peripheral tolerance in T-cells and in the prevention of maturation of dendritic cells. Further, TGFβ1 has been shown to regulate the antigen-presentation functions of dendritic cells by down-regulating expression of Major Histocompatibility Complex class II (MHC-II) and the secretion of Interleukin-12 (IL-12).

TGFβ1 signaling by its receptors in myeloid cells has been shown to play roles in tumor promotion and tumor immune suppression including in dendritic cells, myeloid-derived suppressor cells, tumor associated macrophages or combinations of these cells.

Transforming growth factor beta receptor 2 (TGFβR2) is one of two transmembrane serine/threonine kinase receptors that are required for TGFβ1 signal transduction, with the other receptor being TGFβR1. TGFβ1 first binds to TGFβR2 at the plasma membrane, inducing the formation of the TGFβR1-TGFβR2 complex, which leads to phosphorylation of Mothers Against Decapentaplegic homolog 2 (Smad2) and Mothers Against Decapentaplegic homolog 3 (Smad3), and subsequent modulation of a number of downstream signaling targets.

Given the wide range of pathological cellular and multicellular interactions in which TGFβ1 plays a prominent role, pharmacological inhibition of TGFβ1 or its receptors, TGFβR1 or TGFβR2, may prove to be useful in the treatment of several diseases.

Challenges to developing targeted therapies include achieving high selectivity for the primary pharmacological target and maintaining prolonged target inhibition. In overcoming these two challenges, it is possible to develop pharmaceutical products with increased therapeutic efficacy and reduced systemic toxicity. One approach to addressing these two challenges is developing covalent drugs, whereby a covalent interaction takes place between the pharmacological entity and a specific cysteine in the active site of the protein target.

There is a current need for therapeutics that can inhibit TGFβ1, TGFβR1, TGFβR2, or combinations thereof, treat or prevent cancer, and treat or prevent fibrosis. The fibrosis may or may not be associated with cancer. The present disclosure provides compounds, compositions and methods that address this need and related needs.

The present disclosure provides compounds, conjugates, and pharmaceutical compositions for use in the treatment or prevention of disease. In certain embodiments, the substituted amino-pyrazinecarboxamide compounds, conjugates, and pharmaceutical compositions are used in the treatment or prevention of disease, such as cancer and fibrotic diseases. The substituted amino-pyrazinecarboxamide compounds and conjugates thereof may be useful, among other things, in treating and preventing cancer, treating and preventing fibrotic diseases, and modulating TGFβ1, TGFβR1, TGFβR2, or combinations thereof. The substituted amino-pyrazinecarboxamide compounds may useful in inhibiting TGFβ1, TGFβR1, TGFβR2, or combinations thereof. The amino-pyrazinecarboxamide compounds may be incorporated into conjugates, such as antibody conjugates.

The compounds of the present disclosure, as well as conjugates thereof, may be useful for the treatment and prevention, e.g., vaccination, of cancer, autoimmune diseases, inflammation, sepsis, allergy, asthma, graft rejection, graft-versus-host disease, fibrosis, immunodeficiencies, and infectious diseases.

In certain embodiments, the compounds have utility in the treatment of cancer either as single agents, as conjugates, or in combination therapy. In certain embodiments, the compounds have utility as single agent immunomodulators, vaccine adjuvants and in combination with conventional cancer therapies. In certain embodiments, the compounds are attached to an antibody construct to form a conjugate that can be utilized, for example, to enhance an immune response or for treating fibrosis. In certain embodiments, the disclosure provides antibody construct-amino-pyrazinecarboxamide compound conjugates (conjugates), and their use for treating cancer or fibrosis.

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. The antigen binding fragment can include, for example, a Fab′, F(ab′)₂, Fab, Fv, rIgG, and scFv.

As used herein, an “antigen binding domain” refers to a region of a molecule that specifically binds to an antigen. An antigen binding domain can be an antigen-binding portion of an antibody or an antibody fragment. An antigen binding domain can be one or more fragments of an antibody that can retain the ability to specifically bind to an antigen. An antigen binding domain can be an antigen binding fragment. In some embodiments, an antigen binding domain can recognize a single antigen. An antigen binding domain can recognize, for example, two or three antigens.

As used herein, an “antibody construct” refers to a molecule, e.g., a protein, peptide, antibody or portion thereof, that contains an antigen binding domain and an Fc domain.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val).

As used herein, “conjugate” refers to an antibody construct that is attached (e.g., conjugated) either directly or through a linker group to a compound described herein, e.g., a compound or salt of any one of Formulas (I-A), (I-B), (I-C), (I-D), (I-E), (II-A), (II-B), (II-C), and (II-D) and Table 14.

As used herein, an “Fc domain” can be an Fc domain from an antibody or from a non-antibody that can bind to an Fc receptor.

As used herein, “recognize” with regard to antibody interactions refers to the association or binding between an antigen binding domain of an antibody or portion thereof and an antigen.

As used herein, “sequence identity” refers to the identity between a DNA, RNA, nucleotide, amino acid, or protein sequence to another DNA, RNA, nucleotide, amino acid, or protein sequence, respectively, according to context. Sequence identity can be expressed in terms of a percentage of sequence identity of a first sequence to a second sequence. Percent (%) sequence identity with respect to a reference DNA sequence is the percentage of DNA nucleotides in a candidate sequence that are identical with the DNA nucleotides in the reference DNA sequence after aligning the sequences and introducing gaps, as necessary. Percent (%) sequence identity with respect to a reference amino acid sequence is the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, “specifically binds” and the like refers to the specific association or specific binding between the antigen binding domain and the antigen, as compared with the interaction of the antigen binding domain with a different antigen (i.e., non-specific binding). In some embodiments, an antigen binding domain that recognizes or specifically binds to an antigen has a dissociation constant (KD) of <<100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M).

As used herein, a “target binding domain” refers to a construct that contains an antigen binding domain from an antibody or from a non-antibody that can bind to the antigen.

The term “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target molecule may be an antigen, such as a biological receptor or other structure of a cell such as a tumor antigen.

As used herein, a “tumor antigen” can be an antigenic substance associated with a tumor or cancer cell, and can trigger an immune response in a host.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons, respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a straight divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar” (such as in “aralkyl”) is meant to include aryl radicals optionally substituted by one or more substituents such as those substituents described herein.

The term “cycloalkyl” refers to a saturated ring in which each atom of the ring is carbon. Cycloalkyl may include monocyclic and polycyclic rings such as 3- to 10-membered monocyclic rings, 6- to 12-membered bicyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), decalinyl, 7,7 dimethyl bicyclo[2.2.1]heptanyl, and the like. Unless otherwise stated specifically in the specification, the term “cycloalkyl” is meant to include cycloalkyl radicals that are optionally substituted by one or more substituents such as those substituents described herein.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 6- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl. Unless otherwise stated specifically in the specification, the term “cycloalkenyl” is meant to include cycloalkenyl radicals that are optionally substituted by one or more substituents such as those substituents described herein.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents such as those substituents described herein.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 6- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine. Unless otherwise stated specifically in the specification, the term “heterocycloalkenyl” is meant to include heterocycloalkenyl radicals that are optionally substituted by one or more substituents such as those substituents described herein.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3.sup.rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative amino or amine protecting groups include, formyl, acyl groups (such as acetyl, trifluoroacetyl, and benzoyl), benzyl, alkoxycarbonyl (such as benzyloxycarbonyl (CBZ), and tert-butoxycarbonyl (Boc)), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), sulfonyl, and the like. Compounds described herein can include protecting groups (e.g., a hydrogen on a reactive nitrogen atom of a compound described herein can be replaced by an amino protecting group).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

### Definitions of Genes and Proteins

CTLA4 gene encodes CTLA4 protein (cytotoxic T-lymphocyte-associated protein 4), also known as CD152 (cluster of differentiation 152), which is a protein receptor that acts as an immune checkpoint and downregulates immune responses. CTLA4 is constitutively expressed in Tregs but only upregulated in conventional T cells after activation. CTLA4 acts as an “off” switch when bound to CD80 or CD86 on the surface of antigen-presenting cells. The monoclonal antibody Ipilimamab has been developed to target CTLA4.

PDCD1 encodes programmed cell death protein 1, also known as PD-1 and CD279 (cluster of differentiation 279), which is a cell surface receptor that plays a cell surface receptor that plays an important role in down-regulating the immune system and promoting self-tolerance by suppressing T cell inflammatory activity. PD-1 is a cell surface receptor that belongs to the immunoglobulin superfamily and is expressed on T cells and pro-B cells. PD-1 is an immune checkpoint and guards against autoimmunity through a dual mechanism of promoting apoptosis (programmed cell death) in antigen specific T-cells in lymph nodes while simultaneously reducing apoptosis in regulatory T cells (anti-inflammatory, suppressive T cells). The human IgG4 anti-PD-1 monoclonal antibody Opdivo® (nivolumab) and humanized antibody Keytruda® (pembrolizumab) have been developed to target PD-1. The antibodies pidilizumab (CT-011, Cure Tech) and BMS-936559 are in clinical development.

CD274 encodes PD-L1 (programmed death-ligand 1), also known as CD274 (cluster of differentiation 274). PD-L1 is a 40 kDa type 1 transmembrane protein that has been speculated to play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2. The monoclonal antibodies Atezolizumab, Durvalumab, avelumab, and MDX-1106 have been developed to target PD-L1.

TNFR2 (tumor necrosis factor receptor 2), also known as TNFRSFlB (tumor necrosis factor receptor super family 1B) and CD120b, is a single-pass type I membrane protein and the member of TNFR superfamily containing 4 cysteine-rich domains (CRD) repeats. In addition to the full length membrane-anchored form, soluble TNFR2 can be generated via two distinct mechanisms: (1) shedding via proteolytic processing of the full membrane anchored from, and (2) translation from an alternatively spliced message encoding the extracellular domains of TNFR2. TNFR2 is the receptor with high affinity for TNF-alpha and approximately 5-fold lower affinity for homotrimeric lymphotoxin-alpha. The mouse monoclonal antibodies against TNFR2 described by SEQ ID NO: 56-SEQ ID NO: 82, and SEQ ID NO: 95-SEQ ID NO: 103, and anti-TNFR2 antibodies described by SEQ ID NO: 104 and SEQ ID NO: 105 have been developed to target TNFR2.

TNFRSF4 encodes OX40, also known as TNFRSF4 (tumor necrosis factor receptor superfamily, member 4), a member of the TNFR-superfamily of receptors which is not constitutively expressed on resting naïve T cells, unlike CD28. OX40 is a secondary costimulatory immune checkpoint molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. The monoclonal antibody Vonlerolizumab has been developed to target OX40.

CD27 is a member of the tumor necrosis factor receptor superfamily. The protein encoded by this gene is a member of the TNF-receptor superfamily. This receptor is required for generation and long-term maintenance of T cell immunity. It binds to ligand CD70, and plays a key role in regulating B-cell activation and immunoglobulin synthesis. This receptor transduces signals that lead to the activation of NF-κB and MAPK8/JNK. Adaptor proteins TRAF2 and TRAF5 have been shown to mediate the signaling process of this receptor. CD27-binding protein (SIVA), a proapoptotic protein, can bind to this receptor and is thought to play an important role in the apoptosis induced by this receptor. The monoclonal antibody Varlilumab has been developed to target CD27.

IL2RA encodes CD25, also known as IL2RA (interleukin-2 receptor alpha chain), which is a type I transmembrane protein present on activated T cells, activated B cells, some thymocytes, myeloid precursors, and oligodendrocytes. IL2RA is expressed in most B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Its soluble form, called sIL-2R may be elevated in these diseases and is occasionally used to track disease progression. The humanized monoclonal antibody Zinbryta® (Daclizumab) has been developed to target CD25.

TNFRSF18 encodes GITR (glucocorticoid-induced TNFR-related protein), also known as TNFRSF18 (tumor necrosis factor receptor superfamily member 18) and AITR (activation-inducible TNFR family receptor), which is a protein that is a member of the tumor necrosis factor receptor (TNF-R) superfamily. GITR (glucocorticoid-induced tumor necrosis factor receptor) is a surface receptor molecule that has been shown to be involved in inhibiting the suppressive activity of T-regulatory cells and extending the survival of T-effector cells. The anti-GITR antibodies described by SEQ ID NO: 37-SEQ ID NO: 42 and SEQ ID NO: 187-SEQ ID NO: 188, and antibody TRX518 have been developed to target GITR.

LAG-3 (lymphocyte-activation gene 3) encodes a cell surface molecule with diverse biologic effects on T cell function. LAG-3 is an immune checkpoint receptor. The LAG3 protein, which belongs to immunoglobulin (Ig) superfamily, comprises a 503-amino acid type I transmembrane protein with four extracellular Ig-like domains, designated D1 to D4. LAG-3 is expressed on activated T cells, natural killer cells, B cells and plasmacytoid dendritic cells. The anti-LAG-3 antibodies described by SEQ ID NO: 43-SEQ ID NO: 48 and SEQ ID NO: 111-SEQ ID NO: 112 have been developed to target LAG-3.

GARP (glycoprotein A repetitions predominant) is a transmembrane protein containing leucine rich repeats, which is present on the surface of stimulated Treg clones but not on Th clones. The anti-GARP antibodies described by SEQ ID NO: 113-SEQ ID NO: 122 have been developed to target GARP.

4-1BB is a type 2 transmembrane glycoprotein belonging to the TNF superfamily, expressed on activated T Lymphocytes. 4-1BB can be expressed by activated T cells. 4-1BB expression can be found on dendritic cells, B cells, follicular dendritic cells, natural killer cells, granulocytes and cells of blood vessel walls at sites of inflammation. The anti-4-1BB antibodies described by SEQ ID NO: 50-SEQ ID NO: 55 and SEQ ID NO: 123-SEQ ID NO: 128 have been developed to target 4-1BB.

ICOS (Inducible T-cell COStimulator) encodes a CD28-superfamily costimulatory molecule that is expressed on activated T cells. The protein encoded by this gene belongs to the CD28 and CTLA-4 cell-surface receptor family. ICOS forms homodimers and plays an important role in cell-cell signaling, immune responses and regulation of cell proliferation. The anti-ICOS antibodies described by SEQ ID NO: 129-SEQ ID NO: 132 have been developed to target ICOS.

CD70 is expressed on highly activated lymphocytes, such as in T- and B-cell lymphomas. CD70 is a cytokine that belongs to the tumor necrosis factor (TNF) ligand family. This cytokine is a ligand for TNFRSF27/CD27. It is a surface antigen on activated, but not on resting, T and B lymphocytes. CD70 induces proliferation of co-stimulated T cells, enhances the generation of cytolytic T cells, and contributes to T cell activation. This cytokine is also reported to play a role in regulating B-cell activation, cytotoxic function of natural killer cells, and immunoglobulin synthesis. The monoclonal antibody Vorsetuzumab has been developed to target CD70.

PDGFRβ (beta-type platelet-derived growth factor receptor) encodes a typical receptor tyrosine kinase, which is a transmembrane protein consisting of an extracellular ligand binding domain, a transmembrane domain and an intracellular tyrosine kinase domain. The molecular mass of the mature, glycosylated PDGFRβ protein is approximately 180 kDA. The monoclonal antibody Rinucumab has been developed to target PDGFRβ.

CD73 (cluster of differentiation 73), known as ecto-5′-nucleotidase (ecto-5′-NT, EC 3.1.3.5) is a glycosyl-phosphatidylinositol (GPI)-linked 70-kDa cell surface enzyme found in most tissues. CD73 commonly serves to convert AMP to adenosine. Ecto-5-prime-nucleotidase (5-prime-ribonucleotide phosphohydrolase; EC 3.1.3.5) catalyzes the conversion at neutral pH of purine 5-prime mononucleotides to nucleosides, the preferred substrate being AMP. The enzyme consists of a dimer of 2 identical 70-kD subunits bound by a glycosyl phosphatidyl inositol linkage to the external face of the plasma membrane. The enzyme is used as a marker of lymphocyte differentiation. The monoclonal antibody Oleclumab and the anti-CD73 antibodies described in SEQ ID NO: 139-SEQ ID NO: 140 have been developed to target CD73.

CD38 (cluster of differentiation 38), also known as cyclic ADP ribose hydrolase, is a glycoprotein found on the surface of many immune cells (white blood cells), including CD4⁺, CD8⁺, B lymphocytes and natural killer cells. CD38 also functions in cell adhesion, signal transduction and calcium signaling. The loss of CD38 function is associated with impaired immune responses, metabolic disturbances, and behavioral modifications including social amnesia possibly related to autism. The CD38 protein is a marker of cell activation. It has been connected to HIV infection, leukemias, myelomas, solid tumors, type II diabetes mellitus and bone metabolism, as well as some genetically determined conditions. CD38 produces an enzyme which regulates the release of oxytocin within the central nervous system. The monoclonal antibody Daratumumab has been developed to target CD38.

Integrin αvβ3 is a type of integrin that is a receptor for vitronectin. Integrin αvβ3 consists of two components, integrin alpha V and integrin beta 3 (CD61), and is expressed by platelets. Integrin αvβ3 is a receptor for phagocytosis on macrophages or dendritic cells. The monoclonal antibodies Etaracizumab and Intetumumab have been developed to target Integrin αvβ3.

Integrin αvβ8, a VN receptor, is identified as a potential negative regulator of cell growth. The cytoplasmic domain of β8 is divergent in sequence, lacking all amino acid homology with the highly homologous cytoplasmic domains of the other αv-associating integrin β subunits (β1, β3, β5, and β6). The 08 cytoplasmic domain is divergent in function. αvβ8 has a restricted distribution and is most highly expressed in nonproliferating cell types. The anti-Integrin αvβ8 antibodies as described in SEQ ID NO: 147-SEQ ID NO: 148 have been developed to target Integrin αvβ8.

CD248 encodes endosialin. Endosialin is a member of the “Group XIV”, a novel family of C-type lectin transmembrane receptors which play a role not only in cell-cell adhesion processes but also in host defense. Endosialin has been associated with angiogenesis in the embryo, uterus and in tumor development and growth. Monoclonal antibody Ontuxizumab has been developed to target endosialin.

FAP (fibroblast activation protein alpha) is a 170 kDa melanoma membrane-bound gelatinase, protein that in humans is encoded by the FAP gene. The protein encoded by this gene is a homodimeric integral membrane gelatinase belonging to the serine protease family. It is selectively expressed in reactive stromal fibroblasts of epithelial cancers, granulation tissue of healing wounds, and malignant cells of bone and soft tissue sarcomas. This protein is thought to be involved in the control of fibroblast growth or epithelial-mesenchymal interactions during development, tissue repair, and epithelial carcinogenesis. The anti-FAP antibodies as described in SEQ ID NO: 151-SEQ ID NO: 168 have been developed to target FAP.

Integrin αv subunit associates with one of five integrin β subunits, β1, β3, β5, β6, or β8, to form five distinct αVβ heterodimers. The integrin αVβ heterodimers on the cell surface interact with cell adhesive proteins, such as collagen, fibrinogen, fibronectin, and vitronectin. These interactions play an important role in cell adhesion or migration, especially in tumor metastasis. Monoclonal antibody intetumumab and anti-Integrin αv antibodies as described in SEQ ID NO: 171-SEQ ID NO: 174 have been developed to target Integrin αv.

Integrin αvβ6 is an epithelial-specific integrin that is a receptor for the extracellular matrix (ECM) proteins fibronectin, vitronectin, tenascin and the latency associated peptide (LAP) of TGF-β. Integrin αvβ6 is not expressed in healthy adult epithelia but is upregulated during wound healing and in cancer. Integrin αvβ6 has been shown to modulate invasion, inhibit apoptosis, regulate the expression of matrix metalloproteases (MMPs) and activate TGF-β1. The anti-Integrin αvβ6 antibodies as described in SEQ ID NO: 175-SEQ ID NO: 182 have been developed to target Integrin αvβ6.

### Antibody Constructs

Disclosed herein are antibody constructs and targeting moieties that may be used together with compounds of the disclosure. In certain embodiments, compounds of the disclosure are conjugated either directly or through a linker group to an antibody construct or a targeting moiety to form conjugates.

In certain embodiments, conjugates of the disclosure are represented by the following formula:

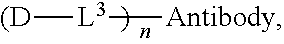

wherein Antibody is an antibody construct, L³ is a linker, D is a compound or salt disclosed herein, and n is from 1 to 20. In certain embodiments, n is from 1 to 10, such as from 1 to 9, such as from 1 to 8, such as from 2 to 8, such as from 1 to 6, such as from 3 to 5, or such as from 1 to 3. In certain embodiments, n is 4. In certain embodiments, each D is independently selected from Formulas (I-A), (I-B), (I-C), (I-D), (I-E), (II-A), (II-B), (II-C), and (II-D) and Table 14, respectively.

In certain embodiments, conjugates of the disclosure are represented by the following formula:

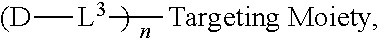

wherein L³ is a linker, D is a compound or salt disclosed herein, and n is from 1 to 20. In certain embodiments, n is from 1 to 10, such as from 1 to 9, such as from 1 to 8, such as from 2 to 8, such as from 1 to 6, such as from 3 to 5, or such as from 1 to 3. In certain embodiments, n is 4. In certain embodiments, each D is independently selected from Formulas (I-A), (I-B), (I-C), (I-D), (I-E), (II-A), (II-B), (II-C), and (II-D), and Table 14, respectively.

In certain embodiments, a compound or salt of the disclosure, e.g., a compound or salt of Formulas (I-A), (I-B), (I-C), (I-D), (I-E), (II-A), (II-B), (II-C), and (II-D), and Table 14, also may be referred to herein as a TGFβR2 inhibitor, a drug, D, an amino-pyrazinecarboxamide compound, or a payload, particularly when referenced as part of a conjugate. “LP”, “linker-payload”, “L³-D”, or “compound-linker” may be used herein to refer to a compound or salt of the disclosure bound to a linker.

An antibody construct may contain, for example, two, three, four, five, six, seven, eight, nine, ten, or more antigen binding domains. An antibody construct may contain two antigen binding domains in which each antigen binding domain can recognize the same antigen. An antibody construct may contain two antigen binding domains in which each antigen binding domain can recognize different antigens. An antigen binding domain may be in a scaffold, in which a scaffold is a supporting framework for the antigen binding domain. An antigen binding domain may be in a non-antibody scaffold. An antigen binding domain may be in an antibody scaffold. An antibody construct may comprise an antigen binding domain in a scaffold. The antibody construct may comprise an Fc fusion protein. In some embodiments, the antibody construct is an Fc fusion protein. An antigen binding domain may specifically bind to a tumor antigen. An antigen binding domain may specifically bind to an antigen having at least 80%, at least 90%, at least 95%, at least 99%, or 100% sequence identity to a tumor antigen. An antigen binding domain may specifically bind to an antigen on an antigen presenting cell (APC). An antigen binding domain may specifically bind to an antigen having at least 80%, at least 90%, at least 95%, at least 99%, or 100% sequence identity to an antigen on an antigen presenting cell (APC).

An antigen binding domain of an antibody may comprise one or more light chain (L) CDRs and one or more heavy chain (H) CDRs. For example, an antigen binding domain of an antibody may comprise one or more of the following: a light chain complementary determining region 1 (LCDR1), a light chain complementary determining region 2 (LCDR2), or a light chain complementary determining region 3 (LCDR3). For another example, an antigen binding domain may comprise one or more of the following: a heavy chain complementary determining region 1 (HCDR1), a heavy chain complementary determining region 2 (HCDR2), or a heavy chain complementary determining region 3 (HCDR3). As an additional example, an antigen binding domain of an antibody may comprise one or more of the following: LCDR1, LCDR2, LCDR3, HCDR1, HCDR2, and HCDR3. In some embodiments, an antigen binding domain of an antibody includes all six CDRs, (i.e., LCDR1, LCDR2, LCDR3, HCDR1, HCDR2, and HCDR3).

The antigen binding domain of an antibody construct may be selected from any domain that specifically binds the antigen including, but not limited to, from a monoclonal antibody, a polyclonal antibody, a recombinant antibody, or binding functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

The antigen binding domain of an antibody construct may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

In certain embodiments, an antibody construct of the disclosure comprises an Fc domain that may comprise an Fc region, in which the Fc domain may be the part of the Fc region that interacts with Fc receptors. The Fc domain of an antibody construct may interact with Fc-receptors (FcRs) found on immune cells. The Fc domain may also mediate the interaction between effector molecules and cells, which can lead to activation of the immune system. The Fc region may be derived from IgG, IgA, or IgD antibody isotypes, and may comprise two identical protein fragments, which are derived from the second and third constant domains of the antibody's heavy chains. In an Fc domain or region derived from an IgG antibody isotype, the Fc domain or region may comprise a highly-conserved N-glycosylation site, which may be essential for FcR-mediated downstream effects. The Fc domain or region may be derived from IgM or IgE antibody isotypes, in which the Fc domain or region may comprise three heavy chain constant domains.

An Fc domain may interact with different types of FcRs. The different types of FcRs may include, for example, FcγRI, FcγRIIA, FcγRIIB, FcγRIIIA, FcγRIIIB, FcαRI, FcμR, FcΠRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

In some embodiments, an Fc domain or region can exhibit reduced binding affinity to one or more Fc receptors. In some embodiments, an Fc domain or region can exhibit reduced binding affinity to one or more Fcgamma receptors. In some embodiments, an Fc domain or region can exhibit reduced binding affinity to FcRn receptors. In some embodiments, an Fc domain or region can exhibit reduced binding affinity to Fcgamm and FcRn receptors. In some embodiments, an Fc domain is an Fc null domain or region. As used herein, an “Fc null” refers to a domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

The Fc domain may have one or more, two or more, three or more, or four or more amino acid substitutions that decrease binding of the Fc domain to an Fc receptor. In certain embodiments, an Fc domain exhibits decreased binding to FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more amino acid substitutions that has the effect of reducing the affinity of the Fc domain or region to an Fc receptor. In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Kabat numbering.

In some embodiments, the Fc domain or region can comprise a sequence of the IgG1 isoform that has been modified from the wild-type IgG1 sequence. A modification can comprise a substitution at more than one amino acid residue, such as at 5 different amino acid residues including L235V/F243L/R292P/Y300L/P396L (IgG1VLPLL) according to the EU index of Kabat numbering. A modification can comprise a substitution at more than one amino acid residue such as at 2 different amino acid residues including S239D/1332E (IgG1DE) according to the EU index of Kabat numbering. A modification can comprise a substitution at more than one amino acid residue such as at 3 different amino acid residues including S298A/E333A/K334A (IgG1AAA) according to the EU index of Kabat numbering.

An antibody construct may consist of two identical light protein chains and two identical heavy protein chains, all held together covalently by disulfide linkages. The N-terminal regions of the light and heavy chains together may form the antigen recognition site of an antibody. Structurally, various functions of an antibody may be confined to discrete protein domains. The sites that can recognize and can bind antigen may consist of three complementarities determining regions (CDRs) that may lie within the variable heavy chain region and variable light chain region at the N-terminal end of the heavy chain and the light chain. The constant domains may provide the general framework of the antibody and may not be involved directly in binding the antibody to an antigen, but may be involved in various effector functions, such as participation of the antibody in antibody-dependent cellular cytotoxicity, and may bind Fc receptors. The constant domains may include an Fc region. The constant domains may include an Fe domain. The variable regions of natural light and heavy chains may have the same general structures, and each domain may comprise four framework regions, whose sequences can be somewhat conserved, connected by three hyper-variable regions or CDRs. The four framework regions (FR) may largely adopt a β-sheet conformation and the CDRs can form loops connecting, and in some aspects forming part of, the β-sheet structure. The CDRs in each chain may be held in close proximity by the framework regions and with the CDRs from the other chain, may contribute to the formation of the antigen binding site.

An antibody construct may comprise a light chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence. An antibody construct may comprise a heavy chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence.

An antibody construct may be an antibody. Antibodies may be selected from different classes of immunoglobins, e.g., IgA, IgD, IgE, IgG, and IgM. The several different classes may be further divided into isotypes, e.g., IgG1, IgG2, IgG3, IgG4, IgA1, and IgA2. An antibody may further comprise a light chain and a heavy chain, often more than one chain. The heavy-chain constant regions (Fc) that corresponds to the different classes of immunoglobulins may be α, δ, ε, γ, and μ, respectively. The light chains may be one of either kappa (κ) or lambda (λ), based on the amino acid sequences of the constant domains. The Fc domain may further comprise an Fc region. An Fc receptor may bind an Fc domain. Antibody constructs may also include any fragment or recombinant forms thereof, including but not limited to, single chain variable fragments (scFvs).

An antibody construct may comprise an antigen-binding antibody fragment. An antibody fragment may include (i) a Fab fragment, a monovalent fragment consisting of the V_{L}, V_{H}, CL and CHI domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules.

F(ab′)₂ and Fab′ moieties may be produced by genetic engineering or by treating immunoglobulin (e.g., monoclonal antibody) with a protease such as pepsin and papain, and may include an antibody fragment generated by digesting immunoglobulin near the disulfide bonds existing between the hinge regions in each of the two H chains. The Fab fragment may also contain the constant domain of the light chain and the first constant domain (C_{H1}) of the heavy chain. Fab′ fragments may differ from Fab fragments by the addition of a few residues at the carboxyl terminus of the heavy chain C_{H1} domain including one or more cysteine(s) from the antibody hinge region.

An Fv may be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) may recognize and bind to antigen, although the binding can be at a lower affinity than the affinity of the entire binding site.

An antibody construct may include an Fc domain comprising an Fc region or several Fc domains. The Fc domain of an antibody may interact with FcRs found on immune cells. The Fc domain may also mediate the interaction between effector molecules and cells, which may lead to activation of the immune system. In the IgG, IgA, and IgD antibody isotypes, the Fc region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

An antibody construct used herein may be “chimeric” or “humanized.” Chimeric and humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDRs correspond to those of a non-human immunoglobulin and all or substantially all of the framework regions are those of a human immunoglobulin sequence. The humanized antibody may also comprise at least a portion of an immunoglobulin constant region (Fc), typically that of a human immunoglobulin consensus sequence.

An antibody construct may be a human antibody. As used herein, “human antibodies” can include antibodies having, for example, the amino acid sequence of a human immunoglobulin and may include antibodies isolated from human immunoglobulin libraries or from animals transgenic for one or more human immunoglobulins that do not express endogenous immunoglobulins. Human antibodies may be produced using transgenic mice which are incapable of expressing functional endogenous immunoglobulins, but which may express human immunoglobulin genes. Completely human antibodies that recognize a selected epitope may be generated using guided selection. In this approach, a selected non-human monoclonal antibody, e.g., a mouse antibody, may be used to guide the selection of a completely human antibody recognizing the same epitope.

An antibody may be a bispecific antibody or a dual variable domain antibody (DVD). Bispecific and DVD antibodies may be monoclonal, often human or humanized, antibodies that can have binding specificities for at least two different antigens.

An antibody may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

An antibody may have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding may be reduced by, for example, mutating the immunoglobulin constant region segment of the antibody at particular regions necessary for FcR interactions.

An antibody or Fc domain may be modified to acquire or improve at least one constant region-mediated biological effector function relative to an unmodified antibody or Fc domain, e.g., to enhance FcγR interactions. For example, an antibody with a constant region that binds to FcγRIIA, FcγRIIB and/or FcγRIIIA with greater affinity than the corresponding wild type constant region may be produced according to the methods described herein. An Fc domain that binds to FcγRIIA, FcγRIIB and/or FcγRIIIA with greater affinity than the corresponding wild type Fc domain may be produced according to the methods described herein or known to the skilled artisan.

In certain embodiments, an antibody construct comprises an Fc domain that may comprise an Fc region, in which the Fc domain may be the part of the Fc region that interacts with Fc receptors. The Fc domain of an antibody construct may interact with Fc-receptors (FcRs) found on immune cells. The Fc domain may also mediate the interaction between effector molecules and cells, which can lead to activation of the immune system. The Fc region may be derived from IgG, IgA, or IgD antibody isotypes, and may comprise two identical protein fragments, which are derived from the second and third constant domains of the antibody's heavy chains. In an Fc domain or region derived from an IgG antibody isotype, the Fc domain or region may comprise a highly-conserved N-glycosylation site, which may be essential for FcR-mediated downstream effects. The Fc domain or region may be derived from IgM or IgE antibody isotypes, in which the Fc domain or region may comprise three heavy chain constant domains.

An Fc domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

In some embodiments, an Fc domain or region of the antibody construct portion of a conjugate can exhibit increased binding affinity to one or more Fc receptors. In some embodiments, an Fc domain or region can exhibit increased binding affinity to one or more Fcgamma receptors. In some embodiments, an Fc domain or region can exhibit increased binding affinity to FcRn receptors. In some embodiments, an Fc domain or region can exhibit increased binding affinity to Fcgamma and FcRn receptors.

In some embodiments, an Fc domain or region of the antibody construct portion of a conjugate can exhibit reduced binding affinity to one or more Fc receptors. In some embodiments, an Fc domain or region can exhibit reduced binding affinity to one or more Fcgamma receptors. In some embodiments, an Fc domain or region can exhibit reduced binding affinity to FcRn receptors. In some embodiments, an Fc domain or region can exhibit reduced binding affinity to Fcgamma and FcRn receptors. In some embodiments, an Fc domain is an Fc null domain or region. In some embodiments, an Fc domain or region can exhibit reduced binding affinity to FcRn receptors, but have the same or increased binding affinity to one or more Fcgamma receptors as compared to a wildtype IgG. In some embodiments, an Fc domain or region can exhibit increased binding affinity to FcRn receptors, but have the same or decreased binding affinity to one or more Fcgamma receptors.

The Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more amino acid substitutions that reduces the binding affinity of the Fc domain or region to an Fc receptor.

In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Kabat numbering.

In some embodiments, the Fc domain or region can comprise a sequence of an IgG isoform that has been modified from the wild-type IgG sequence. In some embodiments, the Fc domain or region can comprise a sequence of the IgG1 isoform that has been modified from the wild-type IgG1 sequence. In some embodiments, the modification comprises substitution of one or more amino acids that reduce binding affinity of an IgG Fc domain or region to all Fcγ receptors. A modification can be substitution of E233, L234 and L235, such as E233P/L234V/L235A or E233P/L234V/L235A/ΔG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain or region comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fe domain or region comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain or region to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain or region to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain or region to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc domain or region to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain or region to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain or region to FcγRII receptor and increases the binding affinity to FcγRIIIA receptor. A modification can be a substitution of S298, such as S298A, according to the EU index of Kabat. A modification can be substitution of S239, 1332 and A330, such as S239D/1332E/A330L. A modification can be substitution of S239 and 1332, such as S239D/1332E.

In some embodiments, the modification comprises substitution of one or more amino acids that decreases binding affinity of an IgG Fc domain or region to FcγRIIIA receptor. A modification can be substitution of F241 and F243, such as F241S/F243S or F241I/F243I, according to the EU index of Kabat.

In some embodiments, the modification comprises substitution of one or more amino acids that decreases binding affinity of an IgG Fc domain or region to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain or region to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and 1332, such as S239D/1332E.

In some embodiments, the modification comprises substitution of one or more amino acids that increases binding affinity of an IgG Fc domain or region to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of 5298, E333 and K334, such as S298A/E333A/K334A, according to the EU index of Kabat. A modification can be substitution of K246, such as K246F, according to the EU index of Kabat.

Other substitutions in an IgG Fc domain that affect its interaction with one or more Fcγ receptors are disclosed in U.S. Pat. Nos. 7,317,091 and 8,969,526 (the disclosures of which are incorporated by reference herein).

In some embodiments, an IgG Fc domain or region comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at I253, such as I253A according to the EU index of Kabat. A modification can comprise a substitution at H310, such as H310A according to the EU index of Kabat. A modification can comprise substitutions at I253, H310 and H435, such as 1253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q31V, D376V, and N434H. Other substitutions in an IgG Fe domain that affect its interaction with FcRn are disclosed in U.S. Pat. No. 9,803,023 (the disclosure of which is incorporated by reference herein).

In certain embodiments, the antibody construct comprises an antigen binding domain and an Fc domain.

In certain embodiments, the antigen binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen that is at least 80% identical to an antigen on a T cell, an APC, and/or a B cell. In certain embodiments, the antigen binding domain specifically binds to an antigen that is at least 80% identical to an antigen on a hepatocyte. In certain embodiments, the antigen binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1. In certain embodiments, the antigen binding domain may specifically bind to an antigen that is at least 80% identical to an antigen selected from the group consisting of ASGR1 and ASGR2 (asialoglycoprotein receptor 1 and 2). In certain embodiments, the antigen binding domain specifically binds to an antigen that is at least 80% identical to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen that is at least 80% identical to an antigen selected from the group consisting of PDGFRβ, integrin αvβ1, integrin αvβ3, integrin αvγ6, integrin αvβ8, Endosialin, FAP, ADAM12, LRRC15, MMP14, PDPN, CDH11 and F2RL2, In certain embodiments, the antigen binding domain may specifically bind to an antigen that is at least 80% identical to an antigen selected from the group consisting of FAP, ADAM12, LRRC15, MMP14, PDPN, CDH11 and F2RL2, In certain embodiments, the antigen binding domain specifically binds to an antigen that is at least 80% identical to an antigen on a tumor cell, a tumor antigen. In certain embodiments, the antigen binding domain specifically binds to an antigen that is at least 80% identical to an antigen selected from the group consisting of MUC16, UPKiB, VTCN1, TMPRSS3, TMEM238, Clorf186, TMPRSS4, CLDN6, CLDN8, STRA6, MSLN or CD73.

In certain embodiments, the antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen on a T cell, an APC, and/or a B cell. In certain embodiments, the antigen binding domain specifically binds to an antigen on a hepatocyte. In certain embodiments, the antigen binding domain may specifically bind to an antigen selected from the group consisting of CLTA4, PD-1, OX40, LAG-3, GITR, GARP, CD25, CD27, PD-L1, TNFR2, ICOS, 41BB, CD70, CD73, CD38 or VTCN1. certain embodiments, the antigen binding domain may specifically bind to an antigen selected from the group consisting of ASGR1 and ASGR2. In certain embodiments, the antigen binding domain specifically binds to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen selected from the group consisting of, PDGFRβ, integrin αvβ1, integrin αvβ3, integrin αvβ6, integrin αvβ8, Endosialin, FAP, ADAM12, LRRC15, MMP14, PDPN, CDH11 and F2RL2. In certain embodiments, the antigen binding domain may specifically bind to an antigen selected from the group consisting of FAP, ADAM12, LRRC15, MMP14, PDPN, CDH11 and F2RL2. In certain embodiments, the antigen is LRRC15. In certain embodiments, the antigen binding domain specifically binds to an antigen on a tumor cell, a tumor antigen. In certain embodiments, the antigen binding domain specifically binds to an antigen selected from the group consisting of MUC16, UPKiB, VTCN1, TMPRSS3, TMEM238, Clorf186, TMPRSS4, CLDN6, CLDN8, STRA6, MSLN or CD73.

An antibody construct may comprise an antibody with modifications of at least one amino acid residue. Modifications may be substitutions, additions, mutations, deletions, or the like. An antibody modification can be an insertion of an unnatural amino acid.

An antigen binding domain may comprise at least 80% sequence identity to any sequence in Table 1. An antigen binding domain may comprise a set of CDRs set forth in Table 1. An antibody construct may comprise an antigen binding domain that binds an antigen, wherein the antigen binding domain comprises at least at least 80%, at least 90%, at least 95%, at least 97%, at least 98%, at least 99%, or at least 100% sequence identity to: a) HCDR1 comprising an amino acid sequence of SEQ ID NO: 1, HCDR2 comprising an amino acid sequence of SEQ ID NO: 2, HCDR3 comprising an amino acid sequence of SEQ ID NO: 3, LCDR1 comprising an amino acid sequence of SEQ ID NO: 4, LCDR2 comprising an amino acid sequence of SEQ ID NO: 5, and LCDR3 comprising an amino acid sequence of SEQ ID NO: 6; b) HCDR1 comprising an amino acid sequence of SEQ ID NO: 7, HCDR2 comprising an amino acid sequence of SEQ ID NO: 8, HCDR3 comprising an amino acid sequence of SEQ ID NO: 9, LCDR1 comprising an amino acid sequence of SEQ ID NO: 10, LCDR2 comprising an amino acid sequence of SEQ ID NO: 11, and LCDR3 comprising an amino acid sequence of SEQ ID NO: 12; c) HCDR1 comprising an amino acid sequence of SEQ ID NO: 13, HCDR2 comprising an amino acid sequence of SEQ ID NO: 14, HCDR3 comprising an amino acid sequence of SEQ ID NO: 15, LCDR1 comprising an amino acid sequence of SEQ ID NO: 16, LCDR2 comprising an amino acid sequence of SEQ ID NO: 17, and LCDR3 comprising an amino acid sequence of SEQ ID NO: 18; d) HCDR1 comprising an amino acid sequence of SEQ ID NO: 19, HCDR2 comprising an amino acid sequence of SEQ ID NO: 20, HCDR3 comprising an amino acid sequence of SEQ ID NO: 21, LCDR1 comprising an amino acid sequence of SEQ ID NO: 22, LCDR2 comprising an amino acid sequence of SEQ ID NO: 23, and LCDR3 comprising an amino acid sequence of SEQ ID NO: 24; e) HCDR1 comprising an amino acid sequence of SEQ ID NO: 25, HCDR2 comprising an amino acid sequence of SEQ ID NO: 26, HCDR3 comprising an amino acid sequence of SEQ ID NO: 27, LCDR1 comprising an amino acid sequence of SEQ ID NO: 28, LCDR2 comprising an amino acid sequence of SEQ ID NO: 29, and LCDR3 comprising an amino acid sequence of SEQ ID NO: 30; f) HCDR1 comprising an amino acid sequence of SEQ ID NO: 31, HCDR2 comprising an amino acid sequence of SEQ ID NO: 32, HCDR3 comprising an amino acid sequence of SEQ ID NO: 33, LCDR1 comprising an amino acid sequence of SEQ ID NO: 34, LCDR2 comprising an amino acid sequence of SEQ ID NO: 35, and LCDR3 comprising an amino acid sequence of SEQ ID NO: 36; g) HCDR1 comprising an amino acid sequence of SEQ ID NO: 37, HCDR2 comprising an amino acid sequence of SEQ ID NO: 38, HCDR3 comprising an amino acid sequence of SEQ ID NO: 39, LCDR1 comprising an amino acid sequence of SEQ ID NO: 40, LCDR2 comprising an amino acid sequence of SEQ ID NO: 41, and LCDR3 comprising an amino acid sequence of SEQ ID NO: 42; h) HCDR1 comprising an amino acid sequence of SEQ ID NO: 43, HCDR2 comprising an amino acid sequence of SEQ ID NO: 44, HCDR3 comprising an amino acid sequence of SEQ ID NO: 45, LCDR1 comprising an amino acid sequence of SEQ ID NO: 46, LCDR2 comprising an amino acid sequence of SEQ ID NO: 47, and LCDR3 comprising an amino acid sequence of SEQ ID NO: 48; i) HCDR1 comprising an amino acid sequence of SEQ ID NO: 49, HCDR2 comprising an amino acid sequence of SEQ ID NO: 50, HCDR3 comprising an amino acid sequence of SEQ ID NO: 51, LCDR1 comprising an amino acid sequence of SEQ ID NO: 52, LCDR2 comprising an amino acid sequence of SEQ ID NO: 53, and LCDR3 comprising an amino acid sequence of SEQ ID NO: 54; j) HCDR1 comprising an amino acid sequence of SEQ ID NO: 55, HCDR2 comprising an amino acid sequence of SEQ ID NO: 56, HCDR3 comprising an amino acid sequence of SEQ ID NO: 57, LCDR1 comprising an amino acid sequence of SEQ ID NO: 58, LCDR2 comprising an amino acid sequence of SEQ ID NO: 59, and LCDR3 comprising an amino acid sequence of SEQ ID NO: 60; k) HCDR1 comprising an amino acid sequence of SEQ ID NO: 61, HCDR2 comprising an amino acid sequence of SEQ ID NO: 62, HCDR3 comprising an amino acid sequence of SEQ ID NO: 63, LCDR1 comprising an amino acid sequence of SEQ ID NO: 64, LCDR2 comprising an amino acid sequence of SEQ ID NO: 65, and LCDR3 comprising an amino acid sequence of SEQ ID NO: 66; 1) HCDR1 comprising an amino acid sequence of SEQ ID NO: 67, HCDR2 comprising an amino acid sequence of SEQ ID NO: 68, HCDR3 comprising an amino acid sequence of SEQ ID NO: 69, LCDR1 comprising an amino acid sequence of SEQ ID NO: 70, LCDR2 comprising an amino acid sequence of SEQ ID NO: 71, and LCDR3 comprising an amino acid sequence of SEQ ID NO: 72; m) HCDR1 comprising an amino acid sequence of SEQ ID NO: 73, HCDR2 comprising an amino acid sequence of SEQ ID NO: 74, HCDR3 comprising an amino acid sequence of SEQ ID NO: 75, LCDR1 comprising an amino acid sequence of SEQ ID NO: 76, LCDR2 comprising an amino acid sequence of SEQ ID NO: 77, and LCDR3 comprising an amino acid sequence of SEQ ID NO: 78; n) HCDR1 comprising an amino acid sequence of SEQ ID NO: 73, HCDR2 comprising an amino acid sequence of SEQ ID NO: 74, HCDR3 comprising an amino acid sequence of SEQ ID NO: 75, LCDR1 comprising an amino acid sequence of SEQ ID NO: 79, LCDR2 comprising an amino acid sequence of SEQ ID NO: 80, and LCDR3 comprising an amino acid sequence of SEQ ID NO: 81; o) HCDR1 comprising an amino acid sequence of SEQ ID NO: 199, HCDR2 comprising an amino acid sequence of SEQ ID NO: 200, HCDR3 comprising an amino acid sequence of SEQ ID NO: 201, LCDR1 comprising an amino acid sequence of SEQ ID NO: 202, LCDR2 comprising an amino acid sequence of SEQ ID NO: 203, and LCDR3 comprising an amino acid sequence of SEQ ID NO: 204; p) HCDR1 comprising an amino acid sequence of SEQ ID NO: 205, HCDR2 comprising an amino acid sequence of SEQ ID NO: 206, HCDR3 comprising an amino acid sequence of SEQ ID NO: 207, LCDR1 comprising an amino acid sequence of SEQ ID NO: 208, LCDR2 comprising an amino acid sequence of SEQ ID NO: 209, and LCDR3 comprising an amino acid sequence of SEQ ID NO: 210 q) HCDR1 comprising an amino acid sequence of SEQ ID NO: 211, HCDR2 comprising an amino acid sequence of SEQ ID NO: 212, HCDR3 comprising an amino acid sequence of SEQ ID NO: 213, LCDR1 comprising an amino acid sequence of SEQ ID NO: 214, LCDR2 comprising an amino acid sequence of SEQ ID NO: 215, and LCDR3 comprising an amino acid sequence of SEQ ID NO: 216 r) HCDR1 comprising an amino acid sequence of SEQ ID NO: 217, HCDR2 comprising an amino acid sequence of SEQ ID NO: 218, HCDR3 comprising an amino acid sequence of SEQ ID NO: 219, LCDR1 comprising an amino acid sequence of SEQ ID NO: 220, LCDR2 comprising an amino acid sequence of SEQ ID NO: 221, and LCDR3 comprising an amino acid sequence of SEQ ID NO: 222; s) HCDR1 comprising an amino acid sequence of SEQ ID NO: 223, HCDR2 comprising an amino acid sequence of SEQ ID NO: 224, HCDR3 comprising an amino acid sequence of SEQ ID NO: 225, LCDR1 comprising an amino acid sequence of SEQ ID NO: 226, LCDR2 comprising an amino acid sequence of SEQ ID NO: 227, and LCDR3 comprising an amino acid sequence of SEQ ID NO: 228; or t) HCDR1 comprising an amino acid sequence of SEQ ID NO: 229, HCDR2 comprising an amino acid sequence of SEQ ID NO: 230, HCDR3 comprising an amino acid sequence of SEQ ID NO: 231, LCDR1 comprising an amino acid sequence of SEQ ID NO: 232, LCDR2 comprising an amino acid sequence of SEQ ID NO: 233, and LCDR3 comprising an amino acid sequence of SEQ ID NO: 234.

An antibody construct may comprise an antigen binding domain comprising one or more variable domains. An antibody construct may comprise an antigen binding domain comprising a light chain variable domain (V_{L} domain). A binding domain may comprise at least 80%, at least 90%, at least 95%, at least 97%, at least 98%, at least 99%, or at least 100% sequence identity to any V_{L} sequence in Table 2. An antibody construct may comprise an antigen binding domain comprising a heavy chain variable domain (V_{H} domain). An antigen binding domain may comprise at least 80%, at least 90%, at least 95%, at least 97%, at least 98%, at least 99%, or at least 100% sequence identity to any V_{H} sequence in Table 2. An antigen binding domain can comprise a pair of V_{H} and V_{L} sequences in Table 2. An antigen binding domain can comprise at least 80% sequence identity to any sequence in Table 2.

An antibody construct may comprise an antigen binding domain that specifically binds an antigen, wherein the antigen binding domain comprises: a) a V_{H} sequence having at least 80%, at least 90%, at least 95%, at least 97%, at least 98%, at least 99%, or at least 100% sequence identity to an amino acid sequence of SEQ ID NO: 83, and a V_{L} sequence having at least 80%, at least 90%, at least 95%, at least 97%, at least 98%, at least 99%, or at least 100% sequence identity to an amino acid sequence of SEQ ID NO: 84; b) a V_{H} sequence having at least 80%, at least 90%, at least 95%, at least 97%, at least 98%, at least 99%, or at least 100% sequence identity to an amino acid sequence of SEQ ID NO: 85, and a V_{L} sequence having at least 80%, at least 90%, at least 95%, at least 97%, at least 98%, at least 99%, or at least 100% sequence identity to an amino acid sequence of SEQ ID NO: 86; c) a V_{H} sequence having at least 80%, at least 90%, at least 95%, at least 97%, at least 98%, at least 99%, or at least 100% sequence identity to an amino acid sequence of SEQ ID NO: 87, and a V_{L} sequence having at least 80%, at least 90%, at least 95%, at least 97%, at least 98%, at least 99%, or at least 100% sequence identity to an amino acid sequence of SEQ ID NO: 88; d) a V_{H} sequence having at least 80%, at least 90%, at least 95%, at least 97%, at least 98%, at least 99%, or at least 100% sequence identity to an amino acid sequence of SEQ ID NO: 89, and a V_{L} sequence having at least 80%, at least 90%, at least 95%, at least 97%, at least 98%, at least 99%, or at least 100% sequence identity to an amino acid sequence of SEQ ID NO: 90; e) a V_{H} sequence having at least 80%, at least 90%, at least 95%, at least 97%, at least 98%, at least 99%, or at least 100% sequence identity to an amino acid sequence of SEQ ID NO: 91, and a V_{L} sequence having at least 80%, at least 90%, at least 95%, at least 97%, at least 98%, at least 99%, or at least 100% sequence identity to an amino acid sequence of SEQ ID NO: 92; f) a V_{H} sequence having at least 80%, at least 90%, at least 95%, at least 97%, at least 98%, at least 99%, or at least 100% sequence identity to an amino acid sequence of SEQ ID NO: 93, and a V_{L} sequence having at least 80%, at least 90%, at least 95%, at least 97%, at least 98%, at least 99%, or at least 100% sequence identity to an amino acid sequence of SEQ ID NO: 94; g) a V_{H} sequence having at least 80%, at least 90%, at least 95%, at least 97%, at least 98%, at least 99%, or at least 100% sequence identity to an amino acid sequence of SEQ ID NO: 95, and a V_{L} sequence having at least 80%, at least 90%, at least 95%, at least 97%, at least 98%, at least 99%, or at least 100% sequence identity to an amino acid sequence of SEQ ID NO: 96; h) a V_{H} sequence having at least 80%, at least 90%, at least 95%, at least 97%, at least 98%, at least 99%, or at least 100% sequence identity to an amino acid sequence of SEQ ID NO: 97, and a V_{L} sequence having at least 80%, at least 90%, at least 95%, at least 97%, at least 98%, at least 99%, or at least 100% sequence identity to an amino acid sequence of SEQ ID NO: 98; i) a V_{H} sequence having at least 80%, at least 90%, at least 95%, at least 97%, at least 98%, at least 99%, or at least 100% sequence identity to an amino acid sequence of SEQ ID NO: 99, and a V_{L} sequence having at least 80%, at least 90%, at least 95%, at least 97%, at least 98%, at least 99%, or at least 100% sequence identity to an amino acid sequence of SEQ ID NO: 100; j) a V_{H} sequence having at least 80%, at least 90%, at least 95%, at least 97%, at least 98%, at least 99%, or at least 100% sequence identity to an amino acid sequence of SEQ ID NO: 101, and a V_{L} sequence having at least 80%, at least 90%, at least 95%, at least 97%, at least 98%, at least 99%, or at least 100% sequence identity to an amino acid sequence of SEQ ID NO: 102; k) a V_{H} sequence having at least 80%, at least 90%, at least 95%, at least 97%, at least 98%, at least 99%, or at least 100% sequence identity to an amino acid sequence of SEQ ID NO: 101, and a V_{L} sequence having at least 80%, at least 90%, at least 95%, at least 97%, at least 98%, at least 99%, or at least 100% sequence identity to an amino acid sequence of SEQ ID NO: 103; 1) a V_{H} sequence having at least 80%, at least 90%, at least 95%, at least 97%, at least 98%, at least 99%, or at least 100% sequence identity to an amino acid sequence of SEQ ID NO: 104, and a V_{L} sequence having at least 80%, at least 90%, at least 95%, at least 97%, at least 98%, at least 99%, or at least 100% sequence identity to an amino acid sequence of SEQ ID NO: 105; m) a V_{H} sequence having at least 80%, at least 90%, at least 95%, at least 97%, at least 98%, at least 99%, or at least 100% sequence identity to an amino acid sequence of SEQ ID NO: 106, and a V_{L} sequence having at least 80%, at least 90%, at least 95%, at least 97%, at least 98%, at least 99%, or at least 100% sequence identity to an amino acid sequence of SEQ ID NO: 107; n) a V_{H} sequence having at least 80%, at least 90%, at least 95%, at least 97%, at least 98%, at least 99%, or at least 100% sequence identity to an amino acid sequence of SEQ ID NO: 109, and a V_{L} sequence having at least 80%, at least 90%, at least 95%, at least 97%, at least 98%, at least 99%, or at least 100% sequence identity to an amino acid sequence of SEQ ID NO: 108; o) a V_{H} sequence having at least 80%, at least 90%, at least 95%, at least 97%, at least 98%, at least 99%, or at least 100% sequence identity to an amino acid sequence of SEQ ID NO: 110, and a V_{L} sequence having at least 80%, at least 90%, at least 95%, at least 97%, at least 98%, at least 99%, or at least 100% sequence identity to an amino acid sequence of SEQ ID NO: 108; p) a V_{H} sequence having at least 80%, at least 90%, at least 95%, at least 97%, at least 98%, at least 99%, or at least 100% sequence identity to an amino acid sequence of SEQ ID NO: 111, and a V_{L} sequence having at least 80%, at least 90%, at least 95%, at least 97%, at least 98%, at least 99%, or at least 100% sequence identity to an amino acid sequence of SEQ ID NO: 112; q) a V_{H} sequence having at least 80%, at least 90%, at least 95%, at least 97%, at least 98%, at least 99%, or at least 100% sequence identity to an amino acid sequence of SEQ ID NO: 113, and a V_{L} sequence having at least 80%, at least 90%, at least 95%, at least 97%, at least 98%, at least 99%, or at least 100% sequence identity to an amino acid sequence of SEQ ID NO: 114; r) a V_{H} sequence having at least 80%, at least 90%, at least 95%, at least 97%, at least 98%, at least 99%, or at least 100% sequence identity to an amino acid sequence of SEQ ID NO: 115, and a V_{L} sequence having at least 80%, at least 90%, at least 95%, at least 97%, at least 98%, at least 99%, or at least 100% sequence identity to an amino acid sequence of SEQ ID NO: 116; s) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 118; t) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 119; u) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 120; v) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 121; w) a V_{H} sequence having at least 80%, at least 90%, at least 95%, at least 97%, at least 98%, at least 99%, or at least 100% sequence identity to an amino acid sequence of SEQ ID NO: 117, and a V_{L} sequence having at least 80%, at least 90%, at least 95%, at least 97%, at least 98%, at least 99%, or at least 100% sequence identity to an amino acid sequence of SEQ ID NO: 122; x) a V_{H} sequence having at least 80%, at least 90%, at least 95%, at least 97%, at least 98%, at least 99%, or at least 100% sequence identity to an amino acid sequence of SEQ ID NO: 123, and a V_{L} sequence having at least 80%, at least 90%, at least 95%, at least 97%, at least 98%, at least 99%, or at least 100% sequence identity to an amino acid sequence of SEQ ID NO: 124; y) a V_{H} sequence having at least 80%, at least 90%, at least 95%, at least 97%, at least 98%, at least 99%, or at least 100% sequence identity to an amino acid sequence of SEQ ID NO: 125, and a V_{L} sequence having at least 80%, at least 90%, at least 95%, at least 97%, at least 98%, at least 99%, or at least 100% sequence identity to an amino acid sequence of SEQ ID NO: 126; z) a V_{H} sequence having at least 80%, at least 90%, at least 95%, at least 97%, at least 98%, at least 99%, or at least 100% sequence identity to an amino acid sequence of SEQ ID NO: 127, and a V_{L} sequence having at least 80%, at least 90%, at least 95%, at least 97%, at least 98%, at least 99%, or at least 100% sequence identity to an amino acid sequence of SEQ ID NO: 128; aa) a V_{H} sequence having at least 80%, at least 90%, at least 95%, at least 97%, at least 98%, at least 99%, or at least 100% sequence identity to an amino acid sequence of SEQ ID NO: 130, and a V_{L} sequence having at least 80%, at least 90%, at least 95%, at least 97%, at least 98%, at least 99%, or at least 100% sequence identity to an amino acid sequence of SEQ ID NO: 129; bb) a V_{H} sequence having at least 80%, at least 90%, at least 95%, at least 97%, at least 98%, at least 99%, or at least 100% sequence identity to an amino acid sequence of SEQ ID NO: 131, and a V_{L} sequence having at least 80%, at least 90%, at least 95%, at least 97%, at least 98%, at least 99%, or at least 100% sequence identity to an amino acid sequence of SEQ ID NO: 132; cc) a V_{H} sequence having at least 80%, at least 90%, at least 95%, at least 97%, at least 98%, at least 99%, or at least 100% sequence identity to an amino acid sequence of SEQ ID NO: 133, and a V_{L} sequence having at least 80%, at least 90%, at least 95%, at least 97%, at least 98%, at least 99%, or at least 100% sequence identity to an amino acid sequence of SEQ ID NO: 134; dd) a V_{H} sequence having at least 80%, at least 90%, at least 95%, at least 97%, at least 98%, at least 99%, or at least 100% sequence identity to an amino acid sequence of SEQ ID NO: 135, and a V_{L} sequence having at least 80%, at least 90%, at least 95%, at least 97%, at least 98%, at least 99%, or at least 100% sequence identity to an amino acid sequence of SEQ ID NO: 136; ee) a V_{H} sequence having at least 80%, at least 90%, at least 95%, at least 97%, at least 98%, at least 99%, or at least 100% sequence identity to an amino acid sequence of SEQ ID NO: 137, and a V_{L} sequence having at least 80%, at least 90%, at least 95%, at least 97%, at least 98%, at least 99%, or at least 100% sequence identity to an amino acid sequence of SEQ ID NO: 138; ff) a V_{H} sequence having at least 80%, at least 90%, at least 95%, at least 97%, at least 98%, at least 99%, or at least 100% sequence identity to an amino acid sequence of SEQ ID NO: 140, and a V_{L} sequence having at least 80%, at least 90%, at least 95%, at least 97%, at least 98%, at least 99%, or at least 100% sequence identity to an amino acid sequence of SEQ ID NO: 139; gg) a V_{H} sequence having at least 80%, at least 90%, at least 95%, at least 97%, at least 98%, at least 99%, or at least 100% sequence identity to an amino acid sequence of SEQ ID NO: 141, and a V_{L} sequence having at least 80%, at least 90%, at least 95%, at least 97%, at least 98%, at least 99%, or at least 100% sequence identity to an amino acid sequence of SEQ ID NO: 142; hh) a V_{H} sequence having at least 80%, at least 90%, at least 95%, at least 97%, at least 98%, at least 99%, or at least 100% sequence identity to an amino acid sequence of SEQ ID NO: 143, and a V_{L} sequence having at least 80%, at least 90%, at least 95%, at least 97%, at least 98%, at least 99%, or at least 100% sequence identity to an amino acid sequence of SEQ ID NO: 144; ii) a V_{H} sequence having at least 80%, at least 90%, at least 95%, at least 97%, at least 98%, at least 99%, or at least 100% sequence identity to an amino acid sequence of SEQ ID NO: 145, and a V_{L} sequence having at least 80%, at least 90%, at least 95%, at least 97%, at least 98%, at least 99%, or at least 100% sequence identity to an amino acid sequence of SEQ ID NO: 146; jj) a V_{H} sequence having at least 80%, at least 90%, at least 95%, at least 97%, at least 98%, at least 99%, or at least 100% sequence identity to an amino acid sequence of SEQ ID NO: 147, and a V_{L} sequence having at least 80%, at least 90%, at least 95%, at least 97%, at least 98%, at least 99%, or at least 100% sequence identity to an amino acid sequence of SEQ ID NO: 148; kk) a V_{H} sequence having at least 80%, at least 90%, at least 95%, at least 97%, at least 98%, at least 99%, or at least 100% sequence identity to an amino acid sequence of SEQ ID NO: 149, and a V_{L} sequence having at least 80%, at least 90%, at least 95%, at least 97%, at least 98%, at least 99%, or at least 100% sequence identity to an amino acid sequence of SEQ ID NO: 150; ll) a V_{H} sequence having at least 80%, at least 90%, at least 95%, at least 97%, at least 98%, at least 99%, or at least 100% sequence identity to an amino acid sequence of SEQ ID NO: 151, and a V_{L} sequence having at least 80%, at least 90%, at least 95%, at least 97%, at least 98%, at least 99%, or at least 100% sequence identity to an amino acid sequence of SEQ ID NO: 153; mm) a V_{H} sequence having at least 80%, at least 90%, at least 95%, at least 97%, at least 98%, at least 99%, or at least 100% sequence identity to an amino acid sequence of SEQ ID NO: 152, and a V_{L} sequence having at least 80%, at least 90%, at least 95%, at least 97%, at least 98%, at least 99%, or at least 100% sequence identity to an amino acid sequence of SEQ ID NO: 153; nn) a V_{H} sequence having at least 80%, at least 90%, at least 95%, at least 97%, at least 98%, at least 99%, or at least 100% sequence identity to an amino acid sequence of SEQ ID NO: 154, and a V_{L} sequence having at least 80%, at least 90%, at least 95%, at least 97%, at least 98%, at least 99%, or at least 100% sequence identity to an amino acid sequence of SEQ ID NO: 155; oo) a V_{H} sequence having at least 80%, at least 90%, at least 95%, at least 97%, at least 98%, at least 99%, or at least 100% sequence identity to an amino acid sequence of SEQ ID NO: 156, and a V_{L} sequence having at least 80%, at least 90%, at least 95%, at least 97%, at least 98%, at least 99%, or at least 100% sequence identity to an amino acid sequence of SEQ ID NO: 157; pp) a V_{H} sequence having at least 80%, at least 90%, at least 95%, at least 97%, at least 98%, at least 99%, or at least 100% sequence identity to an amino acid sequence of SEQ ID NO: 158, and a V_{L} sequence having at least 80%, at least 90%, at least 95%, at least 97%, at least 98%, at least 99%, or at least 100% sequence identity to an amino acid sequence of SEQ ID NO: 159; qq) a V_{H} sequence having at least 80%, at least 90%, at least 95%, at least 97%, at least 98%, at least 99%, or at least 100% sequence identity to an amino acid sequence of SEQ ID NO: 160, and a V_{L} sequence having at least 80%, at least 90%, at least 95%, at least 97%, at least 98%, at least 99%, or at least 100% sequence identity to an amino acid sequence of SEQ ID NO: 161; rr) a V_{H} sequence having at least 80%, at least 90%, at least 95%, at least 97%, at least 98%, at least 99%, or at least 100% sequence identity to an amino acid sequence of SEQ ID NO: 162, and a V_{L} sequence having at least 80%, at least 90%, at least 95%, at least 97%, at least 98%, at least 99%, or at least 100% sequence identity to an amino acid sequence of SEQ ID NO: 163; ss) a V_{H} sequence having at least 80%, at least 90%, at least 95%, at least 97%, at least 98%, at least 99%, or at least 100% sequence identity to an amino acid sequence of SEQ ID NO: 164, and a V_{L} sequence having at least 80%, at least 90%, at least 95%, at least 97%, at least 98%, at least 99%, or at least 100% sequence identity to an amino acid sequence of SEQ ID NO: 167; tt) a V_{H} sequence having at least 80%, at least 90%, at least 95%, at least 97%, at least 98%, at least 99%, or at least 100% sequence identity to an amino acid sequence of SEQ ID NO: 164, and a V_{L} sequence having at least 80%, at least 90%, at least 95%, at least 97%, at least 98%, at least 99%, or at least 100% sequence identity to an amino acid sequence of SEQ ID NO: 168; uu) a V_{H} sequence having at least 80%, at least 90%, at least 95%, at least 97%, at least 98%, at least 99%, or at least 100% sequence identity to an amino acid sequence of SEQ ID NO: 165, and a V_{L} sequence having at least 80%, at least 90%, at least 95%, at least 97%, at least 98%, at least 99%, or at least 100% sequence identity to an amino acid sequence of SEQ ID NO: 167; vv) a V_{H} sequence having at least 80%, at least 90%, at least 95%, at least 97%, at least 98%, at least 99%, or at least 100% sequence identity to an amino acid sequence of SEQ ID NO: 165, and a V_{L} sequence having at least 80%, at least 90%, at least 95%, at least 97%, at least 98%, at least 99%, or at least 100% sequence identity to an amino acid sequence of SEQ ID NO: 168; ww) a V_{H} sequence having at least 80%, at least 90%, at least 95%, at least 97%, at least 98%, at least 99%, or at least 100% sequence identity to an amino acid sequence of SEQ ID NO: 166, and a V_{L} sequence having at least 80%, at least 90%, at least 95%, at least 97%, at least 98%, at least 99%, or at least 100% sequence identity to an amino acid sequence of SEQ ID NO: 167; xx) a V_{H} sequence having at least 80%, at least 90%, at least 95%, at least 97%, at least 98%, at least 99%, or at least 100% sequence identity to an amino acid sequence of SEQ ID NO: 166, and a V_{L} sequence having at least 80%, at least 90%, at least 95%, at least 97%, at least 98%, at least 99%, or at least 100% sequence identity to an amino acid sequence of SEQ ID NO: 168; yy) a V_{H} sequence having at least 80%, at least 90%, at least 95%, at least 97%, at least 98%, at least 99%, or at least 100% sequence identity to an amino acid sequence of SEQ ID NO: 169, and a V_{L} sequence having at least 80%, at least 90%, at least 95%, at least 97%, at least 98%, at least 99%, or at least 100% sequence identity to an amino acid sequence of SEQ ID NO:170; zz) a V_{H} sequence having at least 80%, at least 90%, at least 95%, at least 97%, at least 98%, at least 99%, or at least 100% sequence identity to an amino acid sequence of SEQ ID NO: 171, and a V_{L} sequence having at least 80%, at least 90%, at least 95%, at least 97%, at least 98%, at least 99%, or at least 100% sequence identity to an amino acid sequence of SEQ ID NO: 172; aaa) a V_{H} sequence having at least 80%, at least 90%, at least 95%, at least 97%, at least 98%, at least 99%, or at least 100% sequence identity to an amino acid sequence of SEQ ID NO: 174, and a V_{L} sequence having at least 80%, at least 90%, at least 95%, at least 97%, at least 98%, at least 99%, or at least 100% sequence identity to an amino acid sequence of SEQ ID NO: 173; bbb) a V_{H} sequence having at least 80%, at least 90%, at least 95%, at least 97%, at least 98%, at least 99%, or at least 100% sequence identity to an amino acid sequence of SEQ ID NO: 175, and a V_{L} sequence having at least 80%, at least 90%, at least 95%, at least 97%, at least 98%, at least 99%, or at least 100% sequence identity to an amino acid sequence of SEQ ID NO: 176; ccc) a V_{H} sequence having at least 80%, at least 90%, at least 95%, at least 97%, at least 98%, at least 99%, or at least 100% sequence identity to an amino acid sequence of SEQ ID NO: 177, and a V_{L} sequence having at least 80%, at least 90%, at least 95%, at least 97%, at least 98%, at least 99%, or at least 100% sequence identity to an amino acid sequence of SEQ ID NO: 178; ddd) a V_{H} sequence having at least 80%, at least 90%, at least 95%, at least 97%, at least 98%, at least 99%, or at least 100% sequence identity to an amino acid sequence of SEQ ID NO: 179, and a V_{L} sequence having at least 80%, at least 90%, at least 95%, at least 97%, at least 98%, at least 99%, or at least 100% sequence identity to an amino acid sequence of SEQ ID NO: 180; eee) a V_{H} sequence having at least 80%, at least 90%, at least 95%, at least 97%, at least 98%, at least 99%, or at least 100% sequence identity to an amino acid sequence of SEQ ID NO: 181, and a V_{L} sequence having at least 80%, at least 90%, at least 95%, at least 97%, at least 98%, at least 99%, or at least 100% sequence identity to an amino acid sequence of SEQ ID NO: 182; fff) a V_{H} sequence having at least 80%, at least 90%, at least 95%, at least 97%, at least 98%, at least 99%, or at least 100% sequence identity to an amino acid sequence of SEQ ID NO: 183, and a V_{L} sequence having at least 80%, at least 90%, at least 95%, at least 97%, at least 98%, at least 99%, or at least 100% sequence identity to an amino acid sequence of SEQ ID NO: 184; ggg) a V_{H} sequence having at least 80%, at least 90%, at least 95%, at least 97%, at least 98%, at least 99%, or at least 100% sequence identity to an amino acid sequence of SEQ ID NO: 185, and a V_{L} sequence having at least 80%, at least 90%, at least 95%, at least 97%, at least 98%, at least 99%, or at least 100% sequence identity to an amino acid sequence of SEQ ID NO: 186; hhh) a V_{H} sequence having at least 80%, at least 90%, at least 95%, at least 97%, at least 98%, at least 99%, or at least 100% sequence identity to an amino acid sequence of SEQ ID NO: 187, and a V_{L} sequence having at least 80%, at least 90%, at least 95%, at least 97%, at least 98%, at least 99%, or at least 100% sequence identity to an amino acid sequence of SEQ ID NO: 188; iii) a V_{H} sequence having at least 80%, at least 90%, at least 95%, at least 97%, at least 98%, at least 99%, or at least 100% sequence identity to an amino acid sequence of SEQ ID NO: 189, and a V_{L} sequence having at least 80%, at least 90%, at least 95%, at least 97%, at least 98%, at least 99%, or at least 100% sequence identity to an amino acid sequence of SEQ ID NO: 190; jj) a V_{H} sequence having at least 80%, at least 90%, at least 95%, at least 97%, at least 98%, at least 99%, or at least 100% sequence identity to an amino acid sequence of SEQ ID NO: 191, and a V_{L} sequence having at least 80%, at least 90%, at least 95%, at least 97%, at least 98%, at least 99%, or at least 100% sequence identity to an amino acid sequence of SEQ ID NO: 192; kkk) a V_{H} sequence having at least 80%, at least 90%, at least 95%, at least 97%, at least 98%, at least 99%, or at least 100% sequence identity to an amino acid sequence of SEQ ID NO: 193, and a V_{L} sequence having at least 80%, at least 90%, at least 95%, at least 97%, at least 98%, at least 99%, or at least 100% sequence identity to an amino acid sequence of SEQ ID NO: 194; lll) a V_{H} sequence having at least 80%, at least 90%, at least 95%, at least 97%, at least 98%, at least 99%, or at least 100% sequence identity to an amino acid sequence of SEQ ID NO: 195, and a V_{L} sequence having at least 80%, at least 90%, at least 95%, at least 97%, at least 98%, at least 99%, or at least 100% sequence identity to an amino acid sequence of SEQ ID NO: 196; or mmm) a V_{H} sequence having at least 80%, at least 90%, at least 95%, at least 97%, at least 98%, at least 99%, or at least 100% sequence identity to an amino acid sequence of SEQ ID NO: 197, and a V_{L} sequence having at least 80%, at least 90%, at least 95%, at least 97%, at least 98%, at least 99%, or at least 100% sequence identity to an amino acid sequence of SEQ ID NO: 198.

An antibody construct may comprise a sequence from Table 1 and/or Table 2. An antibody construct may comprise a set of CDR sequences from Table 1 and/or a pair of V_{H} and V_{L} sequences from Table 2.

TABLE 1
Antibody CDRs

| ANTIBODY | REGION | SEQ ID NO: | SEQUENCE: |
|---|---|---|---|
| Ipilumumab | HCDR1 | 1 | GFTFSSYT |
| | HCDR2 | 2 | ISYDGNNK |
| | HCDR3 | 3 | ARTGWLGPFDY |
| | LCDR1 | 4 | QSVGSSY |
| | LCDR2 | 5 | SSY |
| | LCDR3 | 6 | QQYGSSPWT |
| Opdivo ® | HCDR1 | 7 | GITFSNSG |
| (nivolumab) | HCDR2 | 8 | IWYDGSKR |
| | HCDR3 | 9 | ATNDDY |
| | LCDR1 | 10 | QSVSSYL |
| | LCDR2 | 11 | DAS |
| | LCDR3 | 12 | QQSSNWPRT |
| Keytruda ® | HCDR1 | 13 | GYTFTNYY |
| (pembrolizumab) | HCDR2 | 14 | INPSNGGT |
| | HCDR3 | 15 | ARRDYRFDMGFDY |
| | LCDR1 | 16 | KGVSTSGYSY |
| | LCDR2 | 17 | LAS |
| | LCDR3 | 18 | QHSRDLPLT |
| Vonlerolizumab | HCDR1 | 19 | GYTFTDSY |
| | HCDR2 | 20 | MYPDNGDS |
| | HCDR3 | 21 | VLAPRWYFSV |
| | LCDR1 | 22 | QDISNY |
| | LCDR2 | 23 | YTS |
| | LCDR3 | 24 | QQGHTLPPT |
| Varlilumab | HCDR1 | 25 | GFTFSSYD |
| | HCDR2 | 26 | IWYDGSNK |
| | HCDR3 | 27 | ARGSGNWGFFDY |
| | LCDR1 | 28 | QGISRW |
| | LCDR2 | 29 | AAS |
| | LCDR3 | 30 | QQYNTYPRT |
| Zinbryta ® | HCDR1 | 31 | GYTFTSYR |
| (Daclizumab) | HCDR2 | 32 | INPSTGYT |
| | HCDR3 | 33 | ARGGGVFDY |
| | LCDR1 | 34 | SSSISY |
| | LCDR2 | 35 | TTS |
| | LCDR3 | 36 | HQRSTYPLT |
| Antibody to GITR | HCDR1 | 37 | SYGMH |
| | HCDR2 | 38 | VIWYEGSNKYYADSVKG |
| | HCDR3 | 39 | GGSMVRGDYYYGMDV |
| | LCDR1 | 40 | RASQGISSALA |
| | LCDR2 | 41 | DASSLES |
| | LCDR3 | 42 | QQFNSYPYT |
| Antibody to LAG-3 | HCDR1 | 43 | DYYWN |
| | HCDR2 | 44 | EINHRGSTNSNPSLKS |
| | HCDR3 | 45 | GYSDYEYNWFDP |
| | LCDR1 | 46 | RASQSISSYLA |
| | LCDR2 | 47 | DASNRAT |
| | LCDR3 | 48 | QQRSNWPLT |
| Utomilumab | HCDR1 | 50 | GYSFSTYW |
| | HCDR2 | 51 | IYPGDSYT |
| | HCDR3 | 52 | ARGYGIFDY |
| | LCDR1 | 53 | NIGDQY |
| | LCDR2 | 54 | QDK |
| | LCDR3 | 55 | ATYTGFGSLAV |
| Antibody to | HCDR1 | 56 | GYTFTDYN |
| TNFR2 variant 1 | HCDR2 | 57 | INPNYEST |
| | HCDR3 | 58 | RDKGWYFDV |
| | LCDR1 | 59 | SSVKN |
| | LCDR2 | 60 | YTS |
| | LCDR3 | 61 | QQFTSSPYT |
| Antibody to | HCDR1 | 62 | GFSLSTSGMG |
| TNFR2 variant 2 | HCDR2 | 63 | IWWDDDK |
| | HCDR3 | 64 | ARLTGTRYFDY |
| | LCDR1 | 65 | QDINKF |
| | LCDR2 | 66 | YTS |
| | LCDR3 | 67 | LQYGNLWT |
| Antibody to | HCDR1 | 68 | GYTFTDYS |
| TNFR2 variant 3 | HCDR2 | 69 | INTETGEP |
| | HCDR3 | 70 | ATYYGSSYVPDY |
| | LCDR1 | 71 | QNVGTA |
| | LCDR2 | 72 | WTS |
| | LCDR3 | 73 | QYSDYPYT |
| Antibody to | HCDR1 | 74 | GYTFTDY |
| TNFR2 variant 4 | HCDR2 | 75 | WVDPEYGS |
| | HCDR3 | 76 | ARDDGSYSPFDY |
| | LCDR1 (major) | 77 | QNINKY |
| | LCDR2 (major) | 78 | YTS |
| | LCDR3 (major) | 79 | LQYVNLLT |
| | LCDR1 (minor) | 80 | ENVVTY |
| | LCDR2 (minor) | 81 | GAS |
| | LCDR3 (minor) | 82 | QGYSYPYT |
| Antibody | HCDR1 | 199 | DYYIH |
| huAD208.4.1 to | HCDR2 | 200 | LVYPYIGGTNYNQKFKG |
| LRRC15 | HCDR3 | 201 | GDNKYDAMDY |
| | LCDR1 | 202 | RASQSVSTSSYSYMH |
| | LCDR2 | 203 | YASSLES |
| | LCDR3 | 204 | EQSWEIRT |
| Antibody | HCDR1 | 205 | NYWMH |
| huAD208.12.1 to | HCDR2 | 206 | MIHPNSGSTKHNEKFRG |
| LRRC15 | HCDR3 | 207 | SDFGNYRWYFDV |
| | LCDR1 | 208 | RASQSSSNNLH |
| | LCDR2 | 209 | YVSQSIS |
| | LCDR3 | 210 | QQSNSWPFT |
| Antibody | HCDR1 | 211 | DYYIH |
| huAD208.14.1 to | HCDR2 | 212 | LVYPYIGGSSYNQQFKG |
| LRRC15 | HCDR3 | 213 | GDNNYDAMDY |
| | LCDR1 | 214 | RASQSVSTSTYNYMH |
| | LCDR2 | 215 | YASNLES |
| | LCDR3 | 216 | HHTWEIRT |
| Antibody hu139.10 | HCDR1 | 217 | SYGVH |
| to LRRC15 | HCDR2 | 218 | VIWAGGSTNYNSALMS |
| | HCDR3 | 219 | HMITEDYYGMDY |
| | LCDR1 | 220 | KSSQSLLNSRTRKNYLA |
| | LCDR2 | 221 | WASTRES |
| | LCDR3 | 222 | KQSYNLPT |
| Antibody | HCDR1 | 223 | NYWLG |
| muAD210.40.9 to | HCDR2 | 224 | DIYPGGGNTYYNEKLKG |
| LRRC15 | HCDR3 | 225 | WGDKKGNYFAY |
| | LCDR1 | 226 | TASSSVYSSYLH |
| | LCDR2 | 227 | STSNLAS |
| | LCDR3 | 228 | HQYHRSPT |
| Antibody | HCDR1 | 229 | NFGMN |
| muAD209.9.1 to | HCDR2 | 230 | WINLYTGEPTFADDFKG |
| LRRC15 | HCDR3 | 231 | KGETYYRYDGFAY |
| | LCDR1 | 232 | RSSKSLLHSNGNTHLY |
| | LCDR2 | 233 | RMSNLAS |
| | LCDR3 | 234 | MQLLEYPYT |

TABLE 2
Antibody V_{H} sequence and V_{L} sequences

| | | SEQ ID | |
|---|---|---|---|
| ANTIBODY | REGION | NO: | SEQUENCE: |
| Ipilumumab | V_{H} | 83 | QVQLVESGGGVVQPGRSLRLSCAASGFTFSSYTMH |
| | | | WVRQAPGKGLEWVTFISYDGNNKYYADSVKGRFTI |
| | | | SRDNSKNTLYLQMNSLRAEDTAIYYCARTGWLGPF |
| | | | DYWGQGTLVTVSS |
| | V_{L} | 84 | EIVLTQSPGTLSLSPGERATLSCRASQSVGSSYLAW |
| | | | YQQKPGQAPRLLIYGAFSRATGIPDRFSGSGSGTDFT |
| | | | LTISRLEPEDFAVYYCQQYGSSPWTFGQGTKVEIK |
| Opdivo ® | V_{H} | 85 | QVQLVESGGGVVQPGRSLRLDCKASGITFSNSGMH |
| (nivolumab) | | | WVRQAPGKGLEWVAVIWYDGSKRYYADSVKGRF |
| | | | TISRDNSKNTLFLQMNSLRAEDTAVYYCATNDDYW |
| | | | GQGTLVTVSS |
| | V_{L} | 86 | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQSSNWPRTFGQGTKVEIK |
| Keytruda ® | V_{H} | 87 | QVQLVQSGVEVKKPGASVKVSCKASGYTFTNYYM |
| (pembrolizumab) | | | YWVRQAPGQGLEWMGGINPSNGGTNFNEKFKNRV |
| | | | TLTTDSSTTTAYMELKSLQFDD |
| | | | TAVYYCARRDYRFDMGFDYWGQGTTVTVSS |
| | V_{L} | 88 | EIVLTQSPATLSLSPGERATLSCRASKGVSTSGYSYL |
| | | | HWYQQKPGQAPRLLIYLASYLESGVPARFSGSGSGT |
| | | | DFTLTISSLEPEDFAVYYCQHSRDLPLTFGGGTKVEI |
| | | | K |
| Atezolizumab | V_{H} | 89 | EVQLVESGGGLVQPGGSLRLSCAASGFTFSDSWIH |
| | | | WVRQAPGKGLEWVAWISPYGGSTYYADSVKGRFT |
| | | | ISADTSKNTAYLQMNSLRAEDTAVYYCARRHWPG |
| | | | GFDYWGQGTLVTVSS |
| | V_{L} | 90 | DIQMTQSPSSLSASVGDRVTITCRASQDVSTAVAWY |
| | | | QQKPGKAPKLLIYSASFLYSGVPSRFSGSGSGTDFTL |
| | | | TISSLQPEDFATYYCQQYLYHPATFGQGTKVEIK |
| Durvalumab | V_{H} | 91 | EVQLVESGGGLVQPGGSLRLSCAASGFTFSRYWMS |
| | | | WVRQAPGKGLEWVANIKQDGSEKYYVDSVKGRFT |
| | | | ISRDNAKNSLYLQMNSLRAEDTAVYYCAREGGWF |
| | | | GELAFDYWGQGTLVTVSS |
| | V_{L} | 92 | EIVLTQSPGTLSLSPGERATLSCRASQRVSSSYLAWY |
| | | | QQKPGQAPRLLIYDASSRATGIPDRFSGSGSGTDFTL |
| | | | TISRLEPEDFAVYYCQQYGSLPWTFGQGTKVEIK |
| MDX-1106 | V_{H} | 93 | QVQLVQSGAEVKKPGSSVKVSCKTSGDTFSTYAIS |
| | | | WVRQAPGQGLEWMGGIIPIFGKAHYAQKFQGRVTI |
| | | | TADESTSTAYMELSSLRSEDTAVYFCARKFHFVSGS |
| | | | PFGMDVWGQGTTVTVSS |
| | V_{L} | 94 | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQRSNWPTFGQGTKVEIK |
| Antibody to | V_{H} | 95 | EVQLQQSGAELVKPGASVKISCKASGYTFTDYNMD |
| TNFR2 variant 1 | | | WVKQSHGKSLEWIGDINPNYESTSYNQKFKGKATL |
| | | | TVDKSSSTAYMEVRSLTSEDTAVFYCARDKGWYFD |
| | | | VWGAGTTVTVSS |
| | V_{L} | 96 | ENVLTQSPAIMSASLGEKVTMSCRASSSVKNMYWY |
| | | | QQKSDASPKLWIYYTSNLAPGVPARFSGSGSGNSYS |
| | | | LTISSMEGEDAATYYCQQFTSSPYTFGGGTKLELK |
| Antibody to | V_{H} | 97 | QVTLKESGPGILQPSQTLSLTCSFSGFSLSTSGMGVG |
| TNFR2 variant 2 | | | WIRQPSGKGLEWLAHIWWDDDKFYNPSLKSQLTIS |
| | | | KDTSRNQVFLKLTSVVTADTATYYCARLTGTRYFD |
| | | | YWGQGTTLTVSS |
| | V_{L} | 98 | DVQMTQSPSSLSASLGGKVTITCKASQDINKFIAWY |
| | | | QHKPGKGPRLLIHYTSTLQPGIPSKFSGSGSGRDYSF |
| | | | SISNLEPEDIATYYCLQYGNLWTFGGGTKLEIT |
| Antibody to | V_{H} | 99 | QIQLVQSGPELKKPGETVKISCKASGYTFTDYSMH |
| TNFR2 variant 3 | | | WVKQAPGKGLKWMGWINTETGEPTYADDFKGRF |
| | | | AFSSETSTSTAYLQINNLKNDDTTTYFCATYYGSSY |
| | | | VPDYWGQGTSLTVSS |
| | V_{L} | 100 | DIVMTQSHKFMSTSVGDRVSITCKASQNVGTAVAW |
| | | | YQHKPGQSPKLLIYWTSSRHTGVPDRFTGSGSGTEF |
| | | | TLTISNVQSEDLADYFCHQYSDYPYTFGGGTKLEIK |
| Antibody to | V_{H} | 101 | EVQLQQSGPEVGRPGSSVKISCKASGYTFTDYIMH |
| TNFR2 variant 4 | | | WVKQSPGQGLEWIGWVDPEYGSTDYAEKFKKKAT |
| | | | LTADTSSNTAYIQLSSLTSEDTATYFCARDDGSYSPF |
| | | | DYWGQGVMVTVSS |
| | V_{L} | 102 | DIQMTQSPPSLSASLGDKVTITCQASQNINKYIAWY |
| | (major) | | QQKPGKAPRLLIRYTSTLESGTPSRFSGSGSGRDYSF |
| | | | SISNVESEDIASYYCLQYVNLLTFGAGTKLEIK |
| | V_{L} | 103 | NIVMTQSPKSMSMSVGERVTLTCKASENVVTYVSW |
| | (minor) | | YQQKPEQSPKLLIYGASNRYTGVPDRFTGSGSATDF |
| | | | TLTISSVQAEDLADYHCGQGYSYPYTFGGGTKLEIK |
| Antibody to | V_{H} | 104 | EVQLVESGGGLVQPGGSLRLSCAASGFTFSDYAMS |
| TNFR2 variant 5 | | | WVRQAPGKGLEWVAVISENGSDTYYADSVKGRFTI |
| | | | SRDDSKNTLYLQMNSLRAEDTAVYYCARDRGGAV |
| | | | SYFDVWGQGTLVTVSS |
| | V_{L} | 105 | DIQMTQSPSSLSASVGDRVTITCRASQDVSSYLAWY |
| | | | QQKPGKAPKLLIYAASSLESGVPSRFSGSGSGTDFTL |
| | | | TISSLQPEDFATYYCQQYNSLPYTFGQGTKVEIKRT |
| Vonlerolizumab | V_{H} | 106 | EVQLVQSGAEVKKPGASVKVSCKASGYTFTDSYMS |
| | | | WVRQAPGQGLEWIGDMYPDNGDSSYNQKFRERVT |
| | | | ITRDTSTSTAYLELSSLRSEDTAVYYCVLAPRWYFS |
| | | | VWGQGTLVTVSS |
| | V_{L} | 107 | DIQMTQSPSSLSASVGDRVTITCRASQDISNYLNWY |
| | | | QQKPGKAPKLLIYYTSRLRSGVPSRFSGSGSGTDFTL |
| | | | TISSLQPEDFATYYCQQGHTLPPTFGQGTKVEIK |
| TRX518 | V_{L} | 108 | EIVMTQSPATLSVSPGERATLSCKASQNVGTNVAW |
| | | | YQQKPGQAPRLLIYSASYRYSGIPARFSGSGSGTEFT |
| | | | LTISSLQSEDFAVYYCQQYNTDPLTFGGGTKVEIK |
| | V_{H} | 109 | QVTLRESGPALVKPTQTLTLTCTFSGFSLSTSGMGV |
| | | | GWIRQPPGKALEWLAHIWWDDDKYYNPSLKSRLTI |
| | | | SKDTSKNQVVLTMTNMDPVDTATYYCARTRRYFP |
| | | | FAYWGQGTLVTVSS |
| | V_{H} | 110 | QVTLRESGPALVKPTQTLTLTCTFSGFSLSTSGMGV |
| | | | GWIRQPPGKALEWLAHIWWDDDKYYQPSLKSRLTI |
| | | | SKDTSKNQVVLTMTNMDPVDTATYYCARTRRYFP |
| | | | FAYWGQGTLVTVSS |
| Antibody to | V_{H} | 111 | QVQLQQWGAGLLKPSETLSLTCAVYGGSFSDYYW |
| LAG-3 | | | NWIRQPPGKGLEWIGEINHRGSTNSNPSLKSRVTLS |
| | | | LDTSKNQFSLKLRSVTAADTAVYYCAFGYSDYEYN |
| | | | WFDPWGQGTLVTVSS |
| | V_{L} | 112 | EIVLTQSPATLSLSPGERATLSCRASQSISSYLAWYQ |
| | | | QKPGQAPRLLIYDASNRATGIPARFSGSGSGTDFTLT |
| | | | ISSLEPEDFAVYYCQQRSNWPLTFGQGTNLEIK |
| Antibody to | V_{H} | 113 | MAVLALLFCLVTFPSCILSQVQLKESGPGLVAPSQS |
| GARP variant 1 | | | LSITCTVSGFSLTGYGINWVRQPPGKGLEWLGMIWS |
| | | | DGSTDYNSVLTSRLRISKDNSNSQVFLKMNSLQVD |
| | | | DTARYYCARDRNYYDYDGAMDYWGQGTSVTVSS |
| | V_{L} | 114 | QVQLKESGPGLVAPSQSLSITCTVSGFSLTGYGINW |
| | | | VRQPPGKGLEWLGMIWSDGSTDYNSVLTSRLRISK |
| | | | DNSNSQVFLKMNSLQVDDTARYYCARDRNYYDYD |
| | | | GAMDYWGQGTSVTVSS |
| Antibody to | V_{H} | 115 | MKFPSQLLLFLLFRITGIICDIQVTQSSSYLSVSLGDR |
| GARP variant 2 | | | VTITCKASDHIKNWLAWYQQKPGIAPRLLVSGATSL |
| | | | EAGVPSRFSGSGSGKNFTLSITSLQTEDVATYYCQQ |
| | | | YWSTPWTFGGGTTLEIR |
| | V_{L} | 116 | DIQVTQSSSYLSVSLGDRVTITCKASDHIKNWLAWY |
| | | | QQKPGIAPRLLVSGATSLEAGVPSRFSGSGSGKNFT |
| | | | LSITSLQTEDVATYYCQQYWSTPWTFGGGTTLEIR |
| Antibody to | V_{H} | 117 | EVQLVQPGAELRNSGASVKVSCKASGYRFTSYYID |
| GARP variant 3 | | | WVRQAPGQGLEWMGRIDPEDGGTKYAQKFQGRVT |
| | | | FTADTSTSTAYVELSSLRSEDTAVYYCARNEWETV |
| | | | VVGDLMYEYEYWGQGTQVTVSS |
| | V_{L} | 118 | DIQMTQSPTSLSASLGDRVTITCQASQSISSYLAWYQ |
| | | | QKPGQAPKLLIYGASRLQTGVPSRFSGSGSGTSFTLT |
| | | | ISGLEAEDAGTYYCQQYDSLPVTFGQGTKVELK |
| | V_{L} | 119 | DIQMTQSPSSLSASLGDRVTITCQASQSIVSYLAWY |
| | | | QQKPGQAPKLLIYGASRLQTGVPSRFSGSGSGTSFT |
| | | | LTISGLEAEDAGTYYCQQYASAPVTFGQGTGVELK |
| | V_{L} | 120 | DIQMTQSPSSLSASLGDRVTITCQASQSISSYLAWYQ |
| | | | QKPGQAPKLLIYGTSRLKTGVPSRFSGSGSGTSFTLT |
| | | | ISGLEAEDAGTYYCQQYYSAPVTFGQGTKVELK |
| | V_{L} | 121 | DIQMTQSPSSLSPSLGDRVTITCQASQTISSFLAWYH |
| | | | QKPGQPPKLLIYRASIPQTGVPSRFSGSGSGTSFTLTI |
| | | | GGLEAEDAGTYYCQQYVSAPPTFGQGTKVELK |
| | V_{L} | 122 | DIQMTQSPSSLSASLGDRVTITCQASQSISSYLAWYQ |
| | | | QKPGQAPNILIYGASRLKTGVPSRFSGSGSGTSFTLTI |
| | | | SGLEAEDAGTYYCQQYASVPVTFGQGTKVELK |
| Antibody to | V_{H} | 123 | QVQLQQWGAGLLKPSETLSLTCAVYGGSFSGYYW |
| 4-1BB variant 1 | | | SWIRQSPEKGLEWIGEINHGGYVTYNPSLESRVTISV |
| | | | DTSKNQFSLKLSSVTAADTAVYYCARDYGPGNYD |
| | | | WYFDLWGRGTLVTVSS |
| | V_{L} | 124 | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQRSNWPPALTFGGGTKVEI |
| | | | K |
| Antibody to | V_{H} | 125 | EVQLVESGGGLVQPGGSLRLSCAASGFTFSDYWMS |
| 4-1BB variant 2 | | | WVRQAPGKGLEWVADIKNDGSYTNYAPSLTNRFTI |
| | | | SRDNAKNSLYLQMNSLRAEDTAVYYCARELTGTW |
| | | | GQGTMVTVSS |
| | V_{L} | 126 | DIVMTQSPDSLAVSLGERATINCKSSQSLLSSGNQK |
| | | | NYLAWYQQKPGQPPKLLIYYASTRQSGVPDRFSGS |
| | | | GSGTDFTLTISSLQAEDVAVYYCLQYDRYPFTFGQG |
| | | | TKLEIK |
| Utomilumab | V_{H} | 127 | EVQLVQSGAEVKKPGESLRISCKGSGYSFSTYWISW |
| | | | VRQMPGKGLEWMGKIYPGDSYTNYSPSFQGQVTIS |
| | | | ADKSISTAYLQWSSLKASDTAMYYCARGYGIFDYW |
| | | | GQGTLVTVSS |
| | V_{L} | 128 | SYELTQPPSVSVSPGQTASITCSGDNIGDQYAHWYQ |
| | | | QKPGQSPVLVIYQDKNRPSGIPERFSGSNSGNTATLT |
| | | | ISGTQAMDEADYYCATYTGFGSLAVFGGGTKLTVL |
| Antibody to | V_{L} | 129 | DIQMTQSPSSVSASVGDRVTITCRASQGISRLLAWY |
| ICOS variant 1 | | | QQKPGKAPKLLIYVASSLQSGVPSRFSGSGSGTDFT |
| | | | LTISSLQPEDFATYYCQQANSFPWTFGQGTKVEIK |
| | V_{H} | 130 | QVQLVQSGAEVKKPGASVKVSCKASGYTFTGYYM |
| | | | HWVRQAPGQGLEWMGWINPHSGGTNYAQKFQGR |
| | | | VTMTRDTSISTAYMELSRLRSDDTAVYYCARTYYY |
| | | | DSSGYYHDAFDIWGQGTMVTVSS |
| Antibody to | V_{H} | 131 | EVQLVESGGGLVQPGGSLRLSCAASGFTFSDYWMD |
| ICOS variant 2 | | | WVRQAPGKGLVWVSNIDEDGSITEYSPFVKGRFTIS |
| | | | RDNAKNTLYLQMNSLRAEDTAVYYCTRWGRFGFD |
| | | | SWGQGTLVTVSS |
| | V_{L} | 132 | DIVMTQSPDSLAVSLGERATINCKSSQSLLSGSFNYL |
| | | | TWYQQKPGQPPKLLIFYASTRHTGVPDRFSGSGSGT |
| | | | DFTLTISSLQAEDVAVYYCHHHYNAPPTFGPGTKV |
| | | | DIK |
| Vorsetuzumab | V_{H} | 133 | QVQLVQSGAEVKKPGASVKVSCKASGYTFTNYGM |
| | | | NWVRQAPGQGLKWMGWINTYTGEPTYADAFKGR |
| | | | VTMTRDTSISTAYMELSRLRSDDTAVYYCARDYGD |
| | | | YGMDYWGQGTTVTVSS |
| | V_{L} | 134 | DIVMTQSPDSLAVSLGERATINCRASKSVSTSGYSF |
| | | | MHWYQQKPGQPPKLLIYLASNLESGVPDRFSGSGS |
| | | | GTDFTLTISSLQAEDVAVYYCQHSREVPWTFGQGT |
| | | | KVEIK |
| Rinucumab | V_{H} | 135 | QLQLQESGPGLVKPSETLSLTCTVSGGSITSSSYYW |
| | | | GWIRQPPGKGLEWIGSIYYRGSTNYNPSLKSRVTISV |
| | | | DSSKNQFYLKVSSVTAVDTAVYYCARQNGAARPS |
| | | | WFDPWGQGTLVTVSS |
| | V_{L} | 136 | EIVLTQSPDTISLSPGERATLSCRASQSISSIYLAWYQ |
| | | | QKPGQAPRLLIYGASSRVTGIPDRFSVSGSGTDFTLT |
| | | | ISRLEPEDFAVYYCQHYGISPFTFGPGTKVDIR |
| Oleclumab | V_{H} | 137 | EVQLLESGGGLVQPGGSLRLSCAASGFTFSSYAYSW |
| | | | VRQAPGKGLEWVSAISGSGGRTYYADSVKGRFTIS |
| | | | RDNSKNTLYLQMNSLRAEDTAVYYCARLGYGRVD |
| | | | EWGRGTLVTVSS |
| | V_{L} | 138 | QSVLTQPPSASGTPGQRVTISCSGSLSNIGRNPVNW |
| | | | YQQLPGTAPKLLIYLDNLRLSGVPDRFSGSKSGTSA |
| | | | SLAISGLQSEDEADYYCATWDDSHPGWTFGGGTKL |
| | | | TVL |
| Antibody to | V_{L} | 139 | DIQMTQSPSSLSASVGDRVTITCRASQGISSWLAWY |
| CD73 | | | QQKPEKAPKSLIYAASSLQSGVPSRFSGSGSGTDFTL |
| | | | TISSLQPEDFATYYCQQYNSYPLTFGGGTKVEIK |
| | V_{H} | 140 | QVQLVESGGGVVQPGRSLRLSCAASGFTFSNYGMH |
| | | | WVRQAPGKGLEWVAVILYDGSNKYYPDSVKGRFTI |
| | | | SRDNSKNTLYLQMNSLRAEDTAVYYCARGGSSWY |
| | | | PDSFDIWGQGTMVTVSS |
| Daratumumab | V_{H} | 141 | EVQLLESGGGLVQPGGSLRLSCAVSGFTFNSFAMS |
| | | | WVRQAPGKGLEWVSAISGSGGGTYYADSVKGRFTI |
| | | | SRDNSKNTLYLQMNSLRAEDTAVYFCAKDKILWFG |
| | | | EPVFDYWGQGTLVTVSS |
| | V_{L} | 142 | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQRSNWPPTFGQGTKVEIK |
| Etaracizumab | V_{H} | 143 | QVQLVESGGGVVQPGRSLRLSCAASGFTFSSYDMS |
| | | | WVRQAPGKGLEWVAKVSSGGGSTYYLDTVQGRFT |
| | | | ISRDNSKNTLYLQMNSLRAEDTAVYYCARHLHGSF |
| | | | ASWGQGTTVTVSS |
| | V_{L} | 144 | EIVLTQSPATLSLSPGERATLSCQASQSISNFLHWYQ |
| | | | QRPGQAPRLLIRYRSQSISGIPARFSGSGSGTDFTLTI |
| | | | SSLEPEDFAVYYCQQSGSWPLTFGGGTKVEIK |
| Intetumumab | V_{H} | 145 | QVQLVESGGGVVQPGRSRRLSCAASGFTFSRYTMH |
| | | | WVRQAPGKGLEWVAVISFDGSNKYYVDSVKGRFTI |
| | | | SRDNSENTLYLQVNILRAEDTAVYYCAREARGSYA |
| | | | FDIWGQGTMVTVSS |
| | V_{L} | 146 | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQRSNWPPFTFGPGTKVDIK |
| Antibody to | V_{H} | 147 | EVQLVESGGGLVQPGGSLRLSCAVSGFVFSRYWMS |
| Integrin avβ8 | | | WVRQAPGKGLEWIGEINPDSSTINYTSSLKDRFTISR |
| | | | DNAKNSLYLQMNSLRAEDTAVYYCASLITTEDYW |
| | | | GQGTTVTVSS |
| | V_{L} | 148 | EIVLTQSPSSLSLSPGERVTITCKASQDINSYLSWYQ |
| | | | QKPGKAPKLLIYYANRLVDGVPARFSGSGSGQDYT |
| | | | LTISSLEPEDFAVYYCLQYDEFPYTFGGGTKLEIKR |
| Ontuxizumab | V_{H} | 149 | QVQLQESGPGLVRPSQTLSLTCTASGYTFTDYVIHW |
| | | | VKQPPGRGLEWIGYINPYDDDTTYNQKFKGRVTML |
| | | | VDTSSNTAYLRLSSVTAEDTAVYYCARRGNSYDGY |
| | | | FDYSMDYWGSGTPVTVSS |
| | V_{L} | 150 | DIQMTQSPSSLSASVGDRVTITCRASQNVGTAVAW |
| | | | LQQTPGKAPKLLIYSASNRYTGVPSRFSGSGSGTDY |
| | | | TFTISSLQPEDIATYYCQQYTNYPMYTFGQGTKVQI |
| | | | K |
| Antibody to | V_{H} | 151 | QVQLQESGPGLVKPSQTLSLTCAISGDSVSSNSVTW |
| FAP variant 1 | | | NWIRQSPSRGLEWLGRTYYRSKWYNDYAVSVKGR |
| | | | ITINPDTSKNQFYLQLKSVTPEDAAVYYCARDSSILY |
| | | | GDYWGQGTLVTVSS |
| | V_{H} | 152 | QVQLQQSGPGLVKPSQTLSLTCAISGDSVSSNSVTW |
| | | | NWIRQSPSRGLEWLGRTYYRSKWYNDYAVSVKGR |
| | | | ITINPDTSKNQFYLQLKSVTPEDAAVYYCARDSSILY |
| | | | GDYWGQGTLVTVS |
| | V_{L} | 153 | QAVLTQPSSLSASPGASASLTCTLPSGINVGTYRIFW |
| | | | FQQKPGSPPQYLLSYKSDSDNHQGSGVPSRFSGSKD |
| | | | ASANAGILLISGLQSEDEADYYCMIWHSSAWVFGG |
| | | | GTKLTVL |
| Antibody to | V_{H} | 154 | QVQLVQSGAEVKKPGASVKVSCKTSGYTFTDYYIH |
| FAP variant 2 | | | WVRQAPGQGLEWMGWINPNRGGTNYAQKFQGRV |
| | | | TMTRDTSIATAYMELSRLRSDDTAVYYCATASLKIA |
| | | | AVGTFDCWGQGTLVTVSS |
| | V_{L} | 155 | SYELTQPPSVSVSPGQTARITCSGDALSKQYAFWFQ |
| | | | QKPGQAPILVIYQDTKRPSGIPGRFSGSSSGTTVTLTI |
| | | | SGAQADDEADYYCQSADSSGTYVFGTGTKVTVL |
| Antibody to | V_{H} | 156 | EVQLVETGGGVVQPGRSLRLSCAASGFSFSTHGMY |
| FAP variant 3 | | | WVRQPPGKGLEWVAVISYDGSDKKYADSVKGRFTI |
| | | | SRDNSKNTVYLEMSSVRAEDTALYYCFCRRDAFDL |
| | | | WGQGTMVTVSS |
| | V_{L} | 157 | SYVLTQPPSVSVSPGQTARITCSGDALPKKYAYWY |
| | | | QQKSGQAPVLVIYEDTKRPSGIPERFSGSSSGTMATL |
| | | | TISGAQVEDEADYYCYSTDSSGNYWVFGGGTEVTV |
| | | | L |
| Antibody to | V_{H} | 158 | EVQLVESGGGLVEPGGSLRLSCAASGFTFSDAWMN |
| FAP variant 4 | | | WVRQAPGKGLEWVGRIKTKSDGGTTDYAAPVRGR |
| | | | FSISRDDSKNTLFLEMNSLKTEDTAIYYCFITVIVVSS |
| | | | ESPLDHWGQGTLVTVSS |
| | V_{L} | 159 | SYELTQPPSVSVSPGQTARITCSGDELPKQYAYWYQ |
| | | | QKPGQAPVLVIYKDRQRPSGIPERFSGSSSGTTVTLT |
| | | | ISGVQAEDEADYYCQSAYSINTYVIFGGGTKLTVL |
| Antibody to | V_{H} | 160 | EVQLVESGGGLVKPGGSLRLSCAASGFTFSDYYMS |
| FAP variant 5 | | | WIRQAPGKGLEWISYISSGSSYTNYADSVKGRFTISR |
| | | | DNAKKSVYLEVNGLTVEDTAVYYCARVRYGDREM |
| | | | ATIGGFDFWGQGTLVTVSS |
| | V_{L} | 161 | SYELTQPPSVSVSPGQTARITCSGDALPKQYAYWYQ |
| | | | QSPGQAPVLVIYKDSERPSGIPERFSGSSSGTTVTLTI |
| | | | SGVQAEDEADYYCQSADSGGTSRIFGGGTKLTVL |
| Antibody to | V_{H} | 162 | QVQLQESGPGLVRSTETLSLTCLVSGDSINSHYWSW |
| FAP variant 6 | | | LRQSPGRGLEWIGYIYYTGPTNYNPSLKSRVSISLGT |
| | | | SKDQFSLKLSSVTAADTARYYCARNKVFWRGSDFY |
| | | | YYMDVWGKGTTVTVSS |
| | V_{L} | 163 | EIVLTQSPGTLSLSLGERATLSCRASQSLANNYLAW |
| | | | YQQKPGQAPRLLMYDASTRATGIPDRFSGSGSGTDF |
| | | | TLTISRLEPEDFAVYYCQQFVTSHHMYIFGQGTKVE |
| | | | IK |
| Antibody to | V_{H} | 164 | HVQLQESGPGLVKPSETLSLTCTVSGGSISSNNYYW |
| FAP variant 7 | | | GWIRQTPGKGLEWIGSIYYSGSTNYNPSLKSRVTISV |
| | | | DTSKNQFSLKLSSVTAADTAVYYCARGARWQARP |
| | | | ATRIDGVAFDIWGQGTMVTVSS |
| | V_{H} | 165 | QVQLQESGPGLVKPSETLSLTCTVSGGSISSNNYYW |
| | | | GWIRQTPGKGLEWIGSIYYSGSTNYNPSLKSRVTISV |
| | | | DTSKNQFSLKLSSVTAADTAVYYCARGARWQARP |
| | | | ATRIDGVAFDIWGQGTMVTVSS |
| | V_{H} | 166 | EVQLVQSGAEVKKPGASVKVSCKASGYTFTSYGIS |
| | | | WVRQAPGQGLEWMGWISAYNGNTNYAQKLQGRV |
| | | | TMTTDTSTSTAYMELRSLRSDDTAVYYCARDWSRS |
| | | | GYYLPDYWGQGTLVTVSS |
| | V_{L} | 167 | ETTLTQSPGTLSLSPGERATLSCRASQTVTRNYLAW |
| | | | YQQKPGQAPRLLMYGASNRAAGVPDRFSGSGSGT |
| | | | DFTLTISRLEPEDFAVYYCQQFGSPYTFGQGTKVEIK |
| | V_{L} | 168 | DVVMTQSPLSLPVTLGQPASISCRSSQSLLHSNGYN |
| | | | YLDWYLQRPGQSPHLLIFLGSNRASGVPDRFSGSGS |
| | | | GTDFTLKISRVEAEDVGIYYCMQALQTPPTFGQGTK |
| | | | VEIK |
| Antibody to | V_{H} | 171 | QVQLVESGGGVVQPGRSRRLSCAASGFTFSRYTMH |
| Integrin αv | | | WVRQAPGKGLEWVAVISFDGSNKYYVDSVKGRFTI |
| variant 1 | | | SRDNSENTLYLQVNILRAEDTAVYYCAREARGSYA |
| | | | FDIWGQGTMVTVSS |
| | V_{L} | 172 | EIVLTQSPATLSLSPGERATLSCRASQSVSSYLAWY |
| | | | QQKPGQAPRLLIYDASNRATGIPARFSGSGSGTDFT |
| | | | LTISSLEPEDFAVYYCQQRSNWPPFTFGPGTKVDIK |
| Antibody to | V_{L} | 173 | DIQMTQSPSSLSASVGDRVTITCRASQDISNYLAWY |
| Integrin αv | | | QQKPGKAPKLLIYYTSKIHSGVPSRFSGSGSGTDYTF |
| variant 2 | | | TISSLQPEDIATYYCQQGNTFPYTFGQGTKVEIK |
| | V_{H} | 174 | QVQLQQSGGELAKPGASVKVSCKASGYTFSSFWM |
| | | | HWVRQAPGQGLEWIGYINPRSGYTEYNEIFRDKAT |
| | | | MTTDTSTSTAYMELSSLRSEDTAVYYCASFLGRGA |
| | | | MDYWGQGTTVTVSS |
| Antibody to | V_{H} | 175 | QVQLQESGPGLVKPSQTLSLTCTVSGGSISSGVYYW |
| Integrin αvβ6 | | | TWIRQHPGNGLEWIGYIYYSGSTSYNPSLKSRVTISV |
| variant 1 | | | DTSKKQFSLNLTSVTAADTAVYYCAREGPLRGDYY |
| | | | YGLDVWGQGTTVTVSS |
| | V_{L} | 176 | EIVLTQSPGTLSLSPGERATLSCRAGQTISSRYLAWY |
| | | | QQKPGQAPRPLIYGASSRATGIPDRFSGSGSGTDFTL |
| | | | TISRLEPEDFAVYYCQQYGSSPRTFGQGTKVEIK |
| Antibody to | V_{H} | 177 | QVQLQESGPGLVKPSQTLSLTCTVSGGSISSGGYYW |
| Integrin αvβ6 | | | SWIRQHPGKGLEWIGYIYYSGSTYYNPSLKSRVTIS |
| variant 2 | | | VDTSKNQFSLKLSSVTAADTAMYYCARYRGPAAG |
| | | | RGDFYYFGMDVWGQGTTVTVSS |
| | V_{L} | 178 | DIVMTQTPLSLSVTPGQPASIFCKSSQSLLNSDGKTY |
| | | | LCWYLQKPGQPPQLLIYEVSNRFSGVPDRFSGSGSG |
| | | | TDFTLKISRVEAEDVGVYYCMQGIQLPWAFFGQGT |
| | | | KVEIK |
| Antibody to | V_{H} | 179 | QVQLVESGGGVVQPGRSLRLSCAASGFTFSSYGMH |
| Integrin αvβ6 | | | WVRQAPGKGLEWVAVIWYGGSNKYYADSVKGRF |
| variant 3 | | | TISRDNSKNTLYLQMNSLRAEDTAVYYCARDLAAR |
| | | | RGDYYYYGMDVWGQGTTVTVSS |
| | V_{L} | 180 | SSELTQDPVVSVALGQTVRITCQGDSLRSYYLSWY |
| | | | QQKPGQAPVLVIYGKNNRPSGIPDRFSGSNSGNTAS |
| | | | LTITGAQAEDEADYYCNSRDSSGNHLFGGGTKLTV |
| | | | L |
| Antibody to | V_{H} | 181 | QVQLQESGPGLVKPSQTLSLTCTVSGGSISSGGYYW |
| Integrin αvβ6 | | | SWIRQHPGKGLEWIGYIYYSGRTYNNPSLKSRVTIS |
| variant 4 | | | VDTSKNQFSLKLSSVTAADTAVYYCARVATGRAD |
| | | | YHFYAMDVWGQGTTVTVSS |
| | V_{L} | 182 | SYELTQPSSVSVSPGQTARITCSGDVLAKKSARWFH |
| | | | QKPGQAPVLVIYKDSERPSGIPERFSGSSSGTTVTLTI |
| | | | SGAQVEDEAAYYCYSAADNNLVFGGGTKLTVL |
| Varlilumab | V_{H} | 183 | QVQLVESGGGVVQPGRSLRLSCAASGFTFSSYDMH |
| | | | WVRQAPGKGLEWVAVIWYDGSNKYYADSVKGRF |
| | | | TISRDNSKNTLYLQMNSLRAEDTAVYYCARGSGN |
| | | | WGFFDYWGQGTLVTVSS |
| | V_{L} | 184 | DIQMTQSPSSLSASVGDRVTITCRASQGISRWLAWY |
| | | | QQKPEKAPKSLIYAASSLQSGVPSRFSGSGSGTDFTL |
| | | | TISSLQPEDFATYYCQQYNTYPRTFGQGTKVEIK |
| Zinbryta ® | V_{H} | 185 | QVQLVQSGAEVKKPGSSVKVSCKASGYTFTSYRMH |
| (Daclizumab) | | | WVRQAPGQGLEWIGYINPSTGYTEYNQKFKDKATI |
| | | | TADESTNTAYMELSSLRSEDTAVYYCARGGGVFDY |
| | | | WGQGTLVTVSS |
| | V_{L} | 186 | DIQMTQSPSTLSASVGDRVTITCSASSSISYMHWYQ |
| | | | QKPGKAPKLLIYTTSNLASGVPARFSGSGSGTEFTLT |
| | | | ISSLQPDDFATYYCHQRSTYPLTFGQGTKVEVK |
| Antibody to | V_{H} | 187 | QVQLVESGGGVVQPGRSLRLSCAASGFTFSSYGMH |
| GITR | | | WVRQAPGKGLEWVAVIWYEGSNKYYADSVKGRF |
| | | | TISRDNSKNTLYLQMNSLRAEDTAVYYCARGGSMV |
| | | | RGDYYYGMDVWGQGTTVTVSS |
| | V_{L} | 188 | AIQLTQSPSSLSASVGDRVTITCRASQGISSALAWYQ |
| | | | QKPGKAPKLLIYDASSLESGVPSRFSGSGSGTDFTLT |
| | | | ISSLQPEDFATYYCQQFNSYPYTFGQGTKLEIK |
| Antibody | V_{H} | 189 | EVQLVQSGAEVKKPGASVKVSCKASGYKFSSYWIE |
| huM25 to | | | WVKQAPGQGLEWIGEILPGSDTTNYNEKFKDRATF |
| LRRC15 | | | TSDTSINTAYMELSRLRSDDTAVYYCARDRGNYRA |
| | | | WFGYWGQGTLVTVSS |
| | V_{L} | 190 | DIQMTQSPSSLSASVGDRVTITCRASQDISNYLNWY |
| | | | QQKPGGAVKFLIYYTSRLHSGVPSRFSGSGSGTDYT |
| | | | LTISSLQPEDFATYFCQQGEALPWTFGGGTKVEIK |
| Antibody | V_{H} | 191 | EVQLVQSGAEVKKPGSSVKVSCKASGFTFTDYYIH |
| huAD208.4.1 to | | | WVKQAPGQGLEWIGLVYPYIGGTNYNQKFKGKAT |
| LRRC15 | | | LTVDTSTTTAYMEMSSLRSEDTAVYYCARGDNKY |
| | | | DAMDYWGQGTTVTVSS |
| | V_{L} | 192 | DIVLTQSPDSLAVSLGERATINCRASQSVSTSSYSYM |
| | | | HWYQQKPGQPPKLLIKYASSLESGVPDRFSGSGSGT |
| | | | DFTLTISSLQ |
| | | | AEDVAVYYCEQSWEIRTFGGGTKVEIK |
| Antibody | V_{H} | 193 | EVQLVQSGAEVKKPGSSVKVSCKASGYTFTNYWM |
| huAD208.12.1 | | | HWVKQAPGQGLEWIGMIHPNSGSTKHNEKFRGKA |
| to LRRC15 | | | TLTVDESTTTAYMELSSLRSEDTAVYYCARSDFGN |
| | | | YRWYFDVWGQGTTVTVSS |
| | V_{L} | 194 | EIVLTQSPATLSLSPGERATLSCRASQSSSNNLHWY |
| | | | QQKPGQAPRVLIKYVSQSISGIPARFSGSGSGTDFTL |
| | | | TISSLEPEDFA VYFCQQSNSWPFTFGQGTKLEIK |
| Antibody | V_{H} | 195 | EVQLVQSGAEVKKPGSSVKVSCKASGFTFTDYYIH |
| huAD208.14.1 | | | WVKQAPGQGLEWIGLVYPYIGGSSYNQQFKGKATL |
| to LRRC15 | | | TVDTSTSTAYMELSSLRSEDTAVYYCARGDNNYDA |
| | | | MDYWGQGTTVTVSS |
| | V_{L} | 196 | DIVLTQSPDSLAVSLGERATISCRASQSVSTSTYNYM |
| | | | HWYQQKPGQPPKLLVKYASNLESGVPDRFSGSGSG |
| | | | TDFTLTISSL |
| | | | QAEDVAVYYCHHTWEIRTFGGGTKVEIK |
| Antibody | V_{H} | 197 | EVQLVESGGGLVQPGGSLRLSCAVSGFSLTSYGVH |
| hu139.10 to | | | WVRQATGKGLEWLGVIWAGGSTNYNSALMSRLTI |
| LRRC15 | | | SKENAKSSVYLQMNSLRAGDTAMYYCATHMITED |
| | | | YYGMDYWGQGTTVTVSS |
| | V_{L} | 198 | DIVMTQSPDSLAVSLGERATINCKSSQSLLNSRTRK |
| | | | NYLAWYQQKPGQSPKLLIYWASTRESGVPDRFSGS |
| | | | GSGTDFTLTISS |
| | | | LQAEDVAVYYCKQSYNLPTFGGGTKVEIK |

### Target Binding Domain

An antibody construct may further comprise a target binding domain. A target binding domain may comprise a domain that binds to a target. A target may be an antigen. A target binding domain may comprise an antigen binding domain. A target binding domain may be a domain that can specifically bind to an antigen. A target binding domain may be an antigen-binding portion of an antibody or an antibody fragment. A target binding domain may be one or more fragments of an antibody that can retain the ability to specifically bind to an antigen. A target binding domain may be any antigen binding fragment. A target binding domain may be in a scaffold, in which a scaffold is a supporting framework for the antigen binding domain. A target binding domain may comprise an antigen binding domain in a scaffold.

A target binding domain may comprise an antigen binding domain which refers to a portion of an antibody comprising the antigen recognition portion, i.e., an antigenic determining variable region of an antibody sufficient to confer recognition and binding of the antigen recognition portion to a target, such as an antigen, i.e., the epitope. A target binding domain may comprise an antigen binding domain of an antibody.

An Fv can be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) can recognize and bind antigen, although at a lower affinity than the entire binding site.

A target binding domain may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

A target binding domain may be attached to an antibody construct. For example, an antibody construct may be fused with a target binding domain to create an antibody construct target binding domain fusion. The antibody construct-target binding domain fusion may be the result of the nucleic acid sequence of the target binding domain being expressed in frame with the nucleic acid sequence of the antibody construct. The antibody construct-target binding domain fusion may be the result of an in-frame genetic nucleotide sequence or a contiguous peptide sequence encoding the antibody construct with the target binding domain. As another example, a target binding domain may be linked to an antibody construct. A target binding domain may be linked to an antibody construct by a chemical conjugation. A target binding domain may be attached to a terminus of an Fc region. A target binding domain may be attached to a terminus of an Fc domain. A target binding domain may be attached to a terminus of an antibody construct. A target binding domain may be attached to a terminus of an antibody. A target binding domain may be attached to a light chain of an antibody. A target binding domain may be attached to a terminus of a light chain of an antibody. A target binding domain may be attached to a heavy chain of an antibody. A target binding domain may be attached to a terminus of a heavy chain of an antibody. The terminus may be a C-terminus. An antibody construct may be attached to 1, 2, 3, and/or 4 target binding domains. The target binding domain may direct the antibody construct to, for example, a particular cell or cell type. A target binding domain of an antibody construct may be selected in order to recognize an antigen, e.g., an antigen expressed on an immune cell. An antigen can be a peptide or fragment thereof. An antigen may be expressed on an antigen-presenting cell. An antigen may be expressed on a dendritic cell, a macrophage, or a B cell. As another example, an antigen may be a tumor antigen. The tumor antigen may be any tumor antigen described herein. When multiple target binding domains are attached to an antibody construct, the target binding domains may bind to the same antigen. When multiple target binding domains are attached to an antibody construct, the target binding domains may bind different antigens.

In certain embodiments, an antibody construct specifically binds a second antigen. In certain embodiments, the target binding domain is linked to said antibody construct at a C-terminal end of said Fc domain.

In certain embodiments, the target binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the target binding domain specifically binds to an antigen that is at least 80% identical to an antigen on a T cell, an APC, and/or a B cell. In certain embodiments, the target binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1. In certain embodiments, the target binding domain specifically binds to an antigen that is an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the target binding domain specifically binds to an antigen that is an antigen on a T cell, an APC, and/or a B cell. In certain embodiments, the target binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1.

### Attachment of Linkers to Antibody Construct

The conjugates described herein may comprise a linker, e.g., a peptide linker. Linkers of the conjugates and methods may not affect the binding of active portions of a conjugate (e.g., active portions include antigen binding domains, Fc domains, target binding domains, antibodies, amino-pyrazinecarboxamide compounds, inhibitors or the like) to a target, which can be a cognate binding partner such as an antigen. A linker can form a linkage between different parts of a conjugate, e.g., between an antibody construct or targeting moiety and a compound of the disclosure. In certain embodiments, a conjugate comprises multiple linkers. In certain embodiments, wherein a conjugate comprises multiple linkers, the linkers may be the same linkers or different linkers.

A linker may be bound to an antibody construct or targeting moiety by a bond between the antibody construct targeting moiety and the linker. A linker may be bound to an anti-tumor antigen antibody construct by a bond between the anti-tumor antigen antibody construct and the linker. A linker may be bound to a terminus of an amino acid sequence of an antibody construct, or could be bound to a side chain modification to the antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc region of an antibody construct, or may be bound to a side chain modification of an Fc region of an antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain of an antibody construct, or may be bound to a side chain modification of an Fc domain of an antibody construct, such as the side chain of a lysine, serine, threonine, cysteine, tyrosine, aspartic acid, glutamine, a non-natural amino acid residue, or glutamic acid residue.

A linker may be bound to an antibody construct at a hinge cysteine. A linker may be bound to an antibody construct at a light chain constant domain lysine. A linker may be bound to an antibody construct at an engineered cysteine in the light chain. A linker may be bound to an antibody construct at an Fc region lysine. A linker may be bound to an antibody construct at an Fc domain lysine. A linker may be bound to an antibody construct at an Fc region cysteine. A linker may be bound to an antibody construct at an Fc domain cysteine. A linker may be bound to an antibody construct at a light chain glutamine, such as an engineered glutamine. A linker may be bound to an antibody construct at an unnatural amino acid engineered into the light chain. A linker may be bound to an antibody construct at an unnatural amino acid engineered into the heavy chain. Amino acids can be engineered into an amino acid sequence of an antibody construct, for example, a linker of a conjugate. Engineered amino acids may be added to a sequence of existing amino acids. Engineered amino acids may be substituted for one or more existing amino acids of a sequence of amino acids.

A linker may be conjugated to an antibody construct via a sulfhydryl group on the antibody construct. A linker may be conjugated to an antibody construct via a primary amine on the antibody construct. A linker may be conjugated to an antibody construct via residue of an unnatural amino acid on an antibody construct, e.g., a ketone moiety.

In certain embodiments, when one or more linkers are bound, e.g., covalently, to an antibody construct at sites on the construct, an Fc domain of the antibody construct can bind to Fc receptors. In certain embodiments, an antibody construct bound to a linker or an antibody construct bound to a linker bound to an amino-pyrazinecarboxamide compound, retains the ability of the Fc domain of the antibody to bind to Fc receptors. In certain embodiments, when a linker is connected to an antibody construct, the antigen binding domain of an antibody construct bound to a linker or an antibody construct bound to a linker bound to an amino-pyrazinecarboxamide compound can bind its antigen. In certain embodiments, when a linker is connected to an antibody construct at the sites described herein, a target binding domain of an antibody construct bound to a linker or an antibody construct bound to a linker bound to an amino-pyrazinecarboxamide compound can bind its antigen.

In certain embodiments, a linker or linker bound to an amino-pyrazinecarboxamide compound may be attached to an amino acid residue of an IgG Fc domain selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335, 336, 396, 428, or any subset thereof, wherein numbering of amino acid residues in the Fc domain is according to the EU index as in Kabat.

In certain embodiments, a linker or linker bound to an amino-pyrazinecarboxamide compound is not attached to an amino acid residue of an IgG Fc domain selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335, 336, 396, 428, or any subset thereof, wherein numbering of amino acid residues in the Fc domain is according to the EU index as in Kabat.

### Lysine-Based Bioconjugation

An antibody construct can be conjugated to a linker via lysine-based bioconjugation. An antibody construct can be exchanged into an appropriate buffer, for example, phosphate, borate, PBS, histidine, Tris-Acetate at a concentration of about 2 mg/mL to about 10 mg/mL. An appropriate number of equivalents of a construct of an amino-pyrazinecarboxamide compound, and a linker, linker-payload, as described herein, can be added as a solution with stirring. Dependent on the physical properties of the linker-payload, a co-solvent can be introduced prior to the addition of the linker-payload to facilitate solubility. The reaction can be stirred at room temperature for 2 hours to about 12 hours depending on the observed reactivity. The progression of the reaction can be monitored by LC-MS. Once the reaction is deemed complete, the remaining linker-payloads can be removed by applicable methods and the antibody conjugate can be exchanged into the desired formulation buffer. Lysine-linked conjugates can be synthesized starting with ab antibody (mAb) and linker-payload, e.g., 10 equivalents, following Scheme A below (Conjugate=antibody conjugate). Monomer content and drug-antibody construct ratios (molar ratios) can be determined by methods described herein.

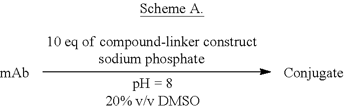

### Cysteine-Based Bioconjugation

An antibody construct can be conjugated to a linker via cysteine-based bioconjugation. An antibody construct can be exchanged into an appropriate buffer, for example, phosphate, borate, PBS, histidine, Tris-Acetate at a concentration of about 2 mg/mL to about 10 mg/mL with an appropriate number of equivalents of a reducing agent, for example, dithiothreitol or tris(2-carboxyethyl)phosphine. The resultant solution can be stirred for an appropriate amount of time and temperature to effect the desired reduction. A construct of an amino-pyrazinecarboxamide compound and a linker can be added as a solution with stirring. Dependent on the physical properties of the linker-payload, a co-solvent can be introduced prior to the addition of the linker-payload to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free linker-payload can be removed by applicable methods and the antibody conjugate can be exchanged into the desired formulation buffer. Such cysteine-based conjugates can be synthesized starting with an antibody (mAb) and linker-payload, e.g., 7 equivalents, using the conditions described in Scheme B below (Conjugate=antibody conjugate). Monomer content and drug-antibody ratios can be determined by methods described herein.

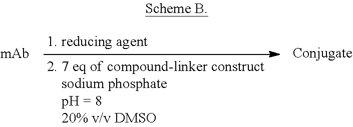

### Compounds

The following is a discussion of compounds and salts thereof that may be used in the methods of the disclosure. The compounds and salts described in Formulas (I-A), (I-B), (I-C), (I-D), (I-E) (II-A), (II-B), (II-C), and (II-D) and Table 14 may be covalently bound, to linkers, L³, which may further be covalently bound to antibody constructs or targeting moieties.

In a first aspect, disclosed herein is a compound represented by Formula (I):

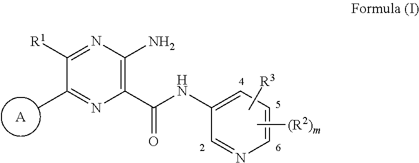

or a pharmaceutically acceptable salt thereof, wherein:
Ring A is unsubstituted or substituted cycloalkyl, unsubstituted or substituted heterocycloalkyl, unsubstituted or substituted aryl, or unsubstituted or substituted heteroaryl, wherein when Ring A is substituted, substituents on Ring A are independently selected at each occurrence from R⁴;
   each R⁴ is selected from R^{L} and R²⁰, or two R⁴ on adjacent atoms are taken together with the atoms to which they are attached to form an unsubstituted or substituted monocyclic carbocycle or unsubstituted or substituted monocyclic heterocycle;
   R^{L} is

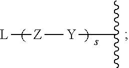

each Y is independently unsubstituted or substituted C₁-C₆alkylene; wherein when Y is substituted, substituents on Y are independently selected at each occurrence from R⁵;
each R⁵ is selected from R²⁰, or two R⁵ on adjacent atoms are taken together with the atoms to which they are attached to form an unsubstituted or substituted monocyclic carbocycle, or unsubstituted or substituted monocyclic heterocycle;
each Z is independently —NR⁶S(═O)₂—, —S(═O)₂NR⁶—, —OC(═O)—, —C(═O)O—, —C(═O)NR⁶—, or —NR⁶C(═O)—; wherein each R⁶ is independently selected from hydrogen, unsubstituted or substituted C₁-C₆alkyl, unsubstituted or substituted carbocycle, and unsubstituted or substituted heterocycle, or an R⁵ and an R⁶ on adjacent atoms are taken together with the atoms to which they are attached to form an unsubstituted or substituted monocyclic heterocycle;
L is unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; wherein when L is substituted, substituents on L are independently selected at each occurrence from R⁷;
   each R⁷ is selected from —SSR⁵⁰ and R²⁰;
s is 1-10;
R¹ is selected from hydrogen and R²⁰;
each R² is independently selected from R²⁰, or two R² on adjacent atoms are taken together with the atoms to which they are attached to form an unsubstituted or substituted monocyclic carbocycle or unsubstituted or substituted monocyclic heterocycle;
m is 0-3;
R³ is selected from (i), (ii), (iii), and (iv):
   (i) unsubstituted or substituted aryl, or unsubstituted or substituted heteroaryl; wherein when R³ is substituted, substituents on R³ are independently selected at each occurrence from R¹⁰;
   (ii) unsubstituted or substituted cycloalkyl, or unsubstituted or substituted heterocycloalkyl; wherein when R³ is substituted, substituents on R³ are independently selected at each occurrence from R¹¹;
   (iii) unsubstituted or substituted polycyclic heterocycloalkyl, unsubstituted or substituted 3- to 5-membered monocyclic heterocycloalkyl, unsubstituted or substituted 6- to 8-membered monocyclic heterocycloalkyl comprising 1 or 2 N atoms and 1 or 2 other heteroatoms selected from O or S; wherein when R³ is substituted, substituents on R³ are independently selected at each occurrence from R¹¹; and

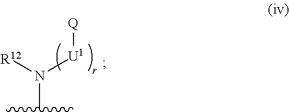

wherein when R³ is at the 2-, 5-, or 6-position of the pyridine, R³ is selected from (i), (ii), and (iv), and when R³ is at the 4-position of the pyridine, R³ is selected from (i), (iii), and (iv); and
   each R¹⁰ is selected from R²⁰, or two R¹⁰ on adjacent atoms are taken together with the atoms to which they are attached to form an unsubstituted or substituted monocyclic carbocycle or unsubstituted or substituted monocyclic heterocycle;
   each R¹¹ is selected from ═O, ═S, and R²⁰;
   R¹² is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   Q is —OR¹³, —NR¹³R¹³, —SR¹³, —CN, —C(═O)R¹⁴, —C(═O)NR¹³R¹³, —S(═O)R¹⁴, or —S(═O)₂R¹⁴, or —S(═O)₂NR¹³R¹³;
      R¹³ is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
      R¹⁴ is unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   each U¹ is —(CR¹⁵R¹⁶)—, wherein each R¹⁵ and R¹⁶ are independently selected from hydrogen and R²⁰;
   r is 1-5;
each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
each R⁵⁰ is independently selected from unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
each R⁵¹ is independently selected from hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
or two R⁵¹ on the same N atom are taken together with the N atom to which they are attached to form an unsubstituted or substituted N-containing heterocycle;
wherein when any of R², R⁴, R⁵, R⁶, R¹⁰, R¹², R¹³, R¹⁴, R²⁰, R⁵⁰, and R⁵¹ are substituted, substituents on the R², R⁴, R⁵, R⁶, R¹⁰, R¹², R¹³, R¹⁴, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², C₁-C₆alkyl, C₁-C₆haloalkyl, monocyclic carbocycle, and monocyclic heterocycle; or two substituents on the same carbon atom are taken together to form a C═O or C═S;
   each R⁵² is independently selected from hydrogen, C₁-C₆alkyl, C₃-C₆cycloalkyl, phenyl, benzyl, 5-membered heteroaryl, and 6-membered heteroaryl;
   or two R⁵² groups are taken together with the N atom to which they are attached to form a N-containing heterocycle; and
   each R⁵³ is independently selected from C₁-C₆alkyl, C₃-C₆cycloalkyl, phenyl, benzyl, 5-membered heteroaryl, and 6-membered heteroaryl.

In a second aspect, disclosed herein is a compound represented by Formula (I) wherein R¹ is hydrogen, halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)₂NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, or unsubstituted or substituted carbocycle.

In a third aspect, disclosed herein is a compound represented by Formula (I) wherein R¹ is hydrogen, halogen, —CN, —OH, —OR⁵⁰, —SH, —SR⁵⁰, —NO₂, —NR⁵¹R⁵¹, or C₁-C₆alkyl.

In a fourth aspect, disclosed herein is a compound represented by Formula (I) wherein R¹ is hydrogen, halogen, —CN, or —NH₂.

In a fifth aspect, disclosed herein is a compound represented by Formula (I) wherein R¹ is hydrogen.

In a sixth aspect, disclosed herein is a compound represented by Formula (I) wherein R¹ is methyl.

In a seventh aspect, disclosed herein is a compound represented by Formula (I) wherein R¹ is as set forth in any one of aspects 1-6 and each R² is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)₂NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, or unsubstituted or substituted carbocycle.

In an eighth aspect, disclosed herein is a compound represented by Formula (I) wherein R¹ is as set forth in any one of aspects 1-6 and each R² is independently halogen, —CN, —OH, —OR⁵⁰, —NO₂, —NR⁵¹R⁵¹, —NR⁵¹S(═O)₂R⁵⁰, —S(═O)₂NR⁵¹R⁵¹, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, unsubstituted or substituted carbocycle, or unsubstituted or substituted heterocycle.

In a ninth aspect, disclosed herein is a compound represented by Formula (I) wherein R¹ is as set forth in any one of aspects 1-6 and each R² is independently —F, —Cl, —Br, —CN, —OH, —OR⁵⁰, —NR⁵¹R⁵¹, —C(═O)NR⁵¹R⁵¹, —NR⁵¹C(═O)R⁵⁰, or unsubstituted or substituted C₁-C₆alkyl.

In a tenth aspect, disclosed herein is a compound represented by Formula (I) wherein R¹ is as set forth in any one of aspects 1-6 and each R² is independently —F, —Cl, —Br, —CN, —OH, —OMe, —NH₂, —NMe₂, or C₁-C₆alkyl.

In an eleventh aspect, disclosed herein is a compound represented by Formula (I) wherein R¹ is as set forth in any one of aspects 1-6 and each R² is independently —Cl or —NH₂.

In a twelfth aspect, disclosed herein is a compound represented by Formula (I) wherein R¹ is as set forth in any one of aspects 1-6 and R² is halogen.

In a thirteenth aspect, disclosed herein is a compound represented by Formula (I) wherein R¹ is as set forth in any one of aspects 1-6 and R² is methyl.

In a fourteenth aspect, disclosed herein is a compound represented by Formula (I) wherein R¹ is as set forth in any one of aspects 1-6 and two R² on adjacent atoms are taken together with the atoms to which they are attached to form an unsubstituted or substituted monocyclic carbocycle or unsubstituted or substituted monocyclic heterocycle. In some embodiments, two R² on adjacent atoms are taken together with the atoms to which they are attached to form an unsubstituted or substituted monocyclic phenyl or unsubstituted or substituted monocyclic 5- or 6-membered heteroaryl.

In a fifteenth aspect, disclosed herein is a compound represented by Formula (I) wherein R¹ and R² are as set forth in any one of aspects 1-14, and m is 0, 1, 2, or 3.

In a sixteenth aspect, disclosed herein is a compound represented by Formula (I) wherein R¹ and R² are as set forth in any one of aspects 1-14, and m is 1.

In a seventeenth aspect, disclosed herein is a compound represented by Formula (I) wherein R¹ and R² are as set forth in any one of aspects 1-14, and m is 2.

In an eighteenth aspect, disclosed herein is a compound represented by Formula (I) wherein R¹ is as set forth in any one of aspects 1-6 and m is 0.

In a nineteenth aspect, disclosed herein is a compound represented by Formula (I) wherein R¹, R², and m are as set forth in any one of aspects 1-18 and Ring A is unsubstituted or substituted cycloalkyl. In some embodiments, Ring A is unsubstituted or substituted monocyclic cycloalkyl. In some embodiments, ring A is unsubstituted or substituted saturated monocyclic cycloalkyl. In some embodiments, Ring A is unsubstituted or substituted C3-C₈ cycloalkyl. In some embodiments, Ring A is unsubstituted or substituted cyclopropyl, cyclobutyl, cyclopentyl, cyclohexyl, cycloheptyl, or cyclooctyl. In some embodiments, Ring A is unsubstituted or substituted unsaturated cycloalkyl. In some embodiments, Ring A is unsubstituted or substituted cyclobutenyl, cyclopentenyl, cyclohexenyl, cycloheptenyl, or cyclooctenyl. In some embodiments, Ring A is unsubstituted or substituted polycyclic cycloalkyl.

In a twentieth aspect, disclosed herein is a compound represented by Formula (I) wherein R¹, R², and m are as set forth in any one of aspects 1-18 and Ring A is unsubstituted or substituted heterocycloalkyl. In some embodiments, Ring A is unsubstituted or substituted saturated heterocycloalkyl. In some embodiments, Ring A is unsubstituted or substituted monocyclic saturated heterocycloalkyl. In some embodiments, Ring A is unsubstituted or substituted aziridinyl, azetidinyl, oxetanyl, thietanyl, pyrrolidinyl, pyrazolidinyl, imidazolidinyl, tetrahydrofuranyl, dioxolanyl, tetrahydrothiophenyl, oxathiolanyl, piperidinyl, piperazinyl, tetrahydropyranyl, dioxanyl, thianyl, dithianyl, morpholinyl, thiomorpholinyl, azepanyl, or oxazepanyl. In some embodiments, Ring A is unsubstituted or substituted piperidinyl or piperazinyl. In some embodiments, Ring A is unsubstituted or substituted unsaturated heterocycle. In some embodiments, Ring A is unsubstituted or substituted pyrrolinyl (dihydropyrrolyl), pyrazolinyl (dihydropyrazolyl), imidazolinyl (dihydroimidazolyl), triazolinyl (dihydrotriazolyl), dihydrofuranyl, dihydrothiophenyl, oxazolinyl (dihydrooxazolyl), isoxazolinyl (dihydroisoxazolyl), thiazolinyl (dihydrothiazolyl), isothiazolinyl (dihydroisothiazolyl), oxadiazolinyl (dihydrooxadiazolyl), thiadiazolinyl (dihydrothiadiazolyl), dihydropyridinyl, tetrahydropyridinyl, dihydropyridazinyl, tetrahydropyridazinyl, dihydropyrimidinyl, tetrahydropyrimidinyl, dihydropyrazinyl, tetrahydropyrazinyl, pyranyl, dihydropyranyl, thiopyranyl, dihydrothiopyranyl, dioxinyl, dihydrodioxinyl, oxazinyl, dihydrooxazineyl, thiazinyl, or dihydrothiazinyl. In some embodiments, Ring A is unsubstituted or substituted polycyclic heterocycloalkyl. In some embodiments, Ring A is unsubstituted or substituted polycyclic heterocycloalkyl which is a bridged-, fused-, or spiro-heterocycloalkyl. In some embodiments, Ring A is unsubstituted or substituted polycyclic heterocycloalkyl which is a bridged-heterocycloalkyl. In some embodiments, Ring A is unsubstituted or substituted polycyclic heterocycloalkyl which is a fused-heterocycloalkyl. In some embodiments, Ring A is unsubstituted or substituted polycyclic heterocycloalkyl which is a spiro-heterocycloalkyl.

In a twenty-first aspect, disclosed herein is a compound represented by Formula (I) wherein R¹, R², and m are as set forth in any one of aspects 1-18 and Ring A is:

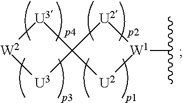

wherein:
W¹ is N, or CR²¹;
   R²¹ is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkyl(carbocycle), or unsubstituted or substituted —C₁-C₆alkyl(heterocycle);
W² is NR²², O, S, or S(═O)₂;
   R²² is hydrogen, —S(═O)₂R⁵⁰, —S(═O)R⁵⁰, —S(═O)₂NR⁵¹R⁵¹, —C(═O)R⁵⁰, —C(═O)OR⁵¹, —C(═O)NR⁵¹R⁵¹, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkyl(carbocycle), or unsubstituted or substituted —C₁-C₆alkyl(heterocycle);
each U², U^{2′}, U³, and U^{3′} is C(R²³)₂;
each R²³ is independently hydrogen or R²⁰; and
p1, p2, p3, and p4 are each independently 1-3.

In a twenty-second aspect, disclosed herein is a compound represented by Formula (I) wherein R¹, R², and m are as set forth in any one of aspects 1-18 and Ring A is as set forth in aspect 21. In some such embodiments, W¹ is N. In some such embodiments, W¹ is CR²¹. In some such embodiments, W¹ is CR²¹; and R²¹ is hydrogen, or C₁-C₆alkyl. In some such embodiments, W¹ is CH. In some such embodiments, W² is O. In some such embodiments, W² is S. In some such embodiments, W² is S(═O)₂. In some such embodiments, W² is NR²². In some such embodiments, W² is NR²²; and R²² is hydrogen, —C(═O)R⁵⁰, —C(═O)OR⁵¹, —C(═O)NR⁵¹R⁵¹, or unsubstituted or substituted C₁-C₆alkyl. In some such embodiments, W² is NR²²; and R²² is hydrogen, Boc, Fmoc, or Cbz. In some such embodiments, W² is NH. In some embodiments, W² is NBoc. In some embodiments, each R²³ is independently hydrogen, halogen, —CN, —OH, —OR⁵⁰, —NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, or unsubstituted or substituted C₁-C₆alkyl. In some embodiments, each R²³ is independently hydrogen, halogen, —OH, —OMe, —NH₂, or C₁-C₆alkyl. In some embodiments, each R²³ is hydrogen. In some embodiments, p1 is 1 to 3. In some embodiments, p1 is 1 to 2, 1 to 3, or 2 to 3. In some embodiments, p1 is 1, 2, or 3. In some embodiments, p2 is 1 to 3. In some embodiments, p2 is 1 to 2, 1 to 3, or 2 to 3. In some embodiments, p2 is 1, 2, or 3. In some embodiments, p3 is 1 to 3. In some embodiments, p3 is 1 to 2, 1 to 3, or 2 to 3. In some embodiments, p3 is 1, 2, or 3. In some embodiments, p4 is 1 to 3. In some embodiments, p4 is 1 to 2, 1 to 3, or 2 to 3. In some embodiments, p4 is 1, 2, or 3. In some embodiments, p1, p2, p3, and p4 are each independently 1 or 2. In some embodiments, W¹ is N; W² is NR²²; R²² is hydrogen, —C(═O)R⁵⁰, —C(═O)OR⁵¹, —C(═O)NR⁵¹R⁵¹, or unsubstituted or substituted C₁-C₆alkyl; each R²³ is independently hydrogen, halogen, —CN, —OH, —OR⁵⁰, —NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, and unsubstituted or substituted C₁-C₆alkyl; and p1, p2, p3, and p4 are each independently 1-2. In some such embodiments, Ring A is

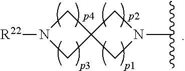

In some embodiments, Ring A is

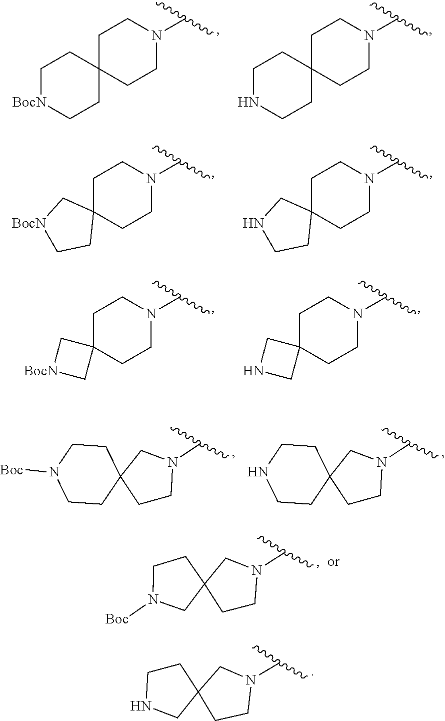

In a twenty-third aspect, disclosed herein is a compound represented by Formula (I) wherein R¹, R², and m are as set forth in any one of aspects 1-18 and Ring A is aryl or heteroaryl.

In a twenty-fourth aspect, disclosed herein is a compound represented by Formula (I) wherein R¹, R², and m are as set forth in any one of aspects 1-18 and Ring A is unsubstituted or substituted phenyl.

In a twenty-fifth aspect, disclosed herein is a compound represented by Formula (I) wherein R¹, R², and m are as set forth in any one of aspects 1-18 and Ring A is substituted phenyl.

In a twenty-sixth aspect, disclosed herein is a compound represented by Formula (I) wherein R¹, R², and m are as set forth in any one of aspects 1-18 and Ring A is substituted or unsubstituted pyridinyl.

In a twenty-seventh aspect, disclosed herein is a compound represented by Formula (I) wherein R¹, R², m, and Ring A are as set forth in any one of aspects 1-26 and R³ is unsubstituted or substituted aryl, or unsubstituted or substituted heteroaryl.

In a twenty-eighth aspect, disclosed herein is a compound represented by Formula (I) wherein R¹, R², m, and Ring A are as set forth in any one of aspects 1-26 and R³ is unsubstituted or substituted phenyl.

In a twenty-ninth aspect, disclosed herein is a compound represented by Formula (I) wherein R¹, R², m, and Ring A are as set forth in any one of aspects 1-26 and R³ is unsubstituted phenyl.

In a thirtieth aspect, disclosed herein is a compound represented by Formula (I) wherein R¹, R², m, and Ring A are as set forth in any one of aspects 1-26 and R³ is unsubstituted or substituted 5- or 6-membered heteroaryl.

In a thirty-first aspect, disclosed herein is a compound represented by Formula (I) wherein R¹, R², m, and Ring A are as set forth in any one of aspects 1-26 and R³ is unsubstituted or substituted pyrrole, furan, thiophene, imidazole, pyrazole, oxazole, isoxazole, thiazole, isothizole, triazole, oxadiazole, thiadiazole, tetrazole, pyridine, pyridazine, pyrimidine, pyrazine, or triazine. In some embodiments, R³ is unsubstituted or substituted pyridine, pyridazine, pyrimidine, pyrazine, or triazine. In some embodiments, R³ is unsubstituted or substituted pyridine. In some embodiments, R³ is unsubstituted or substituted pyridazine. In some embodiments, R³ is unsubstituted or substituted pyrimidine. In some embodiments, R³ is unsubstituted or substituted pyrazine. In some embodiments, R³ is unsubstituted or substituted pyrrole, furan, thiophene, imidazole, pyrazole, oxazole, isoxazole, thiazole, isothizole, triazole, oxadiazole, thiadiazole, or tetrazole.

In a thirty-second aspect, disclosed herein is a compound represented by Formula (I) wherein R¹, R², m, and Ring A are as set forth in any one of aspects 1-26 and R³ is unsubstituted or substituted imidazole, triazole, or pyridine.

In a thirty-third aspect, disclosed herein is a compound represented by Formula (I) wherein R¹, R², m, and Ring A are as set forth in any one of aspects 1-26 and R³ is polycyclic aryl or heteroaryl. In some embodiments, R³ is bicyclic aryl or heteroaryl. In some embodiments, R³ is naphthyl. In some embodiments, R³ is indole, isoindole, indolizine, indazole, benzimidazole, azaindole, azaindazole, purine, benzofuran, isobenzofuran, benzo[b]thiophene, benzo[c]thiophene, benzoxazole, benzisoxazole, benzthiazole, benzisothiazole, quinoline, isoquinoline, quinoxaline, phthalizine, quinazoline, cinnoline, naphthyridine, pyridopyrimidine, pyridopyrazine, or pteridine.

In a thirty-fourth aspect, disclosed herein is a compound represented by Formula (I) wherein R¹, R², m, Ring A, and R³ is as set forth in any one of aspects 1-28 and 30-33 and each R¹⁰ is independently halogen, —CN, —OH, —OR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle.

In a thirty-fifth aspect, disclosed herein is a compound represented by Formula (I) wherein R¹, R², m, Ring A, and R³ is as set forth in any one of aspects 1-28 and 30-33 and each R¹⁰ is independently —F, —Cl, —Br, —CN, —OH, —OR⁵⁰, —NR⁵¹R⁵¹, —C(═O)OR⁵¹, —C(═O)NR⁵¹R⁵¹, —NR⁵¹C(═O)R⁵⁰, or unsubstituted or substituted C₁-C₆alkyl.

In a thirty-sixth aspect, disclosed herein is a compound represented by Formula (I) wherein R¹, R², m, Ring A, and R³ is as set forth in any one of aspects 1-28 and 30-33 and each R¹⁰ is independently —OR⁵⁰ or unsubstituted or substituted C₁-C₆alkyl.

In a thirty-seventh aspect, disclosed herein is a compound represented by Formula (I) wherein R¹, R², m, Ring A, and R³ is as set forth in any one of aspects 1-28 and 30-33 and each R¹⁰ is independently —OC₁-C₆alkyl or unsubstituted or substituted C₁-C₆alkyl.

In a thirty-eight aspect, disclosed herein is a compound represented by Formula (I) wherein R¹, R², m, Ring A, and R³ is as set forth in any one of aspects 1-28 and 30-33 and each R¹⁰ is independently methyl or methoxy.

In a thirty-ninth aspect, disclosed herein is a compound represented by Formula (I) wherein R¹, R², m, Ring A, and R³ is as set forth in any one of aspects 1-28 and 30-33 and two R¹⁰ on adjacent atoms are taken together with the atoms to which they are attached to form an unsubstituted or substituted monocyclic carbocycle or unsubstituted or substituted monocyclic heterocycle. In some embodiments, two R¹⁰ on adjacent atoms are taken together with the atoms to which they are attached to form unsubstituted or substituted 5- or 6-membered monocyclic heterocycle.

In a fortieth aspect, disclosed herein is a compound represented by Formula (I) wherein R¹, R², m, Ring A, are set forth in any one of aspects 1-26 and R³ is

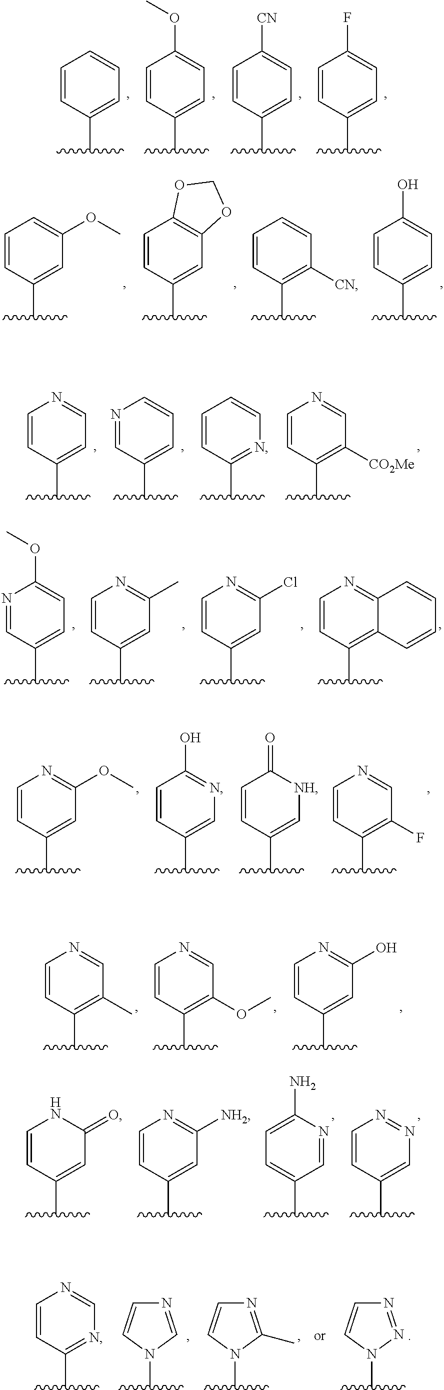

In a forty-first aspect, disclosed herein is a compound represented by Formula (I) wherein R¹, R², m, Ring A, are set forth in any one of aspects 1-26 and R³ is unsubstituted or substituted cycloalkyl, or unsubstituted or substituted heterocycloalkyl. In some embodiments, R³ is unsubstituted or substituted heterocycloalkyl. In some embodiments, R³ is unsubstituted or substituted saturated heterocycloalkyl. In some embodiments, R³ is unsubstituted or substituted monocyclic heterocycloalkyl. In some embodiments, R³ is unsubstituted or substituted monocyclic saturated heterocycloalkyl. In some embodiments, R³ is unsubstituted or substituted aziridinyl, azetidinyl, oxetanyl, thietanyl, pyrrolidinyl, pyrazolidinyl, imidazolidinyl, tetrahydrofuranyl, dioxolanyl, tetrahydrothiophenyl, sulfolanyl, oxathiolanyl, piperidinyl, piperazinyl, tetrahydropyranyl, dioxanyl, thianyl, dithianyl, morpholinyl, thiomorpholinyl, azepanyl, thiomorpholinyl dioxide, oxazepanyl, thiazepanyl, oxazocanl, and thiazocanyl. In some embodiments, R³ is unsubstituted or substituted piperidinyl or piperazinyl. In some embodiments, R³ is unsubstituted or substituted unsaturated heterocycle. In some embodiments, R³ is unsubstituted or substituted pyrrolinyl (dihydropyrrolyl), pyrazolinyl (dihydropyrazolyl), imidazolinyl (dihydroimidazolyl), triazolinyl (dihydrotriazolyl), dihydrofuranyl, dihydrothiophenyl, oxazolinyl (dihydrooxazolyl), isoxazolinyl (dihydroisoxazolyl), thiazolinyl (dihydrothiazolyl), isothiazolinyl (dihydroisothiazolyl), oxadiazolinyl (dihydrooxadiazolyl), thiadiazolinyl (dihydrothiadiazolyl), dihydropyridinyl, tetrahydropyridinyl, dihydropyridazinyl, tetrahydropyridazinyl, dihydropyrimidinyl, tetrahydropyrimidinyl, dihydropyrazinyl, tetrahydropyrazinyl, pyranyl, dihydropyranyl, thiopyranyl, dihydrothiopyranyl, dioxinyl, dihydrodioxinyl, oxazinyl, dihydrooxazineyl, thiazinyl, or dihydrothiazinyl.

In a forty-second aspect, disclosed herein is a compound represented by Formula (I) wherein R¹, R², m, Ring A, are set forth in any one of aspects 1-26 and R³ is substituted or unsubstituted morpholinyl, thiomorpholinyl, azepanyl, thiomorpholinyl dioxide, or oxazepanyl.

In a forty-third aspect, disclosed herein is a compound represented by Formula (I) wherein R¹, R², m, Ring A, are set forth in any one of aspects 1-26 and R³ is unsubstituted or substituted cycloalkyl. In some embodiments, R³ is unsubstituted or substituted monocyclic cycloalkyl. In some embodiments, R³ is unsubstituted or substituted saturated monocyclic cycloalkyl. In some embodiments, R³ is unsubstituted or substituted C₃-C₈ cycloalkyl. In some embodiments, R³ is unsubstituted or substituted cyclopropyl, cyclobutyl, cyclopentyl, cyclohexyl, cycloheptyl, or cyclooctyl. In some embodiments, R³ is unsubstituted or substituted unsaturated cycloalkyl. In some embodiments, R³ is unsubstituted or substituted cyclobutenyl, cyclopentenyl, cyclohexenyl, cycloheptenyl, or cyclooctenyl. In some embodiments, R³ is unsubstituted or substituted polycyclic cycloalkyl.

In a forty-fourth aspect, disclosed herein is a compound represented by Formula (I) wherein R¹, R², m, Ring A, are set forth in any one of aspects 1-26 and R³ is unsubstituted or substituted polycyclic heterocycloalkyl. In some embodiments, R³ is unsubstituted or substituted polycyclic heterocycloalkyl which is a bridged-, fused-, or spiro-heterocycloalkyl. In some embodiments, R³ is unsubstituted or substituted polycyclic heterocycloalkyl which is a bridged or spiro-heterocycloalkyl. In some embodiments, R³ is unsubstituted or substituted polycyclic heterocycloalkyl which is a bridged-heterocycloalkyl. In some embodiments, R³ is unsubstituted or substituted polycyclic heterocycloalkyl which is a fused-heterocycloalkyl. In some embodiments, R³ is unsubstituted or substituted polycyclic heterocycloalkyl which is a spiro-heterocycloalkyl.

In a forty-fifth aspect, disclosed herein is a compound represented by Formula (I) wherein R¹, R², m, Ring A, are set forth in any one of aspects 1-26 and R³ is unsubstituted or substituted 3- to 5-membered monocyclic heterocycloalkyl. In some embodiments, R³ is unsubstituted or substituted 3- to 5-membered monocyclic heterocycloalkyl selected from aziridinyl, azetidinyl, oxetanyl, thietanyl, pyrrolidinyl, pyrazolidinyl, imidazolidinyl, tetrahydrofuranyl, dioxolanyl, tetrahydrothiophenyl, and sulfolanyl. In some embodiments, R³ is unsubstituted or substituted 6- to 8-membered monocyclic heterocycloalkyl comprising 1 or 2 N atoms and 1 or 2 other heteroatoms selected from O or S. In some embodiments, R³ is unsubstituted or substituted 6- to 8-membered monocyclic heterocycloalkyl comprising 1 N atom and 1 other heteroatom selected from O or S. In some embodiments, R³ is unsubstituted or substituted 6- to 8-membered monocyclic heterocycloalkyl selected from morpholinyl, thiomorpholinyl, thiomorpholinyl dioxide, oxazepanyl, thiazepanyl, oxazocanyl, and thiazocanyl.

In a forty-sixth aspect, disclosed herein is a compound represented by Formula (I) wherein R¹, R², m, Ring A, are set forth in any one of aspects 1-26 and R³ is unsubstituted or substituted morpholinyl, unsubstituted or substituted thiomorpholinyl, or unsubstituted or substituted oxazepanyl.

In a forty-seventh aspect, disclosed herein is a compound represented by Formula (I) wherein R¹, R², m, Ring A, are set forth in any one of aspects 1-26 and R³ is unsubstituted morpholinyl.

In a forty-eighth aspect, disclosed herein is a compound represented by Formula (I) wherein R¹, R², m, Ring A, are set forth in any one of aspects 1-26 and R³ is unsubstituted or substituted morpholinyl, unsubstituted or substituted thiomorpholinyl, or unsubstituted or substituted oxazepanyl connected through a N atom of R³.

In a forty-ninth aspect, disclosed herein is a compound represented by Formula (I) wherein R¹, R², m, Ring A, are set forth in any one of aspects 1-26 and R³ is unsubstituted morpholinyl, unsubstituted or substituted thiomorpholinyl, or unsubstituted or substituted oxazepanyl connected through a N atom of R³.

In a fiftieth aspect, disclosed herein is a compound represented by Formula (I) wherein R¹, R², m, Ring A, are set forth in any one of aspects 1-26 and R³ is unsubstituted morpholinyl connected through a N atom of R³.

In a fifty-first aspect, disclosed herein is a compound represented by Formula (I) wherein R¹, R², m, Ring A, are set forth in any one of aspects 1-26 and R³ is as set forth in any one of aspects 41-46 and 48-49 wherein each R¹¹ is independently halogen, —CN, —OH, —OR⁵⁰, —NR⁵¹R⁵¹, —S(═O)₂R⁵⁰, —NR⁵¹S(═O)₂R⁵⁰, —S(═O)₂NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, unsubstituted or substituted carbocycle, unsubstituted or substituted heterocycle, unsubstituted or substituted —C₁-C₆alkyl(carbocycle), or unsubstituted or substituted —C₁-C₆alkyl(heterocycle). In some embodiments, each R¹¹ is independently —F, —Cl, —OH, —OR⁵⁰, —NR⁵¹R⁵¹, —S(═O)₂R⁵⁰, —C(═O)OR⁵¹, —C(═O)NR⁵¹R⁵¹, —NR⁵¹C(═O)R⁵⁰, unsubstituted or substituted C₁-C₆alkyl, unsubstituted or substituted —C₁-C₆alkyl(carbocycle). In some embodiments, when R¹¹ is substituted, substituents on R¹¹ are independently selected at each occurrence from halogen, —CN, —OH, —O—C₁-C₆alkyl, —O-benzyl, —CO₂H, —CO₂—C₁-C₆alkyl, —C(═O)—C₁-C₆alkyl, —C(═O)NR⁵²R⁵², —NR⁵²R⁵², —NHC(═O)—C₁-C₆alkyl, —NHC(═O)OBn, —NHC(═O)O—C₁-C₆alkyl, —SO₂NR⁵²R⁵², C₁-C₆alkyl, or C₁-C₆haloalkyl; and each R⁵² is independently selected from hydrogen or C₁-C₆alkyl; or two R⁵² groups are taken together with the N atom to which they are attached to form a N-containing heterocycle. In some embodiments, each R¹¹ is independently —CH₃, —OCH₃, —CH₂OH, —CH₂NH₂, —CH₂OCH₃, —S(═O)₂CH₃, —CH₂Ph, —C(═O)NH₂, or —C(═O)OCH₂CH₃. In some embodiments, two R¹¹ on the same carbon atom are taken together to form a C═O.

In a fifty-second aspect, disclosed herein is a compound represented by Formula (I) wherein R¹, R², m, Ring A, are set forth in any one of aspects 1-26 and R³ is

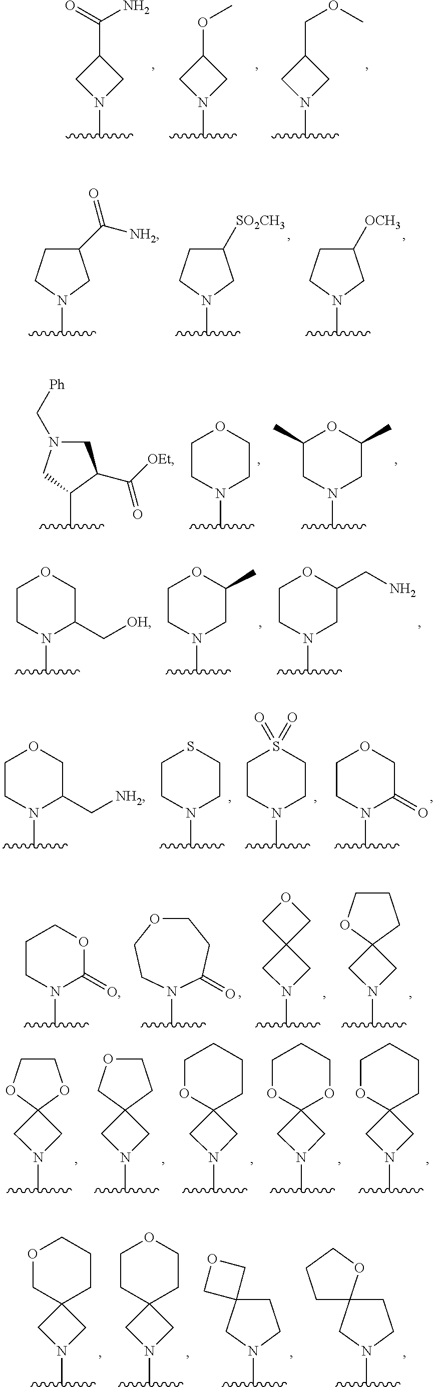

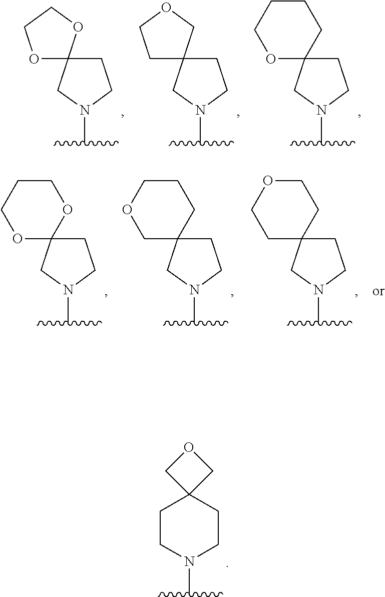

In a fifty-third aspect, disclosed herein is a compound represented by Formula (I) wherein R¹, R², m, Ring A, are set forth in any one of aspects 1-26 and R³ is

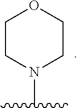 [SMILES: *N1CCOCC1]

In a fifty-fourth aspect, disclosed herein is a compound represented by Formula (I) wherein R¹, R², m, Ring A, are set forth in any one of aspects 1-26 and R³ is

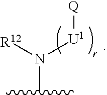

In some such embodiments R¹² is hydrogen or unsubstituted or substituted C₁-C₆alkyl. In some such embodiments, R¹² is hydrogen or C₁-C₄alkyl. In some some such embodiments, R¹² is hydrogen or methyl. In some such embodiments, R¹² is hydrogen. In some some such embodiments, R¹² is methyl. In some such embodiments, each R¹⁵ and R¹⁶ are independently hydrogen, —F, —Cl, —CN, —OH, —OR⁵⁰, —NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, unsubstituted or substituted carbocycle, unsubstituted or substituted heterocycle, unsubstituted or substituted —C₁-C₆alkyl(carbocycle), or unsubstituted or substituted —C₁-C₆alkyl(heterocycle). In some such embodiments, each R¹⁵ and R¹⁶ are independently hydrogen, —CN, —OH, —OR⁵⁰, —NR⁵¹R⁵¹, —C(═O)NR⁵¹R⁵¹, —NR⁵¹C(═O)R⁵⁰, unsubstituted or substituted C₁-C₆alkyl, or unsubstituted or substituted —C₁-C₆alkyl(carbocycle). In some such embodiments, each R¹⁵ is independently hydrogen or unsubstituted or substituted C₁-C₆alkyl; and each R¹⁶ is independently hydrogen, —CN, —OH, —OR⁵⁰, —NR⁵¹R⁵¹, —C(═O)NR⁵¹R⁵¹, —NR⁵¹C(═O)R⁵⁰, unsubstituted or substituted C₁-C₆alkyl, or unsubstituted or substituted —C₁-C₆alkyl(carbocycle). In some such embodiments, each R¹⁵ is independently hydrogen or unsubstituted or substituted C₁-C₆alkyl; and each R¹⁶ is independently —OR⁵⁰, —NR⁵¹R⁵¹, —C(═O)NR⁵¹R⁵¹, unsubstituted or substituted C₁-C₆alkyl, unsubstituted or substituted —C₁-C₂alkylene-carbocycle, or unsubstituted or substituted —C₁-C₂alkylene-heterocycle. In some such embodiments, each R¹⁵ is independently hydrogen or unsubstituted or substituted C₁-C₆alkyl; and each R¹⁶ is independently —CH₃, —CH(CH₃)₂, —CH(CH₃)CH₂CH₃, —CH₂CH(CH₃)₂, —CH₂CH₂SCH₃, —CH₂OH, —CH(OH)CH₃, —CH₂C(═O)NH₂, —CH₂CH₂C(═O)NH₂, —CH₂SH, —CH₂CH₂CH₂CH₂NH₂, —CH₂C(═O)OH, —CH₂CH₂C(═O)OH,

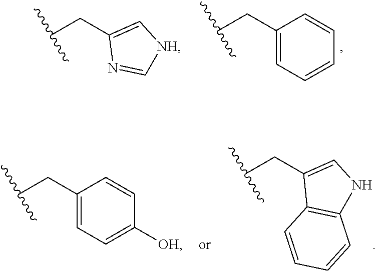 [SMILES: *Cc1c[nH]cn1 ; *Cc1ccccc1 ; *Cc1ccc(O)cc1 ; *Cc1c[nH]c2ccccc12]

In some such embodiments, r is 1 to 5. In some such embodiments, r is 1 to 2, 1 to 3, 1 to 4, 1 to 5, 2 to 3, 2 to 4, 2 to 5, 3 to 4, 3 to 5, or 4 to 5. In some such embodiments, r is 1, 2, 3, 4, or 5. In some such embodiments, r is 1, 2, or 3. In some such embodiments, Q is —OR¹³, —NR¹³R¹³, —SR¹³, —CN, —C(═O)NR¹³R¹³, or —S(═O)₂NR¹³R¹³; R¹³ is hydrogen, unsubstituted or substituted C₁-C₆alkyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; and R¹⁴ is unsubstituted or substituted C₁-C₆alkyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle. In some such embodiments, Q is —OR¹³, —CN, or —C(═O)NR¹³R¹³; and R¹³ is hydrogen or unsubstituted or substituted C₁-C₆alkyl. In some such embodiments, Q is —OCH₃, —CN, or —C(═O)NH₂. In some such embodiments, Q is —OR¹³; and R¹³ is C₁-C₆alkyl.

In a fifty-fifth aspect, disclosed herein is a compound represented by Formula (I) wherein R¹, R², m, Ring A, are set forth in any one of aspects 1-26 and R³ is

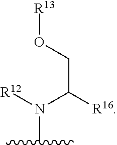

In some such embodiments, R¹⁶ is hydrogen, —F, —Cl, —CN, —OH, —OR⁵⁰, —NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, unsubstituted or substituted carbocycle, unsubstituted or substituted heterocycle, unsubstituted or substituted —C₁-C₆alkyl(carbocycle), or unsubstituted or substituted —C₁-C₆alkyl(heterocycle). In some such embodiments, R¹⁶ is hydrogen, —CN, —OH, —OR⁵⁰, —NR⁵¹R⁵¹, —C(═O)NR⁵¹R⁵¹, —NR⁵¹C(═O)R⁵⁰, unsubstituted or substituted C₁-C₆alkyl, or unsubstituted or substituted —C₁-C₆alkyl(carbocycle). In some such embodiments, R¹⁶ is hydrogen, —CN, —OH, —OR⁵⁰, —NR⁵¹R⁵¹, —C(═O)NR⁵¹R⁵¹, —NR⁵¹C(═O)R⁵⁰, unsubstituted or substituted C₁-C₆alkyl, or unsubstituted or substituted —C₁-C₆alkyl(carbocycle). In some such embodiments, R¹⁶ is —OR⁵⁰, —NR⁵¹R⁵¹, —C(═O)NR⁵¹R⁵¹, unsubstituted or substituted C₁-C₆alkyl, unsubstituted or substituted —C₁-C₂alkylene-carbocycle, or unsubstituted or substituted —C₁-C₂alkylene-heterocycle. In some such embodiments, R¹⁶ is —CH₃, —CH(CH₃)₂, —CH(CH₃)CH₂CH₃, —CH₂CH(CH₃)₂, —CH₂CH₂SCH₃, —CH₂OH, —CH(OH)CH₃, —CH₂C(═O)NH₂, —CH₂CH₂C(═O)NH₂, —CH₂SH, —CH₂CH₂CH₂CH₂NH₂, —CH₂C(═O)OH, —CH₂CH₂C(═O)OH,

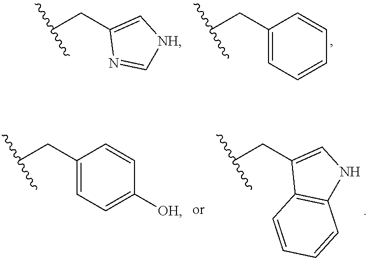

In a fifty-sixth aspect, disclosed herein is a compound represented by Formula (I) wherein R¹, R², m, Ring A, are set forth in any one of aspects 1-26 and R³ is

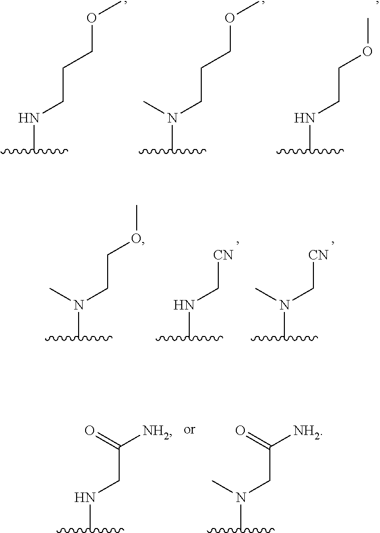

In a fifty-seventh aspect, disclosed herein is a compound represented by Formula (I) wherein R¹, R², R³, m, and A are as set forth in any one of aspects 1-56 and each R⁴ is independently R^{L} or R²⁰.

In a fifty-eighth aspect, disclosed herein is a compound represented by Formula (I) wherein R¹, R², R³, m, and A are as set forth in any one of aspects 1-56 and two R⁴ on adjacent atoms are taken together with the atoms to which they are attached to form an unsubstituted or substituted monocyclic heterocycle. In some embodiments, two R⁴ on adjacent atoms are taken together with the atoms to which they are attached to form an unsubstituted or substituted 5- or 6-membered monocyclic heterocycle. In some embodiments, two R⁴ on adjacent atoms are taken together with the atoms to which they are attached to form an unsubstituted or substituted 6-membered monocyclic heterocycle. In some embodiments, two R⁴ on adjacent atoms are taken together with the atoms to which they are attached to form a piperidine or an N-Boc piperidine.

In a fifty-ninth aspect, disclosed herein is a compound represented by Formula (I) wherein R¹, R², R³, m, and A are as set forth in any one of aspects 1-56 and each R⁴ is independently R^{L}, halogen, —CN, —OH, —OR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —C(═O)NR⁵¹R⁵¹, —NR⁵¹C(═O)R⁵⁰, unsubstituted or substituted C₁-C₆alkyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle. In some embodiments, each R⁴ is independently R^{L}, halogen, —CN, —OH, —OR⁵⁰, —NR⁵¹R⁵¹, —S(═O)₂NR⁵¹R⁵¹, —C(═O)R⁵⁰, —C(═O)OR⁵¹, —C(═O)NR⁵¹R⁵¹, unsubstituted or substituted C₁-C₆alkyl, unsubstituted or substituted heterocycle, or unsubstituted or substituted —C₁-C₆alkylene-heterocycle. In some embodiments, each R⁴ is independently R^{L}, —F, —Cl, —Br, —OR⁵⁰, —NR⁵¹R⁵¹, —S(═O)₂NR⁵¹R⁵¹, —C(═O)NR⁵¹R⁵¹, unsubstituted or substituted C₁-C₆alkyl, unsubstituted or substituted heterocycle, or unsubstituted or substituted —C₁-C₆alkylene-heterocycle.

In a sixtieth aspect, disclosed herein is a compound represented by Formula (I) wherein R¹, R², R³, m, and A are as set forth in any one of aspects 1-56 and at least one R⁴ is R^{L}. In some embodiments, one R⁴ is R^{L}.

In a sixty-first aspect, disclosed herein is a compound represented by Formula (I) wherein R¹, R², R³, m, and A are as set forth in any one of aspects 1-56 and one R⁴ is R^{L}, wherein each Y is independently unsubstituted or substituted C₁-C₂alkylene; and each R⁵ is independently halogen, —CN, —OH, —OR⁵⁰, —NR⁵¹R⁵¹, —C(═O)OR⁵¹, —C(═O)NR⁵¹R⁵¹, —NR⁵¹C(═O)R⁵⁰, unsubstituted or substituted C₁-C₆alkyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle.

In a sixty-second aspect, disclosed herein is a compound represented by Formula (I) wherein R¹, R², R³, m, and A are as set forth in any one of aspects 1-56 and one R⁴ is R^{L}, wherein each Y is independently unsubstituted or substituted C₁-C₂alkylene; and each R⁵ is independently —OR⁵⁰, —NR⁵¹R⁵¹, —C(═O)NR⁵¹R⁵¹, unsubstituted or substituted C₁-C₆alkyl, unsubstituted or substituted —C₁-C₂alkylene-carbocycle, or unsubstituted or substituted —C₁-C₂alkylene-heterocycle.

In a sixty-third aspect, disclosed herein is a compound represented by Formula (I) wherein R¹, R², R³, m, and A are as set forth in any one of aspects 1-56 and one R⁴ is R^{L}, wherein each Y is independently unsubstituted or substituted C₁-C₂alkylene; and each R⁵ is independently —CH₃, —CH(CH₃)₂, —CH(CH₃)CH₂CH₃, —CH₂CH(CH₃)₂, —CH₂CH₂SCH₃, —CH₂OH, —CH(OH)CH₃, —CH₂C(═O)NH₂, —CH₂CH₂C(═O)NH₂, —CH₂SH, —CH₂CH₂CH₂CH₂NH₂, —CH₂C(═O)OH, —CH₂CH₂C(═O)OH,

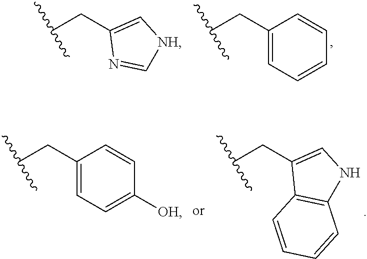

In a sixty-fourth aspect, disclosed herein is a compound represented by Formula (I) wherein R¹, R², R³, m, and A are as set forth in any one of aspects 1-56 and one R⁴ is R^{L}, wherein each Y is independently unsubstituted or substituted C₁-C₂alkylene; and each R⁵ is —CH₃. In some embodiments, one R⁴ is R^{L}, wherein each Y is independently unsubstituted C₁-C₂alkylene.

In a sixty-fifth aspect, disclosed herein is a compound represented by Formula (I) wherein R¹, R², R³, m, and A are as set forth in any one of aspects 1-56, one R⁴ is R^{L}, Y and R⁵ are set forth in any one of aspects 1-64; each Z is independently —C(═O)NR⁶— or —NR⁶C(═O)—; and each R⁶ is independently selected from hydrogen or unsubstituted or substituted C₁-C₆alkyl. In some such aspects, each R⁶ is independently selected from hydrogen or methyl. In some such aspects each Z is independently —C(═O)NH— or —NHC(═O)—.

In a sixty-sixth aspect, disclosed herein is a compound represented by Formula (I) wherein R¹, R², R³, m, and A are as set forth in any one of aspects 1-56, one R⁴ is R^{L}, Y and R⁵ are set forth in any one of aspects 1-64, wherein each Z is independently —NR⁶S(═O)₂—, —S(═O)₂NR⁶—, —C(═O)NR⁶—, or —NR⁶C(═O)—; and an R⁵ and an R⁶ on adjacent atoms are taken together with the atoms to which they are attached to form an unsubstituted or substituted monocyclic heterocycle. In some such aspects wherein each Z is independently —C(═O)NR⁶— or —NR⁶C(═O)—R⁵ and an R⁶ on adjacent atoms are taken together with the atoms to which they are attached to form an unsubstituted or substituted pyrrolidine.

In a sixty-seventh aspect, disclosed herein is a compound represented by Formula (I) wherein R¹, R², R³, m, and A are as set forth in any one of aspects 1-56, one R⁴ is R^{L}, Y, Z, R⁵ and R⁶ are set forth in any one of aspects 1-66, wherein one R⁴ is R^{L}, wherein L is unsubstituted or substituted C₁-C₆alkyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; and each R⁷ is independently halogen, —CN, —OH, —OR⁵⁰, —SH, —SR⁵⁰, —SSR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle. In some such embodiments, one R⁴ is R^{L}, wherein L is unsubstituted or substituted C₁-C₆alkyl, unsubstituted or substituted carbocycle, or unsubstituted or substituted heterocycle; and each R⁷ is independently —OR⁵⁰, —SSR⁵⁰, —NR⁵¹R⁵¹, —C(═O)R⁵⁰, —C(═O)OR¹, —C(═O)NR⁵¹R⁵¹, —NR⁵¹C(═O)R⁵⁰, unsubstituted or substituted C₁-C₆alkyl, or unsubstituted or substituted carbocycle. In some such embodiments, one R⁴ is R^{L}, wherein L is unsubstituted or substituted C₁-C₆alkyl; and each R⁷ is independently —OR⁵⁰, —SSR⁵⁰, —NR⁵¹R⁵¹, —C(═O)R⁵⁰, —C(═O)OR⁵¹, —C(═O)NR⁵¹R⁵¹, —NR⁵¹C(═O)R⁵⁰, unsubstituted or substituted C₁-C₆alkyl, or unsubstituted or substituted carbocycle. In some such embodiments, one R⁴ is R^{L}, wherein L is unsubstituted or substituted C₁-C₆alkyl; and each R⁷ is independently —OH, —OMe, —OPh, —OBn, —OC₆H₄C(═O)Me, —SS-Ph, —SS-pyridin-2-yl, —NH₂, —NHMe, —NMe2, —NHBoc, —NHCbz, —NMeBoc, —NMeCBz, —C(═O)Me, —C(═O)Ph, —C(═O)OH, —C(═O)OBn, —C(═O)NH₂, —NHC(═O)Me, C₁-C₆alkyl, or carbocycle. In some such embodiments, one R⁴ is R^{L}, wherein L is unsubstituted or substituted C₁-C₆alkyl; and each R⁷ is independently —OH, —OMe, —O-tBu, —OPh, —OBn, —OC₆H₄C(═O)Me, —SS-Ph, —SS-pyridin-2-yl, —NH₂, —NHMe, —NMe₂, —NHBoc, —NHCbz, —NHFmoc, —NMeFmoc, —NMeBoc, —NMeCBz, —C(═O)Me, —C(═O)Ph, —C(═O)OH, —C(═O)OBn, —C(═O)OtBu, —C(═O)NH₂, —NHC(═O)Me, C₁-C₆alkyl, or carbocycle. In some such embodiments, one R⁴ is R^{L}, wherein L is unsubstituted or substituted C₁-C₆alkyl; and each R⁷ is independently —O-tBu, —OC₆H₄C(═O)Me, —SS-pyridin-2-yl, —NH₂, —NHMe, —NHBoc, —NHCbz, —NHFmoc, —C(═O)Me, —C(═O)Ph, —C(═O)OH, or —C(═O)OBn. In some such embodiments, one R⁴ is R^{L}, wherein L is unsubstituted or substituted carbocycle; and each R⁷ is independently —OR⁵⁰, —NR⁵¹R⁵¹, —C(═O)R⁵⁰, —C(═O)OR⁵¹, —C(═O)NR⁵¹R⁵¹, —NR⁵¹C(═O)R⁵⁰, unsubstituted or substituted C₁-C₆alkyl, or unsubstituted or substituted carbocycle. In such some embodiments, one R⁴ is R^{L}, wherein L is unsubstituted or substituted carbocycle; and each R⁷ is independently —OH, —OMe, —OPh, —OBn, —OC₆H₄C(═O)Me, —NH₂, —NHMe, —NMe₂, —NHBoc, —NHCbz, —NMeBoc, —NMeCBz, —C(═O)Me, —C(═O)Ph, —C(═O)OH, —C(═O)OBn, —C(═O)NH₂, —NHC(═O)Me, C₁-C₆alkyl, or carbocycle. In some such embodiments, one R⁴ is R^{L}, wherein L is unsubstituted or substituted carbocycle; and each R⁷ is independently —C(═O)Me.

In a sixty-eighth aspect, disclosed herein is a compound represented by Formula (I) wherein R¹, R², R³, m, and A are as set forth in any one of aspects 1-56, Y, Z, R⁵, R⁶, R⁷, and L are set forth in any one of aspects 1-67, and s is 1 to 10. In some such embodiments, s is at least 1. In some such embodiments, s is at most 10. In some embodiments, s is 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to l0, 4 to 5, 4 to 6, 4 to 7, 4 to 8, 4 to 9, 4 to l0, 5 to 6, 5 to 7, 5 to 8, 5 to 9, 5 to l0, 6 to 7, 6 to 8, 6 to 9, 6 to l0, 7 to 8, 7 to 9, 7 to l0, 8 to 9, 8 to 10, or 9 to 10. In some such embodiments, s is 1, 2, 3, 4, 5, 6, 7, 8, 9, or 10. In some embodiments, s is 1 to 2. In some such embodiments, s is 1. In some such embodiments, s is 2.

In a sixty-ninth aspect, disclosed herein is a compound represented by Formula (I) wherein R¹, R², R³, R⁴, m, and A are as set forth in any one of aspects 1-69 wherein when R⁴ is substituted, substituents on R⁴ are independently selected at each occurrence from halogen, —CN, —OH, —O—C₁-C₆alkyl, —O-benzyl, —CO₂H, —CO₂—C₁-C₆alkyl, —C(═O)—C₁-C₆alkyl, —C(═O)NR⁵²R⁵², —NR⁵²R⁵², —NHC(═O)—C₁-C₆alkyl, —NHC(═O)OBn, —NHC(═O)O—C₁-C₆alkyl, —SO₂NR⁵²R⁵², C₁-C₆alkyl, or C₁-C₆haloalkyl; or two substituents on the same carbon atom are taken together to form a C═O; and each R⁵² is independently selected from hydrogen and C₁-C₆alkyl; or two R⁵² groups are taken together with the N atom to which they are attached to form a N-containing heterocycle.

In a seventieth aspect, disclosed herein is a compound represented by Formula (I) wherein R¹, R², R³, R⁴, m, and A are as set forth in any one of aspects 1-69 wherein when R⁴ is substituted, substituents on R⁴ are independently selected at each occurrence from halogen, —CN, —OH, —O—C₁-C₆alkyl, —CO₂H, —C(═O)NR⁵²R⁵², —NR⁵²R⁵², —NHC(═O)—C₁-C₆alkyl, C₁-C₆alkyl, or C₁-C₆haloalkyl; and each R⁵² is independently selected from hydrogen or C₁-C₆alkyl; or two R⁵² groups are taken together with the N atom to which they are attached to form a N-containing heterocycle.

In a seventy-first aspect, disclosed herein is a compound represented by Formula (I) wherein R¹, R², R³, m, and A are as set forth in any one of aspects 1-56 and each R⁴ is independently

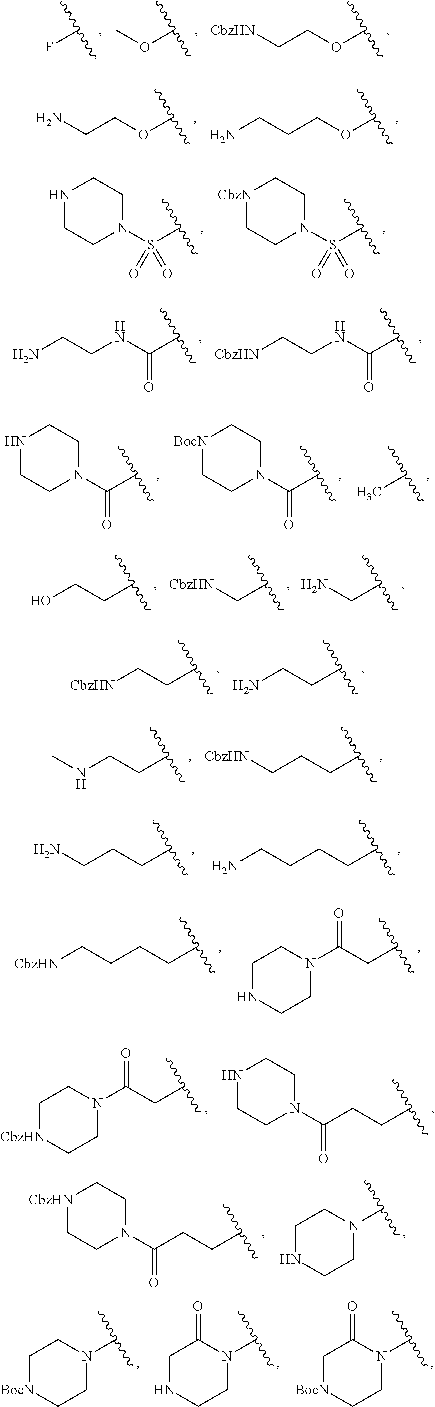

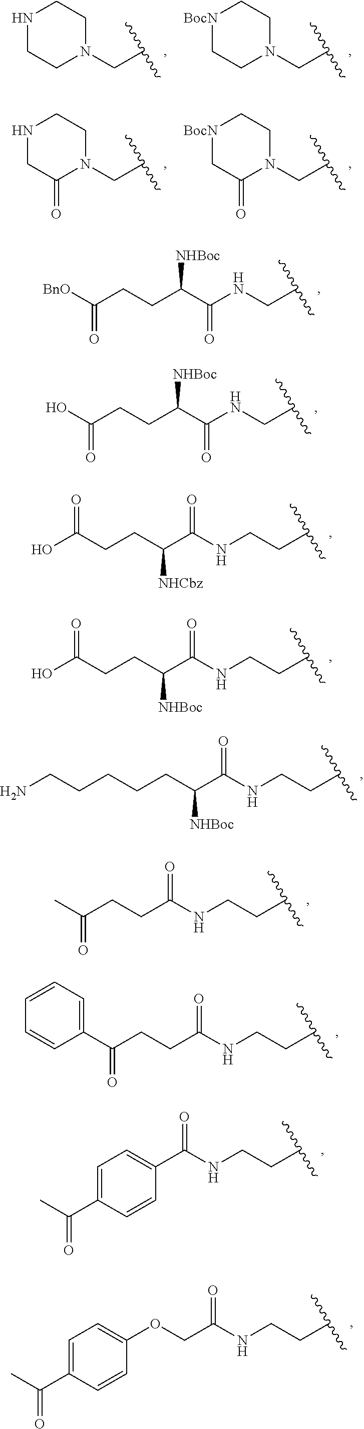

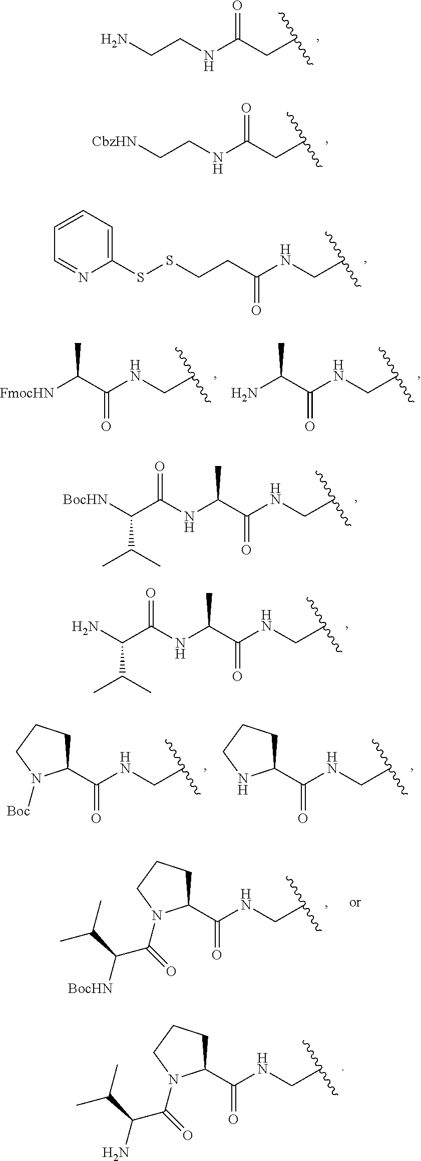

In a seventy-second aspect, disclosed herein is a compound represented by Formula (II) wherein R¹, R², R³, R⁴, and m are as set forth in any one of aspects 1-71 for Formula (I):

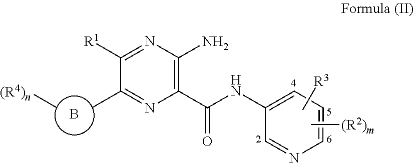

wherein: ring B is aryl or heteroaryl; and n is 0-5.

In a seventy-third aspect, disclosed herein is a compound represented by Formula (II) wherein R¹, R², R³, R⁴, and m are as set forth in any one of aspects 1-71 for Formula (I) and wherein Ring B is a monocyclic aryl or heteroaryl.

In a seventy-fourth aspect, disclosed herein is a compound represented by Formula (II) wherein R¹, R², R³, R⁴, and m are as set forth in any one of aspects 1-71 for Formula (I) and wherein Ring B is phenyl or 5- or 6-membered heteroaryl.

In a seventy-fifth aspect, disclosed herein is a compound represented by Formula (II) wherein R¹, R², R³, R⁴, and m are as set forth in any one of aspects 1-71 for Formula (I) and wherein Ring B is phenyl.

In a seventy-sixth aspect, disclosed herein is a compound represented by Formula (II) wherein R¹, R², R³, R⁴, and m are as set forth in any one of aspects 1-71 for Formula (I) and wherein Ring B is 5- or 6-membered heteroaryl.

In a seventy-seventh aspect, disclosed herein is a compound represented by Formula (II) wherein R¹, R², R³, R⁴, and m are as set forth in any one of aspects 1-71 for Formula (I) and wherein Ring B is pyrrole, furan, thiophene, imidazole, pyrazole, oxazole, isoxazole, thiazole, isothizole, triazole, oxadiazole, thiadiazole, tetrazole, pyridine, pyridazine, pyrimidine, pyrazine, or triazine.

In a seventy-eighth aspect, disclosed herein is a compound represented by Formula (II) wherein R¹, R², R³, R⁴, and m are as set forth in any one of aspects 1-71 for Formula (I) and wherein Ring B is pyridine, pyridazine, pyrimidine, pyrazine, or triazine.

In a seventy-ninth aspect, disclosed herein is a compound represented by Formula (II) wherein R¹, R², R³, R⁴, and m are as set forth in any one of aspects 1-71 for Formula (I) and wherein Ring B is pyridazine.

In an eightieth aspect, disclosed herein is a compound represented by Formula (II) wherein R¹, R², R³, R⁴, and m are as set forth in any one of aspects 1-71 for Formula (I) and wherein Ring B is pyrimidine.

In an eighty-first aspect, disclosed herein is a compound represented by Formula (II) wherein R¹, R², R³, R⁴, and m are as set forth in any one of aspects 1-71 for Formula (I) and wherein Ring B is pyrazine.

In an eighty-second aspect, disclosed herein is a compound represented by Formula (II) wherein R¹, R², R³, R⁴, and m are as set forth in any one of aspects 1-71 for Formula (I) and wherein Ring B is pyrrole, furan, thiophene, imidazole, pyrazole, oxazole, isoxazole, thiazole, isothizole, triazole, oxadiazole, thiadiazole, or tetrazole.

In an eighty-third aspect, disclosed herein is a compound represented by Formula (II) wherein R¹, R², R³, R⁴, and m are as set forth in any one of aspects 1-71 for Formula (I) and wherein Ring B is polycyclic aryl or heteroaryl.

In an eighty-fourth aspect, disclosed herein is a compound represented by Formula (II) wherein R¹, R², R³, R⁴, and m are as set forth in any one of aspects 1-71 for Formula (I) and wherein Ring B is bicyclic aryl or heteroaryl.

In an eighty-fifth aspect, disclosed herein is a compound represented by Formula (II) wherein R¹, R², R³, R⁴, and m are as set forth in any one of aspects 1-71 for Formula (I) and wherein Ring B is naphthyl.

In an eighty-sixth aspect, disclosed herein is a compound represented by Formula (II) wherein R¹, R², R³, R⁴, and m are as set forth in any one of aspects 1-71 for Formula (I) and wherein Ring B is indole, isoindole, indolizine, indazole, benzimidazole, azaindole, azaindazole, purine, benzofuran, isobenzofuran, benzo[b]thiophene, benzo[c]thiophene, benzoxazole, benzisoxazole, benzthiazole, benzisothiazole, quinoline, isoquinoline, quinoxaline, phthalizine, quinazoline, cinnoline, naphthyridine, pyridopyrimidine, pyridopyrazine, or pteridine.

In an eighty-seventh aspect, disclosed herein is a compound represented by Formula (II) wherein R¹, R², R³, R⁴, and m are as set forth in any one of aspects 1-71 for Formula (I), Ring B is as set forth in any one of aspects 72-86, and wherein n is 0 to 5.

In an eighty-eighth aspect, disclosed herein is a compound represented by Formula (II) wherein R¹, R², R³, R⁴, and m are as set forth in any one of aspects 1-71 for Formula (I), Ring B is as set forth in any one of aspects 72-86, and wherein n is 0 to 1, 0 to 2, 0 to 3, 0 to 4, 0 to 5, 1 to 2, 1 to 3, 1 to 4, 1 to 5, 2 to 3, 2 to 4, 2 to 5, 3 to 4, 3 to 5, or 4 to 5.

In an eighty-ninth aspect, disclosed herein is a compound represented by Formula (II) wherein R¹, R², R³, R⁴, and m are as set forth in any one of aspects 1-71 for Formula (I), Ring B is as set forth in any one of aspects 72-86, and wherein n is 1 to 3.

In a ninetieth aspect, disclosed herein is a compound represented by Formula (II) wherein R¹, R², R³, R⁴, and m are as set forth in any one of aspects 1-71 for Formula (I), Ring B is as set forth in any one of aspects 72-86, and wherein n is 1 to 5.

In a ninety-first aspect, disclosed herein is a compound represented by Formula (II) wherein R¹, R², R³, R⁴, and m are as set forth in any one of aspects 1-71 for Formula (I), Ring B is as set forth in any one of aspects 72-86, and wherein n is 1.

In a ninety-second aspect, disclosed herein is a compound represented by Formula (II) wherein R¹, R², R³, and m are as set forth in any one of aspects 1-71 for Formula (I), Ring B is aryl or heteroaryl (including phenyl and any of the other aryl and heteroaryl groups described herein for Ring B) and wherein two R⁴ on adjacent atoms are taken together with the atoms to which they are attached to form an unsubstituted or substituted monocyclic heterocycle. In some embodiments, two R⁴ on adjacent atoms are taken together with the atoms to which they are attached to form an unsubstituted or substituted 5- or 6-membered monocyclic heterocycle. In some embodiments, two R⁴ on adjacent atoms are taken together with the atoms to which they are attached to form an unsubstituted or substituted 6-membered monocyclic heterocycle. In some embodiments, two R⁴ on adjacent atoms are taken together with the atoms to which they are attached to form a piperidine or an N-Boc piperidine. In some embodiments,

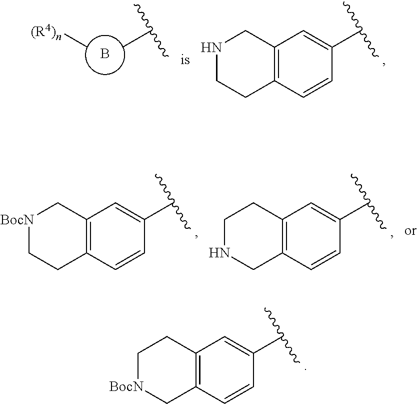

In a ninety-third aspect, disclosed herein is a compound represented by Formula (II) wherein R¹, R², R³, and m are as set forth in any one of aspects 1-71 for Formula (I), and

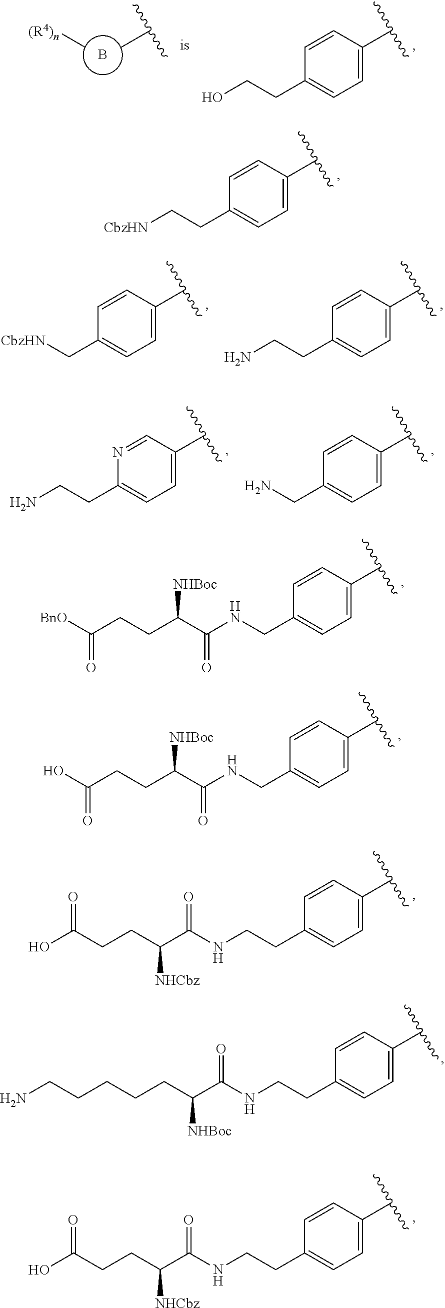

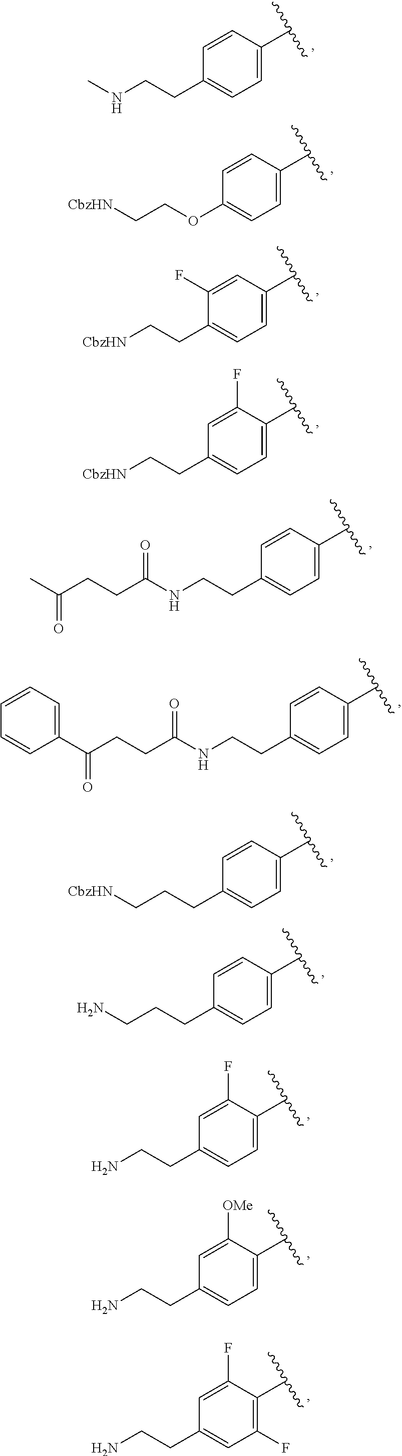

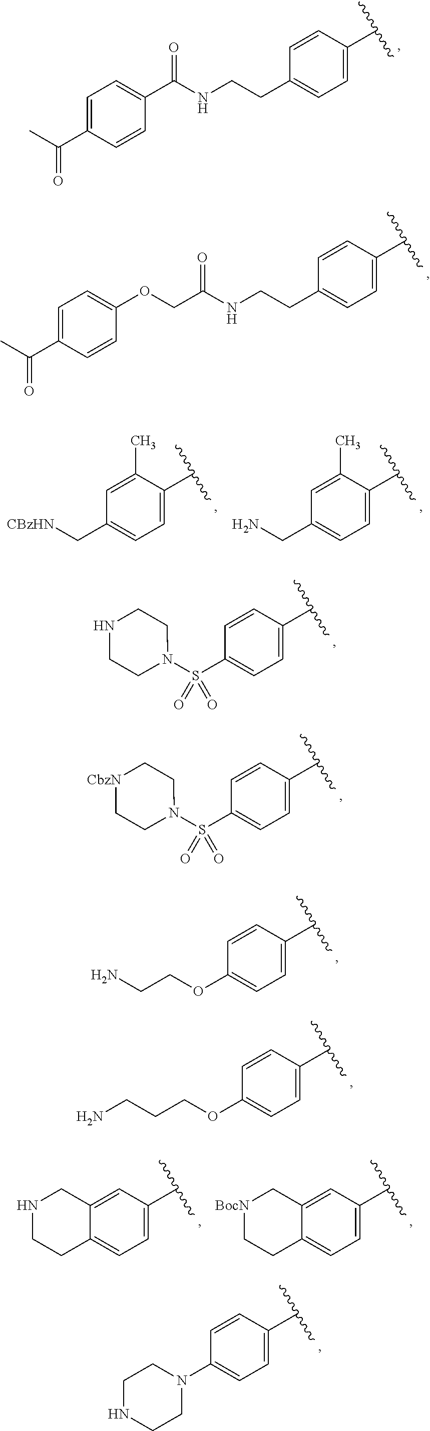

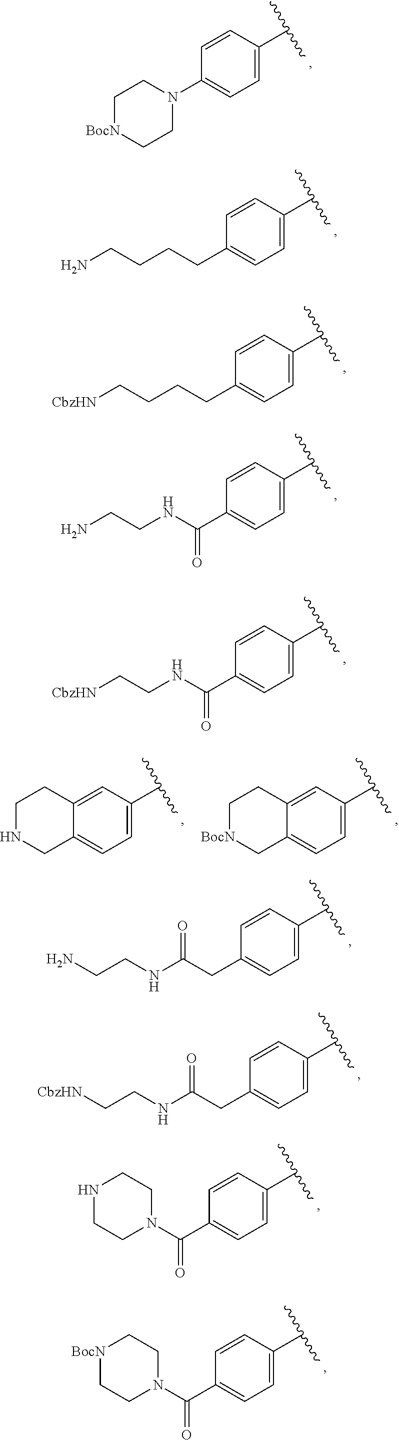

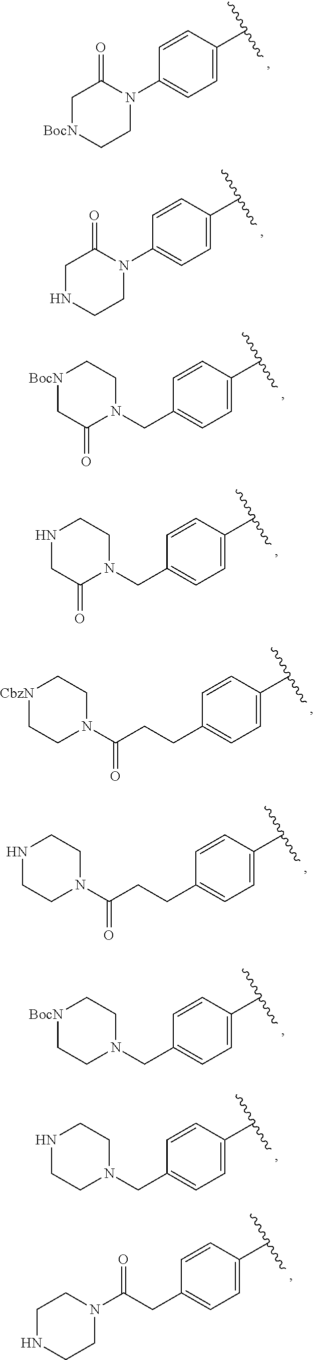

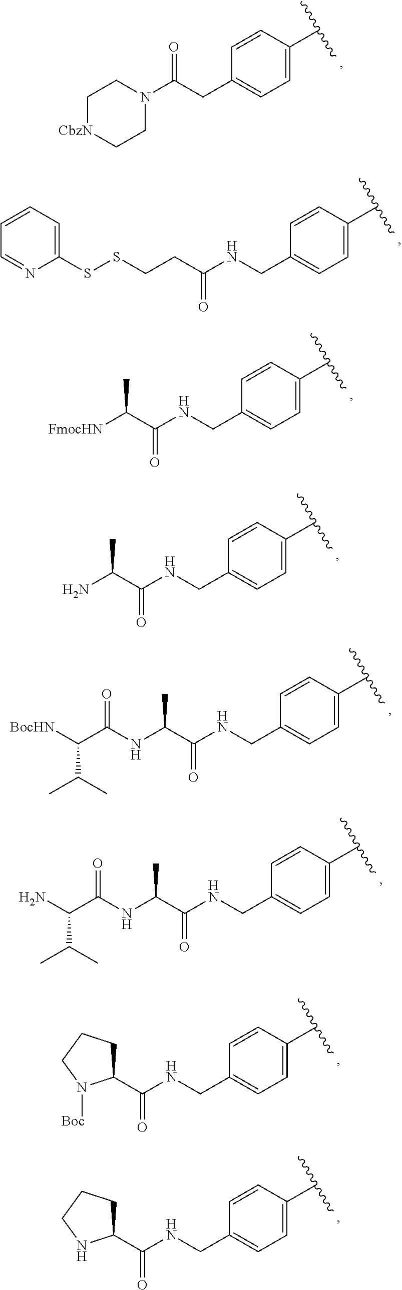

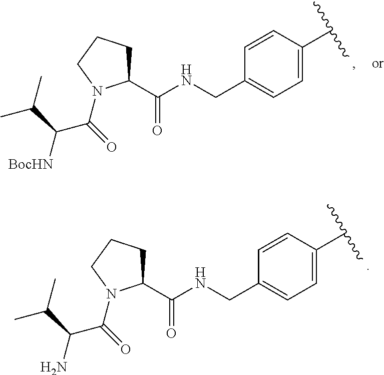 [SMILES: *c1ccc(CNC(=O)[C@@H]2CCCN2C(=O)[C@@H](NC(=O)OC(C)(C)C)C(C)C)cc1 ; *c1ccc(CNC(=O)[C@@H]2CCCN2C(=O)[C@@H](N)C(C)C)cc1]

In a ninety-fourth aspect, disclosed herein is a compound wherein R¹, R², R³, R⁴, A, n and m are as set forth in any one of aspects 1-71 for Formulas (I) and the compound of Formula (I) is represented by Formula (I-A), Formula (I-B), Formula (I-C) or Formula (I-D):

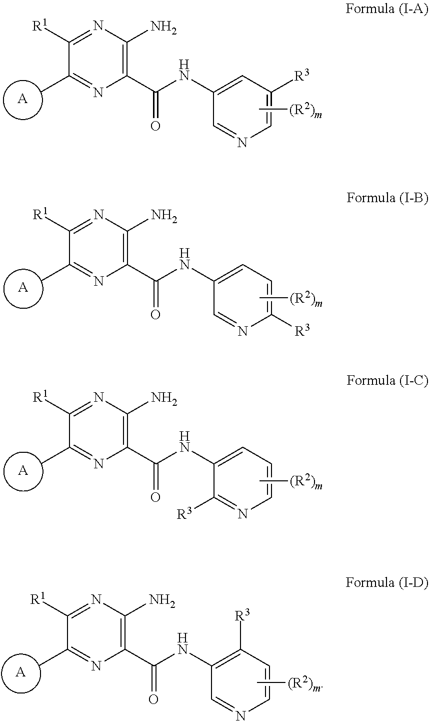

In certain embodiments, for a compound or salt of Formula (I), (I-A), (I-B), (I-C), or (I-D), when A is phenyl and R³ is morpholine, R⁴ is not alkyl substituted with —OR⁵². In certain embodiments, for a compound or salt of Formula (I), (I-A), (I-B), (I-C), or (I-D), when A is phenyl and R³ is morpholine, R⁴ is not alkyl substituted with —OH. In certain embodiments, for a compound or salt of Formula (I), (I-A), (I-B), (I-C), or (I-D), when A is phenyl and R³ is morpholine, R⁴ is not hydroxyalkyl.

In a ninety-fifth aspect, disclosed herein is a compound wherein R¹, R², R³, R⁴, B, n and m are as set forth in any one of aspects 1-93 and the compound of Formula (I) or Formula (II) is represented by Formula (II-A), Formula (II-B), Formula (II-C), or Formula (II-D):

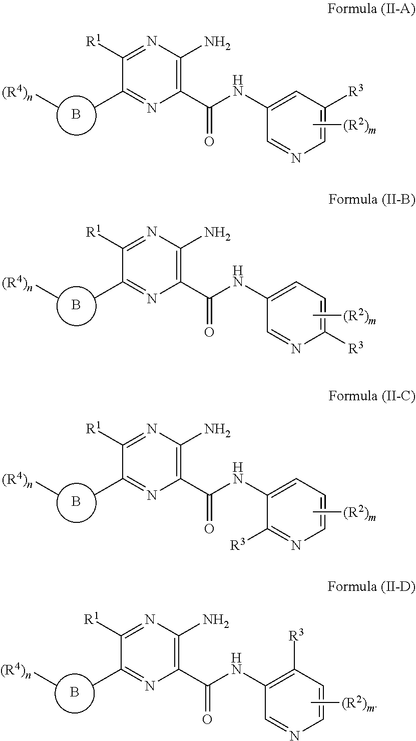

In certain embodiments, for a compound or salt of Formula (II), (IIA), (IIB), (IIC), or (IID), when B is phenyl and R³ is morpholine, R⁴ is not alkyl substituted with —OR⁵². In certain embodiments, for a compound or salt of Formula (II), (IIA), (IIB), (IIC), or (IID), when B is phenyl and R³ is morpholine, R⁴ is not alkyl substituted with —OH. In certain embodiments, for a compound or salt of Formula (II), (IIA), (IIB), (IIC), or (IID), when B is phenyl and R³ is morpholine, R⁴ is not hydroxyalkyl.

In a ninety-sixth aspect, disclosed herein is a compound wherein R¹, R², R³, A, B, n and m are as set forth in any one of aspects 1-94 for Formulas (I) and (II) and each R⁴ is independently selected from halogen, —OH, —OR⁵⁰, —NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted carbocycle, unsubstituted or substituted heterocycle, unsubstituted or substituted —C₁-C₆alkylene-carbocycle, unsubstituted or substituted —C₁-C₆alkylene-heterocycle and substituted C₁-C₆alkyl, with the proviso that C₁-C₆alkyl is substituted with —NR⁵²R⁵² and at least one of —OR⁵², —CO₂R⁵², —(C₁-C₆alkyl)-OR⁵², or (C₁-C₆alkyl)-CO₂R⁵², or two R⁴ on adjacent atoms are taken together with the atoms to which they are attached to form an unsubstituted or substituted monocyclic carbocycle or unsubstituted or substituted monocyclic heterocycle or alternatively, each R⁴ is independently selected from halogen, —OR⁵⁰, —NR⁵¹R⁵¹, —C(═O)NR⁵¹R⁵¹, —NR⁵¹C(═O)R⁵⁰, unsubstituted or substituted heterocycle, and substituted C₁-C₆alkyl with the proviso that C₁-C₆alkyl is substituted with —NR⁵²R⁵² and at least one of —OR⁵², —CO₂R⁵², —(C₁-C₆alkyl)-OR⁵², or (C₁-C₆alkyl)-CO₂R⁵²; or two R⁴ on adjacent atoms are taken together with the atoms to which they are attached to form an unsubstituted or substituted monocyclic carbocycle or unsubstituted or substituted monocyclic heterocycle.

In a ninety-seventh aspect, disclosed herein is a compound wherein R¹, R², R³, A, B, n and m are as set forth in any one of aspects 1-94 for Formulas (I) and (II) and each R⁴ is independently selected from halogen, —OR⁵⁰, —NR⁵¹R⁵¹, —C(═O)NR⁵¹R⁵¹, an unsubstituted or substituted 5- or 6-membered saturated monocyclic heterocycle containing 1 or 2 ring heteroatoms independently selected from nitrogen and oxygen, and substituted C₁-C₆alkyl with the proviso that C₁-C₆alkyl is substituted with —NR⁵²R⁵² and at least one of —OR⁵², —CO₂R⁵², —(C₁-C₆alkyl)-OR⁵², or (C₁-C₆alkyl)-CO₂R⁵²; or two R⁴ on adjacent atoms are taken together with the atoms to which they are attached to form an unsubstituted or substituted 5- or 6-membered monocyclic carbocycle or an unsubstituted or substituted 6-membered monocyclic heterocycle wherein said heterocycle contains 1 or 2 ring heteroatoms independently selected from nitrogen and oxygen.

In a ninety-eighth aspect, disclosed herein is a compound wherein R¹, R², R³, A, B, n and m are as set forth in any one of aspects 1-94 for Formulas (I) and (II) and each R⁴ is independently selected from halogen, —OR⁵⁰, —NR⁵¹R⁵¹, an unsubstituted or substituted 5- or 6-membered saturated monocyclic heterocycle containing 1 or 2 ring heteroatoms independently selected from nitrogen and oxygen, and substituted C₁-C₆alkyl with the proviso that C₁-C₆alkyl is substituted with —NR⁵²R⁵² and at least one of —OR⁵², —CO₂R⁵², —(C₁-C₆alkyl)-OR⁵², or (C₁-C₆alkyl)-CO₂R⁵²; or two R⁴ on adjacent atoms are taken together with the atoms to which they are attached to form an unsubstituted or substituted 5- or 6-membered monocyclic carbocycle or an unsubstituted or substituted 6-membered monocyclic heterocycle wherein said heterocycle contains 1 or 2 ring heteroatoms independently selected from nitrogen and oxygen.

In a ninety-ninth aspect, disclosed herein is a compound wherein R¹, R², R³, A, B, n and m are as set forth in any one of aspects 1-94 for Formulas (I) and (II) and R⁴ is as set forth in any one of aspects 95-98 and at least one R⁴ is selected from substituted C₁-C₆alkyl. In some aspects, each R⁵² of said C₁-C₆alkyl substituent is independently selected from H and C1.3 alkyl. In some aspects, each R⁵² of said C₁-C₆alkyl substituent is independently selected from H and methyl.

In a one-hundredth aspect, disclosed herein is a compound wherein R¹, R², R³, A, B, n and m are as set forth in any one of aspects 1-94 for Formulas (I) and (II) and R⁴ is as set forth in any one of aspects 95-99, at least one R⁴ is OR⁵⁰. In some aspects, R⁵⁰ of —OR⁵⁰ is independently selected from unsubstituted or substituted C₁-C₆alkyl, unsubstituted or substituted 4-, 5- or 6-membered saturated heterocycle comprising one ring heteroatom selected from nitrogen, or unsubstituted or substituted 4-, 5- or 6-membered saturated carbocycle. In some aspects, when R⁵⁰ of —OR⁵⁰ is substituted C₁-C₆alkyl, substituents on said alkyl are independently selected at each occurrence from CO₂R⁵², —OR⁵², —NR⁵²R⁵², —(C₁-C₆alkyl)-OR⁵², (C₁-C₆alkyl)-CO₂R⁵², and —(C₁-C₆alkyl)-NR⁵²R⁵². In some aspects, when R⁵⁰ of —OR⁵⁰ is substituted C₁-C₆alkyl, substituents on said C₁-C₆alkyl are independently selected at each occurrence from —CO₂R⁵², —OR⁵², and —NR⁵²R⁵². In some aspects, when R⁵⁰ of —OR⁵⁰ is substituted C₁-C₆alkyl, said C₁-C₆alkyl is substituted with —NR⁵²R⁵² and optionally one of CO₂R⁵², and —OR⁵². When R⁵⁰ of —OR⁵⁰ is substituted C₁-C₆alkyl, each R⁵² of the substituted C₁-C₆alkyl can be, for example, independently selected from H and C₁₋₃ alkyl (e.g., methyl or ethyl). When R⁵⁰ of —OR⁵⁰ is a heterocycle or carbocycle, R⁵⁰ of —OR⁵⁰ can be, for example, an unsubstituted or substituted pyrrolidine, unsubstituted or substituted piperidine, unsubstituted or substituted azetidine, or unsubstituted or substituted cyclobutyl. In some aspects when R⁵⁰ of —OR⁵⁰ is a heterocycle or carbocycle, substituents on said heterocycle and carbocycle are independently selected from CO₂R⁵², —OR⁵², —NR⁵²R⁵², or unsubstituted or substituted C₁-C₆alkyl wherein substituents on said C₁-C₆alkyl are independently selected from —OR⁵², —CO₂R⁵², or —NR⁵²R⁵². In some aspects, when R⁵⁰ of —OR⁵⁰ is a heterocycle or carbocycle, substituents on said heterocycle and carbocycle are independently selected from CO₂R⁵², —OR⁵², —NR⁵²R⁵², or unsubstituted C₁-C₆alkyl. In some aspects, when R⁵⁰ of —OR⁵⁰ is a heterocycle or carbocycle, substituents on said heterocycle and carbocycle are independently selected from —NR⁵²R⁵². Each R⁵² of the substituents on said heterocycle or carbocycle can be, for example, independently selected from H and C₁₋₃ alkyl (e.g., methyl or ethyl). In some exemplary aspects, when R⁵⁰ of —OR⁵⁰ is a heterocycle, R⁵⁰ is attached to the oxygen atom of —OR⁵⁰ at a carbon ring atom.

In a one-hundred and first aspect, disclosed herein is a compound wherein R¹, R², R³, A, B, n and m are as set forth in any one of aspects 1-94 for Formulas (I) and (II) and R⁴ is as set forth in any one of aspects 95-100 and at least one R⁴ is independently selected from a 5 or 6 membered unsubstituted or substituted saturated monocyclic heterocycle. In some aspects, when R⁴ is a substituted heterocycle, substituents on said heterocycle are independently selected from CO₂R⁵², —OR⁵², —NR⁵²R⁵², or unsubstituted or substituted C₁-C₆alkyl wherein substituents on said C₁-C₆alkyl are independently selected from —OR⁵², —CO₂R⁵², —NR⁵²R⁵² and phenyl. In some aspects, when R⁴ is a substituted heterocycle, substituents on said heterocycle are independently selected from CO₂R⁵², —OR⁵², —NR⁵²R⁵², unsubstituted C₁-C₆alkyl and phenyl. Each R⁵² of the substituents on said heterocycle can be, for example, independently selected from H and C₁₋₃ alkyl.

In a one-hundred and second aspect, disclosed herein is a compound wherein R¹, R², R³, A, B, n and m are as set forth in any one of aspects 1-94 for Formulas (I) and (II) and R⁴ is as set forth in any one of aspects 95-101 and at least one R⁴ is independently selected from —NR⁵¹R⁵¹. In some aspects, each R⁵¹ of —NR⁵¹R⁵¹ is independently selected from hydrogen, unsubstituted or substituted C₁-C₆alkyl and unsubstituted or substituted saturated N-containing heterocycle; or two R⁵¹ are taken together with the N atom to which they are attached to form an unsubstituted or substituted N-containing heterocycle. In some aspects, when R⁵¹ of —NR⁵¹R⁵¹ is a heterocycle, it is a saturated substituted or unsubstituted 5- or 6-membered heterocycle containing one ring heteroatom selected from nitrogen. In some aspects, when R⁵¹ of —NR⁵¹R⁵¹ is substituted C₁-C₆alkyl, substituents on said C₁-C₆alkyl are independently selected from OR⁵², —NR⁵²R⁵², and —CO₂R⁵² or two substituents of the same carbon atom are taken together to form C═O; and when R⁵¹ of —NR⁵¹R⁵¹ is a heterocycle, substituents on said heterocycle are independently selected from OR⁵², —NR⁵²R⁵², —CO₂R⁵², unsubstituted C₁-C₆alkyl or C₁-C₆alkyl substituted with substituents independently selected from OR⁵², —NR⁵²R⁵², —CO₂R⁵². In some aspects, when R⁵¹ of —NR⁵¹R⁵¹ is a heterocycle, it is an unsubstituted heterocycle. In some aspects, when R⁵¹ of —NR⁵¹R⁵¹ is a substituted C₁-C₆alkyl, substituents on the C₁-C₆alkyl are independently selected from OR⁵², —NR⁵²R⁵², and —CO₂R⁵². Each R⁵¹ of —NR⁵¹R⁵¹ can be, for example, independently selected from hydrogen and unsubstituted or substituted C₁-C₆alkyl wherein the substituents are independently selected from OR⁵², —NR⁵²R⁵², and —CO₂R⁵². In some aspects, R⁵² of said alkyl substituents and said heterocycle substituents are independently selected from hydrogen and C1-3 alkyl. In some aspects, R⁵² of said alkyl substituents and said heterocycle substituents are independently selected from hydrogen and methyl. In some aspects, when two R⁵¹ of —NR⁵¹R⁵¹ are taken together with the N atom to which they are attached, they form an unsubstituted or substituted 5- or 6-membered saturated N-containing heterocycle. In some aspects, when two R⁵¹ of —NR⁵¹R⁵¹ are taken together with the N atom to which they are attached, they form an unsubstituted or substituted 5- or 6-membered saturated N-containing heterocycle wherein said substituents are independently selected from —NR⁵²R⁵², unsubstituted or substituted C₁-C₆alkyl, and unsubstituted or substituted saturated monocyclic N-containing heterocycle, or two substituents of the same carbon atom are taken together to form C═O. In some aspects, substituents on said C₁-C₆alkyl are independently selected from OR⁵², NR⁵²R⁵², and CO₂R⁵² and substituents on said saturated monocyclic N-containing heterocycle are independently selected from OR⁵², NR⁵²R⁵², CO₂R⁵², and unsubstituted or substituted C₁-C₆alkyl wherein said C₁-C₆alkyl substituents are independently selected from OR⁵², NR⁵²R⁵², and CO₂R⁵². In some aspects, when two R⁵¹ of —NR⁵¹R⁵¹ are taken together with the N atom to which they are attached, they form a 6-membered substituted saturated N-containing heterocycle wherein the substituents are meta or para to ring B. In some aspects, when two R⁵¹ of —NR⁵¹R⁵¹ are taken together with the N atom to which they are attached to form a 6-membered ring, they form a substituted or unsubstituted piperazine or substituted or unsubstituted piperidine. Each R⁵² can be, for example, independently selected from hydrogen and C₁₋₃ alkyl (e.g., methyl or ethyl).

In a one-hundred and third aspect, disclosed herein is a compound wherein R¹, R², R³, A, B, n and m are as set forth in any one of aspects 1-94 for Formulas (I) and (II) and R⁴ is as set forth in any one of aspects 95-102 and at least one R⁴ is halogen (e.g., chlorine).

In a one-hundred and fourth aspect, disclosed herein is a compound wherein R¹, R², R³, B, n and m are as set forth in any one of aspects 1-94 for Formulas (I) and (II) and R⁴ is as set forth in any one of aspects 95-103 and wherein when Ring B is substituted, it is at least substituted at the para position to the pyrazine. In some aspects, when Ring B is substituted, it is substituted at the meta position to the pyrazine.

In a one-hundred and fifth aspect, disclosed herein is a compound wherein R¹, R², R³, A, B, n and m are as set forth in any one of aspects 1-94 for Formulas (I) and (II) and two R⁴ on adjacent atoms are taken together with the atoms to which they are attached to form an unsubstituted or substituted 5- or 6-membered monocyclic carbocycle or unsubstituted or substituted 6-membered monocyclic heterocycle with one or two ring heteroatoms selected from oxygen and nitrogen and substituents on said carbocycle and heterocycle are independently selected from NR⁵²R⁵², OR⁵² or —CO₂R⁵², unsubstituted C₁-C₆ alkyl and substituted C₁-C₆ alkyl with the substituents on said C₁-C₆ alkyl independently selected from NR⁵²R⁵², OR⁵² and —CO₂R⁵².

In a one-hundred and sixth aspect, disclosed herein is a compound wherein R¹, R², R³, n and m are as set forth in any one of aspects 1-94 for Formulas (I) and (II), A or B is phenyl and R⁴ is at least one of:

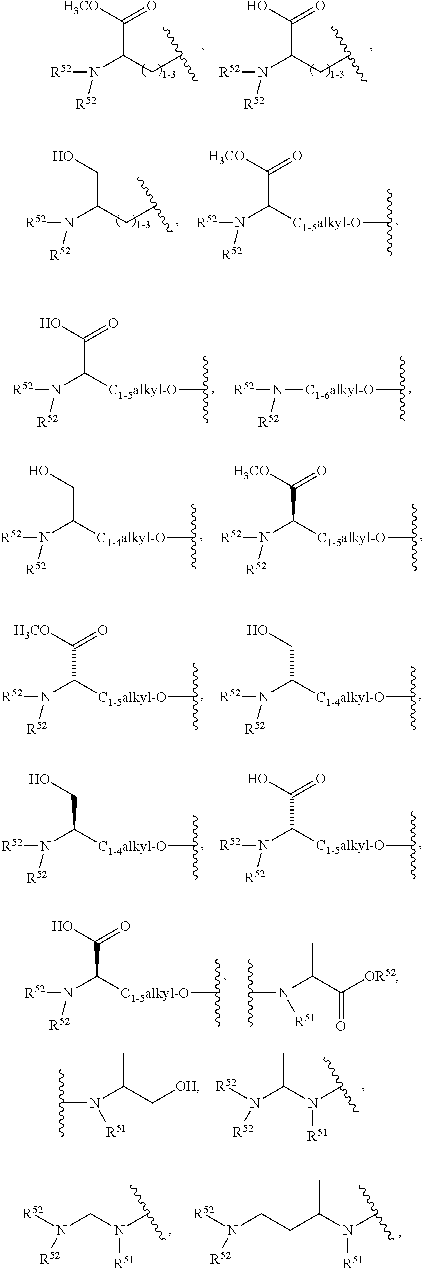

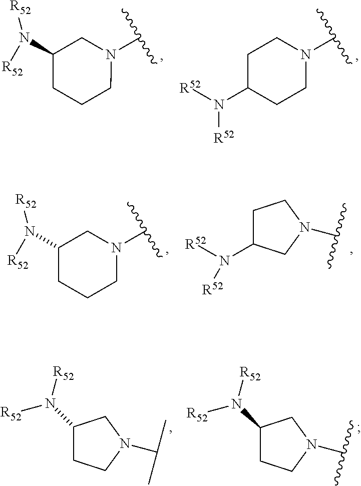

wherein R⁵¹ and R⁵² is as set forth herein for formula I or II. In some aspects, each R⁵¹ and R⁵² is independently selected from hydrogen and C₁₋₃ alkyl (e.g., methyl or ethyl).

In a one-hundred and seventh aspect, disclosed herein is a compound wherein R¹, R², R³, n and m are as set forth in any one of aspects 1-94 for Formulas (I) and (II), A or B is phenyl and R⁴ is at least one of:

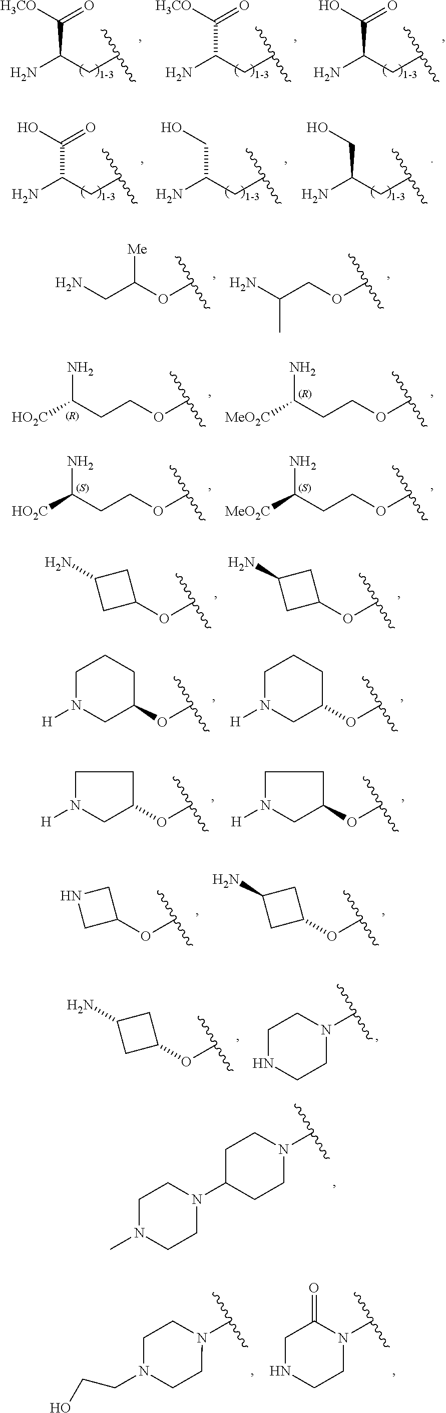

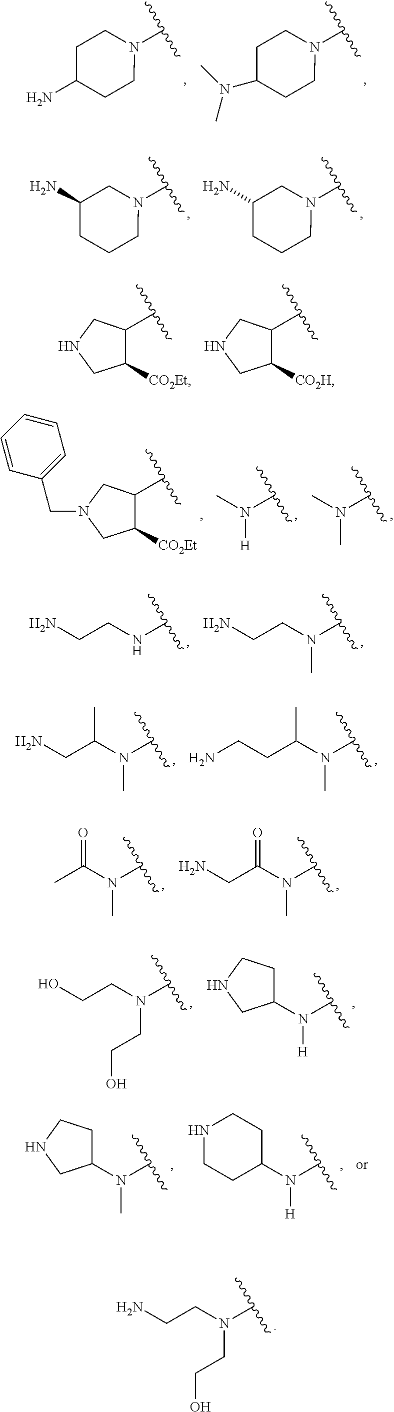

In a one-hundred and eighth aspect, disclosed herein is a compound wherein R¹, R², R³, n and m are as set forth in any one of aspects 1-94 for Formulas (I) and (II), and is

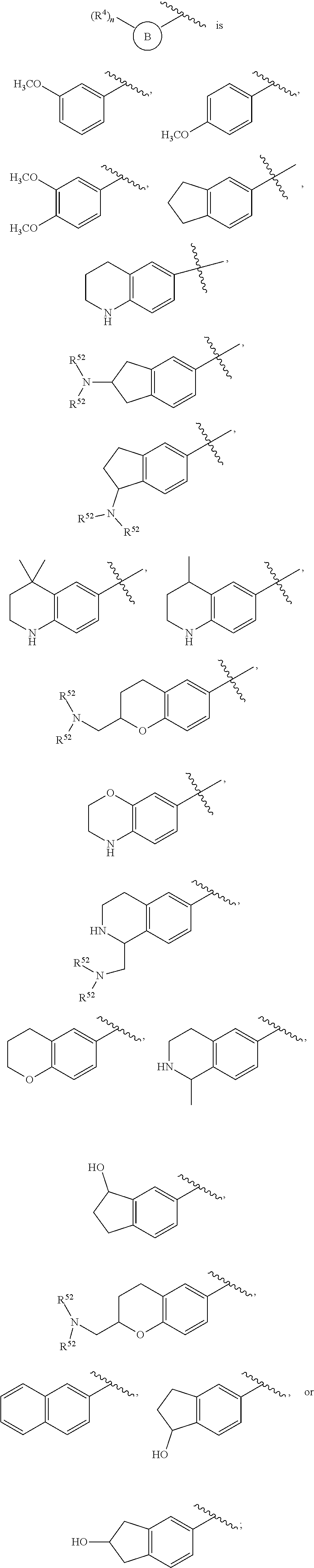

wherein R⁵² is as set forth herein for formula I or II. In some aspects, each R⁵² is independently selected from hydrogen and C₁₋₃ alkyl (e.g., methyl or ethyl).

Any combination of the groups described above for the various variables is contemplated herein. Throughout the specification, groups and substituents thereof can be chosen to provide stable moieties and compounds.

In certain embodiments, compounds described herein do not contain an electrophile. In some embodiments, an electrophile is defined as a functional group that can react to form a covalent bond with a moiety of an antibody construct such as, for example, a lysine, serine, threonine, cysteine, tyrosine, aspartic acid, glutamine, a non-natural amino acid residue, or glutamic acid residue. In some embodiments, the electrophile comprises a covalent modifier. In some embodiments, the electrophile comprises an acrylamide, an α,β-unsaturated carbonyl, a cyanopyridine, or a halo-nitrobenzene.

In some embodiments, for a compound or salt of any one of Formulas (I-A), (I-B), (I-C), (I-D), (II-A), (II-B), (II-C), and (II-D), R³ does not comprise a covalent modifier. In some embodiments, for a compound or salt of any one of Formulas (I-A), (I-B), (I-C), (I-D), (II-A), (II-B), (II-C), and (II-D), R³ does not comprise an acrylamide, an α,β-unsaturated carbonyl, a cyanopyridine, nor a halo-nitrobenzene. In some embodiments, for a compound or salt of any one of Formulas (I-A), (I-B), (I-C), (I-D), (II-A), (II-B), (II-C), and (II-D), R³ does not comprise an electrophilic group. In certain embodiments, for a compound or salt of any one of Formulas (I-A), (I-B), (I-C), (I-D), (II-A), (II-B), (II-C), and (II-D), R³ does not comprise: —CN, optionally substituted α,β-unsaturated carbonyl, and optionally substituted C₂₋₂₀ alkylene.

In certain embodiments, for a compound or salt of any one of Formulas (I-A), (I-B), (I-C), (I-D), (II-A), (II-B), (II-C), and (II-D), Ring A is not substituted with —CH₂CH₂OH. In certain embodiments, for a compound or salt of any one of Formulas (I-A), (I-B), (I-C), (I-D), (II-A), (II-B), (II-C), and (II-D), Ring A is not hydroxyalkyl. In certain embodiments, for a compound or salt of any one of Formulas (I-A), (I-B), (I-C), (I-D), (II-A), (II-B), (II-C), and (II-D), wherein Ring A is phenyl, Ring A is not substituted with —CH₂CH₂OH at the para position to the pyrazine. In certain embodiments, for a compound or salt of any one of Formulas (I-A), (I-B), (I-C), (I-D), (II-A), (II-B), (II-C), and (II-D), wherein Ring A is phenyl, Ring A is not substituted with —CH₂CH₂OH. In certain embodiments, for a compound or salt of any one of Formulas (I-A), (I-B), (I-C), (I-D), (II-A), (II-B), (II-C), and (II-D), wherein Ring A is phenyl, Ring A is not substituted with hydroxyalkyl. In certain embodiments, for a compound or salt of any one of Formulas (I-A), (I-B), (I-C), (I-D), (II-A), (II-B), (II-C), and (II-D), the compound is not

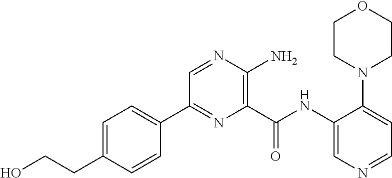 [SMILES: Nc1ncc(-c2ccc(CCO)cc2)nc1C(=O)Nc1cnccc1N1CCOCC1]

or a salt thereof.

In certain embodiments, for a compound or salt of any one of Formulas (I-A), (I-B), (I-C), (I-D), (II-A), (II-B), (II-C), and (II-D), Ring A is not substituted with a group selected from —CH₂CH₂NH₂, —CH₂NHBoc, —CH₂NH₂,

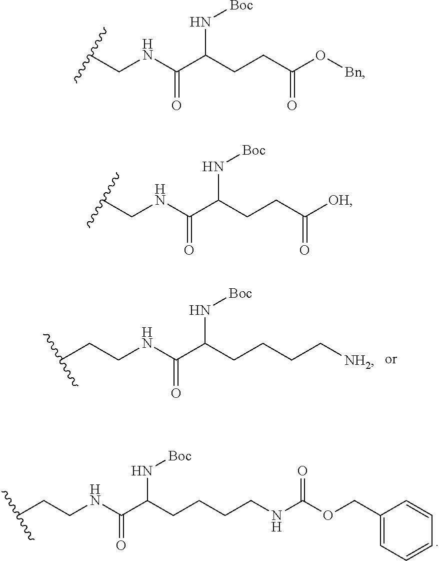 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; "or" ; *CCNC(=O)C(CCCCNC(=O)OCc1ccccc1)NC(=O)OC(C)(C)C ; "."]

In certain embodiments, for a compound or salt of any one of Formulas (I-A), (I-B), (I-C), (I-D), (II-A), (II-B), (II-C), and (II-D), Ring A is not substituted with an optionally substituted aminoalkyl group. In certain embodiments, for a compound or salt of any one of Formulas (I-A), (I-B), (I-C), (I-D), (II-A), (II-B), (II-C), and (II-D), wherein Ring A is phenyl, Ring A is not substituted with —CH₂CH₂NH₂, —CH₂NHBoc, —CH₂NH₂,

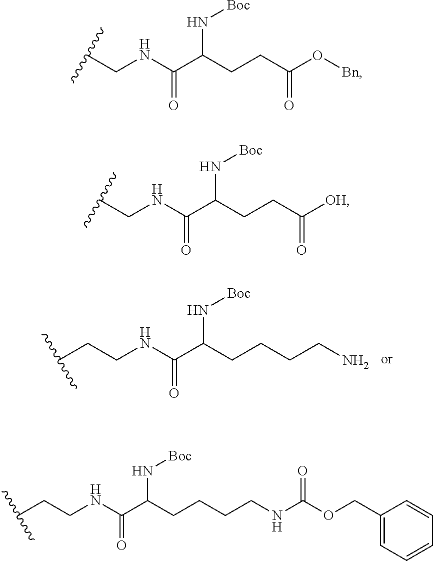 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; "or" ; *CCNC(=O)C(CCCCNC(=O)OCc1ccccc1)NC(=O)OC(C)(C)C]

any one of which is at the para position to the pyrazine. In certain embodiments, for a compound or salt of any one of Formulas (I-A), (I-B), (I-C), (I-D), (II-A), (II-B), (II-C), and (II-D), wherein Ring A is phenyl, Ring A is not substituted with —CH₂CH₂NH₂, —CH₂NHBoc, —CH₂NH₂,

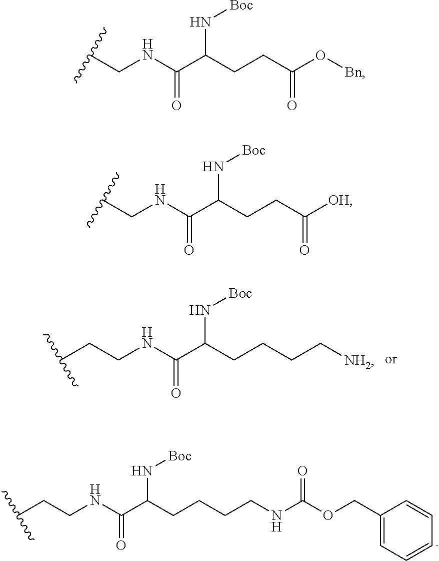 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; "or" ; *CCNC(=O)C(CCCCNC(=O)OCc1ccccc1)NC(=O)OC(C)(C)C ; "."]

In certain embodiments, for a compound or salt of any one of Formulas (I-A), (I-B), (I-C), (I-D), (II-A), (II-B), (II-C), and (II-D), wherein Ring A is phenyl, Ring A is not substituted with an optionally substituted aminoalkyl.

In certain embodiments for a compound or salt of any one of Formulas (I-A), (I-B), (I-C), (I-D), (II-A), (II-B), (II-C), and (II-D), when Ring A is substituted with an optionally substituted aminoalkyl group, Ring A is substituted with at least one other substituent.

In certain embodiments, the disclosure provides a compound represented by Formula (I-E):

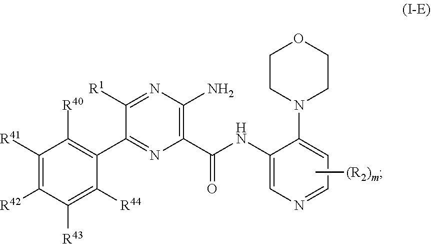

or a salt thereof, wherein:
each of R⁴⁰, R⁴¹, R⁴², R⁴³ and R⁴⁴ are independently selected from hydrogen, R^{L} and R²⁰; or two of R⁴⁰, R⁴¹, R⁴², R⁴³ and R⁴⁴ on adjacent atoms are taken together with the atoms to which they are attached to form an unsubstituted or substituted monocyclic carbocycle or unsubstituted or substituted monocyclic heterocycle wherein when said monocyclic carbocycle or said monocyclic heterocycle are substituted, substituents are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², C₁-C₆alkyl, C₁-C₆haloalkyl, aminoC₁-C₆alkyl-, Boc-aminoC₁-C₆alkyl-, Cbz-aminoC₁-C₆alkyl-, monocyclic carbocycle, and monocyclic heterocycle; or two substituents on the same carbon atom are taken together to form ═O or ═S;
   each R⁵² is independently selected from hydrogen, C₁-C₆alkyl, C₃-C₆cycloalkyl, phenyl, benzyl, 5-membered heteroaryl, and 6-membered heteroaryl;
   or two R⁵² groups are taken together with the N atom to which they are attached to form a N-containing heterocycle; and
   each R⁵³ is independently selected from C₁-C₆alkyl, C₃-C₆cycloalkyl, phenyl, benzyl, 5-membered heteroaryl, and 6-membered heteroaryl.

In certain embodiments, for a compound or salt of Formula (I-E), each of R⁴⁰, R⁴¹, R⁴², R⁴³ and R⁴⁴ are independently selected from hydrogen, R^{L} and R²⁰; or two of R⁴⁰, R⁴¹, R⁴², R⁴³ and R⁴⁴ on adjacent atoms are taken together with the atoms to which they are attached to form an unsubstituted or substituted monocyclic carbocycle or unsubstituted or substituted monocyclic heterocycle wherein when said monocyclic carbocycle or said monocyclic heterocycle are substituted, substituents are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², C₁-C₆alkyl, C₁-C₆haloalkyl, monocyclic carbocycle, and monocyclic heterocycle; or two substituents on the same carbon atom are taken together to form ═O or ═S; each R⁵² is independently selected from hydrogen, C₁-C₆alkyl, C₃-C₆cycloalkyl, phenyl, benzyl, 5-membered heteroaryl, and 6-membered heteroaryl; or two R⁵² groups are taken together with the N atom to which they are attached to form a N-containing heterocycle; and each R⁵³ is independently selected from C₁-C₆alkyl, C₃-C₆cycloalkyl, phenyl, benzyl, 5-membered heteroaryl, and 6-membered heteroaryl.

In certain embodiments, for a compound or salt of Formula (I-E), R⁴² is not —CH₂CH₂OH. In certain embodiments, for a compound or salt of Formula (I-E), R⁴² is not —CH₂CH₂OH, —CH₂CH₂NH₂, —CH₂NHBoc, —CH₂NH₂,

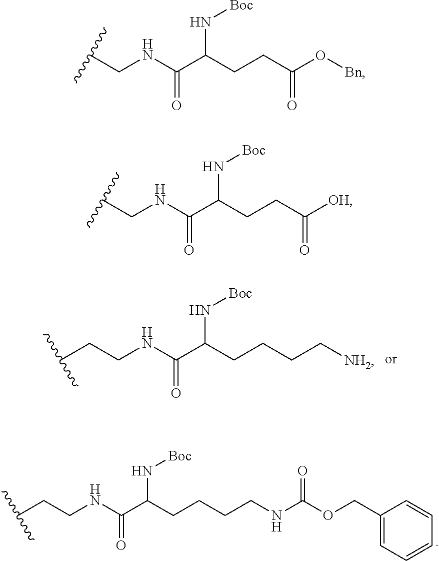 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; "or" ; *CCNC(=O)C(CCCCNC(=O)OCc1ccccc1)NC(=O)OC(C)(C)C ; "."]

In certain embodiments, for a compound or salt of Formula (I-E), R⁴² is not hydroxyalkyl. In certain embodiments, for a compound or salt of Formula (I-E), each of R⁴⁰, R⁴¹, R⁴², R⁴³ and R⁴⁴ is not —CH₂CH₂OH. In certain embodiments, for a compound or salt of Formula (I-E), each of R⁴⁰, R⁴¹, R⁴², R⁴³ and R⁴⁴ is not hydroxyalkyl.

In certain embodiments, for a compound or salt of Formula (I-E), m is 0. In certain embodiments, for a compound or salt of Formula (I-E), R¹ is hydrogen.

In certain embodiments, for a compound or salt of Formula (I-E), at least two of R⁴⁰, R⁴¹, R⁴², R⁴³ and R⁴⁴ are not hydrogen. In certain embodiments, when R⁴² is optionally substituted aminoalkyl, at least one of R⁴⁰, R⁴¹, R⁴³ and R⁴⁴ is not hydrogen. In certain embodiments, when R⁴² is hydroxyalkyl, at least one of R⁴⁰, R⁴¹, R⁴³ and R⁴⁴ is not hydrogen.

In certain embodiments, for a compound or salt of Formula (I-E), R⁴² is selected from hydrogen, halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₃-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; or R⁴² together with R⁴¹ are taken together with the atoms to which they are attached to form an unsubstituted or substituted monocyclic carbocycle or unsubstituted or substituted monocyclic heterocycle.

In certain embodiments, for a compound or salt of Formula (I-E), R⁴² is selected from hydrogen, halogen, —OH, —OR⁵⁰, —S(═O)₂R⁵⁰, —C(═O)R⁵⁰, unsubstituted or substituted C₃-C₆alkyl, unsubstituted or substituted heterocycle and unsubstituted or substituted —C₁-C₆alkylene-heterocycle; or R⁴² together with R⁴¹ are taken together with the atoms to which they are attached to form an unsubstituted or substituted monocyclic carbocycle or unsubstituted or substituted monocyclic heterocycle.

In certain embodiments, for a compound or salt of Formula (I-E), R⁴² is selected from hydrogen, —OR⁵⁰, —S(═O)₂R⁵⁰, —C(═O)R⁵⁰, unsubstituted or substituted C₃-C₆alkyl, unsubstituted or substituted —C₁-C₆alkylene-heterocycle; or R⁴² together with R⁴¹ are taken together with the atoms to which they are attached to form an unsubstituted or substituted monocyclic carbocycle or unsubstituted or substituted monocyclic heterocycle.

In certain embodiments, for a compound or salt of Formula (I-E), R⁴² is not alkyl substituted with —OR⁵². In certain embodiments, for a compound or salt of Formula (I-E), R⁴² is not alkyl substituted with —OH. In certain embodiments, for a compound or salt of Formula (I-E), R⁴² is not hydroxyalkyl. In certain embodiments, for a compound or salt of Formula (I-E), each of R⁴⁰, R⁴¹, R⁴², R⁴³ and R⁴⁴ is not alkyl substituted with —OR⁵². In certain embodiments, for a compound or salt of Formula (I-E), each of R⁴⁰, R⁴¹, R⁴², R⁴³ and R⁴⁴ is not hydroxyalkyl.

In certain embodiments, for a compound or salt of Formula (I-E), at least one of R⁴⁰, R⁴¹, R⁴², R⁴³ and R⁴⁴ is (i) —OR⁵⁰ when R⁵⁰ is C₁-C₆ alkyl substituted with —NR⁵²R⁵², —NR⁵²C(═O)R⁵³, or —NR⁵²C(═O)OR⁵²; (ii) C₁-C₆ alkyl substituted with —CO₂R⁵² or —OR⁵² and one of —NR⁵²R⁵², —NR⁵²C(═O)R⁵³, and —NR⁵²C(═O)OR⁵²; or (iii) R⁴¹ and R⁴² are taken together with the phenyl ring to which they are attached to form a substituted or unsubstituted ring system represented by:

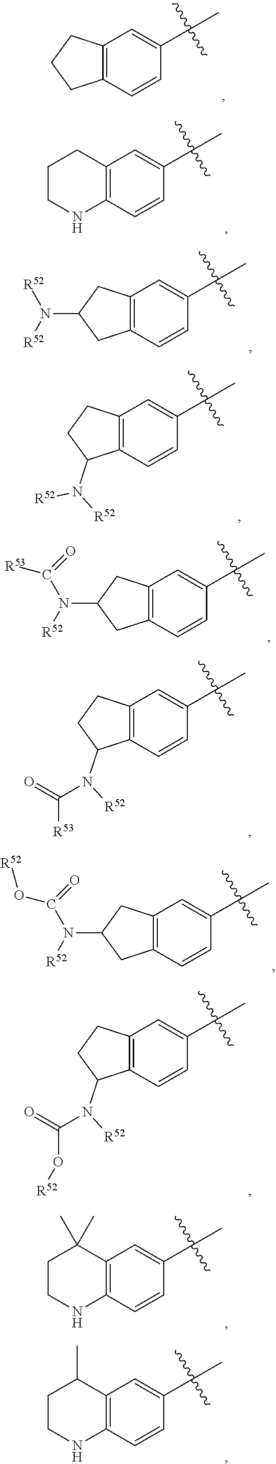

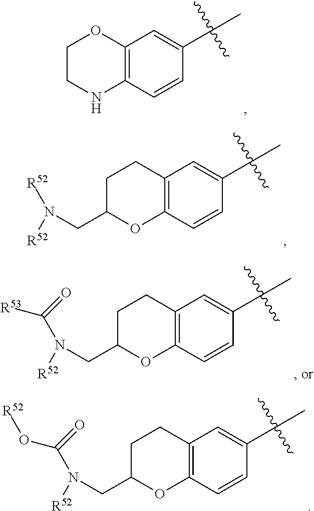

In certain embodiments, for a compound or salt of Formula (I-E), R⁴⁰, R⁴¹, R⁴³ and R⁴⁴ are independently selected from hydrogen, halogen, —OR⁵⁰, —C(═O)OR⁵¹, and unsubstituted or substituted C₁-C₆alkyl; or R⁴¹ together with R⁴² or R⁴¹ together with R⁴⁰ are taken together with the atoms to which they are attached to form an unsubstituted or substituted monocyclic carbocycle or unsubstituted or substituted monocyclic heterocycle.

In certain embodiments, for a compound or salt of Formula (I-E), R⁴⁰, R⁴¹, R⁴³ and R⁴⁴ are independently selected from hydrogen, and —OR⁵⁰; or R⁴¹ together with R⁴² are taken together with the atoms to which they are attached to form an unsubstituted or substituted monocyclic carbocycle or unsubstituted or substituted monocyclic heterocycle. In certain embodiments, for a compound or salt of Formula (I-E), R⁴⁰, R⁴¹, R⁴³ and R⁴⁴ are each hydrogen.

In certain embodiments, for a compound or salt of Formula (I-E), the compound is selected from:

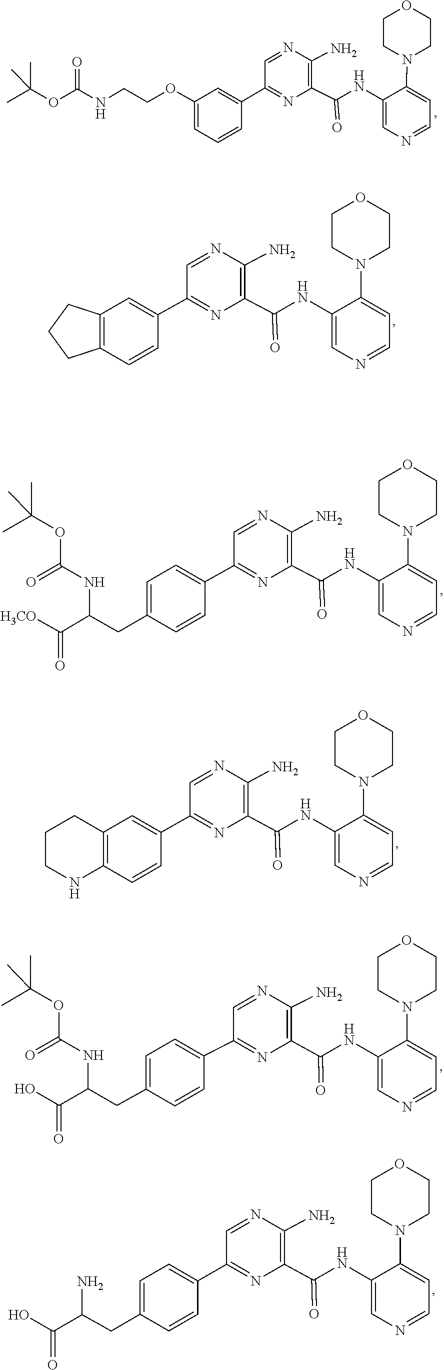 [SMILES: CC(C)(C)OC(=O)NCCOc1cccc(-c2cnc(N)c(C(=O)Nc3cnccc3N3CCOCC3)n2)c1 ; Nc1ncc(-c2ccc3c(c2)CCC3)nc1C(=O)Nc1cnccc1N1CCOCC1 ; COC(=O)C(Cc1ccc(-c2cnc(N)c(C(=O)Nc3cnccc3N3CCOCC3)n2)cc1)NC(=O)OC(C)(C)C ; Nc1ncc(-c2ccc3c(c2)CCCN3)nc1C(=O)Nc1cnccc1N1CCOCC1 ; CC(C)(C)OC(=O)NC(Cc1ccc(-c2cnc(N)c(C(=O)Nc3cnccc3N3CCOCC3)n2)cc1)C(=O)O ; Nc1ncc(-c2ccc(CC(N)C(=O)O)cc2)nc1C(=O)Nc1cnccc1N1CCOCC1]

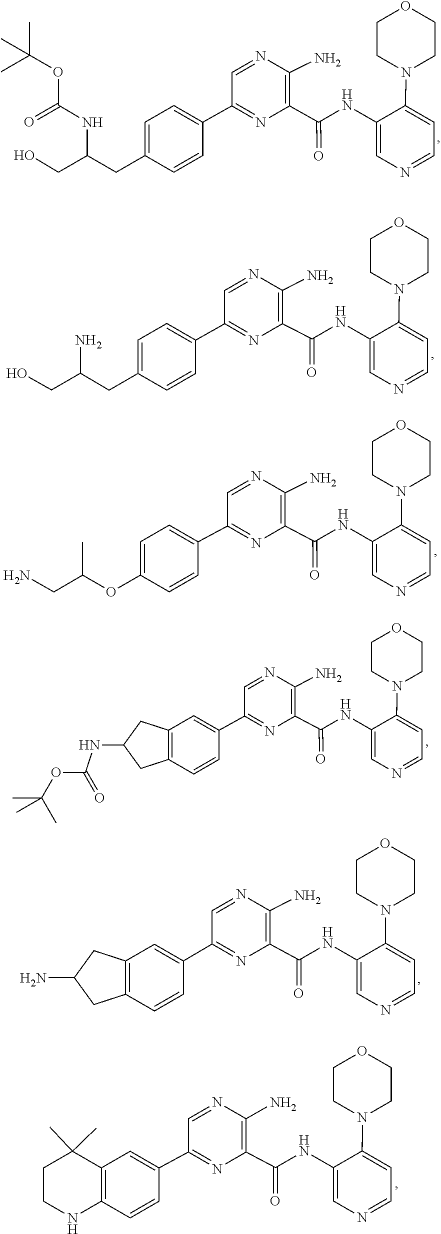

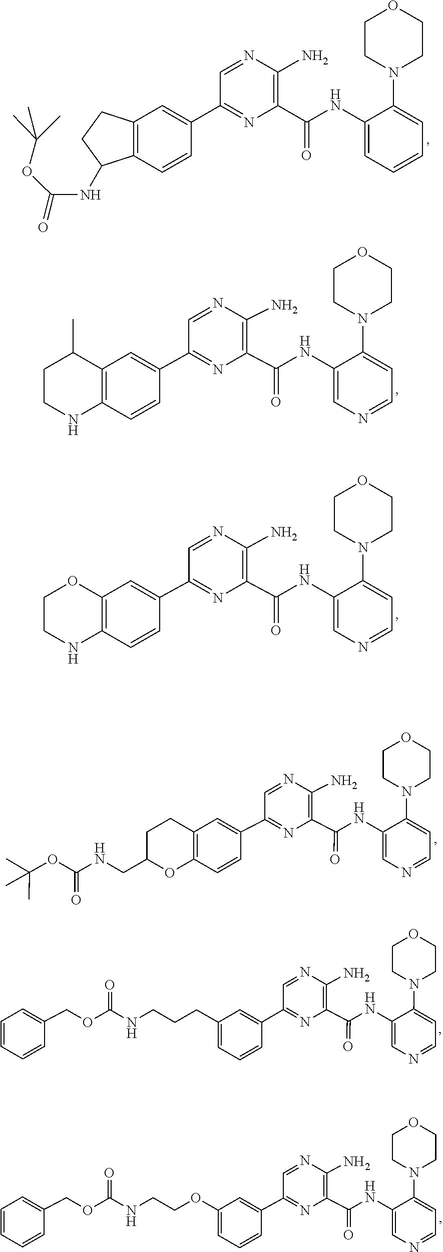 [SMILES: CC(C)(C)OC(=O)NC1CCc2cc(-c3cnc(N)c(C(=O)Nc4ccccc4N4CCOCC4)n3)ccc21 ; CC1CCNc2ccc(-c3cnc(N)c(C(=O)Nc4cnccc4N4CCOCC4)n3)cc21 ; Nc1ncc(-c2ccc3c(c2)OCCN3)nc1C(=O)Nc1cnccc1N1CCOCC1 ; CC(C)(C)OC(=O)NCC1CCc2cc(-c3cnc(N)c(C(=O)Nc4cnccc4N4CCOCC4)n3)ccc2O1 ; Nc1ncc(-c2cccc(CCCNC(=O)OCc3ccccc3)c2)nc1C(=O)Nc1cnccc1N1CCOCC1 ; Nc1ncc(-c2cccc(OCCNC(=O)OCc3ccccc3)c2)nc1C(=O)Nc1cnccc1N1CCOCC1]

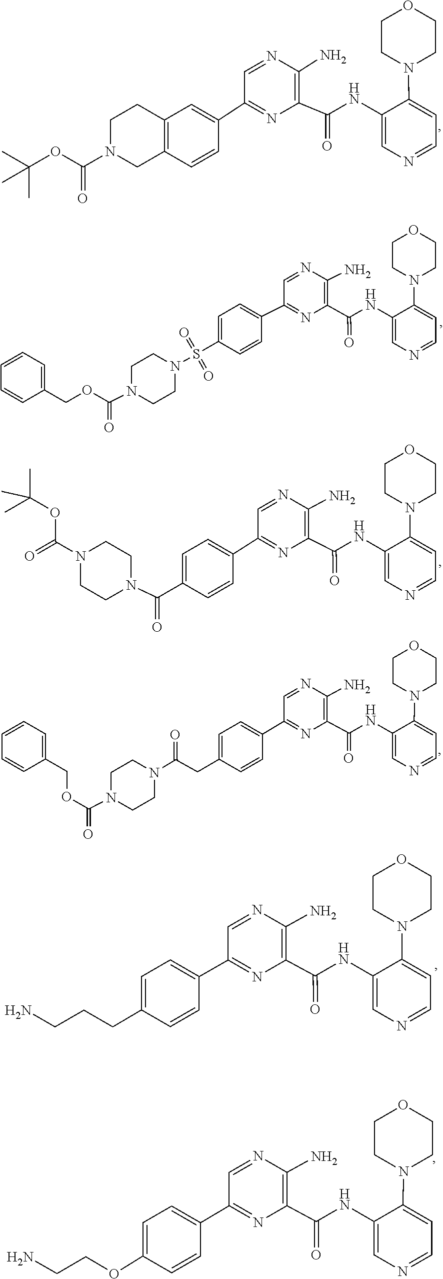 [SMILES: CC(C)(C)OC(=O)N1CCc2cc(-c3cnc(N)c(C(=O)Nc4cnccc4N4CCOCC4)n3)ccc2C1 ; Nc1ncc(-c2ccc(S(=O)(=O)N3CCN(C(=O)OCc4ccccc4)CC3)cc2)nc1C(=O)Nc1cnccc1N1CCOCC1 ; CC(C)(C)OC(=O)N1CCN(C(=O)c2ccc(-c3cnc(N)c(C(=O)Nc4cnccc4N4CCOCC4)n3)cc2)CC1 ; Nc1ncc(-c2ccc(CC(=O)N3CCN(C(=O)OCc4ccccc4)CC3)cc2)nc1C(=O)Nc1cnccc1N1CCOCC1 ; NCCCc1ccc(-c2cnc(N)c(C(=O)Nc3cnccc3N3CCOCC3)n2)cc1 ; NCCOc1ccc(-c2cnc(N)c(C(=O)Nc3cnccc3N3CCOCC3)n2)cc1]

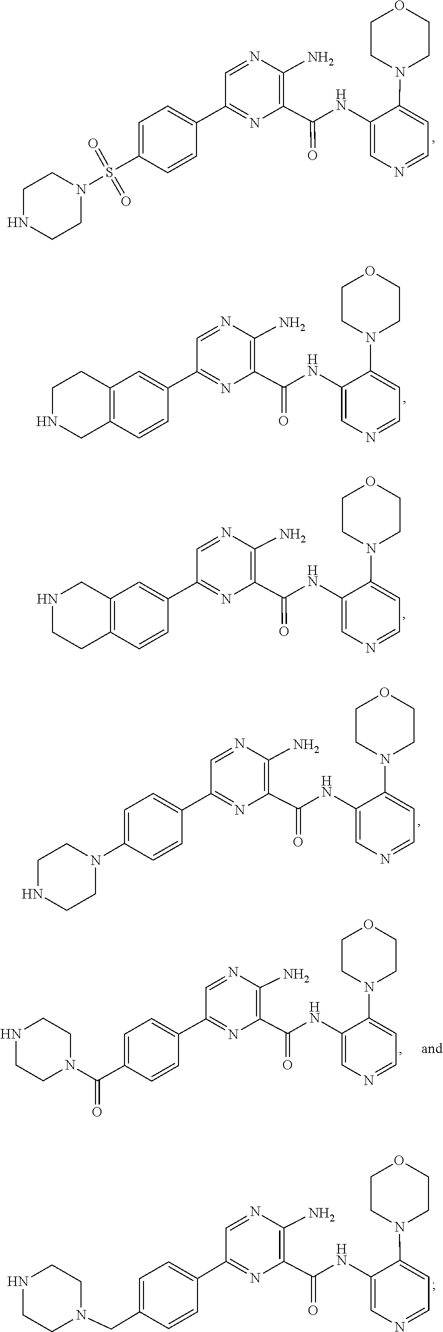

a salt of any one thereof.

In certain embodiments, exemplary compounds may include, but are not limited to, a compound or salt selected from:

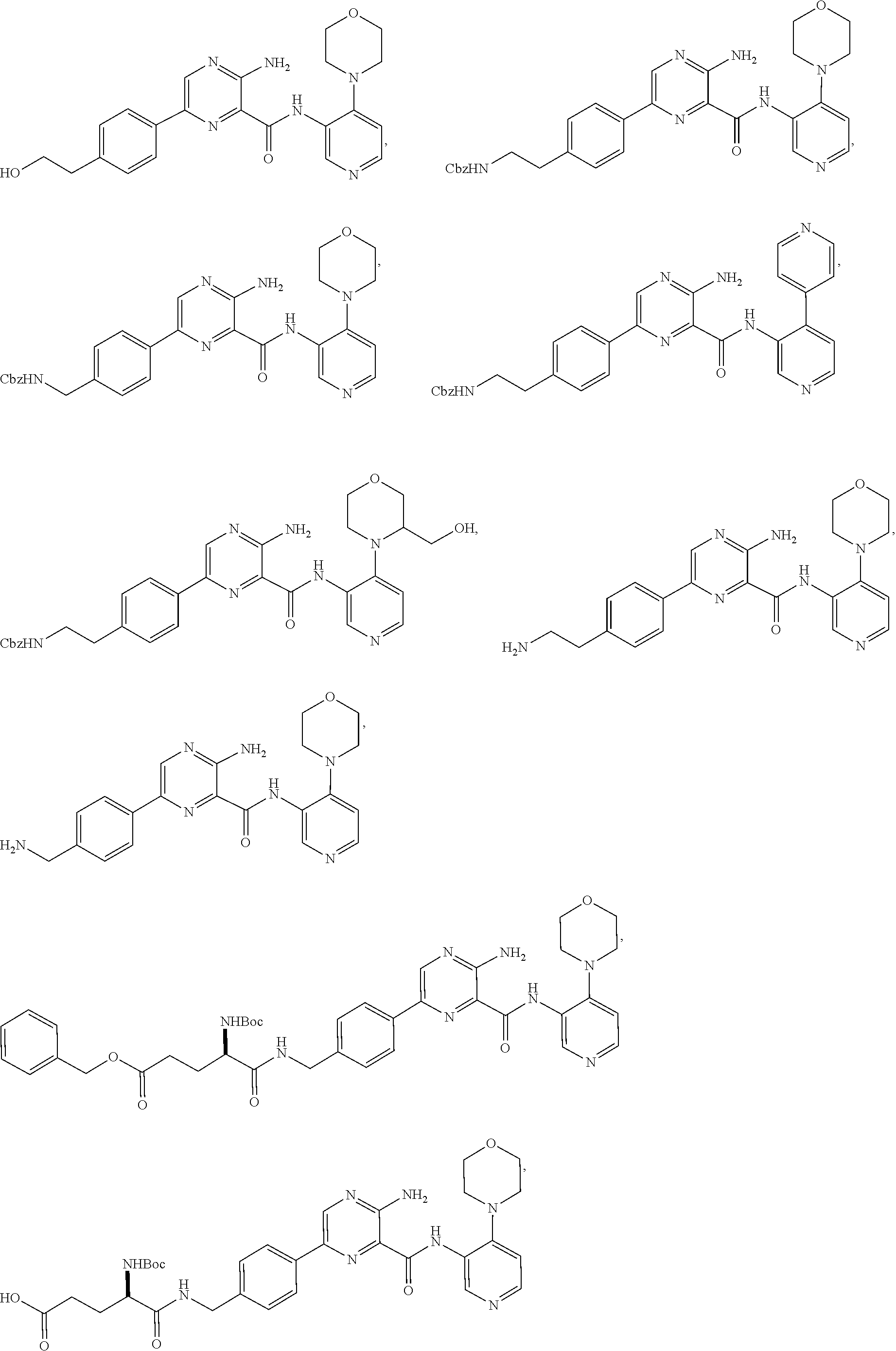

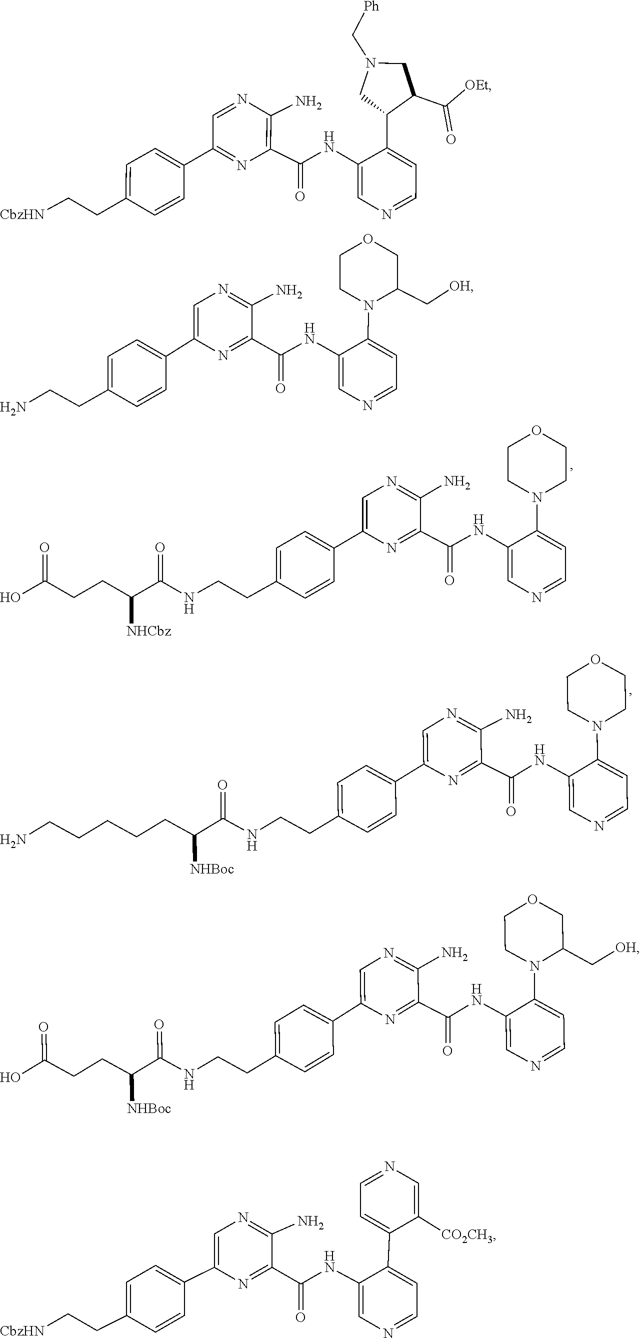

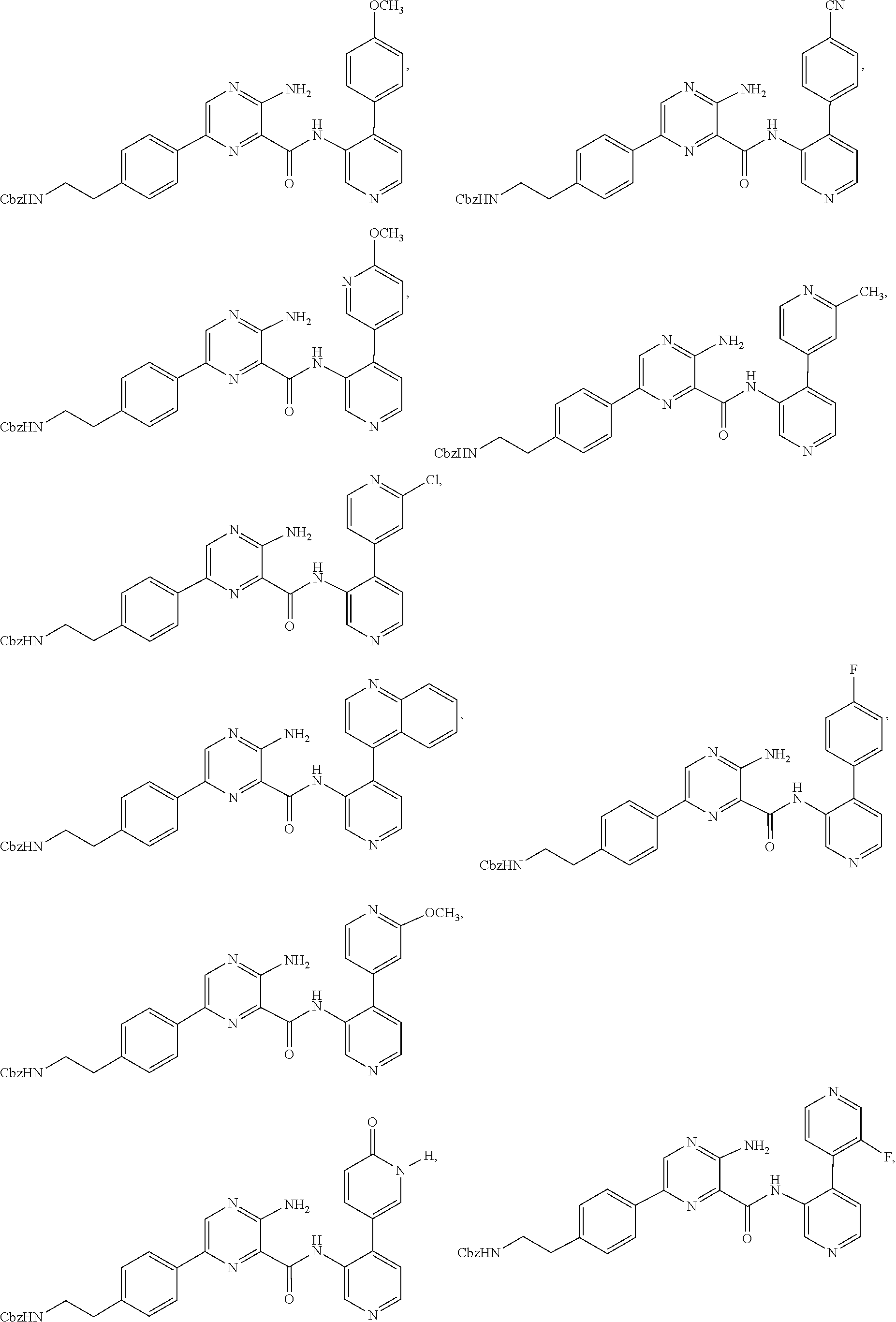

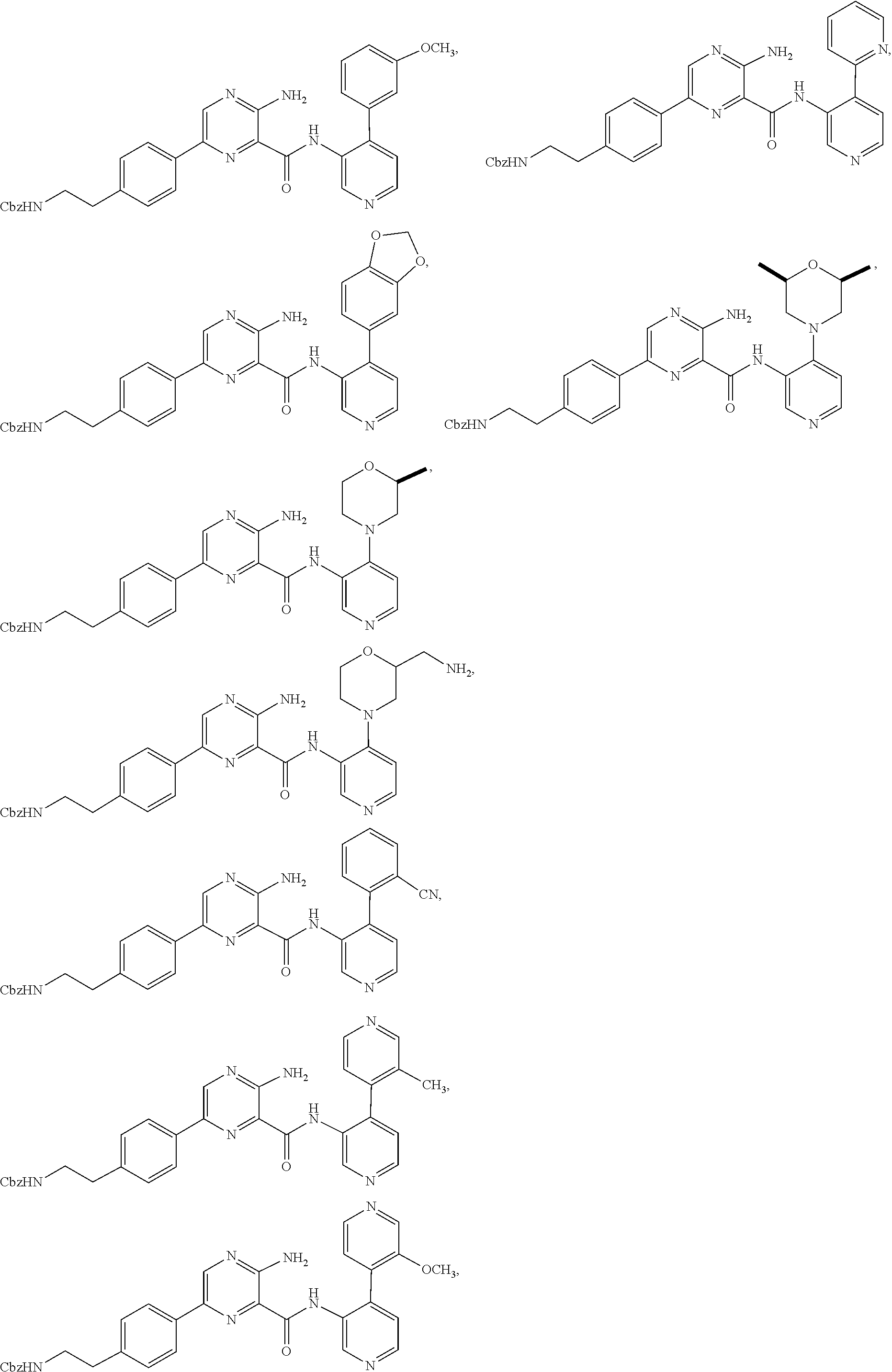

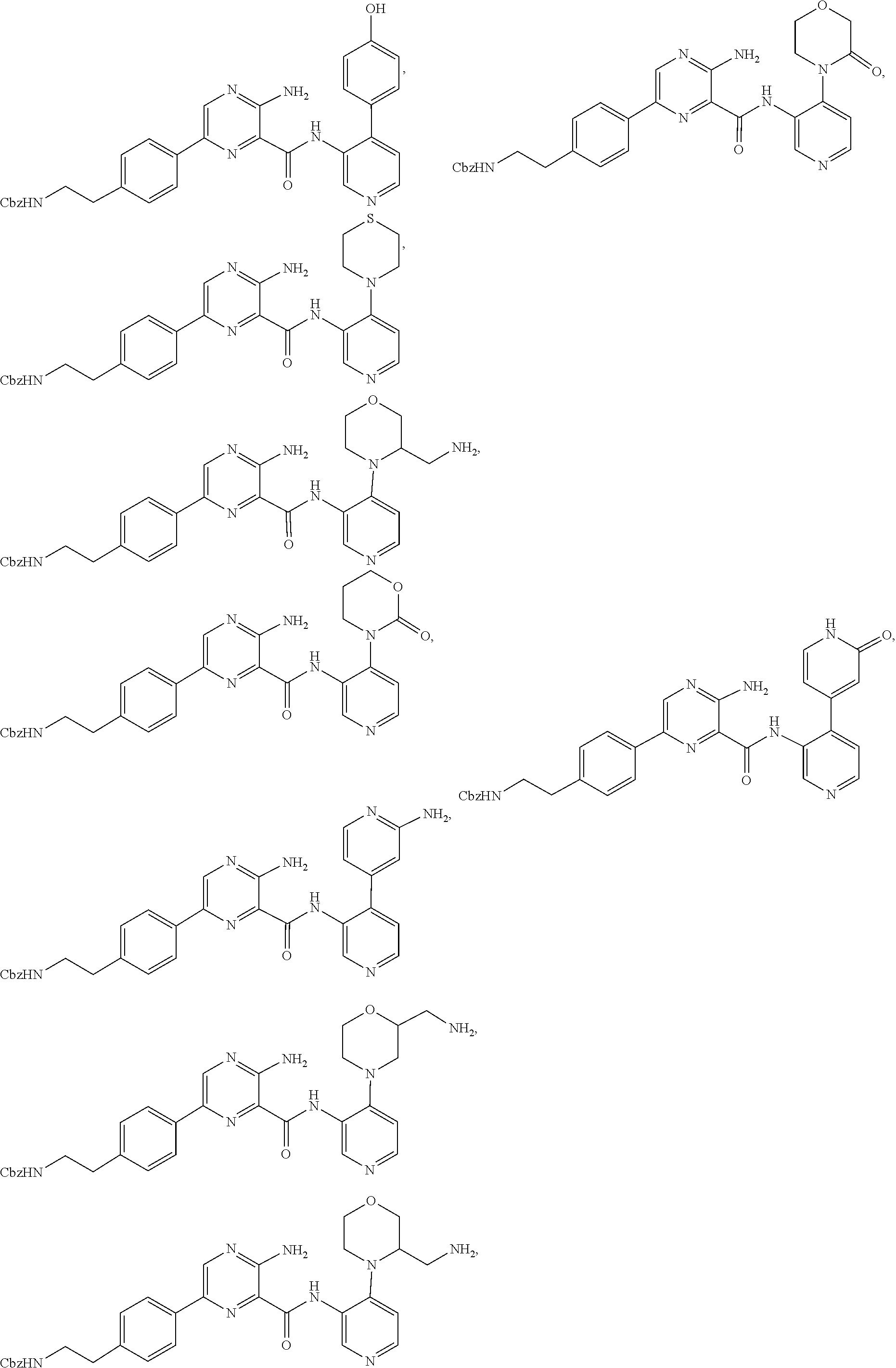

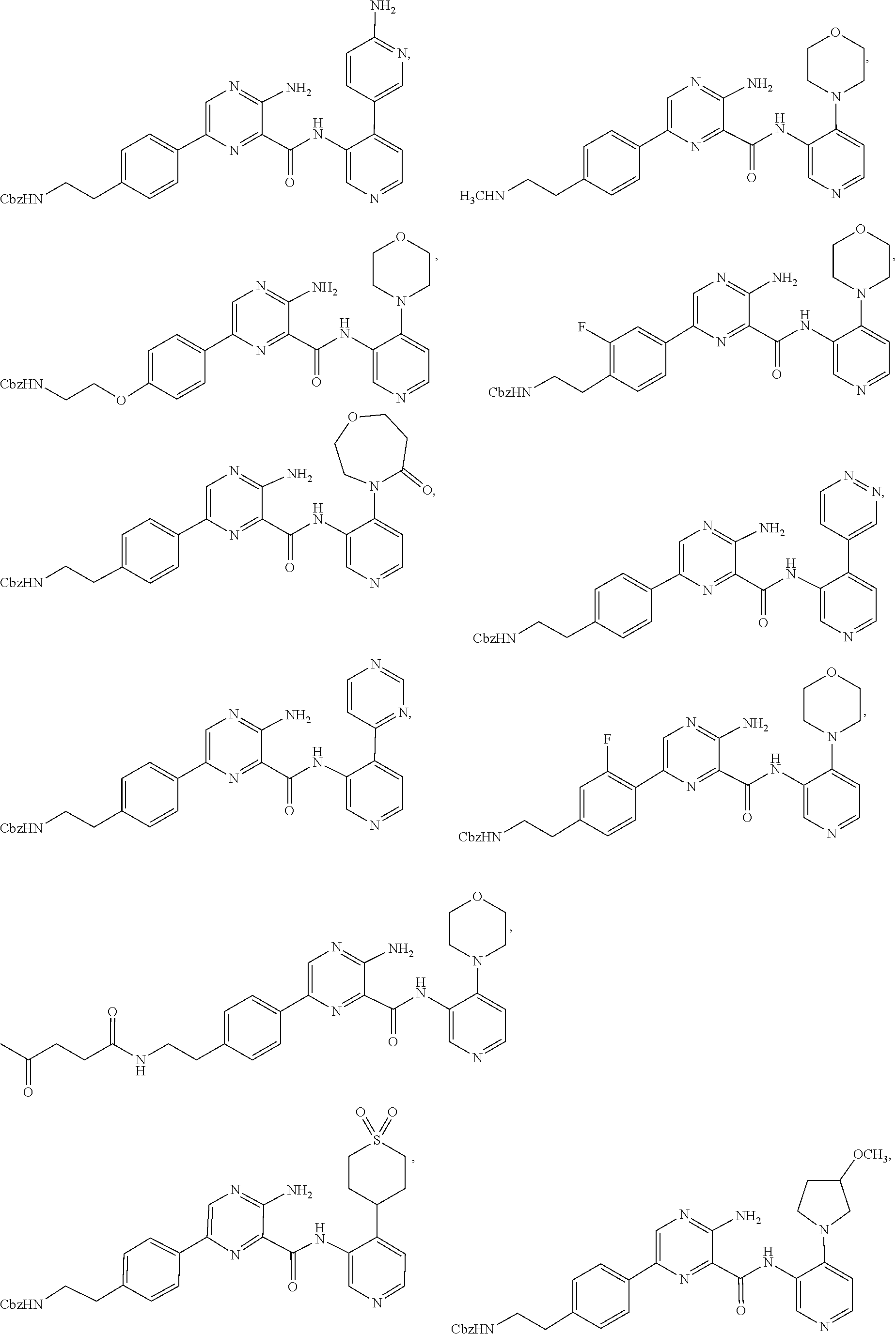

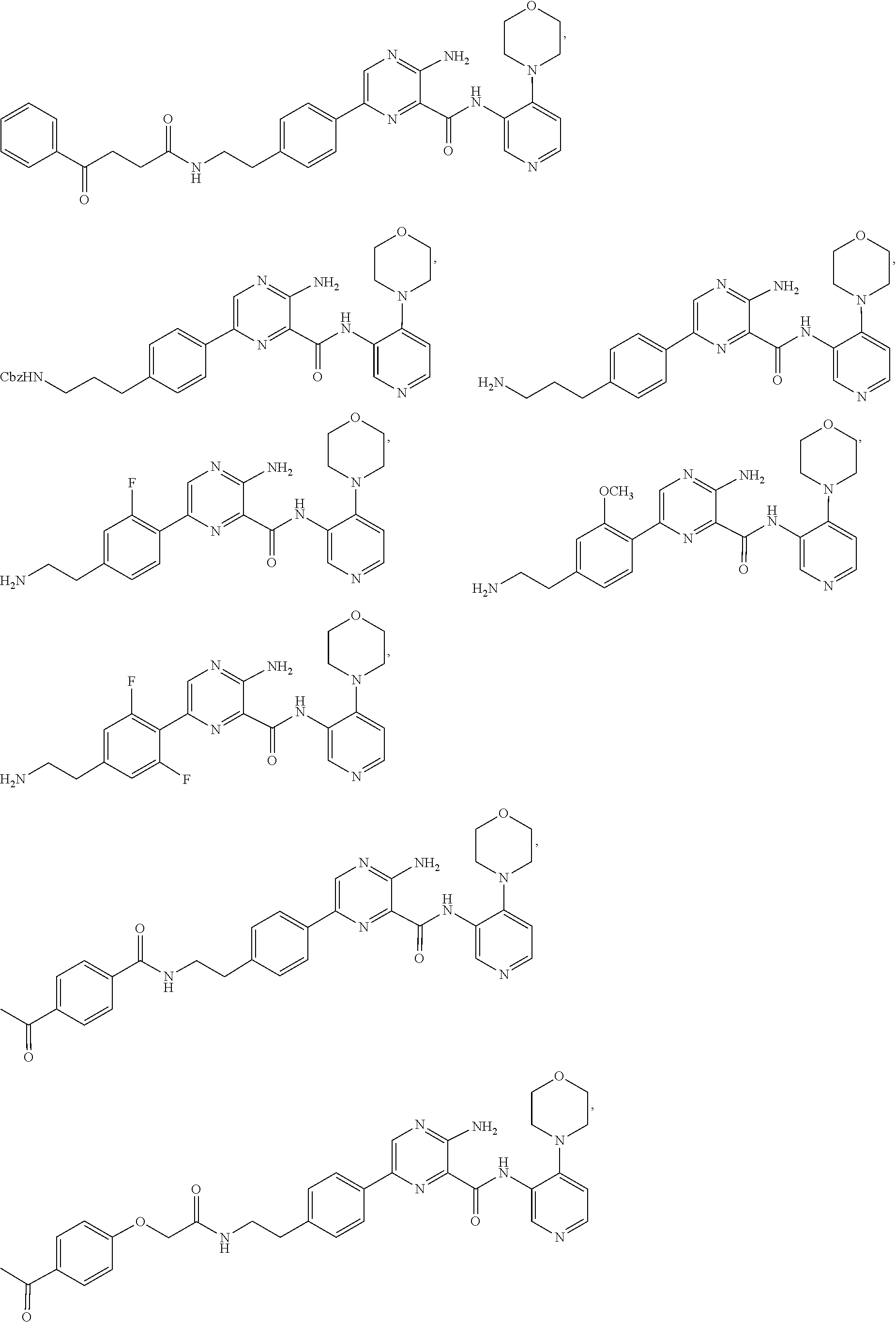

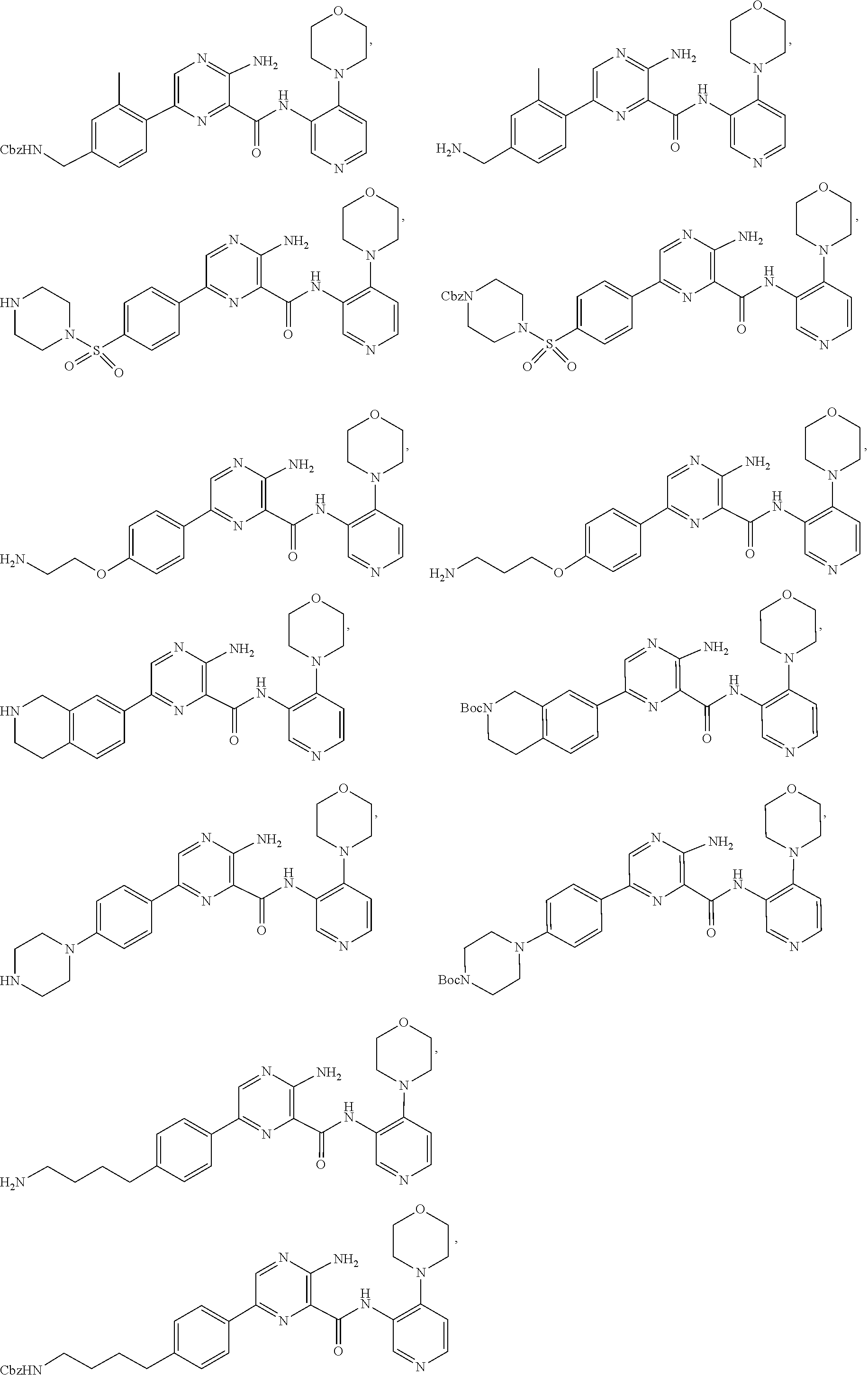

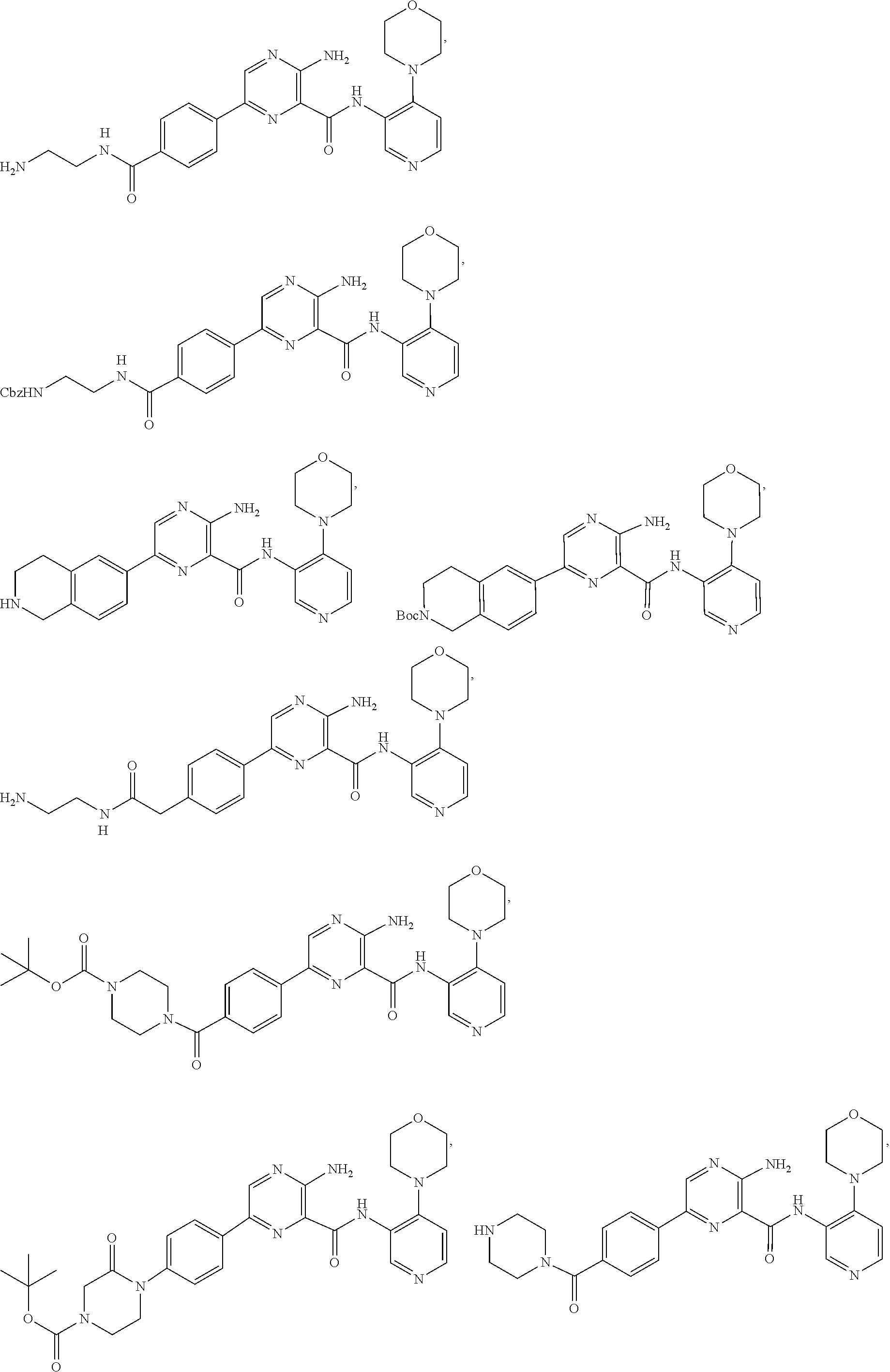

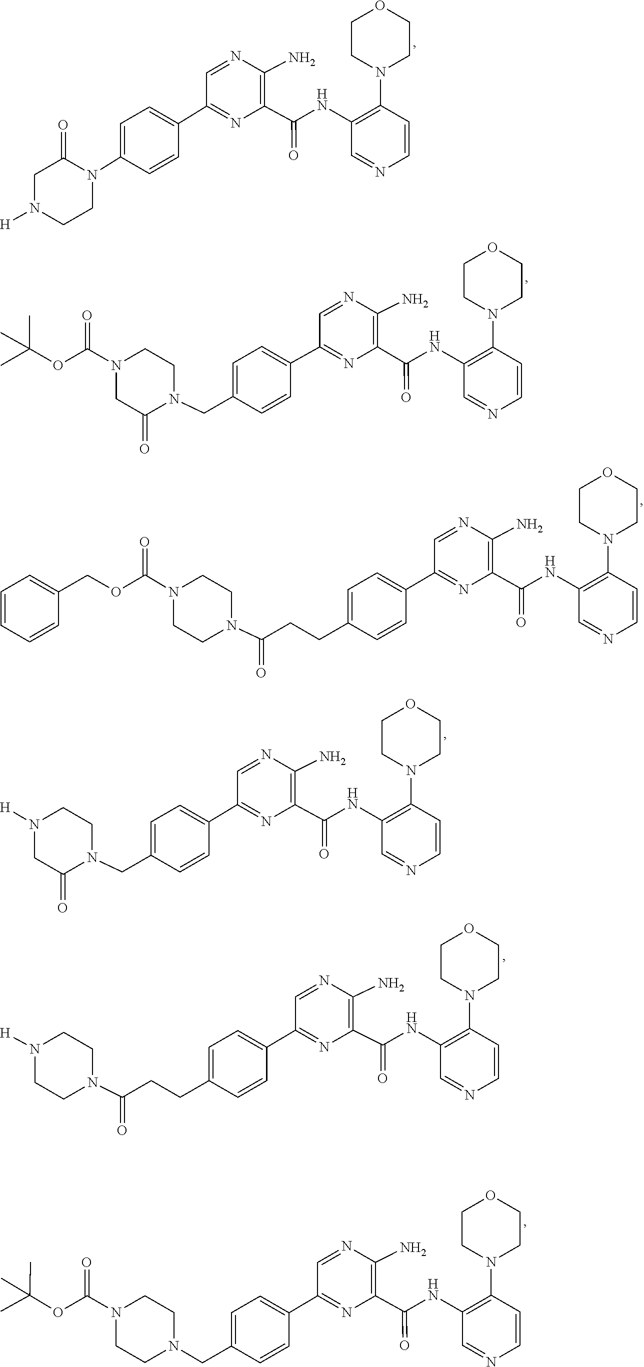 [SMILES: Nc1ncc(-c2ccc(N3CCNCC3=O)cc2)nc1C(=O)Nc1cnccc1N1CCOCC1 ; CC(C)(C)OC(=O)N1CCN(Cc2ccc(-c3cnc(N)c(C(=O)Nc4cnccc4N4CCOCC4)n3)cc2)C(=O)C1 ; Nc1ncc(-c2ccc(CCC(=O)N3CCN(C(=O)OCc4ccccc4)CC3)cc2)nc1C(=O)Nc1cnccc1N1CCOCC1 ; Nc1ncc(-c2ccc(CN3CCNCC3=O)cc2)nc1C(=O)Nc1cnccc1N1CCOCC1 ; Nc1ncc(-c2ccc(CCC(=O)N3CCNCC3)cc2)nc1C(=O)Nc1cnccc1N1CCOCC1 ; CC(C)(C)OC(=O)N1CCN(Cc2ccc(-c3cnc(N)c(C(=O)Nc4cnccc4N4CCOCC4)n3)cc2)CC1]

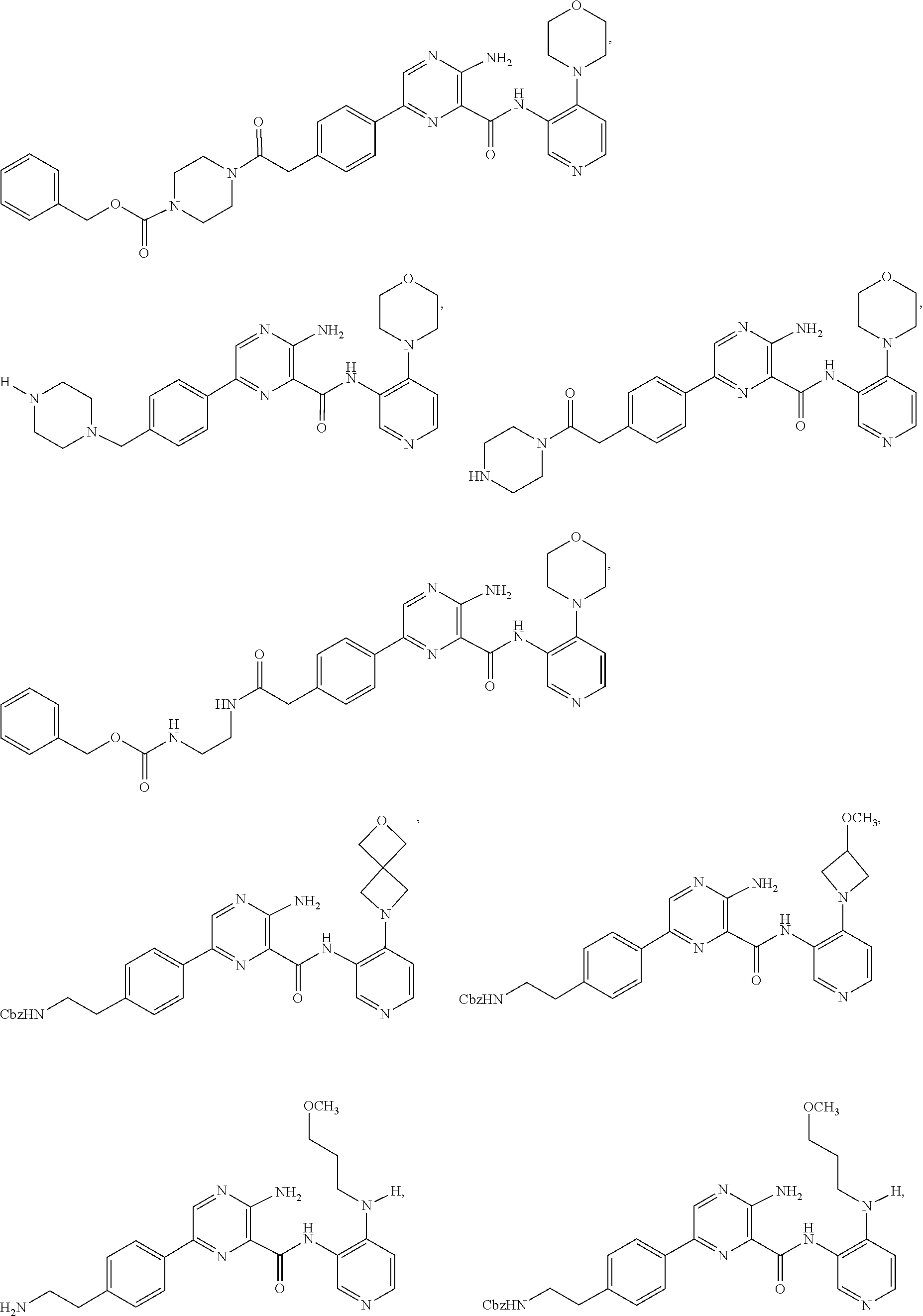

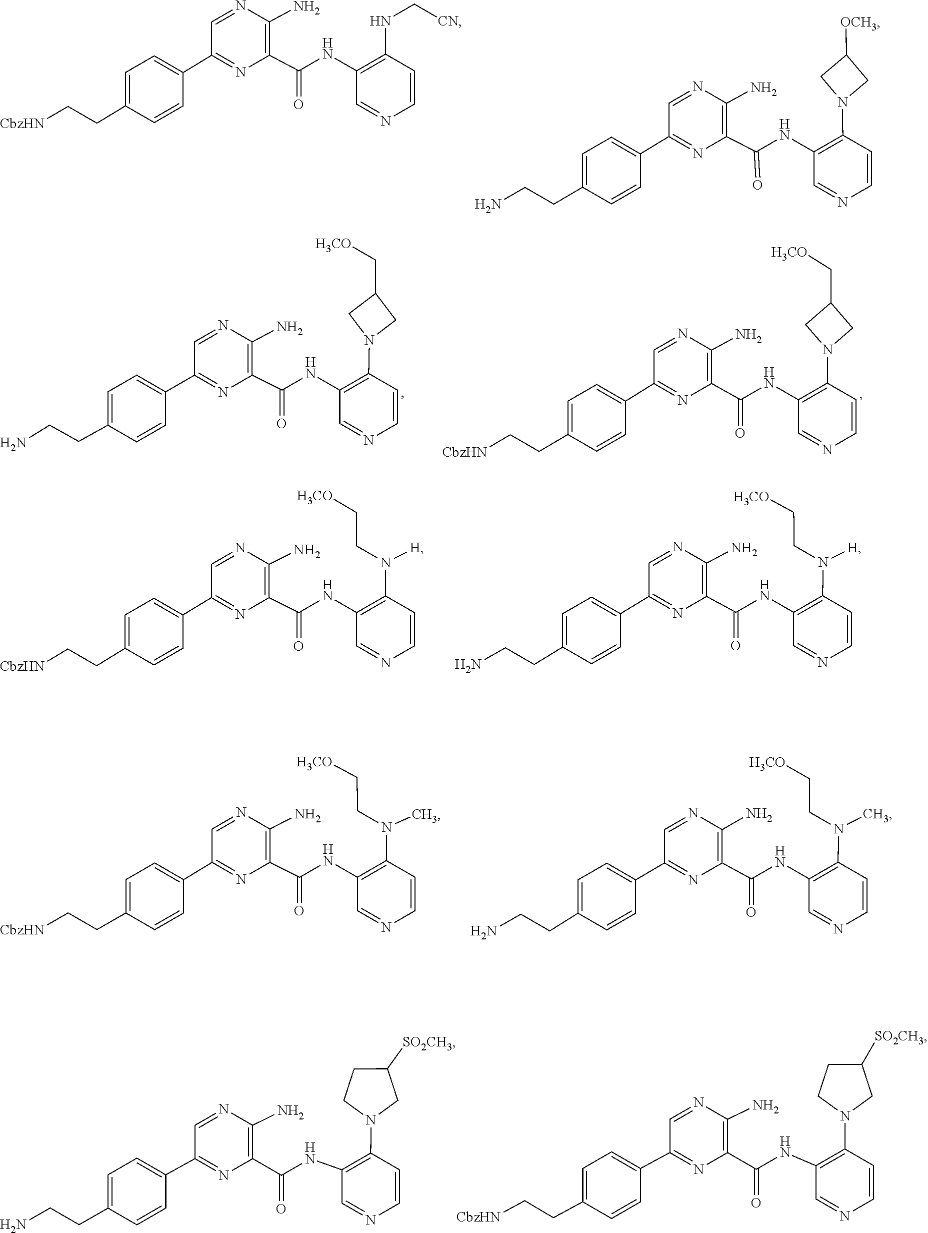

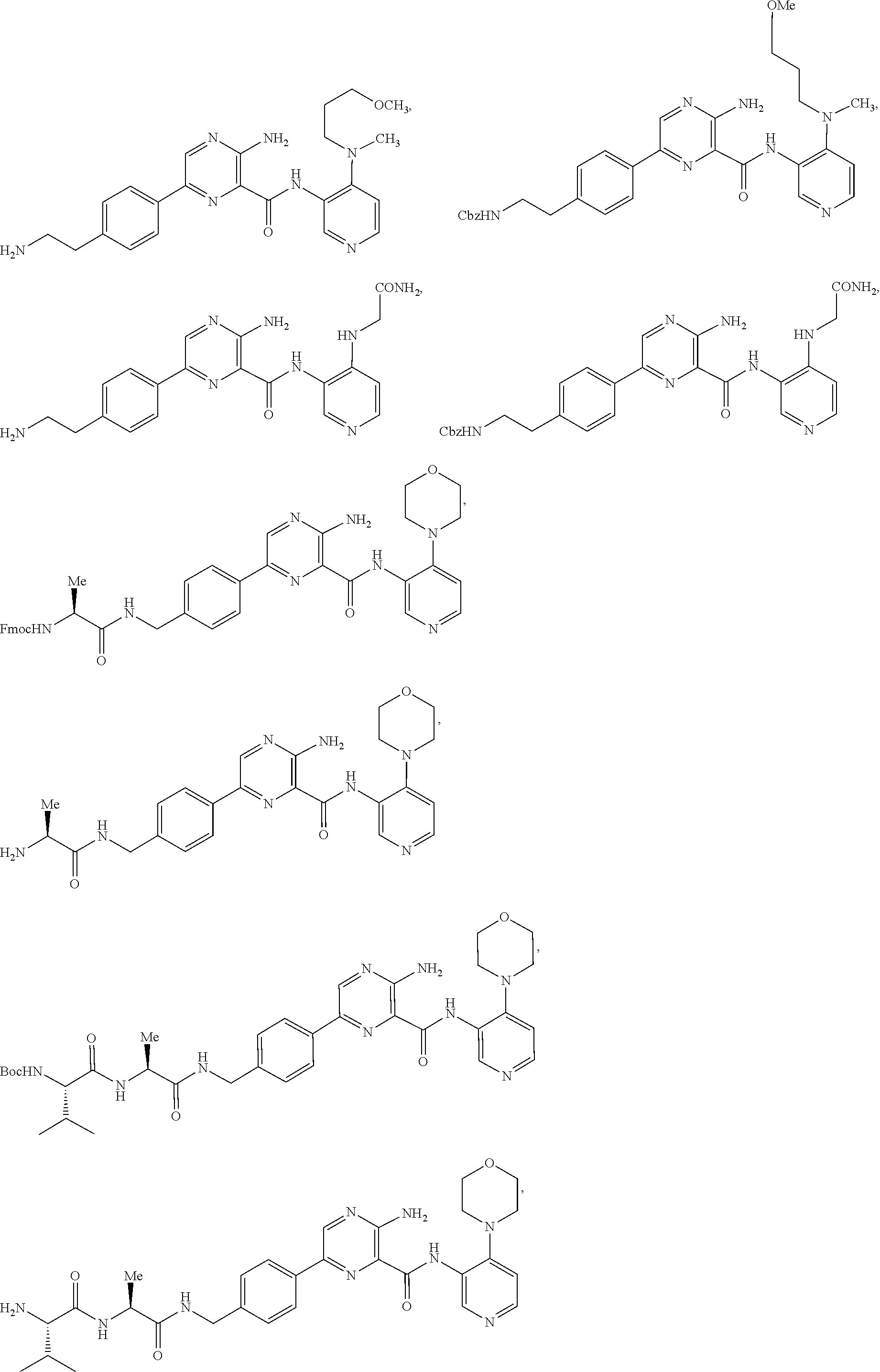

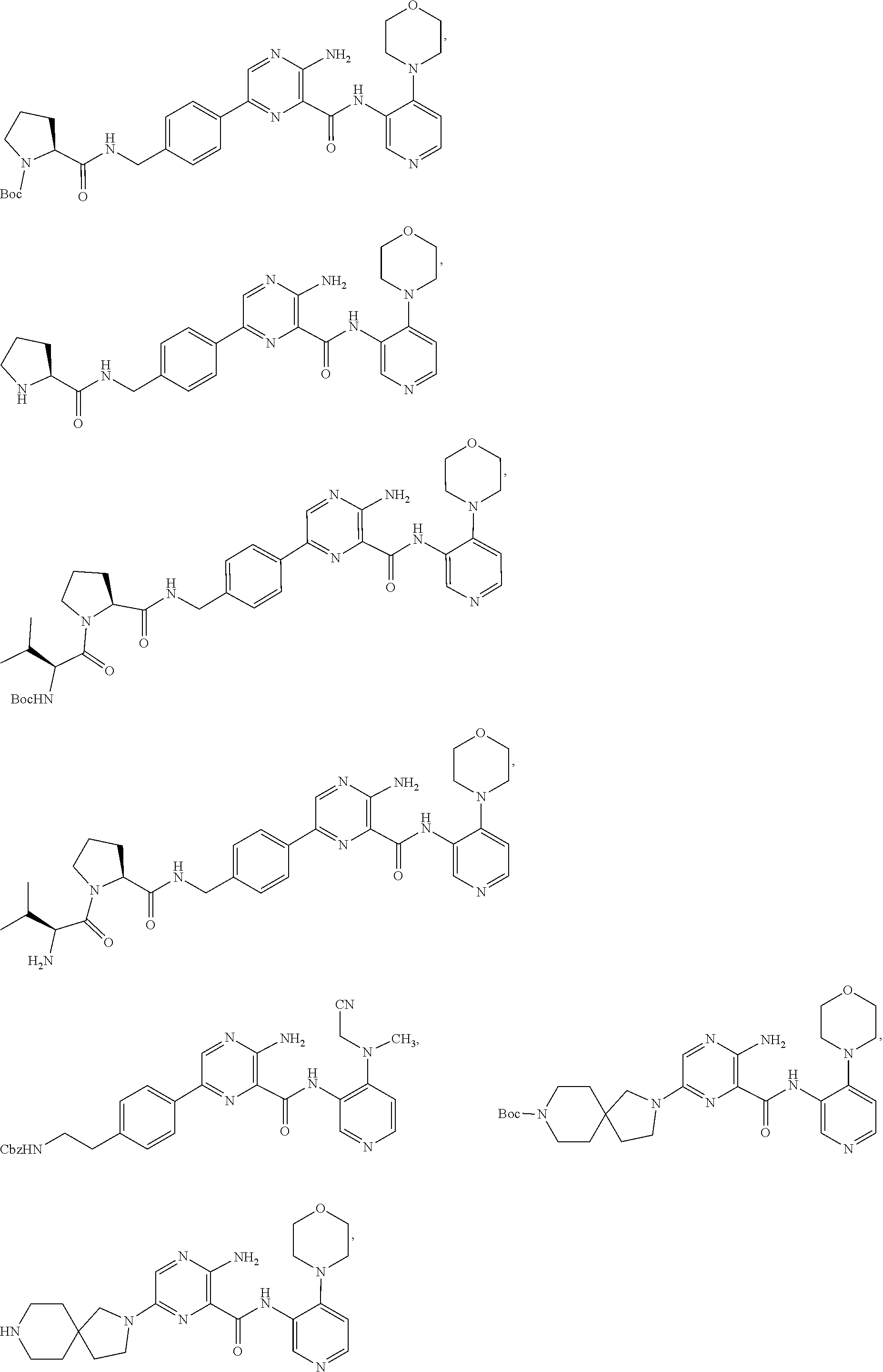

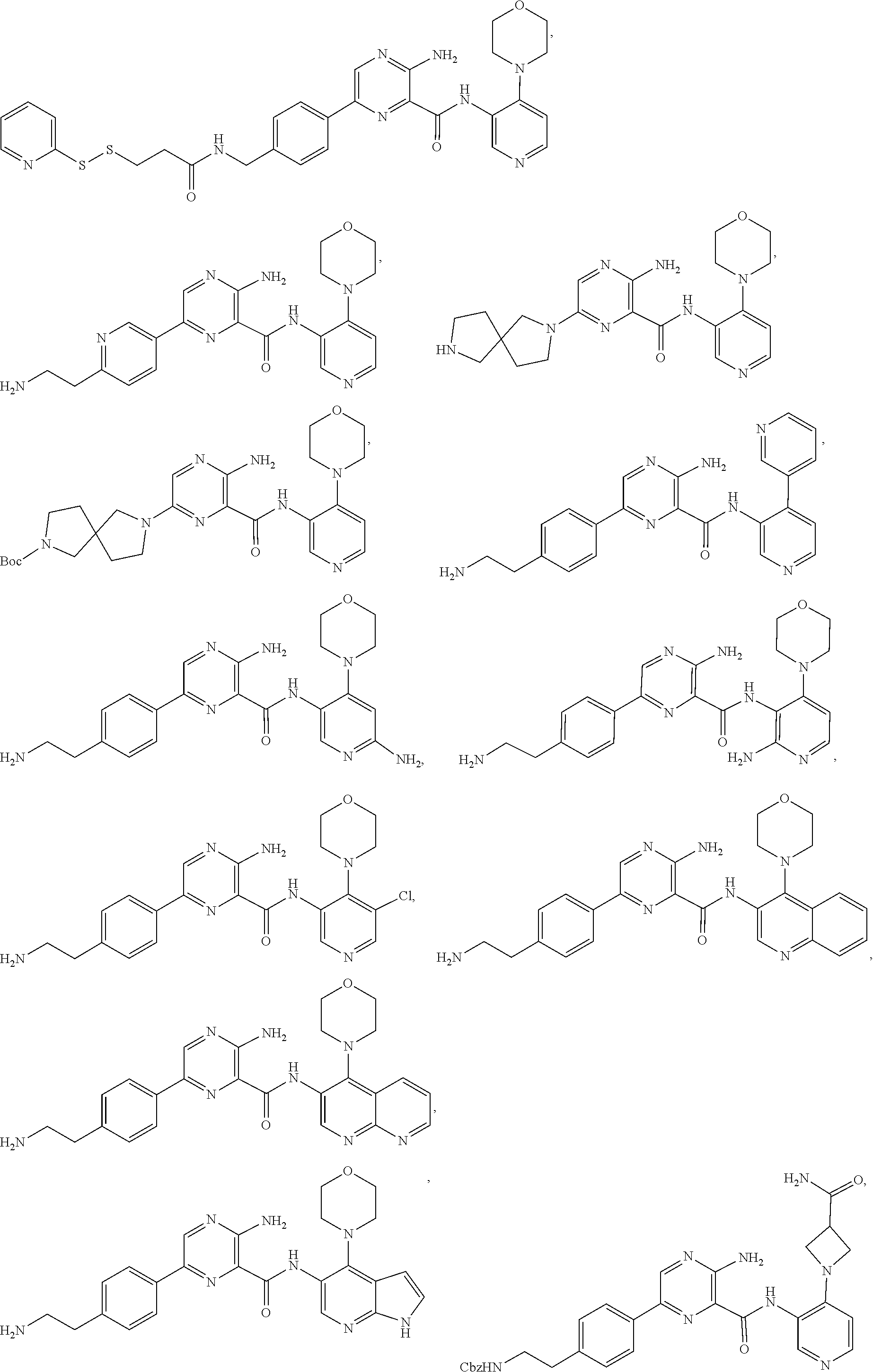

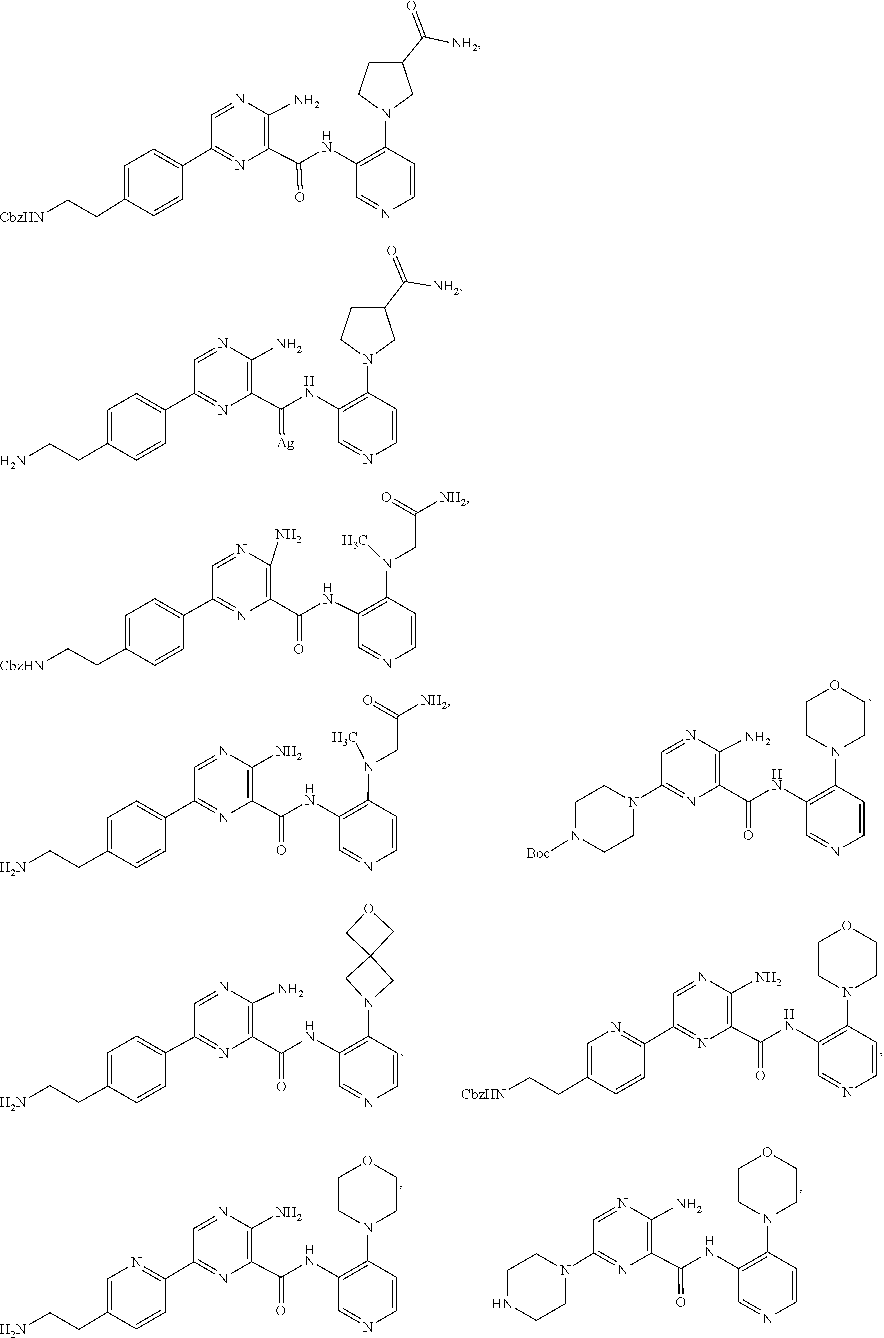

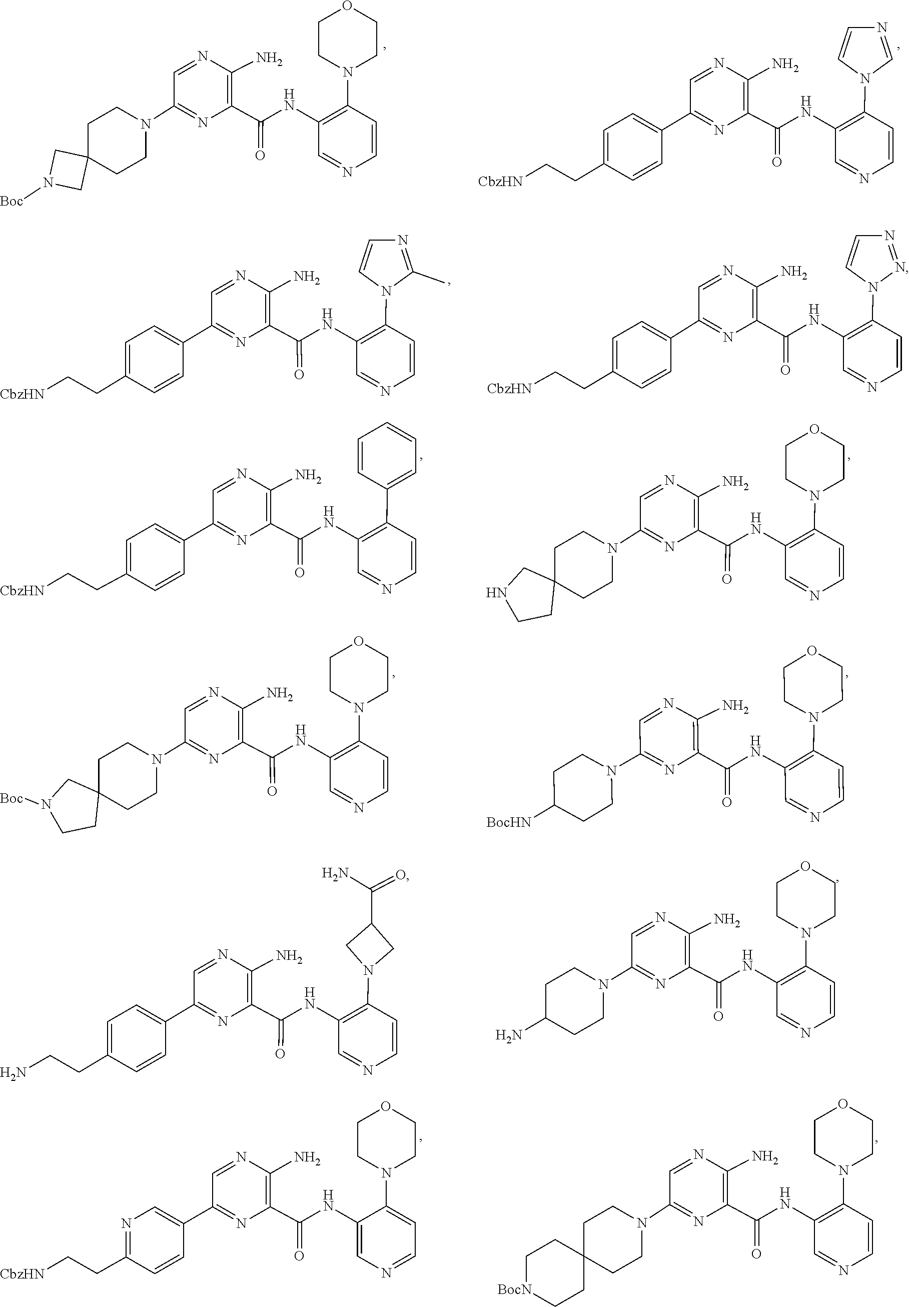

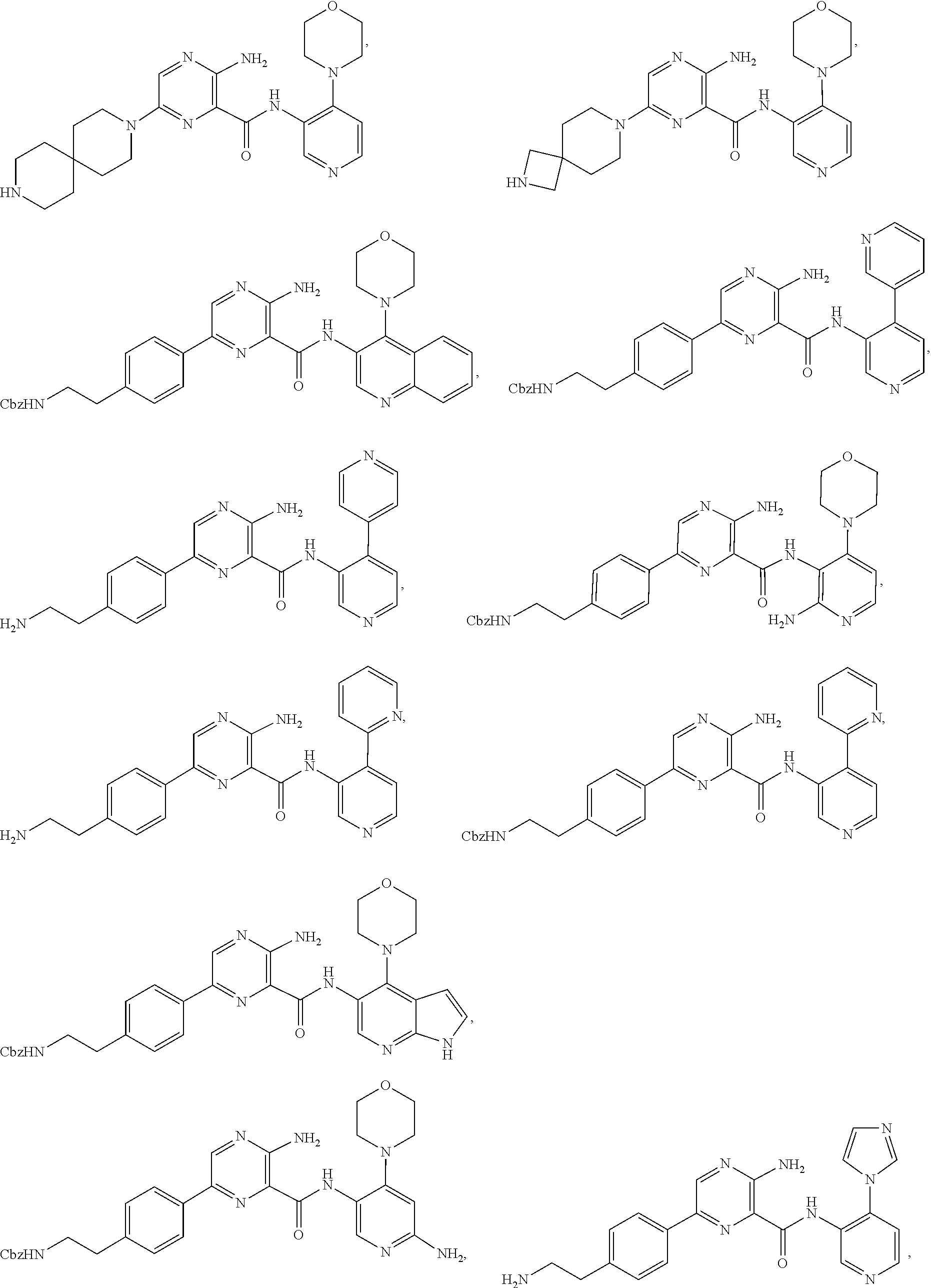

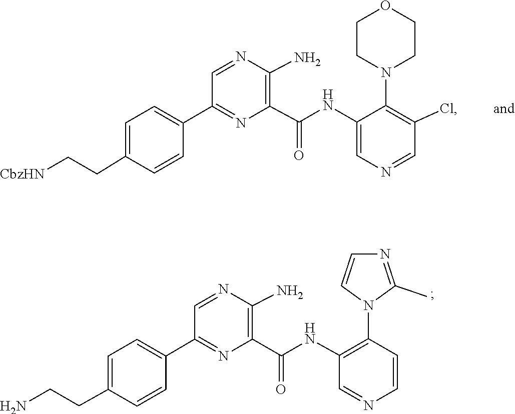 [SMILES: Nc1ncc(-c2ccc(CCNC(=O)OCc3ccccc3)cc2)nc1C(=O)Nc1cncc(Cl)c1N1CCOCC1 ; "and" ; Cc1nccn1-c1ccncc1NC(=O)c1nc(-c2ccc(CCN)cc2)cnc1N]

or a salt of any one thereof.

Also included in the present invention are compounds represented by Formula (I):

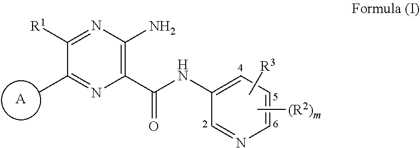

or a pharmaceutically acceptable salt thereof, wherein:
Ring A is unsubstituted or substituted cycloalkyl, unsubstituted or substituted heterocycloalkyl, unsubstituted or substituted aryl, or unsubstituted or substituted heteroaryl, wherein when Ring A is substituted, substituents on Ring A are independently selected at each occurrence from R⁴;
   each R⁴ is selected from R^{L} and R²⁰, or two R⁴ on adjacent atoms are taken together with the atoms to which they are attached to form an unsubstituted or substituted monocyclic carbocycle or unsubstituted or substituted monocyclic heterocycle;
   R^{L} is

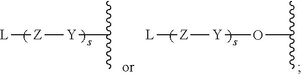

each Y is independently unsubstituted or substituted C₁-C₆alkylene; wherein when Y is substituted, substituents on Y are independently selected at each occurrence from R⁵;
each R⁵ is selected from R²⁰, or two R⁵ on adjacent atoms are taken together with the atoms to which they are attached to form an unsubstituted or substituted monocyclic carbocycle, or unsubstituted or substituted monocyclic heterocycle;
each Z is independently —NR⁶S(═O)₂—, —S(═O)₂NR⁶—, —OC(═O)—, —C(═O)O—, —C(═O)NR⁶—, or —NR⁶C(═O)—; wherein each R⁶ is independently selected from hydrogen, unsubstituted or substituted C₁-C₆alkyl, unsubstituted or substituted carbocycle, and unsubstituted or substituted heterocycle, or an R⁵ and an R⁶ on adjacent atoms are taken together with the atoms to which they are attached to form an unsubstituted or substituted monocyclic heterocycle;
L is unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; wherein when L is substituted, substituents on L are independently selected at each occurrence from R⁷;
   each R⁷ is selected from —SSR⁵⁰ and R²⁰;
s is 1-10;
R¹ is selected from hydrogen and R²⁰;
each R² is independently selected from R²⁰, or two R² on adjacent atoms are taken together with the atoms to which they are attached to form an unsubstituted or substituted monocyclic carbocycle or unsubstituted or substituted monocyclic heterocycle; m is 0-3;
R³ is selected from (i), (ii), (iii), and (iv):
   (v) unsubstituted or substituted aryl, or unsubstituted or substituted heteroaryl; wherein when R³ is substituted, substituents on R³ are independently selected at each occurrence from R¹⁰;
   (vi) unsubstituted or substituted cycloalkyl, or unsubstituted or substituted heterocycloalkyl; wherein when R³ is substituted, substituents on R³ are independently selected at each occurrence from R¹¹;
   (vii) unsubstituted or substituted polycyclic heterocycloalkyl, unsubstituted or substituted 3- to 5-membered monocyclic heterocycloalkyl, unsubstituted or substituted 6- to 8-membered monocyclic heterocycloalkyl comprising 1 or 2 N atoms and 1 or 2 other heteroatoms selected from O or S; wherein when R³ is substituted, substituents on R³ are independently selected at each occurrence from R¹¹; and

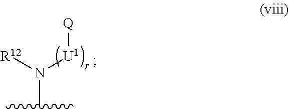

wherein when R³ is at the 2-, 5-, or 6-position of the pyridine, R³ is selected from (i), (ii), and (iv), and when R³ is at the 4-position of the pyridine, R³ is selected from (i), (iii), and (iv); and
   each R¹⁰ is selected from R²⁰, or two R¹⁰ on adjacent atoms are taken together with the atoms to which they are attached to form an unsubstituted or substituted monocyclic carbocycle or unsubstituted or substituted monocyclic heterocycle;
   each R¹¹ is selected from ═O, ═S, and R²⁰; R¹² is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   Q is —OR¹³, —NR¹³R¹³, —SR¹³, —CN, —C(═O)R¹⁴, —C(═O)NR¹³R¹³, —S(═O)R¹⁴, or —S(═O)₂R¹⁴, or —S(═O)₂NR¹³R¹³.
      R¹³ is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
      R¹⁴ is unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
   each U¹ is —(CR¹⁵R¹⁶)—, wherein each R¹⁵ and R¹⁶ are independently selected from hydrogen and R²⁰;
   r is 1-5;
each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
each R⁵⁰ is independently selected from unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
each R⁵¹ is independently selected from hydrogen, unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
or two R⁵¹ on the same N atom are taken together with the N atom to which they are attached to form an unsubstituted or substituted N-containing heterocycle;
   wherein when any of R², R⁴, R⁵, R⁶, R¹⁰, R¹², R¹³, R¹⁴, R²⁰, R⁵⁰, and R⁵¹ are substituted, substituents on the R², R⁴, R⁵, R⁶, R¹⁰, R¹², R¹³, R¹⁴, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted or substituted C₁-C₆alkyl, C₁-C₆haloalkyl, unsubstituted or substituted monocyclic carbocycle, unsubstituted or substituted monocyclic heterocycle, or two substituents on the same carbon atom are taken together to form a C═O or C═S and wherein substituents on said C₁-C₆alkyl are independently selected from R⁵⁴ and substituents on said carbocycle and heterocycle are independently selected from R⁵⁵each R⁵² is independently selected from hydrogen, C₁-C₆alkyl, C₃-C₆cycloalkyl, phenyl, benzyl, 5-membered heteroaryl, and 6-membered heteroaryl;
   or two R⁵² groups are taken together with the N atom to which they are attached to form a N-containing heterocycle; and
   each R⁵³ is independently selected from C₁-C₆alkyl, C₃-C₆cycloalkyl, phenyl, benzyl, 5-membered heteroaryl, and 6-membered heteroaryl.
   each R⁵⁴ is independently selected from —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², and phenyl;
   each R⁵⁵ is independently selected from —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², and unsubstituted or substituted C₁-C₆alkyl wherein substituents on said C₁-C₆alkyl are independently selected from R⁵⁴.

Compounds of Formula (I) include those wherein any of R², R⁴, R⁵, R⁶, R¹⁰, R¹², R¹³, R¹⁴, R²⁰, R⁵⁰, and R⁵¹ are substituted, substituents on the R², R⁴, R⁵, R⁶, R¹⁰, R¹², R¹³, R¹⁴, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted or substituted C₁-C₆alkyl, C₁-C₆haloalkyl, monocyclic carbocycle, monocyclic heterocycle, or two substituents on the same carbon atom are taken together to form a C═O or C═S; and wherein substituents on said C₁-C₆alkyl are independently selected from R⁵⁴.

Compounds of Formula (I) include those wherein when Ring A is aryl, ring A is substituted and substituents on Ring A are independently selected at each occurrence from R⁴; and each R⁴ is selected from R²⁰ and

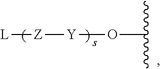

or two R⁴ on adjacent atoms are taken together with the atoms to which they are attached to form an unsubstituted or substituted monocyclic carbocycle or unsubstituted or substituted monocyclic heterocycle; and each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle, or substituted C₁-C₆alkyl; with the proviso that when C₁-C₆alkyl is substituted with —C(═O)NR⁵²R⁵², —NR⁵²R⁵², —NR⁵²C(═O)R⁵³, or —NR⁵²C(═O)OR⁵², said C₁-C₆alkyl is further substituted with at least one of —OR⁵², —CO₂R⁵², —(C₁-C₆alkyl)-OR⁵², or —(C₁-C₆alkyl)-CO₂R⁵².

Compounds of Formula (I) include those wherein when Ring A is aryl, ring A is substituted and substituents on Ring A are independently selected at each occurrence from R⁴; and each R⁴ is selected from R²⁰ and

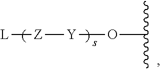

or two R⁴ on adjacent atoms are taken together with the atoms to which they are attached to form an unsubstituted or substituted monocyclic carbocycle or unsubstituted or substituted monocyclic heterocycle; and each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle, or substituted C₁-C₆alkyl; with the proviso that when C₁-C₆alkyl is substituted with —C(═O)NR⁵²R⁵², —NR⁵²R⁵², —NR⁵²C(═O)R⁵³, or —NR⁵²C(═O)OR⁵², said C₁-C₆alkyl is further substituted with at least one of —OR⁵², —CO₂R⁵², —(C₁-C₆alkyl)-OR⁵², or —(C₁-C₆alkyl)-CO₂R⁵². Also included are those compounds wherein when R²⁰ is C₁-C₆alkyl substituted with —OR⁵², said C₁-C₆alkyl is further substituted with—at least one of C(═O)NR⁵²R⁵², —NR⁵²R⁵², —NR⁵²C(═O)R⁵³, or —NR⁵²C(═O)OR⁵². Also included are those compounds wherein R²⁰ is C₁-C₆alkyl substituted with —OR⁵², said C₁-C₆alkyl is further substituted with —NR⁵²R⁵². Also included are those compounds wherein R²⁰ is C₁-C₆alkyl substituted with —NR⁵²R⁵² and at least one of —OR⁵², —CO₂R⁵², —(C₁-C₆alkyl)-OR⁵², or (C₁-C₆alkyl)-CO₂R⁵².

Exemplary compounds of the present invention include those set forth in Table 14 and salts thereof (including pharmaceutically acceptable salts thereof).

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

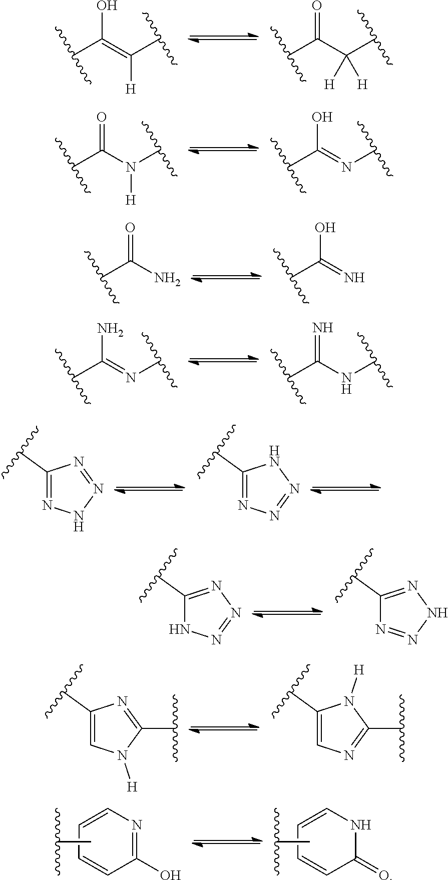

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In particular embodiments, the parent compound with the acidic moiety is retained within a cell for a longer duration than the same compound without the acidic moiety.

The parent compound, with an acidic moiety, may be retained within the cell, i.e., drug residence, for 10% or longer, such as 15% or longer, such as 20% or longer, such as 25% or longer, such as 30% or longer, such as 35% or longer, such as 40% or longer, such as 45% or longer, such as 50% or longer, such as 55% or longer, such as 60% or longer, such as 65% or longer, such as 70% or longer, such as 75% or longer, such as 80% or longer, such as 85% or longer, or even 90% or longer relative to the same compound without an acidic moiety.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J Pharmaceutics, 37, 87 (1987); J. Larsen et al., Int. J Pharmaceutics, 47, 103 (1988); Sinkula et al., J Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques.

Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers

The compounds and salts described herein may be bound to a linker, e.g., a peptide linker or a non-cleavable linker. In certain embodiments, the linker is also bound to an antibody construct and may be referred to as an antibody conjugate or conjugate. Linkers of the conjugates may not affect the binding of active portions of a conjugate, e.g., the antigen binding domains, Fc domains, target binding domains, antibodies, amino-pyrazinecarboxamide compounds or the like, to an antigen. A conjugate can comprise multiple linkers, each having one or more compounds attached. These linkers can be the same linkers or different linkers.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker may comprise a valine-citrulline linker or a valine-alanine peptide. A valine-citrulline- or valine-alanine-containing linker can contain a pentafluorophenyl group. A valine-citrulline- or valine-alanine-containing linker can contain a maleimide or succinimide group. A valine-citrulline- or valine-alanine-containing linker can contain a para aminobenzoic acid (PABA) group. A valine-citrulline- or valine-alanine-containing linker can contain a PABA group and a pentafluorophenyl group. A valine-citrulline- or valine-alanine-containing linker can contain a PABA group and a maleimide or succinimide group.

A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker. A linker can be a linker suitable for attachment to an engineered cysteine (THIOMAB), such as a (maleimidocaproyl)-(valine-citrulline)-(para-aminobenzyloxycarbonyl)-linker.

A linker can also comprise alkylene, alkenylene, alkynylene, polyether, polyester, polyamide group(s) and also, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG (SEQ ID NO:49) recognition motif to an N-terminal GGG motif to regenerate a native amide bond. The linker created can therefore link a moiety attached to the LPXTG (SEQ ID NO:49) recognition motif with a moiety attached to the N-terminal GGG motif.

In the conjugates, a compound or salt of any one of Formulas (I-A), (I-B), (I-C), (I-D), (I-E), (II-A), (II-B), (II-C), and (II-D) and Table 14 is linked to the antibody by way of a linker(s), also referred to herein as L³. L³, as used herein, may be selected from any of the linker moieties discussed herein. The linker linking the compound or salt to the antibody construct of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a single site on the antibody construct, or monovalent such that covalently they link a single compound or salt to a single site on the antibody construct.

Linkers of the disclosure (L³) may have from about 10 to about 500 atoms in a linker, such as from about 10 to about 400 atoms, such as about 10 to about 300 atoms in a linker. In certain embodiments, linkers of the disclosure have from about 30 to about 400 atoms, such as from about 30 to about 300 atoms in the linker.

As will be appreciated by skilled artisans, the linkers may link a compound or salt of any one of Formulas (I-A), (I-B), (I-C), (I-D), (I-E), (II-A), (II-B), (II-C), and (II-D) and Table 14 to the antibody construct by a covalent linkages between the linker and the antibody construct and compound. As used herein, the expression “linker” is intended to include (i) unconjugated forms of the linker that include a functional group capable of covalently linking the linker to an amino-pyrazinecarboxamide compound and a functional group capable of covalently linking the linker to an antibody construct; (ii) partially conjugated forms of the linker that include a functional group capable of covalently linking the linker to an antibody construct and that is covalently linked to a compound(s) or salt(s) of any one of Formulas (I-A), (I-B), (I-C), (I-D), (I-E), (II-A), (II-B), (II-C), and (II-D) and Table 14, or vice versa; and (iii) fully conjugated forms of the linker that is covalently linked to both a compound or salt of any one of Formulas (I-A), (I-B), (I-C), (I-D), (I-E), (II-A), (II-B), (II-C), and (II-D) and Table 14 and an antibody construct. One embodiment pertains to a conjugate formed by contacting an antibody construct that binds a cell surface receptor or tumor associated antigen expressed on a tumor cell with a linker-compound described herein under conditions in which the linker-compound covalently links to the antibody construct. One embodiment pertains to a method of making a conjugate formed by contacting a linker-compound under conditions in which the linker-compound covalently links to the antibody construct.

In certain embodiments, any one of the compounds or salts described in the section entitled “Compounds” is covalently bound to a linker (L³). The linker may be covalently bound to any position, valence permitting. The linker may comprise a reactive moiety, e.g., an electrophile that can react to form a covalent bond with a moiety of an antibody construct such as, for example, a lysine, serine, threonine, cysteine, tyrosine, aspartic acid, glutamine, a non-natural amino acid residue, or glutamic acid residue. In some embodiments, a compound or salt of a compound in the section entitled “Compounds” herein is covalently bound through the linker to an antibody construct.

Exemplary polyvalent linkers that may be used to link many amino-pyrazinecarboxamide compounds to an antibody construct are described. For example, Fleximer® linker technology has the potential to enable high-DAR conjugates with good physicochemical properties. As shown below, the Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties. This methodology could be utilized with amino-pyrazinecarboxamide compound as shown in the Scheme below.

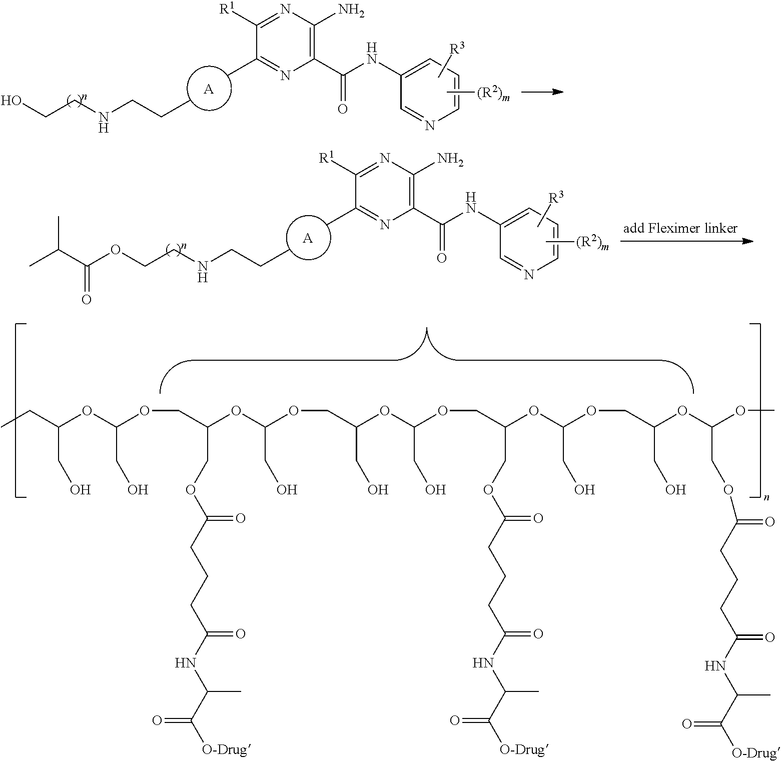

To utilize the Fleximer® linker technology depicted in the scheme above, an aliphatic alcohol can be present or introduced into the amino-pyrazinecarboxamide compound. The alcohol moiety is then conjugated to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates are described below, in addition to those previously described.

Sulfamide linkers may be used to link many amino-pyrazinecarboxamide compounds to an antibody construct. Sulfamide linkers are as described herein and e.g., U.S. Patent Publication Number 2019/0038765, the linkers of which are incorporated by reference herein

Cleavable linkers can be cleavable in vitro and in vivo. Cleavable linkers can include chemically or enzymatically unstable or degradable linkages. Cleavable linkers can rely on processes inside the cell to liberate an amino-pyrazinecarboxamide compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate release of an amino-pyrazinecarboxamide compound for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the amino-pyrazinecarboxamide compound once the antibody conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

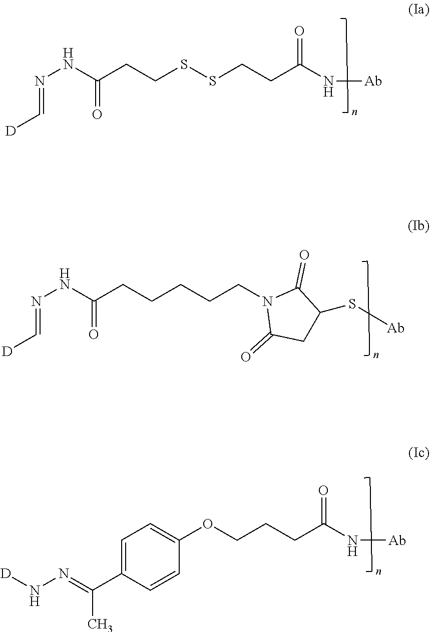

wherein D is a compound or salt of any one of Formulas (I-A), (I-B), (I-C), (I-D), (I-E), (II-A), (II-B), (II-C), and (II-D), and Ab is an antibody construct, respectively, and n represents the number of compound-bound linkers (LP) bound to the antibody construct. In certain linkers, such as linker (Ia), the linker can comprise two cleavable groups, a disulfide and a hydrazone moiety. For such linkers, effective release of the unmodified free amino-pyrazinecarboxamide compound can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the amino-pyrazinecarboxamide compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing an amino-pyrazinecarboxamide compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Antibody conjugates containing amino-pyrazinecarboxamide compounds that include exemplary disulfide-containing linkers can include the following structures:

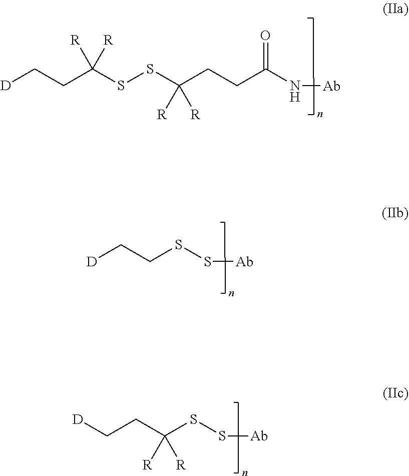

wherein D is a compound or salt of any one of Formulas (I-A), (I-B), (I-C), (I-D), (I-E), (II-A), (II-B), (II-C), and (II-D), and Ab is an antibody construct, respectively, n represents the number of compounds bound to linkers (L³) bound to the antibody construct and R is independently selected at each occurrence from hydrogen or alkyl, for example. Increasing steric hindrance adjacent to the disulfide bond can increase the stability of the linker. Structures such as (IIa) and (IIc) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of an amino-pyrazinecarboxamide compound from an antibody construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly (SEQ ID NO: 235), Ala-Leu-Ala-Leu (SEQ ID NO: 236) or dipeptides such as Val-Cit, Val-Ala, and Phe-Lys. Dipeptides can have lower hydrophobicity compared to longer peptides.

A variety of dipeptide-based cleavable linkers can be used in the antibody constructs to form conjugates of an amino-pyrazinecarboxamide compound described herein.

Enzymatically cleavable linkers can include a self-immolative spacer to spatially separate the amino-pyrazinecarboxamide compound from the site of enzymatic cleavage. The direct attachment of an amino-pyrazinecarboxamide compound to a peptide linker can result in proteolytic release of an amino acid adduct of the amino-pyrazinecarboxamide compound, thereby impairing its activity. The use of a self-immolative spacer can allow for the elimination of the fully active, chemically unmodified amino-pyrazinecarboxamide compound upon amide bond hydrolysis.

One self-immolative spacer can be a bifunctional para-aminobenzyl alcohol group, which can link to the peptide through the amino group, forming an amide bond, while amine containing amino-pyrazinecarboxamide compounds can be attached through carbamate functionalities to the benzylic hydroxyl group of the linker (to give a p-amidobenzylcarbamate, PABC). The resulting pro-amino-pyrazinecarboxamide compound can be activated upon protease-mediated cleavage, leading to a 1,6-elimination reaction releasing the unmodified amino-pyrazinecarboxamide compound, carbon dioxide, and remnants of the linker group. The following scheme depicts the fragmentation of p-amidobenzyl carbamate and release of the amino-pyrazinecarboxamide compound:

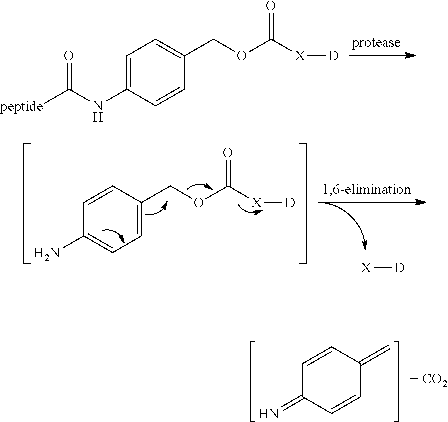

wherein X-D represents the unmodified amino-pyrazinecarboxamide compound.

Heterocyclic variants of this self-immolative group have also been described.

The enzymatically cleavable linker can be a β-glucuronic acid-based linker. Facile release of the amino-pyrazinecarboxamide compound can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of an antibody construct conjugate of an amino-pyrazinecarboxamide compound to undergo aggregation due to the hydrophilic nature of β-glucuronides. In certain embodiments, β-glucuronic acid-based linkers can link an antibody construct to a hydrophobic amino-pyrazinecarboxamide compound. The following scheme depicts the release of an amino-pyrazinecarboxamide compound (D) from an antibody construct conjugate of an amino-pyrazinecarboxamide compound containing a β-glucuronic acid-based linker:

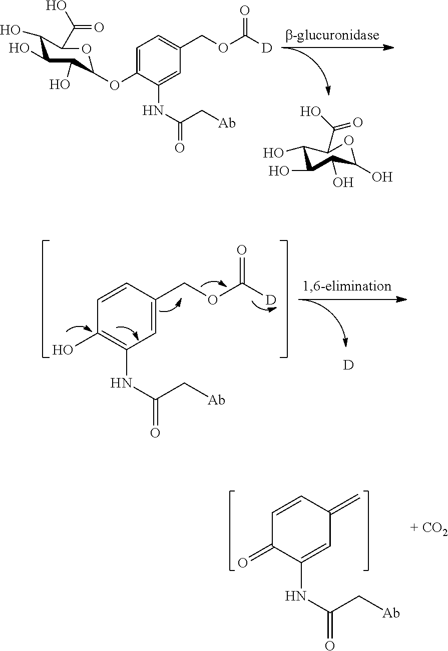

wherein Ab indicates the antibody construct.

A variety of cleavable β-glucuronic acid-based linkers useful for linking drugs such as auristatins, camptothecin and doxorubicin analogues, CBI minor-groove binders, and psymberin to antibodies have been described. These β-glucuronic acid-based linkers may be used in the conjugates. In certain embodiments, the enzymatically cleavable linker is a β-galactoside-based linker. β-Galactoside is present abundantly within lysosomes, while the enzyme activity outside cells is low.

Additionally, amino-pyrazinecarboxamide compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on an amino-pyrazinecarboxamide compound, wherein such ester groups can hydrolyze under physiological conditions to release the amino-pyrazinecarboxamide compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide, for example, a linker comprising structural formula (IIIa), (IIIb), (IIIc), or (IIId):

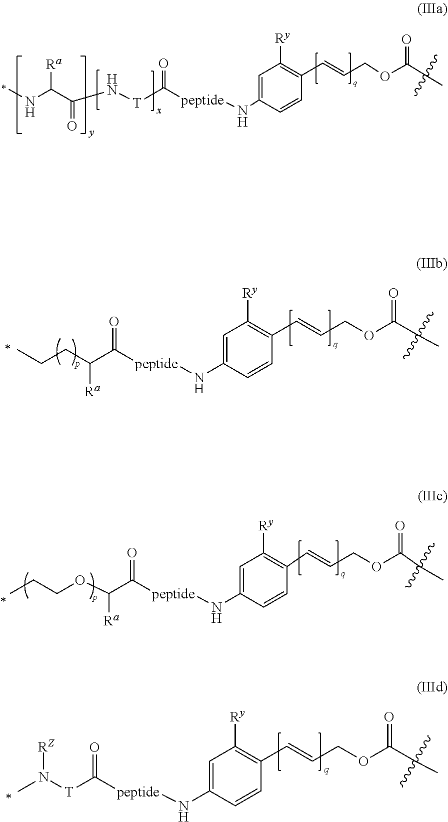

or a salt thereof, wherein: “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or a sugar moiety; G² is SO₃H, CO₂H, or PEG 4-32 moiety; r is 0 or 1; s is 0 or 1; p is an integer ranging from 0 to 5; q is or 1; x is 0 or 1; y is 0 or 1; [symbol] represents the point of attachment of the linker to a compound or salt of any one of Formulas (I-A), (I-B), (I-C), (I-D), (I-E), (II-A), (II-B), (II-C), and (II-D); and * represents the point of attachment to the remainder of the linker.

In certain embodiments, the peptide can be selected from natural amino acids, unnatural amino acids or combinations thereof. In certain embodiments, the peptide can be selected from a tripeptide or a dipeptide. In particular embodiments, the dipeptide can comprise L-amino acids and be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IIIa) are illustrated below (as illustrated, the linkers include a reactive group suitable for covalently linking the linker to an antibody construct):

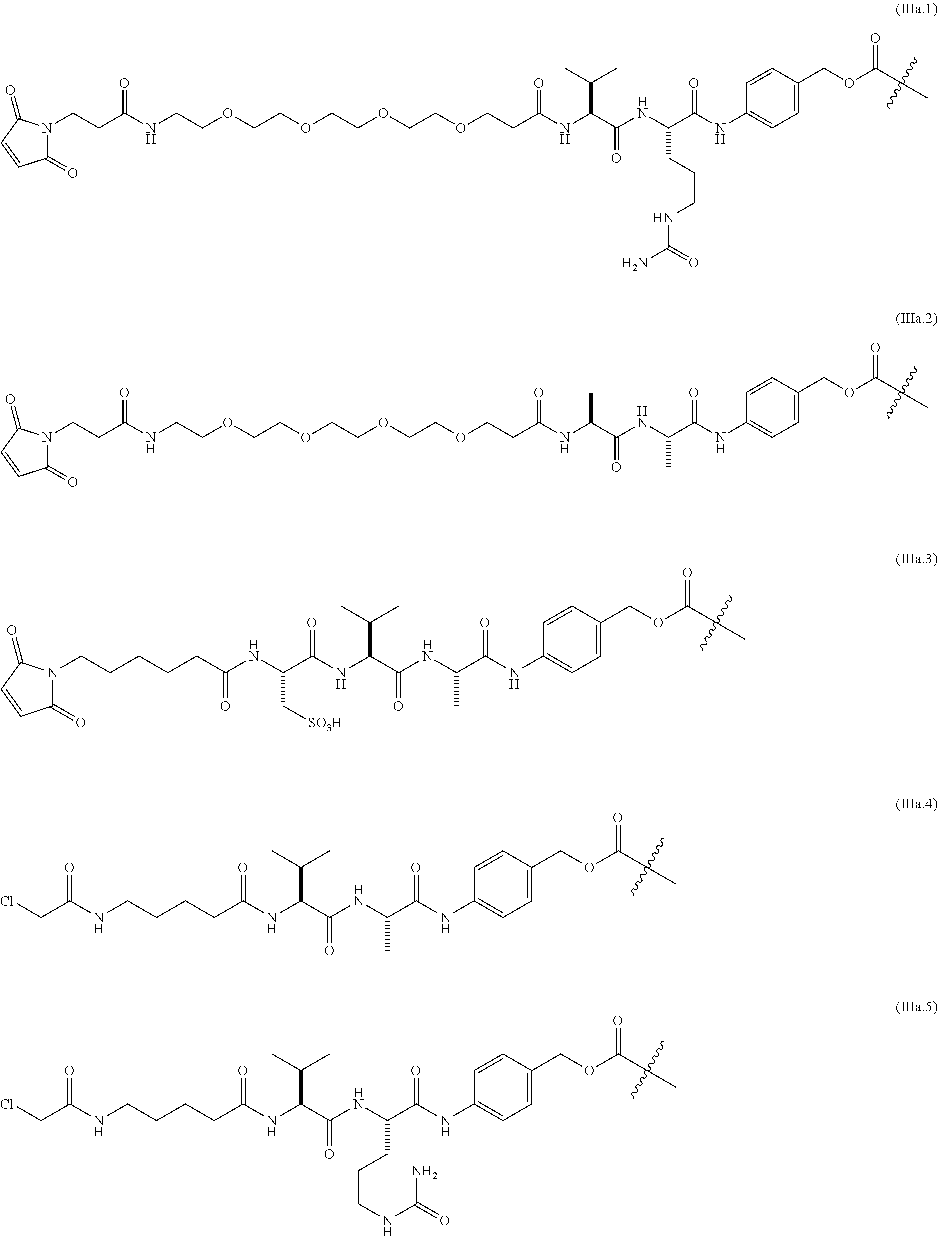 [SMILES: *C(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1 ; *C(=O)OCc1ccc(NC(=O)[C@H](C)NC(=O)[C@H](C)NC(=O)CCOCCOCCOCCOCCNC(=O)CCN2C(=O)C=CC2=O)cc1 ; *C(=O)OCc1ccc(NC(=O)[C@H](C)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCNC(=O)CCl)C(C)C)cc1 ; *C(=O)OCc1ccc(NC(=O)[C@H](CCCNC(N)=O)NC(=O)[C@@H](NC(=O)CCCCNC(=O)CCl)C(C)C)cc1]

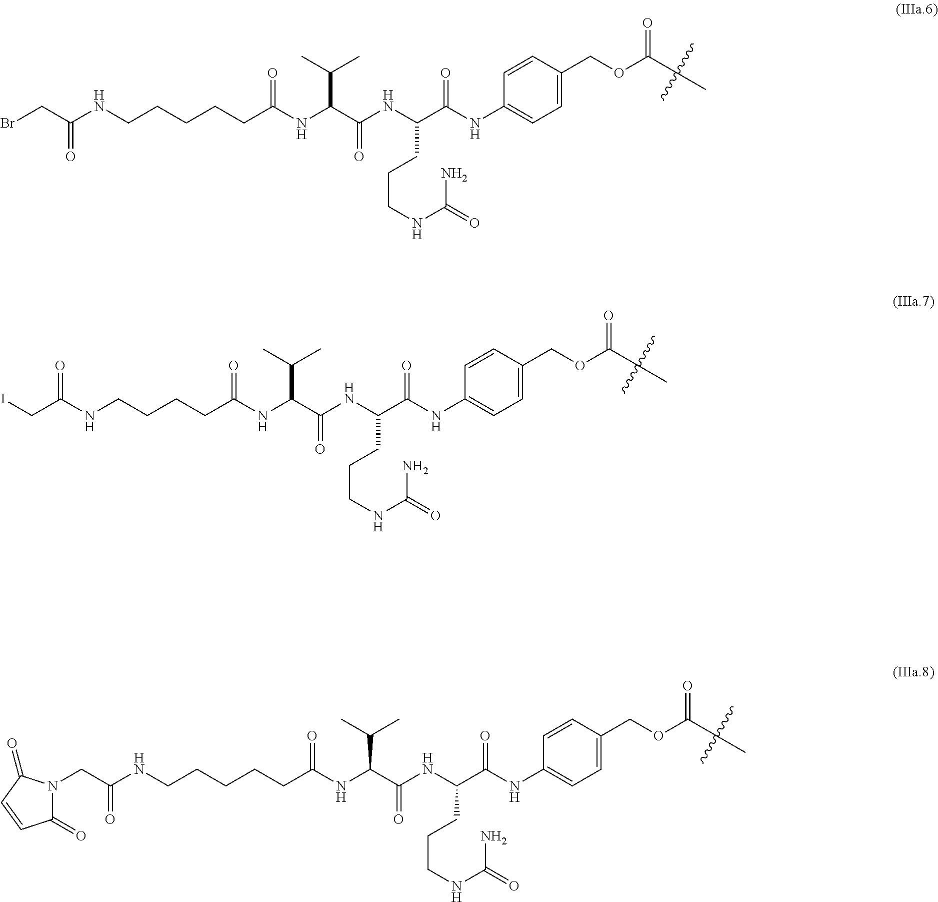 [SMILES: *C(=O)OCc1ccc(NC(=O)[C@H](CCCNC(N)=O)NC(=O)[C@@H](NC(=O)CCCCCNC(=O)CBr)C(C)C)cc1 ; *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

wherein [symbol] indicates an attachment site of a linker (L³) to an amino-pyrazinecarboxamide compound.

Exemplary embodiments of linkers according to structural formula (IIIb), (IIIc), or (IIId) that can be included in the conjugates can include the linkers illustrated below (as illustrated, the linkers include a reactive group suitable for covalently linking the linker to an antibody construct):

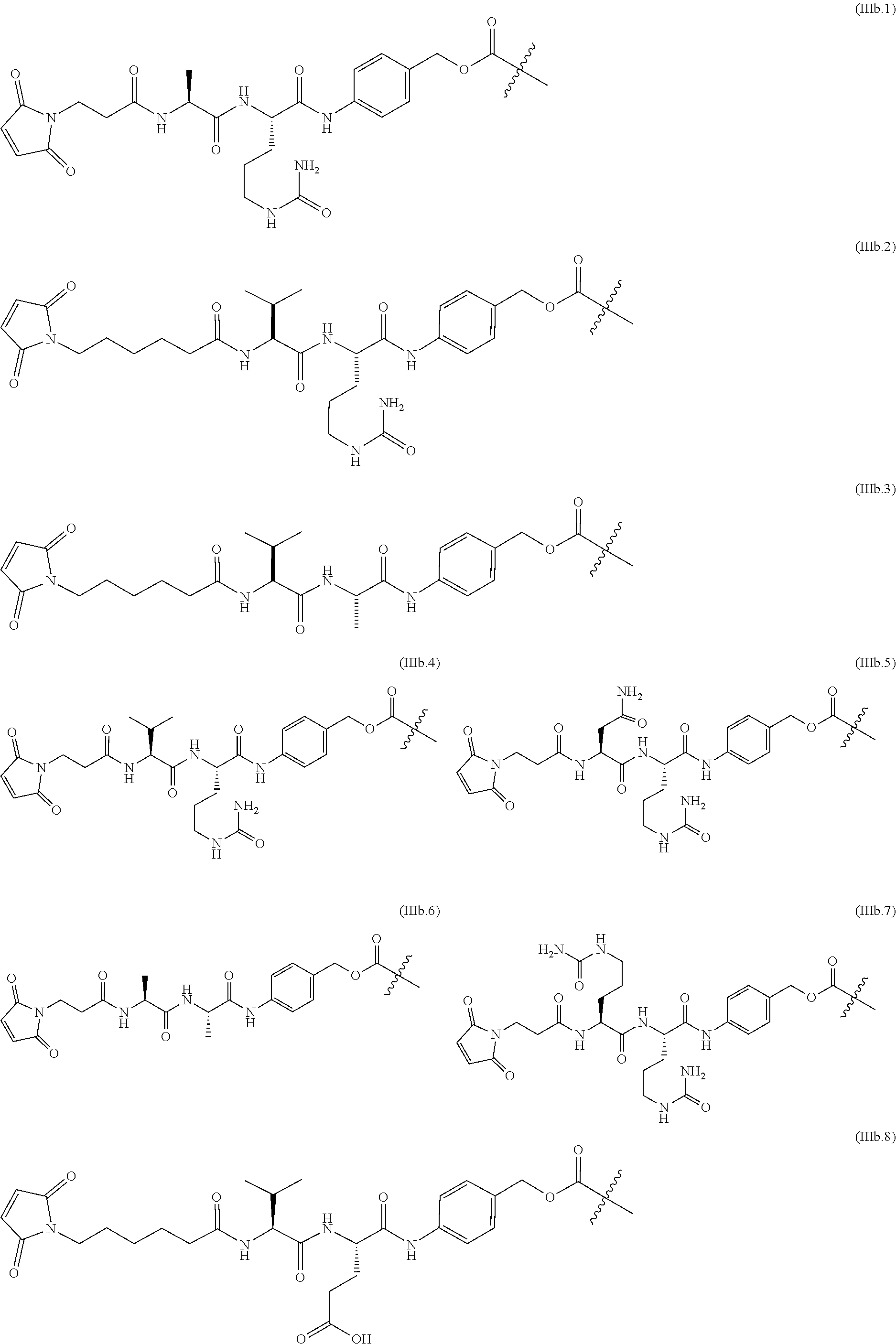

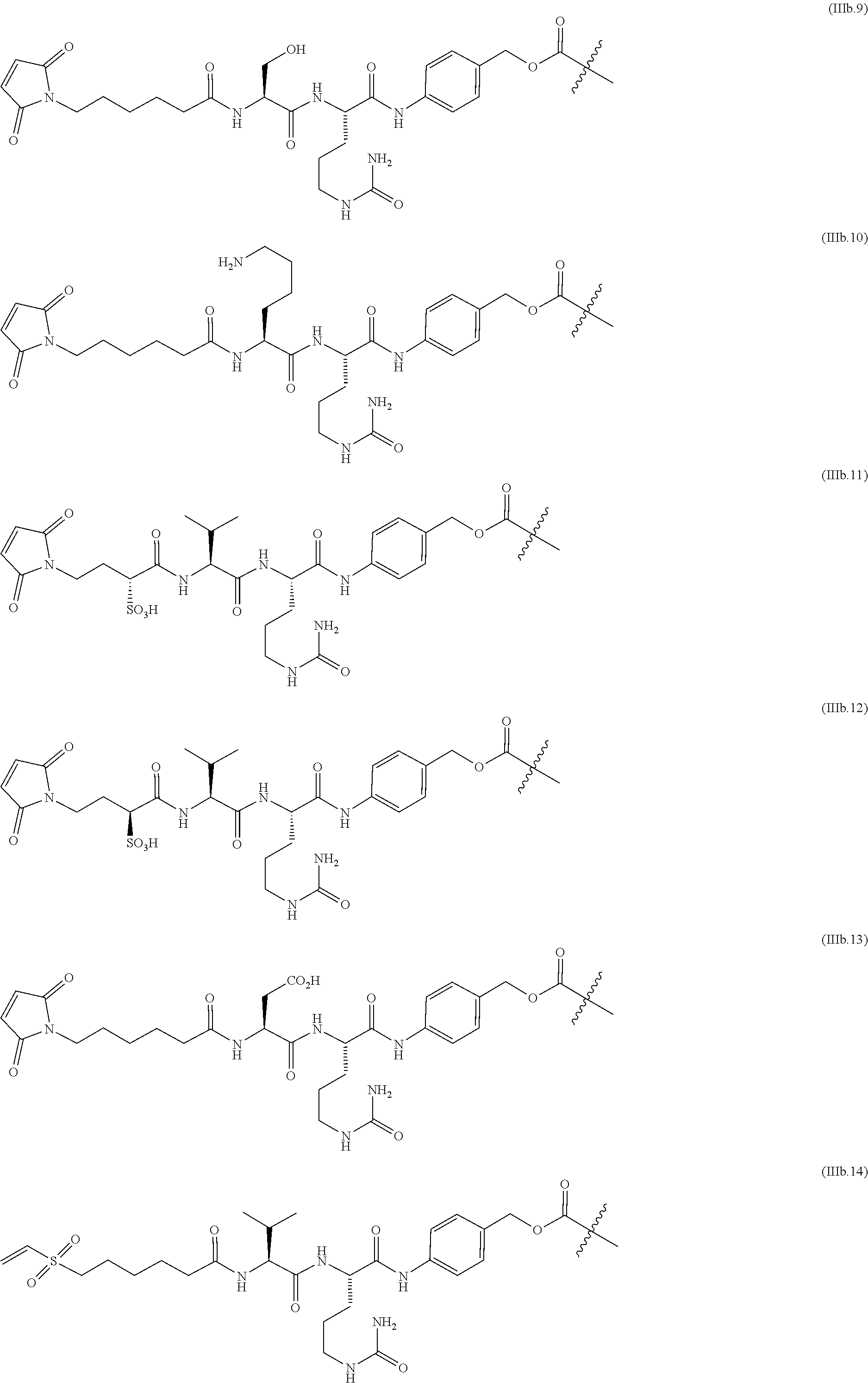

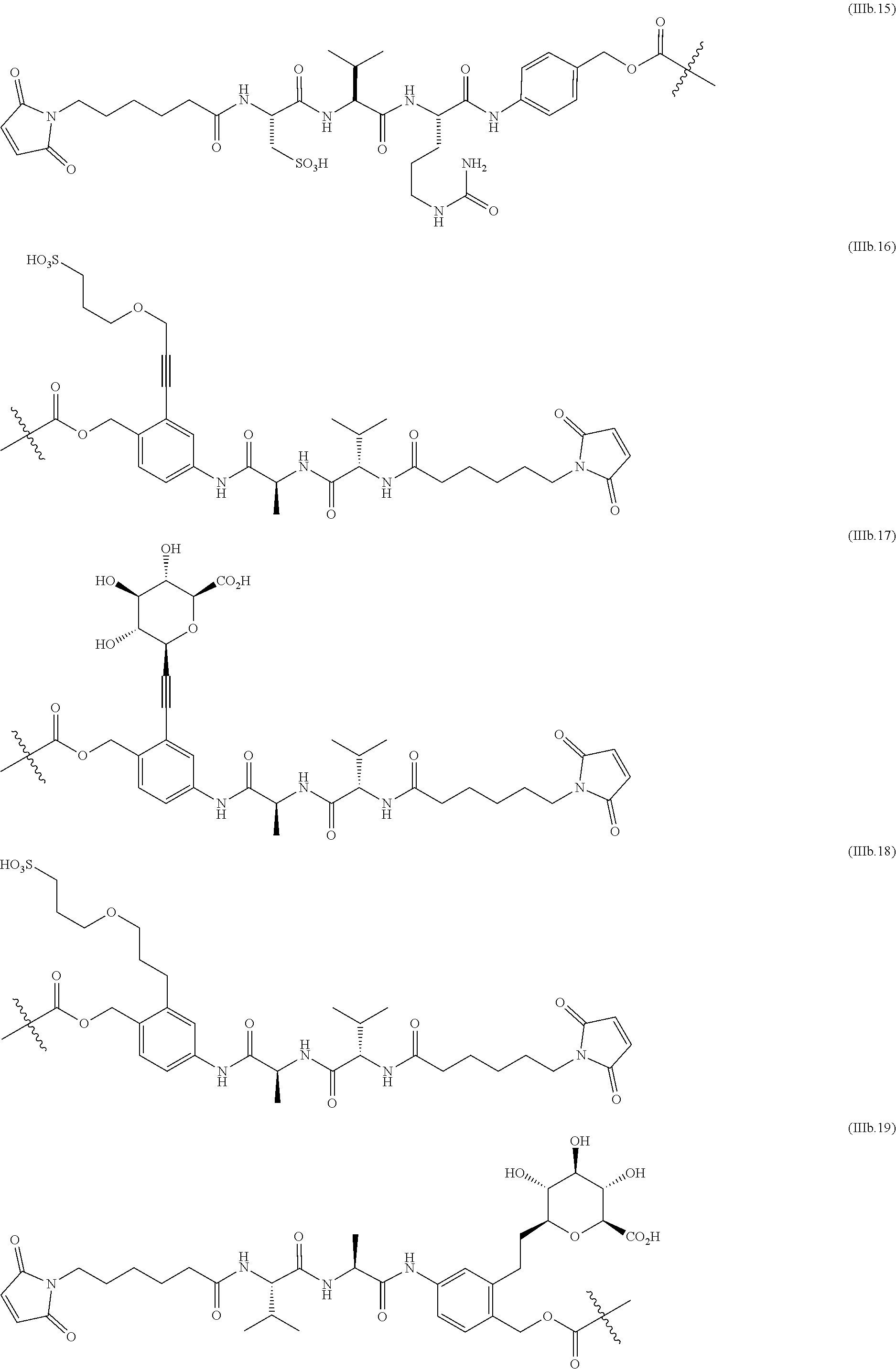

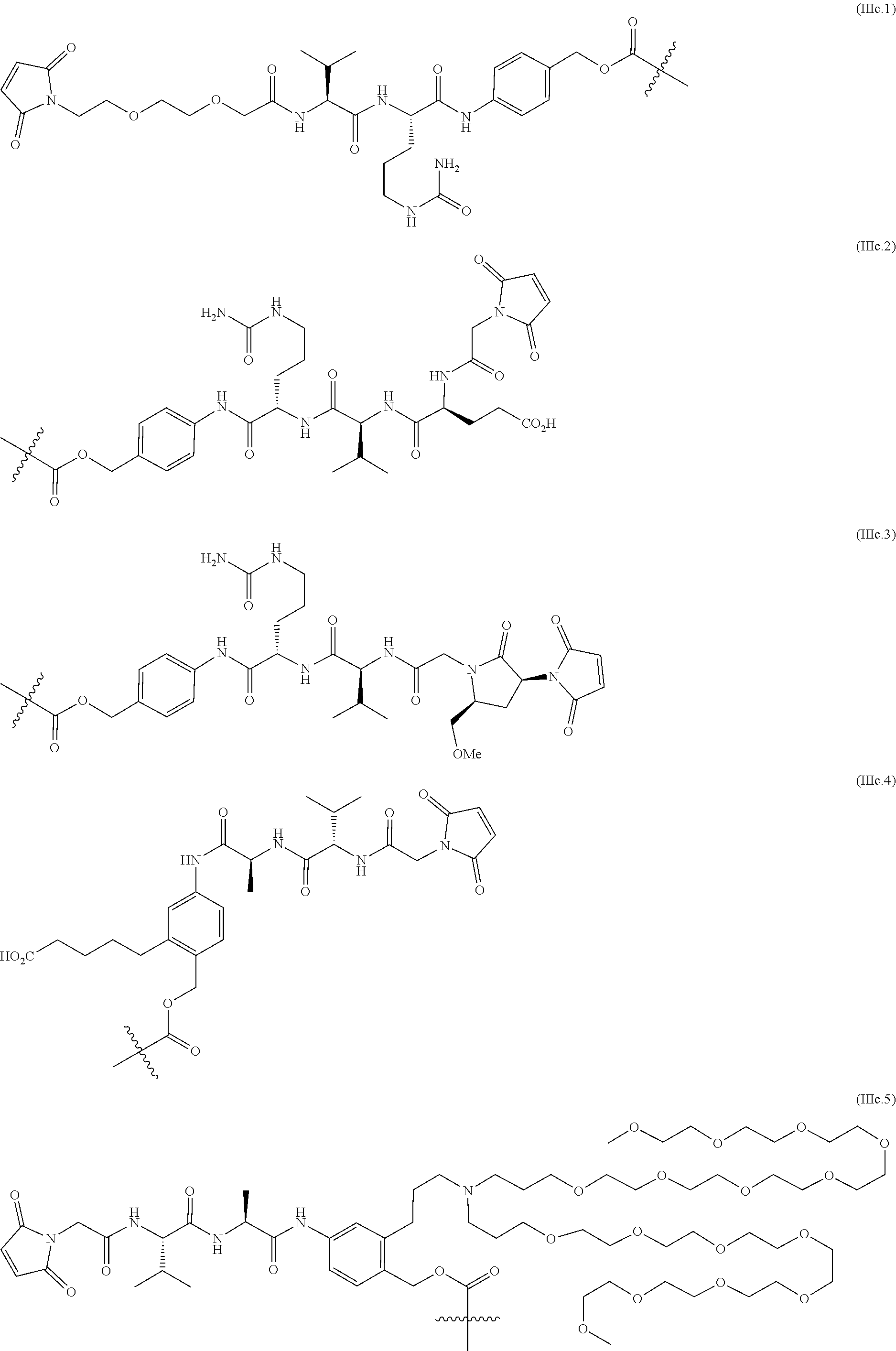

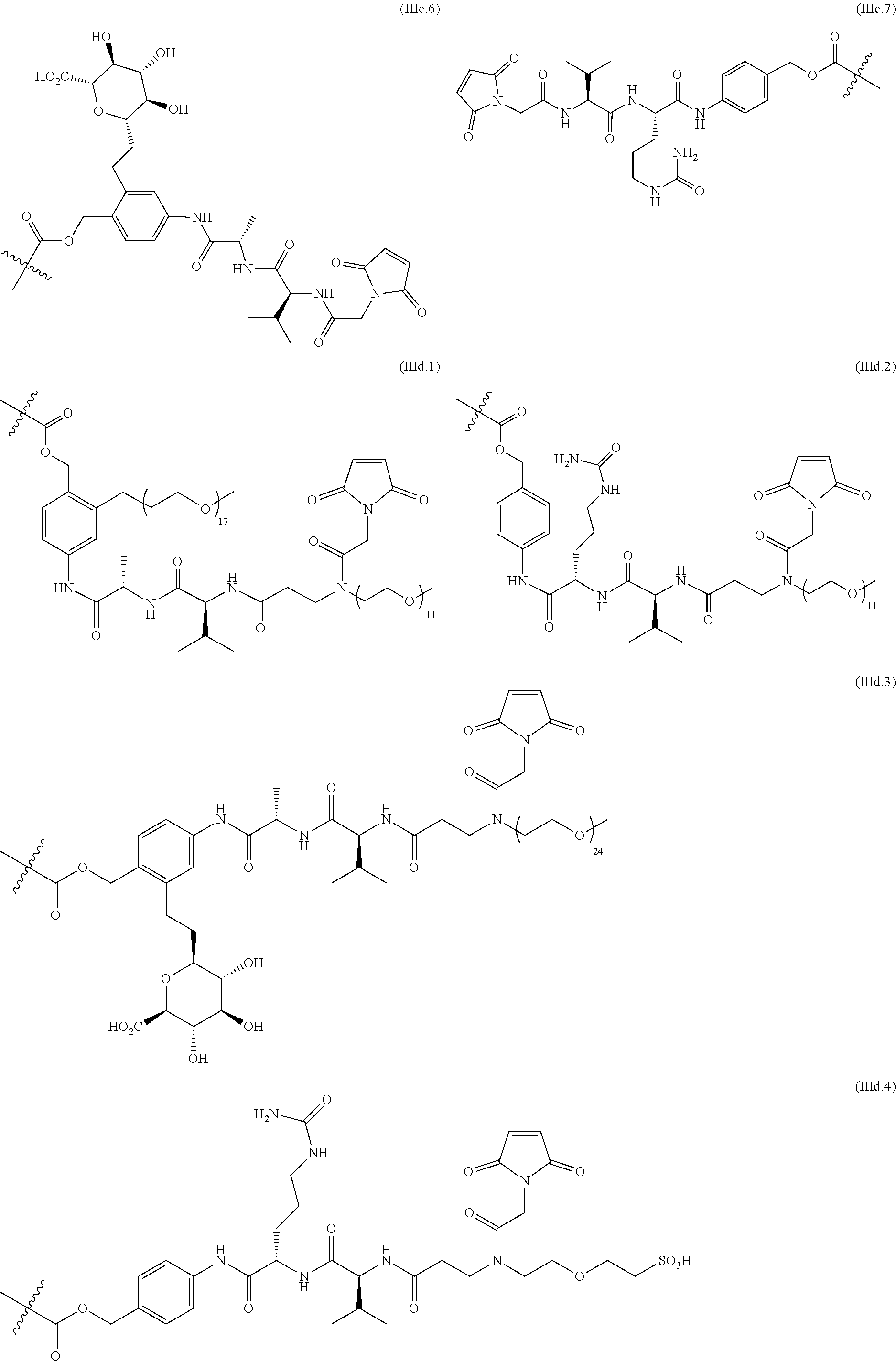

wherein [symbol] indicates an attachment site to an amino-pyrazinecarboxamide compound.

The linker can contain an enzymatically cleavable sugar moiety, for example, a linker comprising structural formula (IVa), (IVb), (IVc), (IVd), or (IVe):

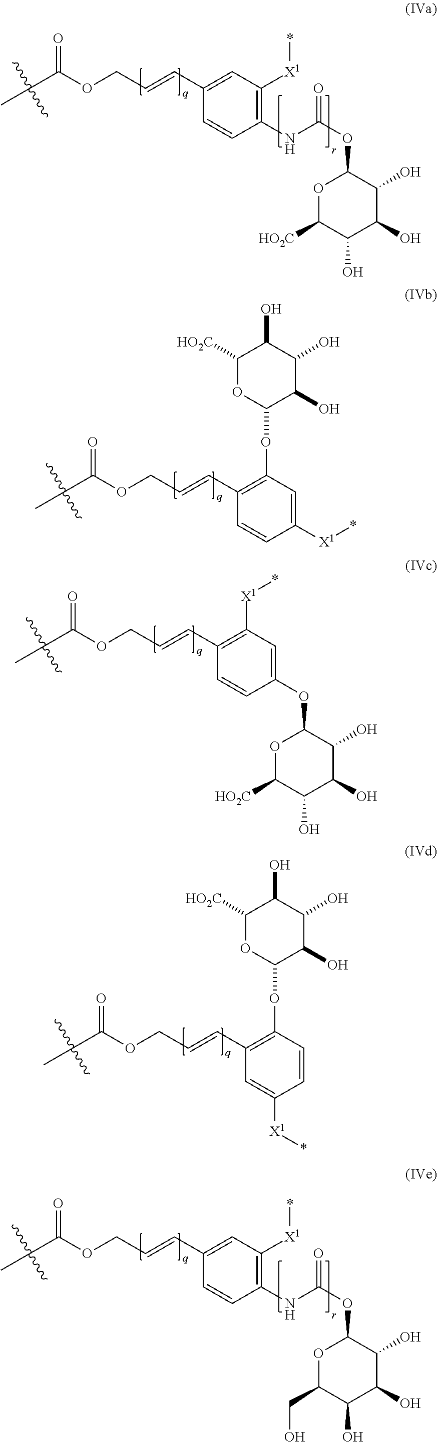

or a salt thereof, wherein: q is 0 or 1; r is 0 or 1; X¹ is CH₂, O or NH; [symbol] represents the point of attachment of the linker (L³) to the compound or salt of any one of Formulas (I-A), (I-B), (I-C), (I-D), (I-E), (II-A), (II-B), (II-C), and (II-D); and * represents the point of attachment to the remainder of the linker.

Exemplary embodiments of linkers according to structural formula (IVa) that may be included in the antibody construct conjugates described herein can include the linkers illustrated below (as illustrated, the linkers include a group suitable for covalently linking the linker to an antibody construct):

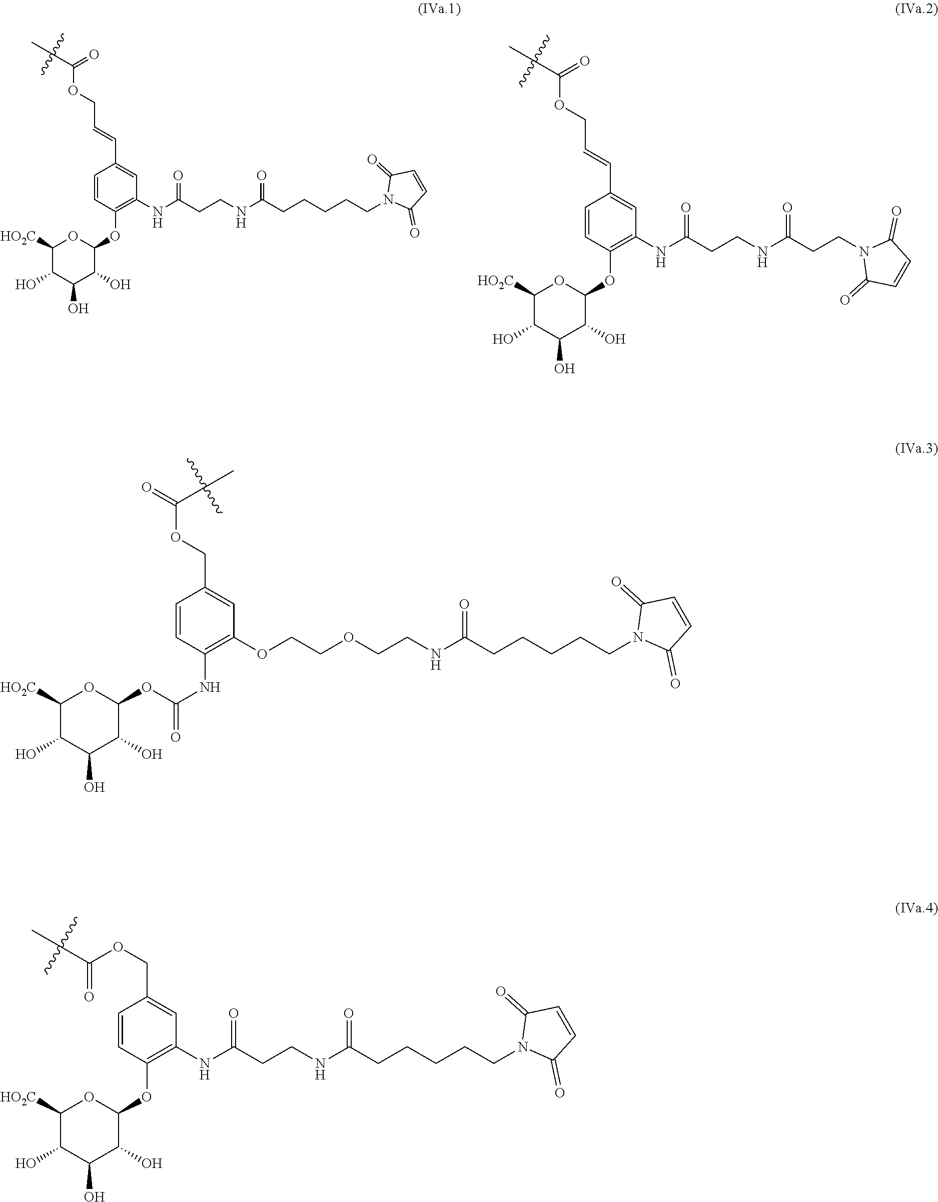

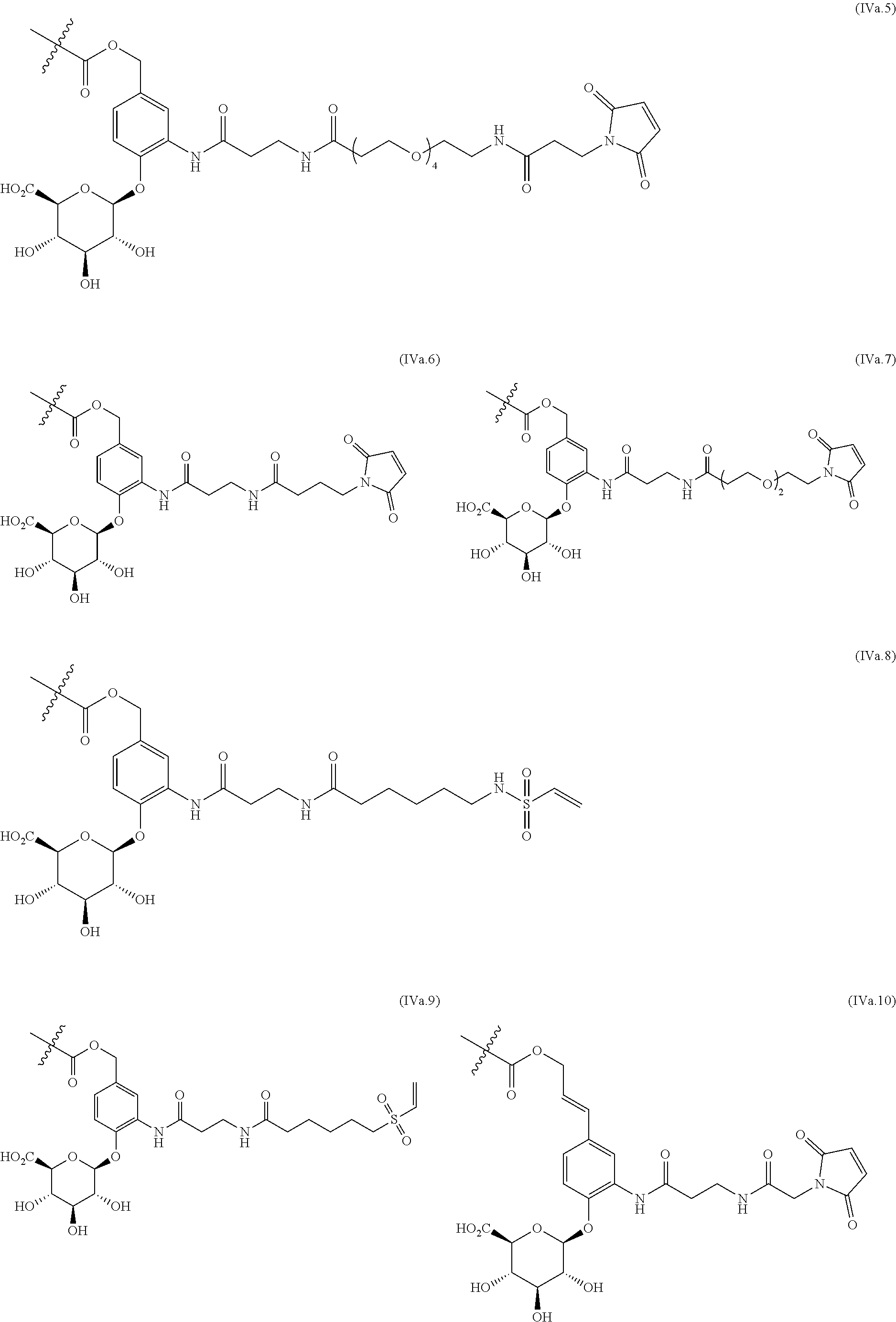

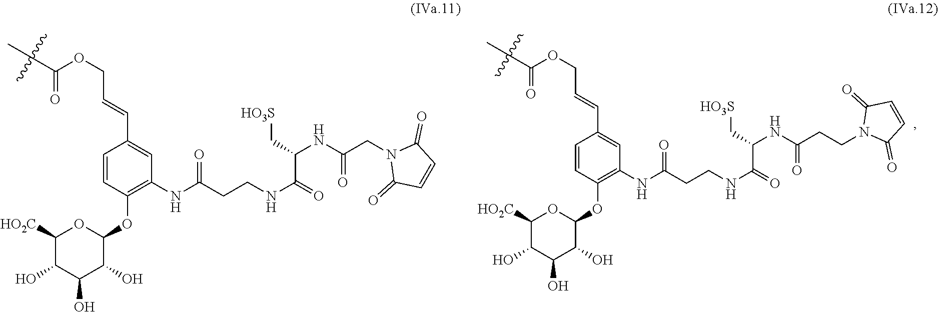

wherein [symbol] represents the point of attachment of the linker (L³) to the compound or salt of any one of Formulas (I-A), (I-B), (I-C), (I-D), (I-E), (II-A), (II-B), (II-C), and (II-D).

Exemplary embodiments of linkers according to structural formula (IVb) that may be included in the conjugates include the linkers illustrated below (as illustrated, the linkers include a group suitable for covalently linking the linker to an antibody construct):

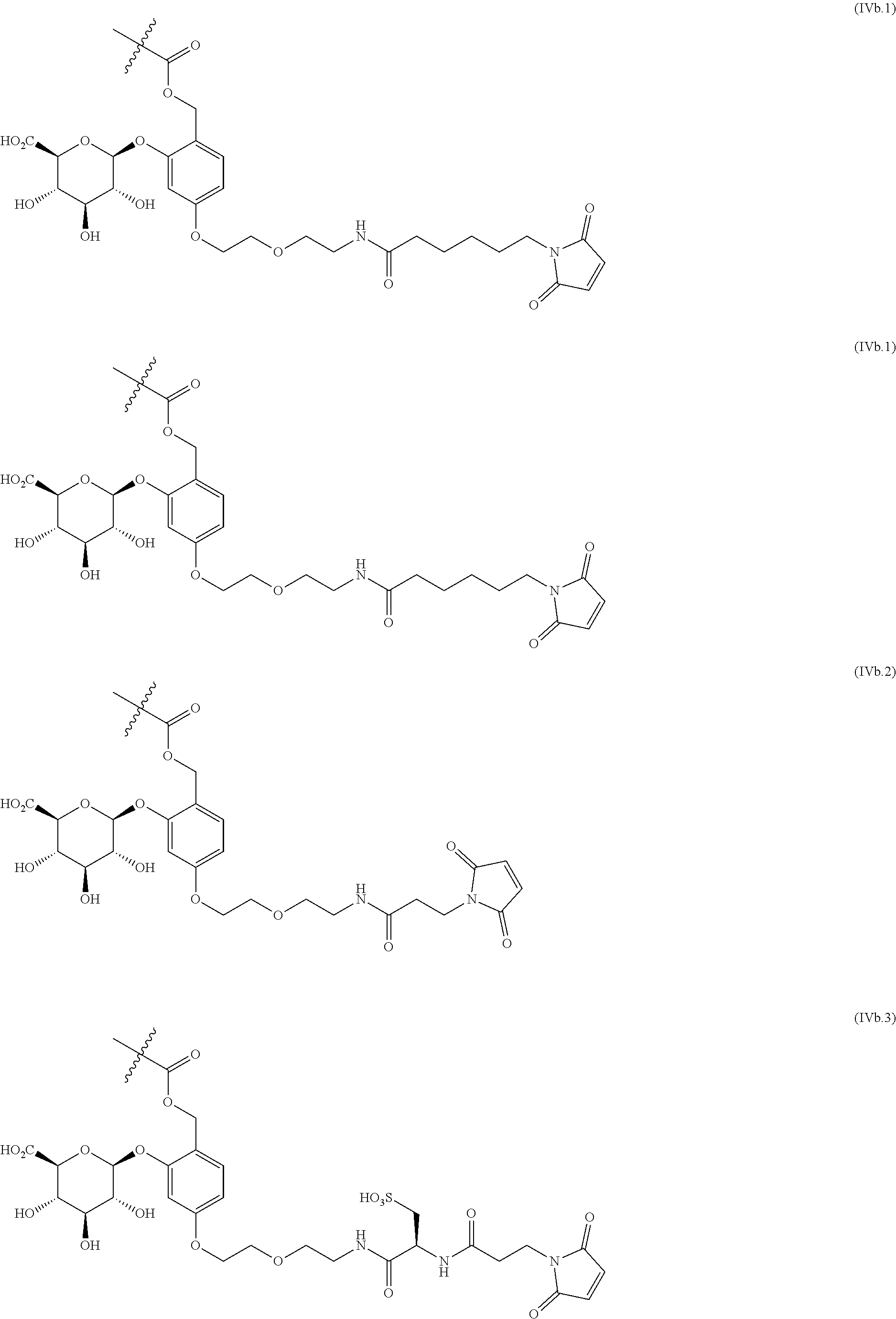

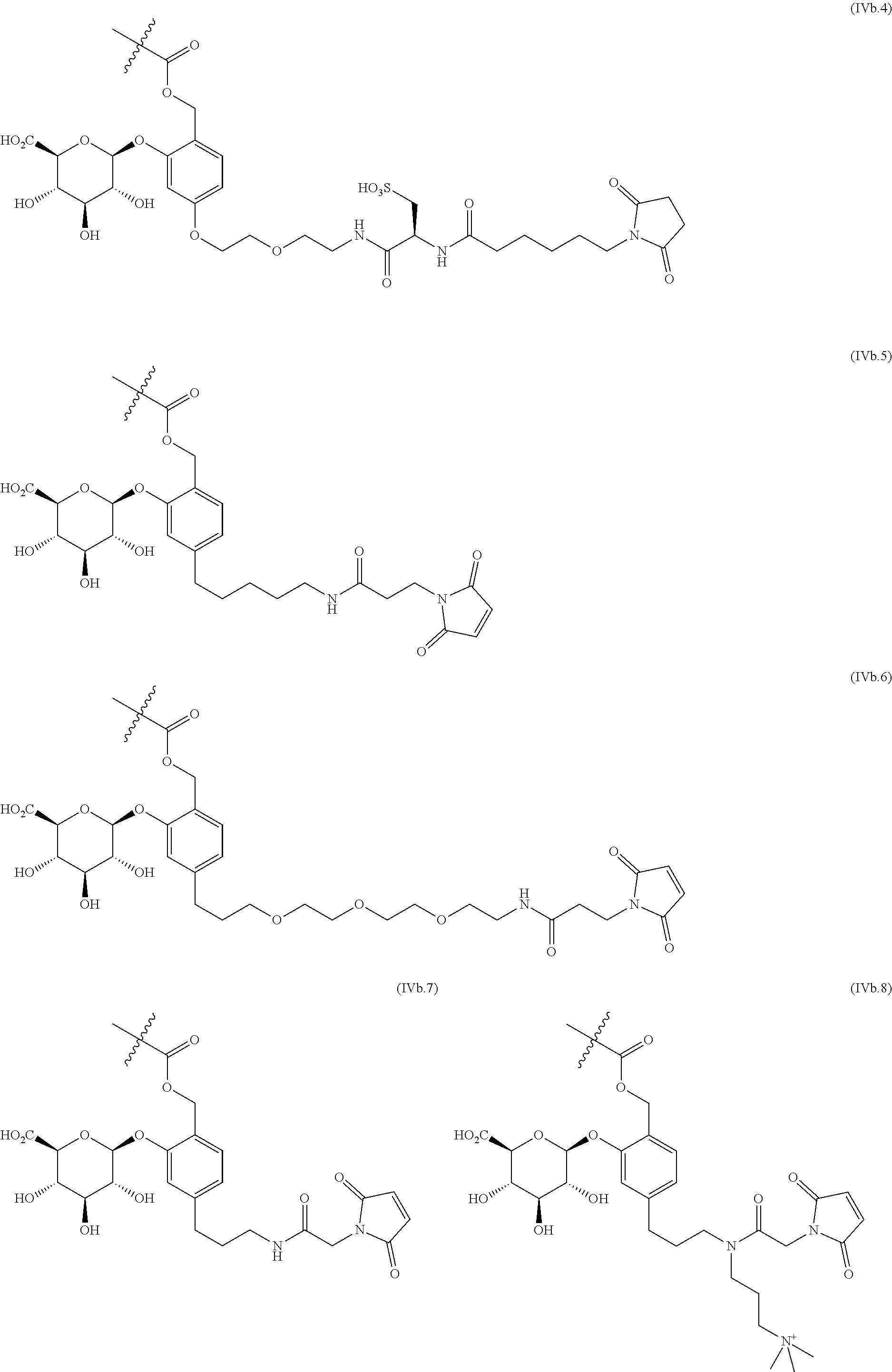

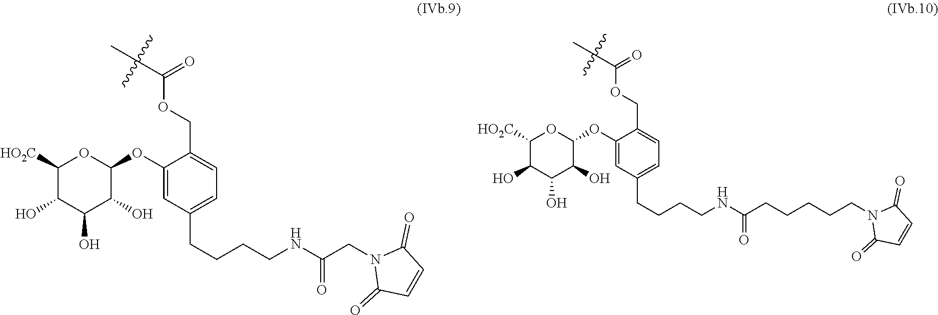

wherein [symbol] represents the point of attachment of the linker (L) to an amino-pyrazinecarboxamide compound.

Exemplary embodiments of linkers according to structural formula (IVc) that may be included in the conjugates include the linkers illustrated below (as illustrated, the linkers include a group suitable for covalently linking the linker to an antibody construct):

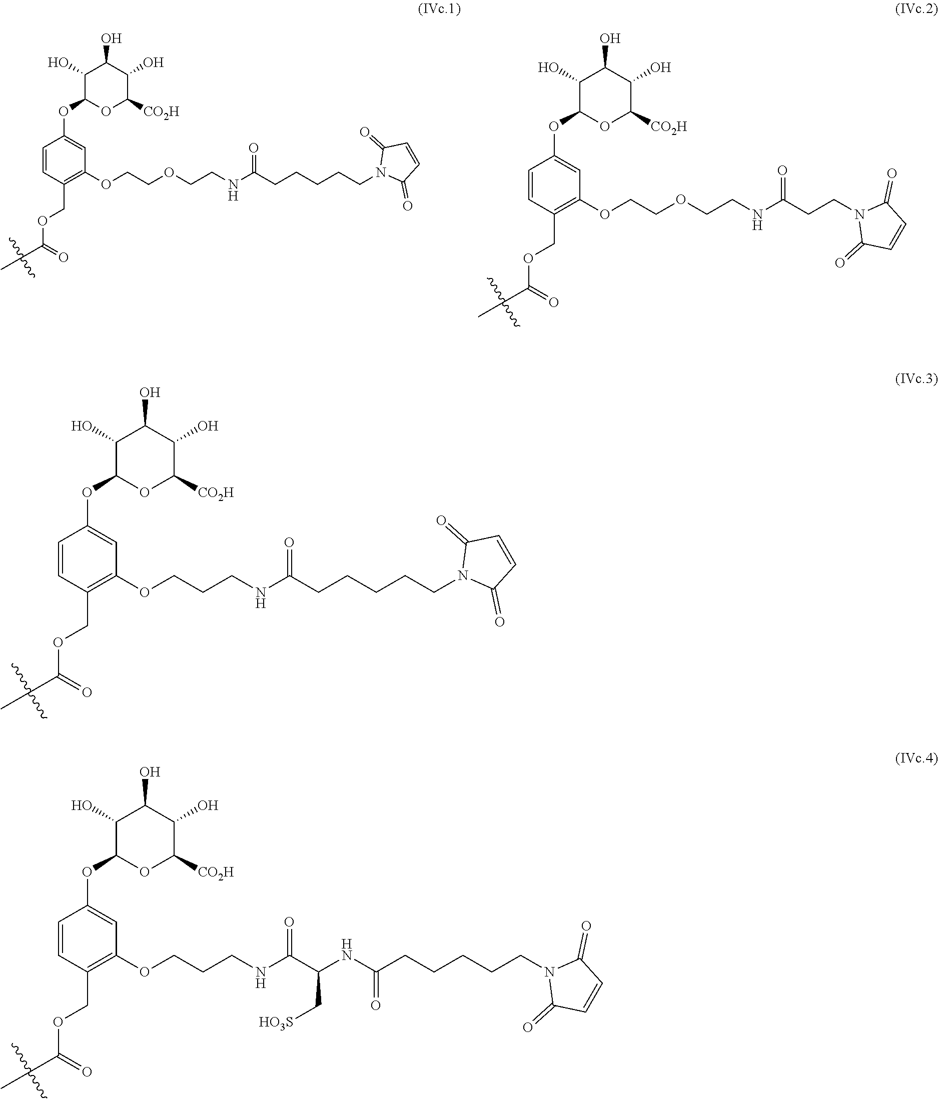

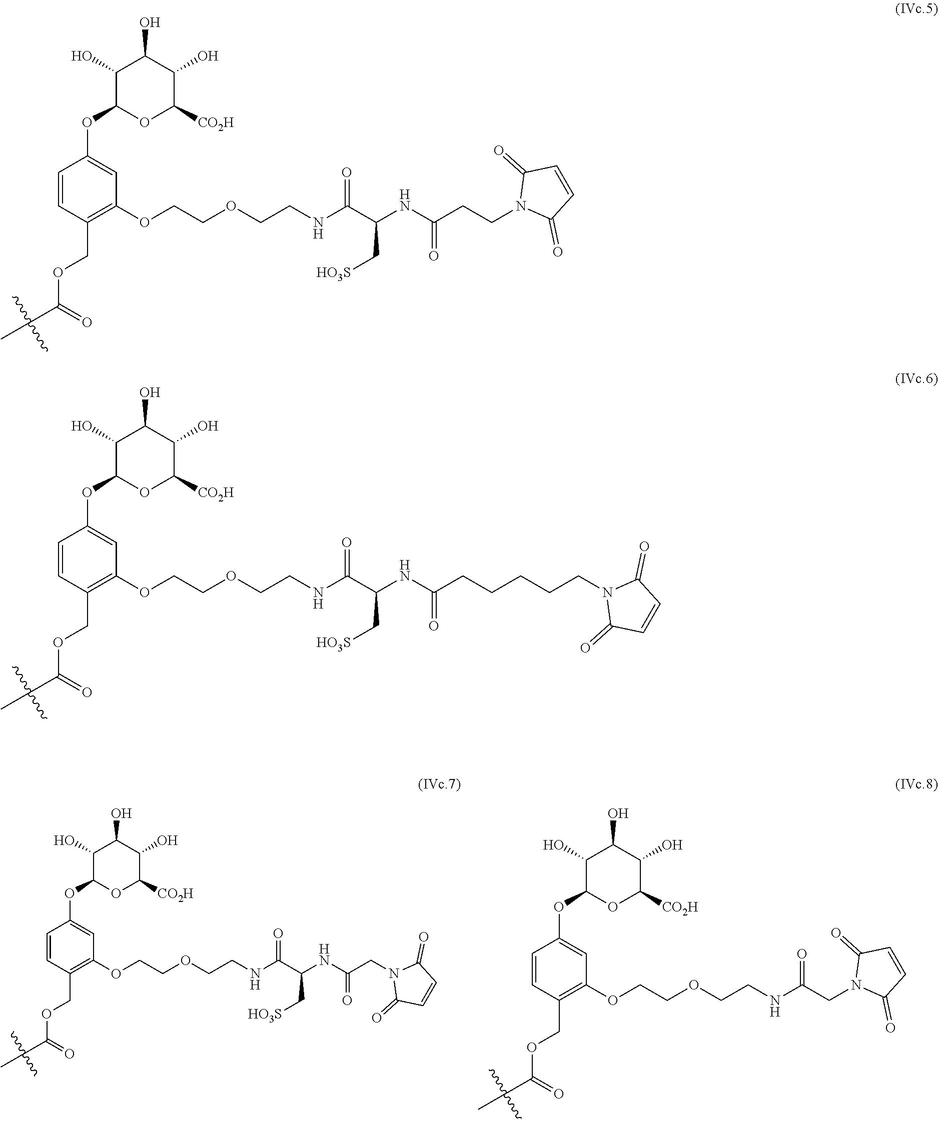

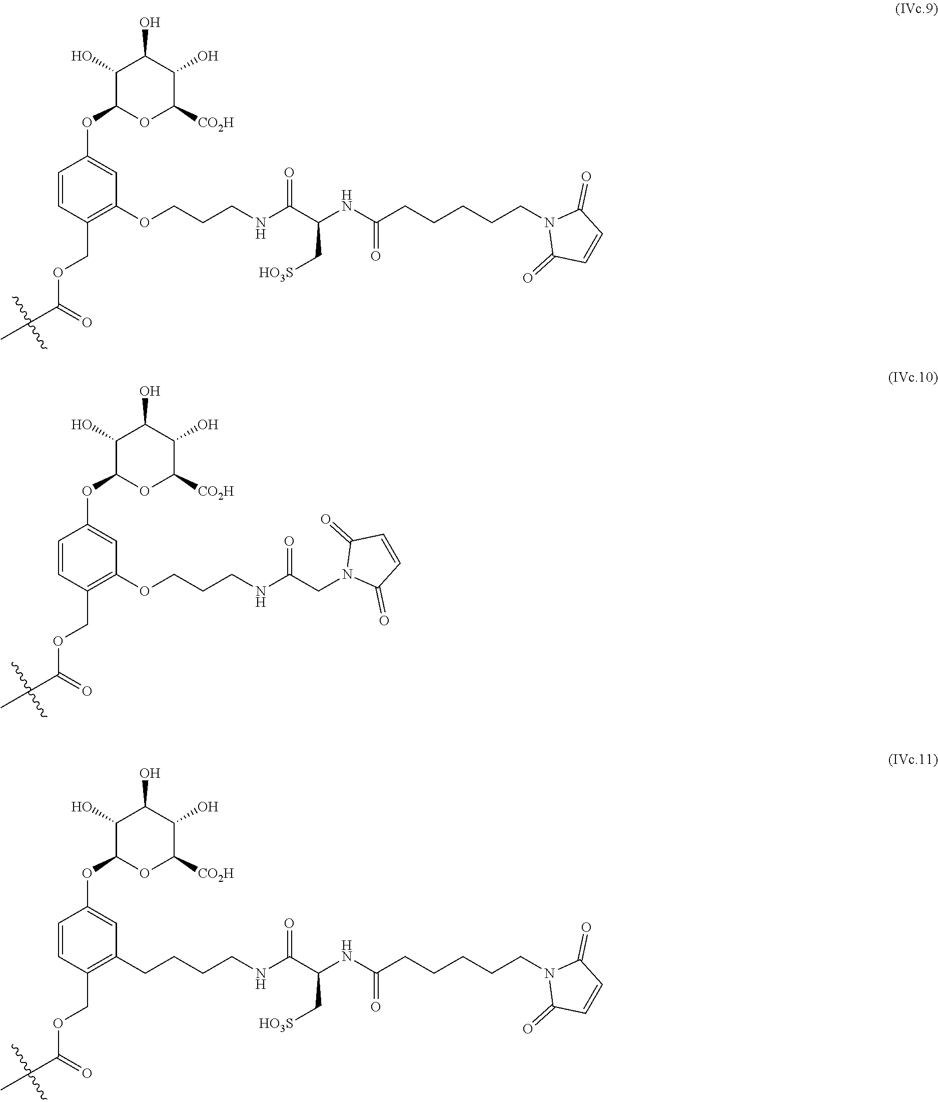

wherein [symbol] represent the point of attachment of the linker (L³) to an amino-pyrazinecarboxamide compound.

Exemplary embodiments of linkers according to structural formula (IVd) that may be included in the conjugates include the linkers illustrated below (as illustrated, the linkers include a group suitable for covalently linking the linker to an antibody):

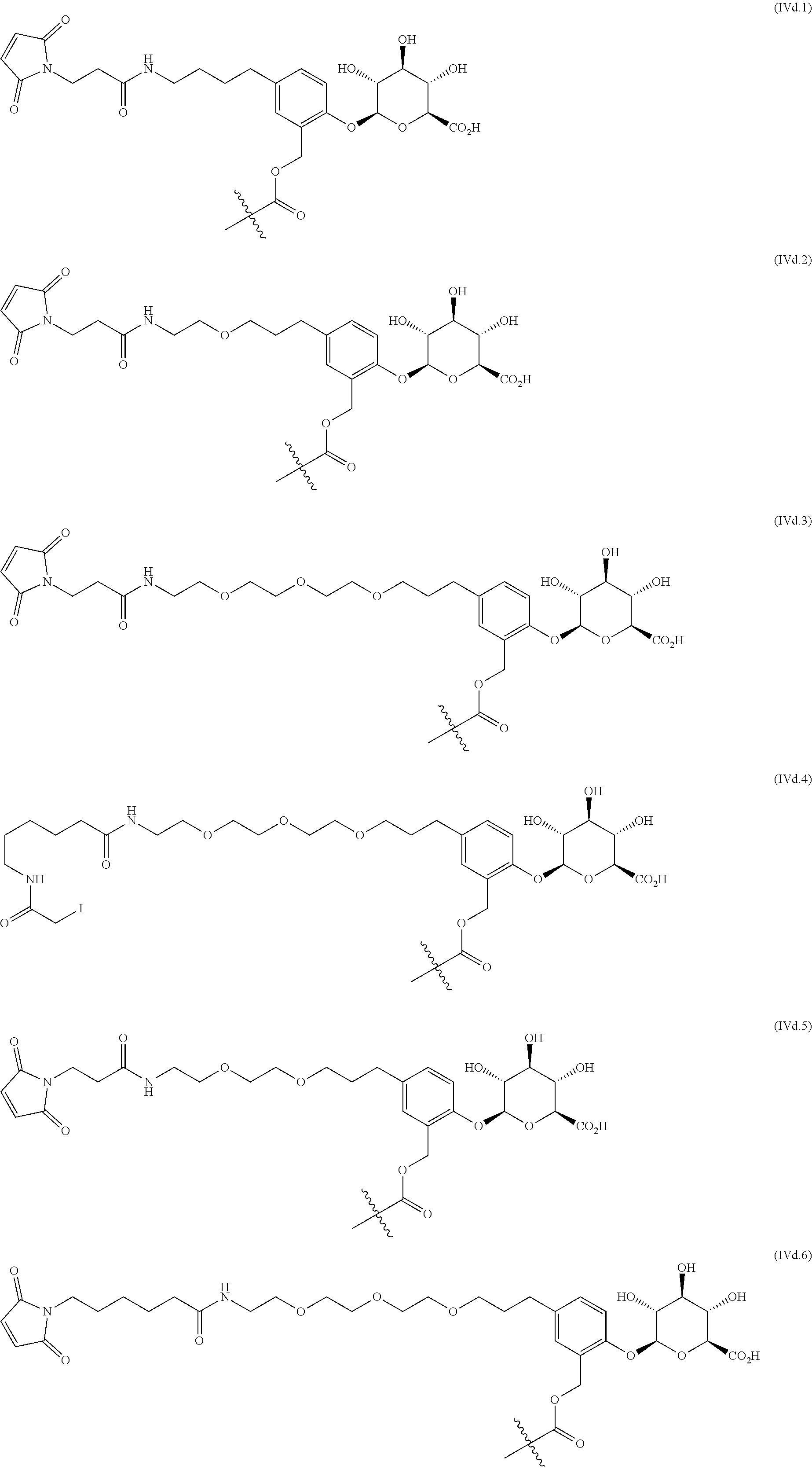

wherein [symbol] represents the point of attachment site of the linker (LC) to an amino-pyrazinecarboxamide compound.

Exemplary embodiments of linkers according to structural formula (IVe) that may be included in the conjugates include the linkers illustrated below (as illustrated, the linkers include a group suitable for covalently linking the linker to an antibody construct):

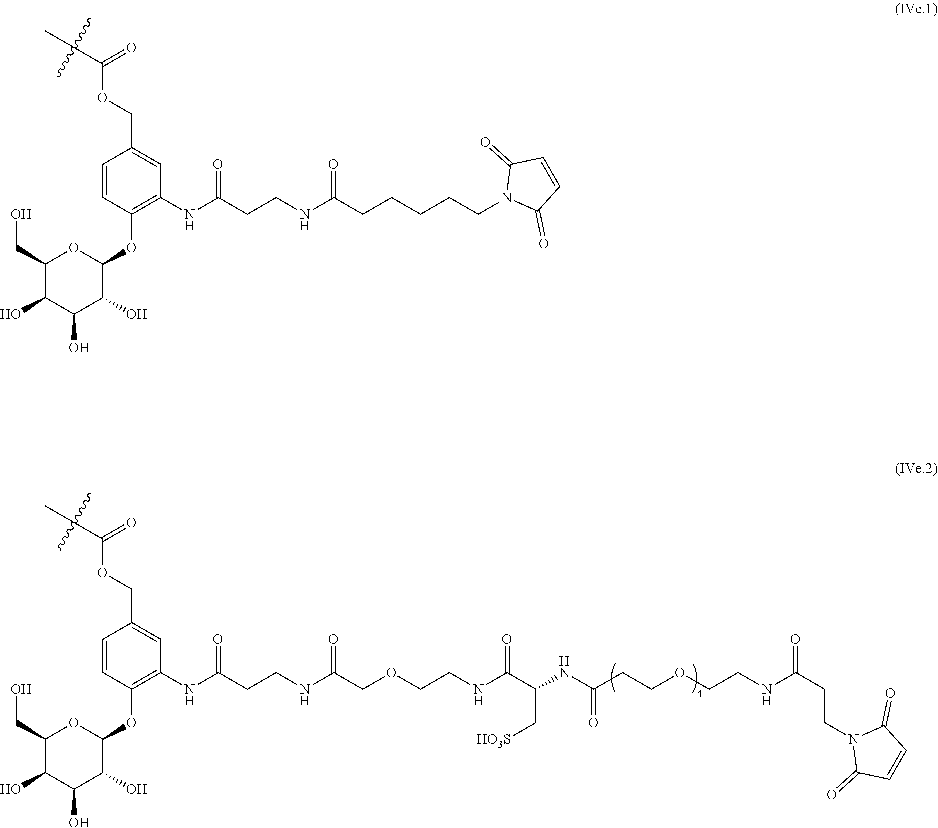

wherein [symbol] represents the point of attachment of the linker (L³) to an amino-pyrazinecarboxamide compound.

Although cleavable linkers can provide certain advantages, the linkers comprising the conjugate need not be cleavable. For non-cleavable linkers, the amino-pyrazinecarboxamide compound release may not depend on the differential properties between the plasma and some cytoplasmic compartments. The release of the amino-pyrazinecarboxamide compound can occur after internalization of the antibody conjugate via antigen-mediated endocytosis and delivery to lysosomal compartment, where the antibody construct can be degraded to the level of amino acids through intracellular proteolytic degradation. This process can release an amino-pyrazinecarboxamide compound derivative (a metabolite of the conjugate containing a non-cleavable linker-heterocyclic compound), which is formed by the amino-pyrazinecarboxamide compound, the linker, and the amino acid residue or residues to which the linker was covalently attached. The payload compound derivative from antibody construct amino-pyrazinecarboxamide compound conjugates with non-cleavable linkers can be more hydrophilic and less membrane permeable, which can lead to less bystander effects and less nonspecific toxicities compared to antibody conjugates with a cleavable linker. Antibody conjugates with non-cleavable linkers can have greater stability in circulation than antibody conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

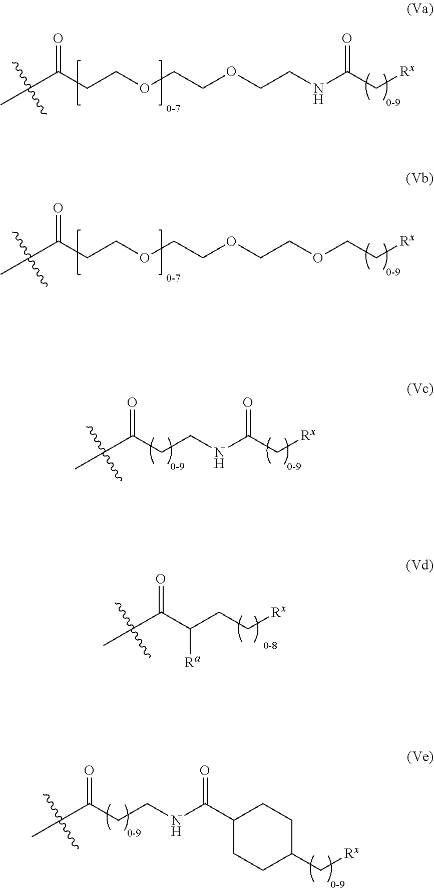

or salts thereof, wherein: R^{a} is selected from hydrogen, alkyl, sulfonate and methyl sulfonate; R^{x} is a reactive moiety including a functional group capable of covalently linking the linker to an antibody construct; and [symbol] represents the point of attachment of the linker (L³) to the compound or salt of any one of Formulas (I-A), (I-B), (I-C), (I-D), (I-E), (II-A), (II-B), (II-C), and (II-D).

Exemplary embodiments of linkers according to structural formula (Va)-(Ve) that may be included in the conjugates include the linkers illustrated below (as illustrated, the linkers include a group suitable for covalently linking the linker to an antibody construct, and [symbol] represents the point of attachment of the linker (L³) to the compound or salt of any one of Formulas (I-A), (I-B), (I-C), (I-D), (I-E), (II-A), (II-B), (II-C), and (II-D):

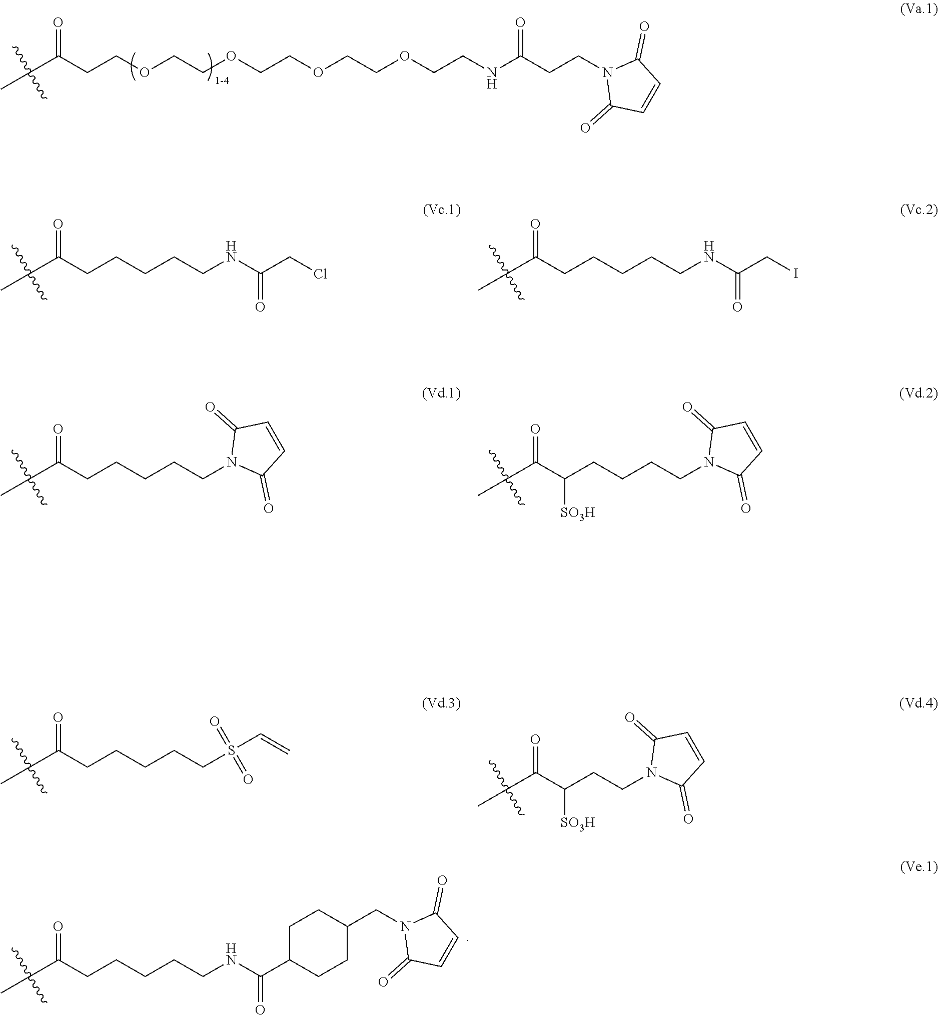

Attachment groups that are used to attach the linkers to an antibody construct can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody of a conjugate. The reaction between a thiol group of an antibody and a drug with a linker including a maleimide group proceeds according to the following scheme:

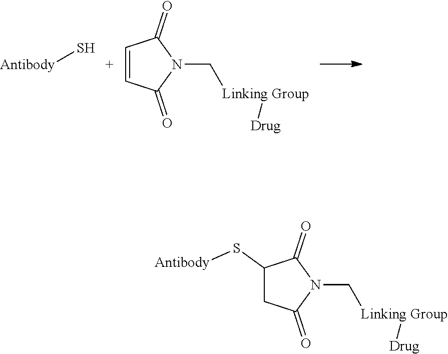

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic

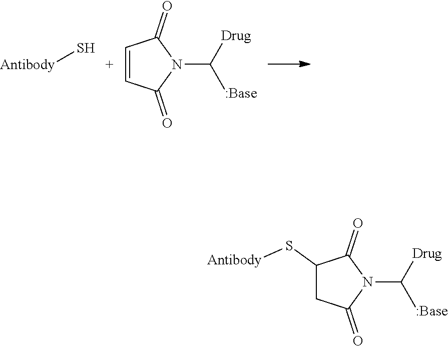

is shown below:

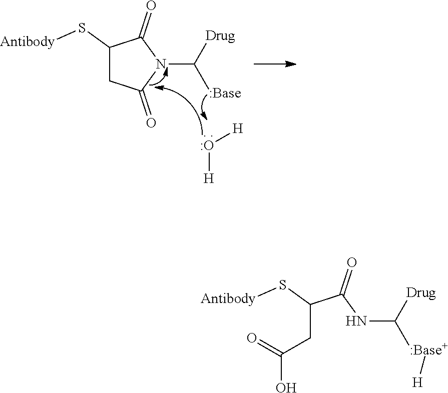

The hydrolysis reaction schematically represented above may occur at either carbonyl group of the succinimide group. Accordingly, two possible isomers may result, as shown below:

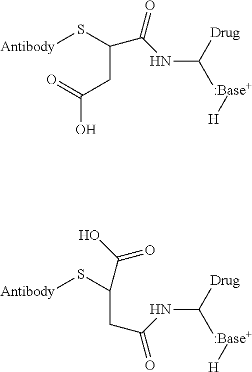

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker, e.g., any L³ with a maleimide group prior to conjugation to an antibody construct may facilitate hydrolysis of a nearby succinimide group formed after conjugation of the antibody construct to the linker. Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH₂)ₘ—, where m is an integer from 0 to 10. An alkylene chain may be optionally substituted with other functional groups as described herein.

A linker (L³) with a maleimide group may include an electron withdrawing groups such as, but not limited to, —C(O)R, ═O, —CN, —NO₂, —CX₃, —X, —COOR, —CONR₂, —COR, —COX, —SO₂R, —SO₂OR, —SO₂NHR, —SO₂NR₂, —PO₃R², —P(O)(CH₃)NHR, —NO, —NR₃+, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups such as those described herein.

Examples of self-stabilizing linkers are provided in, e.g., U.S. Patent Publication Number 2013/0309256, the linkers of which are incorporated by reference herein. It will be understood that a self-stabilizing linker useful in conjunction with the compounds of the present invention may be equivalently described as unsubstituted maleimide-including linkers, thio-substituted succinimide-including linkers, or hydrolyzed, ring-opened thio-substituted succinimide-including linkers.

In certain embodiments, a linker of the disclosure (L³) comprises a stabilizing group selected from:

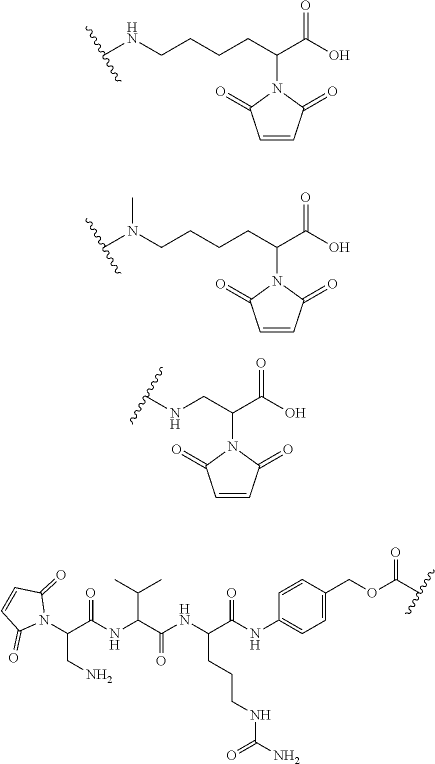

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represent the point of attachment to compound or salt of any one of Formulas (I-A), (I-B), (I-C), (I-D), (I-E), (II-A), (II-B), (II-C), and (II-D).

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls from interchain disulfides) followed by reaction with 4 equivalents of the alkylating agent. Conjugates containing “bridged disulfides” are also claimed to have increased stability.

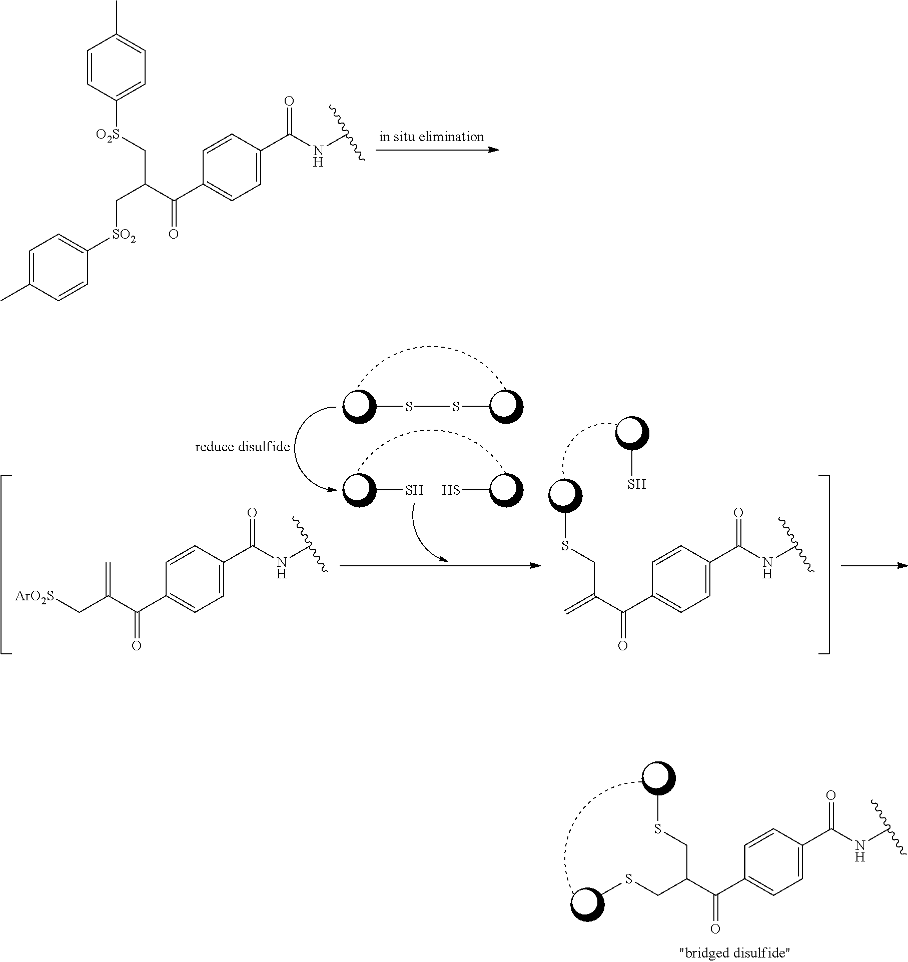

Similarly, as depicted below, a maleimide derivative that is capable of bridging a pair of sulfhydryl groups has been developed.

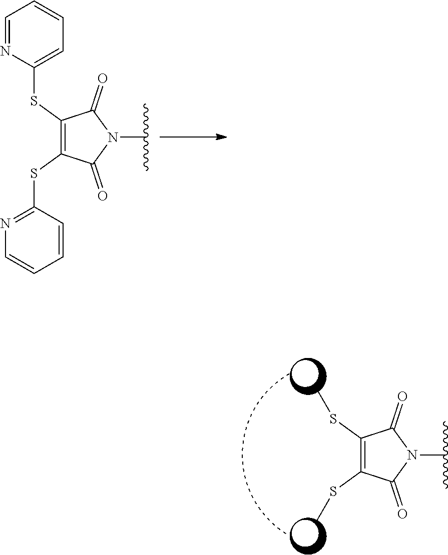 [SMILES: *N1C(=O)C(Sc2ccccn2)=C(Sc2ccccn2)C1=O ; *N1C(=O)C(SC)=C(SC)C1=O]

A linker of the disclosure, L³, can contain the following structural formulas (VIa), (VIb), or (VIc):

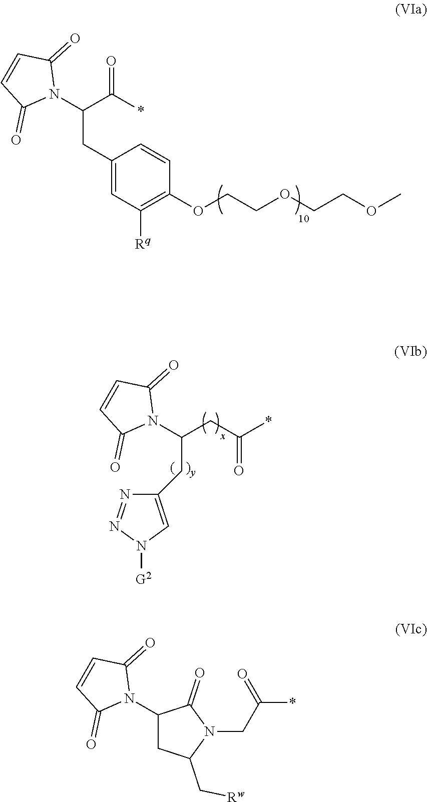

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIa) and (VIb) that can be included in the conjugates can include the linkers illustrated below (as illustrated, the linkers include a group suitable for covalently linking the linker to an antibody construct):

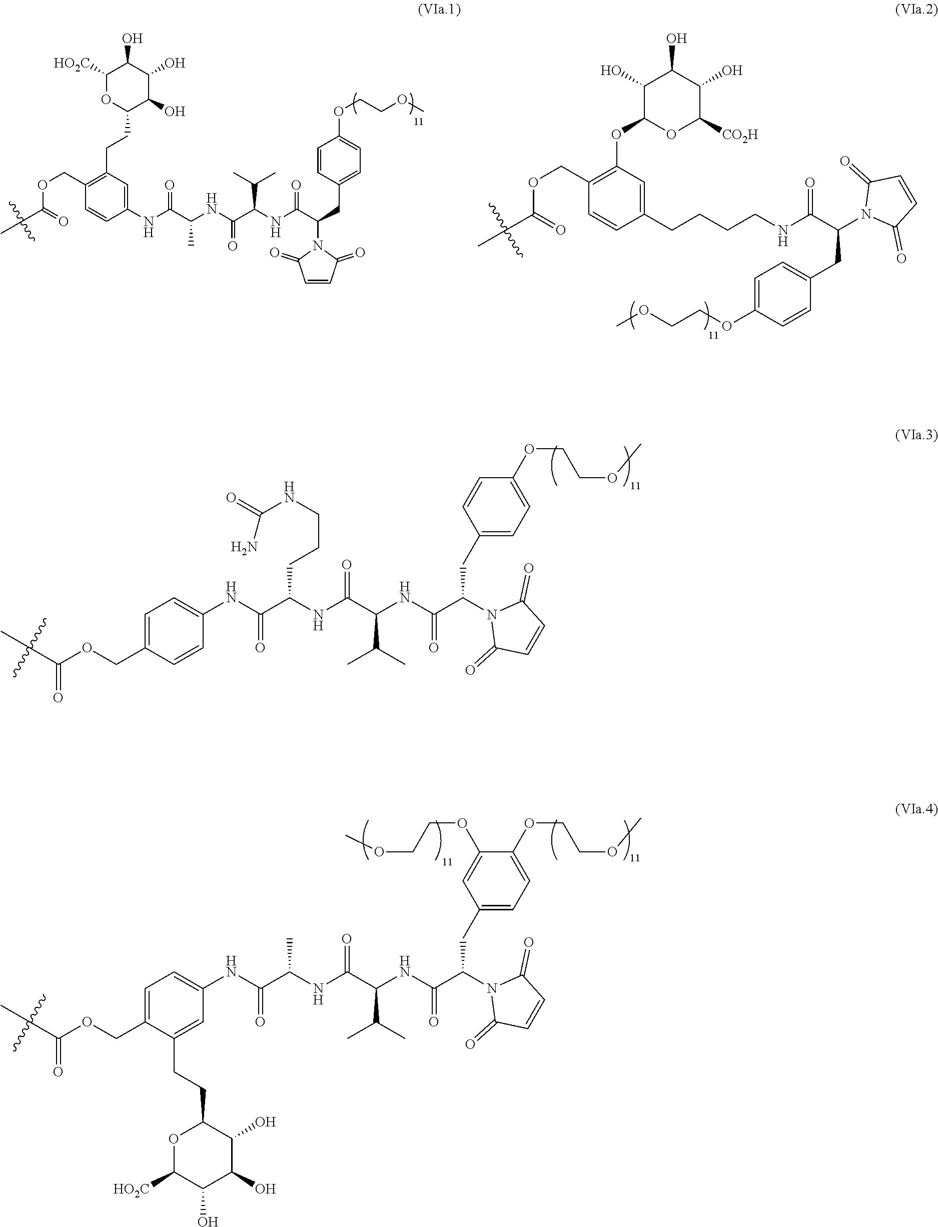

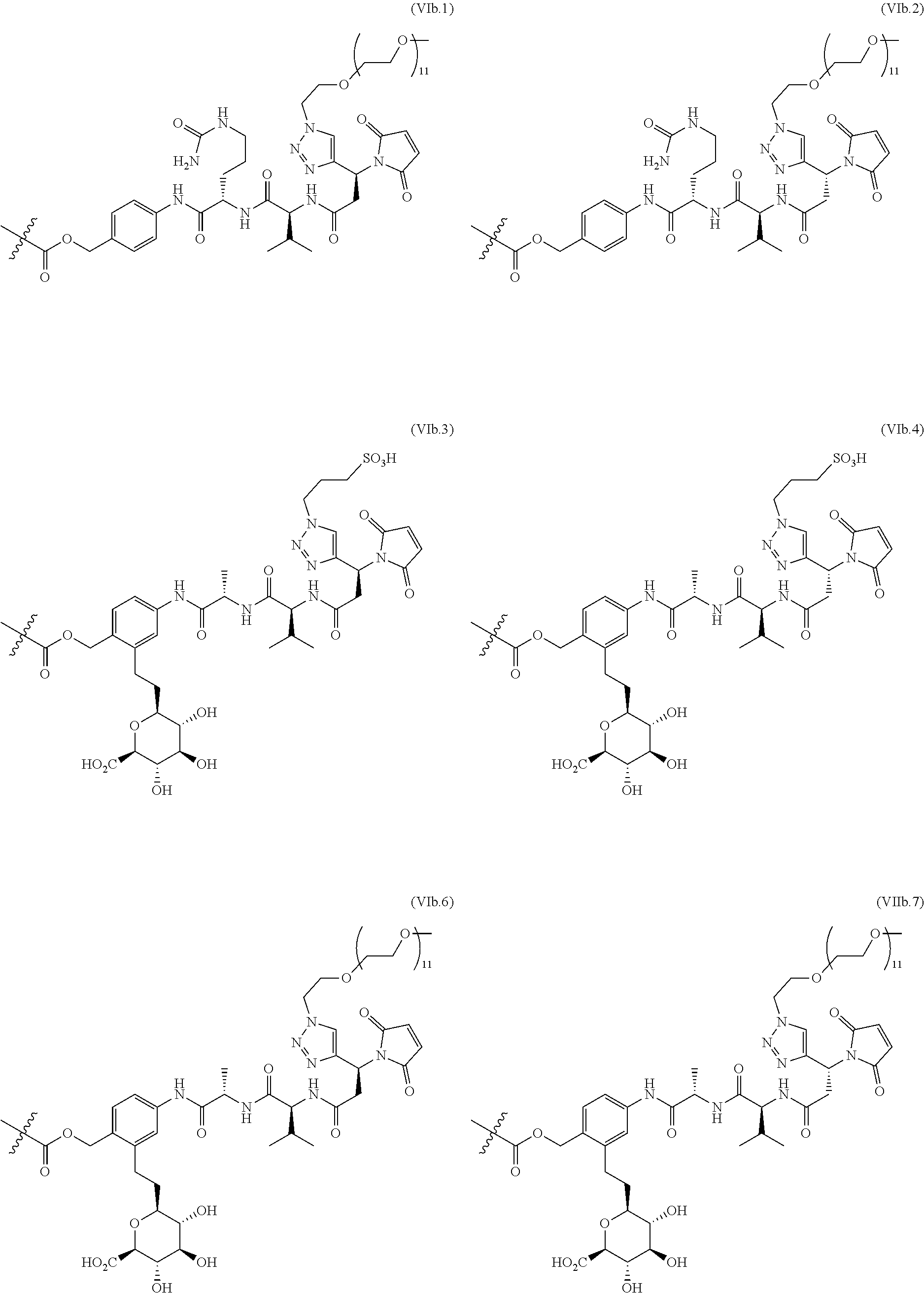

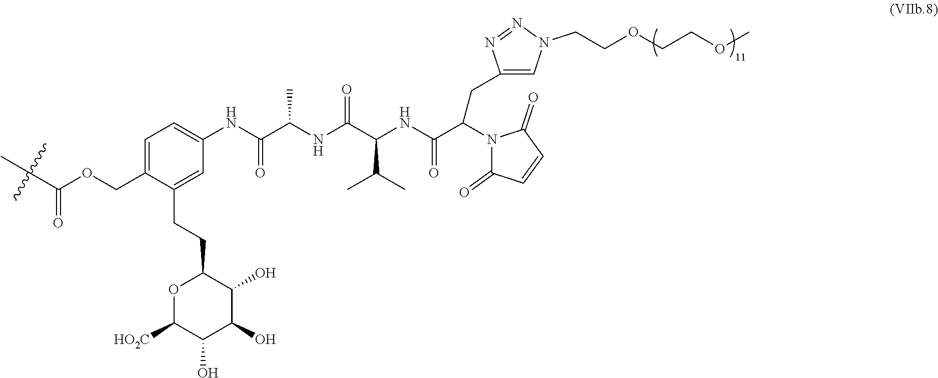

wherein [symbol] represents the point of attachment of the linker (L) to the compound or salt of any one of Formulas (I-A), (I-B), (I-C), (I-D), (I-E), (II-A), (II-B), (II-C), and (II-D).

Exemplary embodiments of linkers according to structural formula (VIc) that can be included in the antibody construct conjugates can include the linkers illustrated below (as illustrated, the linkers include a group suitable for covalently linking the linker to an antibody construct):

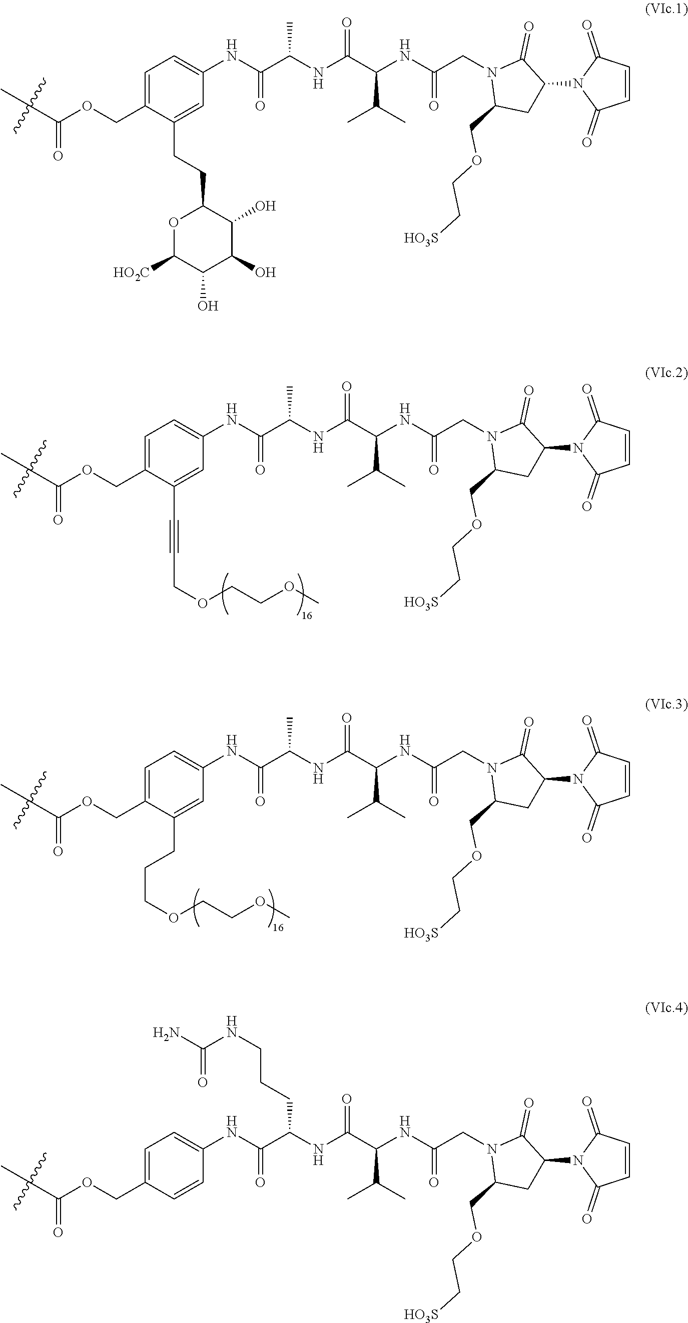

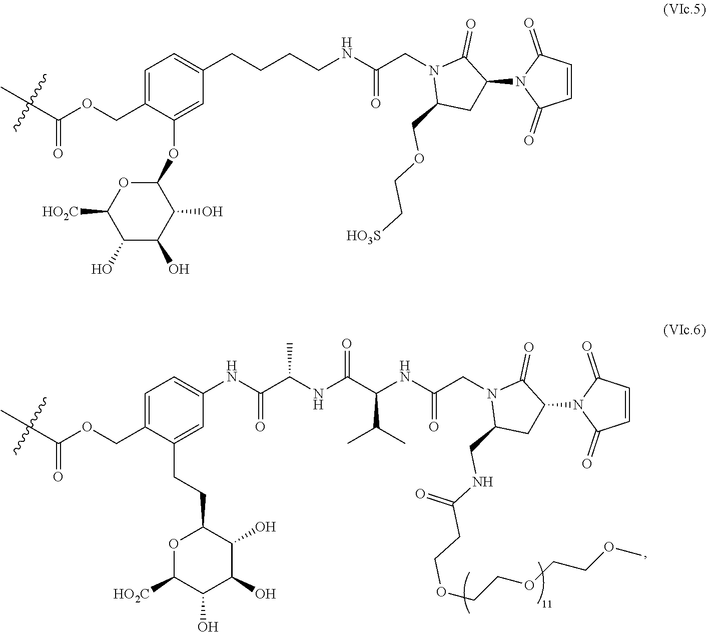

wherein [symbol] represents the point of attachment of the linker (L³) to the compound or salt of any one of Formulas (I-A), (I-B), (I-C), (I-D), (I-E), (II-A), (II-B), (II-C), and (II-D).

Some exemplary linkers (L³) are described in the following paragraphs. In some embodiments for a compound or salt of Formula (I-A), (I-B), (I-C), (I-D), (I-E), (II-A), (II-B), (II-C), and (II-D) and Table 14 wherein attachment of the linker is to a nitrogen of the compound and conjugation is to a cysteine residue of an antibody or targeting moiety, -L³ is represented by the formulas set forth in Table 3 below:

TABLE 3

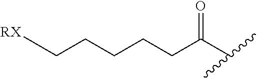

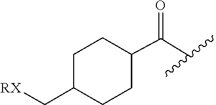

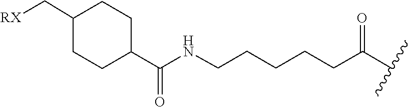

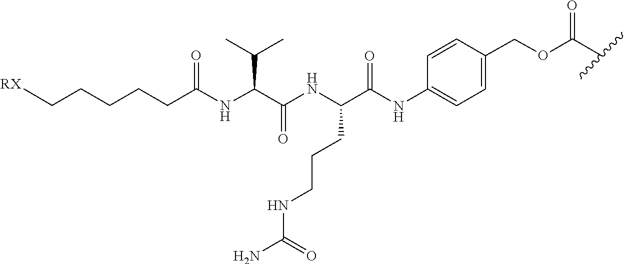

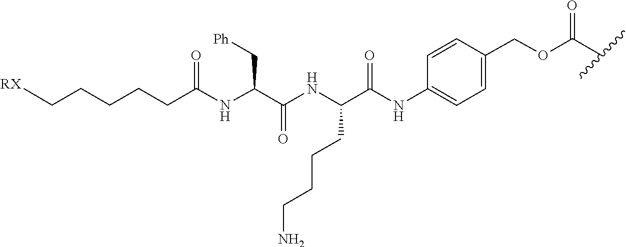

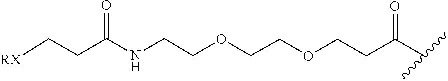

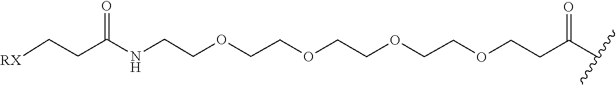

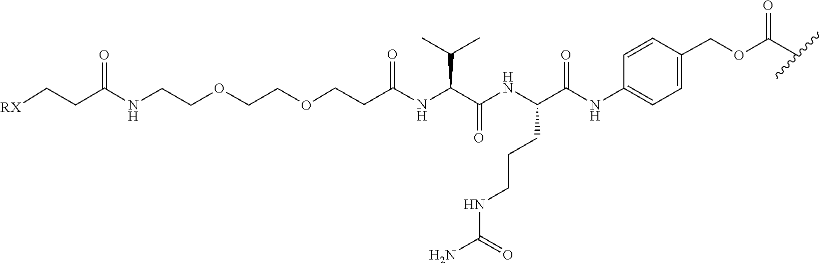

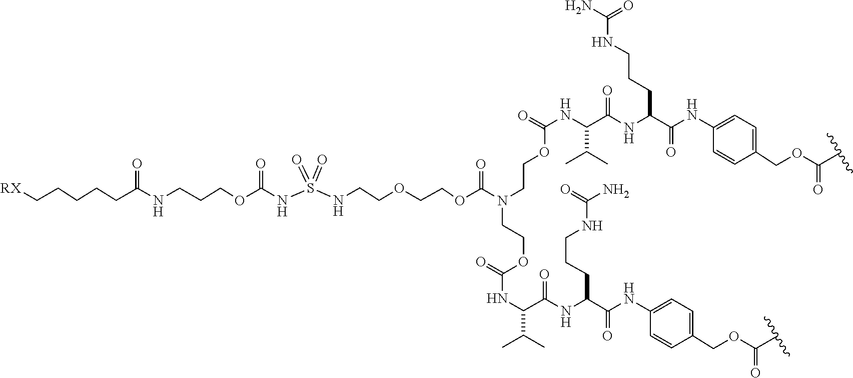

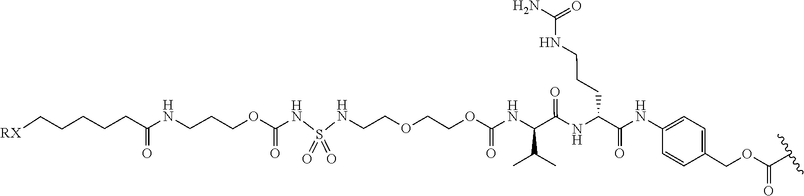

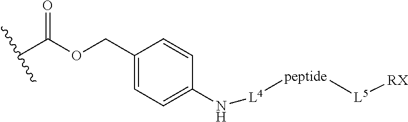

L⁴ represents the C-terminus of the peptide and L⁵ is selected from a bond, alkylene and heteroalkylene, wherein L⁵ is optionally substituted with one or more groups independently selected from R³⁰, and R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurence with one or more substituents selected from halogen, —OH, —CN, —O-alkyl, —SH, ═O, ═S, —NH₂, and -—NO₂.

wherein

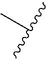

represents attachment to a nitrogen of a compound or salt of any one of Formulas (I-A), (I-B), (I-C), (I-D), (I-E), (II-A), (II-B), (II-C), and (II-D) and Table 14 and RX represents a reactive moiety. The reactive moiety may be selected, for example, from an electrophile, e.g., an α,β-unsaturated carbonyl, such as a maleimide, and a leaving group. For example, -L³ can be represented by the formulas set forth in Table 4 below:

TABLE 4

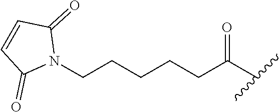 [SMILES: *C(=O)CCCCCN1C(=O)C=CC1=O]

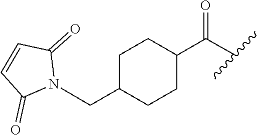 [SMILES: *C(=O)C1CCC(CN2C(=O)C=CC2=O)CC1]

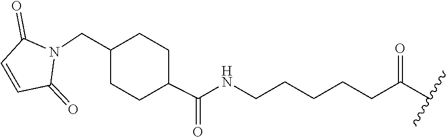 [SMILES: *C(=O)CCCCCNC(=O)C1CCC(CN2C(=O)C=CC2=O)CC1]

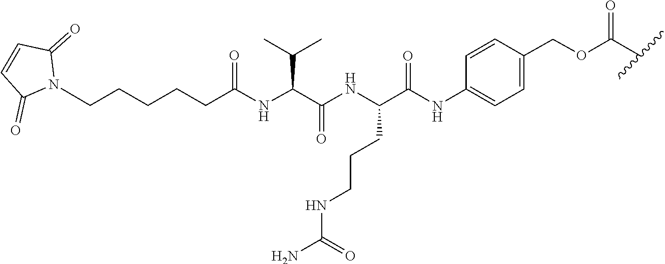 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

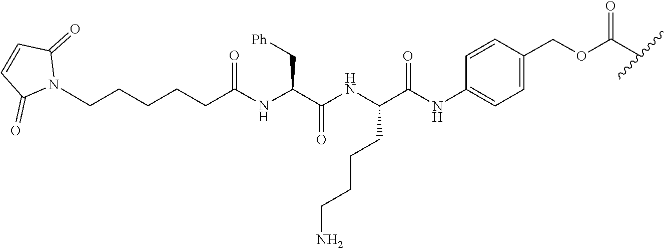 [SMILES: *C(=O)OCc1ccc(NC(=O)[C@H](CCCCN)NC(=O)[C@H](Cc2ccccc2)NC(=O)CCCCCN2C(=O)C=CC2=O)cc1]

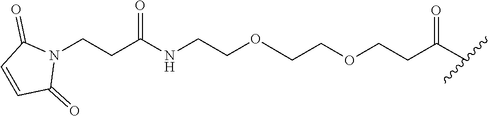 [SMILES: *C(=O)CCOCCOCCNC(=O)CCN1C(=O)C=CC1=O]

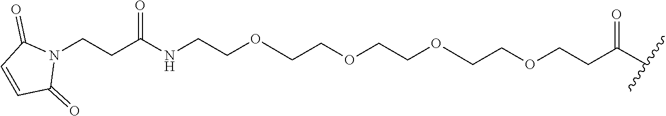 [SMILES: *C(=O)CCOCCOCCOCCOCCNC(=O)CCN1C(=O)C=CC1=O]

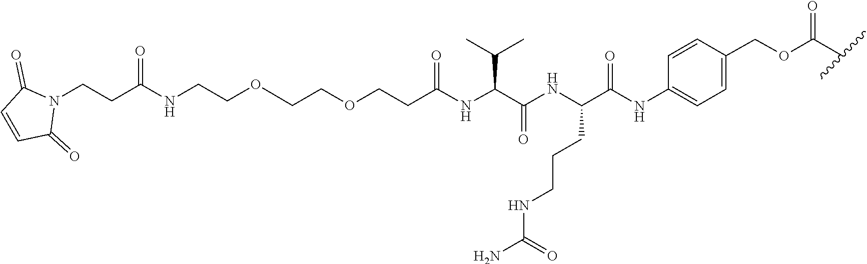 [SMILES: *C(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1]

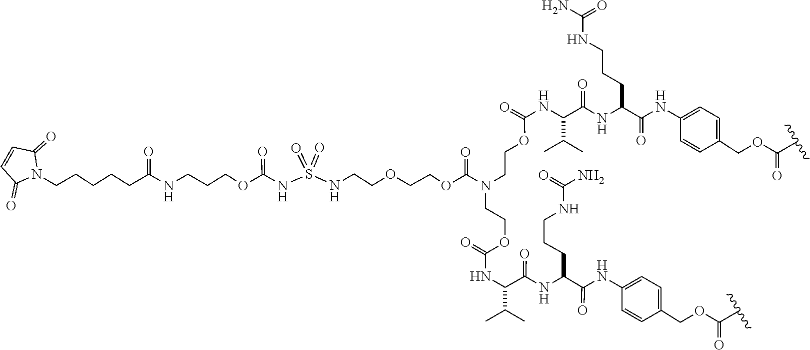 [SMILES: *C(=O)OCc1ccc(NC(=O)[C@H](CCCNC(N)=O)NC(=O)[C@@H](NC(=O)OCCN(CCOC(=O)N[C@H](C(=O)N[C@@H](CCCNC(N)=O)C(=O)Nc2ccc(COC(*)=O)cc2)C(C)C)C(=O)OCCOCCNS(=O)(=O)NC(=O)OCCCNC(=O)CCCCCN2C(=O)C=CC2=O)C(C)C)cc1]

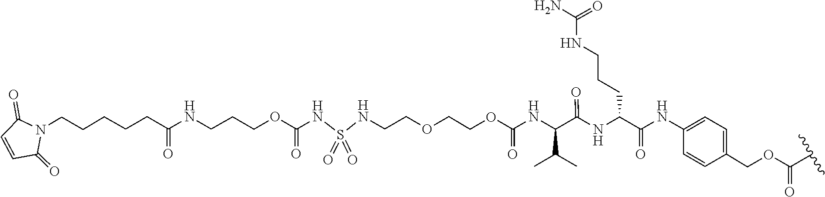 [SMILES: *C(=O)OCc1ccc(NC(=O)[C@@H](CCCNC(N)=O)NC(=O)[C@H](NC(=O)OCCOCCNS(=O)(=O)NC(=O)OCCCNC(=O)CCCCCN2C(=O)C=CC2=O)C(C)C)cc1]

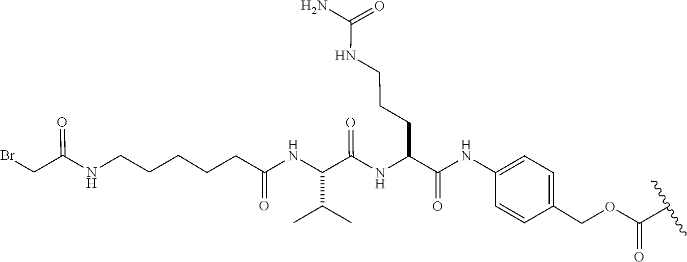 [SMILES: *C(=O)OCc1ccc(NC(=O)[C@H](CCCNC(N)=O)NC(=O)[C@@H](NC(=O)CCCCCNC(=O)CBr)C(C)C)cc1]

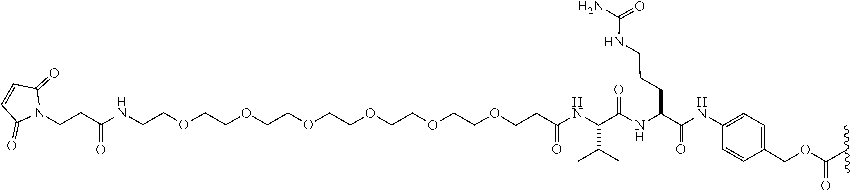 [SMILES: *C(=O)OCc1ccc(NC(=O)[C@H](CCCNC(N)=O)NC(=O)[C@@H](NC(=O)CCOCCOCCOCCOCCOCCOCCNC(=O)CCN2C(=O)C=CC2=O)C(C)C)cc1]

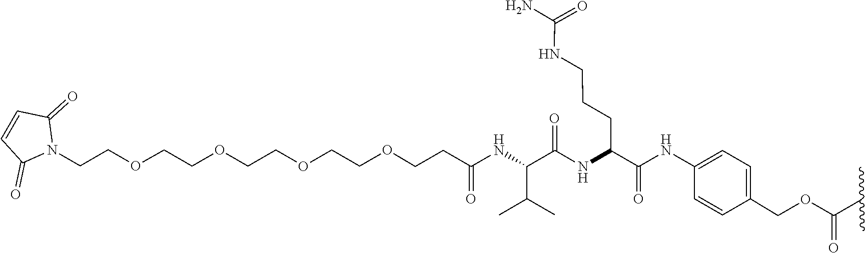 [SMILES: *C(=O)OCc1ccc(NC(=O)[C@H](CCCNC(N)=O)NC(=O)[C@@H](NC(=O)CCOCCOCCOCCOCCN2C(=O)C=CC2=O)C(C)C)cc1]

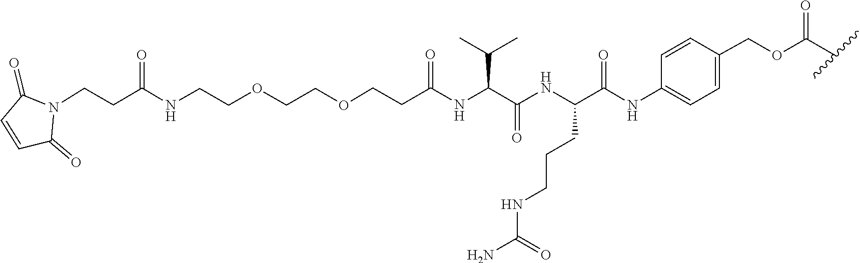 [SMILES: *C(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1]

wherein

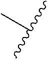

represents attachment to a nitrogen of a compound or salt of any one of Formulas (I-A), (I-B), (I-c), (I-D), (I-E), (II-A), (II-B), (II-C), and (II-ID) and Table 14.

When conjugated to the cysteine residue of the antibody or targeting moiety, such linkers can be, for example, represented by the Formulas set forth in Table 5 below:

TABLE 5

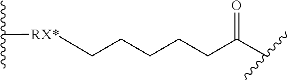

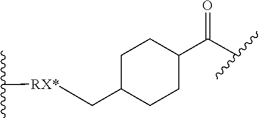

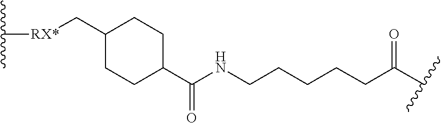

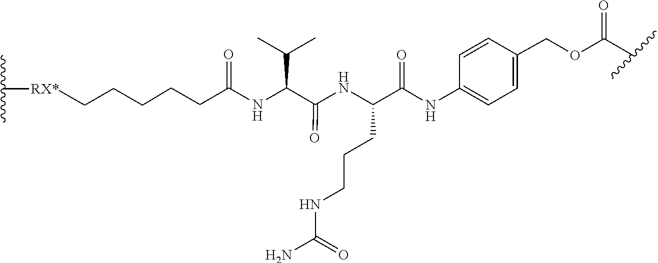

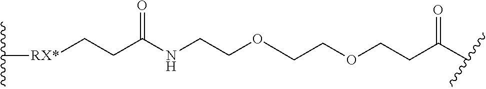

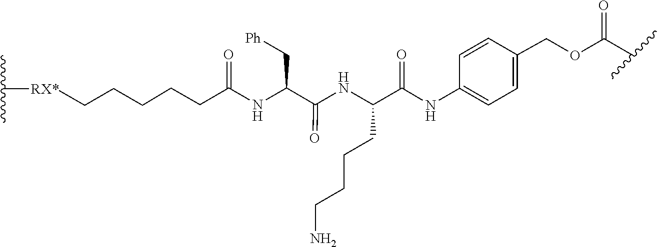

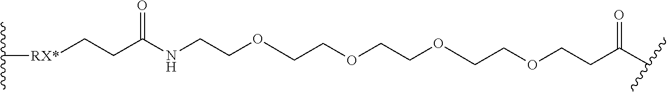

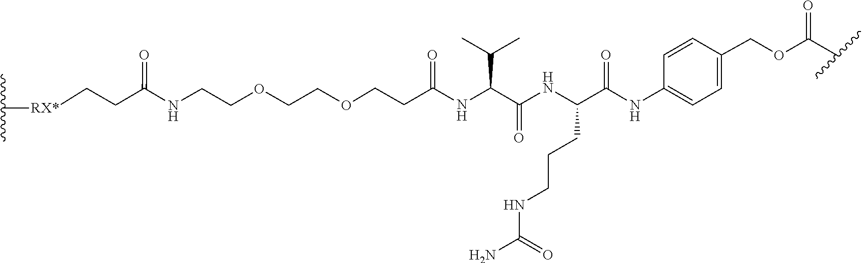

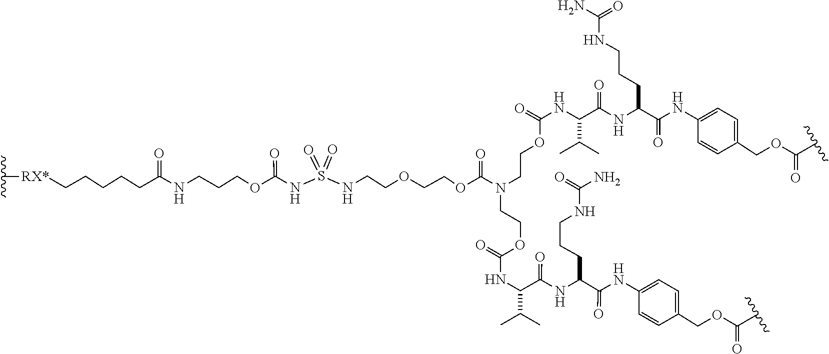

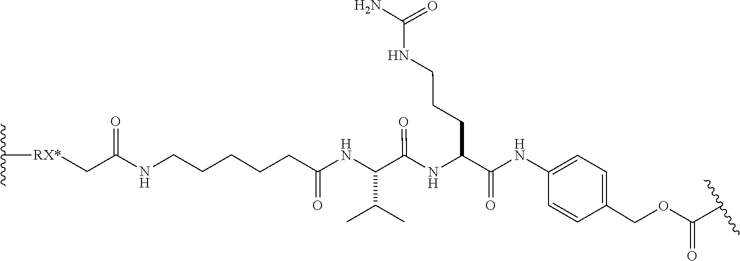

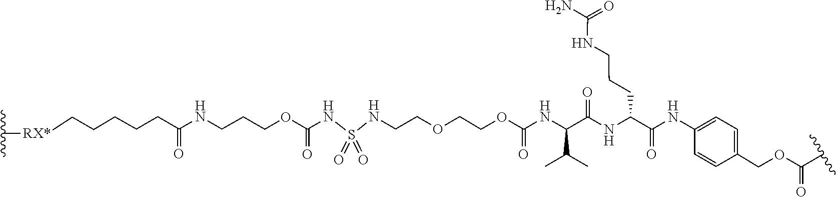

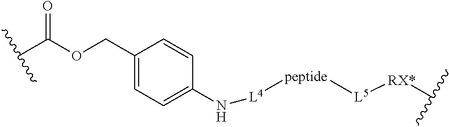

wherein RX* is a bond, a succinimide moiety, or a hydrolyzed succinimide moiety bound to a cysteine residue of the antibody construct, wherein [symbol] on RX* represents the point of attachment to such residue; L⁴ when present represents the C-terminus of the peptide and L⁵ is selected from a bond, alkylene and heteroalkylene, wherein L⁵ is optionally substituted with one or more groups independently selected from R³⁰; and R³⁰ when present is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂. A particularly preferred peptide is val-ala or val-cit.

In some embodiments for a compound or salt of Formula (I-A), (I-B), (I-C), (I-D), (I-E), (II-A), (II-B), (II-C), and (II-D) and Table 14 wherein attachment of the linker is to a nitrogen of a compound or salt of any one of Formulas (I-A), (I-B), (I-C), (I-D), (I-E), (II-A), (II-B), (II-C), and (II-D) and Table 14 and conjugation is to a lysine residue of an antibody or other targeting moiety, -L³ is represented by the formulas set forth in Table 6 below:

TABLE 6

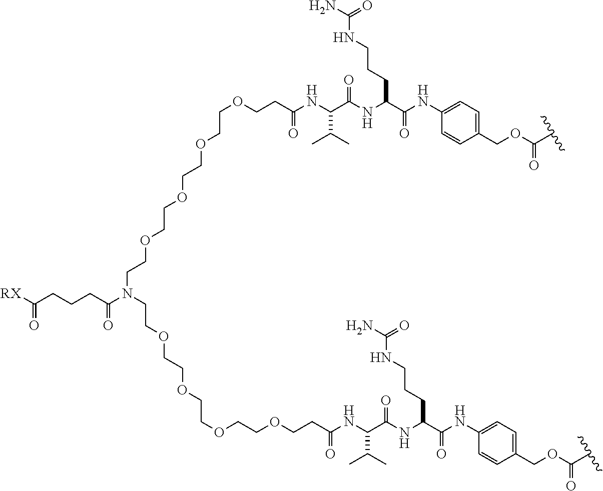

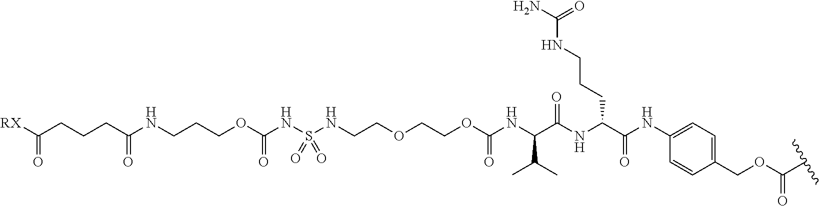

wherein

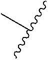

represents attachment to a nitrogen of a compound or salt of any one of Formulas (I-A), (I-B), (I-C), (I-D), (I-E), (II-A), (II-B), (II-C), and (II-D) and Table 14 and RX represents a reactive moiety. The reactive moiety may be selected from activated esters. For example, -L³ can be represented by the formulas set forth in Table 7 below:

TABLE 7

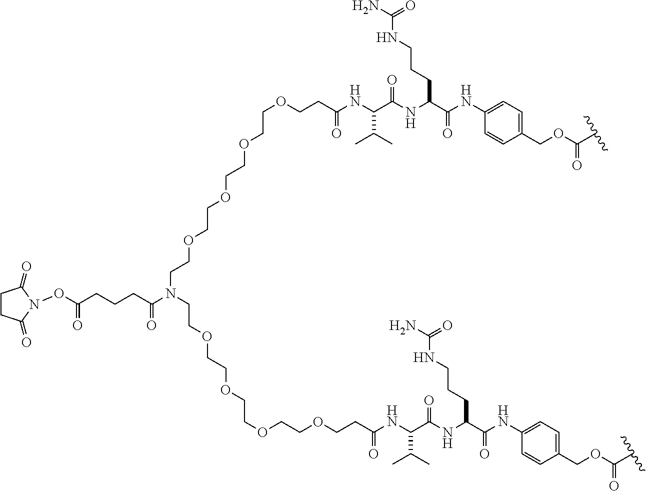 [SMILES: *C(=O)OCc1ccc(NC(=O)[C@H](CCCNC(N)=O)NC(=O)[C@@H](NC(=O)CCOCCOCCOCCOCCN(CCOCCOCCOCCOCCC(=O)N[C@H](C(=O)N[C@@H](CCCNC(N)=O)C(=O)Nc2ccc(COC(*)=O)cc2)C(C)C)C(=O)CCCC(=O)ON2C(=O)CCC2=O)C(C)C)cc1]

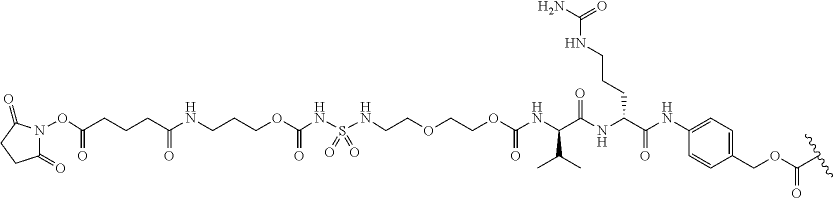 [SMILES: *C(=O)OCc1ccc(NC(=O)[C@@H](CCCNC(N)=O)NC(=O)[C@H](NC(=O)OCCOCCNS(=O)(=O)NC(=O)OCCCNC(=O)CCCC(=O)ON2C(=O)CCC2=O)C(C)C)cc1]

When conjugated to the lysine residue of an antibody or other targeting moiety, such linkers, can, for example, be represented by the Formulas set forth in Table 8 below wherein RX* is a bond to a nitrogen of the lysine residue of the antibody construct or targeting moiety, wherein [symbol] on RX* represents the point of attachment to such residue:

TABLE 8

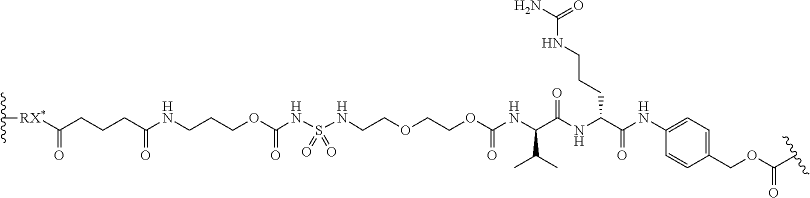

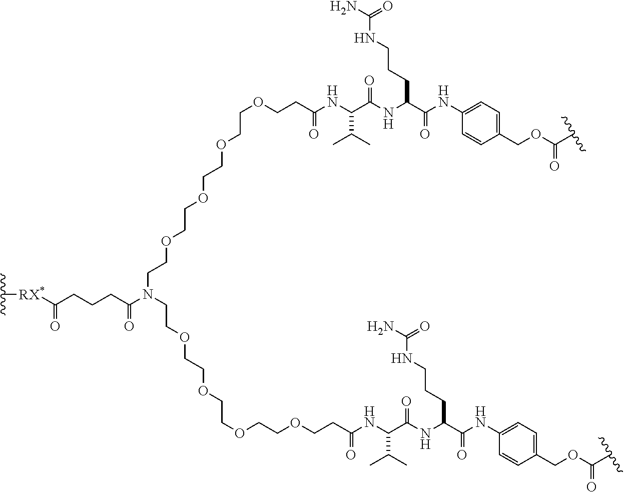

As noted,

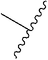

represents attachment to a nitrogen of a compound or salt of any one of Formulas (I-A), (I-B), (I-C), (I-D), (I-E), (II-A), (II-B), (II-C), and (II-D) and Table 14. In exemplary embodiments, the linkers described herein, including those in the preceding paragraphs, are attached to a compound of the present invention through R⁴ of Ring A or Ring B. In some such exemplary embodiments, at least one R⁴ is independently selected from: (i) substituted C₁-C₆alkyl with the proviso that C₁-C₆alkyl is substituted with —NR⁵²R⁵² and at least one of —OR⁵², —CO₂R⁵², —(C₁-C₆alkyl)-OR⁵², or (C₁-C₆alkyl)-CO₂R⁵² and wherein one R⁵² of —NR⁵²R⁵² is replaced with -L³; (ii) —OR⁵⁰ wherein R⁵⁰ of —OR⁵⁰ is substituted C₁-C₆alkyl, at least one substituents on said alkyl is —NR⁵²R⁵² and wherein one R⁵² of —NR⁵²R⁵² is replaced with -L³; (iii) —OR⁵⁰ wherein R⁵⁰ of —OR⁵⁰ is a heterocycle or carbocycle, at least one substituent on said heterocycle and carbocycle is —NR⁵²R⁵², or C₁-C₆alkyl substituted with at least one substituent selected from —NR⁵²R⁵², and wherein one R⁵² of —NR⁵²R⁵² is replaced with -L³; (iv) substituted heterocycle wherein at least one substituent on said heterocycle is —NR⁵²R⁵², or C₁-C₆alkyl substituted with at least one substituent selected from —NR⁵²R⁵², and wherein one R⁵² of —NR⁵²R⁵² is replaced with -L³; (v) —NR⁵¹R⁵¹ wherein one R⁵¹ of —NR⁵¹R¹⁵ is replaced with -L³; (vi) —NR⁵¹R⁵¹ wherein one R⁵¹ of —NR⁵¹R⁵¹ is C₁-C₆alkyl substituted with at least one —NR⁵²R⁵² and wherein one R⁵² of —NR⁵²R⁵² is replaced with -L³; (vii) —NR⁵¹R⁵¹ wherein one R⁵¹ of —NR⁵¹R⁵¹ is a heterocycle and at least one substituent on said heterocycle is —NR⁵²R⁵², or C₁-C₆alkyl substituted with at least one substituent selected from —NR⁵²R⁵², and wherein one R⁵² of —NR⁵²R⁵² is replaced with -L³; (viii) —NR⁵¹R⁵¹ wherein two R⁵¹ of —NR⁵¹R⁵¹ are taken together with the N atom to which they are attached, they form a 5 or 6 membered unsubstituted or substituted saturated N-containing heterocycle wherein at least one of said substituents is NR⁵²R⁵² or C₁-C₆alkyl substituted with at least one substituent selected from —NR⁵²R⁵² and wherein one R⁵² of —NR⁵²R⁵² is replaced with -L³; or (ix) two R⁴ on adjacent atoms are taken together with the atoms to which they are attached to form an unsubstituted or substituted 5 or 6 membered monocyclic carbocycle or unsubstituted or substituted 6 membered monocyclic heterocycle with one or two ring heteroatoms selected from oxygen and nitrogen and at least one substituent on said carbocycle and heterocycle is NR⁵²R⁵² or C₁-C₆alkyl substituted with at least one substituent selected from —NR⁵²R⁵² and wherein one R⁵² of —NR⁵²R⁵² is replaced with -L³.

R⁴ can be, for example, selected from any of the groups set forth in Table 9A wherein R⁵¹ and R⁵² are as described herein (including hydrogen or C₁₋₃ alkyl (e.g., methyl)) and the wavy line indicates attachment to ring A or ring B:

TABLE 9A

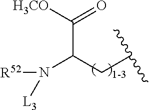

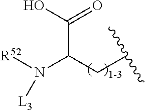

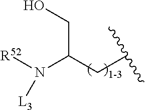

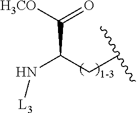

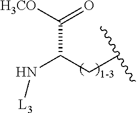

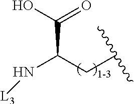

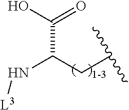

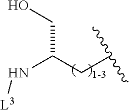

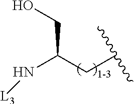

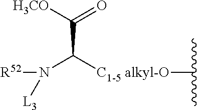

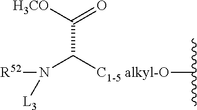

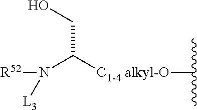

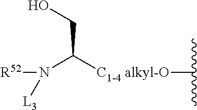

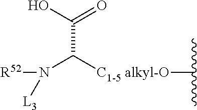

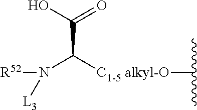

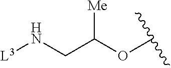

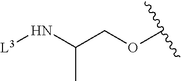

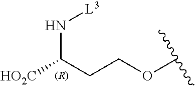

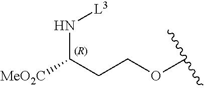

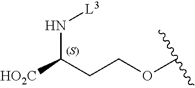

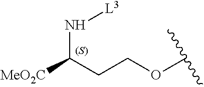

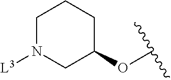

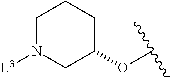

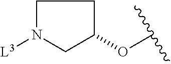

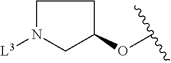

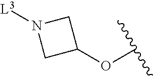

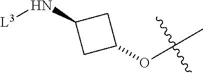

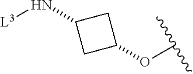

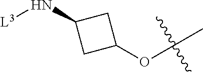

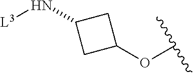

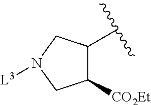

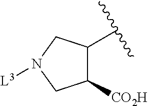

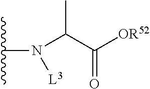

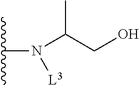

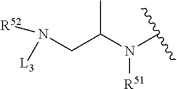

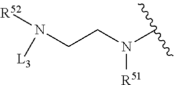

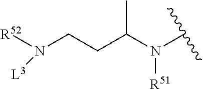

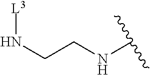

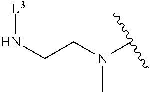

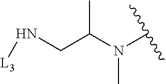

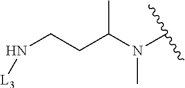

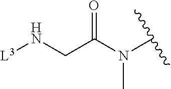

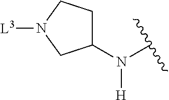

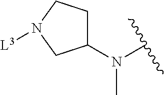

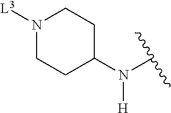

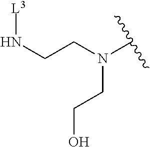

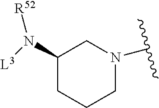

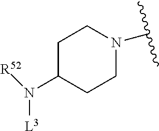

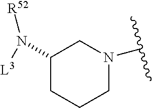

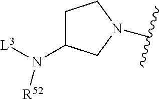

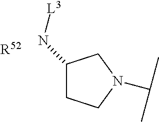

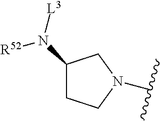

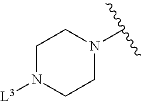

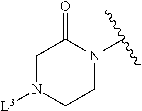

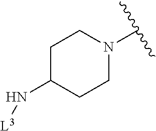

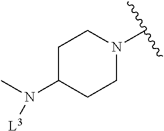

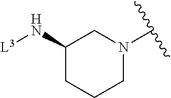

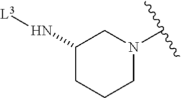

In exemplary embodiments, the linkers described herein, including those in the preceding paragraphs, are attached to a compound of the present invention at a nitrogen atom as shown below in Table 9B wherein L³ represents the linker:

TABLE 9B

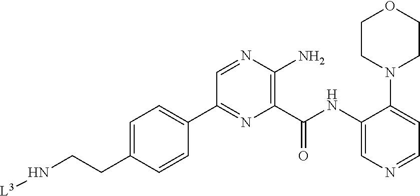

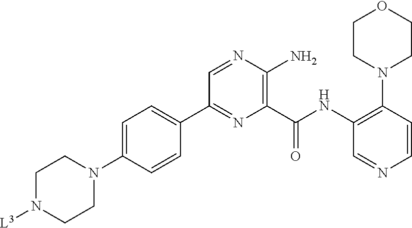

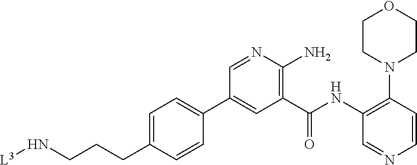

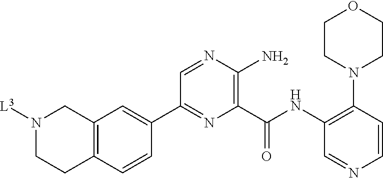

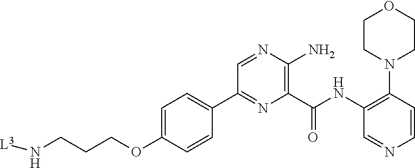

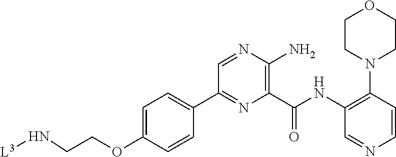

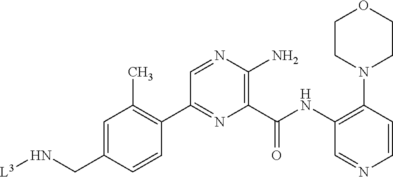

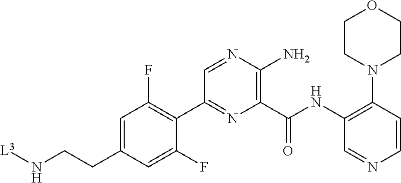

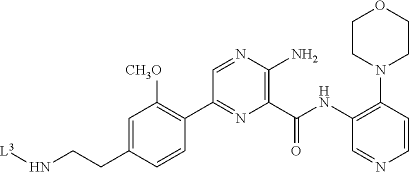

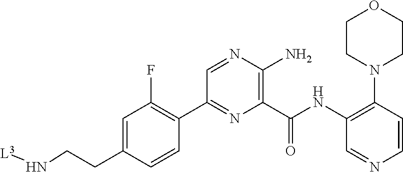

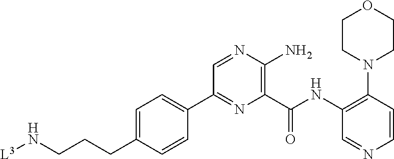

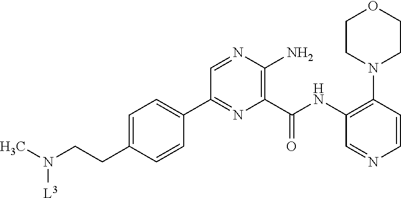

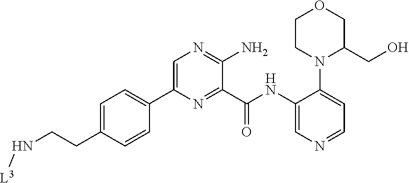

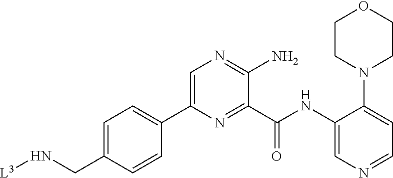

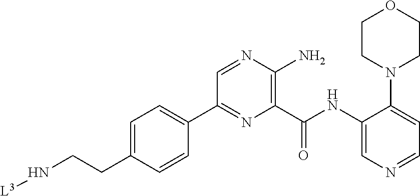

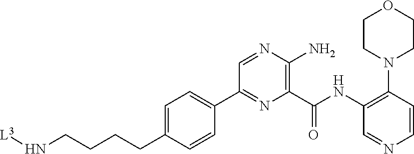

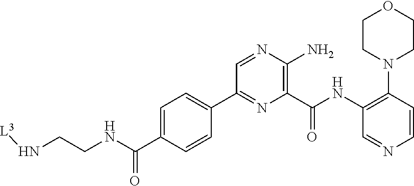

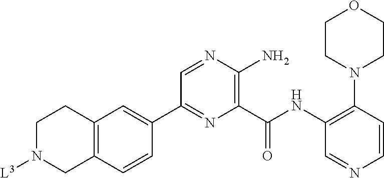

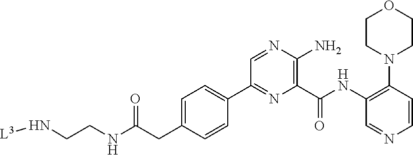

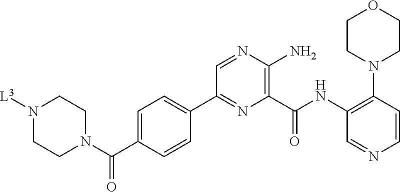

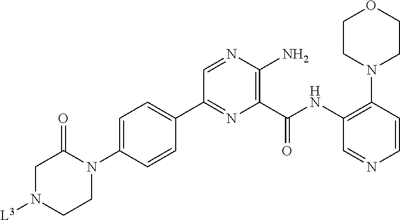

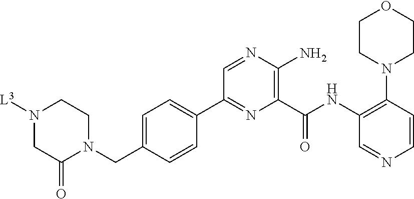

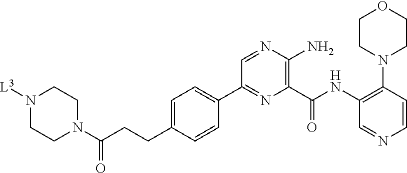

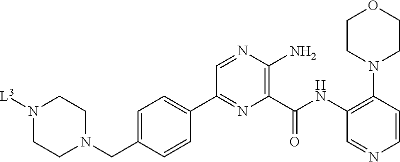

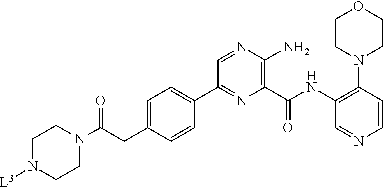

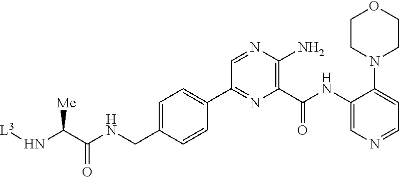

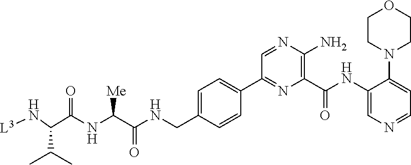

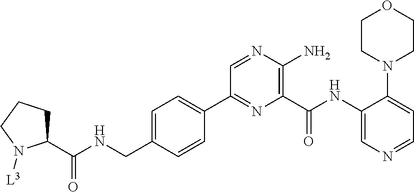

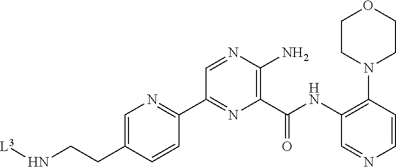

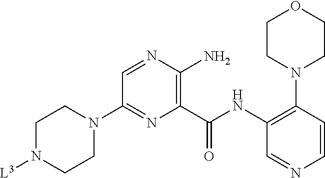

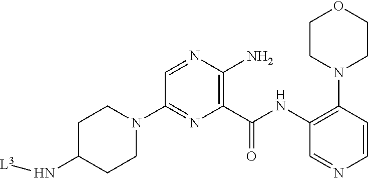

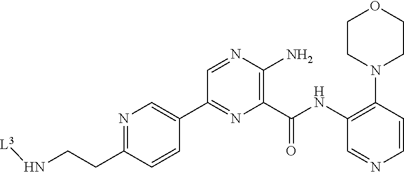

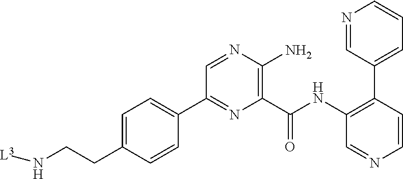

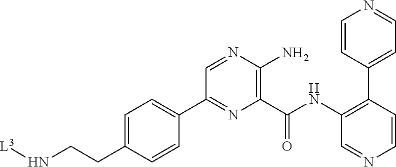

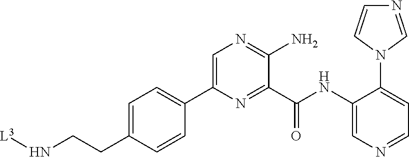

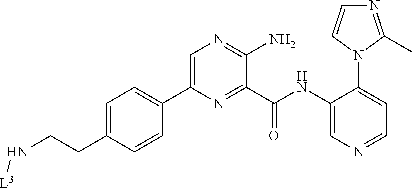

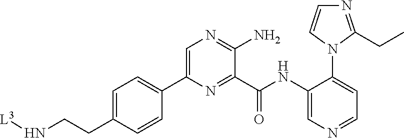

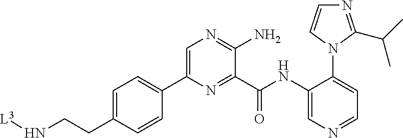

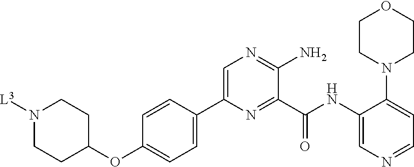

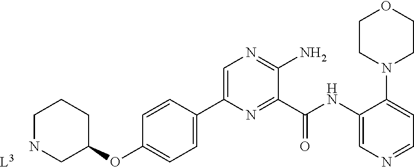

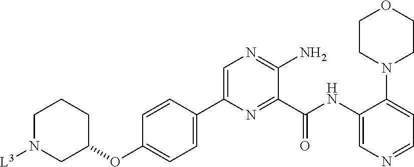

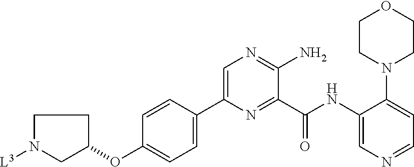

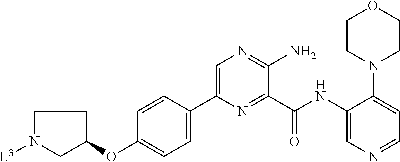

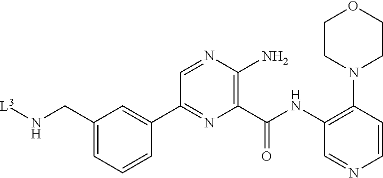

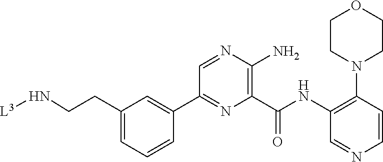

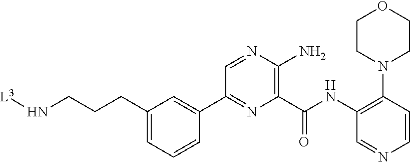

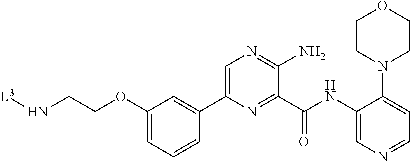

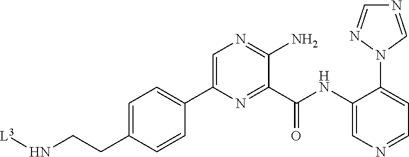

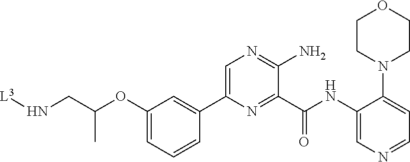

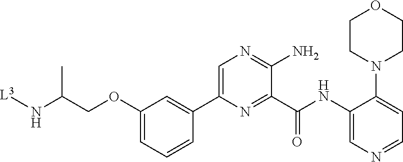

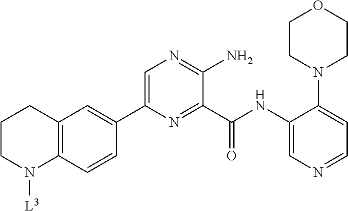

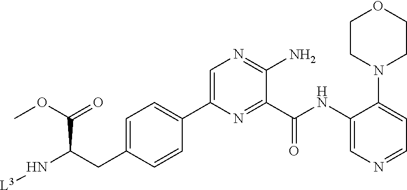

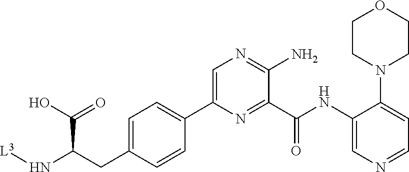

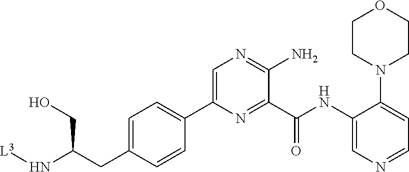

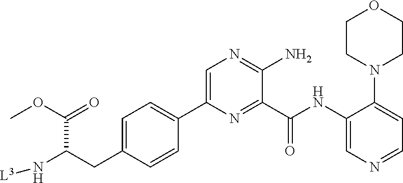

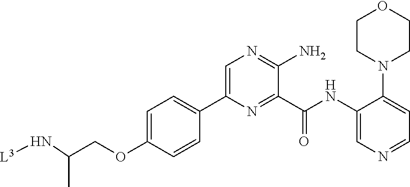

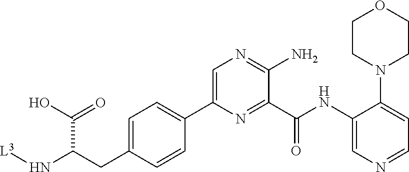

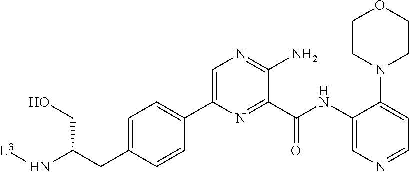

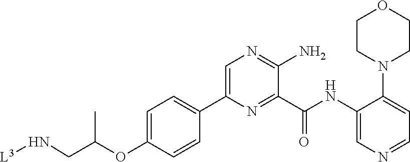

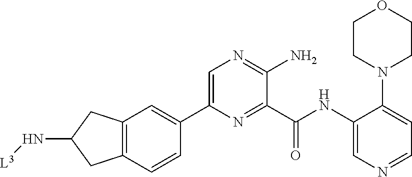

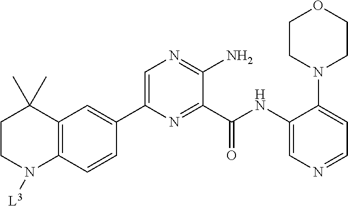

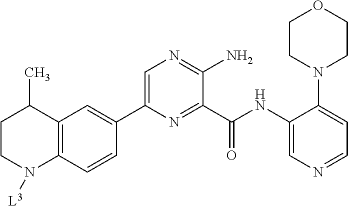

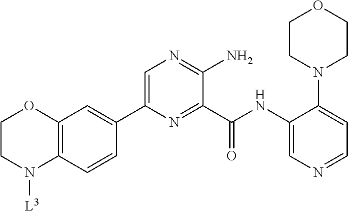

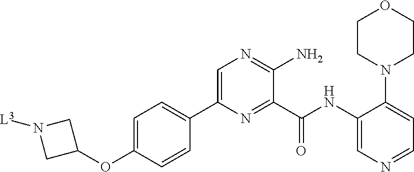

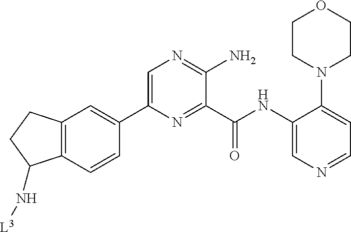

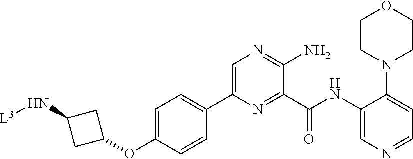

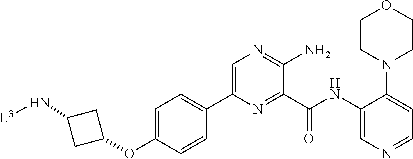

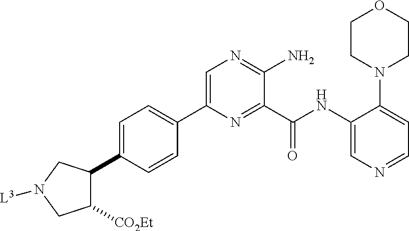

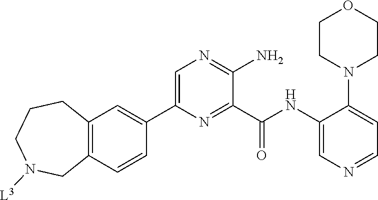

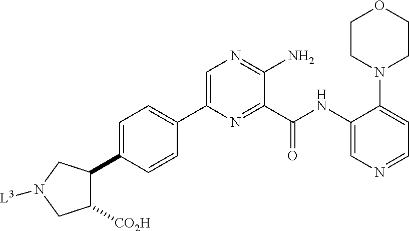

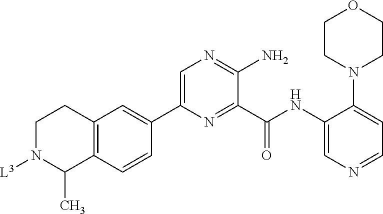

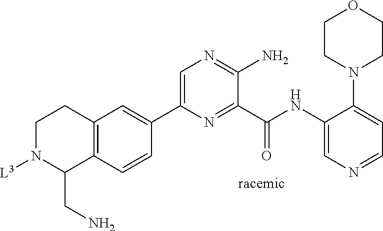

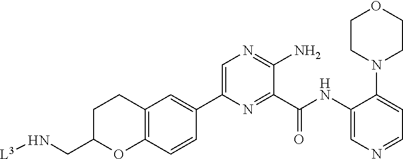

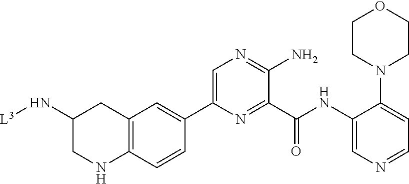

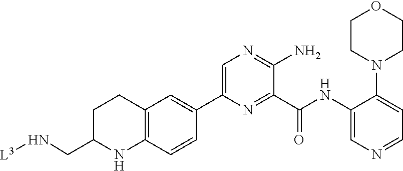

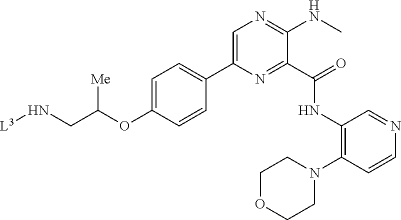

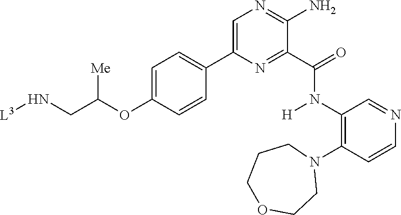

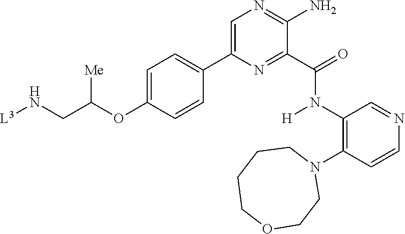

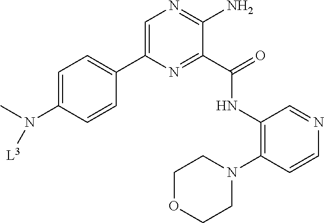

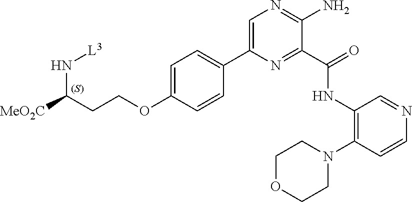

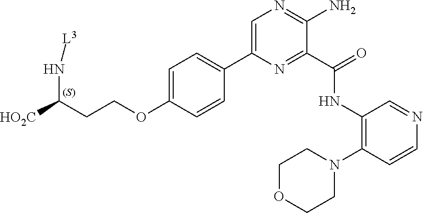

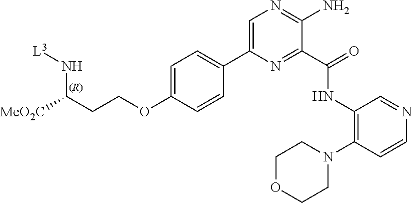

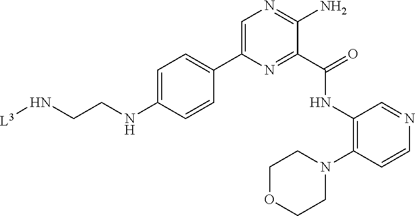

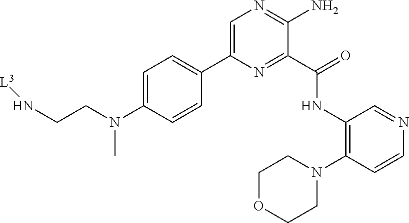

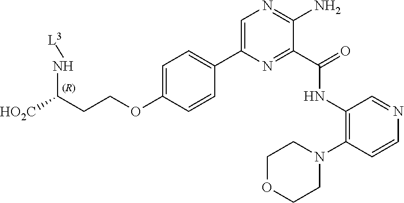

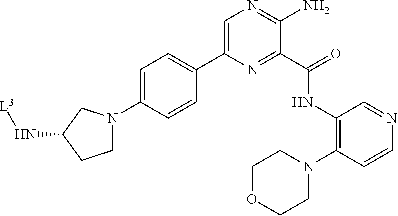

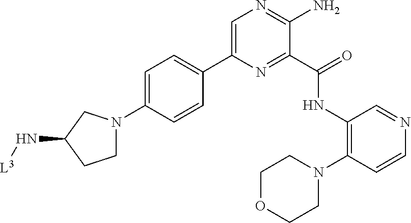

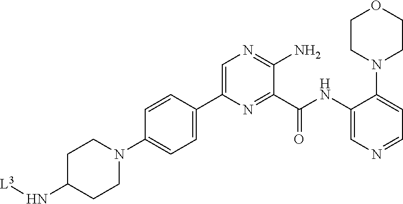

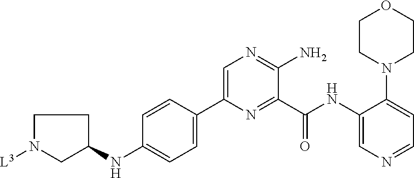

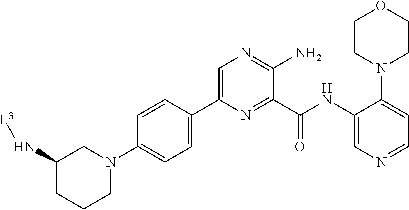

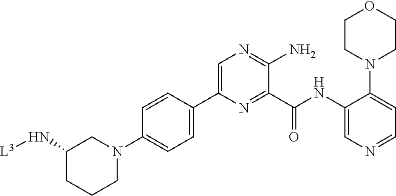

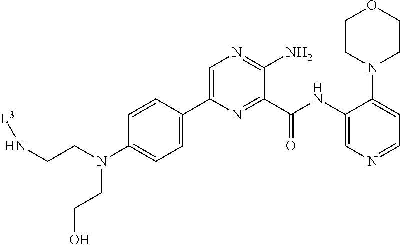

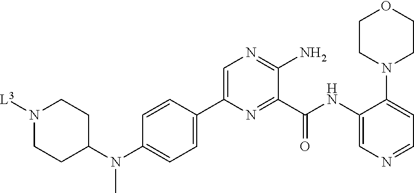

In some embodiments for a compound or salt of Formula (I-A), (I-B), (I-C), (I-D), (I-E), (II-A), (II-B), (II-C), and (II-D) wherein attachment of the linker is to a sulfur of a compound or salt of any one of Formulas (I-A), (I-B), (I-C), (I-D), (I-E), (II-A), (II-IB), (II-C), and (II-ID) and Table 14 and conjugation is to a lysine residue of an antibody or other targeting moiety, -L³ is represented by the formula set forth below in Table 10:

TABLE 10

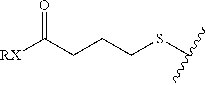

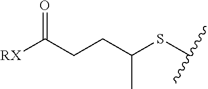

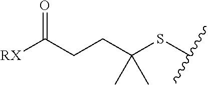

wherein

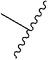

represents attachment to a sulfur of a compound or salt of any one of Formulas (I-A), (I-B), (I-C), (I-D), (I-E), (II-A), (II-B), (II-C), and (II-D) and Table 14 and RX represents a reactive moiety. The reactive moiety may be selected from an activated ester. For example, -L³ can be represented by the formulas:

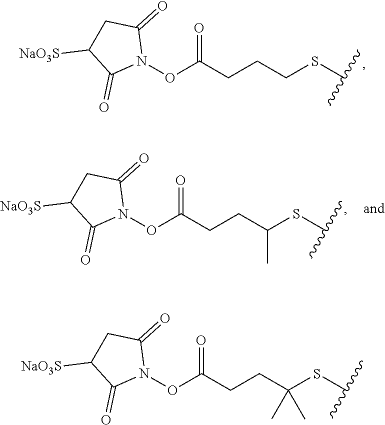

When conjugated to the lysine residue of an antibody or other targeting moiety, such linkers, can be represented by the following Formulas in Table 11:

TABLE 11

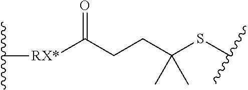

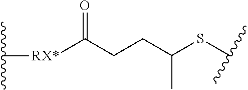

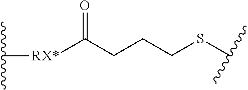

wherein RX* is a bound to a nitrogen of the lysine residue of the antibody construct or targeting moiety, wherein [symbol] on RX* represents the point of attachment to such residue:

As noted,

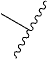

represents attachment to a sulfur atom of a compound or salt of any one of Formulas (I-A), (I-B), (I-C), (I-D), (I-E), (II-A), (II-B), (II-C), and (II-D) and Table 14. In exemplary embodiments, the linkers described herein, including those in the preceding paragraphs, are attached at a sulfur atom to a compound or salt of as shown below in Table 12 wherein L³ represents the linker:

TABLE 12

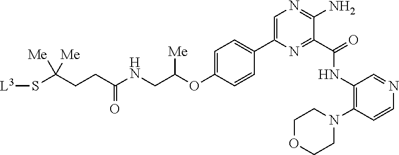

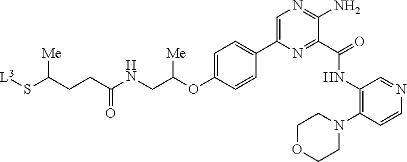

In other exemplary embodiments, exemplary linkers are attached at an oxygen atom of a compound or salt as shown below in Table 13 wherein L³ represents the linker:

TABLE 13

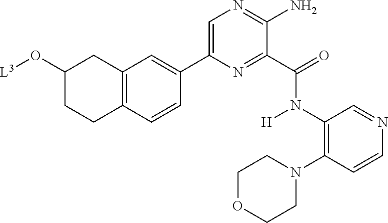

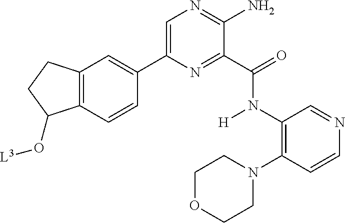

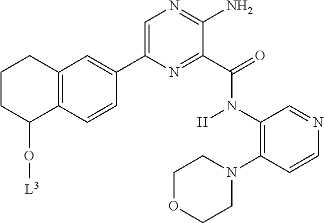

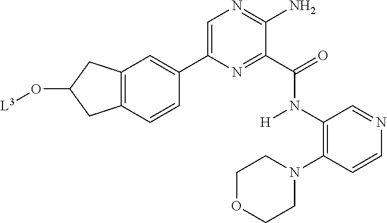

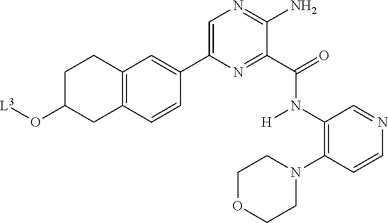

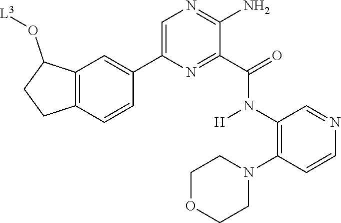

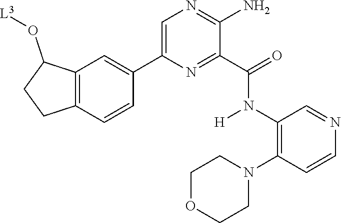

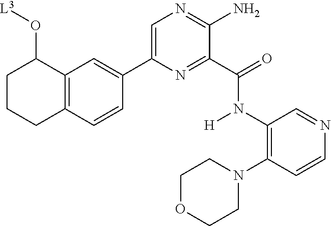

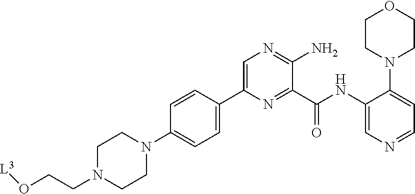

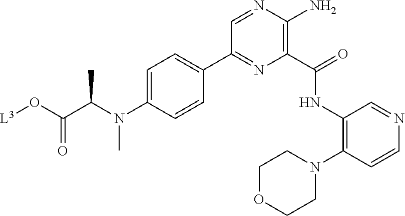

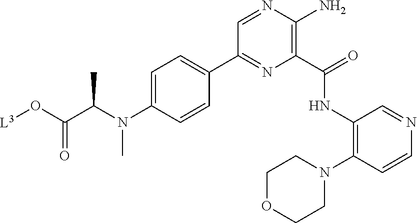

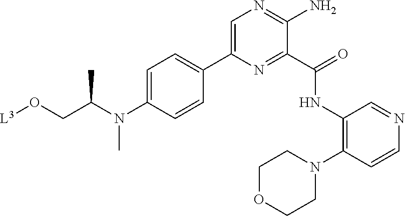

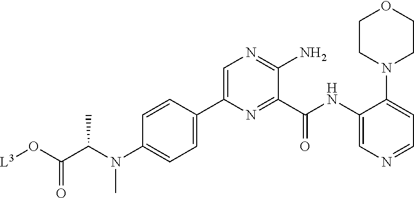

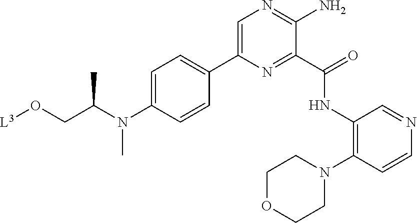

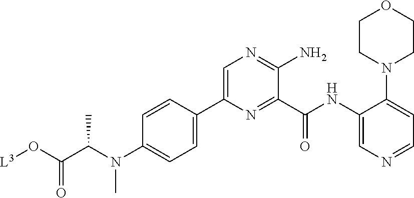

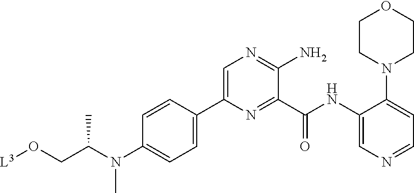

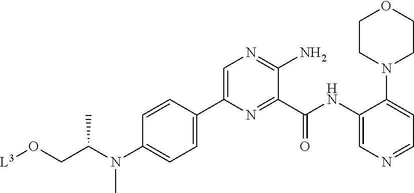

As is known by skilled artisans, the linker selected for a particular conjugate may be influenced by a variety of factors, including but not limited to, the site of attachment to the antibody construct (e.g., lys, cys or other amino acid residues), structural constraints of the drug pharmacophore and the lipophilicity of the drug. The specific linker selected for a conjugate should seek to balance these different factors for the specific antibody construct/drug combination.

The properties of the linker, or linker-compound, may also impact aggregation of the conjugate under conditions of use and/or storage. Typically, conjugates reported in the literature contain no more than 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where the payload compound is more hydrophobic in nature, it may be desirable to select linkers that are relatively hydrophilic as a means of reducing conjugate aggregation, especially in instances where DARs greater than 3-4 are desired. Thus, in certain embodiments, the linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In particular embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

Exemplary Linker-Compounds of the present invention include those set forth in Tables 15, 16, and 17, and salts thereof (including pharmaceutically acceptable salts thereof.

### Conjugates of PROTACS

In certain embodiments, a conjugate of a compound described herein can be designed to increase ubiquitin-mediated target protein destruction via the ubiquitin pathway. The process of attaching ubiquitin molecules to a protein target typically involves 3 enzymes and steps: 1) an E1 enzyme that can activate ubiquitin, 2) an E2 enzyme that can transfer activated ubiquitin, and 3) a multi-subunit E3 enzyme ligase that can receive the activated ubiquitin and catalyze a ubiquitin attachment to the target protein.

In some embodiments, a conjugate includes a proteolysis targeting module (PTM; also referred to as a proteolysis-targeting chimera or PROTAC). A PTM can comprise a small molecule that can bind to an E3 ubiquitin ligase subunit and a target binding moiety (a compound described herein) that binds a protein target. The E3 ubiquitin ligase binding small molecule is attached, directly or by a spacer (S), to the target binding moiety.

### Pharmaceutical Formulations

The compositions and methods described herein may be considered useful as pharmaceutical compositions for administration to a subject in need thereof. Pharmaceutical compositions may comprise at least the compositions described herein and one or more pharmaceutically acceptable carriers, diluents, excipients, stabilizers, dispersing agents, suspending agents, and/or thickening agents. The composition may comprise the conjugate having an antibody construct and an amino-pyrazinecarboxamide compound. The composition may comprise the conjugate having an antibody construct and an amino-pyrazinecarboxamide compound. The composition may comprise the conjugate having an antibody construct, a target binding domain, and an amino-pyrazinecarboxamide compound. The composition may comprise any conjugate described herein. In some embodiments, the antibody construct is an anti-LRRC15 antibody. A conjugate may comprise an anti-LRRC15 antibody and an amino-pyrazinecarboxamide compound. In some embodiments, the antibody construct is an anti-ASGR1 antibody. A conjugate may comprise an anti-ASGR1 antibody and an amino-pyrazinecarboxamide compound. A pharmaceutical composition can comprise at least the compounds, salts or conjugates described herein and one or more of buffers, antibiotics, steroids, carbohydrates, drugs (e.g., chemotherapy drugs), radiation, polypeptides, chelators, adjuvants and/or preservatives.

Pharmaceutical compositions may be formulated using one or more physiologically-acceptable carriers comprising excipients and auxiliaries. Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate. The pharmaceutical compositions may also include the compounds, salts or conjugates in a free-base form or pharmaceutically-acceptable salt form.

Methods for formulation of the conjugates may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for re-constitution with a suitable vehicle, e.g., sterile pyrogen-free water, before use.

Pharmaceutical compositions of the conjugates may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as often further may comprise more than one active compound (e.g., a compound, salt or conjugate and other agents) as necessary for the particular indication being treated. The active compounds may have complementary activities that do not adversely affect each other. For example, the composition may comprise a chemotherapeutic agent, cytotoxic agent, cytokine, growth-inhibitory agent, anti-hormonal agent, anti-angiogenic agent, and/or cardioprotectant. Such molecules may be present in combination in amounts that are effective for the purpose intended.

The compositions and formulations may be sterilized. Sterilization may be accomplished by filtration through sterile filtration.

The compositions may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, the compounds, salts or conjugates may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

Sustained-release preparations may be also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, maniose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Pharmaceutical formulations of the conjugates may have an average drug-antibody construct ratio (“DAR”) selected from about 1 to about 20 or from about 1 to about 10, wherein the drug is a compound or salt of any one of Formulas (I-A), (I-B), (I-C), (I-D), (I-E), (II-A), (II-B), (II-C), and (II-D). In certain embodiments, the average DAR of the formulation is from about 2 to about 8, such as from about 3 to about 8, such as from about 3 to about 7, such as about 3 to about 5 or such as about 2. In certain embodiments, a pharmaceutical formulation has an average DAR of about 3, about 3.5, about 4, about 4.5 or about 5.

### Therapeutic Applications

The compositions, conjugates and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compositions, conjugates and methods can be useful as a therapeutic, for example, a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the compositions, and conjugates can be administered to a subject in need thereof, often for treating and/or preventing a condition or progression thereof. A pharmaceutical composition can affect the physiology of the subject, such as the immune system, an inflammatory response, or other physiologic affect. A therapeutically-effective amount can vary depending on the severity of the disease, the age and relative health of the subject, the potency of the compounds used, and other factors.

Treat and/or treating refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual. Preventing can also refer to preventing re-occurrence of a disease or condition in a patient that has previously been treated for the disease or condition, e.g., by preventing relapse.

A therapeutically effective amount (also referred to as an effective amount) can be the amount of a composition (e.g., conjugate or compound) or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques and the teachings provided herein.

The conjugates that can be used in therapy can be formulated and dosages established in a fashion consistent with good medical practice taking into account the disease or condition to be treated, the condition of the individual patient, the site of delivery of the composition, the method of administration and other factors known to practitioners. The compositions can be prepared according to the description of preparation described herein.

Pharmaceutical compositions can be used in the methods described herein and can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition to a subject in need thereof depends on several factors including, for example but not limited to, the health of the subject, the specific disease or condition of the patient, the grade or level of a specific disease or condition of the patient, the additional treatments the subject is receiving or has received, and the like.

The methods and compositions can be for administration to a subject in need thereof. Often, administration of the compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition can be administered to a subject by additional routes of administration, for example, by inhalation, oral, dermal, intranasal, or intrathecal administration.

Compositions and conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The administrations can be weekly, biweekly (every two weeks), every three weeks, monthly or bimonthly.

The compositions, conjugates and methods provided herein may be useful for the treatment of a plurality of diseases, conditions, preventing a disease or a condition in a subject or other therapeutic applications for subjects in need thereof. Often the compositions, conjugates and methods provided herein may be useful for treatment of hyperplastic conditions, including but not limited to, neoplasms, cancers, tumors and the like. The compositions, conjugates and methods provided herein may be useful in specifically targeting TGFβ1, TGFβR1, TGFβR2, or combinations thereof. The compositions and methods provided herein may be useful in inhibiting TGFβ1, TGFβR1, TGFβR2, or combinations thereof. In one embodiment, the compounds of the present disclosure activate or enhane an immune response. In another embodiment, the conjugates of the present disclosure activate or enhance an immune response.

A condition, such as a cancer, may be associated with expression of a molecule on the cancer cells. Often, the molecule expressed by the cancer cells may comprise an extracellular portion capable of recognition by the antibody construct of the conjugate. A molecule expressed by the cancer cells may be a tumor antigen. An antibody construct of the conjugate may recognize a tumor antigen.

In certain embodiments, the antigen binding domain specifically binds to an antigen that is at least 80% identical to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen that is at least 80% identical to an antigen on a T cell, an APC, and/or a B cell. In certain embodiments, the antigen binding domain may specifically bind to an antigen that is at least 80% identical to an antigen selected from the group consisting of CLTA4, PD-1, OX40, LAG-3, GITR, GARP, CD25, CD27, PD-L1, TNFR2, ICOS, 41BB, CD70, CD73, CD38, or VTCN1. In certain embodiments, the antigen binding domain specifically binds to an antigen that is at least 80% identical to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen that is at least 80% identical to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11 and F2RL2, In certain embodiments, the antigen binding domain may specifically bind to an antigen that is at least 80% identical to an antigen selected from the group consisting of FAP, ADAM12, LRRC15, MMP14, PDPN, CDH11 and F2RL2, In certain embodiments, the antigen binding domain specifically binds to an antigen that is at least 80% identical to an antigen on a tumor cell, a tumor antigen. In certain embodiments, the antigen binding domain specifically binds to an antigen that is at least 80% identical to an antigen selected from the group consisting of MUC16, UPK1B, VTCN1, TMPRSS3, TMEM238, Clorf186, TMPRSS4, CLDN6, CLDN8, STRA6, MSLN or CD73.

In certain embodiments, the antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis. In certain embodiments, the antigen binding domain specifically binds to an antigen on a T cell, an APC, and/or a B cell. In certain embodiments, the antigen binding domain may specifically bind to an antigen selected from the group consisting of CLTA4, PD-1, OX40, LAG-3, GITR, GARP, CD25, CD27, PD-L1, TNFR2, ICOS, 41BB, CD70, CD73, CD38 or VTCN1. In certain embodiments, the antigen binding domain specifically binds to an antigen on a stellate cell, an endothelial cell, a fibroblast cell, a fibrocyte cell, or a cell associated with the pathogenesis of fibrosis or cancer. In certain embodiments, the antigen binding domain may specifically bind to an antigen selected from the group consisting of, PDGFRβ, integrin αvβ1, integrin αvβ3, integrin αvβ6, integrin αvβ8, Endosialin, FAP, ADAM12, LRRC15, MMP14, PDPN, CDHI1 and F2RL2. In certain embodiments, the antigen binding domain may specifically bind to an antigen selected from the group consisting of FAP, ADAM12, LRRC15, MMP14, PDPN, CDH11 and F2RL2. In certain embodiments, the antigen binding domain specifically binds to an antigen on a tumor cell, a tumor antigen. In certain embodiments, the antigen binding domain specifically binds to an antigen selected from the group consisting of MUC16, UPKiB, VTCN1, TMPRSS3, TMEM238, Clorf186, TMPRSS4, CLDN6, CLDN8, STRA6, MSLN or CD73.

Additionally, such antigens may be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers may include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenstrom macroglobulinemia; Wilms tumor and any combination thereof.

Non-limiting examples of fibrosis or fibrotic diseases include adhesive capsulitis, arterial stiffness, arthrofibrosis, atrial fibrosis, cirrhosis, Crohn's disease, collagenous fibroma, cystic fibrosis, Desmoid-type fibromatosis, Dupuytren's contracture, elastofibroma, endomyocardial fibrosis, fibroma of tendon sheath, glial scar, idiopathic pulmonary fibrosis, keloid, mediastinal fibrosis, myelofibrosis, nuchal fibroma, nephrogenic systemic fibrosis, old myocardial infarction, Peyronie's disease, pulmonary fibrosis, progressive massive fibrosis, nonalcoholic steatohepatitis (otherwise known as NASH), radiation-induced lung injury, retroperitoneal fibrosis, scar, scleroderma/systemic sclerosis.

The invention provides any therapeutic compound or conjugate disclosed herein for use in a method of treatment of the human or animal body by therapy. Therapy may be by any mechanism disclosed herein, such as by stimulation of the immune system. The invention provides any therapeutic compound or conjugate disclosed herein for use in stimulation of the immune system, vaccination or immunotherapy, including for example enhancing an immune response. The invention further provides any therapeutic compound or conjugate disclosed herein for prevention or treatment of any condition disclosed herein, for example cancer, autoimmune disease, inflammation, sepsis, allergy, asthma, graft rejection, graft-versus-host disease, immunodeficiency or infectious disease (typically caused by an infectious pathogen).

The invention also provides any therapeutic compound or conjugate disclosed herein for obtaining any clinical outcome disclosed herein for any condition disclosed herein, such as reducing tumour cells in vivo. The invention also provides use of any therapeutic compound or conjugate disclosed herein in the manufacture of a medicament for preventing or treating any condition disclosed herein.

### EXAMPLES

### List of Abbreviations

As used above, and throughout the description of the invention, the following abbreviations, unless otherwise indicated, shall be understood to have the following meanings:
ACN or MeCN acetonitrile
Bn benzyl
BOC or Boc tert-butyl carbamate
CDI 1,1′-carbonyldiimidazole
Cy cyclohexyl
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
equiv equivalent(s)
Et ethyl
EtOH ethanol
EtOAc ethyl acetate
h hour(s)
HATU 1-[bis(dimethylamino)methylene]-1H-1,2,3-triazolo[4,5-b]pyridinium 3-oxid hexafluorophosphate
HFIP 1,1,1,3,3,3-hexafluoropropan-2-ol
HPLC high performance liquid chromatography
LAH lithium aluminum hydride
LCMS liquid chromatography-mass spectrometry
mc-Val-Cit-PAB-PNP [4-[[(2S)-5-(carbamoylamino)-2-[[(2S)-2-[6-(2,5-dioxopyrrol-1-yl)hexanoylamino]-3-methylbutanoyl]amino]pentanoyl]amino]phenyl]methyl (4-nitrophenyl) carbonate
Me methyl
MeOH methanol
MS mass spectroscopy
NMM N-methylmorpholine
NMR nuclear magnetic resonance
PdCl₂(dppf) [1,1′-Bis(diphenylphosphino)ferrocene]dichloropalladium(II)
Pd(OH)₂ palladium hydroxide
PMB para-methoxybenzyl
rt room temperature
TEA triethylamine
TFA trifluoroacetic acid
THF tetrahydrofuran
TLC thin layer chromatography

### General Synthetic Schemes and Examples

The following synthetic schemes are provided for purposes of illustration, not limitation.

The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants (J) are reported in Hertz. For proton spectra the solvent peak was used as the reference peak.

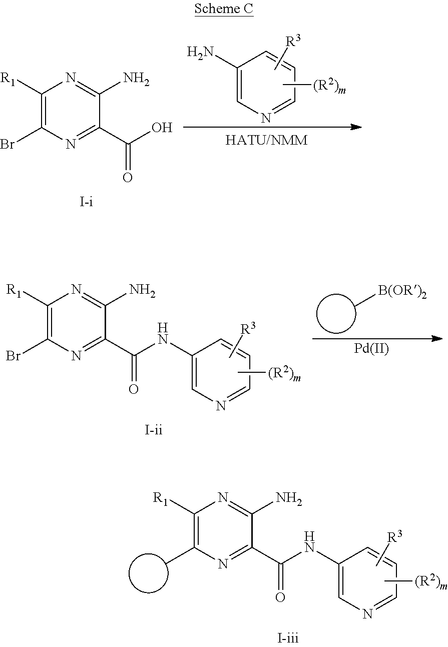

The preparation of compounds are described in the literature (Tebben et al. Acta Cryst. (2016). D72, 658-674; and Berg et al. J Med. Chem. (2012), 55(21), 9107-9119).

2-Amino-5-bromopyrazine carboxylic acids are mixed with 3-aminopyridines in a polar solvent (e.g. DMF) containing a tertiary amine base (e.g. N-methylmorpholine) to form intermediates I-ii. Intermediates I-ii can be mixed with a boronic acid or boronate ester in a solvent such as dioxane and a base (e.g. Na₂CO₃) with a palladium catalyst such as PdCl₂(dppf) at elevated temperature to provide final products (I-iii).

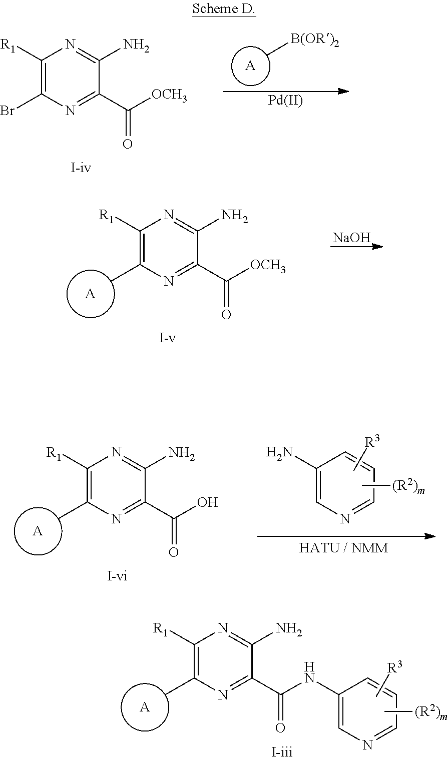

Alternatively, a bromopyrazine (I-iv) can be heated with a boronic acid or boronate ester in a solvent such as dioxane and a base (e.g. Na₂CO₃) with a palladium catalyst such as PdCl₂(dppf) to provide intermediates (I-v). The carboxylic ester can be converted to the carboxylic acid on treatment with a hydroxide base such as NaOH. Intermediates (I-vi) can be coupled to substituted aminopyridines in a polar solvent (e.g. DMF) containing a tertiary amine base (e.g. N-methylmorpholine) to form intermediates I-iii.

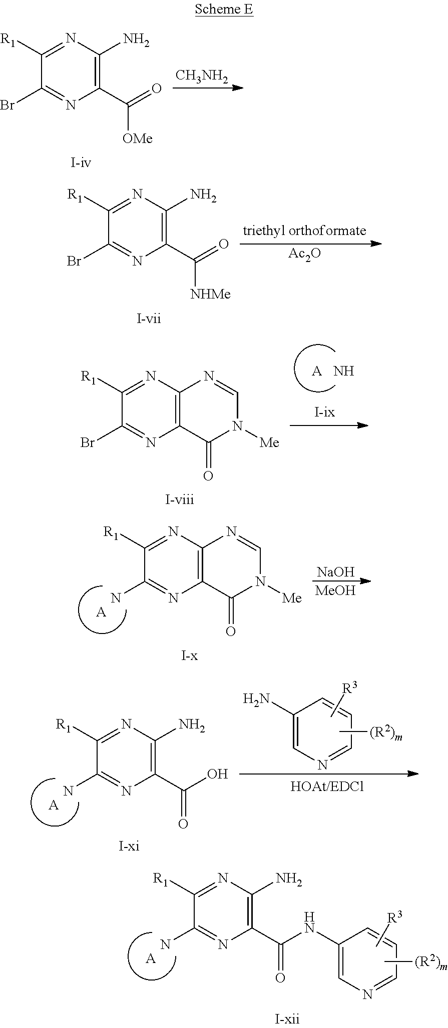

Alternatively, a bromopyrazine (I-iv) can be condensed with methyl amine under aqueous conditions to generate a secondary amide intermediate (I-vii). The secondary amide can then be treated with triethyl orthoformate under refluxing conditions to produce a 6-bromo-3-methylpteridin-4(3H)-one (I-viii). An intermediate 6-bromo-3-methylpteridin-4(3H)-one (I-viii) can then be reacted with a secondary amine at elevated temperature to produce intermediate I-x. The 3-methylpteridin-4(3H)-one (I-x) may be hydrolyzed to an aminopyrazine-2-carboxylic acid intermediate (I-xi), which can be further elaborated via a coupling to substituted amino pyridines in a polar solvent (e.g. DMF) containing to form intermediates I-xii.

### Example I: Preparation of Key Intermediates

### Example I.A: Preparation of 3-Amino-6-bromo-N-(4-morpholinopyridin-3-yl)pyrazine-2-carboxamide (Intermediate A)

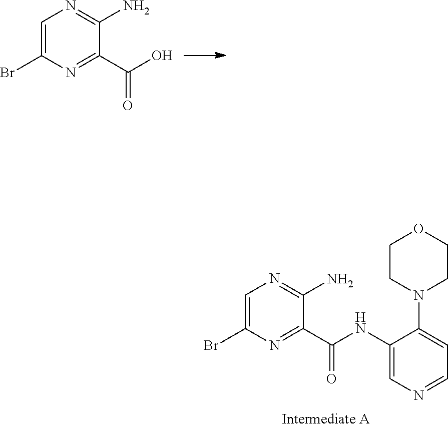 [SMILES: Nc1ncc(Br)nc1C(=O)O ; Nc1ncc(Br)nc1C(=O)Nc1cnccc1N1CCOCC1]

To a solution containing 1.63 g (7.5 mmol) of 2-amino-5-bromopyrazine carboxylic acid in 25 mL of DMF was added 3.54 g (9.36 mmol) of HATU. The reaction mixture was allowed to stir for 15 minutes before the addition of 1.68 g (9.36 mmol) of 3-amino-4-morpholinopyridine and 2.5 mL (22.5 mmol) of N-methylmorpholine. The reaction was stirred for 16 h then quenched with a 10 mL of a saturated NaHCO₃ solution and extracted with EtOAc three times. The combined organic extracts were washed with brine and dried over Na₂SO₄. Evaporation of the solvent and column chromatography (SiO₂; 10% CH₃OH/DCM) provided Intermediate A as a yellow solid. ¹H NMR (CD₃OD) δ 9.51 (s, 1H), 8.76 (s, 1H), 8.39 (d, J=5.4 Hz, 1H), 7.94 (d, J=8.1 Hz, 2H), 7.39-7.27 (m, 8H), 5.10 (s, 2H), 3.81 (t, J=7.5 Hz, 4H), 2.96 (t, J=7.5 Hz, 4H). [M+H]⁺=568.1.

### Example I.B: Preparation of Benzyl (3-(4-(5-amino-6-(pyrazin-2-yl)phenyl)ethyl)carbamate (Intermediate B)

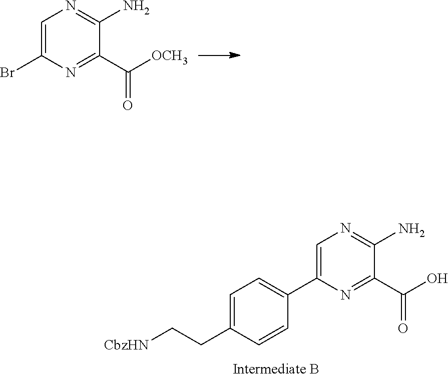 [SMILES: COC(=O)c1nc(Br)cnc1N ; Nc1ncc(-c2ccc(CCNC(=O)OCc3ccccc3)cc2)nc1C(=O)O]

To a solution of methyl 3-amino-6-bromopyrazine-2-carboxylate (4.6 g, 20.0 mmol) and 4-(2-(benzyloxycarbonylamino)ethyl)phenylboronic acid (6.58 g, 22.0 mmol) in 50 mL of dioxane was added 2M Na₂CO₃ solution (20 ml, 40.0 mmol). The reaction mixture was purged with a nitrogen before the addition of PdCl₂(dppf) (1.5 g, 2.0 mmol). The reaction mixture was heated at 90° C. for 1.5 h under a balloon of nitrogen. The reaction mixture was cooled and diluted with ethyl acetate and saturated NaHCO₃ solution. The organic layer was separated and dried over magnesium sulfate. The filtrate was concentrated and the residue was purified by silica gel chromatography (ISCO; 80 g cartridge: ethyl acetate/dichloromethane, 0-50%) to give the title compound as a light yellow solid which was dissolved in 60 mL of EtOH and 10 mL of THF. 30 mL (60 mmol) of 2N LiOH was added and the mixture was stirred at room temperature overnight. The reaction mixture was concentrated then treated with 2N HCl solution to pH=5 to effect a light yellow precipitate that was filtered and air dried for use without further purification. [M+H]⁺=392.2.

### Example LC: Preparation of Benzyl (4-(5-amino-6-((4-bromopyridin-3-yl)carbamoyl)pyrazin-2-yl)phenethyl)-carbamate (Intermediate C)

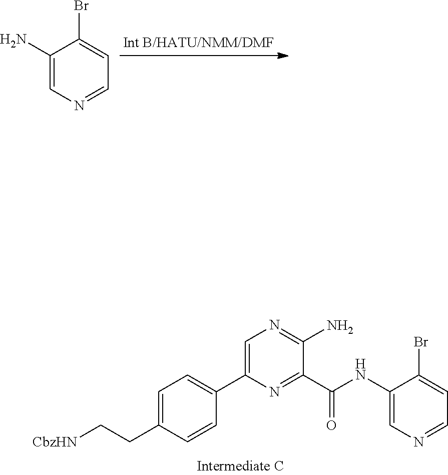 [SMILES: Nc1cnccc1Br ; Nc1ncc(-c2ccc(CCNC(=O)OCc3ccccc3)cc2)nc1C(=O)Nc1cnccc1Br]

To a solution containing 392 mg (1.0 mmol) of Intermediate B in 5 mL of DMF was added 456 mg (1.2 mmol) of HATU. The reaction mixture was allowed to stir for 15 minutes before the addition of 206 mg (1.2 mmol) of 3-amino-4-bromopyridine and 0.26 mL (2.4 mmol) of N-methylmorpholine. The reaction was stirred for 16 h then quenched with saturated NaHCO₃ solution and extracted with EtOAc. The combined organic extracts were washed with brine and dried over Na₂SO₄. Evaporation of the solvent and column chromatography (SiO₂; 10% CH₃OH/DCM) provided Intermediate C as a yellow solid. ¹H NMR (DMSO-d⁶) δ 10.6 (s, 1H), 9.25 (s, 1H), 9.01 (s, 1H), 8.29 (d, J=5.2 Hz, 1H), 8.10 (d, J=8.0 Hz, 2H), 7.8 (s, 1H), 7.65 (s, 2H), 7.57-7.51 (m, 8H), 5.02 (s, 2H), 3.32 (t, J=7.2 Hz, 2H), 2.79 (t, J=7.2 Hz, 2H), 1.23 (m, 1H). [M+H]⁺=547.4.

### Example I.D: Preparation of tert-Butyl 4-(3-methyl-4-oxo-3,4-dihydropteridin-6-yl)piperazine-1-carboxylate (Intermediate D)

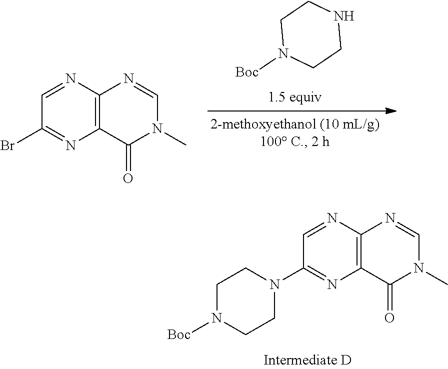 [SMILES: CC(C)(C)OC(=O)N1CCNCC1 ; Cn1cnc2ncc(Br)nc2c1=O ; Cn1cnc2ncc(N3CCN(C(=O)OC(C)(C)C)CC3)nc2c1=O]

To a solution of 6-bromo-3-methyl-pteridin-4-one (CAS #146940-38-7, 0.5 g, 2.1 mmol) in 2-methoxyethanol (10 mL/g) was added tert-butyl piperazine-1-carboxylate (0.584 g, 3.1 mmol, 1.5 equiv) and the resulting suspension heated at 100° C. for 2 hours. The reaction mixture was cooled in an ice bath and the collected precipitate was purified by preparative RP-HPLC (10→80% AcN (0.1% TFA) in H₂O (0.1% TFA)) to provide Intermediate D (0.35 g, 49% yield) as a yellow solid. [M+Na]⁺: 369.2, M-Boc: 246.2.

### Example I.E: Preparation of 3-Amino-6-(4-(tert-butoxycarbonyl)piperazin-1-yl)pyrazine-2-carboxylic acid (Intermediate E)

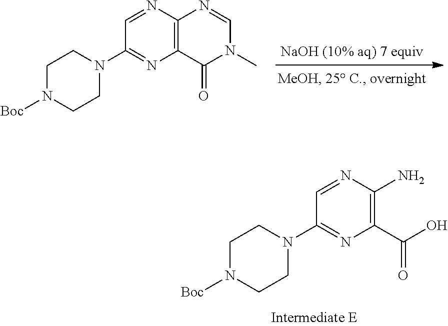 [SMILES: Cn1cnc2ncc(N3CCN(C(=O)OC(C)(C)C)CC3)nc2c1=O ; CC(C)(C)OC(=O)N1CCN(c2cnc(N)c(C(=O)O)n2)CC1]

To a solution of tert-butyl 4-(3-methyl-4-oxo-pteridin-6-yl)piperazine-1-carboxylate (0.165 g, 0.48 mmol) in MeOH (5 mL) was added NaOH (10% w/w, 7.0 equiv) and the resultant solution was stirred at ambient temperature for 17 hrs. The solution was then acidified, extracted with CH₂Cl₂, then treated with basic H₂O (pH ˜10) to precipitate the desired product which was redissolved in CH₂Cl₂, dried over Mg₂SO₄, filtered and concentrated to yield Intermediate E (0.10 g, 64% yield) as a yellow solid (90% purity). [M+Na]⁺: 346.2, M-Boc: 223.2.

### Examples 1-6: Preparation of Exemplary Amino-pyrazinecarboxamide Compounds

### Example 1.1: Preparation of Benzyl (4-(5-amino-6-((4-morpholinopyridin-3-yl)carbamoyl)pyrazin-2-yl)phenethyl)carbamate (Compound 1.1)

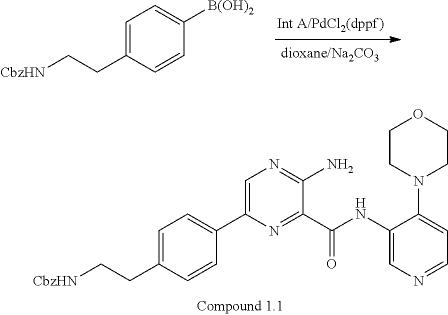 [SMILES: O=C(NCCc1ccc(B(O)O)cc1)OCc1ccccc1 ; Nc1ncc(-c2ccc(CCNC(=O)OCc3ccccc3)cc2)nc1C(=O)Nc1cnccc1N1CCOCC1]

To a solution of Intermediate A (567 mg, 1.0 mmol) and 4-(2-(benzyloxycarbonylamino)ethyl)phenylboronic acid (329 mg, 1.1 mmol) in 5 mL of dioxane was added 2M Na₂CO₃ solution (1.0 ml, 2.0 mmol). The reaction mixture was purged with a nitrogen before the addition of PdCl₂(dppf) (37 mg, 0.05 mmol). The reaction mixture was heated at 90° C. for 4 h under a balloon of nitrogen. The reaction mixture was cooled and diluted with ethyl acetate and saturated NaHCO₃ solution. The organic layer was separated and dried over magnesium sulfate. The filtrate was concentrated and the residue was purified by silica gel chromatography (ethyl acetate/dichloromethane, 0-50%) to Compound 1.1 as a light yellow solid.

### Example 2.1: Preparation of 3-Amino-6-(4-(3-aminopropyl)phenyl)-N-(4-morpholinopyridin-3-yl)pyrazine-2-carboxamide (Compound 2.1)

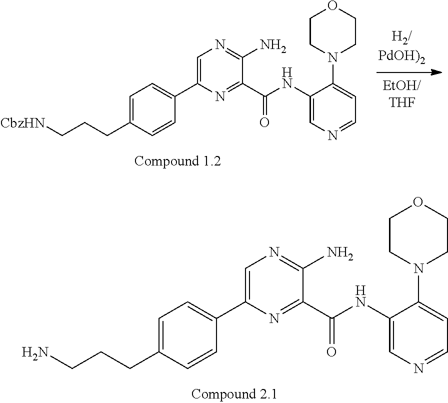 [SMILES: Nc1ncc(-c2ccc(CCCNC(=O)OCc3ccccc3)cc2)nc1C(=O)Nc1cnccc1N1CCOCC1 ; NCCCc1ccc(-c2cnc(N)c(C(=O)Nc3cnccc3N3CCOCC3)n2)cc1]

To a solution containing 758 mg (2.0 mmol) of the protected amine compound 1.2 in 10 mL of EtOH and 10 mL of THF was added 200 mg of 20% Pd(OH)₂ on carbon. The reaction mixture was degassed then capped with a balloon of H₂ then stirred at room temperature for 4 h. The reaction mixture was filtered through a pad of Celite and evaporated to afford the crude product which was purified by reverse phase HPLC to provide 750 mg of the TFA salt of Compound 2.1 as a yellow solid. ¹H NMR (CD₃OD) δ 9.49 (s, 1H), 8.71 (s, 1H), 8.27 (d, J=5.1 Hz, 1H), 7.94 (d, J=8.1 Hz, 2H), 7.35 (d, J=8.1 Hz, 2H), 7.24 (d, J=5.4 Hz, 1H), 3.80 (t, J=4.2 Hz, 4H), 3.00 (d, J=4.5 Hz, 4H), 2.71 (m, 4H), 1.82 (m, 2H). [M+H]⁺=434.1.

### Example 3.1: Preparation of Benzyl (4-(5-amino-6-((4-(2-methyl-1H-imidazol-1-yl)pyridin-3-yl)carbamoyl)pyrazin-2-yl)phenethyl)carbamate (Compound 3.1)

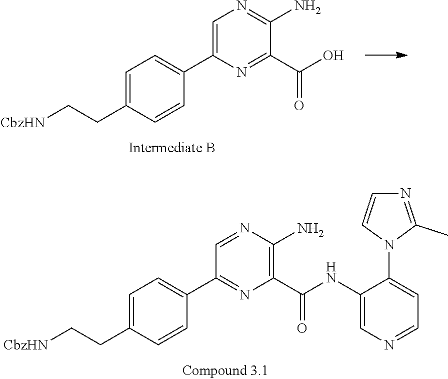 [SMILES: Nc1ncc(-c2ccc(CCNC(=O)OCc3ccccc3)cc2)nc1C(=O)O ; Cc1nccn1-c1ccncc1NC(=O)c1nc(-c2ccc(CCNC(=O)OCc3ccccc3)cc2)cnc1N]

To a solution containing 392 mg (1.0 mmol) of Intermediate B in 5 mL of DMF was added 456 mg (1.2 mmol) of HATU. The reaction mixture was allowed to stir for 15 minutes before the addition of 209 mg (1.2 mmol) of 4-(2-methyl-1H-imidazol-1-yl)pyridin-3-amine and 0.26 mL (2.4 mmol) of N-methylmorpholine. The reaction was stirred for 16 h then quenched with saturated NaHCO₃ solution and extracted with EtOAc. The combined organic extracts were washed with brine and dried over Na₂SO₄. Evaporation of the solvent and preparative HPLC provided Compound 3.1 as a yellow solid. ¹H NMR (CD₃OD) δ 9.81 (s, 1H), 8.74 (s, 1H), 8.53 (d, J=7.2 Hz, 1H), 7.64 (d, J=8.4 Hz, 2H), 7.54 (d, J=7.56 (s, 1H), 7.35-7.11 (m, 9H), 7.36-7.24 (m, 9H), 5.07 (s, 2H), 3.43 (t, J=7.5 Hz, 2H), 2.86 (t, J=7.5 Hz, 2H), 2.30 (s, 3H). [M+H]⁺=449.2.

### Example 4.1: Preparation of 3-Amino-6-(4-(2-aminoethyl)phenyl)-N-(4-(2-methyl-1H-imidazol-1-yl)pyridin-3-yl)pyrazine-2-carboxamide (Compound 4.1)

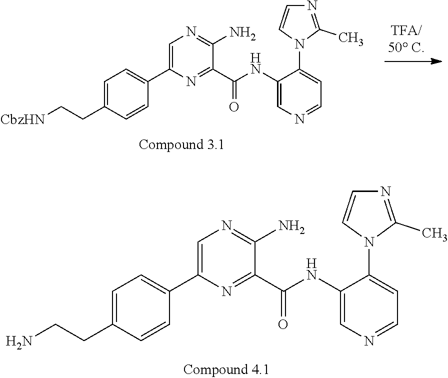 [SMILES: Cc1nccn1-c1ccncc1NC(=O)c1nc(-c2ccc(CCNC(=O)OCc3ccccc3)cc2)cnc1N ; Cc1nccn1-c1ccncc1NC(=O)c1nc(-c2ccc(CCN)cc2)cnc1N]

90 mg (0.20 mmol) of Compound 3.1 was dissolved in 2 mL of TFA and the mixture was heated at 50° C. for 4 h. The reaction mixture was evaporated to afford the crude product which was purified by reverse phase HPLC to provide 50 mg of the TFA salt of Compound 4.1 as a yellow solid. ¹H NMR (CD₃OD) δ 9.83 (s, 1H), 8.75 (s, 1H), 8.54 (d, J=8.0 Hz, 1H), 7.63 (d, J=8.0 Hz, 2H), 7.44 (s, 1H), 7.40-7.29 (m, 3H), 7.217 (s, 1H), 3.06 (m, 2H), 2.90 (m, 2H), 2.35 (s, 3H). [M+H]⁺=415.2

### Example 5.1: Preparation of Benzyl (4-(6-([4,4′-bipyridin]-3-ylcarbamoyl)-5-aminopyrazin-2-yl)phenethyl)carbamate (Compound 5.1)

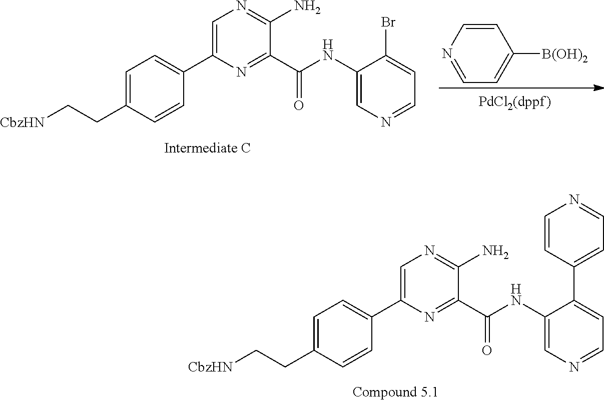

To a solution of Intermediate C (110 mg, 0.20 mmol) and 4-pyridineboronic acid (27 mg, 0.22 mmol) in 1 mL of dioxane was added 2M Na₂CO₃ solution (0.2 ml, 0.4 mmol). The reaction mixture was purged with a nitrogen before the addition of PdCl₂(dppf) (15 mg, 0.02 mmol). The reaction mixture was heated at 90° C. for 2 h under a balloon of nitrogen. The reaction mixture was cooled and diluted with ethyl acetate and saturated NaHCO₃ solution. The organic layer was separated and dried over magnesium sulfate. The filtrate was concentrated and the residue was purified by reverse phase column chromatography to give Compound 5.1 as a yellow solid. ¹H NMR (DMSO-d⁶) δ 10.2 (s, 1H), 9.32 (s, 1H), 8.85 (s, 1H), 8.68 (d, J=6.8 Hz, 2H), 8.5 (d, J=7.8 Hz, 1H), 7.66-7.22 (m, 15H), 4.99 (s, 2H), 3.28 (m, 2H), 2.74 (m, 2H).

### Example 6.1: Preparation of tert-Butyl 4-(5-amino-6-((4-morpholinopyridin-3-yl)carbamoyl)pyrazin-2-yl)piperazine-1-carboxylate (Compound 105)

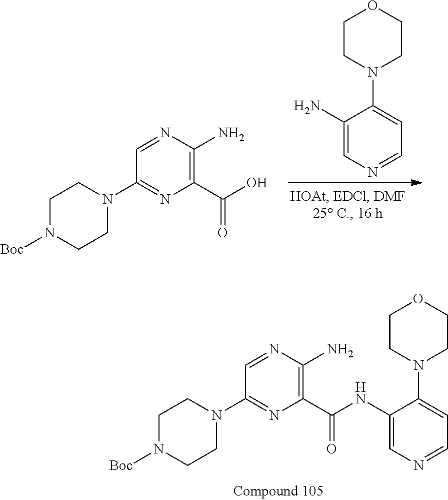 [SMILES: CC(C)(C)OC(=O)N1CCN(c2cnc(N)c(C(=O)O)n2)CC1 ; Nc1cnccc1N1CCOCC1 ; CC(C)(C)OC(=O)N1CCN(c2cnc(N)c(C(=O)Nc3cnccc3N3CCOCC3)n2)CC1]

To a solution of 3-Amino-6-(4-(tert-butoxycarbonyl)piperazin-1-yl)pyrazine-2-carboxylic acid (0.2 g, 0.62 mmol) in DMF (5.0 mL) was added HOAt (0.126 g, 0.93 mmol, 1.5 equiv) and EDCI (0.177 g, 0.93 mmol, 1.5 equiv) after the solution was allowed to stir at ambient temperature for 15 minutes, 3-amino-4-bromopyridine (0.166 g, 0.93 mmol, 1.5 equiv) was added as a solid and the resultant solution was left to stir at room temperature for 16 hours. The reaction was purified directly by preparative RP-HPLC (10→80% AcN (0.1% TFA) in H₂O (0.1% TFA)) to provide Compound 105 (0.080 g, 27% yield) as a yellow solid. [M+Na]: 406.2, M-Boc: 384.3.

### Example 7.1: Preparation of 3-Amino-N-(4-morpholinopyridin-3-yl)-6-(piperazin-1-yl)pyrazine-2-carboxamide (Compound 108)

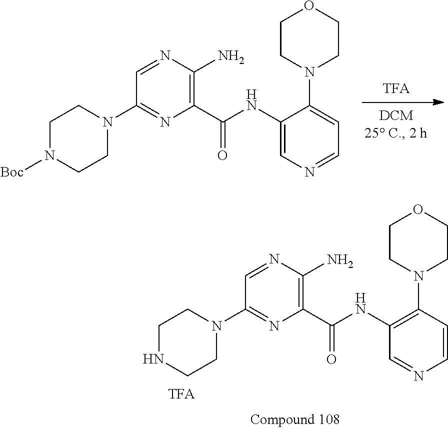 [SMILES: CC(C)(C)OC(=O)N1CCN(c2cnc(N)c(C(=O)Nc3cnccc3N3CCOCC3)n2)CC1 ; Nc1ncc(N2CCNCC2)nc1C(=O)Nc1cnccc1N1CCOCC1]

To a solution of tert-butyl 4-(5-amino-6-((4-morpholinopyridin-3-yl)carbamoyl)pyrazin-2-yl)piperazine-1-carboxylate (0.08 g, 0.165 mmol) in CH₂Cl₂ (1 mL) was added TFA (1 mL) at room temperature. The resultant solution was allowed to stir at ambient temperature for 2 hrs, it was then concentrated under reduced pressure and the residue was purified by preparative RP-HPLC (10→65% AcN (0.1% TFA) in H₂O (0.1% o TFA)) to provide Compound 108 (0.037 g, 45% yield) as a yellow solid. [M+H]⁺: 385.3.

The following compounds were prepared using the methodologies described herein in combination with the skill in the art:

TABLE 14

| Com- | | |
|---|---|---|
| pound | | |
| # | Structure | ¹H NMR/MS |
| 1.1 | 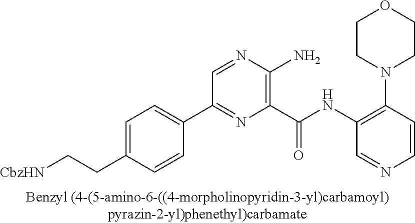 [SMILES: Nc1ncc(-c2ccc(CCNC(=O)OCc3ccccc3)cc2)nc1C(=O)Nc1cnccc1N1CCOCC1] | ¹H NMR (CD₃OD) δ 9.51 (s, 1H), 8.77 (s, 1H), 8.29 (d, J = 5.4 Hz, 1H), 7.99 (d, J = 8.1 Hz, 2H), 7.39-7.27 (m, 8H), 5.09 (s, 2H), 3.88 (bt, 2H), 3.22 (t, J = 9.2 Hz, 2H), 3.04 (bt, 2H), 1.92 (t, J = 9.6 Hz, 2H). |
| 1.2 | 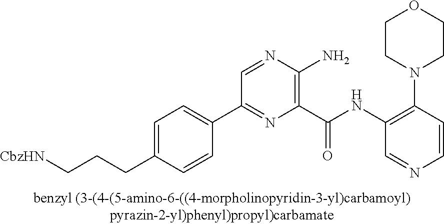 [SMILES: Nc1ncc(-c2ccc(CCCNC(=O)OCc3ccccc3)cc2)nc1C(=O)Nc1cnccc1N1CCOCC1] | ¹H NMR (300 MHz, Methanol-d₄) δ: 1.86- 1.94 (m, 2H), 2.74 (t, J = 7.2 Hz, 2H), 3.03 (t, J = 4.2 Hz, 4H), 3.20 (t, J = 6.9 Hz, 2H), 3.81-3.83 (m, 4H), 5.10 (s, 2H), 7.27-7.40 (m, 8H), 7.99 (d, J = 8.1 Hz, 2H), 8.29 (d, J = 5.4 Hz, 1H), 8.77 (s, 1H), 9.51 (s, 1H). |
| 2.1 | 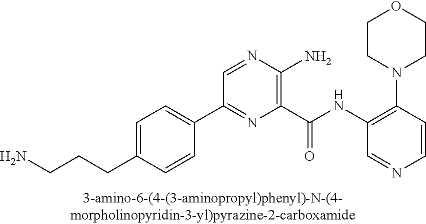 [SMILES: NCCCc1ccc(-c2cnc(N)c(C(=O)Nc3cnccc3N3CCOCC3)n2)cc1] | ¹H NMR (300 MHz, Methanol-d₄) δ: 1.80- 1.90 (m, 2H), 2.69-2.77 (m, 4H), 2.98-3.01 (m, 4H), 3.78-3.81 (m, 4H), 7.25 (d, J = 5.4 Hz, 1H), 7.35 (d, J = 8.1 Hz, 2H), 7.94 (d, J = 8.1 Hz, 2H), 8.27 (d, J = 5.4 Hz, 1H), 8.71 (s, 1H), 9.49 (s, 1H). |
| 3.1 | 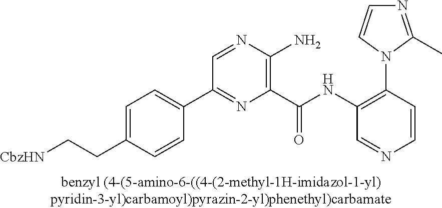 [SMILES: Cc1nccn1-c1ccncc1NC(=O)c1nc(-c2ccc(CCNC(=O)OCc3ccccc3)cc2)cnc1N] | [M + H]⁺: 549.3 |
| 4.1 | 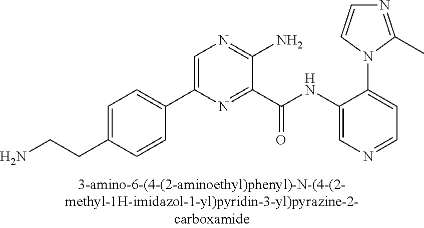 [SMILES: Cc1nccn1-c1ccncc1NC(=O)c1nc(-c2ccc(CCN)cc2)cnc1N] | ¹H NMR (400 MHz, Methanol-d₄) δ: 2.29 (s, 3H), 2.90-2.93 (m, 2H), 3.06-3.08 (m, 2H), 7.21 (s, 1H), 7.31-7.39 (m, 3H), 7.66 (s, 1H), 7.67 (d, J = 8.4 Hz, 2H), 8.53 (d, J = 5.2 Hz, 1H), 8.76 (s, 1H), 9.83 (s, 1H). |
| 5.1 | 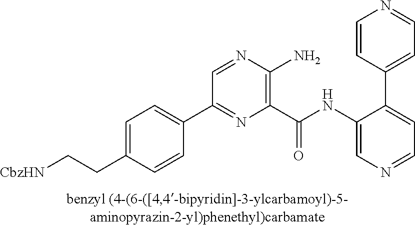 [SMILES: Nc1ncc(-c2ccc(CCNC(=O)OCc3ccccc3)cc2)nc1C(=O)Nc1cnccc1-c1ccncc1] | ¹H NMR (400 MHz, DMSO-d₄) δ: 2.61- 2.75 (m, 2H), 3.14-3.28 (m, 2H), 4.99 (s, 2H), 7.04-7.67 (m, 15H), 8.51 (m, 1H), 8.69-8.70 (m, 2H), 8.85 (s, 1H), 9.32 (s, 1H), 10.23 (s, 1H). |
| 6 | 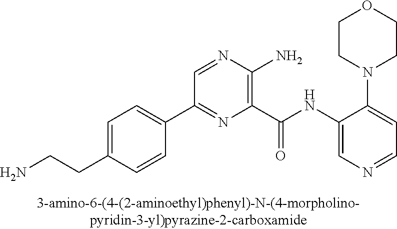 [SMILES: NCCc1ccc(-c2cnc(N)c(C(=O)Nc3cnccc3N3CCOCC3)n2)cc1] | ¹H NMR (300 MHz, DMSO-d₆ + D₂O) δ: 2.95-2.98 (m, 2H), 3.08-3.11 (m, 2H), 3.57 (s, 4H), 3.71 (s, 4H), 7.42 (d, J = 7.2 Hz, 3H), 8.14-8.16 (m, 2H), 8.33 (s, 1H), 8.73 (s, 1H), 8.94 (s, 1H). |
| 7 | 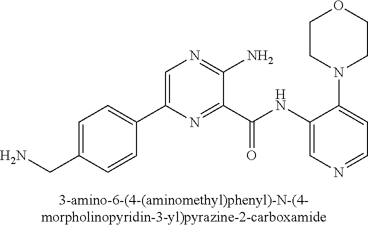 [SMILES: NCc1ccc(-c2cnc(N)c(C(=O)Nc3cnccc3N3CCOCC3)n2)cc1] | ¹H NMR (300 MHz, DMSO-d₆) δ: 3.47 (s, 4H), 3.70 (s, 4H), 4.11-4.13 (m, 2H), 7.39 (d, J = 6.0 Hz, 1H), 7.58 (d, J = 8.1 Hz, 2H), 7.77 (s, 2H), 8.28-8.31 (m, 4H), 8.39 (d, J = 6.0 Hz, 1H), 8.84 (s, 1H), 9.04 (s, 1H), 10.48 (s, 1H). |
| 8 | 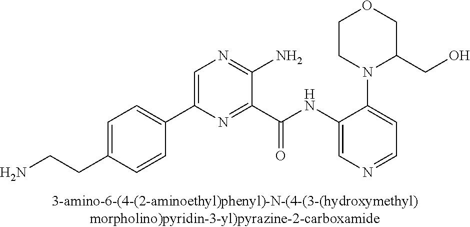 [SMILES: NCCc1ccc(-c2cnc(N)c(C(=O)Nc3cnccc3N3CCOCC3CO)n2)cc1] | [M + H]⁺: 450.3 |
| 9 | 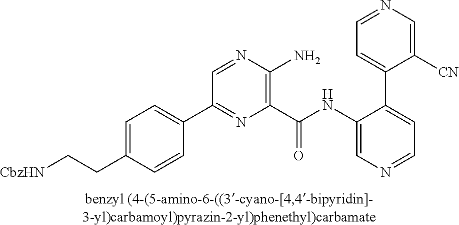 [SMILES: N#Cc1cnccc1-c1ccncc1NC(=O)c1nc(-c2ccc(CCNC(=O)OCc3ccccc3)cc2)cnc1N] | ¹H NMR (300 MHz, DMSO-d₆ + D₂O) δ: 2.75-2.82 (m, 2H), 3.27-3.31 (m, 2H), 5.01 (s, 2H), 7.30-7.38 (m, 7H), 8.21 (d, J = 7.8 Hz, 2H), 8.78 (d, J = 5.4 Hz, 1H), 8.89 (t, J = 5.7 Hz, 2H), 8.99 (s, 1H), 9.12 (d, J = 5.7 Hz, 1H), 9.42 (s, 1H), 9.63 (s, 1H). |
| 10 | 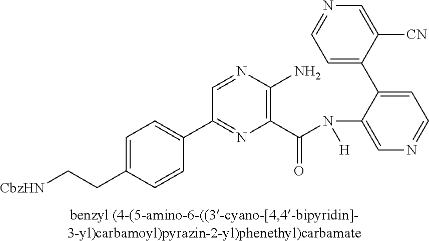 [SMILES: N#Cc1cnccc1-c1ccncc1NC(=O)c1nc(-c2ccc(CCNC(=O)OCc3ccccc3)cc2)cnc1N] | ¹H NMR (300 MHz, DMSO-d₆ + D₂O) δ: 2.80-2.84 (m, 2H), 3.28-3.33 (m, 2H), 5.02 (s, 2H), 7.33-7.39 (m, 7H), 8.20-8.25 (m, 2H), 8.23-9.20 (m, 5H), 9.63 (s, 1H), 9.75 (s, 1H). |
| 11 | 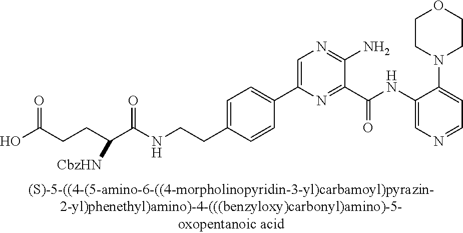 [SMILES: Nc1ncc(-c2ccc(CCNC(=O)[C@H](CCC(=O)O)NC(=O)OCc3ccccc3)cc2)nc1C(=O)Nc1cnccc1N1CCOCC1] | ¹H NMR (300 MHz, Methanol-d₄) δ: 1.80- 2.31 (m, 4H), 2.87-2.99 (m, 2H), 3.08 (s, 4H), 3.51-3.54 (m, 2H), 3.81 (s, 4H), 4.07 (s, 1H), 5.04 (s, 2H), 7.21-7.38 (m, 8H), 7.97-7.99 (m, 2H), 8.26-8.27 (m, 1H), 8.61- 8.75 (m, 1H), 9.46 (s, 1H). |
| 12 | 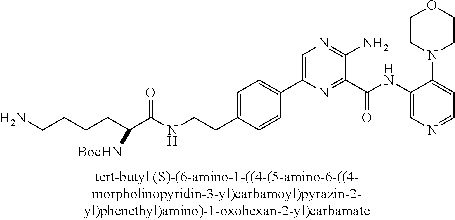 [SMILES: CC(C)(C)OC(=O)N[C@@H](CCCCN)C(=O)NCCc1ccc(-c2cnc(N)c(C(=O)Nc3cnccc3N3CCOCC3)n2)cc1] | ¹H NMR (400 MHz, Methanol-d₄) δ: 1.48- 1.54 (m, 15H), 2.61-2.64 (m, 2H), 2.98-2.93 (m, 2H), 3.03-3.06 (m, 4H), 3.47-3.55 (m, 2H), 3.83-3.85 (m, 5H), 7.28 (d, J = 5.2 Hz, 1H), 7.40-7.42 (m, 2H), 8.02 (d, J = 8.0 Hz, 2H), 8.29 (d, J = 5.6 Hz, 1H), 8.77 (s, 1H), 9.49 (s, 1H). |
| 13 | 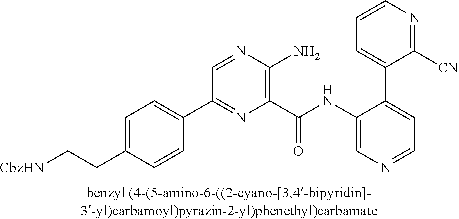 [SMILES: N#Cc1ncccc1-c1ccncc1NC(=O)c1nc(-c2ccc(CCNC(=O)OCc3ccccc3)cc2)cnc1N] | ¹H NMR (400 MHz, DMSO-d₆) δ: 2.84- 2.86 (m, 2H), 3.38 (s, 2H), 5.03 (s, 2H), 7.33-7.49 (m, 7H), 7.80-7.90 (m, 1H), 8.26- 8.28 (m, 3H), 8.71-8.75 (m, 2H), 9.10 (s, 1H), 9.29 (s, 1H), 9.36 (s, 1H), 9.48 (s, 1H), 13.19 (s, 1H). (two active hydrogens were not found) |
| 14 | 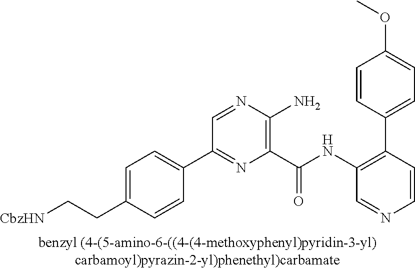 [SMILES: COc1ccc(-c2ccncc2NC(=O)c2nc(-c3ccc(CCNC(=O)OCc4ccccc4)cc3)cnc2N)cc1] | ¹H NMR (300 MHz, DMSO-d₆) δ: 2.78 (s, 2H), 3.28 (s, 2H), 3.79 (s, 3H), 5.03 (s, 2H), 7.12 (s, 2H), 7.21 (s, 2H), 7.35 (s, 7H), 7.58-7.91 (m, 6H), 8.44 (s, 1H), 8.90 (s, 1H), 9.54 (s, 1H), 10.25 (s, 1H). |
| 15 | 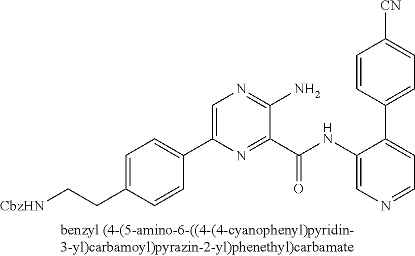 [SMILES: N#Cc1ccc(-c2ccncc2NC(=O)c2nc(-c3ccc(CCNC(=O)OCc4ccccc4)cc3)cnc2N)cc1] | [M + H]⁺: 570.3 |
| 16 | 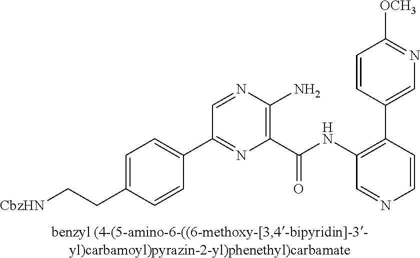 [SMILES: COc1ccc(-c2ccncc2NC(=O)c2nc(-c3ccc(CCNC(=O)OCc4ccccc4)cc3)cnc2N)cn1] | ¹H NMR (400 MHz, DMSO-d₆) δ: 2.77- 2.79 (m, 2H), 3.25-3.30 (m, 2H), 3.87 (s, 3H), 5.02 (s, 2H), 6.98 (d, J = 8.8 Hz, 1H), 7.22 (d, J = 8.0 Hz, 2H), 7.28-7.46 (m, 7H), 7.65-7.71 (m, 4H), 7.96-7.99 (m, 1H), 8.40 (s, 1H), 8.49 (d, J = 4.8 Hz, 1H), 8.90 (s, 1H), 9.35 (s, 1H), 10.28 (s, 1H). |
| 17 | 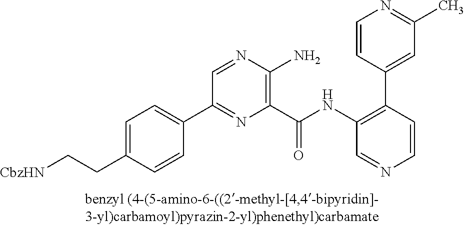 [SMILES: Cc1cc(-c2ccncc2NC(=O)c2nc(-c3ccc(CCNC(=O)OCc4ccccc4)cc3)cnc2N)ccn1] | ¹H NMR (300 MHz, DMSO-d₆) δ: 2.41 (s, 3H), 2.76-2.81 (m, 2H), 3.27-3.33 (m, 2H), 5.03 (s, 2H), 7.24-7.28 (m, 3H), 7.31-7.39 (m, 4H), 7.44-7.49 (m, 4H), 7.63 (d, J = 7.8 Hz, 4H), 8.52-8.61 (m, 2H), 8.89 (s, 1H), 9.42 (s, 1H), 10.24 (s, 1H). |
| 18 | 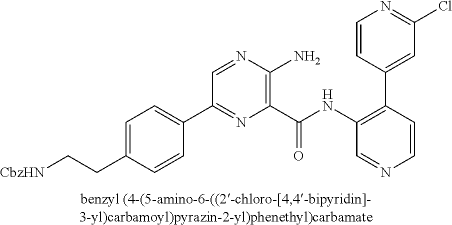 [SMILES: Nc1ncc(-c2ccc(CCNC(=O)OCc3ccccc3)cc2)nc1C(=O)Nc1cnccc1-c1ccnc(Cl)c1] | ¹H NMR (300 MHz, DMSO-d₆) δ: 2.72- 2.80 (m, 2H), 3.24-3.28 (m, 2H), 5.02 (s, 2H), 7.26-7.40 (m, 8H), 7.53-7.78 (m, 4H), 7.77-7.81 (m, 3H), 8.52-8.59 (m, 2H), 8.90 (s, 1H), 9.23 (s, 1H), 10.32 (s, 1H). |
| 19 | 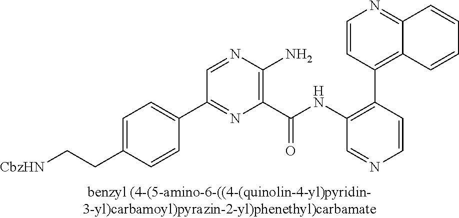 [SMILES: Nc1ncc(-c2ccc(CCNC(=O)OCc3ccccc3)cc2)nc1C(=O)Nc1cnccc1-c1ccnc2ccccc12] | ¹H NMR (400 MHz, DMSO-d₆) δ: 2.70- 2.79 (m, 2H), 3.29-3.31 (m, 2H), 5.06 (s, 2H), 7.06 (d, J = 8.0 Hz, 2H), 7.21-7.69 (m, 15H), 8.20-8.25 (m, 1H), 8.59 (d, J = 4.8 Hz, 1H), 8.77 (s, 1H), 9.11 (d, J = 4.4 Hz, 1H), 9.74 (d, J = 13.6 Hz, 2H). |
| 20 | 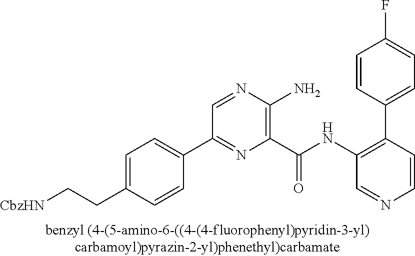 [SMILES: Nc1ncc(-c2ccc(CCNC(=O)OCc3ccccc3)cc2)nc1C(=O)Nc1cnccc1-c1ccc(F)cc1] | ¹H NMR (300 MHz, DMSO-d₆) δ: 2.77- 2.80 (m, 2H), 3.20-3.30 (m, 2H), 5.02 (s, 2H), 7.22-7.41 (m, 11H), 7.62-7.67 (m, 6H), 8.48 (d, J = 5.1 Hz, 1H), 8.89 (s, 1H), 9.45 (s, 1H), 10.19 (s, 1H). |
| 21 | 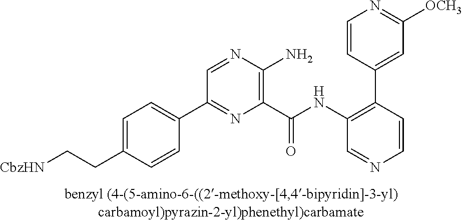 [SMILES: COc1cc(-c2ccncc2NC(=O)c2nc(-c3ccc(CCNC(=O)OCc4ccccc4)cc3)cnc2N)ccn1] | ¹H NMR (400 MHz, DMSO-d₆) δ: 2.77- 2.80 (m, 2H), 3.25-3.33 (m, 2H), 3.84 (s, 3H), 5.02 (s, 2H), 7.06 (s, 1H), 7.22 (d, J = 4.8 Hz, 1H), 7.23-7.37 (m, 7H), 7.39-7.40 (m, 1H), 7.47 (d, J = 4.8 Hz, 1H), 7.65-7.70 (m, 4H), 8.34 (d, J = 5.6 Hz, 1H), 8.53 (d, J = 4.8 Hz, 1H), 8.90 (s, 1H), 9.40 (s, 1H), 10.26 (s, 1H). |
| 22 | 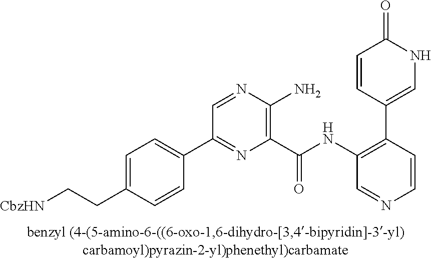 [SMILES: Nc1ncc(-c2ccc(CCNC(=O)OCc3ccccc3)cc2)nc1C(=O)Nc1cnccc1-c1ccc(=O)[nH]c1] | ¹H NMR (400 MHz, DMSO-d₆) δ: 2.77- 2.79 (m, 2H), 3.27-3.28 (m, 2H), 5.02 (s, 2H), 6.45 (d, J = 8.8 Hz, 1H), 7.30-7.47 (m, 9H), 7.68-7.73 (m, 4H), 7.87 (d, J = 7.6 Hz, 2H), 8.46-8.48 (m, 1H), 8.92 (s, 1H), 9.25 (s, 1H), 10.31 (s, 1H), 12.09 (s, 1H). |
| 23 | 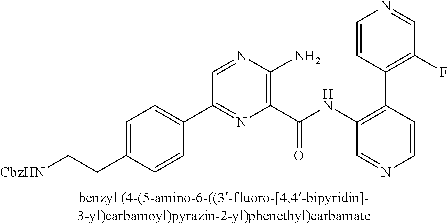 [SMILES: Nc1ncc(-c2ccc(CCNC(=O)OCc3ccccc3)cc2)nc1C(=O)Nc1cnccc1-c1ccncc1F] | ¹H NMR (300 MHz, DMSO-d₆) δ: 2.73- 2.81 (m, 2H), 3.24-3.34 (m, 2H), 5.03 (s, 2H), 7.26-7.43 (m, 8H), 7.54-7.71 (m, 6H), 8.57-8.60 (m, 2H), 8.77 (s, 1H), 8.90 (s, 1H), 9.33 (s, 1H), 10.19 (s, 1H). |
| 24 | 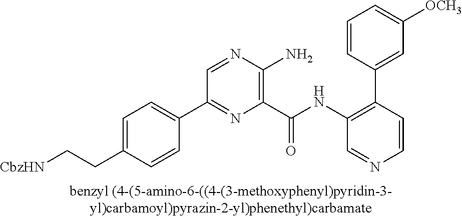 [SMILES: COc1cccc(-c2ccncc2NC(=O)c2nc(-c3ccc(CCNC(=O)OCc4ccccc4)cc3)cnc2N)c1] | ¹H NMR (300 MHz, DMSO-d₆) δ: 2.78- 2.82 (m, 2H), 3.27-3.34 (m, 2H), 3.68 (s, 3H), 5.03 (s, 2H), 7.13-7.63 (m, 17H), 8.47 (d, J = 5.1 Hz, 1H), 8.90 (s, 1H), 9.52 (s, 1H), 10.25 (s, 1H). |
| 25 | 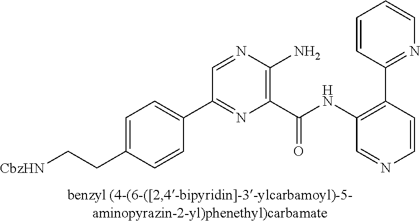 [SMILES: Nc1ncc(-c2ccc(CCNC(=O)OCc3ccccc3)cc2)nc1C(=O)Nc1cnccc1-c1ccccn1] | ¹H NMR (300 MHz, DMSO-d₆) δ: 2.82- 2.86 (m, 2H), 3.34-3.37 (m, 2H), 5.02 (s, 2H), 7.26-7.44 (m, 8H), 7.49-7.53 (m, 1H), 7.73 (s, 2H), 7.83 (d, J = 5.1 Hz, 1H), 7.99- 8.07 (m, 4H), 8.50 (d, J = 5.1 Hz, 1H), 8.59 (d, J = 4.8 Hz, 1H), 8.92 (s, 1H), 9.69 (s, 1H), 12.93 (s, 1H). |
| 26 | 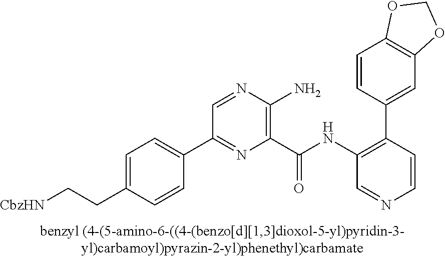 [SMILES: Nc1ncc(-c2ccc(CCNC(=O)OCc3ccccc3)cc2)nc1C(=O)Nc1cnccc1-c1ccc2c(c1)OCO2] | ¹H NMR (300 MHz, DMSO-d₆) δ: 2.76- 2.81 (m, 2H), 3.25-3.34 (m, 2H), 5.03 (s, 2H), 5.99 (s, 2H), 7.06-7.09 (m, 2H), 7.12 (s, 1H), 7.24-7.29 (m, 2H), 7.31-7.40 (m, 7H), 7.64-7.67 (m, 4H), 8.44 (d, J = 4.8 Hz, 1H), 8.91 (s, 1H), 9.51 (s, 1H), 10.27 (s, 1H). |
| 27 | 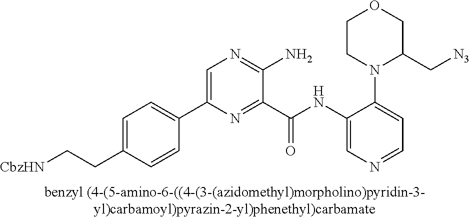 [SMILES: [N-]=[N+]=NCC1COCCN1c1ccncc1NC(=O)c1nc(-c2ccc(CCNC(=O)OCc3ccccc3)cc2)cnc1N] | [M + H]⁺: 609.3 |
| 28 | 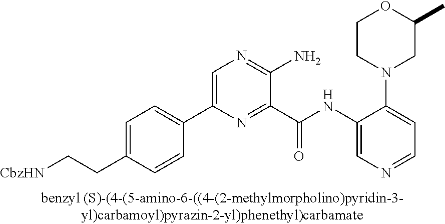 [SMILES: C[C@H]1CN(c2ccncc2NC(=O)c2nc(-c3ccc(CCNC(=O)OCc4ccccc4)cc3)cnc2N)CCO1] | [M + H]⁺: 568.3 |
| 29 | 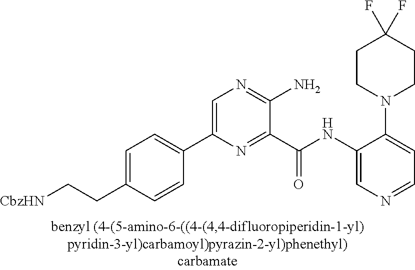 [SMILES: Nc1ncc(-c2ccc(CCNC(=O)OCc3ccccc3)cc2)nc1C(=O)Nc1cnccc1N1CCC(F)(F)CC1] | [M + H]⁺: 588.2 |
| 30 | 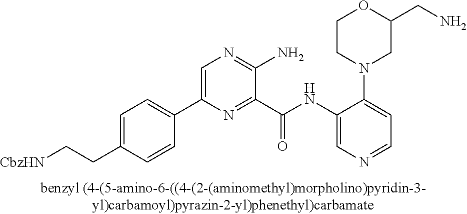 [SMILES: NCC1CN(c2ccncc2NC(=O)c2nc(-c3ccc(CCNC(=O)OCc4ccccc4)cc3)cnc2N)CCO1] | [M + H]⁺: 583.3 |
| 31 | 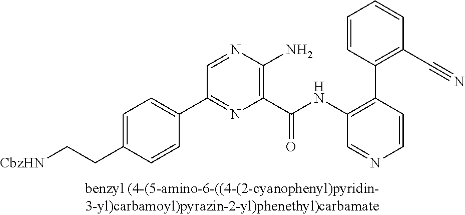 [SMILES: N#Cc1ccccc1-c1ccncc1NC(=O)c1nc(-c2ccc(CCNC(=O)OCc3ccccc3)cc2)cnc1N] | ¹H NMR (300 MHz, DMSO-d₆) δ: 1.23 (s, 2H), 2.69-2.81 (m, 2H), 4.98 (s, 2H), 7.57- 7.99 (m, 13H), 8.08-8.10 (m, 1H), 8.13-8.22 (m, 1H), 8.25-8.33 (m, 1H), 8.75 (d, J = 5.4 Hz, 1H), 8.81-8.83 (m, 1H), 9.00 (d, J = 9.0 Hz, 1H), 9.35 (s, 1H), 11.36 (s, 1H). |
| 32 | 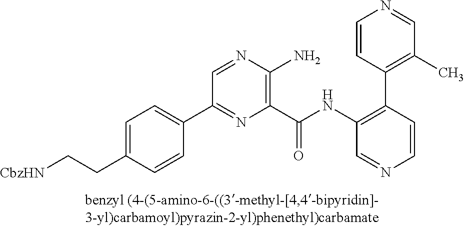 [SMILES: Cc1cnccc1-c1ccncc1NC(=O)c1nc(-c2ccc(CCNC(=O)OCc3ccccc3)cc2)cnc1N] | ¹H NMR (400 MHz, DMSO-d₆) δ: 2.09 (d, J = 9.2 Hz, 3H), 2.79 (s, 2H), 3.29 (d, J = 4.8 Hz, 2H), 5.04 (s, 2H), 7.28-7.44 (m, 12H), 7.66 (s, 2H), 8.52 (d, J = 4.4 Hz, 1H), 8.61 (s, 1H), 8.68 (s, 1H), 8.89 (s, 1H), 9.62 (s, 1H), 9.82 (s, 1H). |
| 33 | 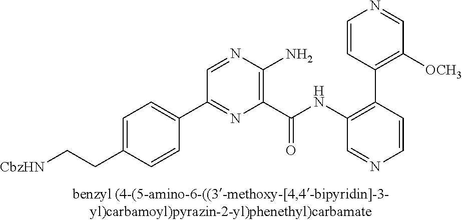 [SMILES: COc1cnccc1-c1ccncc1NC(=O)c1nc(-c2ccc(CCNC(=O)OCc3ccccc3)cc2)cnc1N] | ¹H NMR (400 MHz, DMSO-d₆) δ: 2.80 (t, J = 7.2 Hz, 2H), 3.27-3.31 (m, 2H), 3.76 (s, 3H), 5.03 (s, 2H), 7.27-7.46 (m, 10H), 7.54 (d, J = 8.0 Hz, 2H), 7.68 (s, 2H), 8.44 (d, J = 4.4 Hz, 1H), 8.49 (d, J = 4.8 Hz, 1H), 8.62 (s, 1H), 8.89 (s, 1H), 9.48 (s, 1H), 9.90 (s, 1H). |
| 34 | 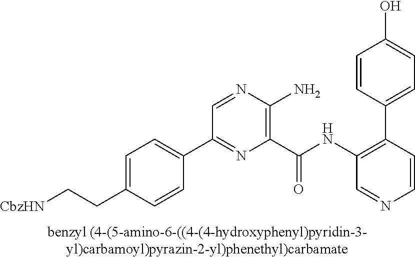 [SMILES: Nc1ncc(-c2ccc(CCNC(=O)OCc3ccccc3)cc2)nc1C(=O)Nc1cnccc1-c1ccc(O)cc1] | ¹H NMR (300 MHz, DMSO-d₆) δ: 2.78 (t, J = 8.4 Hz, 2H), 3.25-3.30 (m, 2H), 5.03 (s, 2H), 6.96 (d, J = 8.4 Hz, 2H), 7.27-7.36 (m, 8H), 7.40-7.43 (m, 3H), 7.63-7.70 (m, 4H), 8.41 (d, J = 4.8 Hz, 1H), 8.90 (s, 1H), 9.53 (s, 1H), 9.96 (s, 1H), 10.23 (s, 1H). |
| 35 | 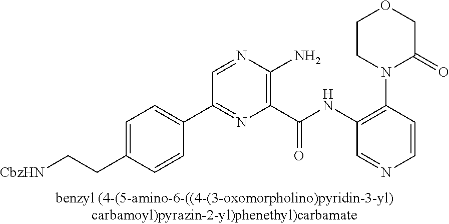 [SMILES: Nc1ncc(-c2ccc(CCNC(=O)OCc3ccccc3)cc2)nc1C(=O)Nc1cnccc1N1CCOCC1=O] | ¹H NMR (400 MHz, DMSO-d₆) δ: 2.77- 2.81 (m, 2H), 3.25-3.29 (m, 2H), 3.86 (s, 2H), 3.95-3.97 (m, 2H), 4.26 (s, 2H), 5.01 (s, 2H), 7.27-7.40 (m, 8H), 7.64-7.69 (m, 3H), 8.08-8.10 (m, 2H), 8.50 (s, 1H), 8.97 (s, 1H), 9.27 (s, 1H), 10.12 (s, 1H). |
| 36 | 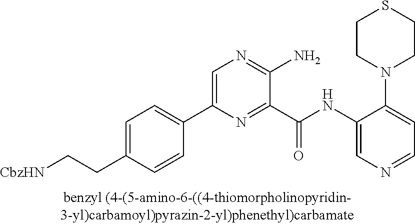 [SMILES: Nc1ncc(-c2ccc(CCNC(=O)OCc3ccccc3)cc2)nc1C(=O)Nc1cnccc1N1CCSCC1] | [M + H]⁺: 570.3 |
| 37 | 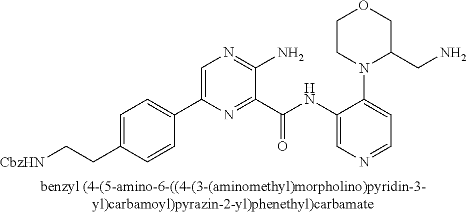 [SMILES: NCC1COCCN1c1ccncc1NC(=O)c1nc(-c2ccc(CCNC(=O)OCc3ccccc3)cc2)cnc1N] | [M + H]⁺: 583.2 (TFA Salt) |
| 38 | 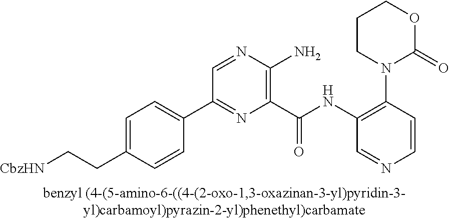 [SMILES: Nc1ncc(-c2ccc(CCNC(=O)OCc3ccccc3)cc2)nc1C(=O)Nc1cnccc1N1CCCOC1=O] | ¹H NMR (400 MHz, Acetonitrile-d₃) δ: 2.06-2.11 (m, 2H), 2.84-2.88 (m, 2H), 3.39- 3.44 (m, 2H), 3.84 (s, 2H), 4.39-4.41 (m, 2H), 5.05 (s, 2H), 5.72 (s, 1H), 7.28-7.38 (m, 7H), 7.44-7.45 (m, 1H), 8.00-8.02 (m, 2H), 8.46 (s, 1H), 8.81 (s, 1H), 9.36 (s, 1H), 10.22 (s, 1H). (two active hydrogens were not found) |
| 39 | 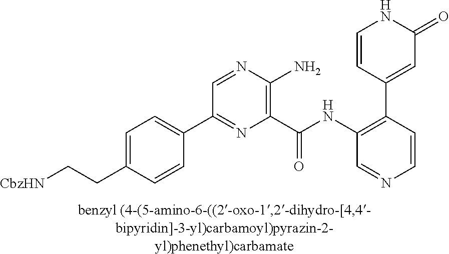 [SMILES: Nc1ncc(-c2ccc(CCNC(=O)OCc3ccccc3)cc2)nc1C(=O)Nc1cnccc1-c1cc[nH]c(=O)c1] | ¹H NMR (400 MHz, DMSO-d₆) δ: 2.78- 2.81 (m, 2H), 3.27-3.36 (m, 2H), 5.02 (s, 2H), 6.34 (d, J = 6.4 Hz, 1H), 6.52 (s, 1H), 7.29-7.37 (m, 7H), 7.43 (d, J = 4.8 Hz, 2H), 7.52 (d, J = 6.8 Hz, 1H), 7.67 (s, 2H), 7.79 (d, J = 8.4 Hz, 2H), 8.48 (d, J = 4.8 Hz, 1H), 8.92 (s, 1H), 9.42 (s, 1H), 10.24 (s, 1H), 11.97 (s, 1H). |
| 40 | 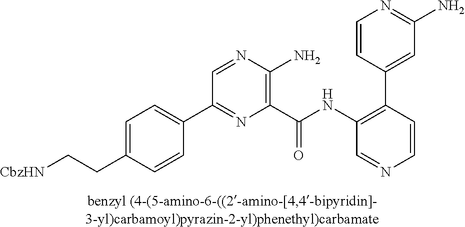 [SMILES: Nc1cc(-c2ccncc2NC(=O)c2nc(-c3ccc(CCNC(=O)OCc4ccccc4)cc3)cnc2N)ccn1] | ¹H NMR (300 MHz, DMSO-d₆) δ: 2.77- 2.82 (m, 2H), 3.28-3.30 (m, 2H), 5.03 (s, 2H), 6.24 (s, 2H), 6.53 (s, 1H), 6.67 (d, J = 3.6 Hz, 1H), 1.30-7.44 (m, 9H), 7.68 (d, J = 7.8 Hz, 4H), 8.08 (d, J = 5.4 Hz, 1H), 8.48 (d, J = 4.8 Hz, 1H), 8.92 (s, 1H), 9.57 (s, 1H), 10.19 (s, 1H). |
| 41 | 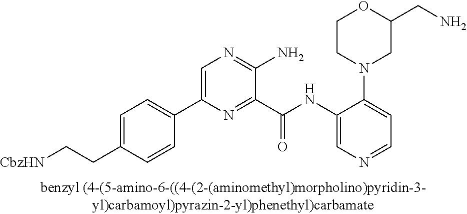 [SMILES: NCC1CN(c2ccncc2NC(=O)c2nc(-c3ccc(CCNC(=O)OCc4ccccc4)cc3)cnc2N)CCO1] | [M + H]⁺: 583.3 |
| 42 | 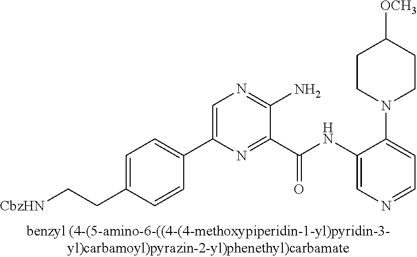 [SMILES: COC1CCN(c2ccncc2NC(=O)c2nc(-c3ccc(CCNC(=O)OCc4ccccc4)cc3)cnc2N)CC1] | [M + H]⁺: 582.3 |
| 43 | 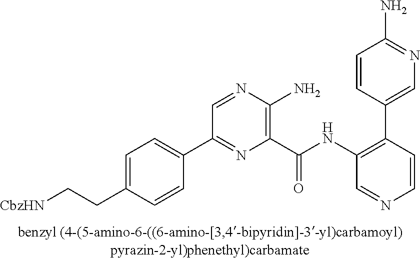 [SMILES: Nc1ccc(-c2ccncc2NC(=O)c2nc(-c3ccc(CCNC(=O)OCc4ccccc4)cc3)cnc2N)cn1] | ¹H NMR (300 MHz, DMSO-d₆) δ: 2.74- 2.82 (m, 2H), 3.28-3.32 (m, 2H), 5.03 (s, 2H), 6.40 (s, 2H), 6.55-6.58 (m, 1H), 7.30- 7.43 (m, 9H), 7.64-7.67 (m, 3H), 7.79-7.82 (m, 2H), 8.16 (s, 1H), 8.44 (d, J = 4.8 Hz, 1H), 8.92 (s, 1H), 9.33 (s, 1H), 10.30 (s, 1H). |
| 44 | 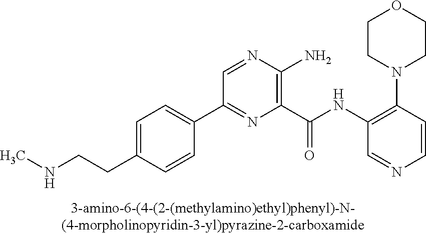 [SMILES: CNCCc1ccc(-c2cnc(N)c(C(=O)Nc3cnccc3N3CCOCC3)n2)cc1] | ¹H NMR (400 MHz, Acetonitrile-d₃) δ: 2.39 (s, 4H), 2.85 (s, 4H), 2.96-2.99 (m, 4H), 3.75-3.77 (m, 4H), 7.17 (d, J = 5.2 Hz, 1H), 7.41 (d, J = 8.0 Hz, 2H), 8.04 (d, J = 8.4 Hz, 2H), 8.33 (d, J = 5.2 Hz, 1H), 8.80 (s, 1H), 9.44 (s, 1H), 10.50 (s, 1H). (two active hydrogens were not found) |
| 45 | 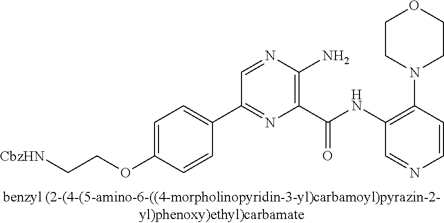 [SMILES: Nc1ncc(-c2ccc(OCCNC(=O)OCc3ccccc3)cc2)nc1C(=O)Nc1cnccc1N1CCOCC1] | ¹H NMR (400 MHz, DMSO-d₆) δ: 2.95- 2.96 (m, 4H), 3.40-3.44 (m, 2H), 3.71 (s, 4H), 4.03-4.07 (m, 2H), 5.05 (s, 2H), 7.02- 7.08 (m, 2H), 7.24 (d, J = 5.6 Hz, 1H), 7.28-7.34 (m, 5H), 7.51-7.54 (m, 1H), 7.56 (s, 2H), 8.09 (d, J = 8.4 Hz, 2H), 8.32 (d, J = 5.2 Hz, 1H), 8.92 (s, 1H), 9.34 (s, 1H), 9.35 (s, 1H). |
| 46 | 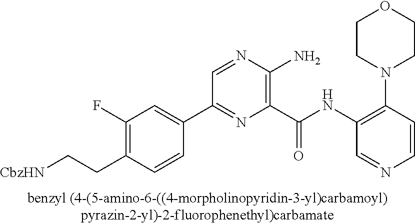 [SMILES: Nc1ncc(-c2ccc(CCNC(=O)OCc3ccccc3)c(F)c2)nc1C(=O)Nc1cnccc1N1CCOCC1] | ¹H NMR (300 MHz, DMSO-d₆) δ: 2.81- 2.86 (m, 2H), 2.99 (s, 4H), 3.26-3.28 (m, 2H), 3.73 (s, 4H), 5.02 (s, 2H), 7.25-7.57 (m, 8H), 7.83-8.01 (m, 4H), 8.34 (s, 1H), 8.99 (s, 1H), 9.28 (s, 1H), 10.38 (s, 1H). |
| 47 | 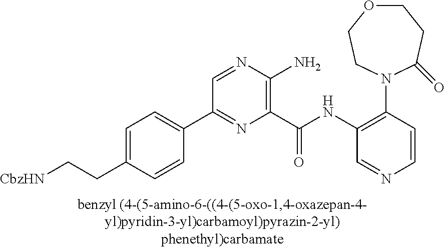 [SMILES: Nc1ncc(-c2ccc(CCNC(=O)OCc3ccccc3)cc2)nc1C(=O)Nc1cnccc1N1CCOCCC1=O] | ¹H NMR (400 MHz, DMSO-d₆) δ: 2.79- 2.94 (m, 5H), 3.56-3.95 (m, 7H), 5.01 (s, 2H), 7.20-7.48 (m, 9H), 7.70-8.07 (m, 4H), 8.47 (s, 1H), 8.97-9.10 (m, 2H), 10.05 (s, 1H). |
| 48 | 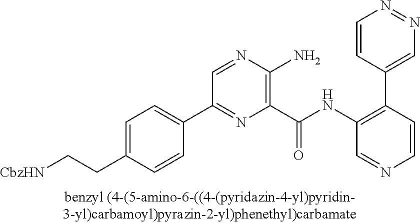 [SMILES: Nc1ncc(-c2ccc(CCNC(=O)OCc3ccccc3)cc2)nc1C(=O)Nc1cnccc1-c1ccnnc1] | ¹H NMR (400 MHz, Acetonitrile-d₃) δ: 2.84-2.87 (m, 2H), 3.39-3.44 (m, 2H), 5.07 (s, 2H), 5.82 (s, 1H), 7.28-7.37 (m, 7H), 7.39 (s, 1H), 7.45 (d, J = 4.8 Hz, 2H), 7.75- 7.77 (m, 1H), 8.58-8.71 (m, 1H), 8.74 (s, 1H), 9.26-9.41 (m, 3H), 9.98 (s, 1H). (two active hydrogens were not found) |
| 49 | 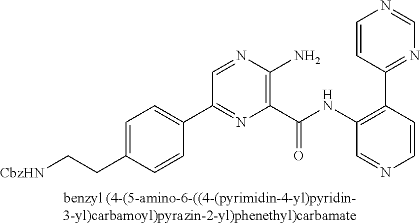 [SMILES: Nc1ncc(-c2ccc(CCNC(=O)OCc3ccccc3)cc2)nc1C(=O)Nc1cnccc1-c1ccncn1] | ¹H NMR (400 MHz, DMSO-d₆) δ: 2.74- 2.84 (m, 2H), 3.31 (d, J = 4.5 Hz, 2H), 5.03 (s, 2H), 7.21-7.55 (m, 8H), 7.73 (s, 2H), 7.84-8.03 (m, 3H), 8.19 (d, J = 4.5 Hz, 1H), 8.55 (s, 1H), 8.93 (s, 1H), 9.05 (d, J = 6.9 Hz, 1H), 9.15 (s, 1H), 9.73 (s, 1H), 12.72 (s, 1H). |
| 50 | 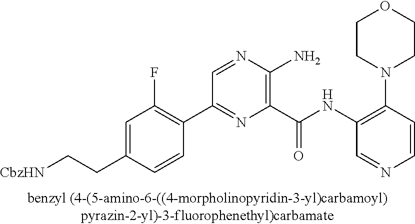 [SMILES: Nc1ncc(-c2ccc(CCNC(=O)OCc3ccccc3)cc2F)nc1C(=O)Nc1cnccc1N1CCOCC1] | ¹H NMR (400 MHz, DMSO-d₆) δ: 2.81- 2.84 (m, 2H), 2.93-2.95 (m, 4H), 3.27-3.31 (m, 2H), 3.63-3.64 (m, 4H), 5.01 (s, 2H), 7.21-7.39 (m, 10H), 7.83 (s, 1H), 7.95-7.99 (m, 1H), 8.31 (d, J = 5.6 Hz, 1H), 8.71 (s, 1H), 9.33 (s, 1H), 10.37 (s, 1H). |
| 51 | 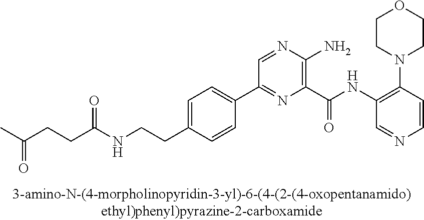 [SMILES: CC(=O)CCC(=O)NCCc1ccc(-c2cnc(N)c(C(=O)Nc3cnccc3N3CCOCC3)n2)cc1] | ¹H NMR (300 MHz, Methanol-d₄) δ: 1.39 (s, 1H), 2.17 (s, 3H), 2.41-2.43 (m, 2H), 2.87 (t, J = 7.2 Hz, 2H), 3.01-3.05 (m, 5H), 3.44-3.47 (m, 2H), 3.80-3.81 (m, 4H), 7.28 (d, J = 5.5 Hz, 1H), 7.38-7.42 (m, 2H), 7.86-8.10 (m, 2H), 8.28 (d, J = 5.2 Hz, 1H), 8.74-8.76 (m, 1H), 9.45 (s, 1H). |
| 52 | 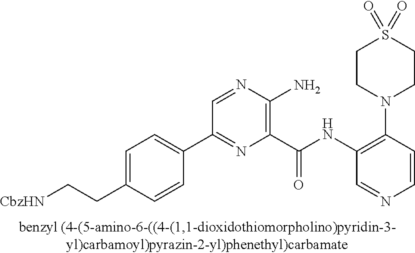 [SMILES: Nc1ncc(-c2ccc(CCNC(=O)OCc3ccccc3)cc2)nc1C(=O)Nc1cnccc1N1CCS(=O)(=O)CC1] | ¹H NMR (400 MHz, Acetonitrile-d₃) δ: 2.79-2.86 (m, 3H), 3.04 (s, 3H), 3.39-3.47 (m, 6H), 5.03-5.08 (m, 2H), 5.87 (s, 1H), 7.47-7.62 (m, 10H), 7.95-7.97 (m, 2H), 8.34-8.35 (m, 1H), 8.75-8.78 (m, 1H), 9.44 (s, 1H), 10.51 (s, 1H). |
| 53 | 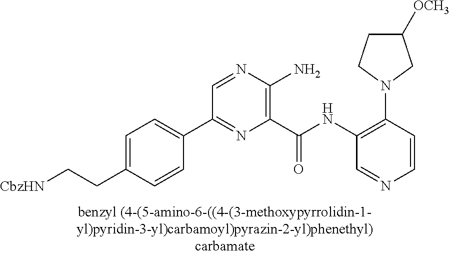 [SMILES: COC1CCN(c2ccncc2NC(=O)c2nc(-c3ccc(CCNC(=O)OCc4ccccc4)cc3)cnc2N)C1] | ¹H NMR (400 MHz, Methanol-d₄) δ: 1.97- 2.02 (m, 1H), 2.05-2.08 (m, 1H), 2.83-2.87 (m, 2H), 3.27-3.33 (m, 3H), 3.37-3.41 (m, 2H), 3.50-3.71 (m, 4H), 4.01 (s, 1H), 5.06 (s, 2H), 6.74 (d, J = 6.0 Hz, 1H), 7.22-7.34 (m, 7H), 8.00 (d, J = 8.0 Hz, 2H), 8.06 (d, J = 6.0 Hz, 1H), 8.19 (s, 1H), 8.76 (s, 1H). |
| 54 | 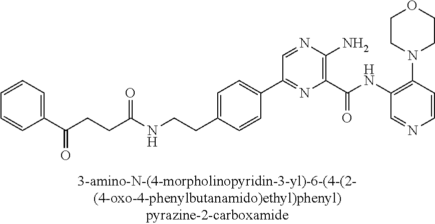 [SMILES: Nc1ncc(-c2ccc(CCNC(=O)CCC(=O)c3ccccc3)cc2)nc1C(=O)Nc1cnccc1N1CCOCC1] | ¹H NMR (400 MHz, DMSO-d₆) δ: 2.46- 2.50 (m, 2H), 2.77-2.89 (m, 2H), 2.96 (s, 4H), 3.21-3.31 (m, 4H), 3.73 (s, 4H), 7.23 (d, J = 5.2 Hz, 1H), 7.40-7.60 (m, 2H), 7.35 (d, J = 8 Hz, 2H), 7.62 (t, J = 7.2 Hz, 1H), 7.74 (s, 2H), 7.97 (d, J = 7.2 Hz, 2H), 8.03- 8.10 (m, 3H), 8.32 (d, J = 5.2 Hz, 1H), 8.95 (s, 1H), 9.33 (s, 1H), 10.43 (s, 1H). |
| 55 | 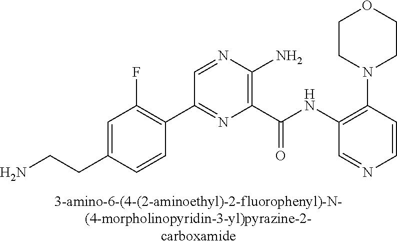 [SMILES: NCCc1ccc(-c2cnc(N)c(C(=O)Nc3cnccc3N3CCOCC3)n2)c(F)c1] | ¹H NMR (400 MHz, DMSO-d₆) δ: 2.93- 2.97 (m, 2H), 3.10-3.15 (m, 2H), 3.42 (s, 4H), 3.65-3.67 (m, 4H), 7.25-7.38 (m, 3H), 7.70 (s, 2H), 7.88 (s, 2H), 8.11-8.15 (m, 1H), 8.38 (d, J = 6.4 Hz, 1H), 8.73 (s, 1H), 8.88 (s, 1H), 10.40 (s, 1H). |
| 56 | 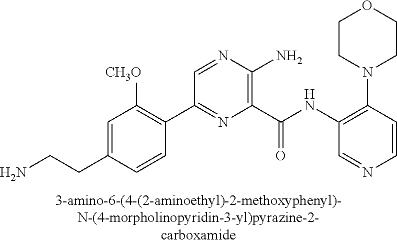 | ¹H NMR (300 MHz, Methanol-d₄) δ: 3.03- 3.09 (m, 2H), 3.24-3.32 (m, 2H), 3.46 (s, 4H), 3.75-3.87 (m, 4H), 3.95 (s, 3H), 7.06- 7.10 (m, 2H), 7.51 (d, J = 6.3 Hz, 1H), 7.86 (d, J = 7.5 Hz, 1H), 8.43 (s, 1H), 8.80 (s, 1H), 9.27 (s, 1H). |
| 57 | 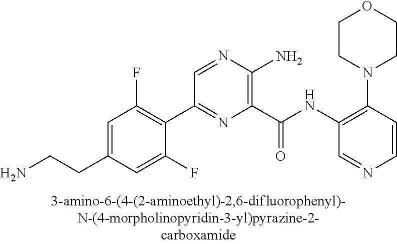 [SMILES: NCCc1cc(F)c(-c2cnc(N)c(C(=O)Nc3cnccc3N3CCOCC3)n2)c(F)c1] | ¹H NMR (300 MHz, DMSO-d₆) δ: 1.36 (s, 2H), 2.74-2.90 (m, 7H), 3.56 (s, 5H), 6.74 (s, 1H), 7.18 (t, J = 7.9 Hz, 3H), 7.88 (s, 2H), 8.29 (d, J = 5.4 Hz, 1H), 8.52 (s, 1H), 9.38 (s, 1H). |
| 58 | 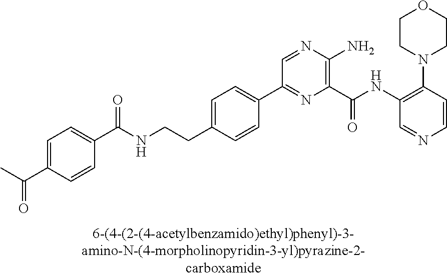 [SMILES: CC(=O)c1ccc(C(=O)NCCc2ccc(-c3cnc(N)c(C(=O)Nc4cnccc4N4CCOCC4)n3)cc2)cc1] | ¹H NMR (300 MHz, DMSO-d₆) δ: 2.62 (s, 3H), 2.95 (t, J = 7.3 Hz, 2H), 3.51-3.60 (m, 6H), 3.68-3.71 (m, 4H), 7.37-7.41 (m, 3H), 7.68 (s, 2H), 7.95 (d, J = 8.4 Hz, 2H), 8.03 (d, J = 8.7 Hz, 2H), 8.15 (d, J = 8.4 Hz, 2H), 8.38 (d, J = 6.6 Hz, 1H), 8.76-8.80 (m, 2H), 8.97 (s, 1H), 10.48 (s, 1H). |
| 59 | 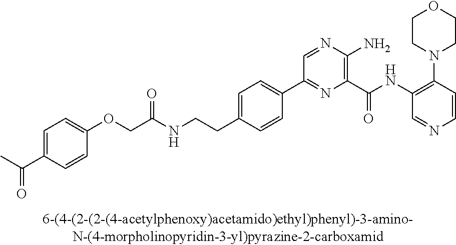 [SMILES: CC(=O)c1ccc(OCC(=O)NCCc2ccc(-c3cnc(N)c(C(=O)Nc4cnccc4N4CCOCC4)n3)cc2)cc1] | ¹H NMR (400 MHz, DMSO-d₆) δ: 2.44 (s, 3H), 2.85 (t, J = 7.2 Hz, 2H), 2.96 (t, J = 4.4 Hz, 4H), 3.40-3.42 (m, 2H), 3.72 (t, J = 4.4 Hz, 4H), 4.58 (s, 2H), 7.01 (d, J = 8.8 Hz, 2H), 7.24 (d, J = 5.2 Hz, 1H), 7.34 (d, J = 8.4 Hz, 2H), 7.73 (s, 2H), 7.89 (d, J = 8.8 Hz, 2H), 8.07 (d, J = 8.4 Hz, 2H), 8.24 (t, J = 5.6 Hz, 1H), 8.32 (d, J = 5.6 Hz, 1H), 8.95 (s, 1H), 9.34 (s, 1H), 10.44 (s, 1H). |
| 60 | 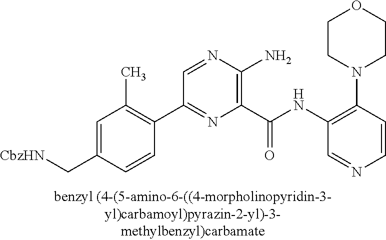 [SMILES: Cc1cc(CNC(=O)OCc2ccccc2)ccc1-c1cnc(N)c(C(=O)Nc2cnccc2N2CCOCC2)n1] | ¹H NMR (300 MHz, DMSO-d₆) δ: 2.41 (s, 3H), 2.89 (s, 4H), 3.28 (s, 4H), 4.23 (d, J = 6.0 Hz, 2H), 5.04 (s, 2H), 7.11-7.19 (m, 3H), 7.27-7.36 (m, 5H), 7.47 (d, J = 8.4 Hz, 1H), 7.67 (s, 2H), 7.74-7.85 (m, 1H), 8.27 (s, 1H), 8.49 (s, 1H), 9.31 (s, 1H), 10.31 (s, 1H). |
| 61 | 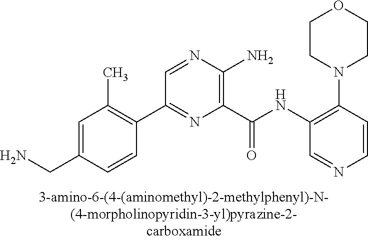 [SMILES: Cc1cc(CN)ccc1-c1cnc(N)c(C(=O)Nc2cnccc2N2CCOCC2)n1] | ¹H NMR (300 MHz, DMSO-d₆) δ: 2.40- 2.42 (m, 3H), 3.40 (s, 4H), 3.53-3.63 (m, 4H), 4.06-4.07 (m, 2H), 7.32-7.44 (m, 3H), 7.57-7.76 (m, 3H), 8.01-8.14 (m, 3H), 8.33 (s, 1H), 8.84 (s, 1H), 10.28 (s, 1H). |
| 62 | 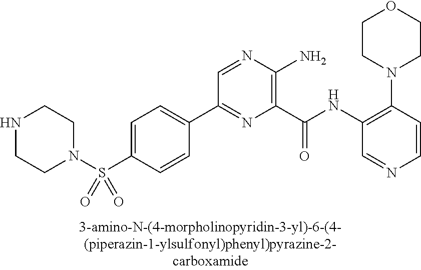 [SMILES: Nc1ncc(-c2ccc(S(=O)(=O)N3CCNCC3)cc2)nc1C(=O)Nc1cnccc1N1CCOCC1] | ¹H NMR (300 MHz, DMSO-d₆) δ: 3.18- 3.24 (m, 8H), 3.64-3.72 (m, 8H), 7.45 (d, J = 6.9 Hz, 1H), 7.88-7.92 (m, 3H), 8.42 (d, J = 6.9 Hz, 1H), 8.55 (d, J = 8.4 Hz, 2H), 8.68 (s, 1H), 8.89 (s, 2H), 9.12 (s, 1H), 10.63 (s, 1H). |
| 63 | 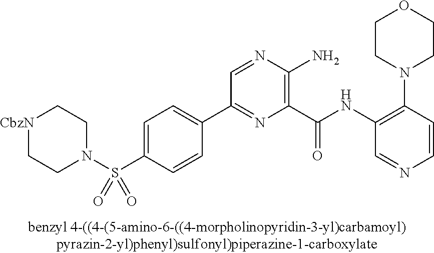 [SMILES: Nc1ncc(-c2ccc(S(=O)(=O)N3CCN(C(=O)OCc4ccccc4)CC3)cc2)nc1C(=O)Nc1cnccc1N1CCOCC1] | ¹H NMR (300 MHz, DMSO-d₆) δ: 2.93- 2.94 (m, 8H), 3.49 (s, 4H), 3.63 (s, 4H), 5.00 (s, 2H), 7.21-7.31 (m, 6H), 7.83-7.94 (m, 4H), 8.31-8.38 (m, 3H), 9.04 (s, 1H), 9.29 (s, 1H), 10.45 (s, 1H). |
| 64 | 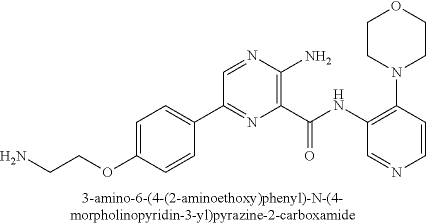 [SMILES: NCCOc1ccc(-c2cnc(N)c(C(=O)Nc3cnccc3N3CCOCC3)n2)cc1] | ¹H NMR (300 MHz, Methanol-d₄) δ: 3.01- 3.23 (m, 6H), 3.81-3.83 (m, 4H), 4.08-4.10 (m, 2H), 7.10 (d, J = 8.4 Hz, 2H), 7.26-7.34 (m, 1H), 7.98-8.01 (m, 2H), 8.27 (d, J = 5.4 Hz, 1H), 8.72 (s, 1H), 9.47 (s, 1H). |
| 65 | 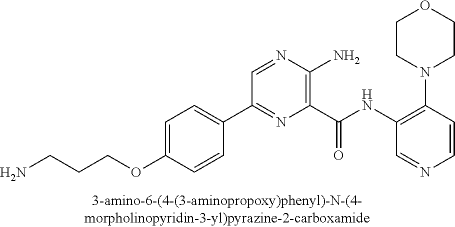 [SMILES: NCCCOc1ccc(-c2cnc(N)c(C(=O)Nc3cnccc3N3CCOCC3)n2)cc1] | ¹H NMR (300 MHz, DMSO-d₆) δ: 1.82- 1.86 (m, 2H), 2.72-2.77 (m, 2H), 2.96 (s, 4H), 3.72 (s, 4H), 4.10-4.16 (m, 2H), 7.04- 7.07 (m, 2H), 7.22-7.24 (m, 1H), 7.66-7.80 (m, 2H), 7.82-7.83 (m, 1H), 8.04-8.09 (m, 3H), 8.32 (d, J = 5.1 Hz, 1H), 8.91 (s, 1H), 9.33 (s, 1H), 10.50 (s, 1H). |
| 66 | 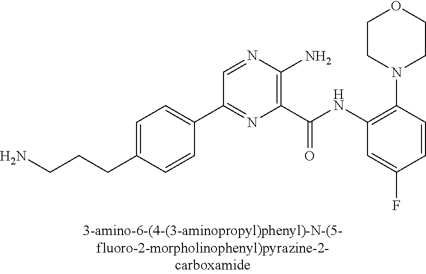 [SMILES: NCCCc1ccc(-c2cnc(N)c(C(=O)Nc3cc(F)ccc3N3CCOCC3)n2)cc1] | ¹H NMR (400 MHz, DMSO-d₆) δ: 1.85- 1.89 (m, 2H), 2.70-2.74 (m, 2H), 2.82-2.87 (m, 6H), 3.68-3.77 (m, 4H), 6.96-6.99 (m, 1H), 7.33-7.47 (m, 3H), 7.82 (s, 5H), 8.12 (d, J = 8.4 Hz, 1H), 8.28-8.31 (m, 1H), 8.95 (s, 1H), 11.06 (s, 1H). |
| 67 | 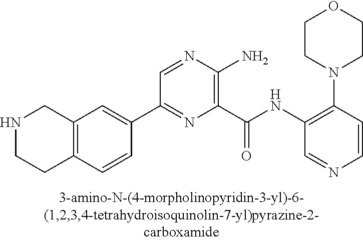 [SMILES: Nc1ncc(-c2ccc3c(c2)CNCC3)nc1C(=O)Nc1cnccc1N1CCOCC1] | ¹H NMR (300 MHz, Methanol-d₄) δ: 2.90- 2.92 (m, 2H), 2.94-3.05 (m, 4H), 3.11-3.18 (m, 2H), 3.76-3.84 (m, 4H), 4.85 (s, 2H), 7.24-7.29 (m, 2H), 7.78 (s, 1H), 7.85-7.87 (m, 1H), 8.27 (d, J = 5.4 Hz, 1H), 8.73 (s, 1H), 9.45 (s, 1H). |
| 68 | 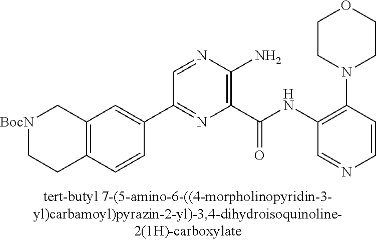 [SMILES: CC(C)(C)OC(=O)N1CCc2ccc(-c3cnc(N)c(C(=O)Nc4cnccc4N4CCOCC4)n3)cc2C1] | ¹H NMR (400 MHz, Methanol-d₄) δ: 1.53 (s, 9H), 2.92 (m, 2H), 3.02-3.32 (m, 4H), 3.69-3.71 (m, 2H), 3.80-3.82 (m, 4H), 4.85 (s, 2H), 7.24 (d, J = 5.6 Hz, 1H), 7.30 (d, J = 8.0 Hz, 1H), 7.78 (s, 1H), 7.86 (d, J = 8.0 Hz, 1H), 8.27 (d, J = 5.6 Hz, 1H), 8.73 (s, 1H), 9.45 (s, 1H). |
| 69 | 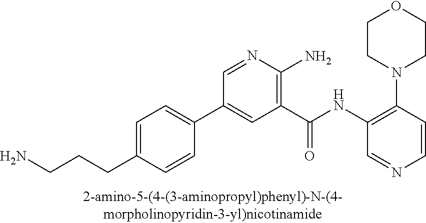 [SMILES: NCCCc1ccc(-c2cnc(N)c(C(=O)Nc3cnccc3N3CCOCC3)c2)cc1] | ¹H NMR (400 MHz, Methanol-d₄) δ: 1.98- 2.06 (m, 2H), 2.71-2.82 (m, 2H), 2.97-3.01 (m, 2H), 3.72-3.81 (m, 8H), 7.40-7.42 (m, 3H), 7.68-7.73 (m, 2H), 8.25-8.28 (m, 1H), 8.45-8.49 (m, 2H), 8.80 (s, 1H). |
| 70 | 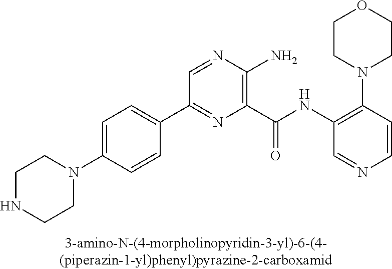 [SMILES: Nc1ncc(-c2ccc(N3CCNCC3)cc2)nc1C(=O)Nc1cnccc1N1CCOCC1] | ¹H NMR (400 MHz, Methanol-d₄) δ: 3.41- 3.50 (m, 4H), 3.52-3.61 (m, 8H), 3.82-3.84 (m, 4H), 7.19 (d, J = 8.8 Hz, 2H), 7.52 (d, J = 6.8 Hz, 1H), 8.02 (d, J = 8.8 Hz, 2H), 8.35-8.37 (m, 1H), 8.80 (s, 1H), 9.11 (d, J = 1.2 Hz, 1H). |
| 71 | 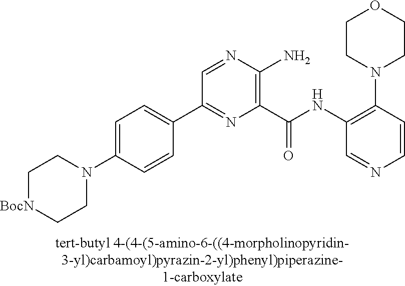 [SMILES: CC(C)(C)OC(=O)N1CCN(c2ccc(-c3cnc(N)c(C(=O)Nc4cnccc4N4CCOCC4)n3)cc2)CC1] | ¹H NMR (400 MHz, DMSO-d₆) δ: 1.43 (s, 9H), 3.05 (s, 4H), 3.20-3.22 (m, 4H), 3.57 (s, 4H), 3.72-3.73 (m, 4H), 7.07 (d, J = 8.8 Hz, 2H), 7.26 (d, J = 5.6 Hz, 1H), 7.60 (s, 2H), 8.03 (d, J = 8.8 Hz, 2H), 8.32 (d, J = 5.6 Hz, 1H), 8.90 (s, 1H), 9.25 (s, 1H), 10.44 (s, 1H). |
| 72 | 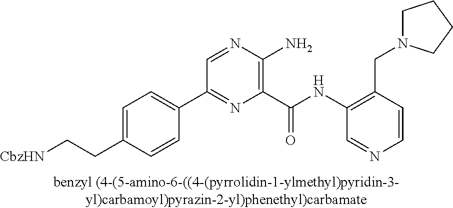 [SMILES: Nc1ncc(-c2ccc(CCNC(=O)OCc3ccccc3)cc2)nc1C(=O)Nc1cnccc1CN1CCCC1] | ¹H NMR (300 MHz, Methanol-d₄) δ: 1.55 (s, 4H), 2.45 (s, 4H), 2.84-2.89 (m, 2H), 3.37-3.42 (m, 2H), 3.77 (s, 2H), 5.08 (s, 2H), 7.28-7.35 (m, 8H), 7.82 (d, J = 7.8 Hz, 2H), 8.26 (d, J = 4.8 Hz, 1H), 8.64 (s, 1H), 9.52 (s, 1H). |
| 73 | 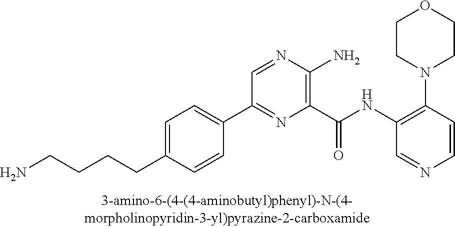 [SMILES: NCCCCc1ccc(-c2cnc(N)c(C(=O)Nc3cnccc3N3CCOCC3)n2)cc1] | ¹H NMR (300 MHz, Methanol-d₄) δ: 1.31- 1.51 (m, 2H), 1.53-1.77 (m, 2H), 2.63-2.77 (m, 4H), 2.93-3.09 (m, 4H), 3.81-3.87 (m, 4H), 7.26 (d, J = 5.4 Hz, 1H), 7.36 (d, J = 8.1 Hz, 2H), 7.99 (d, J = 7.8 Hz, 2H), 8.29 (d, J = 5.4 Hz, 1H), 8.76 (s, 1H), 9.54 (s, 1H). |
| 74 | 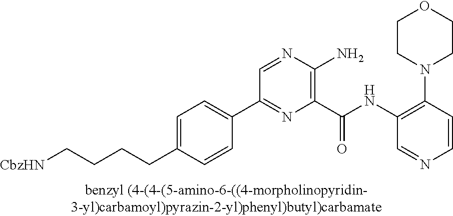 [SMILES: Nc1ncc(-c2ccc(CCCCNC(=O)OCc3ccccc3)cc2)nc1C(=O)Nc1cnccc1N1CCOCC1] | ¹H NMR (300 MHz, DMSO-d₆) δ: 1.24- 1.45 (m, 2H), 1.48-1.61 (m, 2H), 2.63-2.68 (m, 2H), 2.97-3.08 (m, 6H), 3.73 (s, 4H), 5.01 (s, 2H), 7.24-7.35 (m, 9H), 7.73 (s, 2H), 8.06 (d, J = 8.1 Hz, 2H), 8.32 (d, J = 5.4 Hz, 1H), 8.95 (s, 1H), 9.34 (s, 1H), 10.46 (s, 1H). |
| 75 | 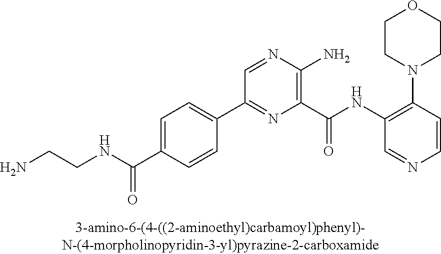 [SMILES: NCCNC(=O)c1ccc(-c2cnc(N)c(C(=O)Nc3cnccc3N3CCOCC3)n2)cc1] | ¹H NMR (300 MHz, DMSO-d₆) δ: 3.02- 3.06 (m, 2H), 3.47-3.69 (m, 6H), 3.70 (s, 4H), 7.43 (d, J = 6.0 Hz, 1H), 7.82-7.93 (m, 4H), 8.02 (d, J = 8.4 Hz, 2H), 8.33-8.38 (m, 3H), 8.75 (m, 1H), 8.87 (s, 1H), 9.08 (s, 1H), 10.50 (s, 1H). |
| 76 | 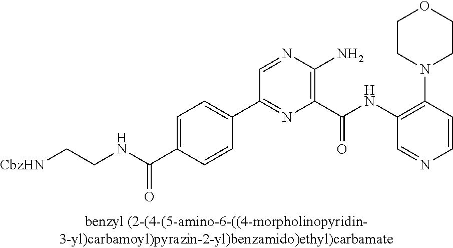 [SMILES: Nc1ncc(-c2ccc(C(=O)NCCNC(=O)OCc3ccccc3)cc2)nc1C(=O)Nc1cnccc1N1CCOCC1] | ¹H NMR (300 MHz, Methanol-d₄) δ: 3.04- 3.07 (m, 4H), 3.41-3.43 (m, 2H), 3.53-3.57 (m, 2H), 3.81-3.84 (m, 4H), 5.10 (s, 2H), 7.27-7.34 (m, 6H), 8.00 (d, J = 8.4 Hz, 2H), 8.20 (d, J = 8.4 Hz, 2H), 8.31 (d, J = 5.7 Hz, 1H), 8.87 (s, 1H), 9.46 (s, 1H). |
| 77 | 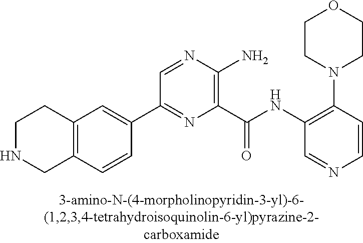 [SMILES: Nc1ncc(-c2ccc3c(c2)CCNC3)nc1C(=O)Nc1cnccc1N1CCOCC1] | ¹H NMR (300 MHz, Methanol-d₄) δ: 3.10- 3.29 (m, 2H), 3.53-3.57 (m, 6H), 3.81-3.84 (m, 4H), 4.46 (s, 2H), 7.40 (d, J = 8.7 Hz, 1H), 7.49 (d, J = 6.9 Hz, 1H), 7.99-8.04 (m, 2H), 8.35-8.37 (m, 1H), 8.87 (s, 1H), 9.11 (s, 1H). |
| 78 | 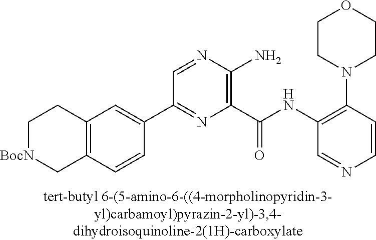 [SMILES: CC(C)(C)OC(=O)N1CCc2cc(-c3cnc(N)c(C(=O)Nc4cnccc4N4CCOCC4)n3)ccc2C1] | ¹H NMR (300 MHz, DMSO-d₆) δ: 1.45 (s, 9H), 2.88-2.98 (m, 6H), 3.61 (t, J = 6.0 Hz, 2H), 3.71-3.74 (m, 4H), 4.57 (s, 2H), 7.23 (d, J = 5.4 Hz, 1H), 7.32 (d, J = 8.1 Hz, 1H), 7.76 (s, 2H), 7.94-7.99 (m, 2H), 8.32 (d, J = 5.4 Hz, 1H), 8.95 (s, 1H), 9.32 (s, 1H), 10.36 (s, 1H). |
| 79 | 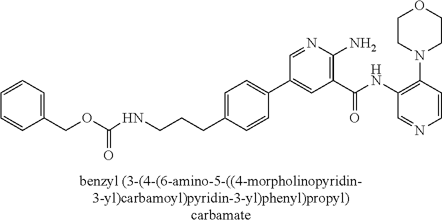 [SMILES: Nc1ncc(-c2ccc(CCCNC(=O)OCc3ccccc3)cc2)cc1C(=O)Nc1cnccc1N1CCOCC1] | ¹H NMR (400 MHz, Acetonitrile-d₃) δ: 1.79-1.85 (m, 2H), 2.66-2.70 (m, 2H), 3.03-3.06 (m, 4H), 3.13-3.18 (m, 2H), 3.73-3.78 (m, 4H), 5.07 (s, 2H), 5.71 (s, 1H), 6.63 (s, 2H), 7.07 (d, J = 5.2 Hz, 1H), 7.32-7.41 (m, 7H), 7.57-7.59 (m, 2H), 8.17 (d, J = 2.4 Hz, 1H), 8.32 (d, J = 5.6 Hz, 1H), 8.50 (d, J = 2.4 Hz, 1H), 8.74 (s, 1H), 8.95 (s, 1H). |
| 80 | 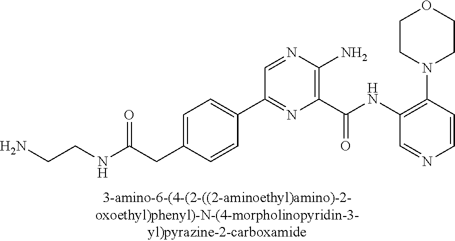 [SMILES: NCCNC(=O)Cc1ccc(-c2cnc(N)c(C(=O)Nc3cnccc3N3CCOCC3)n2)cc1] | ¹H NMR (300 MHz, DMSO-d₆) δ: 2.84- 2.90 (m, 2H), 3.30-3.35 (m, 2H), 3.44-3.68 (m, 6H), 3.70 (s, 4H), 7.40 (d, J = 7.2 Hz, 3H), 7.69-7.91 (m, 4H), 8.12-8.15 (m, 2H), 8.27-8.29 (m, 1H), 8.39 (d, J = 6.3 Hz, 1H), 8.87 (s, 1H), 8.96 (s, 1H), 10.50 (s, 1H). |
| 81 | 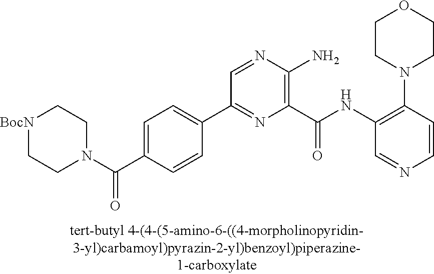 [SMILES: CC(C)(C)OC(=O)N1CCN(C(=O)c2ccc(-c3cnc(N)c(C(=O)Nc4cnccc4N4CCOCC4)n3)cc2)CC1] | ¹H NMR (400 MHz, DMSO-d₆) δ: 1.42 (s, 9H), 2.98 (s, 4H), 3.53-3.60 (m, 6H), 3.64- 3.69 (m, 2H), 3.70-3.71 (m, 4H), 7.24 (d, J = 5.2 Hz, 1H), 7.55 (d, J = 8.0 Hz, 2H), 7.83 (s, 2H), 8.21 (d, J = 8.4 Hz, 2H), 8.32 (d, J = 5.2 Hz, 1H), 9.01 (s, 1H), 9.33 (s, 1H), 10.47 (s, 1H). |
| 82 | 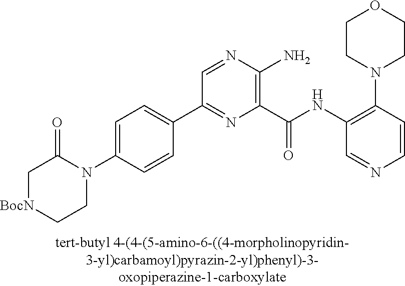 [SMILES: CC(C)(C)OC(=O)N1CCN(c2ccc(-c3cnc(N)c(C(=O)Nc4cnccc4N4CCOCC4)n3)cc2)C(=O)C1] | ¹H NMR (400 MHz, DMSO-d₆) δ: 1.47 (s, 9H), 2.99 (s, 4H), 3.69-3.81 (m, 8H), 4.11 (s, 2H), 7.24 (s, 1H), 7.52 (s, 2H), 7.79 (s, 2H), 8.20 (s, 2H), 8.33 (s, 1H), 9.28 (s, 1H), 9.37 (s, 1H), 10.41 (s, 1H). |
| 83 | 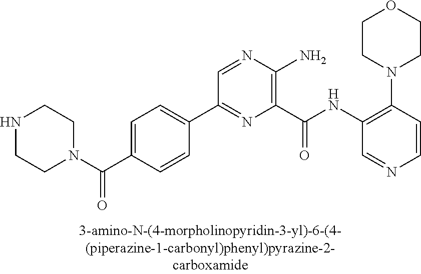 [SMILES: Nc1ncc(-c2ccc(C(=O)N3CCNCC3)cc2)nc1C(=O)Nc1cnccc1N1CCOCC1] | ¹H NMR (400 MHz, DMSO-d₆) δ: 1.24 (s, 1H), 2.68-2.72 (s, 4H), 2.97 (s, 4H), 3.41- 3.60 (m, 4H), 3.71 (s, 4H), 7.24 (d, J = 4.2 Hz, 1H), 7.52 (d, J = 8.4 Hz, 2H), 7.82 (s, 2H), 8.19-8.20 (m, 2H), 8.32-8.33 (m, 1H), 9.00 (s, 1H), 9.33 (s, 1H), 10.49 (s, 1H). |
| 84 | 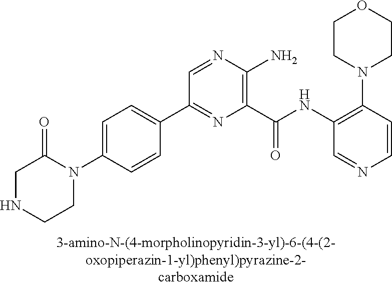 [SMILES: Nc1ncc(-c2ccc(N3CCNCC3=O)cc2)nc1C(=O)Nc1cnccc1N1CCOCC1] | ¹H NMR (400 MHz, Methanol-d₄) δ: 3.72- 3.74 (m, 4H), 3.80-3.82 (m, 2H), 3.88-3.97 (m, 4H), 4.04-4.06 (m, 4H), 7.46 (d, J = 6.4 Hz, 3H), 8.18 (d, J = 8.4 Hz, 2H), 8.33 (d, J = 6.8 Hz, 1H), 8.86 (s, 1H), 9.07 (s, 1H). |
| 85 | 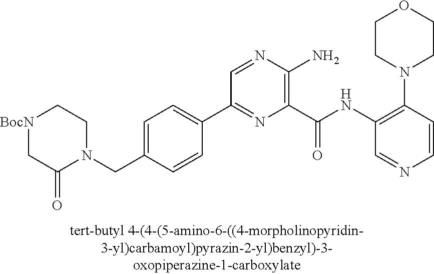 [SMILES: CC(C)(C)OC(=O)N1CCN(Cc2ccc(-c3cnc(N)c(C(=O)Nc4cnccc4N4CCOCC4)n3)cc2)C(=O)C1] | ¹H NMR (400 MHz, DMSO-d₆) δ: 1.41 (s, 9H), 2.96 (s, 4H), 3.28-3.30 (m, 4H), 3.54 (s, 2H), 3.70 (s, 4H), 4.63 (s, 2H), 7.24 (d, J = 5.6 Hz, 1H), 7.40 (d, J = 7.2 Hz, 2H), 7.75 (s, 2H), 8.11 (d, J = 8.0 Hz, 2H), 8.32 (d, J = 4.8 Hz, 1H), 8.95 (s, 1H), 9.32 (s, 1H), 10.46 (s, 1H). |
| 86 | 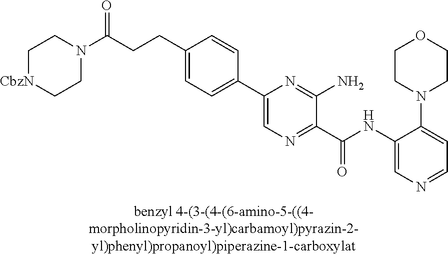 [SMILES: Nc1nc(-c2ccc(CCC(=O)N3CCN(C(=O)OCc4ccccc4)CC3)cc2)cnc1C(=O)Nc1cnccc1N1CCOCC1] | ¹H NMR (400 MHz, DMSO-d₆) δ: 2.69- 2.71 (m, 2H), 2.88-2.90 (m, 2H), 2.92-2.96 (m, 4H), 3.31-3.36 (m, 4H), 3.45-3.46 (m, 4H), 3.71-3.73 (m, 4H), 5.08 (s, 2H), 7.22 (d, J = 8.0 Hz, 1H), 7.30-7.40 (m, 7H), 7.72 (s, 2H), 8.07 (d, J = 8.0 Hz, 2H), 8.32 (d, J = 5.6 Hz, 1H), 8.95 (s, 1H), 9.35 (s, 1H), 10.43 (s, 1H). |
| 87 | 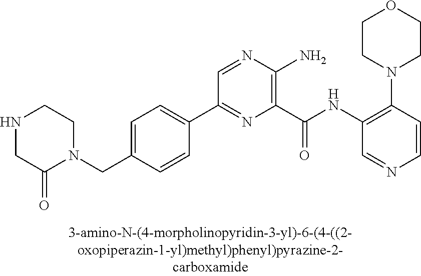 [SMILES: Nc1ncc(-c2ccc(CN3CCNCC3=O)cc2)nc1C(=O)Nc1cnccc1N1CCOCC1] | ¹H NMR (400 MHz, DMSO-d₆) δ: 3.42- 3.62 (m, 8H), 3.69 (m, 4H), 3.85 (s, 2H), 4.65 (s, 2H), 7.42-7.51 (m, 3H), 7.71 (s, 2H), 8.18 (d, J = 8.0 Hz, 2H), 8.39 (d, J = 6.8 Hz, 1H), 8.80 (s, 1H), 8.98 (s, 1H), 9.32 (s, 1H), 10.47 (s, 1H). |
| 88 | 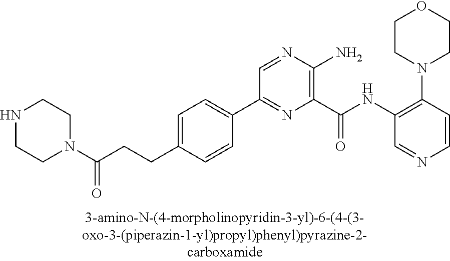 [SMILES: Nc1ncc(-c2ccc(CCC(=O)N3CCNCC3)cc2)nc1C(=O)Nc1cnccc1N1CCOCC1] | ¹H NMR (400 MHz, Methanol-d₄) δ: 2.69- 2.70 (m, 2H), 2.77-2.99 (m, 4H), 3.04-3.33 (m, 6H), 3.44-3.47 (m, 2H), 3.56-3.58 (m, 2H), 3.78-3.89 (m, 4H), 7.27 (d, J = 5.6 Hz, 1H), 7.41 (d, J = 8.4 Hz, 2H), 8.01 (d, J = 8.0 Hz, 2H), 8.28 (d, J = 5.2 Hz, 1H), 8.77 (s, 1H), 9.48 (s, 1H). |
| 89 | 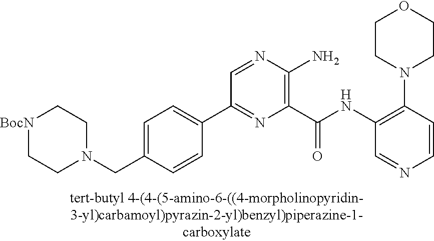 [SMILES: CC(C)(C)OC(=O)N1CCN(Cc2ccc(-c3cnc(N)c(C(=O)Nc4cnccc4N4CCOCC4)n3)cc2)CC1] | ¹H NMR (300 MHz, DMSO-d₆) δ: 1.40 (s, 9H), 2.35 (s, 4H), 2.97 (s, 4H), 3.33 (s, 4H), 3.57 (s, 2H), 3.71 (s, 4H), 7.25 (d, J = 6.0 Hz, 1H), 7.45 (d, J = 6.0 Hz, 2H), 7.75 (s, 2H), 8.09 (d, J = 6.0 Hz, 2H), 8.32 (d, J = 6.0 Hz, 1H), 8.95 (s, 1H), 9.36 (s, 1H), 10.49 (s, 1H). |
| 90 | 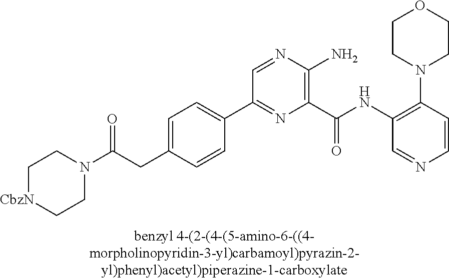 [SMILES: Nc1ncc(-c2ccc(CC(=O)N3CCN(C(=O)OCc4ccccc4)CC3)cc2)nc1C(=O)Nc1cnccc1N1CCOCC1] | ¹H NMR (300 MHz, DMSO-d₆) δ: 3.00 (s, 4H), 3.30-3.37 (m, 4H), 3.51 (s, 4H), 3.70 (s, 4H), 3.83 (s, 2H), 5.07 (s, 2H), 7.24 (d, J = 2.7 Hz, 1H), 7.30-7.40 (m, 7H), 7.75 (s, 2H), 8.10 (d, J = 4.0 Hz, 2H), 8.32 (d, J = 2.7 Hz, 1H), 9.00 (s, 1H), 9.36 (s, 1H), 10.46 (s, 1H). |
| 91 | 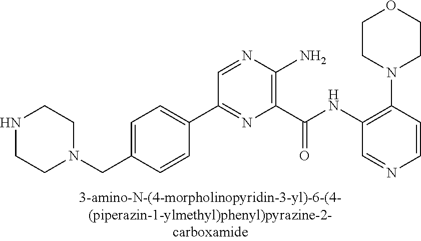 [SMILES: Nc1ncc(-c2ccc(CN3CCNCC3)cc2)nc1C(=O)Nc1cnccc1N1CCOCC1] | ¹H NMR (300 MHz, Methanol-d₄) δ: 3.51- 3.57 (m, 12H), 3.78-3.81 (m, 4H), 4.45 (s, 2H), 7.53 (d, J = 6.9 Hz, 1H), 7.71 (d, J = 8.1 Hz, 2H), 8.19 (d, J = 8.1 Hz, 2H), 8.36- 8.39 (m, 1H), 8.89 (s, 1H), 9.11 (s, 1H). |
| 92 | 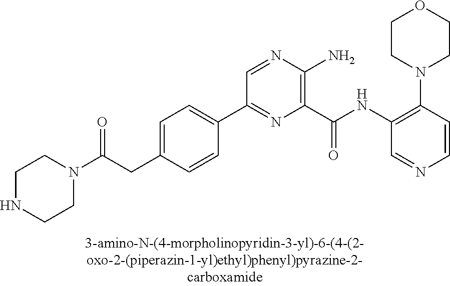 [SMILES: Nc1ncc(-c2ccc(CC(=O)N3CCNCC3)cc2)nc1C(=O)Nc1cnccc1N1CCOCC1] | ¹H NMR (300 MHz, Methanol-d₄) δ: 2.65 (t, J = 5.0 Hz, 2H), 2.79 (t, J = 5.0 Hz, 2H), 3.04 (t, J = 4.5 Hz, 4H), 3.54 (t, J = 5.0 Hz, 2H), 3.64 (t, J = 5.0 Hz, 4H), 3.82 (t, J = 4.5 Hz, 2H), 3.90 (s, 2H), 7.29 (d, J = 5.4 Hz, 1H), 7.46 (d, J = 8.1 Hz, 2H), 8.07 (d, J = 8.1 Hz, 2H), 8.29 (d, J = 5.4 Hz, 1H), 8.80 (s, 1H), 9.49 (s, 1H). |
| 93 | 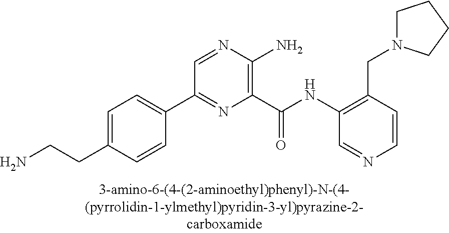 [SMILES: NCCc1ccc(-c2cnc(N)c(C(=O)Nc3cnccc3CN3CCCC3)n2)cc1] | ¹H NMR (300 MHz, Methanol-d₄) δ: 2.10 (s, 4H), 3.02-3.07 (m, 2H), 3.21-3.26 (m, 2H), 3.62 (s, 4H), 4.45 (s, 2H), 7.40-7.45 (m, 2H), 7.80 (d, J = 6.9 Hz, 1H), 8.15 (d, J = 8.1 Hz, 2H), 8.66 (s, 1H), 8.84 (s, 2H). |
| 94 | 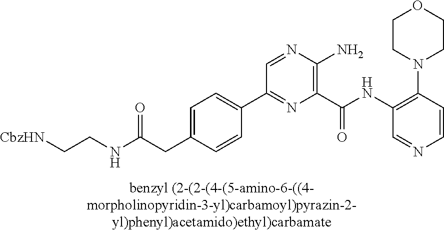 [SMILES: Nc1ncc(-c2ccc(CC(=O)NCCNC(=O)OCc3ccccc3)cc2)nc1C(=O)Nc1cnccc1N1CCOCC1] | ¹H NMR (400 MHz, Methanol-d₄) δ: 2.99 (s, 4H), 3.27-3.32 (m, 4H), 3.58 (s, 2H), 3.72-3.73 (m, 4H), 5.03 (s, 2H), 7.23-7.34 (m, 6H), 7.46 (d, J = 8.0 Hz, 2H), 8.01 (d, J = 8.0 Hz, 2H), 8.28 (d, J = 5.6 Hz, 1H), 8.74 (s, 1H), 9.46 (s, 1H). |
| 95 | 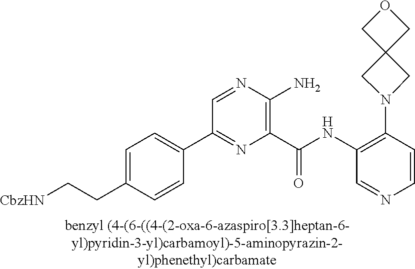 [SMILES: Nc1ncc(-c2ccc(CCNC(=O)OCc3ccccc3)cc2)nc1C(=O)Nc1cnccc1N1CC2(COC2)C1] | ¹H NMR (400 MHz, Methanol-d₄) δ: 2.85- 2.88 (m, 2H), 3.32-3.49 (m, 2H), 4.30 (s, 4H), 4.69-4.74 (m, 4H), 5.07-5.14 (m, 2H), 6.54 (d, J = 5.6 Hz, 1H), 7.27-7.59 (m, 7H), 8.00-8.08 (m, 3H), 8.32 (s, 1H), 8.78 (s, 1H). |
| 96 | 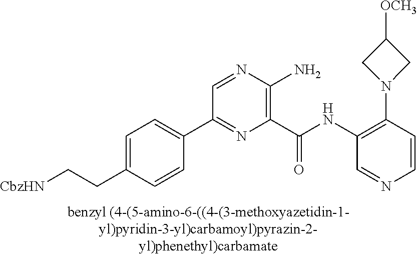 [SMILES: COC1CN(c2ccncc2NC(=O)c2nc(-c3ccc(CCNC(=O)OCc4ccccc4)cc3)cnc2N)C1] | ¹H NMR (400 MHz, Methanol-d₄) δ: 2.85- 2.90 (m, 2H), 3.24 (s, 3H), 3.39-3.44 (m, 2H), 3.98-4.01 (m, 2H), 4.30-4.37 (m, 3H), 5.08 (s, 2H), 6.55 (d, J = 7.6 Hz, 1H), 7.33- 7.38 (m, 7H), 8.00-8.09 (m, 3H), 8.27 (s, 1H), 8.78 (s, 1H). |
| 97 | 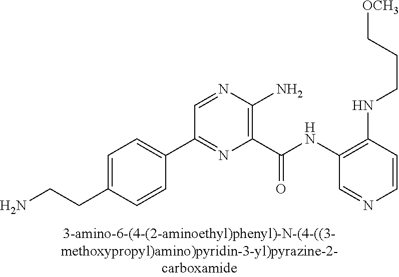 [SMILES: COCCCNc1ccncc1NC(=O)c1nc(-c2ccc(CCN)cc2)cnc1N] | ¹H NMR (400 MHz, Methanol-d₄) δ: 1.87- 1.89 (m, 2H), 2.79-2.86 (m, 2H), 2.90-2.97 (m, 2H), 3.15 (s, 3H), 3.22-3.34 (m, 2H), 3.36-3.52 (m, 2H), 6.75 (d, J = 6.0 Hz, 1H), 7.29-7.37 (m, 2H), 8.01-8.08 (m, 3H), 8.14 (s, 1H), 8.77 (s, 1H). |
| 98 | 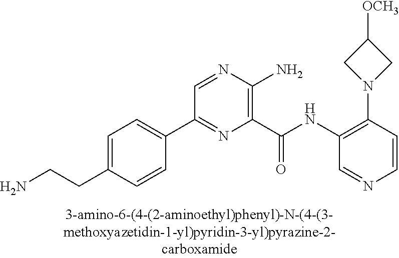 [SMILES: COC1CN(c2ccncc2NC(=O)c2nc(-c3ccc(CCN)cc3)cnc2N)C1] | ¹H NMR (400 MHz, Methanol-d₄) δ: 2.91- 3.15 (m, 2H), 3.21-3.33 (m, 2H), 3.45-3.50 (m, 3H), 4.31-4.38 (m, 3H), 4.62-4.66 (m, 2H), 6.76 (d, J = 7.2 Hz, 1H), 7.43 (d, J = 8.4 Hz, 2H), 8.09-8.15 (m, 3H), 8.32 (d, J = 1.2 Hz, 1H), 8.83 (s, 1H). |
| 99 | 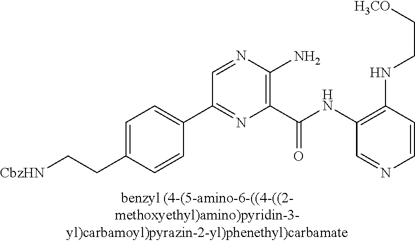 [SMILES: COCCNc1ccncc1NC(=O)c1nc(-c2ccc(CCNC(=O)OCc3ccccc3)cc2)cnc1N] | ¹H NMR (300 MHz, Methanol-d₄) δ: 2.84- 2.88 (m, 2H), 3.32-3.42 (m, 5H), 3.63 (s, 4H), 5.10 (s, 2H), 7.22 (d, J = 7.2 Hz, 1H), 7.26-7.37 (m, 7H), 8.07 (d, J = 8.1 Hz, 2H), 8.29 (dd, J₁ = 7.2 Hz, J₂ = 1.2 Hz, 1H), 8.30 (s, 1H), 8.81 (s, 1H). |
| 100 | 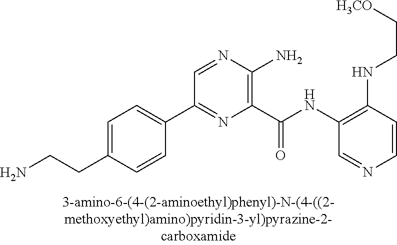 [SMILES: COCCNc1ccncc1NC(=O)c1nc(-c2ccc(CCN)cc2)cnc1N] | ¹H NMR (300 MHz, Methanol-d₄) δ: 2.85- 2.87 (m, 2H), 2.90-3.02 (m, 2H), 3.33-3.35 (m, 3H), 3.44 (t, J = 5.7 Hz, 2H), 3.61 (t, J = 5.7 Hz, 2H), 6.82 (d, J = 5.7 Hz, 1H), 7.38 (d, J = 8.4 Hz, 2H), 8.06-8.10 (m, 3H), 8.16 (s, 1H), 8.77 (s, 1H). |
| 101 | 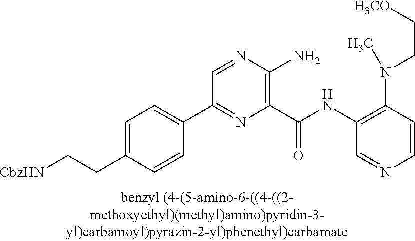 [SMILES: COCCN(C)c1ccncc1NC(=O)c1nc(-c2ccc(CCNC(=O)OCc3ccccc3)cc2)cnc1N] | ¹H NMR (300 MHz, DMSO-d₆) δ: 2.77- 2.81 (m, 2H), 2.88 (s, 3H), 3.02 (s, 3H), 3.25-3.33 (m, 4H), 3.45-3.49 (m, 2H), 5.02 (s, 2H), 7.06 (d, J = 5.4 Hz, 1H), 7.29-7.57 (m, 8H), 7.67 (s, 2H), 8.10 (d, J = 8.1 Hz, 2H), 8.21 (d, J = 5.4 Hz, 1H), 8.74 (s, 1H), 8.94 (s, 1H), 10.38 (s, 1H). |
| 102 | 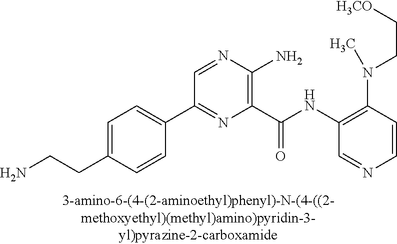 [SMILES: COCCN(C)c1ccncc1NC(=O)c1nc(-c2ccc(CCN)cc2)cnc1N] | ¹H NMR (300 MHz, Acetonitrile-d₃ + D₂O) δ: 2.78-2.75 (m, 2H), 2.88-2.93 (m, 5H), 3.10 (s, 3H), 3.29-3.32 (m, 2H), 3.50-3.52 (m, 2H), 7.10 (d, J = 4.2 Hz, 1H), 7.37 (d, J = 6.0 Hz, 2H), 7.97 (d, J = 6.0 Hz, 2H), 8.25 (d, J = 4.2 Hz, 1H), 8.78 (s, 1H), 9.14 (s, 1H), 10.30 (s, 1H). |
| 103 | 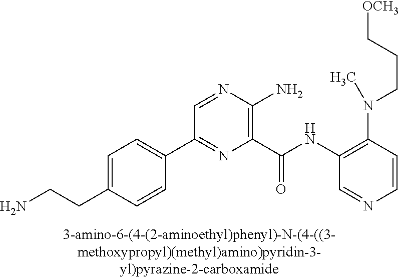 [SMILES: COCCCN(C)c1ccncc1NC(=O)c1nc(-c2ccc(CCN)cc2)cnc1N] | ¹H NMR (400 MHz, Methanol-d₄) δ: 1.81- 1.88 (m, 2H), 2.85-2.90 (m, 5H), 3.08-3.09 (s, 2H), 3.25-3.32 (m, 3H), 3.33-3.43 (m, 4H), 7.17 (d, J = 5.6 Hz, 1H), 7.39 (d, J = 8.4 Hz, 2H), 8.00 (d, J = 8.0 Hz, 2H), 8.21 (d, J = 5.6 Hz, 1H), 8.79 (s, 1H), 9.14 (s, 1H). |
| 104 | 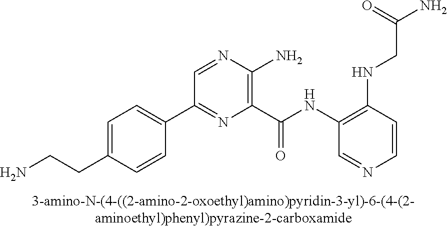 [SMILES: NCCc1ccc(-c2cnc(N)c(C(=O)Nc3cnccc3NCC(N)=O)n2)cc1] | ¹H NMR (400 MHz, Methanol-d₄) δ: 3.01- 3.05 (m, 2H), 3.21-3.23 (m, 2H), 4.16 (s, 2H), 7.00 (d, J = 7.2 Hz, 1H), 7.41-7.49 (m, 2H), 8.14-8.17 (m, 2H), 8.22-8.25 (m, 1H), 8.39 (s, 1H), 8.82 (s, 1H). |
| 105 | 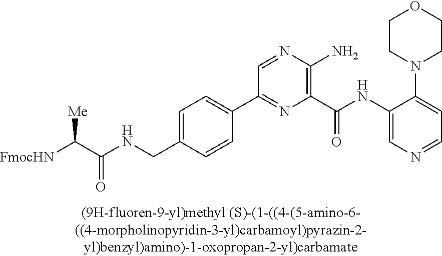 | [M + H]⁺: 699.4 |
| 106 | 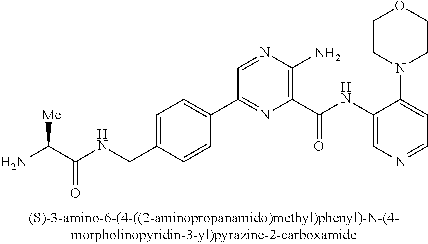 [SMILES: C[C@H](N)C(=O)NCc1ccc(-c2cnc(N)c(C(=O)Nc3cnccc3N3CCOCC3)n2)cc1] | [M + H]⁺: 477.3 |
| 107 | 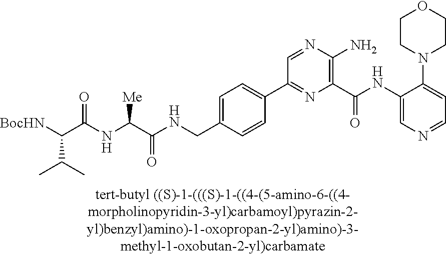 [SMILES: CC(C)[C@H](NC(=O)OC(C)(C)C)C(=O)N[C@@H](C)C(=O)NCc1ccc(-c2cnc(N)c(C(=O)Nc3cnccc3N3CCOCC3)n2)cc1] | [M + H]⁺: 676.4 |
| 108 | 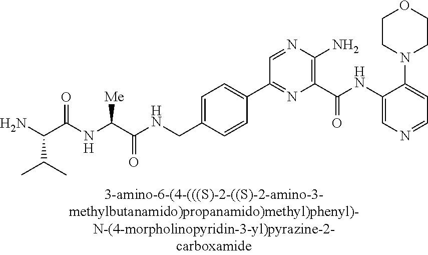 [SMILES: CC(C)[C@H](N)C(=O)N[C@@H](C)C(=O)NCc1ccc(-c2cnc(N)c(C(=O)Nc3cnccc3N3CCOCC3)n2)cc1] | [M + H]⁺: 576.4 |
| 109 | 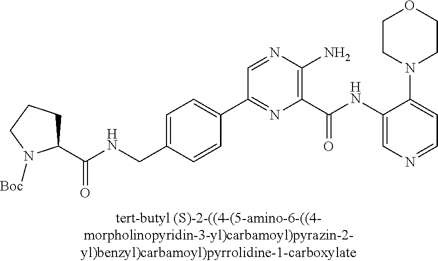 [SMILES: CC(C)(C)OC(=O)N1CCC[C@H]1C(=O)NCc1ccc(-c2cnc(N)c(C(=O)Nc3cnccc3N3CCOCC3)n2)cc1] | [M + H]⁺: 603.4 |
| 110 | 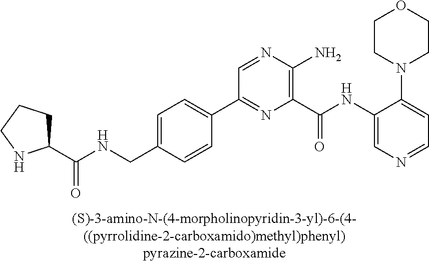 [SMILES: Nc1ncc(-c2ccc(CNC(=O)[C@@H]3CCCN3)cc2)nc1C(=O)Nc1cnccc1N1CCOCC1] | [M + H]⁺: 503.3 |
| 111 | 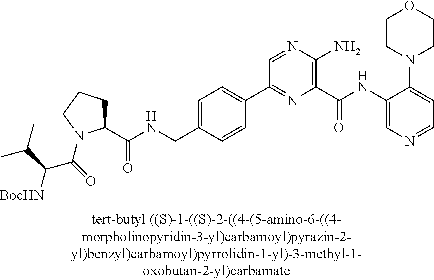 [SMILES: CC(C)[C@H](NC(=O)OC(C)(C)C)C(=O)N1CCC[C@H]1C(=O)NCc1ccc(-c2cnc(N)c(C(=O)Nc3cnccc3N3CCOCC3)n2)cc1] | [M + H]⁺: 702.4 |
| 112 | 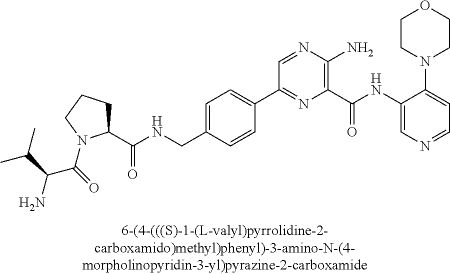 [SMILES: CC(C)[C@H](N)C(=O)N1CCC[C@H]1C(=O)NCc1ccc(-c2cnc(N)c(C(=O)Nc3cnccc3N3CCOCC3)n2)cc1] | [M + H]⁺: 602.4 |
| 113 | 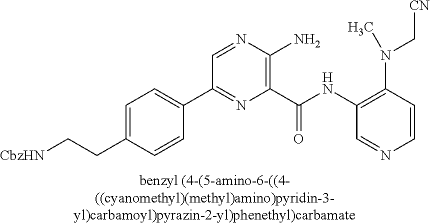 [SMILES: CN(CC#N)c1ccncc1NC(=O)c1nc(-c2ccc(CCNC(=O)OCc3ccccc3)cc2)cnc1N] | ¹H NMR (300 MHz, Chloroform-d₁) δ: 2.64-2.90 (m, 5H), 3.37-3.47 (m, 2H), 4.75 (s, 2H), 4.97 (s, 1H), 5.23 (s, 2H), 6.34 (s, 1H), 7.50-7.54 (m, 8H), 7.73 (d, J = 7.0 Hz, 2H), 7.99-8.34 (m, 2H), 8.53 (s, 1H), 10.13 (s, 1H). (one active hydrogen was not found) |
| 114 | 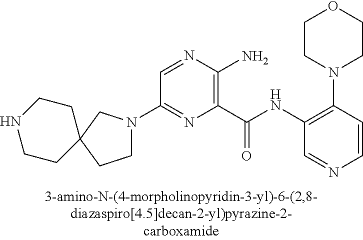 [SMILES: Nc1ncc(N2CCC3(CCNCC3)C2)nc1C(=O)Nc1cnccc1N1CCOCC1] | ¹H NMR (400 MHz, Methanol-d₄) δ: 1.41 (s, 4H), 2.00-2.03 (m, 2H), 2.82-2.96 (m, 4H), 3.02-3.07 (m, 4H), 3.49 (s, 2H), 3.63- 3.67 (m, 2H), 3.90-3.92 (m, 4H), 7.24 (d, J = 5.6 Hz, 1H), 7.91 (s, 1H), 8.26 (d, J = 5.6 Hz, 1H), 9.31 (s, 1H). |
| 115 | 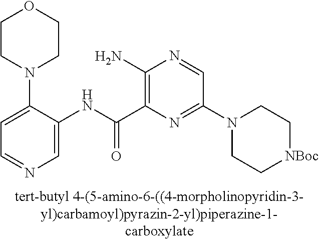 [SMILES: CC(C)(C)OC(=O)N1CCN(c2cnc(N)c(C(=O)Nc3cnccc3N3CCOCC3)n2)CC1] | ¹H NMR (300 MHz, Methanol-d₄) δ: 1.52 (s, 9H), 3.05-3.08 (m, 4H), 3.50-3.59 (m, 4H), 3.64-3.67 (m, 4H), 3.89-3.92 (m, 4H), 7.26 (d, J = 5.4 Hz, 1H), 8.20 (s, 1H), 8.28 (d, J = 5.4 Hz, 1H), 9.34 (s, 1H). |
| 116 | 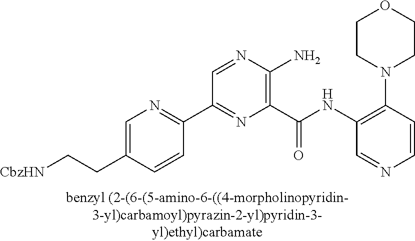 [SMILES: Nc1ncc(-c2ccc(CCNC(=O)OCc3ccccc3)cn2)nc1C(=O)Nc1cnccc1N1CCOCC1] | ¹H NMR (400 MHz, Methanol-d₄) δ: 2.91- 2.94 (m, 2H), 3.14-3.15 (m, 4H), 3.50-3.57 (m, 2H), 3.77-3.85 (m, 4H), 5.06 (s, 2H), 7.20-7.25 (m, 2H), 7.26-7.32 (m, 4H), 7.84-7.86 (m, 1H), 8.21-8.31 (m, 2H), 8.53 (s, 1H), 9.14 (s, 1H), 9.37 (s, 1H). |
| 117 | 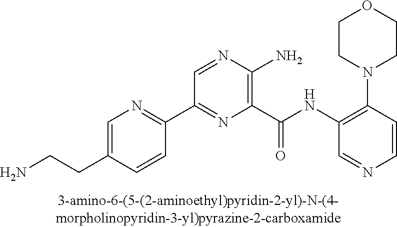 [SMILES: NCCc1ccc(-c2cnc(N)c(C(=O)Nc3cnccc3N3CCOCC3)n2)nc1] | ¹H NMR (400 MHz, Methanol-d₄) δ: 3.21- 3.25 (m, 2H), 3.32-3.38 (m, 2H), 3.69-3.78 (m, 8H), 7.42 (d, J = 7.2 Hz, 1H), 8.28 (d, J = 6.8 Hz, 1H), 8.45 (d, J = 8.4 Hz, 1H), 8.60-8.64 (m, 2H), 9.03 (s, 1H), 9.24 (s, 1H). |
| 118 | 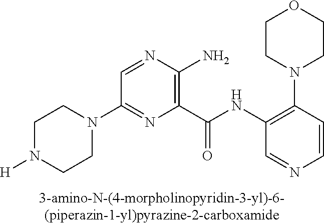 [SMILES: Nc1ncc(N2CCNCC2)nc1C(=O)Nc1cnccc1N1CCOCC1] | ¹H NMR (300 MHz, Methanol-d₄) δ: 3.38- 3.51 (m, 4H), 3.59-3.69 (m, 4H), 3.75-3.85 (m, 8H), 7.41 (d, J = 6.9 Hz, 1H), 8.26-8.28 (m, 2H), 8.71 (s, 1H). |
| 119 | 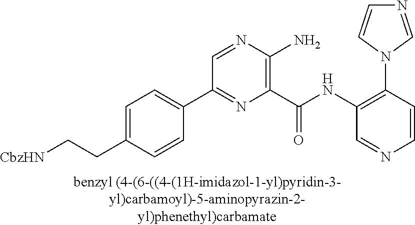 [SMILES: Nc1ncc(-c2ccc(CCNC(=O)OCc3ccccc3)cc2)nc1C(=O)Nc1cnccc1-n1ccnc1] | [M + H]⁺: 609.3 |
| 120 | 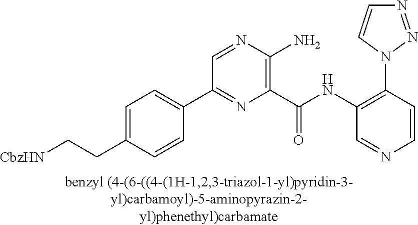 [SMILES: Nc1ncc(-c2ccc(CCNC(=O)OCc3ccccc3)cc2)nc1C(=O)Nc1cnccc1-n1ccnn1] | [M + H]⁺: 536.3 |
| 121 | 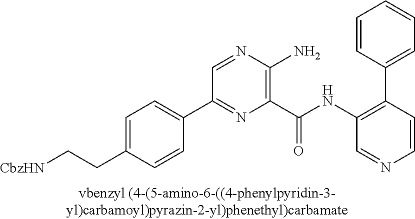 [SMILES: Nc1ncc(-c2ccc(CCNC(=O)OCc3ccccc3)cc2)nc1C(=O)Nc1cnccc1-c1ccccc1] | [M + H]⁺: 545.3 |
| 122 | 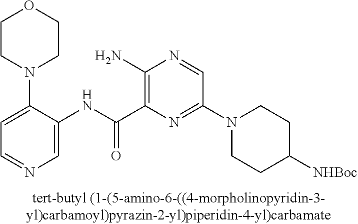 [SMILES: CC(C)(C)OC(=O)NC1CCN(c2cnc(N)c(C(=O)Nc3cnccc3N3CCOCC3)n2)CC1] | ¹H NMR (400 MHz, Methanol-d₄) δ: 1.47 (s, 9H), 1.56-1.64 (m, 2H), 2.03 (d, J = 11.2 Hz, 2H), 3.01-3.06 (m, 6H), 3.60 (s, 1H), 3.88-3.90 (m, 4H), 4.18-4.22 (m, 2H), 7.24-7.32 (m, 1H), 8.17 (s, 1H), 8.26 (d, J = 6.8 Hz, 1H), 9.36 (s, 1H). |
| 123 | 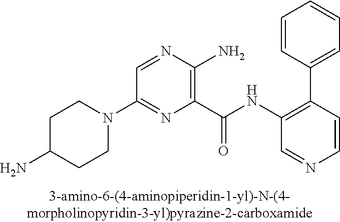 [SMILES: Nc1ncc(N2CCC(N)CC2)nc1C(=O)Nc1cnccc1-c1ccccc1] | ¹H NMR (300 MHz, Methanol-d₄) δ: 1.66- 1.79 (m, 2H), 2.15 (d, J = 9.9 Hz, 2H), 2.93-3.02 (m, 2H), 3.36-3.42 (m, 1H), 3.57-3.62 (m, 4H), 3.81-3.86 (m, 4H), 4.34 (d, J = 13.2 Hz, 2H), 7.45 (d, J = 6.9 Hz, 1H), 8.25-8.31 (m, 2H), 8.84 (d, J = 1.2 Hz, 1H). |
| 124 | 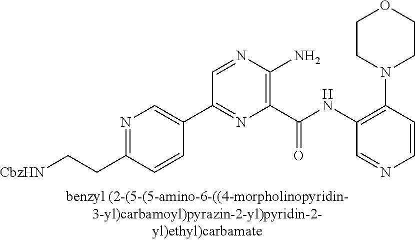 [SMILES: Nc1ncc(-c2ccc(CCNC(=O)OCc3ccccc3)nc2)nc1C(=O)Nc1cnccc1N1CCOCC1] | ¹H NMR (300 MHz, DMSO-d₆ + D₂O) δ: 2.84-2.92 (m, 6H), 3.38 (s, 2H), 3.62 (s, 4H), 5.01 (s, 2H), 7.11- 7.55 (m, 7H), 8.24-8.33 (m, 2H), 8.85 (s, 1H), 9.13 (d, J = 4.8 Hz, 2H). |
| 125 | 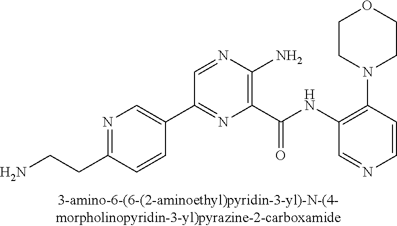 [SMILES: NCCc1ccc(-c2cnc(N)c(C(=O)Nc3cnccc3N3CCOCC3)n2)cn1] | ¹H NMR (400 MHz, Methanol-d₄) δ: 3.32- 3.37 (m, 2H), 3.45-3.48 (m, 2H), 3.63-3.70 (m, 4H), 3.79-3.81 (m, 4H), 7.47 (d, J = 6.8 Hz, 1H), 7.72 (d, J = 8 Hz, 1H), 8.32- 8.34 (m, 1H), 8.75-8.78 (m, 1H), 8.86 (s, 1H), 8.95 (s, 1H), 9.38 (d, J = 2.0 Hz, 1H). |
| 126 | 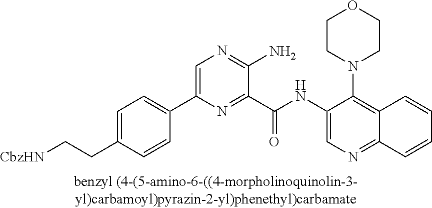 [SMILES: Nc1ncc(-c2ccc(CCNC(=O)OCc3ccccc3)cc2)nc1C(=O)Nc1cnc2ccccc2c1N1CCOCC1] | ¹H NMR (400 MHz, DMSO-d₆) δ: 2.78- 2.82 (m, 2H), 3.26-3.31 (m, 2H), 3.35-3.37 (m, 4H), 3.80 (s, 4H), 5.01 (s, 2H), 7.61- 7.64 (m, 8H), 7.70-7.81 (m, 4H), 7.99-8.04 (m, 1H), 8.16-8.20 (m, 3H), 8.22-8.95 (m, 1H), 9.10-9.25 (s, 1H), 10.68 (s, 1H). |
| 127 | 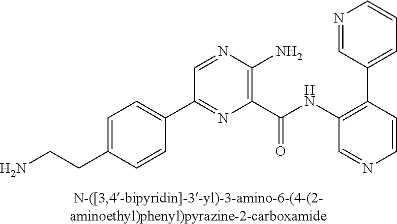 [SMILES: NCCc1ccc(-c2cnc(N)c(C(=O)Nc3cnccc3-c3cccnc3)n2)cc1] | ¹H NMR (300 MHz, Methanol-d₄) δ: 3.02- 3.07 (m, 2H), 3.24-3.29 (m, 2H), 7.35 (d, J = 8.1 Hz, 2H), 7.52-7.67 (m, 4H), 8.11 (d, J = 8.1 Hz, 1H), 8.52 (d, J = 5.1 Hz, 1H), 8.74-8.81 (m, 3H), 9.60 (s, 1H). |
| 128 | 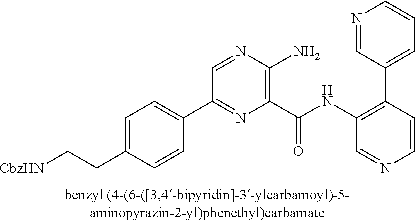 [SMILES: Nc1ncc(-c2ccc(CCNC(=O)OCc3ccccc3)cc2)nc1C(=O)Nc1cnccc1-c1cccnc1] | ¹H NMR (300 MHz, DMSO-d₆) δ: 2.77- 2.82 (m, 2H), 3.26-3.28 (m, 2H), 5.03 (s, 2H), 7.25-7.41 (m, 8H), 7.50-7.74 (m, 6H), 8.05 (m, 1H), 8.56 (d, J = 4.8 Hz, 1H), 8.73 (d, J = 3.3 Hz, 1H), 8.80 (s, 1H), 8.88 (s, 1H), 9.29 (s, 1H), 10.28 (s, 1H). |
| 129 | 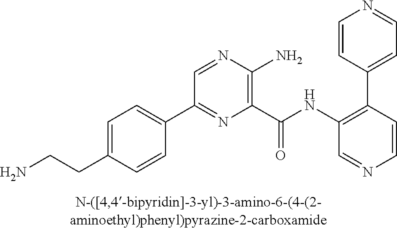 [SMILES: NCCc1ccc(-c2cnc(N)c(C(=O)Nc3cnccc3-c3ccncc3)n2)cc1] | ¹H NMR (400 MHz, Methanol-d₄) δ: 3.03- 3.07 (m, 2H), 3.22-3.30 (m, 2H), 7.37-7.39 (m, 2H), 7.63 (d, J = 8.0 Hz, 2H), 7.96-8.01 (m, 3H), 8.73 (d, J = 5.2 Hz, 1H), 8.79 (s, 1H), 8.80-8.92 (m, 2H), 9.83 (s, 1H). |
| 130 | 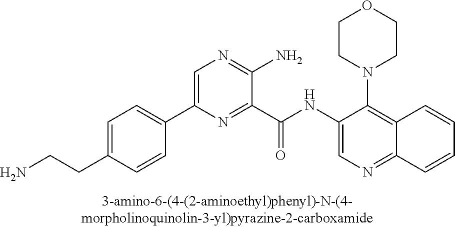 [SMILES: NCCc1ccc(-c2cnc(N)c(C(=O)Nc3cnc4ccccc4c3N3CCOCC3)n2)cc1] | ¹H NMR (400 MHz, Methanol-d₄) δ: 2.83- 2.96 (m, 2H), 2.98-2.99 (m, 2H), 3.49 (s, 4H), 3.88-3.90 (m, 4H), 7.38 (d, J = 8.4 Hz, 2H), 7.60-7.72 (m, 2H), 7.94-8.03 (m, 3H), 8.26-8.28 (m, 1H), 9.52 (s, 1H), 9.56 (s, 1H). |
| 131 | 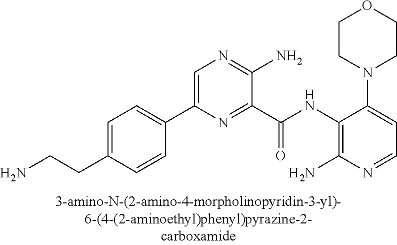 [SMILES: NCCc1ccc(-c2cnc(N)c(C(=O)Nc3c(N4CCOCC4)ccnc3N)n2)cc1] | ¹H NMR (300 MHz, Methanol-d₄) δ: 2.83- 2.89 (m, 2H), 3.00-3.10 (m, 6H), 3.66-3.69 (m, 4H), 6.56 (d, J = 7.5 Hz, 1H), 7.39 (d, J = 8.4 Hz, 2H), 7.87 (d, J = 7.5 Hz, 1H), 8.06-8.08 (m, 2H), 8.83 (s, 1H). |
| 132 | 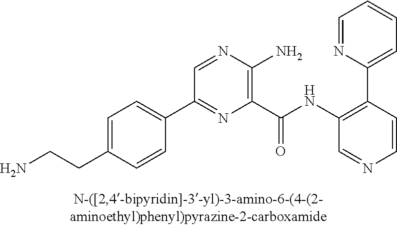 [SMILES: NCCc1ccc(-c2cnc(N)c(C(=O)Nc3cnccc3-c3ccccn3)n2)cc1] | ¹H NMR (400 MHz, Methanol-d₄) δ: 3.08- 3.28 (m, 4H), 7.47-7.52 (m, 3H), 8.01-8.19 (m, 5H), 8.56-8.64 (m, 2H), 8.77 (s, 1H), 10.03 (s, 1H). |
| 133 | 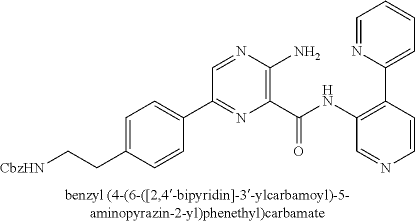 [SMILES: Nc1ncc(-c2ccc(CCNC(=O)OCc3ccccc3)cc2)nc1C(=O)Nc1cnccc1-c1ccccn1] | ¹H NMR (400 MHz, DMSO-d₆) δ: 2.49- 2.51 (m, 2H), 3.31-3.36 (m, 2H), 5.02 (s, 2H), 7.25-7.35 (m, 9H), 7.71 (s, 2H), 7.84 (s, 1H), 8.00-8.07 (m, 4H), 8.49 (d, J = 5.2 Hz, 1H), 8.59 (d, J = 4.8 Hz, 1H), 8.91 (s, 1H), 9.69 (s, 1H), 12.92 (s, 1H). |
| 134 | 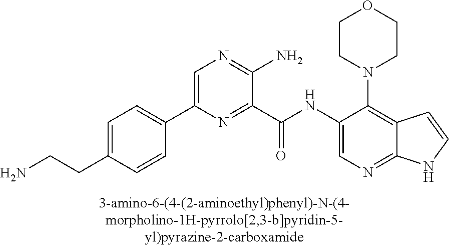 [SMILES: NCCc1ccc(-c2cnc(N)c(C(=O)Nc3cnc4[nH]ccc4c3N3CCOCC3)n2)cc1] | ¹H NMR (400 MHz, Methanol-d₄) δ: 2.88- 2.96 (m, 2H), 3.13-3.14 (m, 2H), 3.35 (s, 4H), 3.83-3.90 (m, 4H), 6.75 (s, 1H), 7.30- 7.34 (m, 3H), 8.03 (d, J = 8.0 Hz, 2H), 8.76 (s, 1H), 9.12 (s, 1H). |
| 135 | 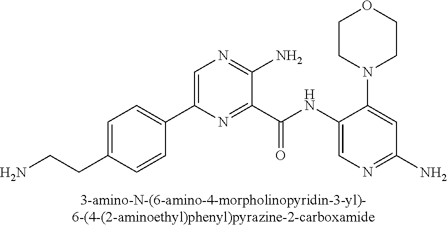 [SMILES: NCCc1ccc(-c2cnc(N)c(C(=O)Nc3cnc(N)cc3N3CCOCC3)n2)cc1] | ¹H NMR (300 MHz, DMSO-d₆ + D₂O) δ: 2.91-2.95 (m, 2H), 3.07-3.09 (m, 2H), 3.25-3.40 (m, 4H), 3.55-3.85 (m, 4H), 6.38 (s, 1H), 7.40 (d, J = 8.4 Hz, 2H), 8.15 (d, J = 8.1 Hz, 2H), 8.21 (s, 1H), 8.95 (s, 1H). |
| 136 | 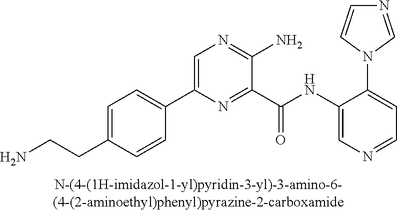 [SMILES: NCCc1ccc(-c2cnc(N)c(C(=O)Nc3cnccc3-n3ccnc3)n2)cc1] | ¹H NMR (400 MHz, Methanol-d₄) δ: 2.83- 2.94 (m, 2H), 3.07-3.13 (m, 2H), 7.30-7.32 (m, 1H), 7.55-7.59 (m, 2H), 7.67-7.69 (m, 2H), 7.75 (d, J = 8.0 Hz, 2H), 8.13 (s, 1H), 8.52 (d, J = 5.2 Hz, 1H), 8.75 (s, 1H), 9.63 (s, 1H). |
| 137 | 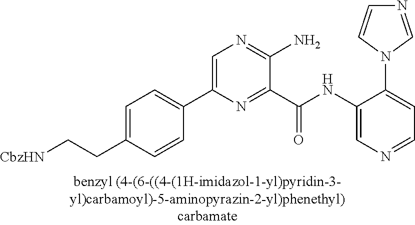 [SMILES: Nc1ncc(-c2ccc(CCNC(=O)OCc3ccccc3)cc2)nc1C(=O)Nc1cnccc1-n1ccnc1] | ¹H NMR (400 MHz, Methanol-d₄) δ: 2.84- 2.86 (m, 2H), 3.40-3.42 (m, 2H), 5.06 (s, 2H), 7.25-7.31 (m, 8H), 7.53-7.59 (m, 2H), 7.66-7.68 (m, 2H), 8.09 (s, 1H), 8.51 (d, J = 5.2 Hz, 1H), 8.70 (s, 1H), 9.60 (s, 1H). |
| 138 | 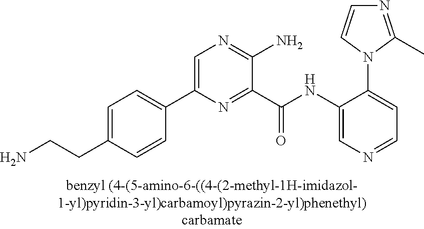 [SMILES: Cc1nccn1-c1ccncc1NC(=O)c1nc(-c2ccc(CCN)cc2)cnc1N] | ¹H NMR (400 MHz, Methanol-d₄) δ: 2.29 (s, 3H), 2.90-2.93 (m, 2H), 3.06-3.08 (m, 2H), 7.21 (s, 1H), 7.31-7.39 (m, 3H), 7.66 (s, 1H), 7.67 (d, J = 8.4 Hz, 2H), 8.53 (d, J = 5.2 Hz, 1H), 8.76 (s, 1H), 9.83 (s, 1H). |
| 139 | 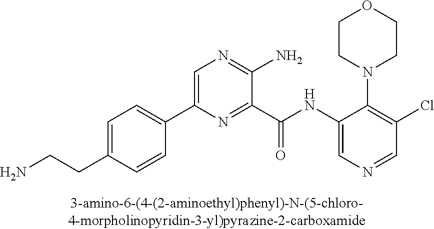 [SMILES: NCCc1ccc(-c2cnc(N)c(C(=O)Nc3cncc(Cl)c3N3CCOCC3)n2)cc1] | ¹H NMR (400 MHz, Methanol-d₄) δ: 2.84- 2.88 (m, 2H), 3.02-3.06 (m, 2H), 3.44-2.49 (m, 4H), 3.81-3.83 (m, 4H), 7.43 (d, J = 8.0 Hz, 2H), 7.95 (d, J = 8.0 Hz, 2H), 8.29 (s, 1H), 8.78 (s, 1H), 9.65 (s, 1H). |
| 140 | 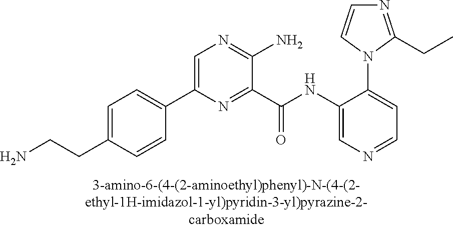 [SMILES: CCc1nccn1-c1ccncc1NC(=O)c1nc(-c2ccc(CCN)cc2)cnc1N] | ¹H NMR (300 MHz, Methanol-d₄) δ: 0.90- 1.17 (m, 3H), 2.60-2.62 (m, 2H), 2.90-2.92 (m, 2H), 3.04 (s, 2H), 7.27-7.40 (m, 4H), 7.61-7.63 (m, 3H), 8.55 (s, 1H), 8.75 (s, 1H), 9.88 (s, 1H). |
| 141 | 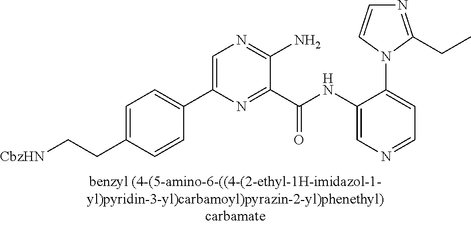 [SMILES: CCc1nccn1-c1ccncc1NC(=O)c1nc(-c2ccc(CCNC(=O)OCc3ccccc3)cc2)cnc1N] | ¹H NMR (300 MHz, Methanol-d₄) δ: 1.12- 1.17 (m, 3H), 2.55-2.62 (m, 2H), 2.85-2.89 (m, 2H), 3.40-3.47 (m, 2H), 5.07 (s, 2H), 7.24-7.37 (m, 9H), 7.51-7.60 (m, 3H), 8.53 (d, J = 5.1 Hz, 1H), 8.74 (s, 1H), 9.87 (s, 1H). |
| 142 | 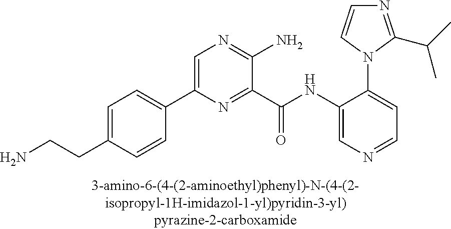 [SMILES: CC(C)c1nccn1-c1ccncc1NC(=O)c1nc(-c2ccc(CCN)cc2)cnc1N] | ¹H NMR (400 MHz, Methanol-d₄) δ: 1.28 (s, 6H), 2.78-2.92 (m, 3H), 3.01-3.05 (m, 2H), 7.27-7.33 (m, 2H), 7.35-7.44 (m, 2H), 7.55-7.56 (m, 1H), 7.62-7.66 (m, 2H), 8.55 (d, J = 5.2 Hz, 1H), 8.78 (s, 1H), 9.93 (s, 1H). |
| 143 | 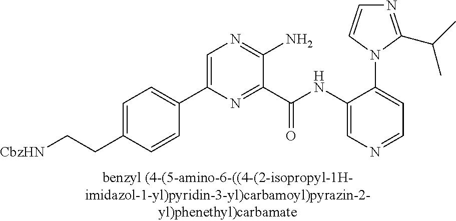 [SMILES: CC(C)c1nccn1-c1ccncc1NC(=O)c1nc(-c2ccc(CCNC(=O)OCc3ccccc3)cc2)cnc1N] | ¹H NMR (300 MHz, Methanol-d₄) δ: 1.02 (s, 6H), 2.77-2.92 (m, 3H), 3.43-3.48 (m, 2H), 5.10 (s, 2H), 7.26-7.39 (m, 9H), 7.53- 7.68 (m, 3H), 8.55 (d, J = 5.1 Hz, 1H), 8.76 (s, 1H), 9.92 (s, 1H). |
| 144 | 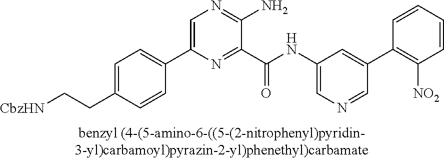 [SMILES: Nc1ncc(-c2ccc(CCNC(=O)OCc3ccccc3)cc2)nc1C(=O)Nc1cncc(-c2ccccc2[N+](=O)[O-])c1] | ¹H NMR (400 MHz, DMSO-d₆) δ: 2.78- 2.81 (m, 2H), 3.26-3.30 (m, 2H), 5.01 (s, 2H), 7.28-7.37 (m, 8H), 7.62-7.64 (m, 3H), 7.74-7.76 (m, 1H), 7.84-7.88 (m, 1H), 8.11- 8.14 (m, 3H), 8.29 (s, 1H), 8.34 (s, 1H), 8.92 (s, 1H), 9.11 (s, 1H), 10.67 (s, 1H). |
| 145 | 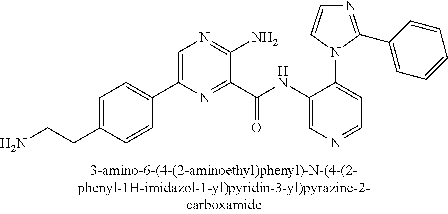 [SMILES: NCCc1ccc(-c2cnc(N)c(C(=O)Nc3cnccc3-n3ccnc3-c3ccccc3)n2)cc1] | ¹H NMR (400 MHz, Methanol-d₄) δ: 3.00- 3.08 (m, 2H), 3.28-3.33 (m, 2H), 7.31-7.35 (m, 2H), 7.38-7.53 (m, 5H), 7.59 (d, J = 5.2 Hz, 1H), 7.74 (t, J = 2.0 Hz, 1H), 7.83 (d, J = 1.2 Hz, 1H), 7.92 (d, J = 8.0 Hz, 2H), 8.59 (d, J = 5.2 Hz, 1H), 8.82 (s, 1H), 9.34 (s, 1H). |
| 146 | 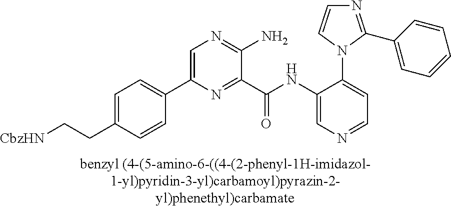 [SMILES: Nc1ncc(-c2ccc(CCNC(=O)OCc3ccccc3)cc2)nc1C(=O)Nc1cnccc1-n1ccnc1-c1ccccc1] | ¹H NMR (300 MHz, Methanol-d₄) δ: 2.87- 2.92 (m, 2H), 3.43-3.73 (m, 2H), 5.10 (s, 2H), 7.14-7.39 (m, 12H), 7.47 (s, 1H), 7.53- 5.57 (m, 2H), 7.67-7.70 (m, 2H), 8.50 (d, J = 5.4 Hz, 1H). 8.72 (s, 1H). 9.49 (s, 1H). |
| 147 | 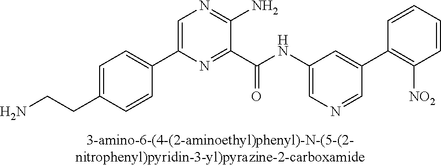 [SMILES: NCCc1ccc(-c2cnc(N)c(C(=O)Nc3cncc(-c4ccccc4[N+](=O)[O-])c3)n2)cc1] | ¹H NMR (400 MHz, DMSO-d₆) δ: 2.68- 2.82 (m, 4H), 3.13-3.32 (m, 2H), 7.32 (d, J = 8.0 Hz, 2H), 7.72-7.76 (m, 3H), 7.85- 7.88 (m, 1H), 7.93-7.96 (m, 1H), 8.12- 8.20 (m, 3H), 8.33-8.34 (m, 2H), 8.92 (s, 1H), 9.11 (s, 1H), 10.67 (s, 1H). |
| 148 | 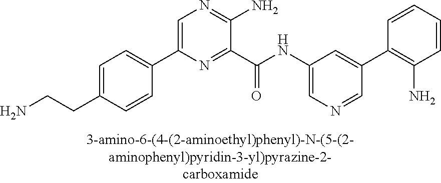 [SMILES: NCCc1ccc(-c2cnc(N)c(C(=O)Nc3cncc(-c4ccccc4N)c3)n2)cc1] | ¹H NMR (400 MHz, DMSO-d₆) δ: 2.62- 2.73 (m, 3H), 2.81-2.97 (m, 2H), 3.20-3.23 (m, 1H), 4.98 (s, 2H), 6.66-6.71 (m, 1H), 6.75-6.81 (m, 1H), 7.06-7.13 (m, 2H), 7.33 (d, J = 8.0 Hz, 2H), 7.65 (s, 2H), 8.14 (d, J = 7.8 Hz, 2H), 8.29 (s, 1H), 8.38 (s, 1H), 8.92 (s, 1H), 9.05 (s, 1H), 10.58 (s, 1H). |
| 149 | 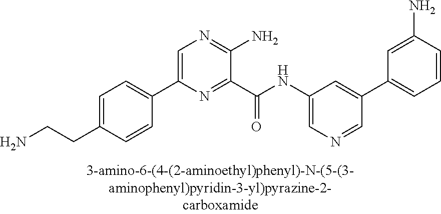 [SMILES: NCCc1ccc(-c2cnc(N)c(C(=O)Nc3cncc(-c4cccc(N)c4)c3)n2)cc1] | ¹H NMR (300 MHz, Methanol-d₄) δ: 3.01- 3.06 (m, 2H), 3.21-3.23 (m, 2H), 7.18-7.19 (m, 1H), 7.42-7.53 (m, 5H), 8.12 (d, J = 8.1 Hz, 2H), 8.68 (s, 1H), 8.01-8.83 (m, 2H), 9.20 (s, 1H). |
| 150 | 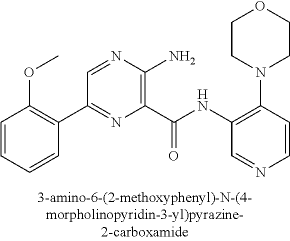 [SMILES: COc1ccccc1-c1cnc(N)c(C(=O)Nc2cnccc2N2CCOCC2)n1] | ¹H NMR (400 MHz, DMSO-d₆) δ: 2.92 (s, 4H), 3.69 (s, 4H), 3.88 (s, 3H), 7.12-7.21 (m, 3H), 7.34-7.64 (m, 3H), 7.87 (s, 1H), 8.30 (s, 1H), 8.81 (s, 1H), 9.42 (s, 1H), 10.43 (s, 1H). |
| 151 | 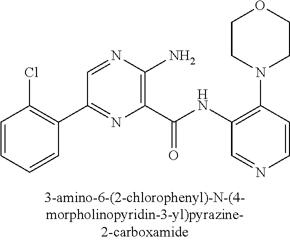 [SMILES: Nc1ncc(-c2ccccc2Cl)nc1C(=O)Nc1cnccc1N1CCOCC1] | ¹H NMR (400 MHz, DMSO-d₆) δ: 2.88 (s, 4H), 3.51 (s, 4H), 7.19 (d, J = 4.0 Hz, 1H), 7.51-7.83 (m, 6H), 8.29 (d, J = 4.0 Hz, 1H), 8.64 (s, 1H), 9.41 (s, 1H), 10.38 (s, 1H). |
| 152 | 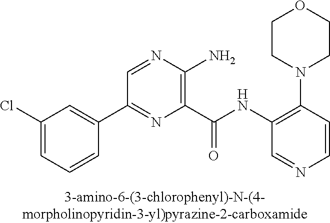 [SMILES: Nc1ncc(-c2cccc(Cl)c2)nc1C(=O)Nc1cnccc1N1CCOCC1] | ¹H NMR (400 MHz, DMSO-d₆) δ: 2.98 (s, 4H), 3.71 (s, 4H), 7.21 (s, 1H), 7.52-7.65 (m, 2H), 7.96 (s, 2H), 8.00-8.31 (m, 2H), 8.44 (d, J = 8.8 Hz, 1H), 9.05 (s, 1H), 9.35 (s, 1H), 10.38 (s, 1H). |
| 153 | 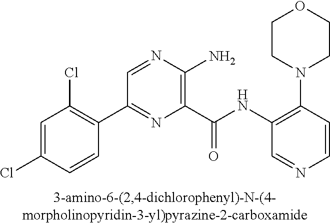 [SMILES: Nc1ncc(-c2ccc(Cl)cc2Cl)nc1C(=O)Nc1cnccc1N1CCOCC1] | ¹H NMR (300 MHz, DMSO-d₆) δ: 2.74- 2.96 (m, 4H), 3.52-3.66 (m, 4H), 7.18 (d, J = 5.4 Hz, 1H), 7.58-7.78 (m, 1H), 7.78- 8.03 (m, 4H), 8.29 (d, J = 5.4 Hz, 1H), 8.65 (s, 1H), 9.39 (s, 1H), 10.34 (s, 1H). |
| 154 | 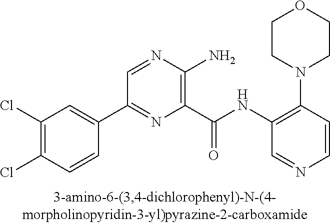 [SMILES: Nc1ncc(-c2ccc(Cl)c(Cl)c2)nc1C(=O)Nc1cnccc1N1CCOCC1] | ¹H NMR (300 MHz, DMSO-d₆) δ: 2.30 (s, 4H), 3.71 (s, 4H), 7.19 (d, J = 5.4 Hz, 1H), 7.75-8.23 (m, 4H), 8.31 (d, J = 5.4 Hz, 1H), 8.43 (s, 1H), 9.02 (s, 1H), 9.20 (s, 1H), 10.30 (s, 1H). |
| 155 | 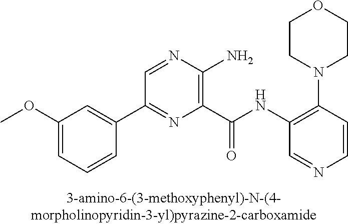 [SMILES: COc1cccc(-c2cnc(N)c(C(=O)Nc3cnccc3N3CCOCC3)n2)c1] | ¹H NMR (400 MHz, DMSO-d₆) δ: 2.96- 2.98 (m, 4H), 3.70-3.73 (m, 4H), 3.85 (s, 3H), 7.02-7.05 (m, 1H), 7.23 (d, J = 6.0 Hz, 1H), 7.44 (t, J = 8.0 Hz, 1H), 7.67-7.79 (m, 4H), 8.32 (d, J = 5.2 Hz, 1H), 8.98 (s, 1H), 9.32 (s, 1H), 10.41 (s, 1H). |
| 156 | 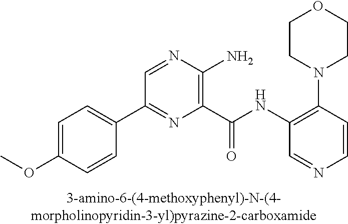 [SMILES: COc1ccc(-c2cnc(N)c(C(=O)Nc3cnccc3N3CCOCC3)n2)cc1] | ¹H NMR (300 MHz, DMSO-d₆) δ: 3.00 (s, 3H), 3.75-3.86 (m, 8H), 7.09-7.11 (m, 1H), 7.26 (s, 1H), 7.59-7.70 (m, 4H), 8.11-8.20 (m, 1H), 8.32 (s, 1H), 8.94 (s, 1H), 9.36 (s, 1H), 10.48 (s, 1H). |
| 157 | 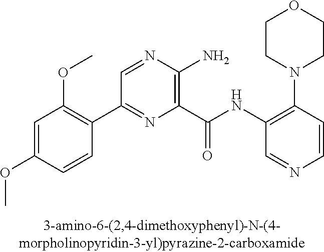 [SMILES: COc1ccc(-c2cnc(N)c(C(=O)Nc3cnccc3N3CCOCC3)n2)c(OC)c1] | ¹H NMR (300 MHz, DMSO-d₆) δ: 2.91- 2.94 (m, 4H), 3.62-3.64 (m, 4H), 3.85 (s, 3H), 3.89 (s, 3H), 6.71-6.75 (m, 2H), 7.22 (d, J = 5.4 Hz, 1H), 7.60 (s, 2H), 7.81 (d, J = 8.7 Hz, 1H), 8.30 (d, J = 5.4 Hz, 1H), 8.77 (s, 1H), 9.41 (s, 1H), 10.43 (s, 1H). |
| 158 | 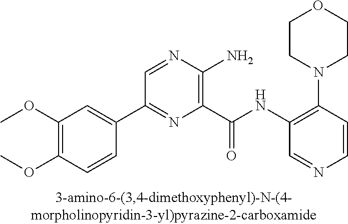 [SMILES: COc1ccc(-c2cnc(N)c(C(=O)Nc3cnccc3N3CCOCC3)n2)cc1OC] | ¹H NMR (400 MHz, DMSO-d₆) δ: 2.97- 3.00 (m, 4H), 3.67-3.69 (m, 4H), 3.83 (s, 3H), 3.88 (s, 3H), 7.08 (d, J = 8.8 Hz, 1H), 7.20 (d, J = 5.6 Hz, 1H), 7.64-7.71 (m, 4H), 8.31 (d, J = 5.6 Hz, 1H), 8.94 (s, 1H), 9.22 (s, 1H), 10.35 (s, 1H). |
| 159 | 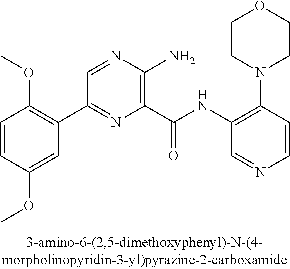 [SMILES: COc1ccc(OC)c(-c2cnc(N)c(C(=O)Nc3cnccc3N3CCOCC3)n2)c1] | ¹H NMR (300 MHz, DMSO-d₆) δ: 2.93- 2.94 (m, 4H), 3.62-3.65 (m, 4H), 3.85 (s, 3H), 3.89 (s, 3H), 6.68-6.75 (m, 2H), 7.22 (d, J = 5.1 Hz, 1H), 7.63 (s, 2H), 7.80-7.83 (m, 1H), 8.30 (d, J = 5.1 Hz, 1H), 8.77 (s, 1H), 9.38 (s, 1H), 10.35 (s, 1H). |
| 160 | 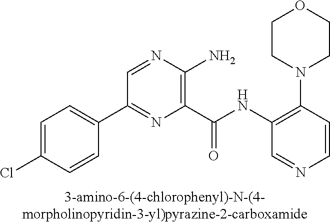 [SMILES: Nc1ncc(-c2ccc(Cl)cc2)nc1C(=O)Nc1cnccc1N1CCOCC1] | ¹H NMR (300 MHz, DMSO-d₆) δ: 3.06 (s, 4H), 3.70 (s, 4H), 7.21 (d, J = 4.8 Hz, 1H), 7.63-7.70 (m, 2H), 7.95 (s, 2H), 8.17-8.33 (m, 2H), 8.44 (d, J = 8.7 Hz, 1H), 8.99 (s, 1H), 9.33 (s, 1H), 10.50 (s, 1H). |
| 161 | 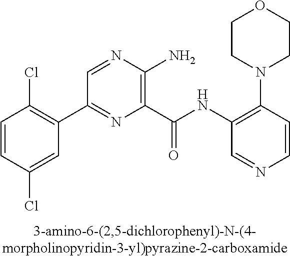 [SMILES: Nc1ncc(-c2cc(Cl)ccc2Cl)nc1C(=O)Nc1cnccc1N1CCOCC1] | ¹H NMR (400 MHz, Chloroform-d₁) δ: 2.30-3.03 (m, 4H), 3.67-3.74 (m, 4H), 7.04 (d, J = 5.2 Hz, 1H), 7.39-7.44 (m, 1H), 7.66 (d, J = 5.6 Hz, 1H), 7.72 (s, 1H), 8.39 (d, J = 4.0 Hz, 1H), 9.70 (s, 1H), 9.73 (s, 1H), 10.36 (s, 1H). (two active hydrogens were not found) |
| 162 | 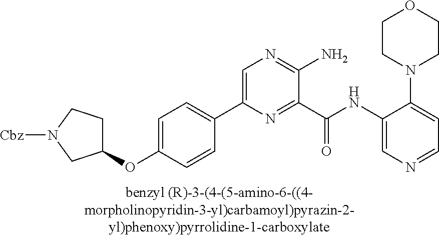 [SMILES: Nc1ncc(-c2ccc(O[C@@H]3CCN(C(=O)OCc4ccccc4)C3)cc2)nc1C(=O)Nc1cnccc1N1CCOCC1] | [M + H]⁺: 596.3 |
| 163 | 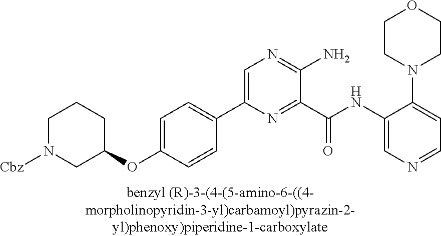 [SMILES: Nc1ncc(-c2ccc(O[C@@H]3CCCN(C(=O)OCc4ccccc4)C3)cc2)nc1C(=O)Nc1cnccc1N1CCOCC1] | [M + H]⁺: 610.3 |
| 164 | 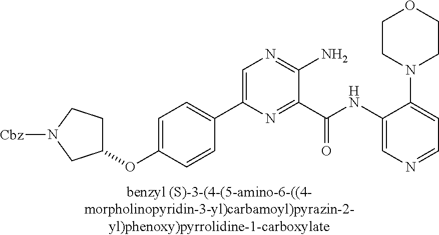 | [M + H]⁺: 596.3 |
| 165 | 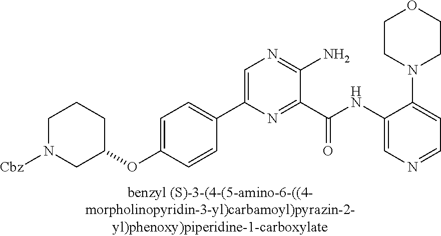 | [M + H]⁺: 610.3 |
| 166 | 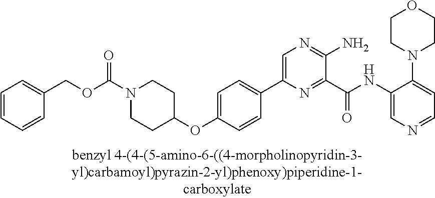 [SMILES: Nc1ncc(-c2ccc(OC3CCN(C(=O)OCc4ccccc4)CC3)cc2)nc1C(=O)Nc1cnccc1N1CCOCC1] | [M + H]⁺: 610.3 |
| 167 | 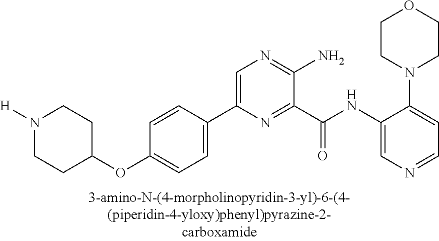 [SMILES: Nc1ncc(-c2ccc(OC3CCNCC3)cc2)nc1C(=O)Nc1cnccc1N1CCOCC1] | [M + H]⁺: 476.3 |
| 168 | 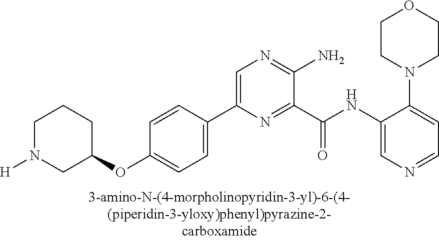 [SMILES: Nc1ncc(-c2ccc(O[C@@H]3CCCNC3)cc2)nc1C(=O)Nc1cnccc1N1CCOCC1] | [M + H]⁺: 476.3 |
| 169 | 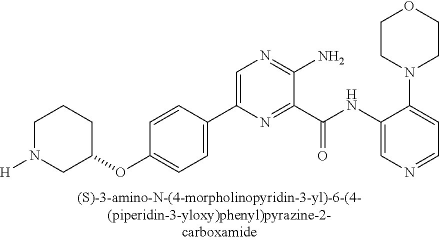 | [M + H]⁺: 476.3 |
| 170 | 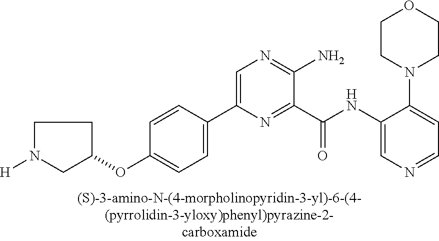 | [M + H]⁺: 596.3 |
| 171 | 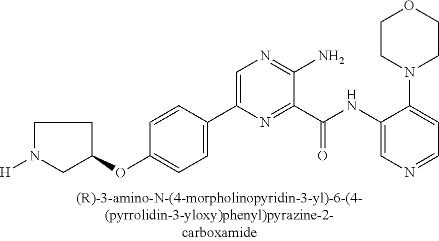 [SMILES: Nc1ncc(-c2ccc(O[C@@H]3CCNC3)cc2)nc1C(=O)Nc1cnccc1N1CCOCC1] | [M + H]⁺: 596.3 |
| 172 | 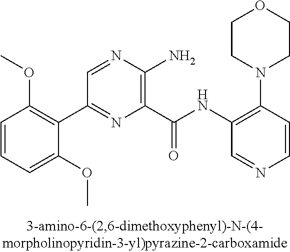 [SMILES: COc1cccc(OC)c1-c1cnc(N)c(C(=O)Nc2cnccc2N2CCOCC2)n1] | ¹H NMR (400 MHz, DMSO-d₆) δ: 2.85 (s, 4H), 3.52 (s, 4H), 3.70 (s, 6H), 6.81 (d, J = 8.8 Hz, 2H), 7.16 (s, 1H), 7.42 (t, J = 8.4 Hz, 1H), 7.63 (s, 2H), 8.24-8.27 (m, 2H), 9.41 (s, 1H), 10.33 (s, 1H). |
| 173 | 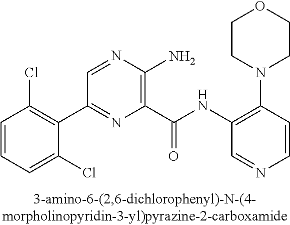 [SMILES: Nc1ncc(-c2c(Cl)cccc2Cl)nc1C(=O)Nc1cnccc1N1CCOCC1] | ¹H NMR (300 MHz, DMSO-d₆) δ: 2.73- 2.87 (m, 4H), 3.34-3.43 (m, 4H), 7.17 (d, J = 5.0 Hz, 1H), 7.54-7.59 (m, 1H), 7.67- 7.77 (m, 2H), 7.79 (s, 2H), 7.90 (s, 1H), 8.48 (s, 1H), 9.45 (s, 1H), 10.30 (s, 1H). |
| 174 | 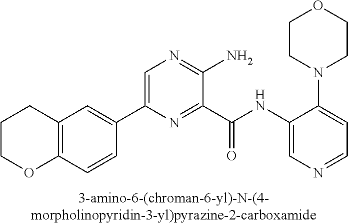 [SMILES: Nc1ncc(-c2ccc3c(c2)CCCO3)nc1C(=O)Nc1cnccc1N1CCOCC1] | ¹H NMR (300 MHz, DMSO-d₆) δ: 1.98- 2.00 (m, 2H), 2.83-2.88 (m, 2H), 2.97 (s, 4H), 3.73 (s, 4H), 4.19-4.22 (m, 2H), 6.85 (d, J = 8.4 Hz, 1H), 7.22 (d, J = 5.1 Hz, 1H), 7.66 (s, 2H), 7.84-7.87 (m, 2H), 8.31 (d, J = 5.1 Hz, 1H), 8.87 (s, 1H), 9.34 (s, 1H), 10.36 (s, 1H). |
| 175 | 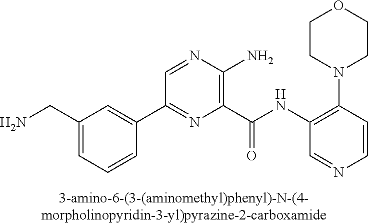 [SMILES: NCc1cccc(-c2cnc(N)c(C(=O)Nc3cnccc3N3CCOCC3)n2)c1] | ¹H NMR (400 MHz, DMSO-d₆) δ: 2.96- 2.98 (m, 4H), 3.67-3.69 (m, 4H), 4.13 (s, 2H), 7.22 (d, J = 5.2 Hz, 1H), 7.52-7.60 (m, 2H), 7.83 (s, 2H), 8.18-8.32 (m, 5H), 8.94 (s, 1H), 9.26 (s, 1H), 10.39 (s, 1H). |
| 176 | 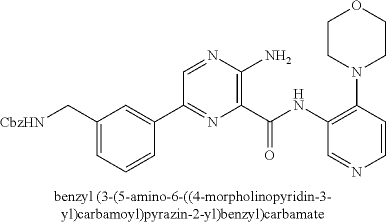 [SMILES: Nc1ncc(-c2cccc(CNC(=O)OCc3ccccc3)c2)nc1C(=O)Nc1cnccc1N1CCOCC1] | ¹H NMR (400 MHz, DMSO-d₆) δ: 2.96 (s, 4H), 3.69 (s, 4H), 4.33 (d, J = 6.0 Hz, 2H), 5.05 (s, 2H), 7.21-7.22 (m, 1H), 7.30-7.35 (m, 5H), 7.36-7.50 (m, 2H), 7.53-7.57 (s, 2H), 7.78 (s, 1H), 7.88-7.91 (m, 1H), 8.03 (d, J = 8.0 Hz, 1H), 8.32 (d, J = 5.2 Hz, 1H), 8.91 (s, 1H), 9.30 (s, 1H), 10.38 (s, 1H). |
| 177 | 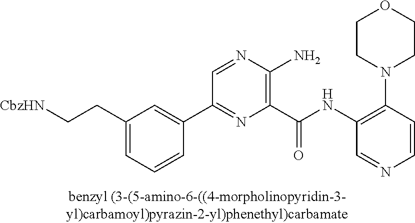 [SMILES: Nc1ncc(-c2cccc(CCNC(=O)OCc3ccccc3)c2)nc1C(=O)Nc1cnccc1N1CCOCC1] | ¹H NMR (400 MHz, DMSO-d₆) δ: 2.83- 2.86 (m, 2H), 2.97 (s, 4H), 3.30-3.35 (m, 2H), 3.69 (s, 4H), 4.99 (s, 2H), 7.22-7.23 (m, 1H), 7.28-7.35 (m, 6H), 7.39-7.46 (m, 2H), 7.77 (s, 2H), 7.94 (s, 1H), 8.00 (d, J = 8.0 Hz, 1H), 8.32 (d, J = 9.2 Hz, 1H), 8.95 (s, 1H), 9.31 (s, 1H), 10.37 (s, 1H). |
| 178 | 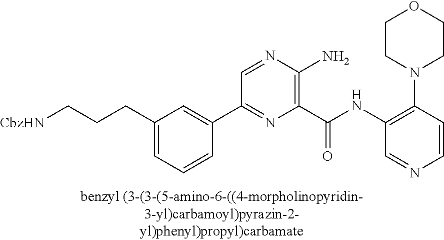 [SMILES: Nc1ncc(-c2cccc(CCCNC(=O)OCc3ccccc3)c2)nc1C(=O)Nc1cnccc1N1CCOCC1] | ¹H NMR (300 MHz, DMSO-d₆) δ: 1.76- 1.78 (m, 2H), 2.64-2.77 (m, 2H), 2.93-3.05 (m, 6H), 3.67 (s, 4H), 4.95 (s, 2H), 7.13- 7.19 (m, 1H), 7.22-7.40 (m, 8H), 7.60-7.90 (m, 2H), 7.91-7.95 (m, 2H), 8.28 (d, J = 5.7 Hz, 1H), 8.92 (s, 1H), 9.29 (s, 1H), 10.33 (s, 1H). |
| 179 | 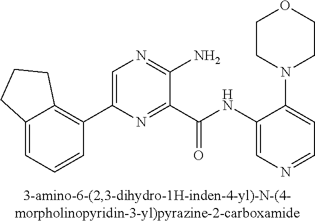 [SMILES: Nc1ncc(-c2cccc3c2CCC3)nc1C(=O)Nc1cnccc1N1CCOCC1] | ¹H NMR (300 MHz, Chloroform-d₁) δ: 1.97-2.22 (m, 2H), 2.97-3.04 (m, 6H), 3.16-3.21 (m, 2H), 3.69 (s, 4H), 7.00 (d, J = 6.0 Hz, 1H), 7.20-7.46 (m, 2H), 7.55- 7.70 (m, 1H), 8.37 (d, J = 6.0 Hz, 1H), 8.55 (s, 1H), 9.61 (s, 1H), 10.48 (s, 1H). (two active hydrogens were not found) |
| 180 | 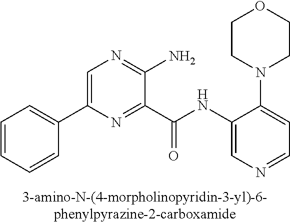 [SMILES: Nc1ncc(-c2ccccc2)nc1C(=O)Nc1cnccc1N1CCOCC1] | ¹H NMR (400 MHz, Methanol-d₄) δ: 3.01- 3.08 (m, 4H), 3.82-3.83 (m, 4H), 7.28 (d, J = 5.2 Hz, 1H), 7.44-7.54 (m, 3H), 8.08 (d, J = 7.2 Hz, 2H), 8.29 (d, J = 5.2 Hz, 1H), 8.81 (s, 1H), 9.51 (s, 1H). |
| 181 | 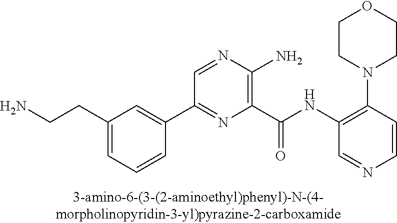 [SMILES: NCCc1cccc(-c2cnc(N)c(C(=O)Nc3cnccc3N3CCOCC3)n2)c1] | ¹H NMR (400 MHz, DMSO-d₆) δ: 2.83 (s, 6H), 3.11-3.69 (m, 7H), 7.22 (d, J = 4.8 Hz, 1H), 7.33-7.34 (m, 1H), 7.46-7.50 (m, 1H), 7.79 (s, 2H), 7.99-8.05 (m, 2H), 8.59-8.63 (s, 2H), 8.97 (s, 1H), 9.48 (s, 1H), 10.55 (s, 1H). |
| 182 | 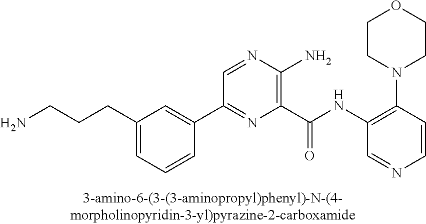 [SMILES: NCCCc1cccc(-c2cnc(N)c(C(=O)Nc3cnccc3N3CCOCC3)n2)c1] | ¹H NMR (300 MHz, Methanol-d₄) δ: 2.07- 2.11 (m, 2H), 2.73-2.87 (m, 2H), 2.93-3.10 (m, 2H), 3.54-3.57 (m, 4H), 3.69-3.85 (m, 4H), 7.34 (d, J = 8.1 Hz, 1H), 7.41-7.58 (m, 2H), 7.90-7.96 (m, 2H), 8.34 (d, J = 6.9 Hz, 1H), 8.82 (s, 1H), 9.13 (s, 1H). |
| 183 | 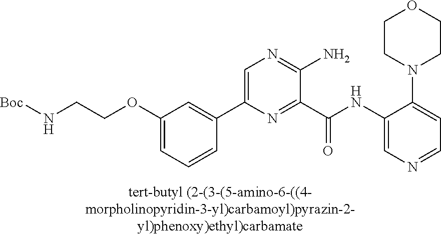 [SMILES: CC(C)(C)OC(=O)NCCOc1cccc(-c2cnc(N)c(C(=O)Nc3cnccc3N3CCOCC3)n2)c1] | ¹H NMR (400 MHz, Chloroform-d₁) δ: 1.47 (s, 9H), 3.03-3.04 (m, 4H), 3.59-3.60 (m, 2H), 3.94-3.96 (m, 4H), 4.12-4.14 (m, 2H), 5.54 (s, 1H), 6.90 (d, J = 5.2 Hz, 1H), 7.10 (d, J = 6.0 Hz, 1H), 7.45 (d, J = 6.0 Hz, 1H), 7.53 (d, J = 8.0 Hz, 1H), 7.60 (d, J = 8.0 Hz, 1H), 8.40 (d, J = 5.2 Hz, 1H), 8.74 (s, 1H), 9.73 (s, 1H), 10.47 (s, 1H). (two active hydrogens were not found) |
| 184 | 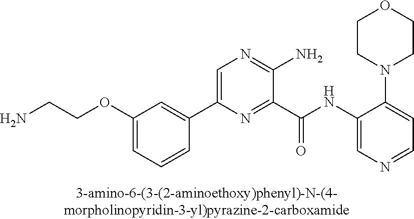 [SMILES: NCCOc1cccc(-c2cnc(N)c(C(=O)Nc3cnccc3N3CCOCC3)n2)c1] | ¹H NMR (400 MHz, Methanol-d₄) δ: 2.91- 3.10 (m, 4H), 3.21-3.41 (m, 2H), 3.81-3.92 (m, 4H), 4.33-4.41 (m, 2H), 7.24 (s, 1H), 7.41-7.55 (m, 1H), 7.65 (s, 1H), 7.73 (d, J = 5.2 Hz, 2H), 8.37 (s, 1H), 8.67 (s, 1H), 9.78 (s, 1H). |
| 185 | 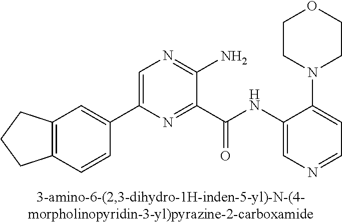 [SMILES: Nc1ncc(-c2ccc3c(c2)CCC3)nc1C(=O)Nc1cnccc1N1CCOCC1] | ¹H NMR (300 MHz, DMSO-d₆) δ: 2.05- 2.10 (m, 2H), 2.90-2.98 (m, 8H), 3.74-3.76 (m, 4H), 7.25 (d, J = 5.4 Hz, 1H), 7.36 (d, J = 7.8 Hz, 1H), 7.72 (s, 2H), 7.90-7.93 (m, 1H), 8.03 (s, 1H), 8.31 (d, J = 5.4 Hz, 1H), 8.94 (s, 1H), 9.39 (s, 1H), 10.42 (s, 1H). |
| 186 | 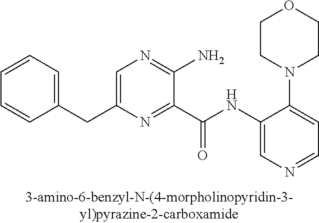 [SMILES: Nc1ncc(Cc2ccccc2)nc1C(=O)Nc1cnccc1N1CCOCC1] | ¹H NMR (400 MHz, Methanol-d₄) δ: 2.98- 3.05 (m, 4H), 3.74-3.81 (m, 4H), 4.16 (s, 2H), 7.18-7.37 (m, 6H), 8.17-8.18 (m, 1H), 8.18-8.28 (m, 1H), 9.44 (s, 1H). |
| 187 | 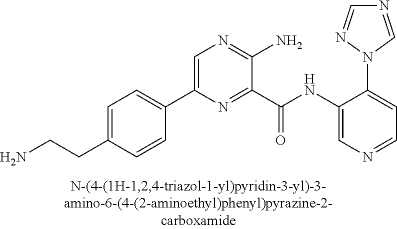 [SMILES: NCCc1ccc(-c2cnc(N)c(C(=O)Nc3cnccc3-n3cncn3)n2)cc1] | ¹H NMR (400 MHz, Methanol-d₄) δ: 3.02- 3.15 (m, 2H), 3.28-3.31 (m, 2H), 7.51 (d, J = 8.4 Hz, 2H), 7.92 (d, J = 5.6 Hz, 1H), 8.08-8.15 (m, 2H), 8.36 (s, 1H), 8.53 (d, J = 5.6 Hz, 1H), 8.81 (s, 1H), 9.33 (s, 1H), 10.00 (s, 1H). |
| 188 | 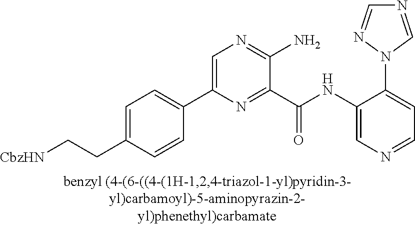 [SMILES: Nc1ncc(-c2ccc(CCNC(=O)OCc3ccccc3)cc2)nc1C(=O)Nc1cnccc1-n1cncn1] | ¹H NMR (400 MHz, Chloroform-d₁) δ: 1.27 (s, 2H), 2.95 (s, 2H), 3.48-3.56 (m, 2H), 4.86-4.89 (s, 1H), 5.14 (s, 2H), 7.15-7.43 (m, 8H), 7.99 (d, J = 12 Hz, 2H), 8.27 (s, 1H), 8.57 (s, 1H), 8.66 (s, 1H), 8.71-8.76 (m, 1H), 10.05 (s, 1H), 12.08 (s, 1H). |
| 189 | 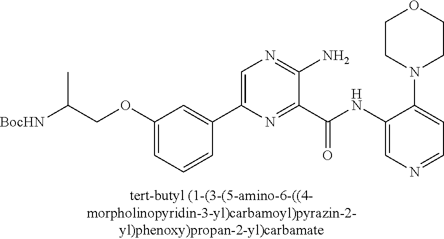 [SMILES: CC(COc1cccc(-c2cnc(N)c(C(=O)Nc3cnccc3N3CCOCC3)n2)c1)NC(=O)OC(C)(C)C] | ¹H NMR (400 MHz, Chloroform-d₁) δ: 1.32 (s, 3H), 1.44 (s, 9H), 3.11-3.13 (m, 4H), 3.89-3.90 (m, 4H), 4.03 (s, 2H), 4.10 (s, 1H), 5.01 (s, 1H), 7.50-7.54 (m, 1H), 7.15 (d, J = 5.2 Hz, 1H), 7.44 (t, J = 8.0 Hz, 1H), 7.50-7.54 (m, 2H), 8.40 (s, 1H), 8.73 (s, 1H), 9.68 (s, 1H), 10.37 (s, 1H). (two active hydrogens were not found) |
| 190 | 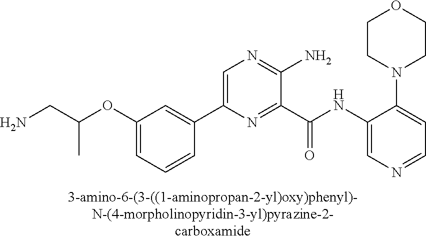 [SMILES: CC(CN)Oc1cccc(-c2cnc(N)c(C(=O)Nc3cnccc3N3CCOCC3)n2)c1] | ¹H NMR (400 MHz, Methanol-d₄) δ: 1.46 (d, J = 6.8 Hz, 3H), 3.20-3.23 (m, 1H), 3.31-3.35 (m, 2H), 3.51-3.59 (m, 4H), 3.79-3.86 (m, 4H), 7.12 (d, J = 6.8 Hz, 1H), 7.48-7.54 (m, 2H), 7.67 (s, 1H), 7.74 (d, J = 8.0 Hz, 1H), 8.36 (d, J = 6.8 Hz, 1H), 8.83 (s, 1H), 9.14 (s, 1H). |
| 191 | 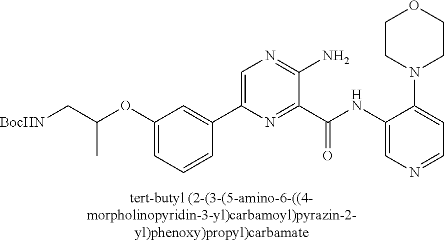 [SMILES: CC(CNC(=O)OC(C)(C)C)Oc1cccc(-c2cnc(N)c(C(=O)Nc3cnccc3N3CCOCC3)n2)c1] | ¹H NMR (400 MHz, Chloroform-d₁) δ: 1.34 (s, 3H), 1.45 (s, 9H), 3.04-3.06 (m, 4H), 3.30-3.31 (m, 1H), 3.52-3.53 (m, 1H), 3.94-3.96 (m, 4H), 4.65 (s, 1H), 5.39 (s, 1H), 7.00 (d, J = 9.2 Hz, 1H), 7.10 (d, J = 1.6 Hz, 1H), 7.44 (t, J = 8.0 Hz, 1H), 7.52 (d, J = 7.6 Hz, 1H), 7.62 (s, 1H), 8.40 (s, 1H), 8.74 (s, 1H), 9.73 (s, 1H), 10.46 (s, 1H). (two active hydrogens were not found) |
| 192 | 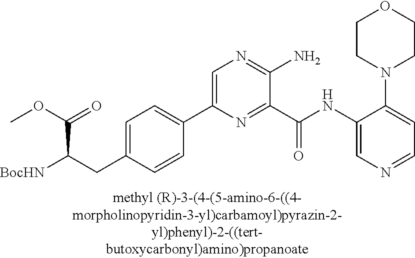 [SMILES: COC(=O)[C@@H](Cc1ccc(-c2cnc(N)c(C(=O)Nc3cnccc3N3CCOCC3)n2)cc1)NC(=O)OC(C)(C)C] | ¹H NMR (300 MHz, Methanol-d₄) δ: 1.33 (s, 9H), 2.97-3.04 (m, 5H), 3.32 (s, 1H), 3.73-3.79 (m, 7H), 4.11-4.46 (m, 1H), 7.22- 7.33 (m, 3H), 7.49-7.61 (m, 1H), 7.92 (d, J = 7.2 Hz, 2H), 8.25-8.26 (m, 1H), 8.66 (s, 1H), 9.44 (s, 1H). (one active hydrogen was not exchanged with Methanol-d₄) |
| 193 | 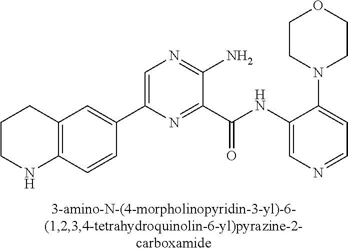 [SMILES: Nc1ncc(-c2ccc3c(c2)CCCN3)nc1C(=O)Nc1cnccc1N1CCOCC1] | ¹H NMR (400 MHz, DMSO-d₆) δ: 1.83- 1.84 (m, 2H), 2.75-2.78 (m, 2H), 2.95-2.97 (m, 4H), 3.24-3.32 (m, 2H), 3.75-3.77 (m, 4H), 6.06 (s, 1H), 6.54 (d, J = 7.6 Hz, 1H), 7.22 (d, J = 5.2 Hz, 1H), 7.49 (s, 2H), 7.62 (s, 1H), 7.68 (d, J = 8.4 Hz, 1H), 8.30 (d, J = 5.2 Hz, 1H), 8.79 (s, 1H), 9.37 (s, 1H), 10.38 (s, 1H). |
| 194 | 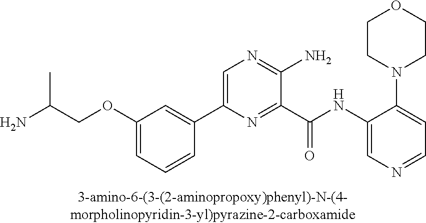 [SMILES: CC(N)COc1cccc(-c2cnc(N)c(C(=O)Nc3cnccc3N3CCOCC3)n2)c1] | ¹H NMR (400 MHz, Methanol-d₄) δ: 1.47 (d, J = 6.8 Hz, 3H), 3.32-3.33 (m, 4H), 3.52-3.88 (m, 5H), 4.11-4.16 (m, 1H), 4.31-4.35 (m, 1H), 7.12 (d, J = 6.8 Hz, 1H), 7.48-7.52 (m, 2H), 7.65 (s, 1H), 7.75 (d, J = 8.0 Hz, 1H), 8.35 (d, J = 6.8 Hz, 1H), 8.86 (s, 1H), 9.14 (s, 1H). |
| 195 | 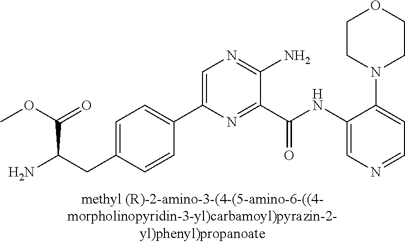 [SMILES: COC(=O)[C@H](N)Cc1ccc(-c2cnc(N)c(C(=O)Nc3cnccc3N3CCOCC3)n2)cc1] | ¹H NMR (400 MHz, DMSO-d₆) δ: 3.11- 3.20 (m, 2H), 3.52 (s, 4H), 3.68-3.71 (m, 7H), 4.40 (s, 1H), 7.35-7.42 (m, 3H), 7.71 (s, 2H), 8.21 (d, J = 8.0 Hz, 2H), 8.39 (d, J = 6.8 Hz, 1H), 8.50 (s, 2H), 8.81 (s, 1H), 9.00 (s, 1H), 10.48 (s, 1H). |
| 196 | 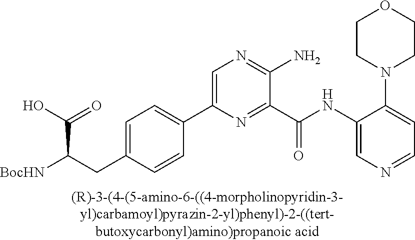 [SMILES: CC(C)(C)OC(=O)N[C@H](Cc1ccc(-c2cnc(N)c(C(=O)Nc3cnccc3N3CCOCC3)n2)cc1)C(=O)O] | ¹H NMR (400 MHz, Methanol-d₄) δ: 1.33- 1.37 (m, 3H), 1.40 (s, 6H), 3.05 (s, 5H), 3.20-3.21 (m, 1H), 3.85 (s, 4H), 4.31 (s, 1H), 7.28 (d, J = 5.2 Hz, 1H), 7.28-7.42 (m, 2H), 7.92-7.98 (m, 2H), 8.29 (d, J = 5.2 Hz, 1H), 8.77 (s, 1H), 9.47 (s, 1H). |
| 197 | 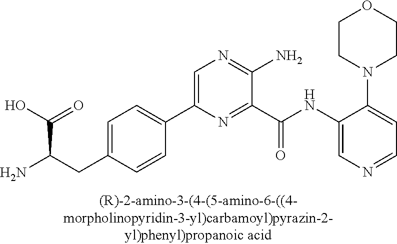 [SMILES: Nc1ncc(-c2ccc(C[C@@H](N)C(=O)O)cc2)nc1C(=O)Nc1cnccc1N1CCOCC1] | ¹H NMR (400 MHz, Methanol-d₄) δ: 3.26- 3.31 (m, 1H), 3.35-3.40 (m, 1H), 3.55-3.58 (m, 4H), 3.80-3.87 (m, 4H), 4.26-4.35 (m, 1H), 7.47-7.52 (m, 3H), 8.09 (d, J = 8.0 Hz, 2H), 8.36 (d, J = 6.8 Hz, 1H), 8.84 (s, 1H), 9.06 (s, 1H). |
| 198 | 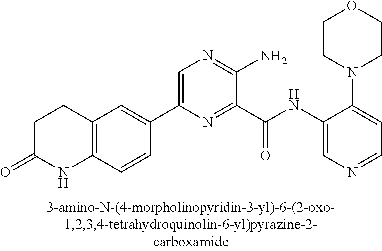 [SMILES: Nc1ncc(-c2ccc3c(c2)CCC(=O)N3)nc1C(=O)Nc1cnccc1N1CCOCC1] | ¹H NMR (400 MHz, DMSO-d₆ + D₂O) δ: 2.50-2.52 (m, 2H), 2.97-3.00 (m, 2H), 3.60 (s, 4H), 3.69-3.71 (m, 4H), 6.98 (d, J = 8.0 Hz, 1H), 7.43 (d, J = 6.8 Hz, 1H), 7.99-8.01 (m, 1H), 8.04 (s, 1H), 8.38 (d, J = 6.9 Hz, 1H), 8.75 (s, 1H), 8.92 (s, 1H). |
| 199 | 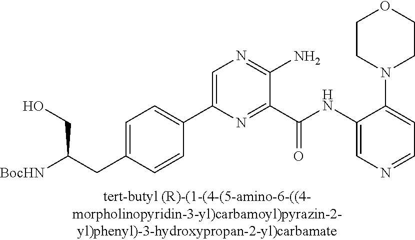 [SMILES: CC(C)(C)OC(=O)N[C@@H](CO)Cc1ccc(-c2cnc(N)c(C(=O)Nc3cnccc3N3CCOCC3)n2)cc1] | ¹H NMR (400 MHz, Methanol-d₄) δ: 1.34 (s, 9H), 2.76-2.78 (m, 1H), 2.96-3.07 (m, 5H), 3.62 (s, 2H), 3.82-3.96 (m, 5H), 7.28 (d, J = 5.6 Hz, 1H), 7.43 (d, J = 8.0 Hz, 2H), 8.01 (d, J = 8.0 Hz, 2H), 8.29 (d, J = 5.6 Hz, 1H), 8.78 (s, 1H), 9.49 (s, 1H). |
| 200 | 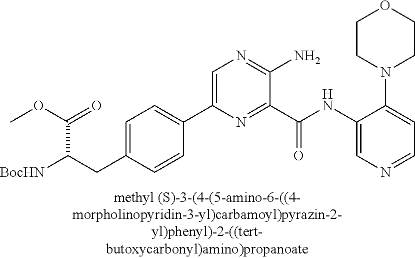 | ¹H NMR (400 MHz, Methanol-d₄) δ: 1.41 (m, 9H), 2.99-3.04 (m, 1H), 3.17-3.22 (m, 1H), 3.52-3.54 (m, 4H), 3.73 (s, 3H), 3.81- 3.84 (m, 4H), 4.43-4.46 (m, 1H), 7.39 (d, J = 8.0 Hz, 2H), 7.50 (d, J = 6.8 Hz, 1H), 8.02 (d, J = 8.0 Hz, 2H), 7.35 (d, J = 6.8 Hz, 1H), 8.83 (s, 1H), 9.11 (s, 1H). |
| 201 | 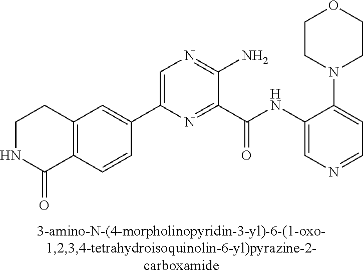 [SMILES: Nc1ncc(-c2ccc3c(c2)CCNC3=O)nc1C(=O)Nc1cnccc1N1CCOCC1] | ¹H NMR (400 MHz, DMSO-d₆) δ: 2.99- 3.02 (m, 2H), 3.43 (s, 6H), 3.77 (s, 4H), 7.37 (d, J = 6.0 Hz, 1H), 7.82 (s, 2H), 7.94 (d, J = 8.0 Hz, 1H), 7.99 (s, 1H), 8.13 (s, 1H), 8.19 (d, J = 7.6 Hz, 1H), 8.38 (s, 1H), 8.90 (s, 1H), 9.04 (s, 1H), 10.46 (s, 1H). |
| 202 | 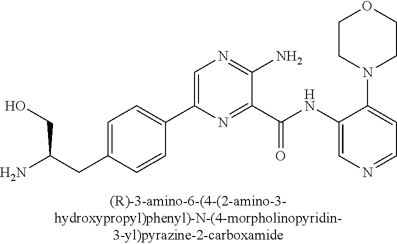 [SMILES: Nc1ncc(-c2ccc(C[C@@H](N)CO)cc2)nc1C(=O)Nc1cnccc1N1CCOCC1] | ¹H NMR (400 MHz, Methanol-d₄) δ: 2.67- 2.75 (m, 1H), 2.83-2.93 (m, 1H), 3.03 (s, 4H), 3.16-3.18 (m, 1H), 3.43-3.48 (m, 1H), 3.58-3.62 (m, 1H), 3.81 (s, 4H), 7.24 (d, J = 2.0 Hz, 1H), 7.43 (d, J = 8.0 Hz, 2H), 8.03 (d, J = 8.0 Hz, 2H), 8.29 (d, J = 8.0 Hz, 1H), 8.78 (s, 1H), 9.50 (s, 1H). |
| 203 | 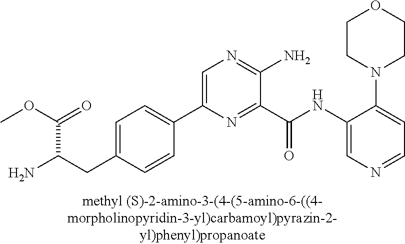 [SMILES: COC(=O)[C@@H](N)Cc1ccc(-c2cnc(N)c(C(=O)Nc3cnccc3N3CCOCC3)n2)cc1] | ¹H NMR (400 MHz, DMSO-d₆ + D₂O) δ: 3.10-3.21 (m, 2H), 3.50-3.51 (m, 4H), 3.68- 3.72 (m, 7H), 4.30-4.33 (m, 1H), 7.33-7.39 (m, 3H), 8.16 (d, J = 8.4 Hz, 2H), 8.33 (d, J = 6.8 Hz, 1H), 8.75 (s, 1H), 8.95 (s, 1H). |
| 204 | 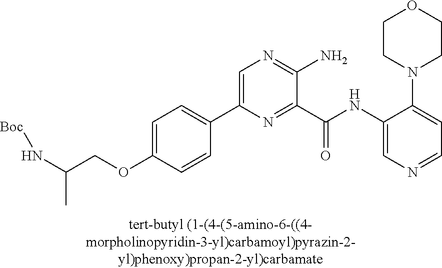 [SMILES: CC(COc1ccc(-c2cnc(N)c(C(=O)Nc3cnccc3N3CCOCC3)n2)cc1)NC(=O)OC(C)(C)C] | ¹H NMR (400 MHz, Chloroform-d₁) δ: 1.35 (s, 3H), 1.49 (s, 9H), 3.03-3.05 (m, 4H), 3.86-3.88 (m, 4H), 4.03 (d, J = 4.0 Hz, 2H), 4.12 (s, 1H), 4.82 (s, 1H), 7.05- 7.08 (m, 3H), 7.93-7.95 (m, 2H), 8.40 (d, J = 4.8 Hz, 1H), 8.69 (s, 1H), 9.65 (s, 1H), 10.47 (s, 1H). (two active hydrogens were not found) |
| 205 | 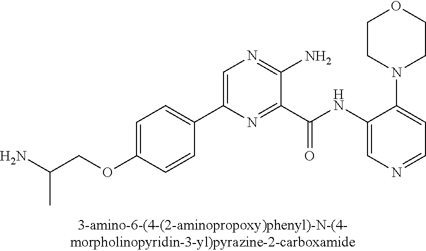 [SMILES: CC(N)COc1ccc(-c2cnc(N)c(C(=O)Nc3cnccc3N3CCOCC3)n2)cc1] | ¹H NMR (400 MHz, Methanol-d₄) δ: 1.47 (s, 3H), 3.31-3.33 (m, 4H), 3.66-3.83 (m, 5H), 4.08-4.12 (m, 1H), 4.28-4.32 (m, 1H), 7.18 (d, J = 8.8 Hz, 2H), 7.51 (d, J = 6.8 Hz, 1H), 8.05 (d, J = 8.8 Hz, 2H), 8.35 (d, J = 6.8 Hz, 1H), 8.80 (s, 1H), 9.11 (s, 1H). |
| 206 | 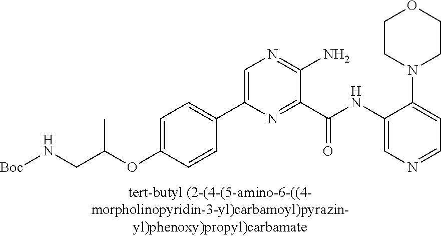 [SMILES: CC(CNC(=O)OC(C)(C)C)Oc1ccc(-c2cnc(N)c(C(=O)Nc3cnccc3N3CCOCC3)n2)cc1] | ¹H NMR (400 MHz, Chloroform-d₁) δ: 1.36 (s, 3H), 1.47 (s, 9H), 3.03-3.06 (m, 4H), 3.33-3.49 (m, 2H), 3.85-3.88 (m, 4H), 4.61 (s, 1H), 4.99 (s, 1H), 7.05-7.10 (m, 3H), 7.93 (d, J = 8.4 Hz, 2H), 8.40 (d, J = 5.2 Hz, 1H), 8.70 (s, 1H), 9.65 (s, 1H), 10.46 (s, 1H). (two active hydrogens were not found) |
| 207 | 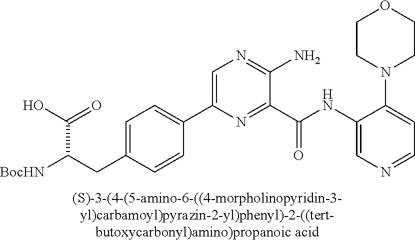 [SMILES: CC(C)(C)OC(=O)N[C@@H](Cc1ccc(-c2cnc(N)c(C(=O)Nc3cnccc3N3CCOCC3)n2)cc1)C(=O)O] | ¹H NMR (400 MHz, Methanol-d₄) δ: 1.45 (s, 9H), 3.01-3.09 (m, 5H), 3.23-3.24 (m, 1H), 3.83-3.85 (m, 4H), 4.40-4.41 (m, 1H), 7.29 (d, J = 5.6 Hz, 1H), 7.41 (d, J = 8.0 Hz, 2H), 7.98 (d, J = 8.0 Hz, 2H), 8.29 (d, J = 5.6 Hz, 1H), 8.77 (s, 1H), 9.45 (s, 1H). |
| 208 | 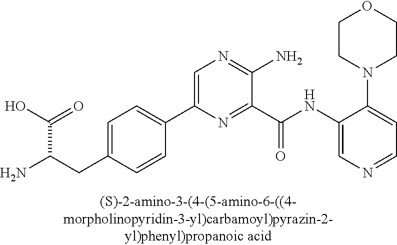 [SMILES: Nc1ncc(-c2ccc(C[C@H](N)C(=O)O)cc2)nc1C(=O)Nc1cnccc1N1CCOCC1] | ¹H NMR (400 MHz, Methanol-d₄) δ: 3.32- 3.38 (m, 1H), 3.40-3.49 (m, 1H), 3.69-3.75 (m, 4H), 3.81-3.90 (m, 4H), 4.30-4.33 (m, 1H), 7.47-7.52 (m, 3H), 8.10 (d, J = 8.0 Hz, 2H), 8.36 (d, J = 6.4 Hz, 1H), 8.85 (s, 1H), 9.06 (s, 1H). |
| 209 | 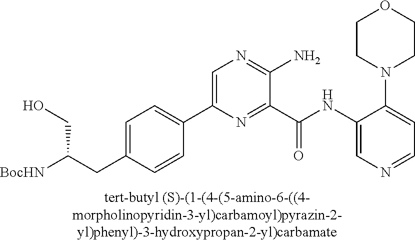 [SMILES: CC(C)(C)OC(=O)N[C@H](CO)Cc1ccc(-c2cnc(N)c(C(=O)Nc3cnccc3N3CCOCC3)n2)cc1] | ¹H NMR (400 MHz, Methanol-d₄) δ: 1.40 (s, 9H), 2.79-2.94 (m, 1H), 2.96-3.01 (m, 5H), 3.53-3.54 (m, 2H), 3.80-3.82 (m, 5H), 7.24 (d, J = 5.6 Hz, 1H), 7.39 (d, J = 8.0 Hz, 2H), 7.95-7.97 (d, J = 8.0 Hz, 2H), 8.26 (d, J = 5.2 Hz, 1H), 8.71 (s, 1H), 9.46 (s, 1H). |
| 210 | 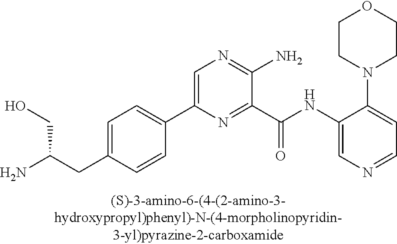 [SMILES: Nc1ncc(-c2ccc(C[C@H](N)CO)cc2)nc1C(=O)Nc1cnccc1N1CCOCC1] | ¹H NMR (400 MHz, Methanol-d₄) δ: 3.01- 3.05 (m, 2H), 3.52-3.60 (m, 6H), 3.72-3.75 (m, 1H), 3.80-3.82 (m, 4H), 7.46-7.53 (m, 3H), 8.08 (d, J = 8.0 Hz, 2H), 8.36 (d, J = 6.8 Hz, 1H), 8.85 (s, 1H), 9.10 (s, 1H). |
| 211 | 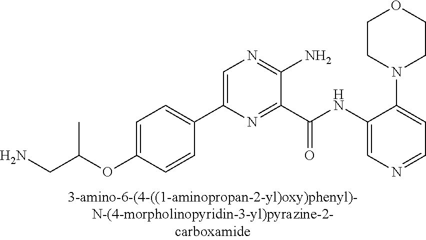 [SMILES: CC(CN)Oc1ccc(-c2cnc(N)c(C(=O)Nc3cnccc3N3CCOCC3)n2)cc1] | ¹H NMR (400 MHz, Methanol-d₄) δ: 1.47 (s, 3H), 3.20-3.33 (m, 2H), 3.53-3.55 (m, 4H), 3.81-3.83 (m, 4H), 4.82-4.87 (m, 1H), 7.18 (d, J = 6.8 Hz, 2H), 7.53 (d, J = 6.8 Hz, 1H), 8.05 (d, J = 8.8 Hz, 2H), 8.36 (d, J = 6.8 Hz, 1H), 8.80 (s, 1H), 9.12 (s, 1H). |
| 212 | 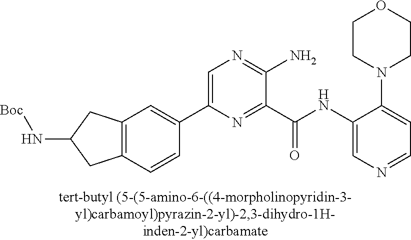 [SMILES: CC(C)(C)OC(=O)NC1Cc2ccc(-c3cnc(N)c(C(=O)Nc4cnccc4N4CCOCC4)n3)cc2C1] | ¹H NMR (400 MHz, Chloroform-d₁) δ: 1.48 (s, 9H), 2.86-2.94 (m, 2H), 3.00-3.03 (m, 4H), 3.34-3.42 (m, 2H), 3.84-3.86 (m, 4H), 4.57 (s, 1H), 4.92 (s, 1H), 7.06 (d, J = 5.2 Hz, 1H), 7.36 (d, J = 8.0 Hz, 1H), 7.80 (d, J = 8.0 Hz, 1H), 7.85 (s, 1H), 8.41 (s, 1H), 8.68 (s, 1H), 9.67 (s, 1H), 10.46 (s, 1H). (two active hydrogens were not found) |
| 213 | 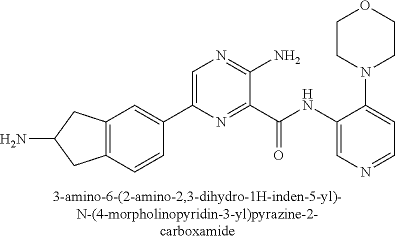 [SMILES: Nc1ncc(-c2ccc3c(c2)CC(N)C3)nc1C(=O)Nc1cnccc1N1CCOCC1] | ¹H NMR (400 MHz, Methanol-d₄) δ: 3.13- 3.19 (m, 2H), 3.47-3.64 (m, 6H), 3.82-3.84 (m, 4H), 4.16-4.22 (m, 1H), 7.46 (d, J = 7.6 Hz, 2H), 7.97-8.04 (m, 2H), 8.37 (d, J = 6.8 Hz, 1H), 8.83 (s, 1H), 9.14 (s, 1H). |
| 214 | 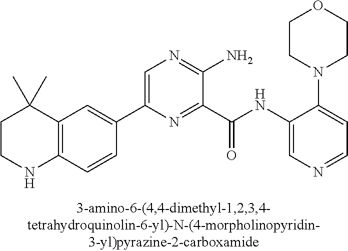 [SMILES: CC1(C)CCNc2ccc(-c3cnc(N)c(C(=O)Nc4cnccc4N4CCOCC4)n3)cc21] | ¹H NMR (400 MHz, Methanol-d₄) δ: 1.34 (s, 6H), 1.75-1.78 (m, 2H), 3.03-3.05 (m, 4H), 3.35-3.37 (m, 2H), 3.83-3.85 (m, 4H), 6.66 (d, J = 8.4 Hz, 1H), 7.24 (d, J = 5.2 Hz, 1H), 7.71-7.77 (m, 2H), 8.27 (d, J = 5.6 Hz, 1H), 8.65 (s, 1H), 9.43 (s, 1H). |
| 215 | 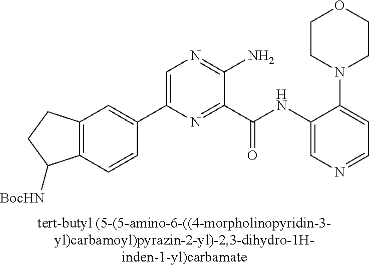 [SMILES: CC(C)(C)OC(=O)NC1CCc2cc(-c3cnc(N)c(C(=O)Nc4cnccc4N4CCOCC4)n3)ccc21] | ¹H NMR (400 MHz, DMSO-d₆) δ: 1.44 (s, 9H), 1.75-1.81 (m, 1H), 2.43-2.49 (m, 2H), 2.83-2.86 (m, 1H), 2.98 (s, 4H), 3.75 (s, 4H), 4.96-5.04 (m, 1H), 7.25-7.40 (m, 3H), 7.68-7.75 (m, 2H), 7.99-8.11 (m, 2H), 8.32 (s, 1H), 8.95 (s, 1H), 9.38 (s, 1H), 10.39 (s, 1H). |
| 216 | 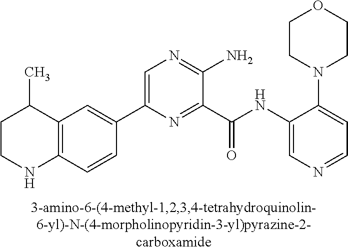 [SMILES: CC1CCNc2ccc(-c3cnc(N)c(C(=O)Nc4cnccc4N4CCOCC4)n3)cc21] | ¹H NMR (400 MHz, Methanol-d₄) δ: 1.36 (d, J = 6.8 Hz, 3H), 1.71-1.76 (m, 1H), 1.94-2.07 (m, 1H), 3.03-3.05 (m, 5H), 3.33- 3.44 (m, 2H), 3.82-3.85 (m, 4H), 6.64 (d, J = 8.4 Hz, 1H), 7.25 (d, J = 4.2 Hz, 1H), 7.62-7.71 (m, 2H), 8.27 (d, J = 5.2 Hz, 1H), 8.65 (s, 1H), 9.46 (s, 1H). |
| 217 | 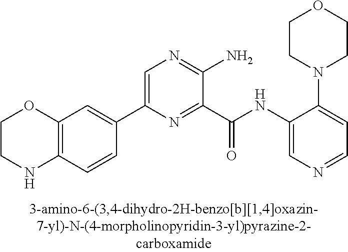 [SMILES: Nc1ncc(-c2ccc3c(c2)OCCN3)nc1C(=O)Nc1cnccc1N1CCOCC1] | ¹H NMR (400 MHz, Methanol-d₄) δ: 3.01- 3.03 (m, 4H), 3.43-3.49 (m, 2H), 3.88-3.98 (m, 4H), 4.23-4.26 (m, 2H), 6.72 (d, J = 8.4 Hz, 1H), 7.26 (d, J = 5.6 Hz, 1H), 7.35-7.47 (m, 1H), 7.55 (s, 1H), 8.27 (d, J = 5.6 Hz, 1H), 8.61 (s, 1H), 9.51 (s, 1H). |
| 218 | 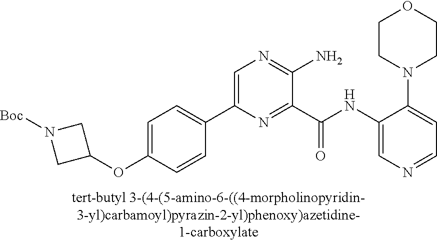 [SMILES: CC(C)(C)OC(=O)N1CC(Oc2ccc(-c3cnc(N)c(C(=O)Nc4cnccc4N4CCOCC4)n3)cc2)C1] | ¹H NMR (400 MHz, Chloroform-d₁) δ: 1.47 (s, 9H), 3.00-3.03 (m, 4H), 3.82-3.85 (m, 4H), 4.06-4.09 (m, 2H), 4.34-4.38 (m, 2H), 4.98-4.99 (m, 1H), 6.89 (d, J = 8.8 Hz, 2H), 7.06 (d, J = 5.2 Hz, 1H), 7.93 (d, J = 8.8 Hz, 2H), 8.40 (d, J = 5.2 Hz, 1H), 8.68 (s, 1H), 9.64 (s, 1H), 10.46 (s, 1H). (two active hydrogens were not found) |
| 219 | 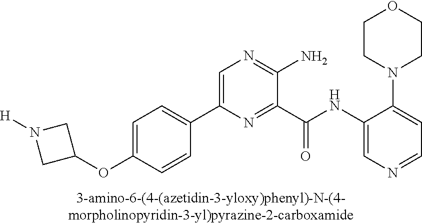 [SMILES: Nc1ncc(-c2ccc(OC3CNC3)cc2)nc1C(=O)Nc1cnccc1N1CCOCC1] | ¹H NMR (400 MHz, Methanol-d₄) δ: 3.71- 3.81 (m, 4H), 3.97-3.99 (m, 4H), 4.18-4.23 (m, 2H), 4.59-4.64 (m, 2H), 5.25-5.29 (m, 1H), 7.04 (d, J = 8.8 Hz, 2H), 7.51 (d, J = 8.0 Hz, 1H), 8.06 (d, J = 8.8 Hz, 2H), 8.36 (d, J = 8.0 Hz, 1H), 8.80 (s, 1H), 9.09 (s, 1H). |
| 220 | 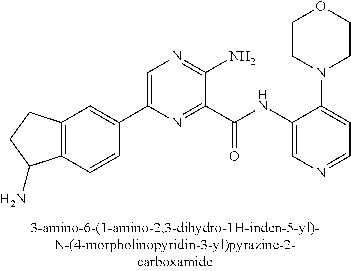 [SMILES: Nc1ncc(-c2ccc3c(c2)CCC3N)nc1C(=O)Nc1cnccc1N1CCOCC1] | ¹H NMR (400 MHz, Methanol-d₄) δ: 2.14- 2.33 (m, 1H), 2.67-2.75 (m, 1H), 3.13-3.18 (m, 1H), 3.29-3.31 (m, 2H), 3.58-3.63 (m, 4H), 3.81-3.85 (m, 4H), 7.53-7.58 (m, 1H), 7.62-7.70 (m, 1H), 8.02-8.10 (m, 1H), 8.15 (s, 1H), 8.37 (d, J = 6.8 Hz, 1H), 8.87 (s, 1H), 9.13 (s, 1H). |
| 221 | 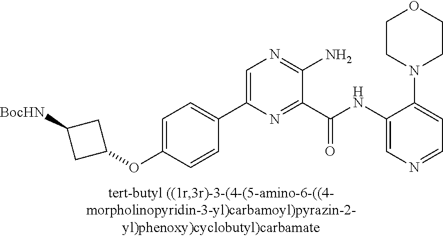 | ¹H NMR (400 MHz, DMSO-d₆) δ: 1.38 (s, 9H), 2.00-2.07 (m, 2H), 2.79-2.80 (m, 2H), 2.95 (s, 4H), 3.71 (s, 5H), 4.45-4.48 (m, 1H), 6.96-6.98 (m, 2H), 7.21-7.24 (m, 2H), 7.66 (s, 2H), 8.06 (d, J = 8.8 Hz, 2H), 8.31 (d, J = 5.2 Hz, 1H), 8.90 (s, 1H), 9.33 (s, 1H), 10.46 (s, 1H). |
| 222 | 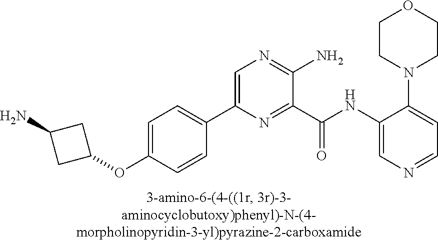 | ¹H NMR (400 MHz, DMSO-d₆ + D₂O) δ: 2.18-2.20 (m, 2H), 2.87-2.89 (m, 2H), 3.42- 3.46 (m, 1H), 3.53 (s, 4H), 3.77 (s, 4H), 4.59-4.62 (m, 1H), 6.96 (d, J = 8.8 Hz, 2H), 7.40 (d, J = 7.2 Hz, 1H), 8.09 (d, J = 8.4 Hz, 2H), 8.32 (d, J = 6.8 Hz, 1H), 8.71 (s, 1H), 8.87 (s, 1H). |
| 223 | 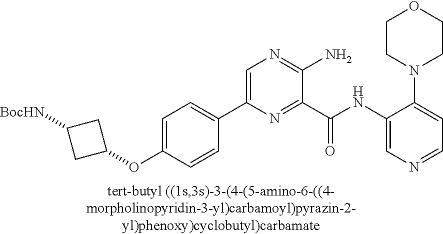 | ¹H NMR (400 MHz, DMSO-d₆) δ: 1.39 (s, 9H), 2.33-2.40 (m, 4H), 2.96 (s, 4H), 3.71 (s, 4H), 4.18 (s, 1H), 4.88 (s, 1H), 6.94 (d, J = 8.8 Hz, 2H), 7.24 (d, J = 4.8 Hz, 2H), 7.67 (s, 2H), 8.07 (d, J = 8.8 Hz, 2H), 8.31-8.32 (m, 1H), 8.90 (s, 1H), 9.33 (s, 1H), 10.47 (s, 1H). |
| 224 | 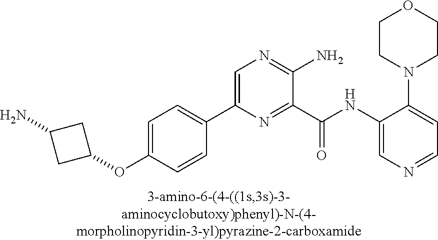 | ¹H NMR (400 MHz, Methanol-d₄) δ: 2.70- 2.75 (m, 4H), 3.52-3.54 (m, 4H), 3.84-3.89 (m, 4H), 4.02-4.06 (m, 1H), 5.06-5.11 (m, 1H), 6.86-6.88 (m, 1H), 7.54-7.56 (m, 1H), 7.59-7.60 (m, 2H), 7.65-7.67 (s, 1H), 8.38 (d, J = 6.8 Hz, 1H), 8.82 (s, 1H), 9.23 (s, 1H). |
| 225 | 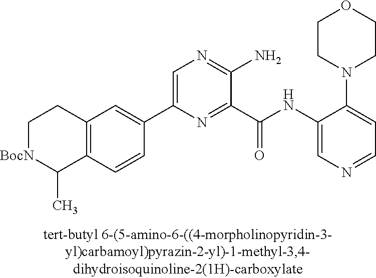 [SMILES: CC1c2ccc(-c3cnc(N)c(C(=O)Nc4cnccc4N4CCOCC4)n3)cc2CCN1C(=O)OC(C)(C)C] | ¹H NMR (300 MHz, DMSO-d₆) δ: 1.38- 1.43 (m, 12H), 2.86-2.98 (m, 6H), 3.15 (s, 1H), 3.68-3.70 (m, 4H), 4.28 (s, 1H), 5.12 (s, 1H), 7.10 (d, J = 5.1 Hz, 1H), 7.33 (d, J = 8.1 Hz, 1H), 7.74 (s, 2H), 7.90-7.93 (m, 2H), 8.29 (d, J = 5.4 Hz, 1H), 8.90 (s, 1H), 9.29 (s, 1H), 10.33 (s, 1H). |
| 226 | 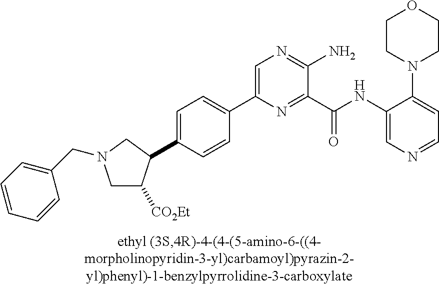 [SMILES: CCOC(=O)[C@@H]1CN(Cc2ccccc2)C[C@H]1c1ccc(-c2cnc(N)c(C(=O)Nc3cnccc3N3CCOCC3)n2)cc1] | ¹H NMR (400 MHz, Methanol-d₄) δ: 1.20- 1.28 (m, 3H), 2.77-2.79 (m, 1H), 3.00-3.34 (m, 8H), 3.65-3.81 (m, 7H), 4.11-4.14 (m, 2H), 7.26-7.50 (m, 8H), 8.02 (d, J = 8.0 Hz, 2H), 8.27 (d, J = 5.6 Hz, 1H), 8.78 (s, 1H), 9.47 (s, 1H). |
| 227 | 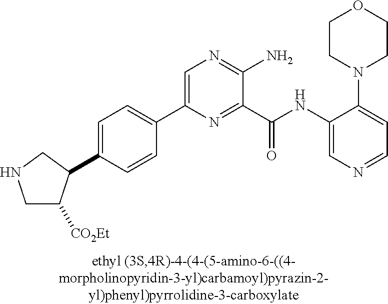 [SMILES: CCOC(=O)[C@@H]1CNC[C@H]1c1ccc(-c2cnc(N)c(C(=O)Nc3cnccc3N3CCOCC3)n2)cc1] | ¹H NMR (300 MHz, Methanol-d₄) δ: 1.13- 1.24 (m, 3H), 2.95 (s, 4H), 3.00-3.18 (m, 2H), 3.31-3.40 (m, 4H), 3.79 (s, 4H), 4.11- 4.18 (m, 2H), 7.25-7.48 (m, 3H), 8.01-8.03 (m, 2H), 8.29 (s, 1H), 8.75 (s, 1H), 9.43 (s, 1H). |
| 228 | 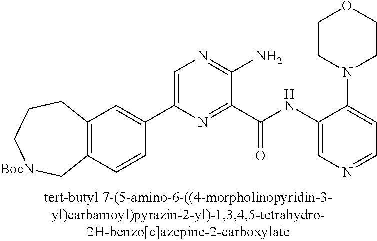 [SMILES: CC(C)(C)OC(=O)N1CCCc2cc(-c3cnc(N)c(C(=O)Nc4cnccc4N4CCOCC4)n3)ccc2C1] | ¹H NMR (300 MHz, Methanol-d₄) δ: 1.42 (s, 9H), 1.84 (s, 2H), 3.04-3.10 (m, 4H), 3.14-3.17 (m, 2H), 3.77-3.83 (m, 6H), 4.51 (s, 2H), 7.26 (d, J = 5.4 Hz, 1H), 7.37-7.43 (m, 1H), 7.78-7.87 (m, 2H), 8.28 (d, J = 5.4 Hz, 1H), 8.78 (d, J = 8.4 Hz, 1H), 9.49 (s, 1H). |
| 229 | 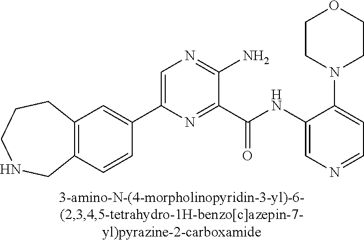 [SMILES: Nc1ncc(-c2ccc3c(c2)CCCNC3)nc1C(=O)Nc1cnccc1N1CCOCC1] | ¹H NMR (300 MHz, Methanol-d₄) δ: 2.10 (s, 2H), 3.15-3.33 (m, 2H), 3.68-3.74 (m, 6H), 3.81-3.91 (m, 4H), 4.48 (s, 2H), 7.51- 7.64 (m, 2H), 7.99-8.11 (m, 1H), 8.15 (s, 1H), 8.37 (d, J = 7.2 Hz, 1H), 8.90 (s, 1H), 9.14 (s, 1H). |
| 230 | 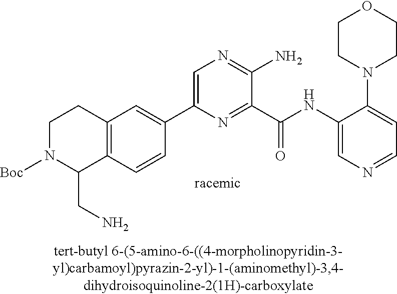 [SMILES: CC(C)(C)OC(=O)N1CCc2cc(-c3cnc(N)c(C(=O)Nc4cnccc4N4CCOCC4)n3)ccc2C1CN] | ¹H NMR (300 MHz, Acetonitrile-d₃) δ: 1.49 (s, 9H), 2.14 (s, 2H), 2.85-3.00 (m, 8H), 3.34 (s, 1H), 3.71-3.75 (m, 4H), 4.09 (s, 1H), 5.07 (s, 1H), 7.12 (d, J = 5.4 Hz, 1H), 7.35 (d, J = 8.1 Hz, 1H), 7.81 (s, 1H), 7.87 (d, J = 8.1 Hz, 1H), 8.31 (d, J = 5.1 Hz, 1H), 8.74 (s, 1H), 9.41 (s, 1H), 10.34 (s, 1H). (two active hydrogens were not found) |
| 231 | 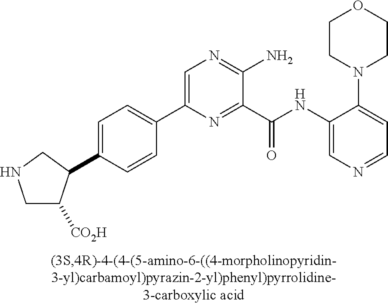 [SMILES: Nc1ncc(-c2ccc([C@@H]3CNC[C@H]3C(=O)O)cc2)nc1C(=O)Nc1cnccc1N1CCOCC1] | ¹H NMR (300 MHz, DMSO-d₆) δ: 1.21 (s, 1H), 2.88 (s, 6H), 3.60-3.98 (m, 8H), 7.21 (s, 1H), 7.46 (s, 2H), 7.73 (s, 2H), 8.07 (s, 2H), 8.31 (s, 1H), 8.93 (s, 1H), 9.28 (s, 1H), 10.38 (s, 1H). (one active hydrogen was not found) |
| 232 | 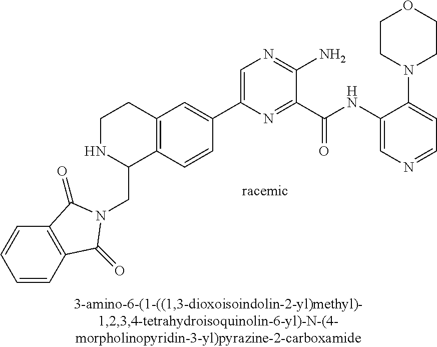 [SMILES: Nc1ncc(-c2ccc3c(c2)CCNC3CN2C(=O)c3ccccc3C2=O)nc1C(=O)Nc1cnccc1N1CCOCC1] | ¹H NMR (300 MHz, Acetonitrile-d₃ + D₂O) δ: 3.18-3.27 (m, 2H), 3.44-3.49 (m, 5H), 3.63-3.77 (m, 5H), 4.11-4.34 (m, 2H), 4.92-4.94 (m, 1H), 7.30 (d, J = 6.6 Hz, 1H), 7.59 (d, J = 8.7 Hz, 1H), 7.83-7.96 (m, 6H), 8.18-8.21 (m, 1H), 8.74-8.81 (m, 2H). |
| 233 | 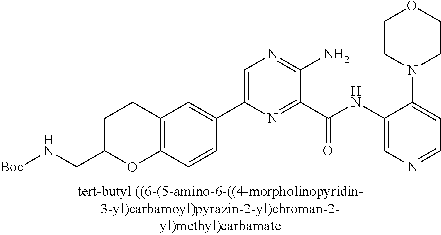 [SMILES: CC(C)(C)OC(=O)NCC1CCc2cc(-c3cnc(N)c(C(=O)Nc4cnccc4N4CCOCC4)n3)ccc2O1] | ¹H NMR (400 MHz, Chloroform-d₁) δ: 1.57 (s, 9H), 1.85-2.10 (m, 3H), 2.87-3.04 (m, 5H), 3.33-3.39 (m, 1H), 3.59-3.66 (m, 1H), 3.85-3.86 (m, 4H), 4.18 (s, 1H), 5.10 (s, 1H), 6.96 (d, J = 8.8 Hz, 1H), 7.06 (d, J = 5.2 Hz, 1H), 7.64 (s, 1H), 7.75-7.78 (m, 1H), 8.39 (d, J = 4.4 Hz, 1H), 8.66 (s, 1H), 9.64 (d, J = 4.4 Hz, 1H), 10.40 (s, 1H). (two active hydrogens were not found) |
| 234 | 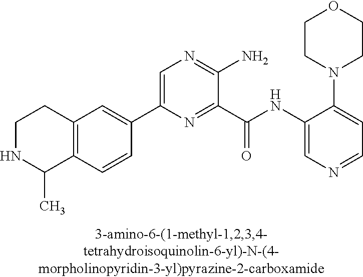 [SMILES: CC1NCCc2cc(-c3cnc(N)c(C(=O)Nc4cnccc4N4CCOCC4)n3)ccc21] | ¹H NMR (300 MHz, DMSO-d₆ + D₂O) δ: 1.61 (d, J = 6.9 Hz, 3H), 3.09-3.14 (m, 2H), 3.30-3.40 (m, 1H), 3.51-3.53 (m, 2H), 3.60- 3.63 (m, 7H), 4.62-4.64 (m, 1H), 7.41-7.44 (m, 2H), 8.08-8.12 (m, 2H), 8.35-8.38 (m, 1H), 8.71 (s, 1H), 8.98 (s, 1H). |
| 235 | 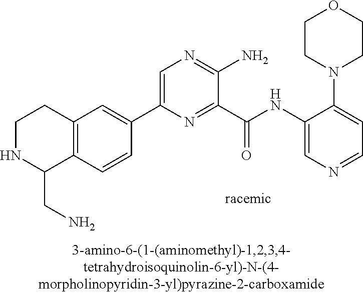 [SMILES: NCC1NCCc2cc(-c3cnc(N)c(C(=O)Nc4cnccc4N4CCOCC4)n3)ccc21] | ¹H NMR (300 MHz, Acetonitrile-d₃ + D₂O) δ: 3.18-3.19 (m, 2H), 3.48-3.73 (m, 12H), 4.97-5.01 (m, 1H), 7.32 (d, J = 7.2 Hz, 1H), 7.45 (d, J = 8.1 Hz, 1H), 7.89-7.98 (m, 2H), 8.19-8.22 (m, 1H), 8.71-8.78 (m, 2H). |
| 236 | 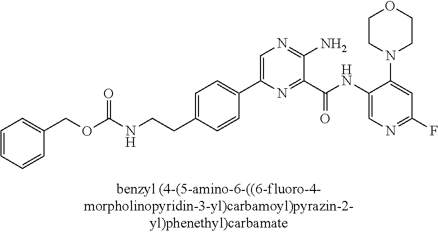 [SMILES: Nc1ncc(-c2ccc(CCNC(=O)OCc3ccccc3)cc2)nc1C(=O)Nc1cnc(F)cc1N1CCOCC1] | [M + H]⁺: 572.3 |
| 237 | 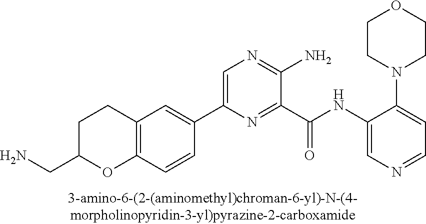 [SMILES: NCC1CCc2cc(-c3cnc(N)c(C(=O)Nc4cnccc4N4CCOCC4)n3)ccc2O1] | ¹H NMR (400 MHz, Methanol-d₄) δ: 2.05- 2.08 (m, 1H), 2.09-2.10 (m, 1H), 2.87-3.00 (m, 8H), 3.80-3.82 (m, 4H), 4.05-4.10 (m, 1H), 6.91 (d, J = 5.2 Hz, 1H), 7.23 (d, J = 5.2 Hz, 1H), 7.75-7.78 (m, 2H), 8.27 (s, 1H), 8.64 (s, 1H), 9.47 (s, 1H). |
| 238 | 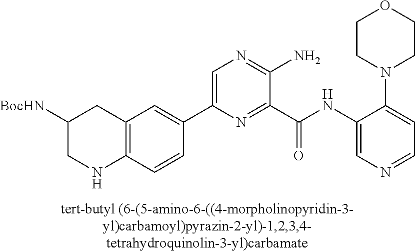 [SMILES: CC(C)(C)OC(=O)NC1CNc2ccc(-c3cnc(N)c(C(=O)Nc4cnccc4N4CCOCC4)n3)cc2C1] | ¹H NMR (400 MHz, Methanol-d₄) δ: 1.38 (s, 9H), 2.74-2.78 (m, 1H), 2.93 (s, 5H), 3.01 (s, 1H), 3.36-3.42 (m, 1H), 3.78 (s, 4H), 3.94 (s, 1H), 6.57-6.59 (m, 1H), 7.18- 7.19 (m, 1H), 7.40-7.61 (m, 2H), 8.23 (s, 1H), 8.43-8.49 (m, 1H), 9.42 (s, 1H). |
| 239 | 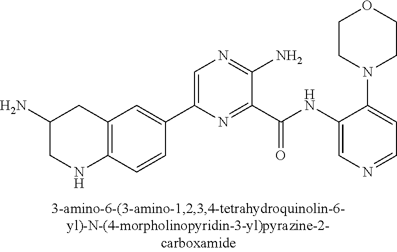 [SMILES: Nc1ncc(-c2ccc3c(c2)CC(N)CN3)nc1C(=O)Nc1cnccc1N1CCOCC1] | ¹H NMR (400 MHz, DMSO-d₆) δ: 1.23 (s, 2H), 2.67-2.73 (m, 1H), 2.97-3.06 (m, 7H), 3.71 (s, 7H), 6.22 (s, 1H), 6.62 (d, J = 8.4 Hz, 1H), 7.23-7.24 (m, 1H), 7.54 (s, 2H), 7.57-7.78 (m, 2H), 8.31-8.33 (m, 2H), 8.83 (s, 1H), 9.35 (s, 1H), 10.38 (s, 1H). |
| 240 | 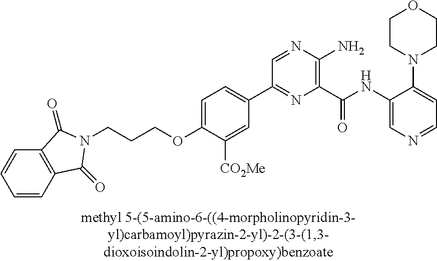 [SMILES: COC(=O)c1cc(-c2cnc(N)c(C(=O)Nc3cnccc3N3CCOCC3)n2)ccc1OCCCN1C(=O)c2ccccc2C1=O] | [M + H]⁺: 638.3 |
| 241 | 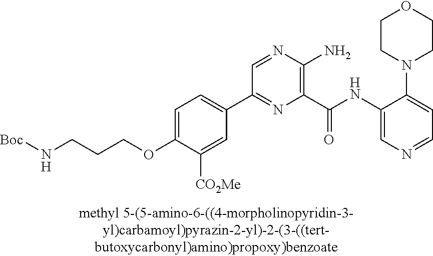 [SMILES: COC(=O)c1cc(-c2cnc(N)c(C(=O)Nc3cnccc3N3CCOCC3)n2)ccc1OCCCNC(=O)OC(C)(C)C] | [M + Na]⁺: 630.3 [M − Boc]⁺: 506.2 |
| 242 | 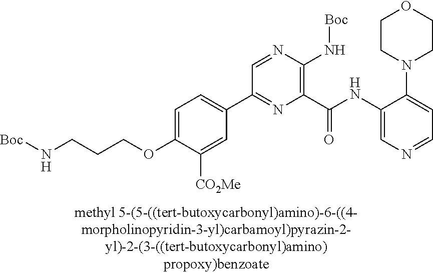 [SMILES: COC(=O)c1cc(-c2cnc(NC(=O)OC(C)(C)C)c(C(=O)Nc3cnccc3N3CCOCC3)n2)ccc1OCCCNC(=O)OC(C)(C)C] | [M + Na]⁺: 730.3 [M − Boc]⁺: 606.2 |
| 243 | 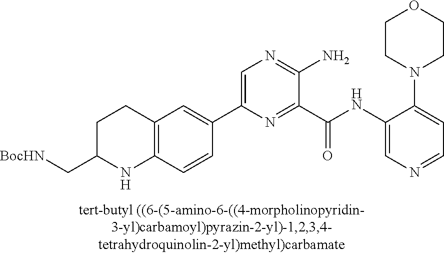 [SMILES: CC(C)(C)OC(=O)NCC1CCc2cc(-c3cnc(N)c(C(=O)Nc4cnccc4N4CCOCC4)n3)ccc2N1] | ¹H NMR (400 MHz, DMSO-d₆) δ: 1.54 (s, 9H), 1.88 (s, 1H), 2.76 (s, 2H), 2.96 (s, 6H), 3.04 (s, 1H), 3.76 (s, 4H), 6.04 (s, 1H), 6.58 (d, J = 8.0 Hz, 1H), 6.80 (s, 1H), 7.04 (s, 1H), 7.22 (s, 1H), 7.23 (s, 1H), 7.51 (s, 1H), 7.64-7.72 (m, 2H), 8.32 (s, 1H), 8.79 (s, 1H), 9.37 (s, 1H), 10.38 (s, 1H). |
| 244 | 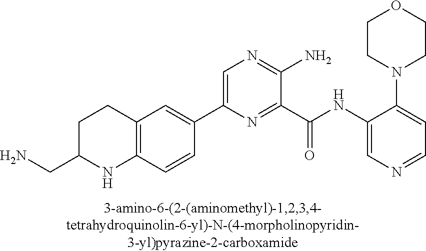 [SMILES: NCC1CCc2cc(-c3cnc(N)c(C(=O)Nc4cnccc4N4CCOCC4)n3)ccc2N1] | ¹H NMR (400 MHz, Methanol-d₄) δ: 1.75- 1.72 (m, 1H), 2.01-2.05 (m, 1H), 2.76-3.01 (m, 4H), 3.14-3.15 (m, 4H), 3.49-3.50 (m, 1H), 3.80-3.88 (m, 4H), 6.69 (d, J = 8.4 Hz, 1H), 7.27 (d, J = 5.6 Hz, 1H), 7.63- 7.69 (m, 2H), 8.29 (d, J = 4.4 Hz, 1H), 8.66 (s, 1H), 9.50 (s, 1H). |
| 245 | 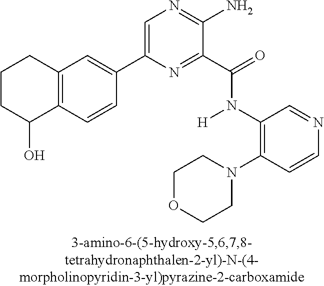 [SMILES: Nc1ncc(-c2ccc3c(c2)CCCC3O)nc1C(=O)Nc1cnccc1N1CCOCC1] | ¹H NMR (400 MHz, DMSO-d₆) δ: 1.70- 1.73 (m, 2H), 1.93-1.96 (m, 2H), 2.78-2.83 (m, 2H), 2.97-2.99 (m, 4H), 3.74-3.76 (m, 4H), 4.60-4.63 (m, 1H), 5.21 (d, J = 5.6 Hz, 1H), 7.23 (d, J = 5.6 Hz, 1H), 7.54 (d, J = 8.0 Hz, 1H), 7.74 (s, 2H), 7.83 (s, 1H), 7.95 (d, J = 8.0 Hz, 1H), 8.31 (d, J = 5.2 Hz, 1H), 8.94 (s, 1H), 9.33 (s, 1H), 10.33 (s, 1H). |
| 246 | 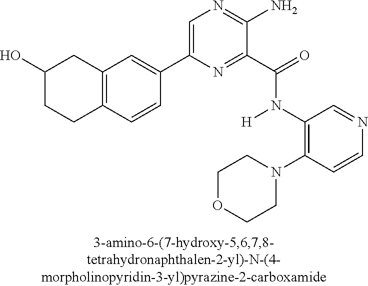 [SMILES: Nc1ncc(-c2ccc3c(c2)CC(O)CC3)nc1C(=O)Nc1cnccc1N1CCOCC1] | ¹H NMR (400 MHz, DMSO-d₆ + D₂O) δ: 1.65 (s, 1H), 1.98 (s, 1H), 2.91-2.95 (m, 2H), 2.97-3.01 (m, 5H), 3.08 (d, J = 5.6 Hz, 1H), 3.73 (s, 4H), 4.01 (s, 1H), 7.21- 7.25 (m, 2H), 7.68-7.81 (m, 2H), 8.30 (s, 1H), 8.91 (d, J = 4.0 Hz, 1H), 9.28 (d, J = 3.6 Hz, 1H). |
| 247 | 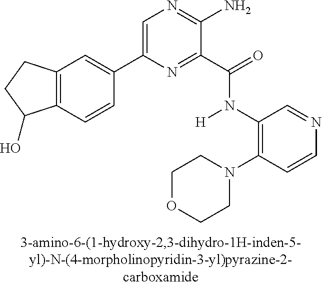 [SMILES: Nc1ncc(-c2ccc3c(c2)CCC3O)nc1C(=O)Nc1cnccc1N1CCOCC1] | ¹H NMR (300 MHz, Methanol-d₄) δ: 1.96- 2.05 (m, 1H), 2.46-2.54 (m, 1H), 2.78-2.97 (m, 1H), 3.02-3.11 (m, 4H), 3.12-3.18 (m, 1H), 3.82-3.85 (m, 4H), 5.21-5.26 (m, 1H), 7.27 (d, J = 5.7 Hz, 1H), 7.53 (d, J = 7.2 Hz 1H), 7.91-7.96 (m, 2H), 8.28 (d, J = 5.1 Hz, 1H), 8.78 (s, 1H), 9.56 (s, 1H). |
| 248 | 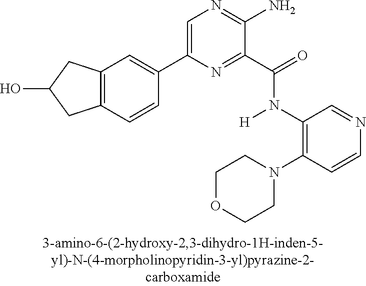 [SMILES: Nc1ncc(-c2ccc3c(c2)CC(O)C3)nc1C(=O)Nc1cnccc1N1CCOCC1] | ¹H NMR (300 MHz, DMSO-d₆) δ: 2.77- 2.83 (m, 2H), 2.87-3.10 (m, 4H), 3.12-3.20 (m, 2H), 3.74-3.76 (m, 4H), 4.56 (s, 1H), 4.93 (s, 1H), 7.25 (d, J = 5.4 Hz, 1H), 7.34 (d, J = 7.5 Hz, 1H), 7.66 (s, 2H), 7.88-8.05 (m, 2H), 8.32 (d, J = 5.4 Hz, 1H), 8.94 (s, 1H), 9.38 (s, 1H), 10.41 (s, 1H). |
| 249 | 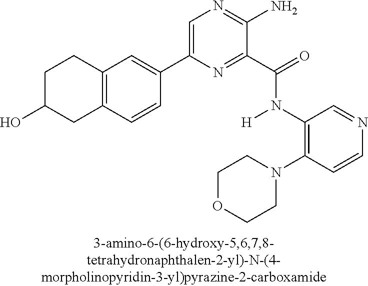 [SMILES: Nc1ncc(-c2ccc3c(c2)CCC(O)C3)nc1C(=O)Nc1cnccc1N1CCOCC1] | ¹H NMR (400 MHz, DMSO-d₆) δ: 1.60- 1.75 (m, 1H), 1.90-2.00 (m, 1H), 2.49-2.50 (m, 1H), 2.60-2.67 (m, 1H), 2.96-3.02 (m, 6H), 3.73-3.75 (m, 4H), 3.90-4.00 (m, 1H), 4.81-4.82 (s, 1H), 7.18-7.23 (m, 2H), 7.70 (s, 2H), 7.83-7.88 (m, 2H), 8.31-8.32 (m, 1H), 8.92 (s, 1H), 9.33 (s, 1H), 10.39 (s, 1H). |
| 250 | 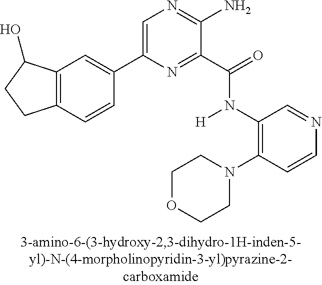 [SMILES: Nc1ncc(-c2ccc3c(c2)C(O)CC3)nc1C(=O)Nc1cnccc1N1CCOCC1] | ¹H NMR (300 MHz, DMSO-d₆) δ: 1.81- 1.91 (m, 1H), 2.28-2.35 (m, 1H), 2.73-2.79 (m, 1H), 2.81-3.03 (m, 5H), 3.72 (s, 4H), 5.10-5.14 (m, 1H), 5.16-5.30 (m, 1H), 7.23 (d, J = 5.1 Hz, 1H), 7.36 (d, J = 7.8 Hz, 1H), 7.73 (s, 2H), 7.97-8.07 (m, 2H), 8.31 (d, J = 5.4 Hz, 1H), 8.94 (s, 1H), 9.35 (s, 1H), 10.39 (s, 1H). |
| 251 | 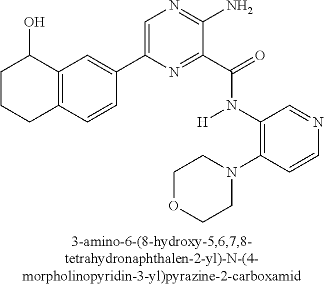 [SMILES: Nc1ncc(-c2ccc3c(c2)C(O)CCC3)nc1C(=O)Nc1cnccc1N1CCOCC1] | ¹H NMR (400 MHz, DMSO-d₆) δ: 1.72 (d, J = 8.0 Hz, 2H), 1.93 (s, 2H), 2.70-2.82 (m, 2H), 2.94-3.32 (m, 4H), 3.72 (s, 4H), 4.67 (s, 1H), 5.16 (d, J = 5.2 Hz, 1H), 7.20 (d, J = 6.8 Hz, 2H), 7.53-7.72 (m, 2H), 7.94 (d, J = 8.0 Hz, 1H), 8.10 (s, 1H), 8.31 (d, J = 4.8 Hz, 1H), 8.89 (s, 1H), 9.31 (s, 1H), 10.33 (s, 1H). |
| 252 | 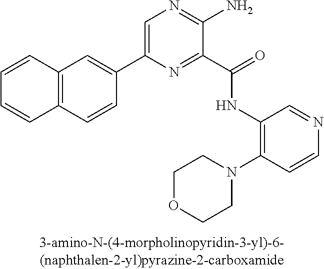 [SMILES: Nc1ncc(-c2ccc3ccccc3c2)nc1C(=O)Nc1cnccc1N1CCOCC1] | ¹H NMR (400 MHz, DMSO-d₆) δ: 2.98- 3.00 (m, 4H), 3.71-3.73 (m, 4H), 7.24 (d, J = 5.2 Hz, 1H), 7.54-7.60 (m, 2H), 7.82 (s, 2H), 7.98-8.08 (m, 3H), 8.32-8.36 (m, 2H), 8.67 (s, 1H), 9.13 (s, 1H), 9.33 (s, 1H), 10.47 (s, 1H). |
| 253 | 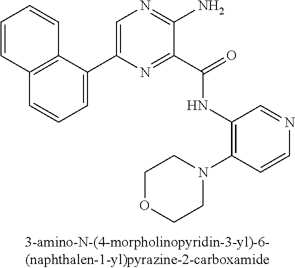 [SMILES: Nc1ncc(-c2cccc3ccccc23)nc1C(=O)Nc1cnccc1N1CCOCC1] | ¹H NMR (300 MHz, DMSO-d₆) δ: 2.73- 2.75 (m, 4H), 2.97-3.00 (m, 4H), 7.13 (d, J = 5.4 Hz, 1H), 7.52-7.74 (m, 3H), 7.81 (s, 3H), 8.05-8.03 (m, 3H), 8.27 (d, J = 5.1 Hz, 1H), 8.65 (s, 1H), 9.47 (s, 1H), 10.40 (s, 1H). |
| 254 | 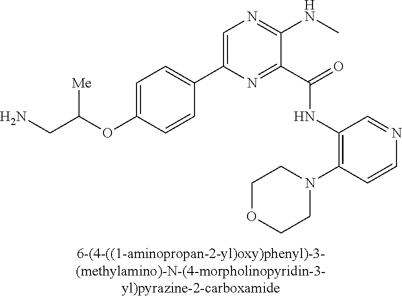 [SMILES: CNc1ncc(-c2ccc(OC(C)CN)cc2)nc1C(=O)Nc1cnccc1N1CCOCC1] | ¹H NMR (400 MHz, Methanol-d₄) δ: 1.39 (s, 3H), 2.98-3.32 (m, 6H), 3.07 (s, 3H), 3.72-3.77 (m, 4H), 4.53-4.59 (m, 1H), 7.02 (d, J = 8.8 Hz, 2H), 7.07 (s, 1H), 7.85 (d, J = 8.8 Hz, 2H), 7.81 (s, 1H), 8.86 (s, 1H), 9.44 (s, 1H). |
| 255 | 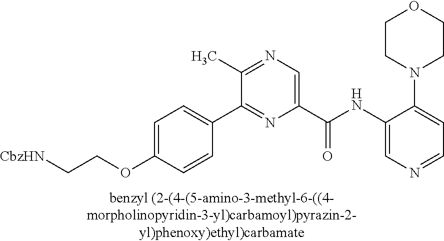 [SMILES: Cc1ncc(C(=O)Nc2cnccc2N2CCOCC2)nc1-c1ccc(OCCNC(=O)OCc2ccccc2)cc1] | ¹H NMR (300 MHz, DMSO-d₆) δ: 2.50 (s, 3H), 2.86 (s, 4H), 3.42-3.51 (m, 6H), 4.07 (s, 2H), 5.05 (s, 2H), 7.06 (d, J = 7.8 Hz, 2H), 7.17-7.18 (m, 1H), 7.35 (s, 5H), 7.54- 7.62 (m, 5H), 8.27 (d, J = 4.2 Hz, 1H), 9.41 (s, 1H), 10.33 (s, 1H). |
| 256 | 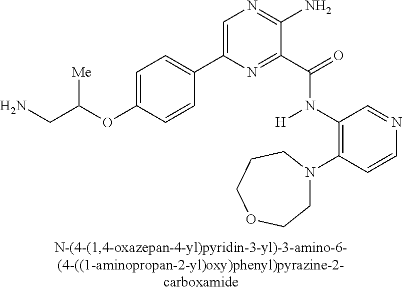 [SMILES: CC(CN)Oc1ccc(-c2cnc(N)c(C(=O)Nc3cnccc3N3CCCOCC3)n2)cc1] | ¹H NMR (400 MHz, Methanol-d₄) δ: 1.28- 1.31 (m, 3H), 1.95-2.01 (m, 2H), 2.90 (d, J = 5.6 Hz, 2H), 3.31-3.38 (m, 4H), 3.71 (t, J = 6.0 Hz, 2H), 3.79-3.81 (m, 2H), 4.50-4.57 (m, 1H), 7.03-7.16 (m, 3H), 7.90- 7.94 (m, 2H), 8.17 (d, J = 5.6 Hz, 1H), 8.56 (s, 1H), 9.04 (s, 1H). |
| 257 | 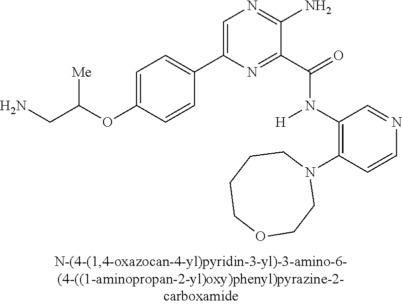 [SMILES: CC(CN)Oc1ccc(-c2cnc(N)c(C(=O)Nc3cnccc3N3CCCCOCC3)n2)cc1] | ¹H NMR (400 MHz, DMSO-d₆ + D₂O) δ: 1.09 (d, J = 6.4 Hz, 3H), 1.69 (s, 4H), 3.10 (s, 2H), 3.16-3.17 (m, 2H), 3.63 (s, 2H), 3.72-3.74 (m, 2H), 4.72-4.74 (m, 3H), 7.15 (d, J = 9.2 Hz, 2H), 7.64 (d, J = 6.4 Hz, 1H), 8.08 (d, J = 8.8 Hz, 2H), 8.63 (d, J = 6.4 Hz, 1H), 8.93 (s, 1H), 9.35 (s, 1H). |
| 258 | 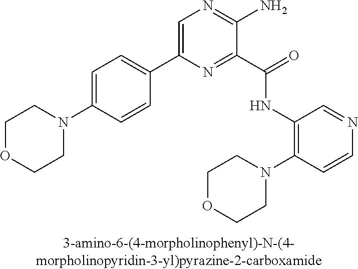 [SMILES: Nc1ncc(-c2ccc(N3CCOCC3)cc2)nc1C(=O)Nc1cnccc1N1CCOCC1] | ¹H NMR (400 MHz, DMSO-d₆) δ: 2.96- 3.03 (m, 4H), 3.21-3.22 (m, 4H), 3.70-3.78 (m, 8H), 7.06 (d, J = 9.2 Hz, 2H), 7.24 (d, J = 5.6 Hz, 1H), 7.61 (s, 1H), 8.03 (d, J = 8.8 Hz, 2H), 8.29-8.32 (s, 2H), 8.90 (s, 1H), 9.33 (s, 1H), 10.45 (s, 1H). |
| 259 | 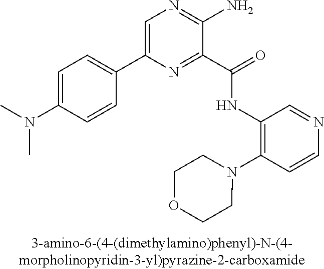 [SMILES: CN(C)c1ccc(-c2cnc(N)c(C(=O)Nc3cnccc3N3CCOCC3)n2)cc1] | ¹H NMR (400 MHz, DMSO-d₆) δ: 2.50- 2.51 (s, 1H), 2.95-2.98 (m, 9H), 3.73-3.75 (s, 4H), 6.81 (d, J = 8.8 Hz, 2H), 7.23 (d, J = 5.2 Hz, 1H), 7.54 (s, 1H), 7.98 (d, J = 8.8 Hz, 2H), 8.17 (s, 1H), 8.25 (d, J = 4.4 Hz, 1H), 8.85 (s, 1H), 9.35 (s, 1H), 10.45 (s, 1H). |
| 260 | 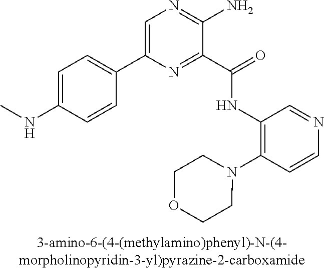 [SMILES: CNc1ccc(-c2cnc(N)c(C(=O)Nc3cnccc3N3CCOCC3)n2)cc1] | ¹H NMR (400 MHz, DMSO-d₆) δ: 2.67- 2.74 (m, 3H), 2.90-3.01 (m, 4H), 3.75-3.77 (m, 4H), 6.05 (s, 1H), 6.65 (d, J = 8.8 Hz, 2H), 7.25 (d, J = 5.2 Hz, 1H), 7.51 (s, 1H), 7.93 (d, J = 8.8 Hz, 2H), 8.28-8.32 (m, 2H), 8.84 (s, 1H), 9.38 (s, 1H), 10.55 (s, 1H). |
| 261 | 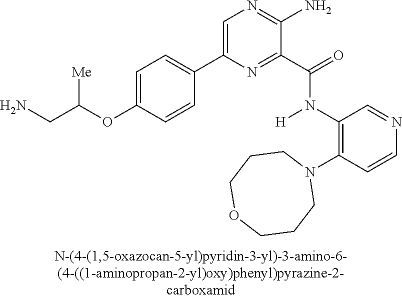 [SMILES: CC(CN)Oc1ccc(-c2cnc(N)c(C(=O)Nc3cnccc3N3CCCOCCC3)n2)cc1] | ¹H NMR (400 MHz, Methanol-d₄) δ: 1.37 (d, J = 6.0 Hz, 3H), 1.90-1.94 (m, 2H), 3.15-3.17 (m, 1H), 3.18-3.20 (m, 1H), 3.53-3.57 (m, 4H), 3.88-3.90 (m, 2H), 4.81-4.83 (m, 2H), 5.03-5.20 (m, 2H), 5.76-5.81 (m, 1H), 7.10-7.14 (m, 3H), 8.10 (d, J = 8.8 Hz, 2H), 8.24 (s, 1H), 8.25 (s, 1H), 8.77 (s, 1H). |
| 262 | 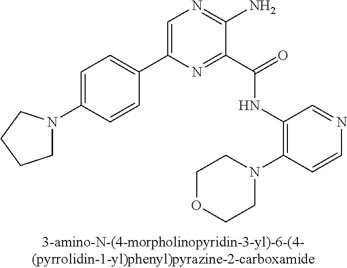 [SMILES: Nc1ncc(-c2ccc(N3CCCC3)cc2)nc1C(=O)Nc1cnccc1N1CCOCC1] | ¹H NMR (400 MHz, DMSO-d₆) δ: 1.97- 2.00 (m, 4H), 2.95-3.01 (m, 4H), 3.32-3.47 (s, 4H), 3.67-3.76 (m, 4H), 6.64 (d, J = 8.8 Hz, 2H), 7.24 (d, J = 5.6 Hz, 1H), 7.45 (s, 2H), 7.98 (d, J = 8.8 Hz, 2H), 8.15-8.32 (m, 1H), 8.85 (s, 1H), 9.36 (s, 1H), 10.47 (s, 1H). |
| 263 | 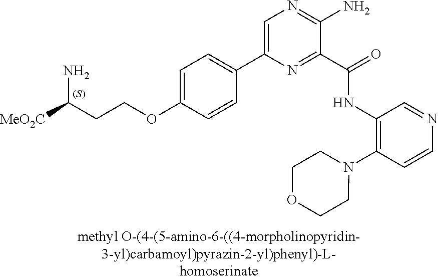 | ¹H NMR (400 MHz, Methanol-d₄) δ: 2.05- 2.27 (m, 2H), 2.97-3.02 (m, 4H), 3.71-3.75 (m, 4H), 3.78-3.86 (m, 4H), 4.15-4.19 (m, 2H), 7.02 (d, J = 4.8 Hz, 2H), 7.22 (d, J = 5.6 Hz, 1H), 7.93 (d, J = 4.8 Hz, 2H), 8.25 (d, J = 5.6 Hz, 1H), 8.66 (s, 1H), 9.46 (s, 1H). |
| 264 | 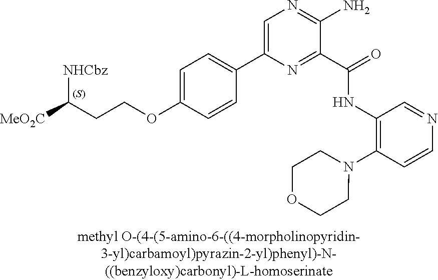 | ¹H NMR (400 MHz, DMSO-d₆) δ: 2.12- 2.20 (m, 1H), 2.22-2.23 (m, 1H), 2.96 (s, 4H), 3.61-3.71 (m, 7H), 4.06-4.16 (m, 2H), 4.30-4.35 (m, 1H), 5.04 (s, 2H), 7.05 (d, J = 8.8 Hz, 2H), 7.22-7.38 (m, 6H), 7.66 (s, 2H), 7.89 (d, J = 7.6 Hz, 1H), 8.08 (d, J = 8.8 Hz, 2H), 8.31 (d, J = 5.2 Hz, 1H), 8.91 (s, 1H), 9.33 (s, 1H), 10.44 (s, 1H). |
| 265 | 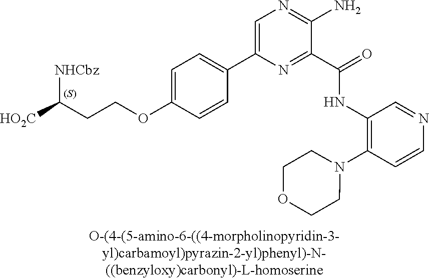 | ¹H NMR (400 MHz, DMSO-d₆) δ: 1.89- 2.16 (m, 1H), 2.22-2.32 (m, 1H), 2.95-2.97 (m, 4H), 3.71-3.73 (m, 4H), 4.09-4.22 (m, 3H), 5.03 (s, 2H), 7.05 (d, J = 8.8 Hz, 2H), 7.22-7.35 (m, 6H), 7.59-7.67 (m, 3H), 8.08 (d, J = 8.4 Hz, 2H), 8.31 (d, J = 5.2 Hz, 1H), 8.91 (s, 1H), 9.34 (s, 1H), 10.85 (s, 1H), 12.83 (s, 1H). |
| 266 | 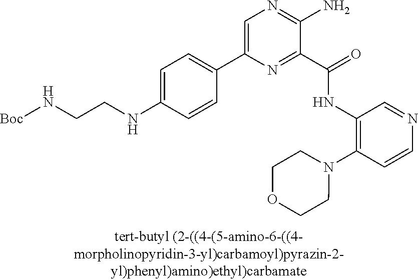 [SMILES: CC(C)(C)OC(=O)NCCNc1ccc(-c2cnc(N)c(C(=O)Nc3cnccc3N3CCOCC3)n2)cc1] | ¹H NMR (400 MHz, Methanol-d₄) δ: 1.46 (s, 9H), 2.91-2.93 (m, 4H), 3.22-3.31 (m, 4H), 3.77-3.79 (m, 4H), 6.67 (d, J = 8.8 Hz, 2H), 7.17 (d, J = 5.2 Hz, 1H), 7.75 (d, J = 8.8 Hz, 2H), 8.22 (d, J = 5.2 Hz, 1H), 8.52 (s, 1H), 9.44 (s, 1H). |
| 267 | 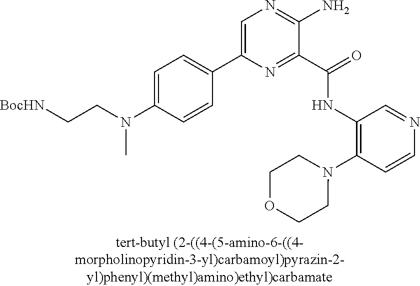 [SMILES: CN(CCNC(=O)OC(C)(C)C)c1ccc(-c2cnc(N)c(C(=O)Nc3cnccc3N3CCOCC3)n2)cc1] | ¹H NMR (400 MHz, Methanol-d₄) δ: 1.42 (s, 9H), 2.98-3.00 (m, 4H), 3.03 (s, 3H), 3.24-3.27 (m, 2H), 3.48-3.51 (m, 2H), 3.82- 3.84 (m, 4H), 6.86 (d, J = 8.8 Hz, 2H), 7.23 (d, J = 5.6 Hz, 1H), 7.86 (d, J = 8.8 Hz, 2H), 8.25 (d, J = 5.6 Hz, 1H), 8.62 (s, 1H), 9.46 (s, 1H). |
| 268 | 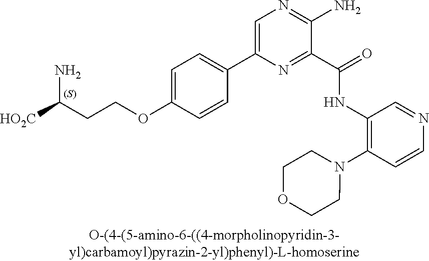 | ¹H NMR (400 MHz, Methanol-d₄) δ: 2.30- 2.53 (m, 2H), 3.38-3.40 (m, 4H), 3.74-3.81 (m, 4H), 4.00-4.03 (m, 1H), 4.26-4.33 (m, 2H), 7.11 (d, J = 8.8 Hz, 2H), 7.44 (d, J = 6.4 Hz, 1H), 8.00 (d, J = 8.8 Hz, 2H), 8.32 (d, J = 6.4 Hz, 1H), 8.75 (s, 1H), 9.20 (s, 1H). |
| 269 | 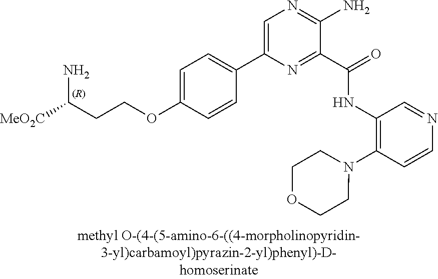 | ¹H NMR (400 MHz, Methanol-d₄) δ: 2.15- 2.27 (m, 2H), 2.99-3.02 (m, 4H), 3.70-3.74 (m, 4H), 3.79-3.82 (m, 4H), 4.15-4.20 (m, 2H), 7.04 (d, J = 8.8 Hz, 2H), 7.24 (d, J = 5.6 Hz, 1H), 7.97 (d, J = 8.8 Hz, 2H), 8.26 (d, J = 5.6 Hz, 1H), 8.69 (s, 1H), 9.47 (s, 1H). |
| 270 | 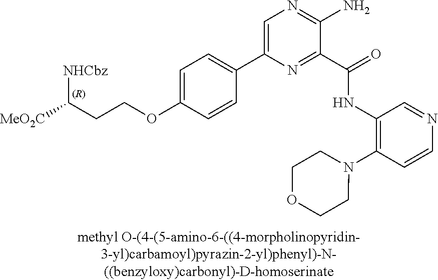 | ¹H NMR (400 MHz, DMSO-d₆) δ: 2.08- 2.23 (m, 2H), 2.96 (s, 4H), 3.60-3.71 (m, 7H), 4.08-4.16 (m, 2H), 4.29-4.35 (s, 1H), 5.04 (s, 2H), 7.05 (d, J = 8.8 Hz, 2H), 7.23- 7.36 (m, 6H), 7.67 (s, 2H), 7.89 (d, J = 8.0 Hz, 1H), 8.09 (d, J = 8.4 Hz, 2H), 8.31 (d, J = 5.2 Hz, 1H), 8.92 (s, 1H), 9.33 (s, 1H), 10.44 (s, 1H). |
| 271 | 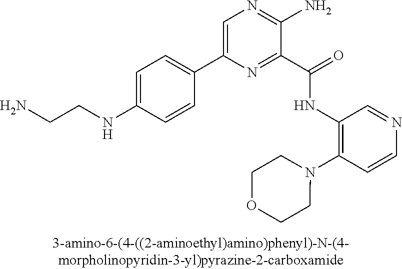 [SMILES: NCCNc1ccc(-c2cnc(N)c(C(=O)Nc3cnccc3N3CCOCC3)n2)cc1] | ¹H NMR (400 MHz, Methanol-d₄) δ: 2.81- 2.98 (m, 4H), 3.02-3.06 (m, 2H), 3.35-3.40 (m, 2H), 3.64-3.82 (m, 4H), 6.77 (d, J = 9.0 Hz, 2H), 7.22 (d, J = 6.0 Hz, 1H), 7.83 (d, J = 6.0 Hz, 2H), 8.25 (d, J = 6.0 Hz, 1H), 8.59 (s, 1H), 9.45 (s, 1H). |
| 272 | 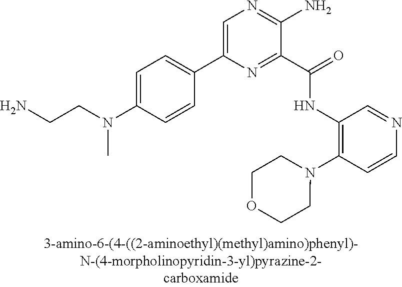 [SMILES: CN(CCN)c1ccc(-c2cnc(N)c(C(=O)Nc3cnccc3N3CCOCC3)n2)cc1] | ¹H NMR (400 MHz, Methanol-d₄) δ: 3.05 (s, 3H), 3.18-3.24 (m, 2H), 3.46-3.52 (m, 4H), 3.67-3.71 (m, 2H), 3.80-3.82 (m, 4H), 6.96 (d, J = 8.0 Hz, 2H), 7.51 (d, J = 4.0 Hz, 1H), 7.94 (d, J = 8.0 Hz, 2H), 8.36 (d, J = 4.0 Hz, 1H), 8.73 (s, 1H), 9.14 (s, 1H). |
| 273 | 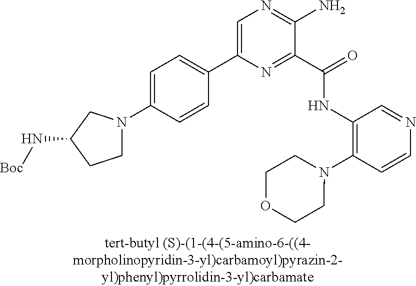 [SMILES: CC(C)(C)OC(=O)N[C@H]1CCN(c2ccc(-c3cnc(N)c(C(=O)Nc4cnccc4N4CCOCC4)n3)cc2)C1] | ¹H NMR (400 MHz, DMSO-d₆) δ: 1.47 (s, 9H), 1.91-1.94 (m, 1H), 2.16-2.20 (m, 1H), 2.95-2.98 (m, 4H), 3.10-3.14 (m, 1H), 3.33 (s, 1H), 3.42-3.53 (m, 2H), 3.74-3.76 (m, 4H), 4.17 (d, J = 6.0 Hz, 1H), 6.62 (d, J = 8.8 Hz, 2H), 7.24 (d, J = 5.6 Hz, 2H), 7.53 (s, 2H), 7.99 (d, J = 7.6 Hz, 2H), 8.31 (d, J = 5.2 Hz, 1H), 8.86 (s, 1H), 9.36 (s, 1H), 10.48 (s, 1H). |
| 274 | 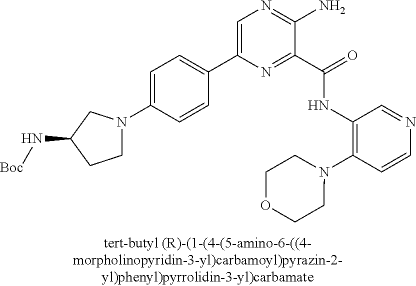 [SMILES: CC(C)(C)OC(=O)N[C@@H]1CCN(c2ccc(-c3cnc(N)c(C(=O)Nc4cnccc4N4CCOCC4)n3)cc2)C1] | ¹H NMR (400 MHz, Methanol-d₄) δ: 1.46 (s, 9H), 1.97-2.02 (m, 1H), 2.25-2.34 (m, 1H), 2.99-3.04 (m, 4H), 3.12-3.21 (m, 1H), 3.32-3.65 (m, 3H), 3.84-3.86 (m, 4H), 4.25-4.28 (m, 1H), 6.68 (d, J = 8.4 Hz, 2H), 7.26 (d, J = 5.6 Hz, 1H), 7.91 (d, J = 8.4 Hz, 2H), 8.27 (d, J = 5.2 Hz, 1H), 8.67 (s, 1H), 9.50 (s, 1H). |
| 275 | 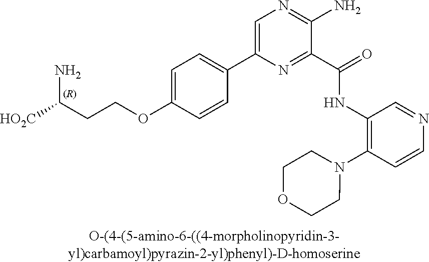 | ¹H NMR (300 MHz, Methanol-d₄) δ: 2.33- 2.40 (m, 1H), 2.48-2.53 (m, 1H), 3.38-3.40 (m, 4H), 3.75-3.83 (m, 4H), 4.02-4.08 (m, 1H), 4.25-4.32 (m, 2H), 7.14 (d, J = 8.7 Hz, 2H), 7.44 (d, J = 5.7 Hz, 1H), 8.02 (d, J = 8.7 Hz, 2H), 8.34 (d, J = 6.3 Hz, 1H), 8.77 (s, 1H), 9.22 (s, 1H). |
| 276 | 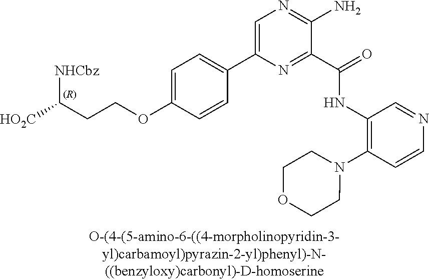 | ¹H NMR (400 MHz, DMSO-d₆) δ: 2.04- 2.33 (m, 2H), 2.86-2.96 (s, 4H), 3.61- 3.72 (s, 4H), 4.10-4.45 (m, 3H), 5.04 (s, 2H), 7.02 (d, J = 8.8 Hz, 2H), 7.23-7.24 (s, 1H), 7.28-7.35 (m, 5H), 7.67-7.72 (m, 3H), 8.09 (d, J = 8.8 Hz, 2H), 8.31 (d, J = 5.6 Hz, 1H), 8.92 (s, 1H), 9.33 (s, 1H), 10.44 (s, 1H), 12.70 (s, 1H). |
| 277 | 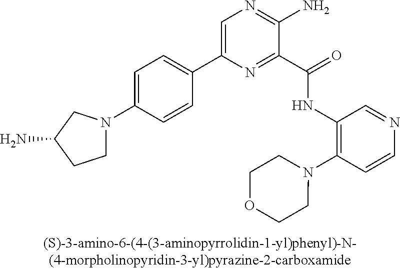 [SMILES: Nc1ncc(-c2ccc(N3CC[C@H](N)C3)cc2)nc1C(=O)Nc1cnccc1N1CCOCC1] | ¹H NMR (400 MHz, Methanol-d₄) δ: 1.91- 1.95 (m, 1H), 2.28-2.31 (m, 1H), 3.03-3.06 (m, 4H), 3.13-3.17 (m, 1H), 3.32-3.34 (m, 1H), 3.54-3.62 (m, 2H), 3.70-3.73 (m, 1H), 3.85-3.88 (m, 4H), 6.70 (d, J = 8.8 Hz, 2H), 7.27 (d, J = 5.2 Hz, 1H), 7.92 (d, J = 8.8 Hz, 2H), 8.28 (d, J = 5.2 Hz, 1H), 8.69 (s, 1H), 9.51 (s, 1H). |
| 278 | 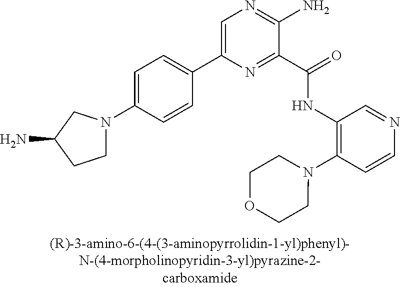 [SMILES: Nc1ncc(-c2ccc(N3CC[C@@H](N)C3)cc2)nc1C(=O)Nc1cnccc1N1CCOCC1] | ¹H NMR (400 MHz, Methanol-d₄) δ: 2.21- 2.27 (m, 1H), 2.49-2.57 (m, 1H), 3.46-3.66 (m, 6H), 3.68-3.73 (m, 2H), 3.83-3.85 (m, 4H), 4.07-4.12 (m, 1H), 6.81 (d, J = 8.8 Hz, 2H), 7.52 (d, J = 6.8 Hz, 1H), 7.97 (d, J = 8.8 Hz, 2H), 8.37 (d, J = 6.8 Hz, 1H), 8.75 (s, 1H), 9.18 (s, 1H). |
| 279 | 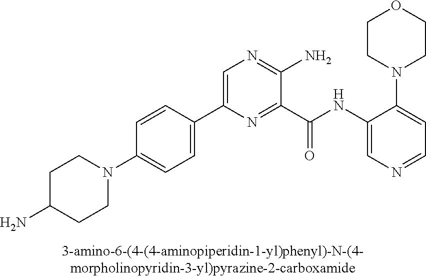 [SMILES: Nc1ncc(-c2ccc(N3CCC(N)CC3)cc2)nc1C(=O)Nc1cnccc1N1CCOCC1] | ¹H NMR (400 MHz, Methanol-d₄) δ: 1.54- 1.69 (m, 2H), 1.90-2.07 (m, 2H), 2.81-2.98 (m, 7H), 3.79-3.83 (m, 6H), 7.04 (d, J = 8.8 Hz, 2H), 7.23 (d, J = 5.2 Hz, 1H), 7.88 (d, J = 8.8 Hz, 2H), 8.27 (d, J = 5.2 Hz, 1H), 8.62 (s, 1H), 9.47 (s, 1H). |
| 280 | 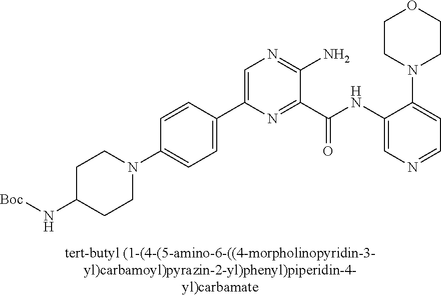 [SMILES: CC(C)(C)OC(=O)NC1CCN(c2ccc(-c3cnc(N)c(C(=O)Nc4cnccc4N4CCOCC4)n3)cc2)CC1] | ¹H NMR (400 MHz, DMSO-d₆) δ: 1.47- 1.54 (m, 11H), 1.80-1.82 (m, 2H), 2.80- 2.83 (m, 2H), 2.97 (s, 4H), 3.45 (s, 1H), 3.75 (s, 6H), 6.87 (s, 1H), 7.04 (d, J = 7.2 Hz, 2H), 7.24 (s, 1H), 7.60 (s, 2H), 8.00 (d, J = 7.6 Hz, 2H), 8.83 (s, 1H), 8.88 (s, 1H), 9.33 (s, 1H), 10.44 (s, 1H). |
| 281 | 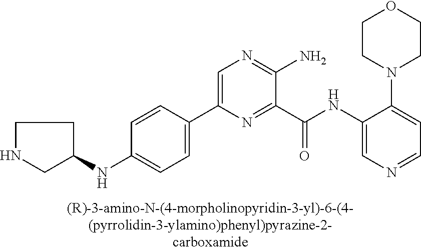 [SMILES: Nc1ncc(-c2ccc(N[C@@H]3CCNC3)cc2)nc1C(=O)Nc1cnccc1N1CCOCC1] | ¹H NMR (400 MHz, Methanol-d₄) δ: 2.05- 2.18 (m, 1H), 2.38-2.47 (m, 1H), 3.43-3.61 (m, 8H), 3.83-3.85 (m, 4H), 4.31-4.36 (m, 1H), 6.82 (d, J = 8.4 Hz, 2H), 7.54 (d, J = 6.8 Hz, 1H), 7.90 (d, J = 8.4 Hz, 2H), 8.37 (d, J = 6.8 Hz, 1H), 8.75 (s, 1H), 9.17 (s, 1H). |
| 282 | 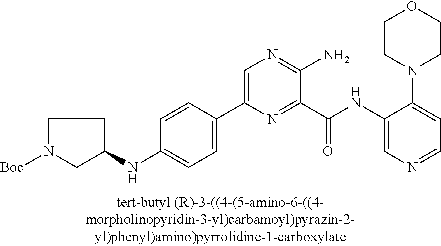 [SMILES: CC(C)(C)OC(=O)N1CC[C@@H](Nc2ccc(-c3cnc(N)c(C(=O)Nc4cnccc4N4CCOCC4)n3)cc2)C1] | ¹H NMR (400 MHz, Methanol-d₄) δ: 1.32- 1.35 (m, 1H), 1.48-4.50 (m, 9H), 1.96-1.99 (m, 1H), 2.25-2.28 (m, 1H), 3.05-3.07 (m, 4H), 3.15-3.16 (m, 1H), 3.21-3.29 (m, 2H), 3.48-3.57 (m, 2H), 3.66-3.73 (m, 1H), 3.86-3.88 (m, 4H), 4.14 (s, 1H), 6.80 (d, J = 8.4 Hz, 2H), 7.29-7.30 (m, 1H), 7.89 (d, J = 8.4 Hz, 2H), 8.30 (s, 2H), 8.69 (s, 1H), 9.55 (s, 1H). |
| 283 | 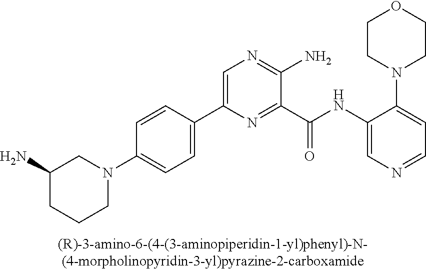 [SMILES: Nc1ncc(-c2ccc(N3CCC[C@@H](N)C3)cc2)nc1C(=O)Nc1cnccc1N1CCOCC1] | ¹H NMR (400 MHz, DMSO-d₆) δ: 1.17- 1.40 (m, 1H), 1.51-1.61 (m, 1H), 1.73- 1.87 (m, 2H), 2.61-2.80 (m, 2H), 2.95- 2.96 (m, 4H), 3.33 (s, 1H), 3.46-3.47 (m, 1H), 3.62-3.74 (m, 7H), 7.02 (d, J = 8.8 Hz, 2H), 7.23 (d, J = 5.2 Hz, 1H), 7.58 (s, 2H), 7.99 (d, J = 8.8 Hz, 2H), 8.31 (d, J = 5.2 Hz, 1H), 8.87 (s, 1H), 9.33 (s, 1H), 10.44 (s, 1H). |
| 284 | 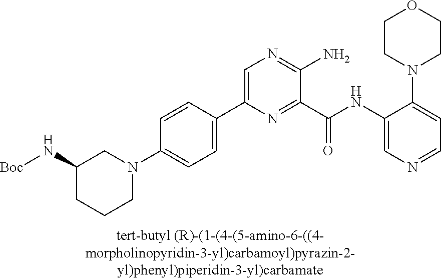 [SMILES: CC(C)(C)OC(=O)N[C@@H]1CCCN(c2ccc(-c3cnc(N)c(C(=O)Nc4cnccc4N4CCOCC4)n3)cc2)C1] | ¹H NMR (400 MHz, DMSO-d₆) δ: 1.54 (s, 10H), 1.57-1.60 (m, 1H), 1.74-1.86 (m, 2H), 2.63-2.80 (m, 2H), 2.96-2.98 (m, 4H), 3.30-3.47 (m, 1H), 3.73-3.74 (m, 6H), 6.89-6.91 (m, 1H), 7.04 (d, J = 8.4 Hz, 2H), 7.24 (d, J = 5.2 Hz, 1H), 7.58 (s, 2H), 8.00 (d, J = 8.4 Hz, 2H), 8.31 (d, J = 5.6 Hz, 1H), 8.88 (s, 1H), 9.33 (s, 1H), 10.45 (s, 1H). |
| 285 | 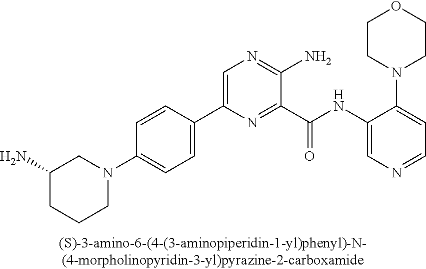 [SMILES: Nc1ncc(-c2ccc(N3CCC[C@H](N)C3)cc2)nc1C(=O)Nc1cnccc1N1CCOCC1] | ¹H NMR (400 MHz, Methanol-d₄) δ: 1.32- 1.41 (m, 1H), 1.68-1.71 (m, 1H), 1.74-1.76 (m, 1H), 1.86-2.01 (m, 1H), 2.66-2.71 (m, 1H), 2.84-3.02 (m, 6H), 3.36-3.59 (m, 1H), 3.61-3.70 (m, 1H), 3.73-3.84 (m, 4H), 7.07 (d, J = 8.8 Hz, 2H), 7.25 (d, J = 4.8 Hz, 1H), 7.91 (d, J = 8.8 Hz, 2H), 8.28 (s, 1H), 8.77 (s, 1H), 9.48 (s, 1H). |
| 286 | 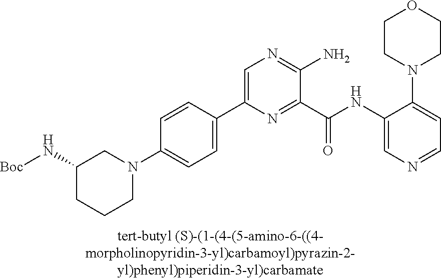 [SMILES: CC(C)(C)OC(=O)N[C@H]1CCCN(c2ccc(-c3cnc(N)c(C(=O)Nc4cnccc4N4CCOCC4)n3)cc2)C1] | ¹H NMR (400 MHz, DMSO-d₆) δ: 1.41 (s, 10H), 1.50-1.60 (m, 1H), 1.70-1.90 (m, 2H), 2.60-2.70 (m, 2H), 2.95-2.98 (m, 4H), 3.31- 3.50 (m, 1H), 3.73-3.75 (m, 6H), 6.80-6.90 (m, 1H), 7.03 (d, J = 8.8 Hz, 2H), 7.23 (d, J = 5.6 Hz, 1H), 7.50 (s, 2H), 8.00 (d, J = 8.8 Hz, 2H), 8.31 (d, J = 5.2 Hz, 1H), 8.87 (s, 1H), 9.32 (s, 1H), 10.45 (s, 1H). |
| 287 | 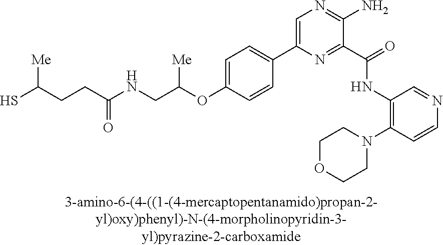 [SMILES: CC(S)CCC(=O)NCC(C)Oc1ccc(-c2cnc(N)c(C(=O)Nc3cnccc3N3CCOCC3)n2)cc1] | ¹H NMR (400 MHz, DMSO-d₆) δ: 1.20- 1.27 (m, 6H), 1.63-1.68 (m, 1H), 1.76- 1.81 (m, 1H), 2.08-2.27 (m, 2H), 2.39- 2.51 (m, 1H), 2.84-2.89 (m, 1H), 3.00- 3.19 (m, 4H), 3.20-3.24 (m, 1H), 3.40- 3.43 (m, 1H), 3.72-3.74 (m, 4H), 4.54- 4.58 (m, 1H), 7.12 (d, J = 8.8 Hz, 2H), 7.24 (d, J = 5.2 Hz, 1H), 7.66 (s, 2H), 8.08-8.14 (m, 3H), 8.32 (d, J = 5.2 Hz, 1H), 8.92 (s, 1H), 9.34 (s, 1H), 10.45 (s, 1H). |
| 288 | 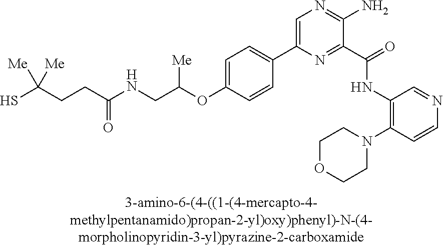 [SMILES: CC(CNC(=O)CCC(C)(C)S)Oc1ccc(-c2cnc(N)c(C(=O)Nc3cnccc3N3CCOCC3)n2)cc1] | ¹H NMR (400 MHz, DMSO-d₆) δ: 1.27- 1.29 (m, 9H), 1.75-1.79 (m, 2H), 2.24- 2.28 (m, 2H), 2.58 (s, 1H), 2.98-2.99 (m, 4H), 3.34-3.37 (m, 1H), 3.40-3.42 (m, 1H), 3.72-3.74 (m, 4H), 4.30-4.40 (m, 1H), 7.12 (d, J = 8.8 Hz, 2H), 7.24 (d, J = 5.2 Hz, 1H), 7.70 (s, 2H), 8.08- 8.10 (m, 3H), 8.32 (d, J = 5.2 Hz, 1H), 8.92 (s, 1H), 9.34 (s, 1H), 10.46 (s, 1H). |
| 289 | 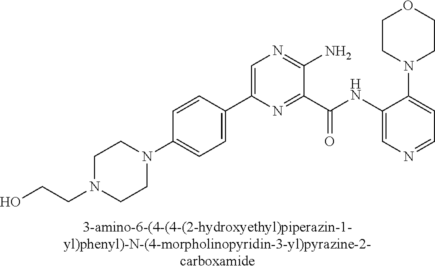 [SMILES: Nc1ncc(-c2ccc(N3CCN(CCO)CC3)cc2)nc1C(=O)Nc1cnccc1N1CCOCC1] | ¹H NMR (400 MHz, Methanol-d₄) δ: 2.60- 2.67 (m, 2H), 2.72-2.74 (m, 4H), 3.03-3.18 (m, 5H), 3.30-3.31 (m, 3H), 3.73-3.76 (m, 2H), 3.83-3.85 (m, 4H), 7.10 (d, J = 8.8 Hz, 2H), 7.20-7.27 (m, 1H), 7.93-7.98 (m, 2H), 8.24-8.28 (m, 1H), 8.72 (s, 1H), 9.38 (s, 1H). |
| 290 | 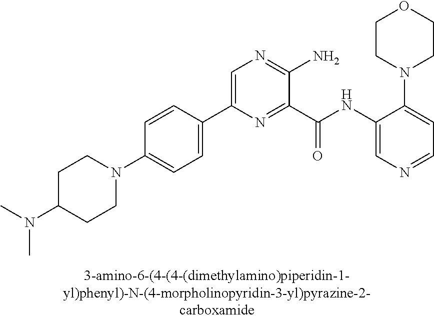 [SMILES: CN(C)C1CCN(c2ccc(-c3cnc(N)c(C(=O)Nc4cnccc4N4CCOCC4)n3)cc2)CC1] | ¹H NMR (400 MHz, DMSO-d₆) δ: 1.50- 1.51 (m, 2H), 1.84-1.87 (m, 2H), 2.07- 2.21 (m, 7H), 2.74-2.79 (m, 2H), 2.96- 2.98 (m, 4H), 3.73-3.75 (m, 4H), 3.81- 3.84 (m, 2H), 7.05 (d, J = 8.8 Hz, 2H), 7.24 (d, J = 5.2 Hz, 1H), 7.58 (s, 2H), 8.00 (d, J = 8.8 Hz, 2H), 8.31 (d, J = 5.6 Hz, 1H), 8.88 (s, 1H), 9.34 (s, 1H), 10.45 (s, 1H). |
| 291 | 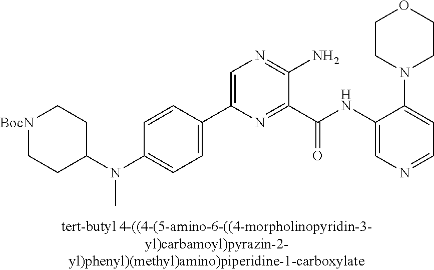 [SMILES: CN(c1ccc(-c2cnc(N)c(C(=O)Nc3cnccc3N3CCOCC3)n2)cc1)C1CCN(C(=O)OC(C)(C)C)CC1] | ¹H NMR (400 MHz, DMSO-d₆) δ: 1.54 (s, 9H), 1.56-1.67 (m, 4H), 2.78-2.96 (m, 9H), 3.74 (s, 4H), 3.90-3.96 (m, 1H), 4.05-4.08 (m, 2H), 6.93 (d, J = 8.8 Hz, 2H), 7.25 (s, 1H), 7.55 (s, 2H), 7.97 (d, J = 8.8 Hz, 2H), 8.30 (s, 1H), 8.86 (s, 1H), 9.30 (s, 1H), 10.48 (s, 1H). |
| 292 | 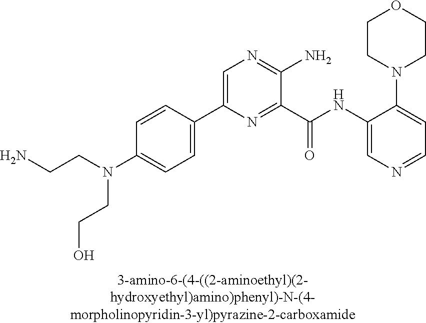 [SMILES: NCCN(CCO)c1ccc(-c2cnc(N)c(C(=O)Nc3cnccc3N3CCOCC3)n2)cc1] | ¹H NMR (400 MHz, Methanol-d₄) δ: 2.94- 2.96 (m, 2H), 3.04-3.09 (m, 5H), 3.43-3.46 (m, 2H), 3.74-3.77 (m, 2H), 3.86-3.88 (m, 3H), 4.82 (s, 2H), 6.82 (d, J = 8.8 Hz, 2H), 7.28 (d, J = 5.6 Hz, 1H), 7.90 (d, J = 8.8 Hz, 2H), 8.29 (d, J = 5.2 Hz, 1H), 8.69 (s, 1H), 9.51 (s, 1H). |
| 293 | 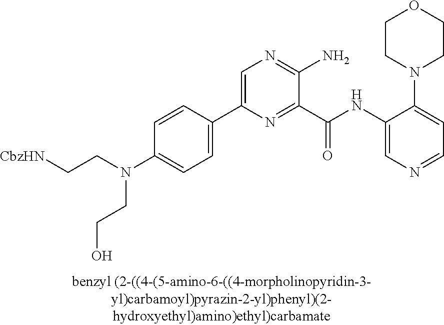 [SMILES: Nc1ncc(-c2ccc(N(CCO)CCNC(=O)OCc3ccccc3)cc2)nc1C(=O)Nc1cnccc1N1CCOCC1] | ¹H NMR (400 MHz, DMSO-d₆) δ: 2.55- 2.58 (m, 2H), 2.69-2.71 (m, 2H), 2.97- 3.00 (m, 4H), 3.52-3.54 (m, 3H), 3.71- 3.81 (m, 6H), 4.48 (br, 1H), 5.13 (s, 2H), 7.24 (d, J = 5.2 Hz, 1H), 7.30-7.34 (m, 5H), 7.56 (d, J = 8.4 Hz, 2H), 7.71 (br, 2H), 8.16 (d, J = 8.4 Hz, 2H), 8.33 (d, J = 5.2 Hz, 1H), 8.99 (s, 1H), 9.30 (s, 1H), 10.45 (s, 1H). |
| 294 | 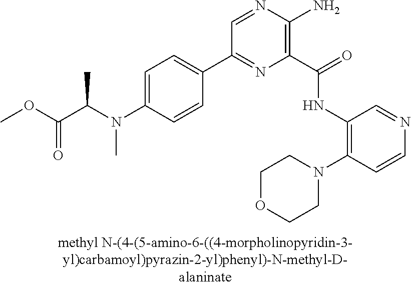 [SMILES: COC(=O)[C@@H](C)N(C)c1ccc(-c2cnc(N)c(C(=O)Nc3cnccc3N3CCOCC3)n2)cc1] | ¹H NMR (400 MHz, DMSO-d₆) δ: 1.40- 1.44 (m, 3H), 2.87 (s, 3H), 2.90-3.01 (m, 4H), 3.65 (s, 3H), 3.71-3.80 (m, 4H), 4.76-4.83 (m, 1H), 6.89 (d, J = 8.8 Hz, 2H), 7.21-7.29 (m, 1H), 7.58 (s, 2H), 7.99 (d, J = 8.8 Hz, 2H), 8.31 (s, 1H), 8.87 (s, 1H), 9.33 (s, 1H), 10.45 (s, 1H). |
| 295 | 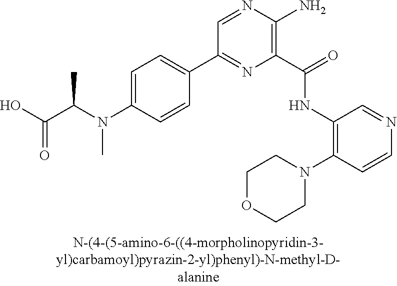 [SMILES: C[C@H](C(=O)O)N(C)c1ccc(-c2cnc(N)c(C(=O)Nc3cnccc3N3CCOCC3)n2)cc1] | ¹H NMR (400 MHz, Methanol-d₄) δ: 1.41- 1.53 (m, 3H), 2.94-3.08 (m, 7H), 3.79- 3.87 (m, 4H), 4.36-4.45 (m, 1H), 6.87- 6.93 (m, 2H), 7.20-7.23 (m, 1H), 7.79- 7.90 (m, 2H), 8.25 (s, 1H), 8.60-8.63 (m, 1H), 9.43 (s, 1H). |
| 296 | 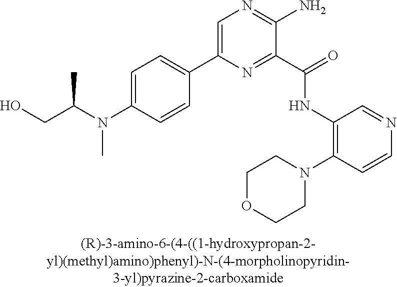 [SMILES: C[C@H](CO)N(C)c1ccc(-c2cnc(N)c(C(=O)Nc3cnccc3N3CCOCC3)n2)cc1] | ¹H NMR (400 MHz, DMSO-d₆) δ: 1.11 (d, J = 6.8 Hz, 3H), 2.79 (s, 3H), 2.97 (s, 4H), 3.43-3.59 (m, 2H), 3.76 (s, 4H), 4.00-4.05 (m, 1H), 4.70-4.72 (m, 1H), 6.88 (d, J = 8.8 Hz, 2H), 7.24 (d, J = 5.2 Hz, 1H), 7.54 (s, 2H), 7.97 (d, J = 8.4 Hz, 2H), 8.32 (d, J = 5.2 Hz, 1H), 8.86 (s, 1H), 9.35 (s, 1H), 10.46 (s, 1H). |
| 297 | 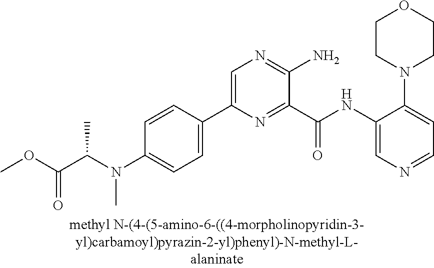 [SMILES: COC(=O)[C@H](C)N(C)c1ccc(-c2cnc(N)c(C(=O)Nc3cnccc3N3CCOCC3)n2)cc1] | ¹H NMR (400 MHz, DMSO-d₆) δ: 1.40- 1.50 (m, 3H), 2.90 (s, 3H), 2.97-2.99 (m, 4H), 3.65 (s, 3H), 3.74-3.76 (m, 4H), 4.78-4.83 (m, 1H), 6.90 (d, J = 8.8 Hz, 2H), 7.25 (d, J = 4.8 Hz, 1H), 7.60 (s, 2H), 8.00 (d, J = 8.8 Hz, 2H), 8.33 (s, 1H), 8.90 (s, 1H), 9.34 (s, 1H), 10.47 (s, 1H). |
| 298 | 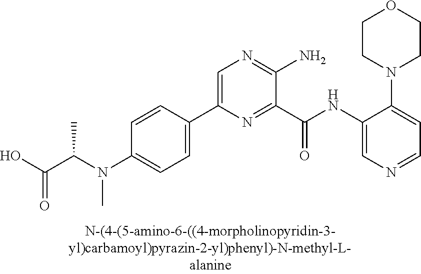 [SMILES: C[C@@H](C(=O)O)N(C)c1ccc(-c2cnc(N)c(C(=O)Nc3cnccc3N3CCOCC3)n2)cc1] | ¹H NMR (400 MHz, DMSO-d₆ + D₂O) δ: 1.28 (d, J = 7.2 Hz, 3H), 2.84- 2.90 (m, 7H), 3.71 (s, 5H), 6.76 (d, J = 8.8 Hz, 2H), 7.21 (d, J = 5.6 Hz, 1H), 7.85 (d, J = 8.4 Hz, 2H), 8.23 (d, J = 4.8 Hz, 1H), 8.69 (s, 1H), 9.18 (s, 1H). |
| 299 | 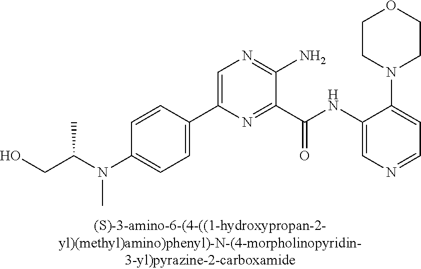 [SMILES: C[C@@H](CO)N(C)c1ccc(-c2cnc(N)c(C(=O)Nc3cnccc3N3CCOCC3)n2)cc1] | ¹H NMR (400 MHz, DMSO-d₆) δ: 1.11 (d, J = 6.8 Hz, 3H), 2.79 (s, 3H), 2.98- 2.99 (m, 4H), 3.40-3.60 (m, 2H), 3.75- 3.78 (m, 4H), 3.90-4.10 (m, 1H), 4.70- 4.75 (m, 1H), 6.88 (d, J = 9.2 Hz, 2H), 7.20-7.30 (m, 1H), 7.50 (s, 2H), 7.98 (d, J = 8.4 Hz, 2H), 8.32 (d, J = 5.2 Hz, 1H), 8.87 (s, 1H), 9.35 (s, 1H), 10.47 (s, 1H). |
| 300 | 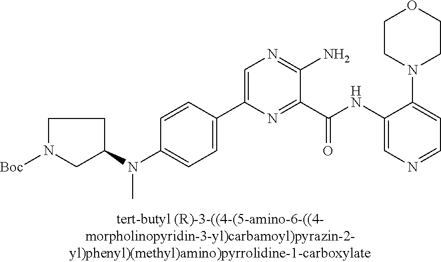 [SMILES: CN(c1ccc(-c2cnc(N)c(C(=O)Nc3cnccc3N3CCOCC3)n2)cc1)[C@@H]1CCN(C(=O)OC(C)(C)C)C1] | ¹H NMR (400 MHz, Methanol-d₄) δ: 1.50 (s, 9H), 2.13-2.16 (m, 2H), 2.90 (s, 3H), 2.97-3.00 (m, 4H), 3.36-3.41 (m, 2H), 3.52-3.65 (m, 2H), 3.80-3.82 (m, 4H), 4.53-4.57 (m, 1H), 6.97 (d, J = 8.8 Hz, 2H), 7.23-7.24 (m, 1H), 7.89 (d, J = 8.4 Hz, 2H), 8.26 (s, 1H), 8.64 (s, 1H), 9.47 (s, 1H). |
| 301 | 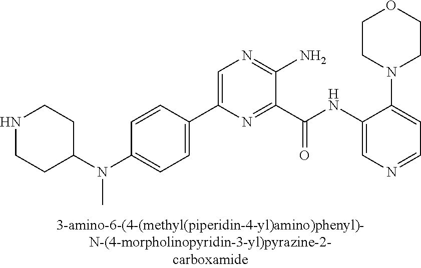 [SMILES: CN(c1ccc(-c2cnc(N)c(C(=O)Nc3cnccc3N3CCOCC3)n2)cc1)C1CCNCC1] | ¹H NMR (400 MHz, Methanol-d₄) δ: 2.04- 2.10 (m, 4H), 2.94 (s, 3H), 3.13-3.19 (m, 2H), 3.52-3.59 (m, 6H), 3.81-3.83 (m, 4H), 4.12-4.17 (m, 1H), 7.11 (d, J = 8.8 Hz, 2H), 7.51-7.53 (m, 1H), 7.97 (d, J = 8.8 Hz, 2H), 8.35 (s, 1H), 8.78 (s, 1H), 9.12 (s, 1H). |

### Example 6: Preparation of Amino-pyrazinecarboxamide-Linker Compounds Example 6.1: Preparation of 4-((S)-2-((S)-2-(6-(2,5-Dioxo-2,5-dihydro-1H-pyrrol-1-yl)hexanamido)-3-methylbutanamido)-5-ureidopentanamido)benzyl (2-(4-(5-amino-6-((4-morpholinopyridin-3-yl)carbamoyl)pyrazin-2-yl)phenyl)ethyl)carbamate (Compound 6.1)

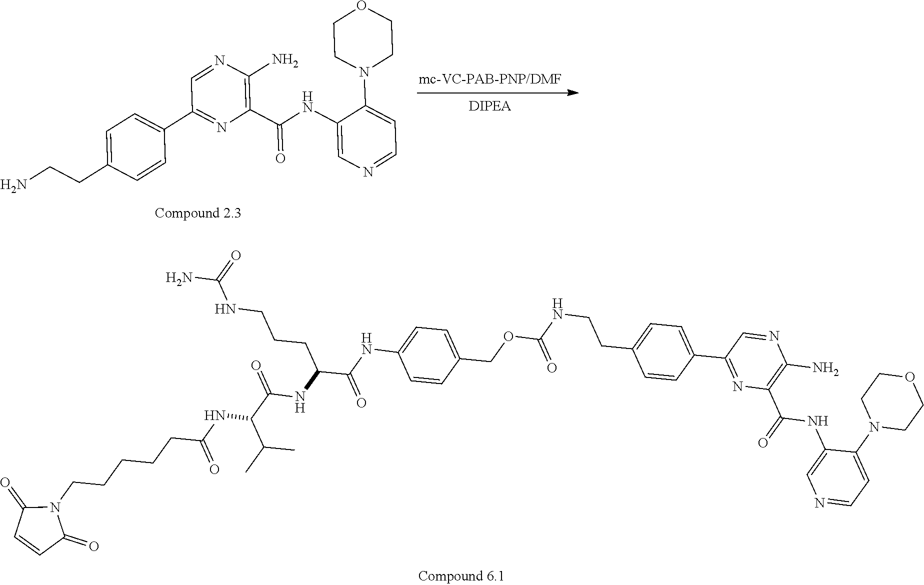 [SMILES: NCCc1ccc(-c2cnc(N)c(C(=O)Nc3cnccc3N3CCOCC3)n2)cc1 ; CC(C)[C@H](NC(=O)CCCCCN1C(=O)C=CC1=O)C(=O)N[C@@H](CCCNC(N)=O)C(=O)Nc1ccc(COC(=O)NCCc2ccc(-c3cnc(N)c(C(=O)Nc4cnccc4N4CCOCC4)n3)cc2)cc1]

A solution containing 42.0 mg (0.10 mmol) of Compound 2.3 was dissolved in 2 mL of DMF then treated with 74 mg (0.10 mmol) of mc-Val-Cit-PAB-PNP and 0.035 mL (0.2 mmol) of Hunig's base. The reaction was heated at 25° C. for 1 h then purified without work-up using RP-HPLC. Product fractions were identified by LCMS and pooled then lyophilized to provide 20 mg of the TFA salt of Compound 6.1 as a white solid. ¹H NMR (CD₃OD) δ 9.49 (s, 1H), 8.76 (s, 1H), 8.27 (d, J=5.2 Hz, 1H), 7.98 (J=8.0 Hz, 2H), 7.53 (d, J=8.4 Hz, 2H), 7.37 (d, J=7.6 Hz, 1H), 7.27 (m, 3H), 6.77 (s, 2H), 5.02 (s, 2H), 4.51-4.48 (m, 1H), 4.15 (d, J=7.6 Hz, 1H), 3.79 (m, 4H), 3.46-3.39 (m, 4H), 3.02 (m, 4H), 3.87 (t, J=6.8 Hz, 2H), 2.26 (t, J=7.2 Hz, 2H), 2.08-2.03 (m, 1H), 1.90-1.69 (m, 2H), 1.65-1.51 (m, 6H), 1.33-1.29 (m, 4H), 0.95 (d, J=6.8 Hz, 6H). LCMS (M+H)=1018.7.

The linker-compounds in Tables 15 and 16 were prepared in a manner similar to that described in Example 6.1.

TABLE 15

| Cmpd- | | |
|---|---|---|
| Linker | Structure/Name | ¹H NMR/MS |
| LP1 | 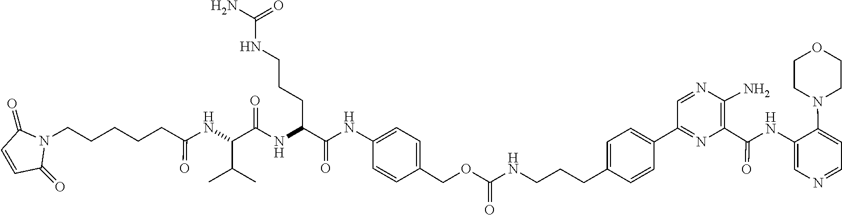 4-((S)-2-((S)-2-(6-(2,5-Dioxo-2,5-dihydro-1H-pyrrol- 1-yl)hexanamido)-3-methylbutanamido)-5- ureidopentanamido)benzyl (3-(4-(5-amino-6-((4- morpholinopyridin-3-yl)carbamoyl)pyrazin-2- yl)phenyl)propyl)carbamate | (DMSO-d⁶) δ 11.1 (s, 1H), 10.5 (s, 1H), 9.98 (s, 1H), 8.97 (s, 1H), 8.77 (s, 1H), 8.38 (dd, J = 0.8, 6.8 Hz, 1H), 8.15-8.07 (m, 3H), 7.85 (t, J = 6.4 Hz, 1H), 7.68 (bs, 1H), 7.59 (d, J = 8.4 Hz, 2H), 7.41 (d, J = 6.8 Hz, 1H), 7.37 (d, J = 8.4 Hz, 2H), 7.30 (d, J = 8.4 Hz, 2H), 7.00 (s, 2H), 6.93 (m, 1H), 5.98 (m, 1H), 5.41 (bs, 1H), 4.94 (s, 2H), 4.44-4.35 (m, 1H), 4.26 (t, J = 7.2 Hz, 2H), 3.69 (m, 6H), 3.56-3.50 (m, 6H), 3.36 (t, J = 6.8 Hz, 2H), 3.07-3.00 (m, 4H), 2.67 (m, 2H), 2.22-1.90 (m, 3H), 1.70- 1.29 (m, 12H), 1.22 (m, 2H), 0.85 (d, J = 6.8 Hz, 6H). [M + H]⁺: 1032.5 |
| LP9 | 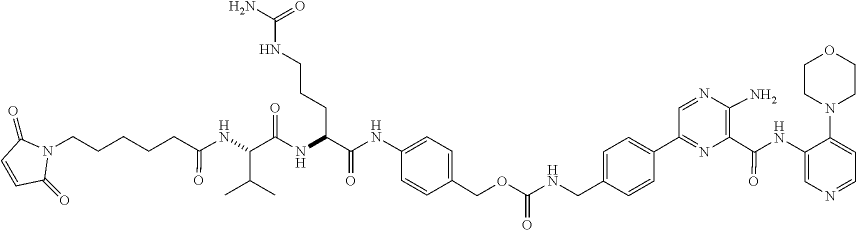 4-((S)-2-((S)-2-(6-(2,5-dioxo-2,5-dihydro-1H-pyrrol- 1-yl)hexanamido)-3-methylbutanamido)-5- ureidopentanamido)benzyl (4-(5-amino-6-((4- morpholinopyridin-3-yl)carbamoyl)pyrazin-2- yl)benzyl)carbamate | (DMSO-d⁶) δ 10.5 (s, 1H), 9.98 (s, 1H), 8.97 (s, 1H), 8.77 (s, 1H), 8.38 (dd, J = 0.8, 6.8 Hz, 1H), 8.16 (J = 8.4 Hz, 2H), 8.07 (d, J = 7.6 Hz, 1H), 7.85 (t, J = 6.4 Hz, 1H), 7.80 (d, J = 8.4 Hz, 1H), 7.68 (bs, 1H), 7.59 (d, J = 8.4 Hz, 2H), 7.41 (d, J = 6.8 Hz, 1H), 7.37 (d, J = 8.4 Hz, 2H), 7.30 (d, J = 8.4 Hz, 2H), 7.00 (s, 2H), 5.98 (s, 1H), 5.41 (bs, 1H), 4.98 (s, 2H), 4.44-4.40 (m, 1H), 4.26 (d, J = 6.4 Hz, 2H), 4.18 (t, J = 7.2 Hz, 1H), 3.79 (m, 6H), 3.56-3.50 (m, 6H), 3.36 (t, J = 6.8 Hz, 2H), 3.07-3.00 (m, 2H), 2.22-1.90 (m, 3H), 1.70-1.29 (m, 7H), 1.22 (m, 2H), 0.85 (d, J = 6.8 Hz, 6H). [M + H]⁺: 1004.5 |

TABLE 16

| Com- | | MS |
|---|---|---|
| pound- | | [M + |
| Linker | Structure | H]⁺ |
| LP10 | 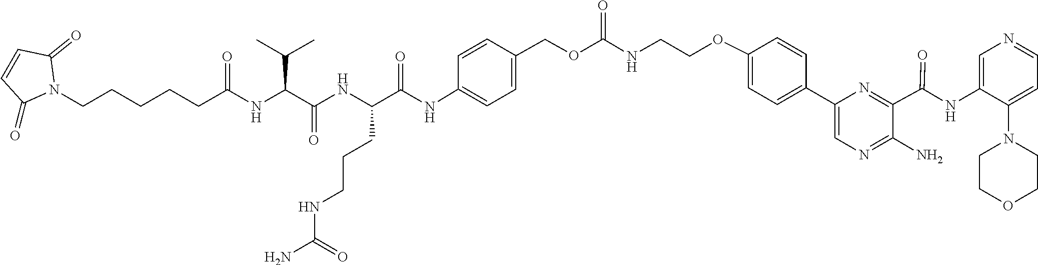 [SMILES: CC(C)[C@H](NC(=O)CCCCCN1C(=O)C=CC1=O)C(=O)N[C@@H](CCCNC(N)=O)C(=O)Nc1ccc(COC(=O)NCCOc2ccc(-c3cnc(N)c(C(=O)Nc4cnccc4N4CCOCC4)n3)cc2)cc1] | 627.4 |
| LP11 | 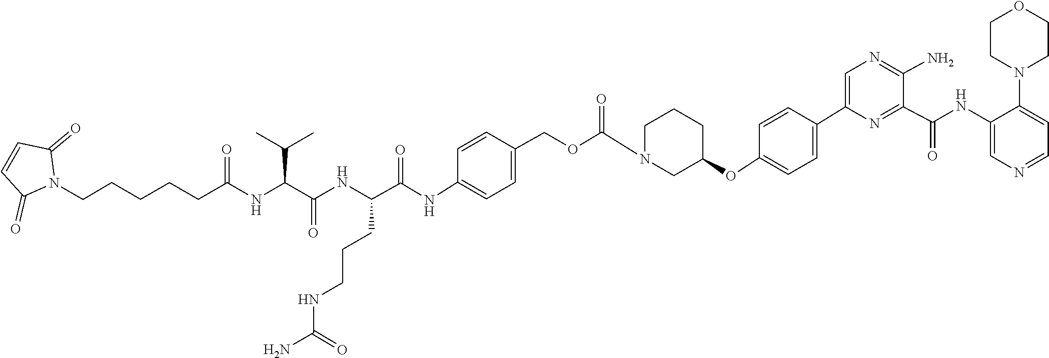 [SMILES: CC(C)[C@H](NC(=O)CCCCCN1C(=O)C=CC1=O)C(=O)N[C@@H](CCCNC(N)=O)C(=O)Nc1ccc(COC(=O)N2CCC[C@@H](Oc3ccc(-c4cnc(N)c(C(=O)Nc5cnccc5N5CCOCC5)n4)cc3)C2)cc1] | 766.5 |
| LP12 | 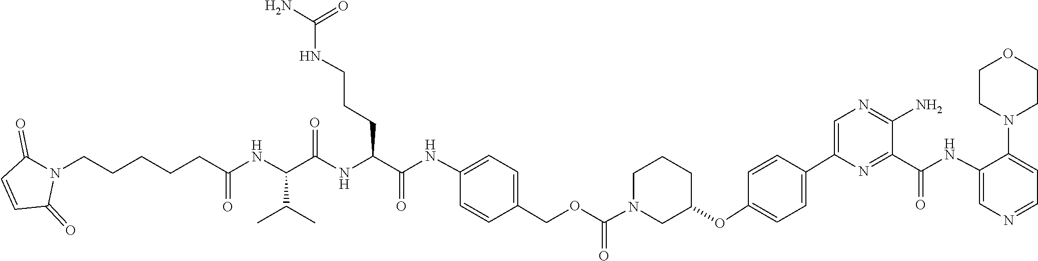 [SMILES: CC(C)[C@H](NC(=O)CCCCCN1C(=O)C=CC1=O)C(=O)N[C@@H](CCCNC(N)=O)C(=O)Nc1ccc(COC(=O)N2CCC[C@H](Oc3ccc(-c4cnc(N)c(C(=O)Nc5cnccc5N5CCOCC5)n4)cc3)C2)cc1] | 1074.6 |
| LP13 | 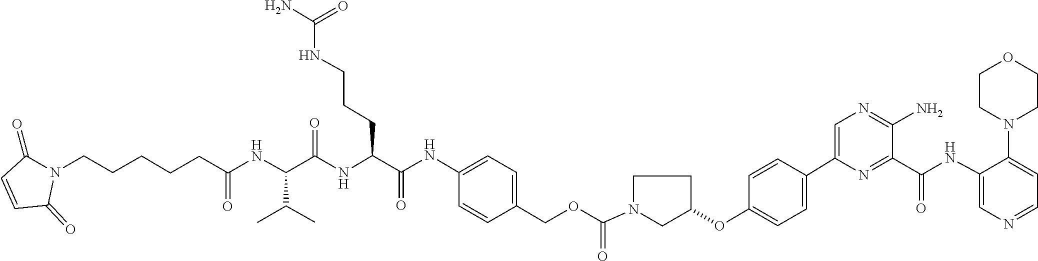 [SMILES: CC(C)[C@H](NC(=O)CCCCCN1C(=O)C=CC1=O)C(=O)N[C@@H](CCCNC(N)=O)C(=O)Nc1ccc(COC(=O)N2CC[C@H](Oc3ccc(-c4cnc(N)c(C(=O)Nc5cnccc5N5CCOCC5)n4)cc3)C2)cc1] | 1060.5 |
| LP14 | 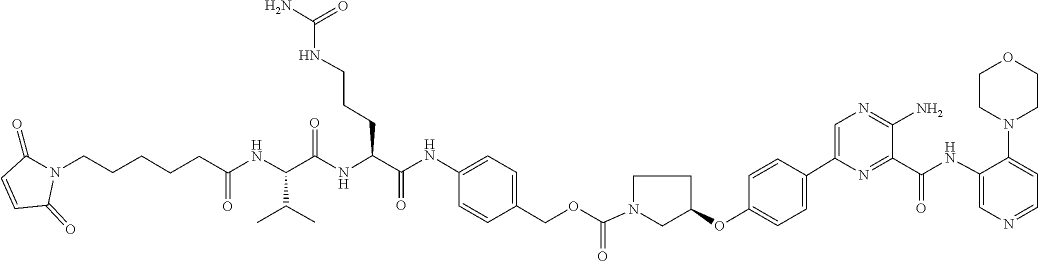 [SMILES: CC(C)[C@H](NC(=O)CCCCCN1C(=O)C=CC1=O)C(=O)N[C@@H](CCCNC(N)=O)C(=O)Nc1ccc(COC(=O)N2CC[C@@H](Oc3ccc(-c4cnc(N)c(C(=O)Nc5cnccc5N5CCOCC5)n4)cc3)C2)cc1] | 1060.5 |
| LP15 | 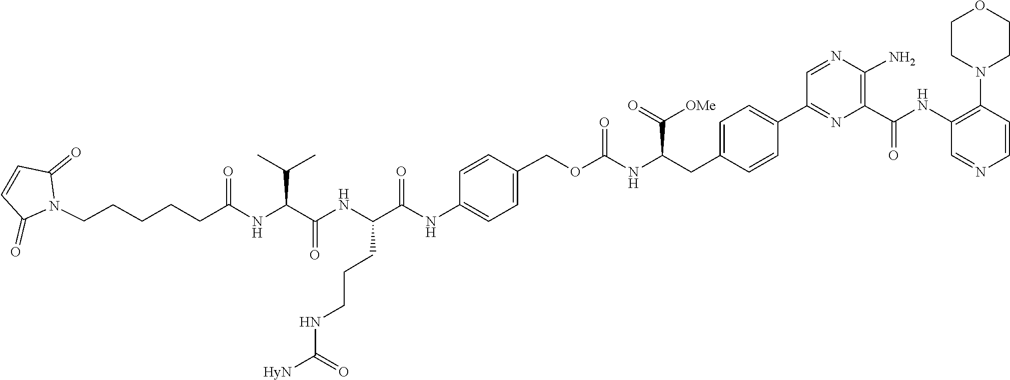 | 1076.6 |
| LP16 | 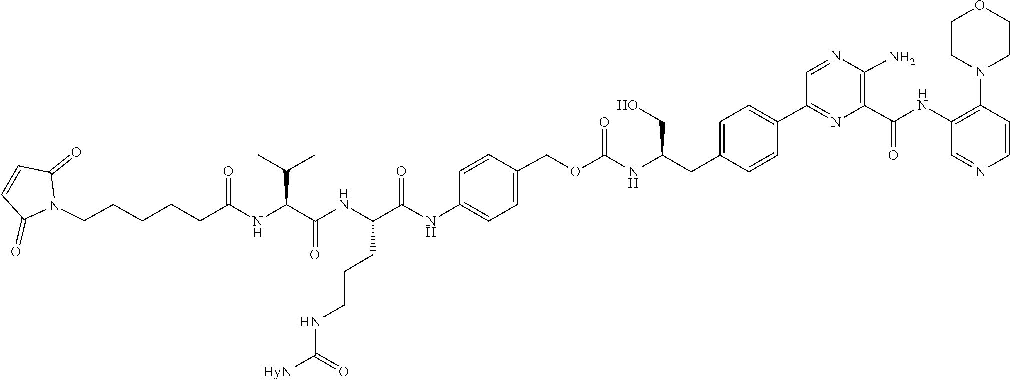 | 1048.6 |
| LP17 | 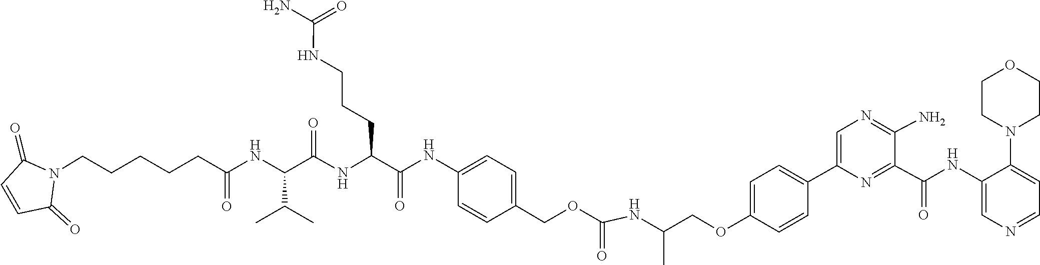 [SMILES: CC(COc1ccc(-c2cnc(N)c(C(=O)Nc3cnccc3N3CCOCC3)n2)cc1)NC(=O)OCc1ccc(NC(=O)[C@H](CCCNC(N)=O)NC(=O)[C@@H](NC(=O)CCCCCN2C(=O)C=CC2=O)C(C)C)cc1] | 1048.6 |
| LP18 | 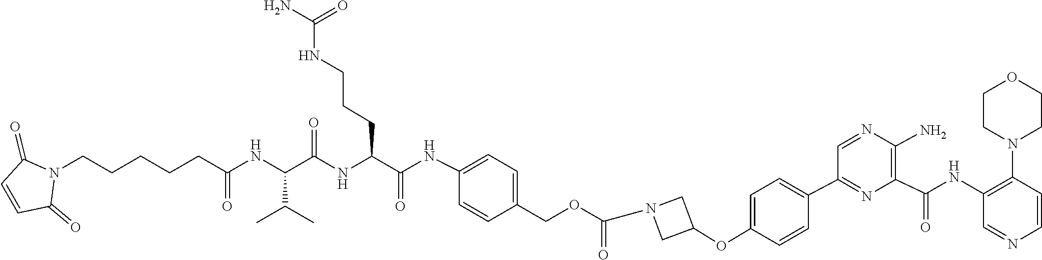 [SMILES: CC(C)[C@H](NC(=O)CCCCCN1C(=O)C=CC1=O)C(=O)N[C@@H](CCCNC(N)=O)C(=O)Nc1ccc(COC(=O)N2CC(Oc3ccc(-c4cnc(N)c(C(=O)Nc5cnccc5N5CCOCC5)n4)cc3)C2)cc1] | 1046.6 |
| LP19 | 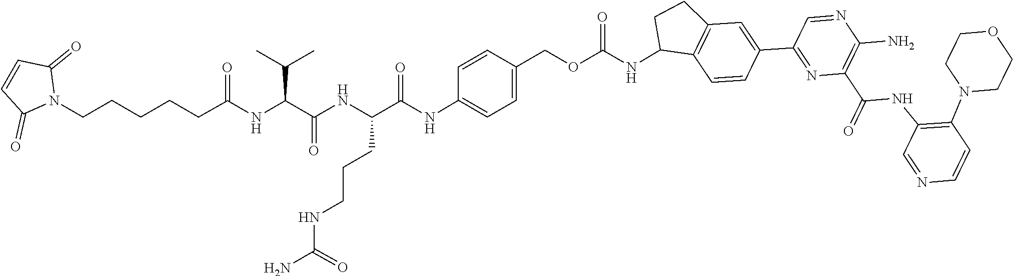 [SMILES: CC(C)[C@H](NC(=O)CCCCCN1C(=O)C=CC1=O)C(=O)N[C@@H](CCCNC(N)=O)C(=O)Nc1ccc(COC(=O)NC2CCc3cc(-c4cnc(N)c(C(=O)Nc5cnccc5N5CCOCC5)n4)ccc32)cc1] | 1030.6 |
| LP20 | 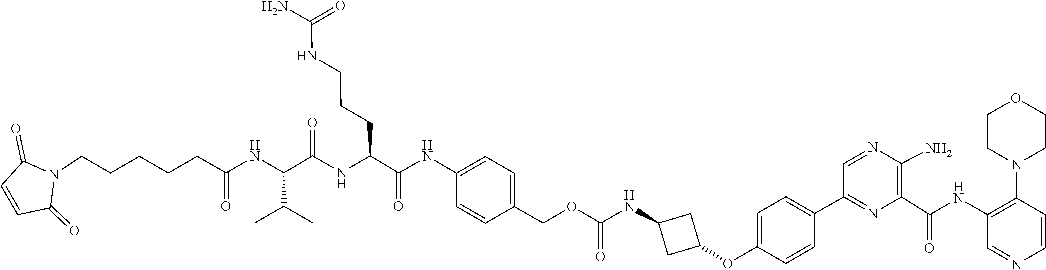 [SMILES: CC(C)[C@H](NC(=O)CCCCCN1C(=O)C=CC1=O)C(=O)N[C@@H](CCCNC(N)=O)C(=O)Nc1ccc(COC(=O)N[C@H]2C[C@H](Oc3ccc(-c4cnc(N)c(C(=O)Nc5cnccc5N5CCOCC5)n4)cc3)C2)cc1] | 1060.6 |
| LP21 | 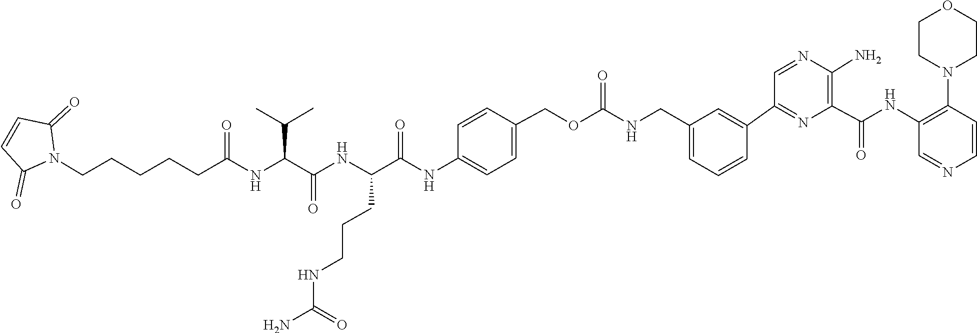 [SMILES: CC(C)[C@H](NC(=O)CCCCCN1C(=O)C=CC1=O)C(=O)N[C@@H](CCCNC(N)=O)C(=O)Nc1ccc(COC(=O)NCc2cccc(-c3cnc(N)c(C(=O)Nc4cnccc4N4CCOCC4)n3)c2)cc1] | 1004.6 |
| LP22 | 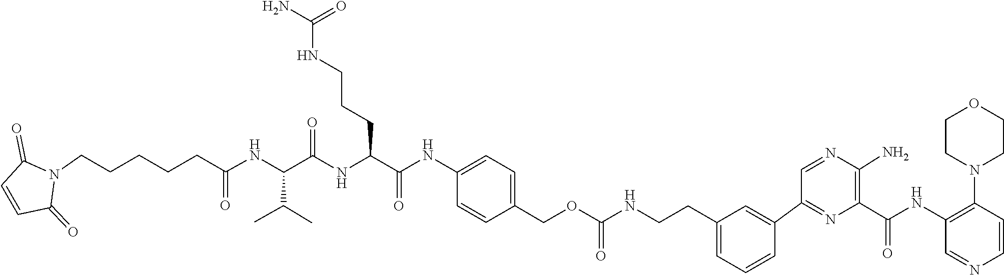 [SMILES: CC(C)[C@H](NC(=O)CCCCCN1C(=O)C=CC1=O)C(=O)N[C@@H](CCCNC(N)=O)C(=O)Nc1ccc(COC(=O)NCCc2cccc(-c3cnc(N)c(C(=O)Nc4cnccc4N4CCOCC4)n3)c2)cc1] | 1018.6 |
| LP23 | 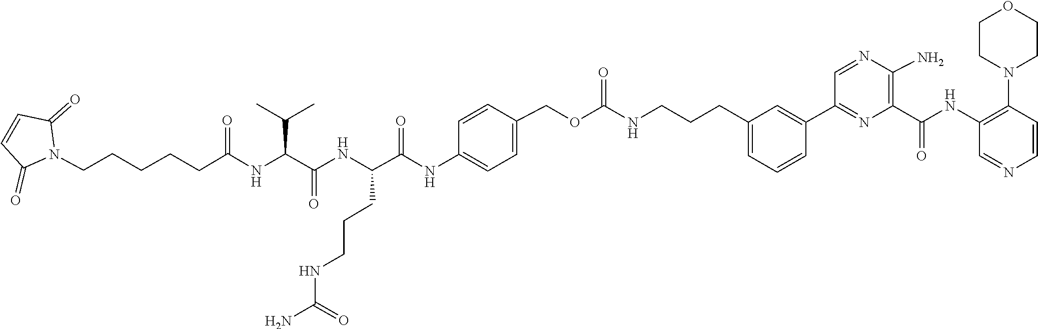 [SMILES: CC(C)[C@H](NC(=O)CCCCCN1C(=O)C=CC1=O)C(=O)N[C@@H](CCCNC(N)=O)C(=O)Nc1ccc(COC(=O)NCCCc2cccc(-c3cnc(N)c(C(=O)Nc4cnccc4N4CCOCC4)n3)c2)cc1] | 1032.6 |
| LP24 | 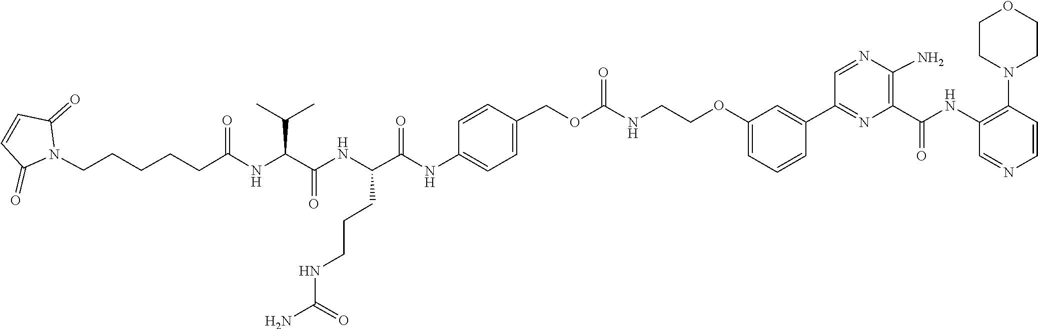 [SMILES: CC(C)[C@H](NC(=O)CCCCCN1C(=O)C=CC1=O)C(=O)N[C@@H](CCCNC(N)=O)C(=O)Nc1ccc(COC(=O)NCCOc2cccc(-c3cnc(N)c(C(=O)Nc4cnccc4N4CCOCC4)n3)c2)cc1] | 1034.6 |
| LP25 | 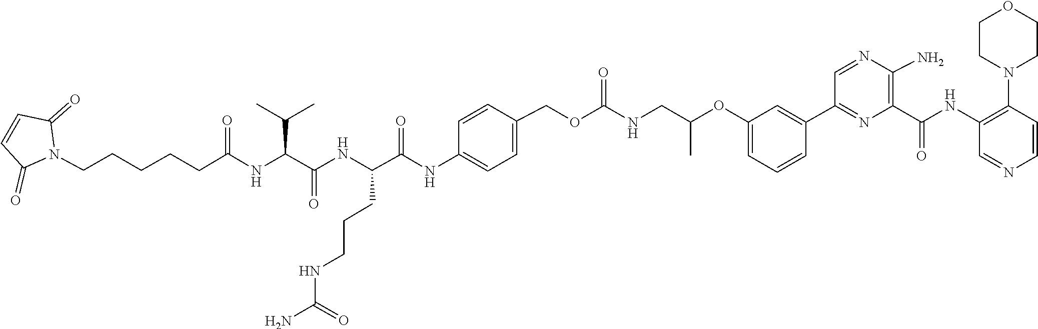 [SMILES: CC(CNC(=O)OCc1ccc(NC(=O)[C@H](CCCNC(N)=O)NC(=O)[C@@H](NC(=O)CCCCCN2C(=O)C=CC2=O)C(C)C)cc1)Oc1cccc(-c2cnc(N)c(C(=O)Nc3cnccc3N3CCOCC3)n2)c1] | 1048.6 |
| LP26 | 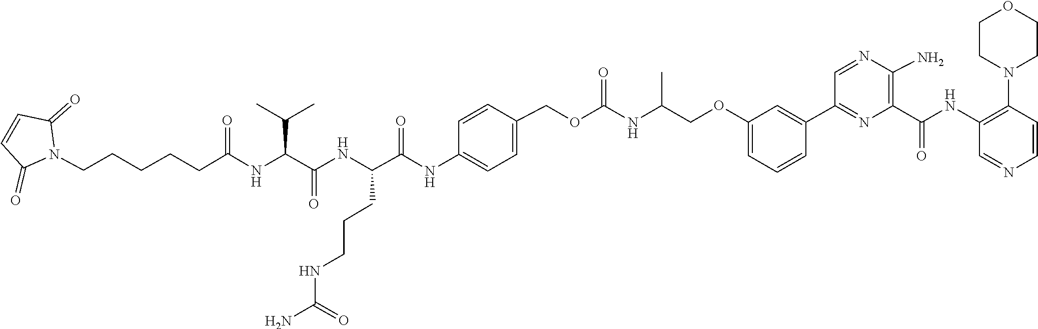 [SMILES: CC(COc1cccc(-c2cnc(N)c(C(=O)Nc3cnccc3N3CCOCC3)n2)c1)NC(=O)OCc1ccc(NC(=O)[C@H](CCCNC(N)=O)NC(=O)[C@@H](NC(=O)CCCCCN2C(=O)C=CC2=O)C(C)C)cc1] | 1048.6 |
| LP27 | 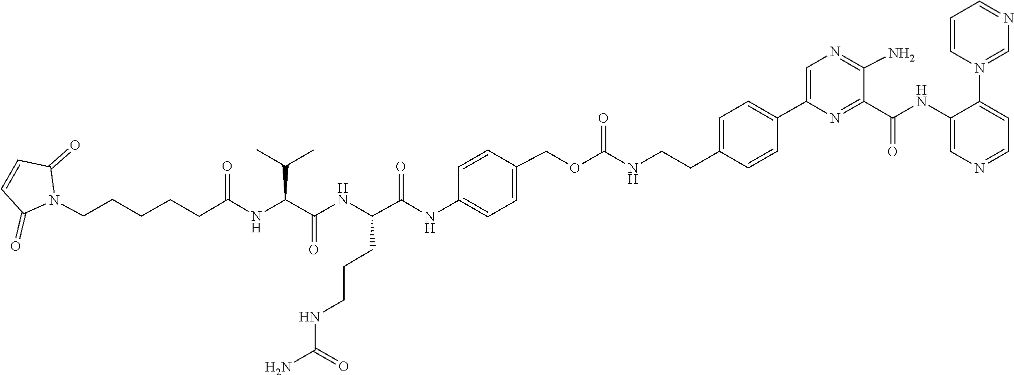 | 1010.6 |
| LP28 | 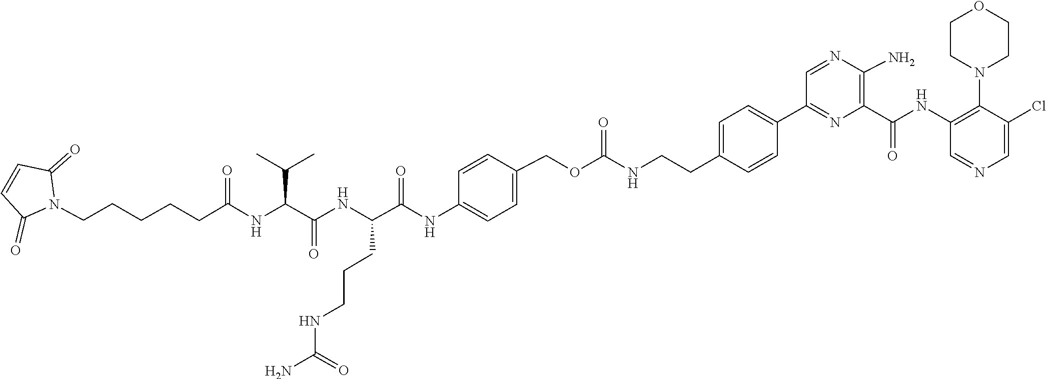 [SMILES: CC(C)[C@H](NC(=O)CCCCCN1C(=O)C=CC1=O)C(=O)N[C@@H](CCCNC(N)=O)C(=O)Nc1ccc(COC(=O)NCCc2ccc(-c3cnc(N)c(C(=O)Nc4cncc(Cl)c4N4CCOCC4)n3)cc2)cc1] | 1052.5 |
| LP29 | 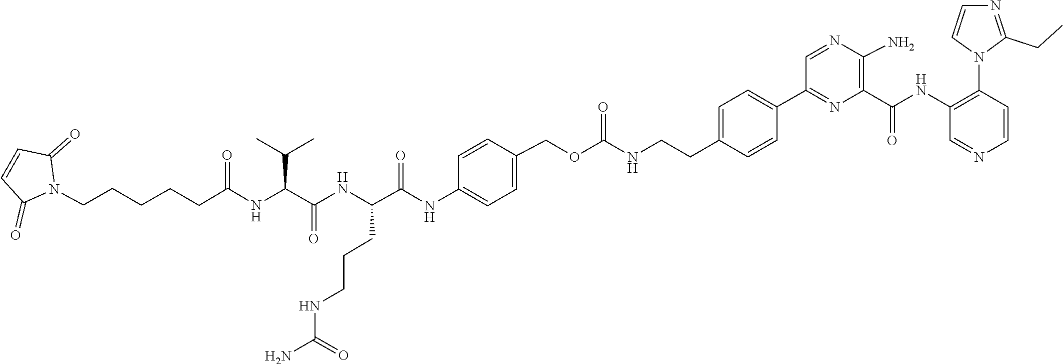 [SMILES: CCc1nccn1-c1ccncc1NC(=O)c1nc(-c2ccc(CCNC(=O)OCc3ccc(NC(=O)[C@H](CCCNC(N)=O)NC(=O)[C@@H](NC(=O)CCCCCN4C(=O)C=CC4=O)C(C)C)cc3)cc2)cnc1N] | 1027.6 |
| LP30 | 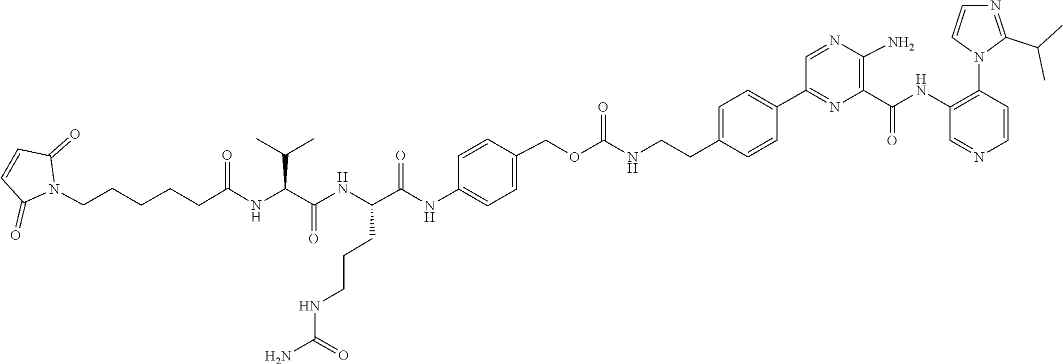 [SMILES: CC(C)c1nccn1-c1ccncc1NC(=O)c1nc(-c2ccc(CCNC(=O)OCc3ccc(NC(=O)[C@H](CCCNC(N)=O)NC(=O)[C@@H](NC(=O)CCCCCN4C(=O)C=CC4=O)C(C)C)cc3)cc2)cnc1N] | 1041.6 |
| LP31 | 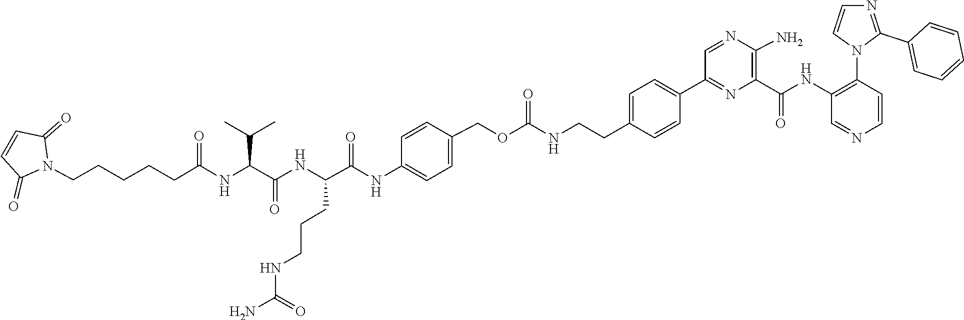 [SMILES: CC(C)[C@H](NC(=O)CCCCCN1C(=O)C=CC1=O)C(=O)N[C@@H](CCCNC(N)=O)C(=O)Nc1ccc(COC(=O)NCCc2ccc(-c3cnc(N)c(C(=O)Nc4cnccc4-n4ccnc4-c4ccccc4)n3)cc2)cc1] | 1075.6 |
| LP32 | 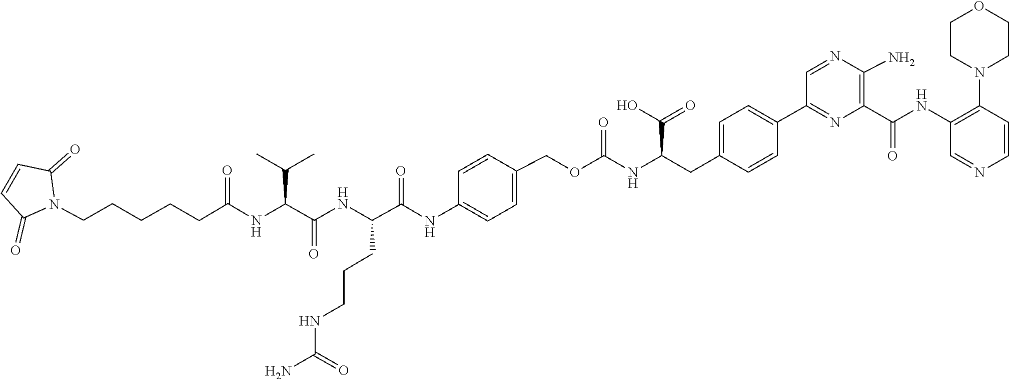 [SMILES: CC(C)[C@H](NC(=O)CCCCCN1C(=O)C=CC1=O)C(=O)N[C@@H](CCCNC(N)=O)C(=O)Nc1ccc(COC(=O)N[C@H](Cc2ccc(-c3cnc(N)c(C(=O)Nc4cnccc4N4CCOCC4)n3)cc2)C(=O)O)cc1] | 1062.6 |
| LP33 | 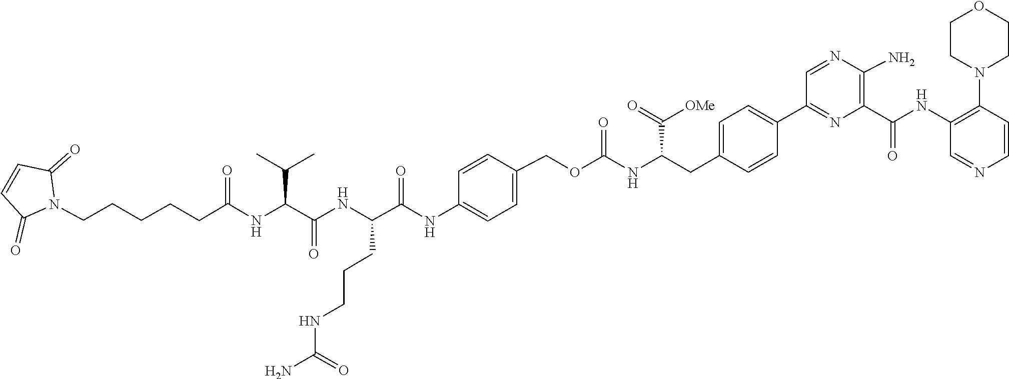 [SMILES: COC(=O)[C@H](Cc1ccc(-c2cnc(N)c(C(=O)Nc3cnccc3N3CCOCC3)n2)cc1)NC(=O)OCc1ccc(NC(=O)[C@H](CCCNC(N)=O)NC(=O)[C@@H](NC(=O)CCCCCN2C(=O)C=CC2=O)C(C)C)cc1] | 1076.6 |
| LP34 | 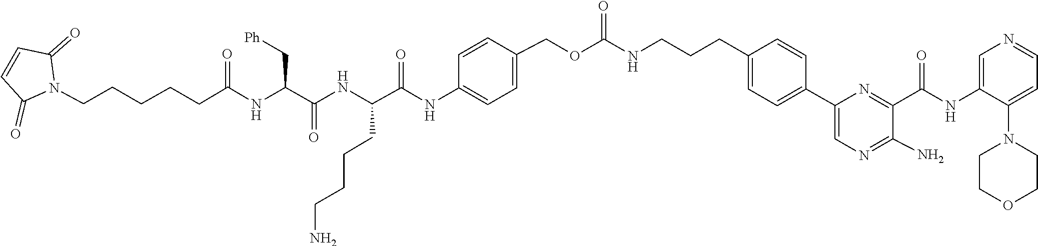 [SMILES: NCCCC[C@H](NC(=O)[C@H](Cc1ccccc1)NC(=O)CCCCCN1C(=O)C=CC1=O)C(=O)Nc1ccc(COC(=O)NCCCc2ccc(-c3cnc(N)c(C(=O)Nc4cnccc4N4CCOCC4)n3)cc2)cc1] | 1051.6 |
| LP35 | 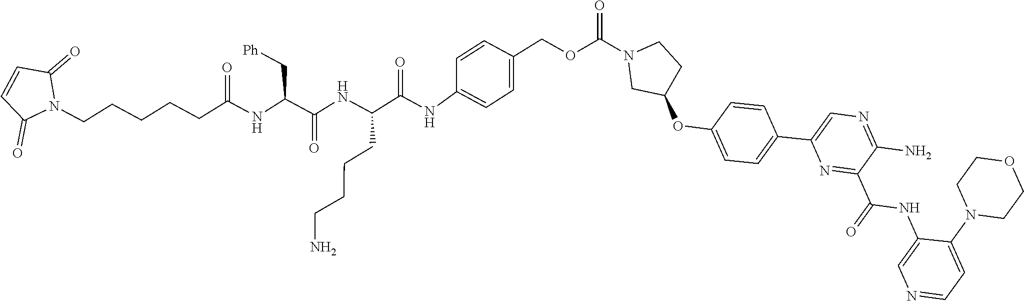 [SMILES: NCCCC[C@H](NC(=O)[C@H](Cc1ccccc1)NC(=O)CCCCCN1C(=O)C=CC1=O)C(=O)Nc1ccc(COC(=O)N2CC[C@@H](Oc3ccc(-c4cnc(N)c(C(=O)Nc5cnccc5N5CCOCC5)n4)cc3)C2)cc1] | 1079.6 |
| LP36 | 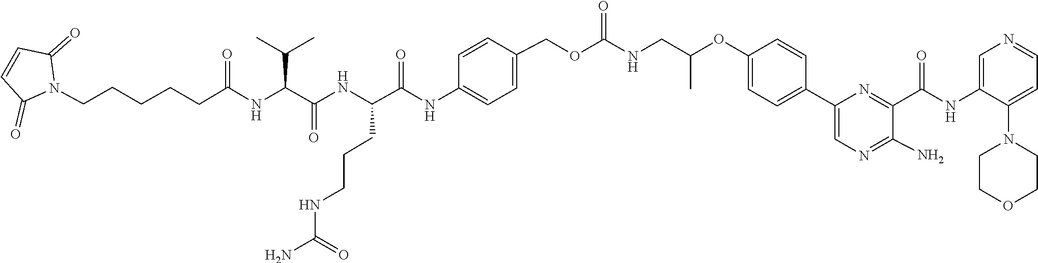 [SMILES: CC(CNC(=O)OCc1ccc(NC(=O)[C@H](CCCNC(N)=O)NC(=O)[C@@H](NC(=O)CCCCCN2C(=O)C=CC2=O)C(C)C)cc1)Oc1ccc(-c2cnc(N)c(C(=O)Nc3cnccc3N3CCOCC3)n2)cc1] | 1048.6 |

The linker-compounds in Table 17 were prepared using the teachings described herein in combination with the skill in the art

TABLE 17

| Com- | | |
|---|---|---|
| pound- | | |
| Linker | Structure | MS |
| LP2 | 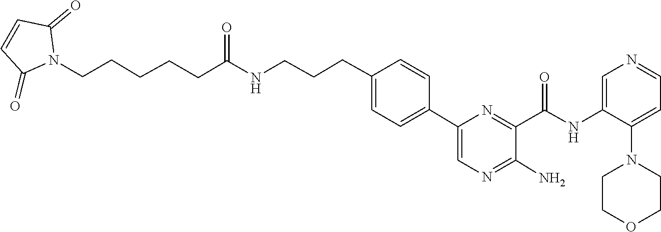 [SMILES: Nc1ncc(-c2ccc(CCCNC(=O)CCCCCN3C(=O)C=CC3=O)cc2)nc1C(=O)Nc1cnccc1N1CCOCC1] | [M + H]⁺: 627.4 |
| LP3 | 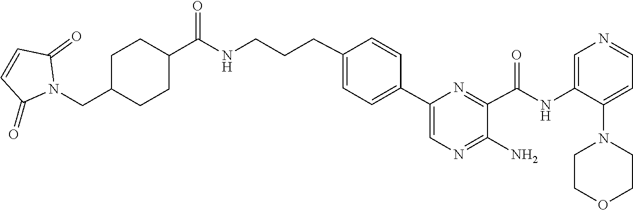 [SMILES: Nc1ncc(-c2ccc(CCCNC(=O)C3CCC(CN4C(=O)C=CC4=O)CC3)cc2)nc1C(=O)Nc1cnccc1N1CCOCC1] | [M + H]⁺: 653.4 |
| LP4 | 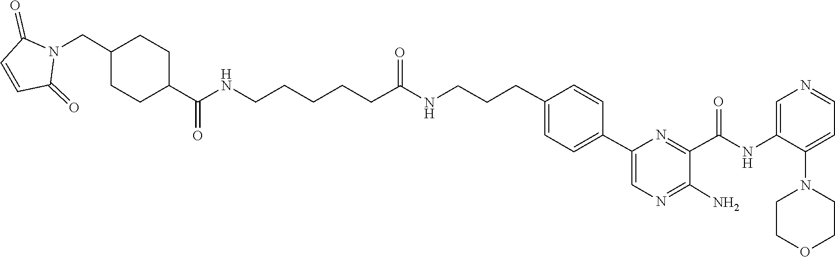 [SMILES: Nc1ncc(-c2ccc(CCCNC(=O)CCCCCNC(=O)C3CCC(CN4C(=O)C=CC4=O)CC3)cc2)nc1C(=O)Nc1cnccc1N1CCOCC1] | [M + H]⁺: 766.5 |
| LP5 | 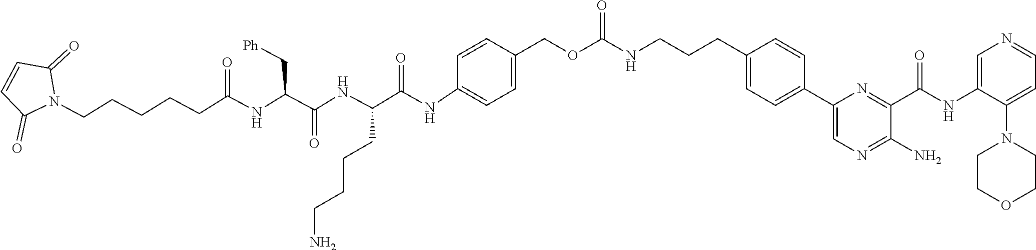 [SMILES: NCCCC[C@H](NC(=O)[C@H](Cc1ccccc1)NC(=O)CCCCCN1C(=O)C=CC1=O)C(=O)Nc1ccc(COC(=O)NCCCc2ccc(-c3cnc(N)c(C(=O)Nc4cnccc4N4CCOCC4)n3)cc2)cc1] | [M + H]⁺: 1051.6 |
| LP6 | 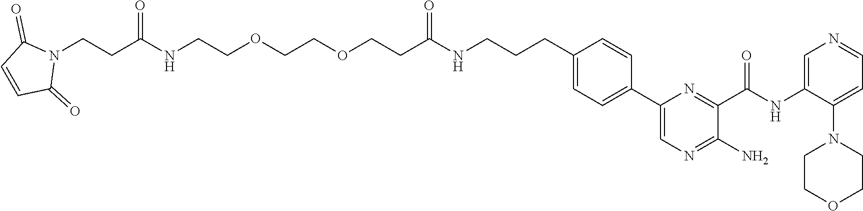 [SMILES: Nc1ncc(-c2ccc(CCCNC(=O)CCOCCOCCNC(=O)CCN3C(=O)C=CC3=O)cc2)nc1C(=O)Nc1cnccc1N1CCOCC1] | [M + H]⁺: 744.9 |
| LP7 | 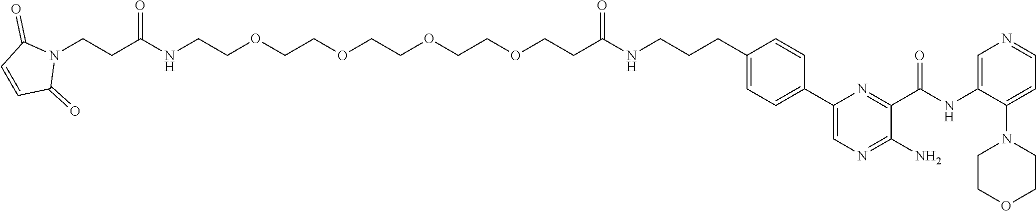 [SMILES: Nc1ncc(-c2ccc(CCCNC(=O)CCOCCOCCOCCOCCNC(=O)CCN3C(=O)C=CC3=O)cc2)nc1C(=O)Nc1cnccc1N1CCOCC1] | [M + H]⁺: 832.4 |
| LP8 | 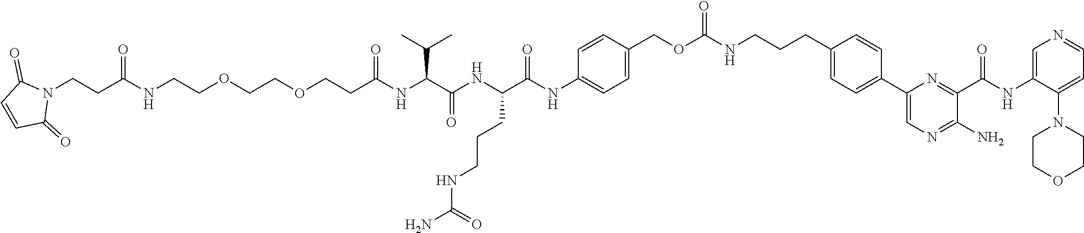 [SMILES: CC(C)[C@H](NC(=O)CCOCCOCCNC(=O)CCN1C(=O)C=CC1=O)C(=O)N[C@@H](CCCNC(N)=O)C(=O)Nc1ccc(COC(=O)NCCCc2ccc(-c3cnc(N)c(C(=O)Nc4cnccc4N4CCOCC4)n3)cc2)cc1] | [M + H]⁺: 1149.6 |
| LP37 | 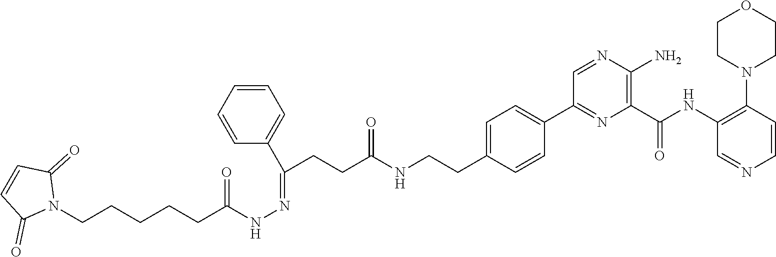 [SMILES: Nc1ncc(-c2ccc(CCNC(=O)CC/C(=N/NC(=O)CCCCCN3C(=O)C=CC3=O)c3ccccc3)cc2)nc1C(=O)Nc1cnccc1N1CCOCC1] ¹H NMR (300 MHz, Methanol-d₄) δ: 1.20-1.38 (m, 2H), 1.58-1.76 (m, 4H), 2.40- 2.53 (m, 4H), 2.70-2.87 (m, 2H), 3.01-3.10 (m, 6H), 3.45-3.52 (m, 4H), 3.79 (s, 4H), 6.75 (s, 2H), 7.27-7.38 (m, 6H), 7.84-7.96 (m, 2H), 7.99 (d, J = 1.5 Hz, 2H), 8.30 (d, J = 5.4 Hz, 1H), 8.79 (s, 1H), 9.49 (s, 1H). | |
| LP38 | 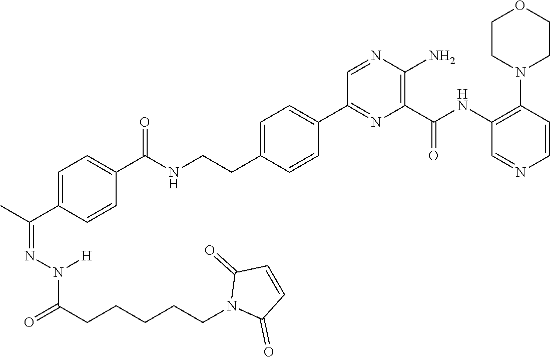 [SMILES: C/C(=N/NC(=O)CCCCCN1C(=O)C=CC1=O)c1ccc(C(=O)NCCc2ccc(-c3cnc(N)c(C(=O)Nc4cnccc4N4CCOCC4)n3)cc2)cc1] ¹H NMR (300 MHz, DMSO-d₆) δ: 1.21-1.39 (m, 3H), 1.41-1.71 (m, 4H), 2.25-2.27 (m, 3H), 2.51-2.71 (m, 2H), 2.97 (s, 6H), 3.55 (s, 3H), 3.73 (s, 4H), 6.98 (s, 1H), 7.01 (s, 1H), 7.22 (s, 1H), 7.41 (d, J = 8.1 Hz, 2H), 7.78 (s, 2H), 7.85 (d, J = 4.0 Hz, 4H), 8.15 (d, J = 8.1 Hz, 2H), 8.29 (s, 1H), 8.63 (s, 1H), 8.97 (s, 1H), 9.33 (s, 1H), 10.37 (s, 1H), 10.45 (s, 1H). | |
| LP39 | 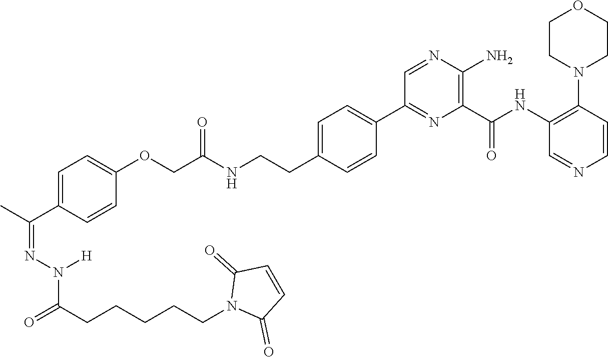 [SMILES: C/C(=N/NC(=O)CCCCCN1C(=O)C=CC1=O)c1ccc(OCC(=O)NCCc2ccc(-c3cnc(N)c(C(=O)Nc4cnccc4N4CCOCC4)n3)cc2)cc1] ¹H NMR (300 MHz, Methanol-d₄) δ: 1.33-1.35 (m, 2H), 1.59-1.62 (m, 4H), 2.12- 2.20 (m, 3H), 2.37-2.40 (m, 1H), 2.60-2.70 (m, 1H), 2.93-2.97 (m, 5H), 3.31-3.33 (m, 1H), 3.44-3.60 (m, 4H), 3.76-3.77 (m, 4H), 4.53 (s, 2H), 6.73 (s, 1H), 6.78 (s, 1H), 6.87-6.92 (m, 2H), 7.24 (d, J = 5.4 Hz, 1H), 7.32-7.36 (m, 2H), 7.66 (d, J = 8.9 Hz, 1H), 7.78 (d, J = 8.8 Hz, 1H), 7.91-7.96 (m, 2H), 8.26 (d, J = 5.4 Hz, 1H), 8.73 (d, J = 6.3 Hz, 1H), 9.52 (s, 1H). | |
| LP40 | 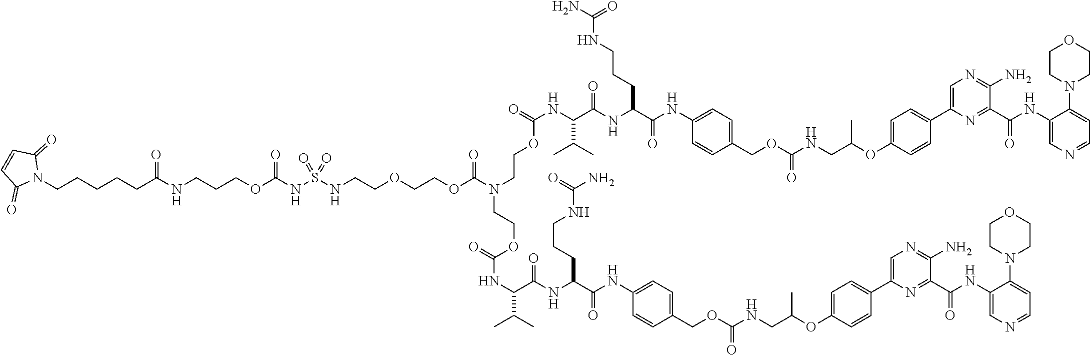 [SMILES: CC(CNC(=O)OCc1ccc(NC(=O)[C@H](CCCNC(N)=O)NC(=O)[C@@H](NC(=O)OCCN(CCOC(=O)N[C@H](C(=O)N[C@@H](CCCNC(N)=O)C(=O)Nc2ccc(COC(=O)NCC(C)Oc3ccc(-c4cnc(N)c(C(=O)Nc5cnccc5N5CCOCC5)n4)cc3)cc2)C(C)C)C(=O)OCCOCCNS(=O)(=O)NC(=O)OCCCNC(=O)CCCCCN2C(=O)C=CC2=O)C(C)C)cc1)Oc1ccc(-c2cnc(N)c(C(=O)Nc3cnccc3N3CCOCC3)n2)cc1] | [M + H]⁺: 2371.1 M/2: 1186.0 M/3: 791.0 |
| LP41 | 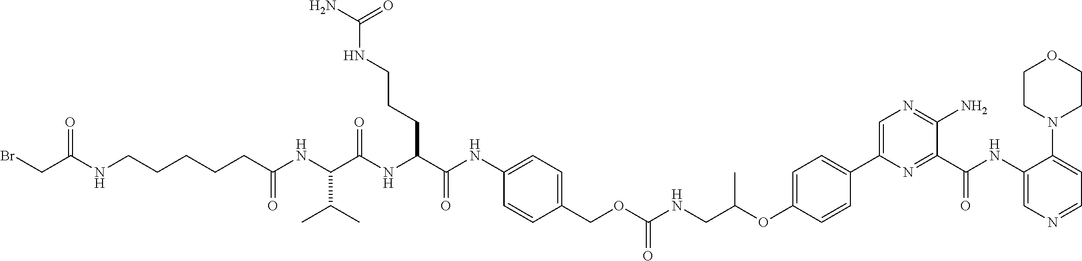 [SMILES: CC(CNC(=O)OCc1ccc(NC(=O)[C@H](CCCNC(N)=O)NC(=O)[C@@H](NC(=O)CCCCCNC(=O)CBr)C(C)C)cc1)Oc1ccc(-c2cnc(N)c(C(=O)Nc3cnccc3N3CCOCC3)n2)cc1] | [M + H]⁺: 1088.5 |
| LP42 | 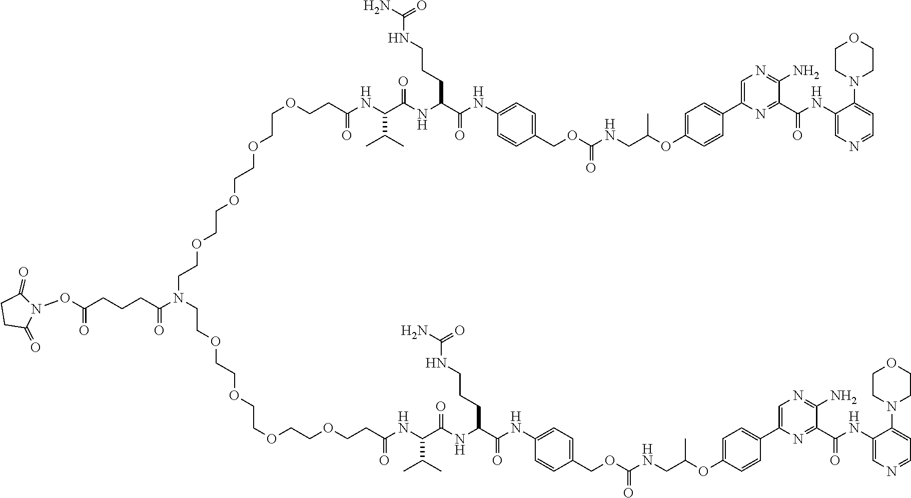 [SMILES: CC(CNC(=O)OCc1ccc(NC(=O)[C@H](CCCNC(N)=O)NC(=O)[C@@H](NC(=O)CCOCCOCCOCCOCCN(CCOCCOCCOCCOCCC(=O)N[C@H](C(=O)N[C@@H](CCCNC(N)=O)C(=O)Nc2ccc(COC(=O)NCC(C)Oc3ccc(-c4cnc(N)c(C(=O)Nc5cnccc5N5CCOCC5)n4)cc3)cc2)C(C)C)C(=O)CCCC(=O)ON2C(=O)CCC2=O)C(C)C)cc1)Oc1ccc(-c2cnc(N)c(C(=O)Nc3cnccc3N3CCOCC3)n2)cc1] | [M + H]⁺: 2398.2 M/2: 1199.6 M/3: 800.0 |
| LP43 | 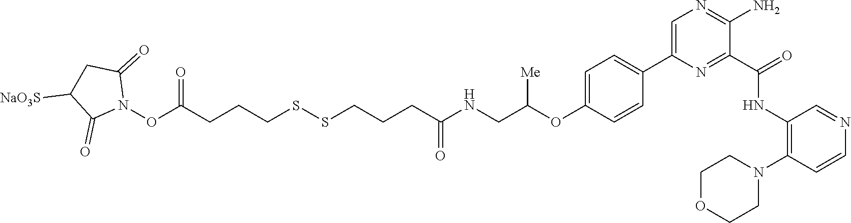 | [M + Na]⁺: 868.2 |
| LP44 | 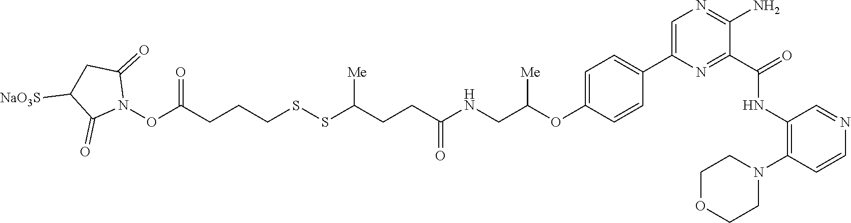 | [M + Na]⁺: 882.2 |
| LP45 | 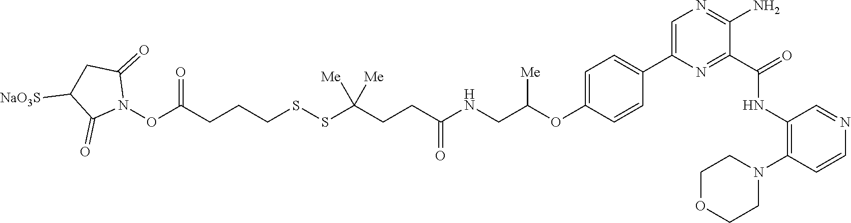 | [M + Na]⁺: 886.2 |
| LP46 | 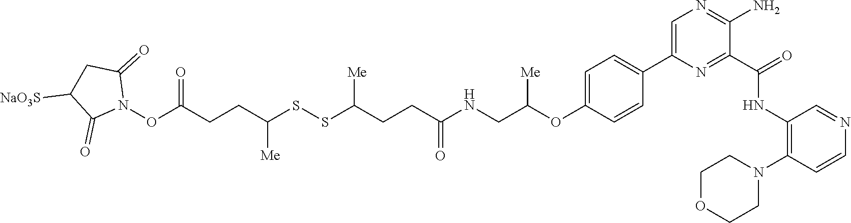 | [M + Na]⁺: 896.2 |
| LP47 | 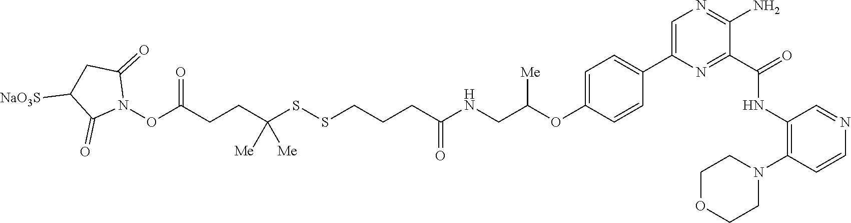 | [M + Na]⁺: 896.2 |
| LP48 | 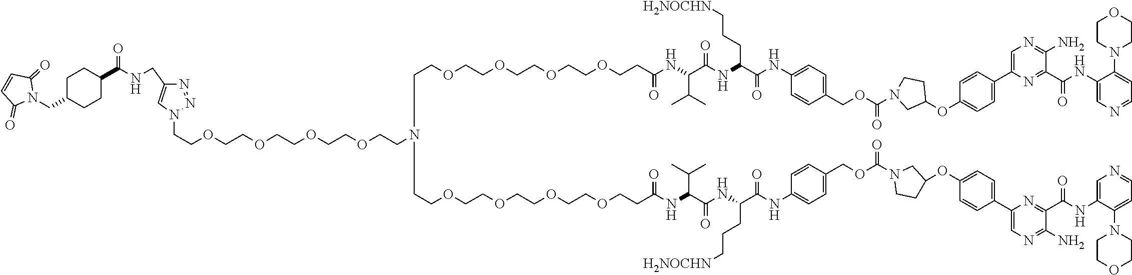 | [M + H]⁺: 2730.4 M/2: 1365.6 M/3: 910.8 |
| LP49 | 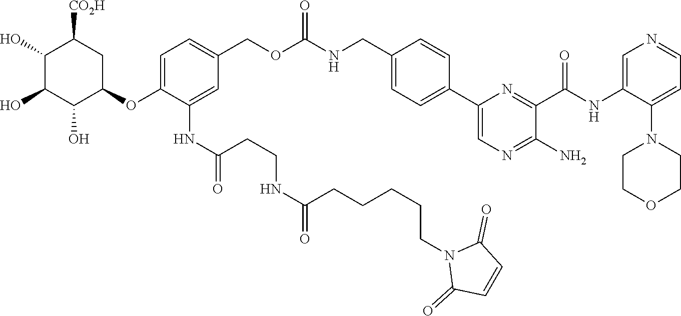 [SMILES: Nc1ncc(-c2ccc(CNC(=O)OCc3ccc(O[C@@H]4C[C@H](C(=O)O)[C@@H](O)[C@H](O)[C@H]4O)c(NC(=O)CCNC(=O)CCCCCN4C(=O)C=CC4=O)c3)cc2)nc1C(=O)Nc1cnccc1N1CCOCC1] | [M + H]⁺: 1011.4 |
| LP50 | 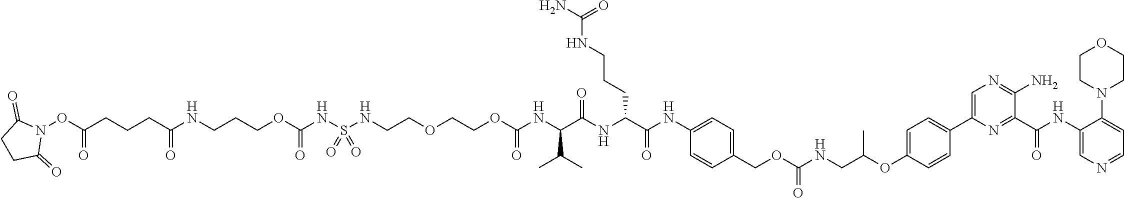 [SMILES: CC(CNC(=O)OCc1ccc(NC(=O)[C@@H](CCCNC(N)=O)NC(=O)[C@H](NC(=O)OCCOCCNS(=O)(=O)NC(=O)OCCCNC(=O)CCCC(=O)ON2C(=O)CCC2=O)C(C)C)cc1)Oc1ccc(-c2cnc(N)c(C(=O)Nc3cnccc3N3CCOCC3)n2)cc1] | [M + H]⁺: 1377.6 |
| LP51 | 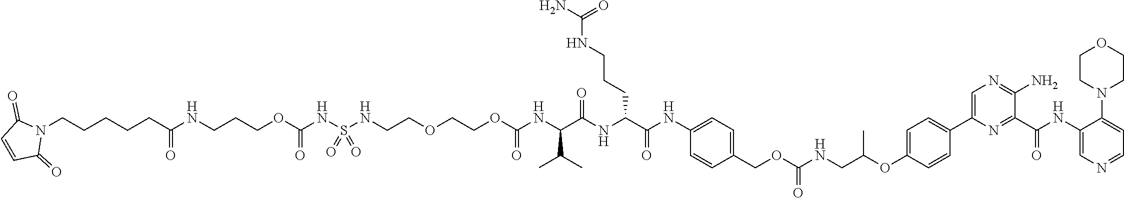 [SMILES: CC(CNC(=O)OCc1ccc(NC(=O)[C@@H](CCCNC(N)=O)NC(=O)[C@H](NC(=O)OCCOCCNS(=O)(=O)NC(=O)OCCCNC(=O)CCCCCN2C(=O)C=CC2=O)C(C)C)cc1)Oc1ccc(-c2cnc(N)c(C(=O)Nc3cnccc3N3CCOCC3)n2)cc1] | [M + H]⁺: 1359.6 |
| LP52 | 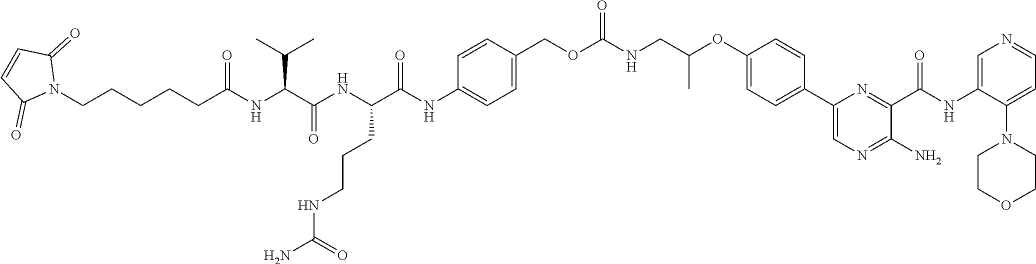 [SMILES: CC(CNC(=O)OCc1ccc(NC(=O)[C@H](CCCNC(N)=O)NC(=O)[C@@H](NC(=O)CCCCCN2C(=O)C=CC2=O)C(C)C)cc1)Oc1ccc(-c2cnc(N)c(C(=O)Nc3cnccc3N3CCOCC3)n2)cc1] | [M + H]⁺: 1048.6 |
| LP53 | 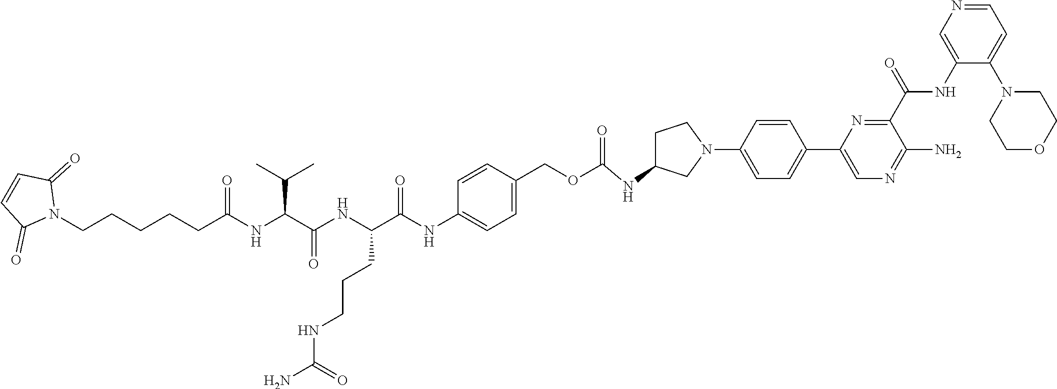 [SMILES: CC(C)[C@H](NC(=O)CCCCCN1C(=O)C=CC1=O)C(=O)N[C@@H](CCCNC(N)=O)C(=O)Nc1ccc(COC(=O)N[C@H]2CCN(c3ccc(-c4cnc(N)c(C(=O)Nc5cnccc5N5CCOCC5)n4)cc3)C2)cc1] | [M + H]⁺: 1059.6 |
| LP54 | 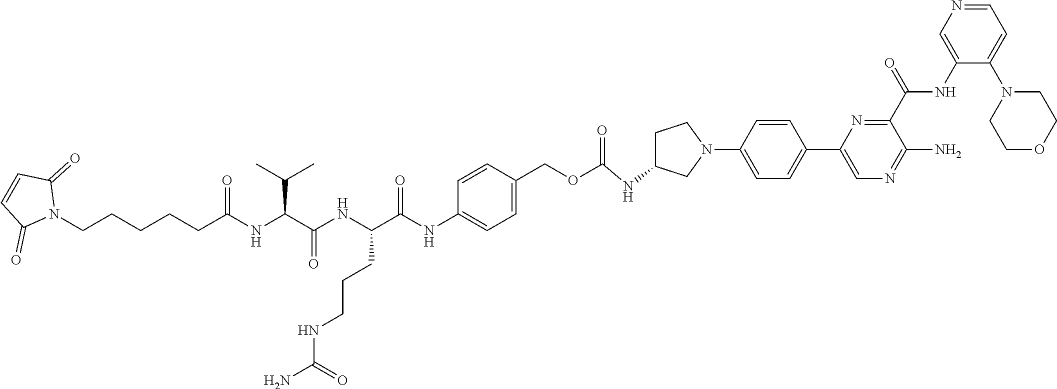 [SMILES: CC(C)[C@H](NC(=O)CCCCCN1C(=O)C=CC1=O)C(=O)N[C@@H](CCCNC(N)=O)C(=O)Nc1ccc(COC(=O)N[C@@H]2CCN(c3ccc(-c4cnc(N)c(C(=O)Nc5cnccc5N5CCOCC5)n4)cc3)C2)cc1] | [M + H]⁺: 1059.6 |
| LP55 | 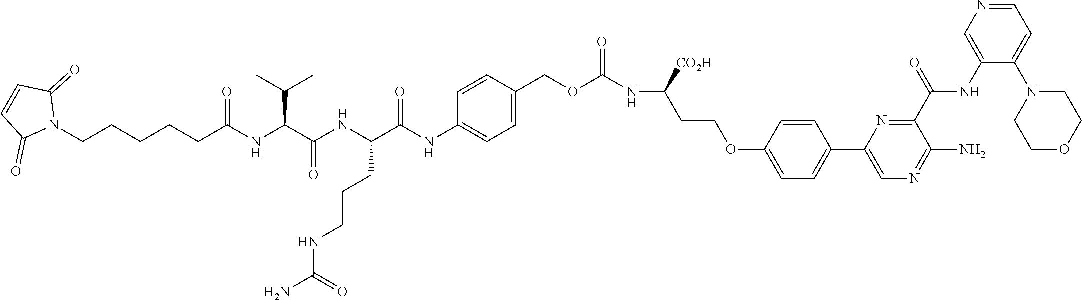 [SMILES: CC(C)[C@H](NC(=O)CCCCCN1C(=O)C=CC1=O)C(=O)N[C@@H](CCCNC(N)=O)C(=O)Nc1ccc(COC(=O)N[C@H](CCOc2ccc(-c3cnc(N)c(C(=O)Nc4cnccc4N4CCOCC4)n3)cc2)C(=O)O)cc1] | [M + H]⁺: 1092.6 |
| LP56 | 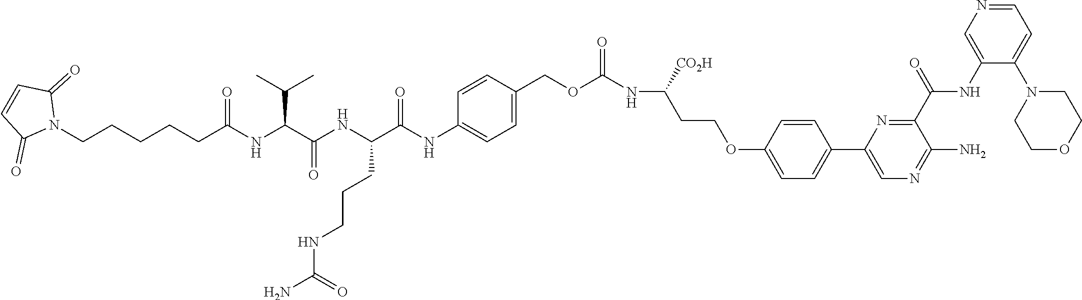 [SMILES: CC(C)[C@H](NC(=O)CCCCCN1C(=O)C=CC1=O)C(=O)N[C@@H](CCCNC(N)=O)C(=O)Nc1ccc(COC(=O)N[C@@H](CCOc2ccc(-c3cnc(N)c(C(=O)Nc4cnccc4N4CCOCC4)n3)cc2)C(=O)O)cc1] | [M + H]⁺: 1092.6 |
| LP57 | 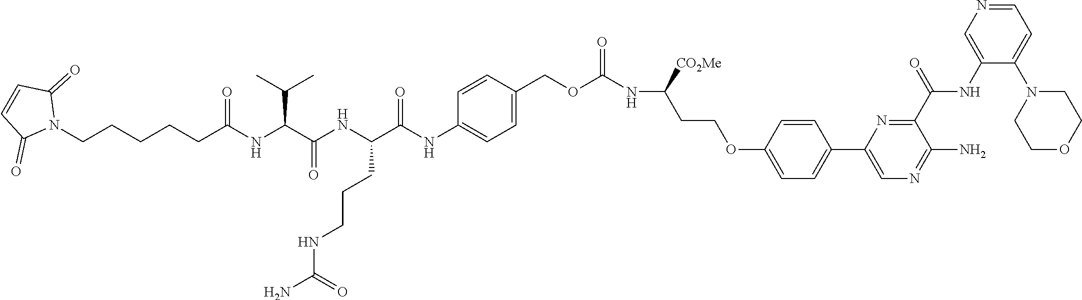 [SMILES: COC(=O)[C@@H](CCOc1ccc(-c2cnc(N)c(C(=O)Nc3cnccc3N3CCOCC3)n2)cc1)NC(=O)OCc1ccc(NC(=O)[C@H](CCCNC(N)=O)NC(=O)[C@@H](NC(=O)CCCCCN2C(=O)C=CC2=O)C(C)C)cc1] | [M + H]⁺: 1106.6 |
| LP58 | 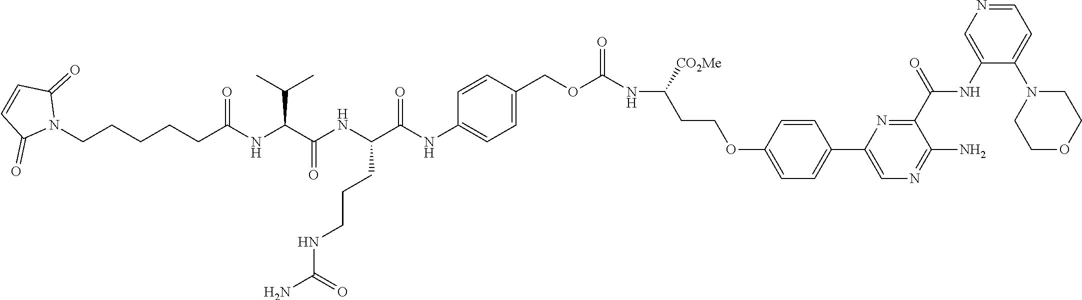 [SMILES: COC(=O)[C@H](CCOc1ccc(-c2cnc(N)c(C(=O)Nc3cnccc3N3CCOCC3)n2)cc1)NC(=O)OCc1ccc(NC(=O)[C@H](CCCNC(N)=O)NC(=O)[C@@H](NC(=O)CCCCCN2C(=O)C=CC2=O)C(C)C)cc1] | [M + H]⁺: 1106.6 |

### Example 7: Generation of Antibody-TGFb Inhibitor Conjugates Through Partial Reduction of Native Disulfide Bonds of Non-Engineered Antibodies

The mAb (3-8 mg/mL in PBS) was exchanged into HEPES (100 mM, pH 7.0, 1 mM DTPA) via molecular weight cut-off centrifugal filtration (Millipore, 30 kDa). The resultant mAb solution was transferred to a tared 50 mL conical tube. The mAb concentration was determined to be 3-8 mg/mL by A₂₈₀. To the mAb solution was added TCEP (2.0-4.0 equivalents, 1 mM stock) at room temperature and the resultant mixture was incubated at 37° C. for 30-90 minutes, with gentle shaking. Upon being cooled to room temperature, a stir bar was added to the reaction tube. With stirring, a linker-payload (5-10 equivalents, 10 mM DMSO) was added dropwise. The resultant reaction mixture was allowed to stir at ambient temperature for 30-60 minutes, at which point N-ethyl maleimide (3.0 equivalents, 100 mM DMA) was added. After an additional 15 minutes of stirring, N-acetylcysteine (6.0-11.0 equivalents, 50 mM HEPES) was added. The crude ADC was then exchanged into PBS and purified by preparative SEC (e.g. HiLoad 26/600, Superdex 200 pg) using PBS as the mobile phase. The pure fractions were concentrated via molecular weight cut-off centrifugal filtration (Millipore, 30 kDa), sterile filtered, and transferred to 15 mL conical tubes. Drug-antibody construct ratios (molar ratios) were determined by methods described in Example 8 below.

### Example 8: General Procedure for the Determination of the Drug-Antibody-Ratios Hydrophobic Interaction Chromatography

10 μL of a 6 mg/mL solution of a conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six-minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 9: TGFβ Reporter Assay

### Materials and General Procedures

TGFβ/SMAD Signaling Pathway SBE reporter cell line was obtained from BPS Bioscience. Cells were passed, expanded, and stored in liquid nitrogen as per the supplier's instructions with the exception that growth media is changed to DMEM-C with Geneticin (DMEM supplemented with 10% fetal bovine serum, 1×NEAA, 1 mM Pyruvate, 2 mM glutamine, 50 μg/mL penicillin, 50 U/mL streptomycin and 400 μg/mL of Geneticin). The assay media was MEM supplemented with 0.5% fetal bovine serum, 1×NEAA, 1 mM Pyruvate, 50 μg/mL penicillin and 50 U/mL streptomycin.

### General Procedure for In Vitro Small Molecule Screening

Test samples (at desired concentrations diluted in assay media) were added to a 96-well assay plate, 20 μL per well. Reporter cells were harvested from the tissue culture flasks by incubation in small quantity of PBS at 37° C. for two minutes after the media in the flask is removed and cells rinsed with PBS. Cells were counted and diluted in the assay media at approximately 0.5×10⁶ cells/mL and then 80 μL/well of cells were added to the assay plate containing the 20 μL/well of test samples (or media only), and incubated for approximately 5-6 hours at 37° C. in a 5% CO₂ humidified incubator. After that time, 15 μL of TGFβ diluted to 12 ng/mL in the assay media was added to the plate. Controls included TGFβ titration (from 50 to 0 ng/mL) without inhibitors, and media only (without cells, inhibitor or TGFβ). Plates were incubated at 37° C. in a 5% CO₂ humidified incubator for 18 h. Luciferase substrate solution is subsequently added at 100 μL per well, incubated in the dark at room temperature for 15 min, and luminescence is measured using a luminometer. EC₅₀ values and curve fits were obtained using Prism (GraphPad Software).

Table 18 includes EC₅₀ values for selected compounds; with compounds having an EC₅₀ value <50 nM as AA, 50 nM to 100 nM as A, 100 nM to 1000 nM as B, and greater than 1000 nM as C.

Compounds of the disclosure were also assayed by Reaction Biology Corp. using the TGFBR2 RBC assay (enzyme inhibition assay). Table 18 also includes IC₅₀ values for selected compounds; with compounds having an IC₅₀ value <50 nM as AA, 50 nM to 100 nM as A, 100 nM to 1000 nM as B, and greater than 1000 nM as C. NT refers to not tested.

TABLE 18

| | EC₅₀ | IC₅₀ |
|---|---|---|
| Compound | (reporter assay) | (enzyme inhibition assay) |
| 1.1 | B | AA |
| 1.2 | B | AA |
| 2.1 | A | AA |
| 3.1 | B | AA |
| 4.1 | B | AA |
| 5.1 | B | AA |
| 6 | A | AA |
| 7 | A | AA |
| 8 | B | NA |
| 9 | B | NT |
| 10 | C | NT |
| 11 | C | NT |
| 12 | B | NT |
| 13 | C | NT |
| 14 | C | C |
| 15 | B | B |
| 16 | B | AA |
| 17 | B | B |
| 18 | B | C |
| 19 | C | C |
| 20 | C | C |
| 21 | B | C |
| 22 | A | B |
| 23 | B | AA |
| 24 | B | B |
| 25 | B | AA |
| 26 | B | AA |
| 27 | C | C |
| 28 | B | B |
| 29 | B | C |
| 30 | C | B |
| 31 | C | C |
| 32 | A | B |
| 33 | B | C |
| 34 | C | B |
| 35 | B | B |
| 36 | A | B |
| 37 | C | C |
| 38 | B | B |
| 39 | C | B |
| 40 | C | AA |
| 41 | B | B |
| 42 | B | A |
| 43 | B | AA |
| 44 | B | B |
| 45 | NT | AA |
| 46 | B | AA |
| 47 | C | C |
| 48 | C | AA |
| 49 | C | C |
| 50 | B | AA |
| 51 | A | AA |
| 52 | B | C |
| 53 | C | C |
| 54 | A | AA |
| 55 | AA | AA |
| 56 | A | A |
| 57 | B | B |
| 58 | B | NT |
| 59 | NT | NT |
| 60 | B | NT |
| 616 | B | NT |
| 62 | A | B |
| 63 | A | B |
| 64 | A | AA |
| 65 | B | AA |
| 66 | C | B |
| 67 | A | A |
| 68 | B | AA |
| 69 | C | C |
| 70 | A | AA |
| 71 | B | AA |
| 72 | B | A |
| 73 | A | AA |
| 74 | B | A |
| 75 | B | AA |
| 76 | B | AA |
| 77 | A | A |
| 78 | AA | AA |
| 79 | B | C |
| 80 | B | A |
| 81 | A | B |
| 83 | B | A |
| 83 | A | B |
| 84 | B | B |
| 85 | AA | AA |
| 86 | B | AA |
| 87 | A | AA |
| 88 | A | AA |
| 89 | A | AA |
| 90 | A | AA |
| 91 | A | A |
| 92 | B | AA |
| 93 | B | B |
| 94 | B | AA |
| 95 | C | C |
| 96 | C | C |
| 97 | C | C |
| 98 | C | C |
| 99 | C | NT |
| 100 | C | NT |
| 101 | B | A |
| 102 | B | B |
| 103 | B | AA |
| 104 | C | C |
| 105 | B | B |
| 106 | B | A |
| 107 | B | AA |
| 108 | A | A |
| 109 | B | AA |
| 110 | B | AA |
| 111 | B | AA |
| 112 | A | AA |
| 113 | C | C |
| 114 | C | C |
| 115 | C | C |
| 116 | B | B |
| 117 | C | B |
| 118 | C | C |
| 119 | B | AA |
| 120 | C | AA |
| 121 | B | AA |
| 122 | C | C |
| 123 | C | C |
| 124 | B | B |
| 125 | C | B |
| 126 | B | C |
| 127 | B | AA |
| 128 | B | AA |
| 129 | B | A |
| 130 | C | C |
| 131 | C | C |
| 132 | B | AA |
| 133 | C | AA |
| 134 | B | AA |
| 135 | C | C |
| 136 | C | AA |
| 137 | C | AA |
| 138 | C | AA |
| 139 | B | AA |
| 140 | C | AA |
| 141 | C | AA |
| 142 | B | AA |
| 143 | C | AA |
| 144 | C | AA |
| 145 | NT | B |
| 146 | NT | B |
| 147 | NT | A |
| 148 | C | B |
| 149 | C | A |
| 150 | C | B |
| 151 | B | A |
| 152 | B | AA |
| 153 | C | B |
| 154 | C | B |
| 155 | B | AA |
| 156 | B | AA |
| 157 | B | B |
| 158 | B | AA |
| 159 | B | B |
| 160 | B | AA |
| 161 | B | B |
| 162 | B | AA |
| 163 | B | B |
| 164 | B | A |
| 165 | B | B |
| 166 | B | B |
| 167 | A | AA |
| 168 | A | AA |
| 169 | A | AA |
| 170 | A | AA |
| 171 | A | AA |
| 172 | C | C |
| 173 | B | C |
| 174 | B | AA |
| 175 | B | B |
| 176 | B | AA |
| 177 | B | A |
| 178 | B | AA |
| 179 | C | A |
| 180 | B | A |
| 181 | B | AA |
| 182 | B | AA |
| 183 | B | AA |
| 184 | B | AA |
| 185 | B | AA |
| 186 | C | C |
| 187 | B | AA |
| 188 | C | AA |
| 189 | C | AA |
| 190 | B | A |
| 191 | C | AA |
| 192 | B | AA |
| 193 | A | AA |
| 194 | B | AA |
| 195 | B | AA |
| 196 | B | AA |
| 197 | C | B |
| 198 | B | A |
| 199 | C | AA |
| 200 | C | AA |
| 201 | B | B |
| 202 | B | AA |
| 203 | C | AA |
| 204 | C | AA |
| 205 | B | AA |
| 206 | B | AA |
| 207 | B | AA |
| 208 | C | AA |
| 209 | B | AA |
| 210 | B | AA |
| 211 | AA | AA |
| 212 | B | AA |
| 213 | A | AA |
| 214 | B | AA |
| 215 | B | AA |
| 216 | A | AA |
| 217 | B | AA |
| 218 | A | AA |
| 219 | A | AA |
| 220 | B | AA |
| 221 | AA | AA |
| 222 | B | AA |
| 223 | B | AA |
| 224 | C | AA |
| 225 | B | B |
| 226 | B | AA |
| 227 | A | AA |
| 228 | B | A |
| 229 | B | A |
| 230 | A | AA |
| 231 | C | NT |
| 232 | C | AA |
| 233 | B | AA |
| 234 | B | AA |
| 235 | B | AA |
| 236 | C | AA |
| 237 | A | AA |
| 238 | B | A |
| 239 | B | A |
| 240 | C | A |
| 241 | C | B |
| 242 | C | A |
| 243 | B | B |
| 244 | B | AA |
| 245 | B | AA |
| 246 | B | AA |
| 247 | A | AA |
| 248 | B | AA |
| 249 | B | AA |
| 250 | B | AA |
| 251 | B | AA |
| 252 | B | AA |
| 253 | C | B |
| 254 | C | C |
| 255 | C | C |
| 256 | B | A |
| 257 | C | AA |
| 258 | AA | AA |
| 259 | AA | AA |
| 260 | AA | AA |
| 261 | C | C |
| 262 | B | AA |
| 263 | B | AA |
| 264 | C | AA |
| 265 | C | AA |
| 266 | B | AA |
| 267 | B | AA |
| 268 | C | AA |
| 269 | B | AA |
| 270 | B | AA |
| 271 | B | AA |
| 272 | A | AA |
| 273 | B | AA |
| 274 | B | AA |
| 275 | C | A |
| 276 | C | AA |
| 277 | A | AA |
| 278 | A | AA |
| 279 | A | AA |
| 280 | B | AA |
| 281 | B | NT |
| 282 | B | NT |
| 283 | B | NT |
| 284 | B | NT |
| 285 | A | NT |
| 286 | B | NT |
| 287 | B | NT |
| 288 | B | NT |
| 289 | A | NT |
| 290 | AA | NT |
| 291 | B | NT |
| 292 | B | NT |
| 293 | B | NT |
| 294 | B | NT |
| 295 | B | NT |
| 296 | AA | NT |
| 297 | B | NT |
| 298 | B | NT |
| 399 | AA | NT |
| 300 | B | NT |
| 301 | A | NT |

### Example 10-21

In Examples 10-21, the LRRC15 antibody is the murine M25 antibody or a humanized variant thereof (see International Application No. WO2017/095805, incorporated herein by reference in its entirety and for all purposes). Conjugation to the linker-payload is via the interchain disulfides. The antibodies have either a wild-type Fe domain or a null Fe domain. The Fcnull mutations for human IgG1 are L234A, L236A, G237A, and K322A and the Fcnull mutations for murine IgG2a are L234A, L236A, G237A, K322A, and P329G; numbering by EU index. Linker-payloads are as shown in Tables 15-17.

### Example 10: Multiple Antibody-TGFb Inhibitor Conjugates Reduce TGFβ-Induced SMAD2 Activity

A set of TGFβR2 inhibitor molecules conjugated to an anti-LRRC15 antibody by a cathepsin B cleavable PABC linker at an average DAR (drug loading) of 2.5-4 were tested for their ability to decrease TGFβ 1-induced SMAD2 promoter activity in target cells expressing the antibody antigen. The LP1 and LP10 linker-payloads were conjugated to a humanized IgG1 M25 antibody with a Fc null domain while the other linker-payloads were conjugated to the murine IgG2a M25 antibody with a Fc null domain. HEK293 SMAD2p luciferase reporter cells (BPS Bioscience Inc.) transfected to stably express full length human LRRC15 were seeded in 96 well plates at 40,000 cells/well in an assay media of MEM+0.5% FBS, 1% NEAA, 1% NaPyr & 1% Pen/Strep. Conjugates and controls were added to wells in a dose titration ranging from 500 nM to 0.03 nM. After 24 hours of culture at 37 C in a 5% CO₂ environment human TGFβ1 was added (PeproTech Inc.) to a final concentration of 1.6 ng/ml followed by an additional 18 hours of culture. Luciferase Steady Glo reagent (Promega Corporation) was added as recommended by manufacturer. After incubating 10 minutes with shaking, SMAD2p activity was determined by measuring luminescence with an Envision Plate Reader (Perkin-Elmer Inc.) and an absolute IC50 was determined using Prism Software v8.1.0 (GraphPad Inc.). Many small molecule anti-LRRC15 antibody conjugates reduced the chemiluminescent signal thereby demonstrating inhibition of the TGFβ-induced signal. The dependence on antigen binding for inhibition was demonstrated both by lack of inhibition by an irrelevant non-antigen binding anti-Digoxin antibody conjugate as well as lack of inhibition on the parental HEK293 SMAD2-luciferase reporter line by several potent anti-LRRC15 conjugates (Data not shown). Potency classes are shown in Table 19 below.

TABLE 19
LRRC15 Conjugates

| <10 nM IC50 | >10 nM < 100 nM IC50 | >100 nM |
|---|---|---|
| mAb-LP1, | mAb-LP21, | mAb-LP27, |
| mAb-LP10, | mAb-LP22, | mAb-LP28, |
| mAb-LP11 | mAb-LP23, | mAb-LP29, |
| mAb-LP12, | mAb-LP24, | mAb-LP30, |
| mAb-LP13, | mAb-LP25, | mAb-LP31, |
| mAb-LP14, | mAb-LP26, | mAb-LP32, |
| mAb-LP15, | | mAb-LP33 |
| mAb-LP16, | | |
| mAb-LP17, | | |
| mAb-LP18, | | |
| mAb-LP19, | | |
| mAb-LP20 | | |

### Example 11: An Exemplary TGFβR2 Inhibitor Conjugated to a Monoclonal Antibody with Different Linkers can Reduce TGFβ-Induced SMAD2 Activity

Conjugates of compound 2.1 covalently attached to an anti-LRRC15 antibody with different cleavable or non-cleavable linkers were tested for their ability to lower SMAD2 promoter activity induced by TGFβ using the HEK293 SMAD2-luciferase reporter cell line expressing human LRRC15.

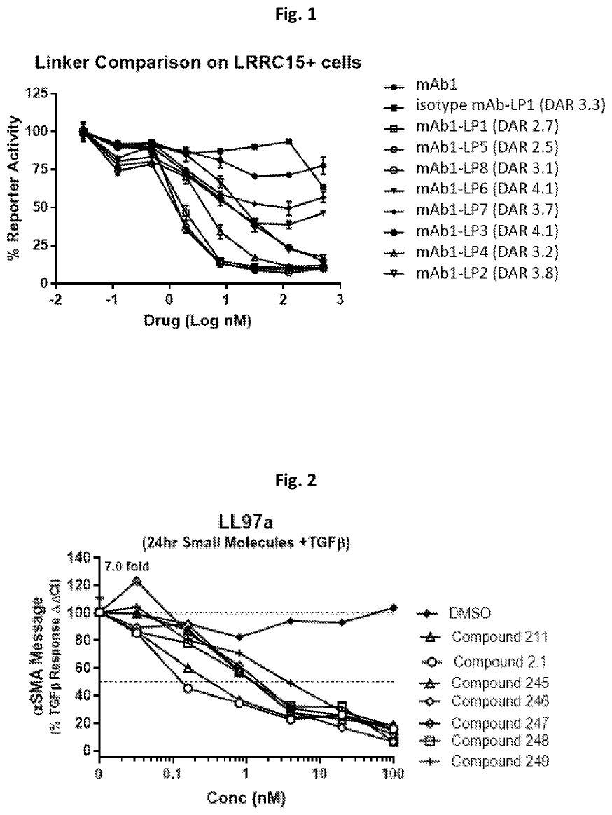

The linker-payloads were conjugated to a humanized IgG1 M25 antibody with a wild-type Fc domain. HEK293 SMAD2p luciferase reporter cells (BPS Bioscience Inc.) transfected to stably express full length human LRRC15 were seeded in 96 well plates at 40,000 cells/well in an assay media of MEM+0.5% FBS, 1% NEAA, 1% NaPyr & 1% Pen/Strep. Conjugates and controls were added to wells in a dose titration ranging from 500 nM to 0.03 nM. After 24 hours of culture at 37 C in a 5% CO₂ environment human TGFβ1 was added (PeproTech Inc.) to a final concentration of 1.6 ng/ml followed by an additional 18 hour of culture. Luciferase Steady Glo reagent (Promega Corporation) was added as recommended by manufacturer. After incubating 10 minutes with shaking, SMAD2p activity was determined by measuring luminescence with an Envision Plate Reader (Perkin-Elmer Inc.) and an absolute IC50 was determined using Prism Software v8.01 (GraphPad Inc.). The cleavable linkers all displayed potency with IC50<1 nM while the non-cleavable linkers displayed more variable potency. Dependency on LRRC15 binding was again demonstrated by minimal inhibition of the TGFβ-induced increase in SMAD2p activity by the anti-digoxin conjugate (isotype mAb-LP1). See FIG. 1.

### Example 12: Exemplary TGFβR2 Inhibitor Molecules Reduce a TGFβ-Induced mRNA in a Human IPF Patient Derived Fibroblast Cell Line in Concentration Dependent Manner

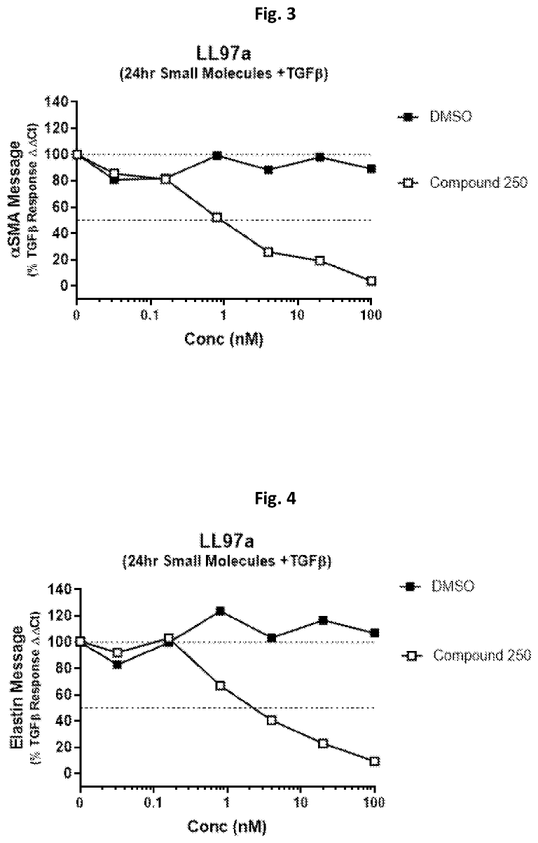

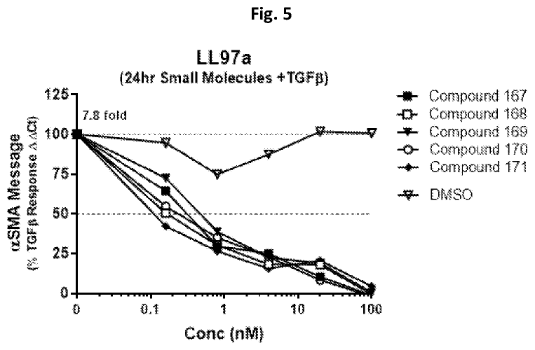

The ability of select small molecules to lower a TGFβ 1-induced mRNA were tested in assays with the cell line LL97a (ATCC) as follows. LL97a cells were seeded in 96 well plates at 10,000 cells/well in DMEM+10% FBS+1% NaPyr, +1% L-glut, +1% NEAA, +1% HEPES+0.5% Pen/Strep. The following day test articles and controls were added at varying concentrations in assay media of DMEM+0.5% BSA along with 0.5 ng/mL TGFβ1 (PeproTech Inc.) was added to wells. After 24 hours or 48 hours incubation with TGFβ1 at 37 C in a 5% CO₂ environment RNA was prepared from cells and QPCR performed for the TGFβ-inducible genes αSMA and elastin as well as the non-inducible control gene RPLPO with a Tagman® Gene Expression Cells-to-C_{T} 1—Step Kit (Life Technologies Inc.) and with a One Step Plus Thermocycler (Applied Biosystems) as per manufacturers' recommendations. Primer probe sets were obtained from ThermoFisher Scientific. As shown in FIGS. 2-5, TGFβR2 inhibitors potently inhibited the TGFβ-induced genes in a concentration dependent manner.

### Example 13: TGFβR2 Inhibitors Conjugated to an Anti-LRRC15 Antibody Show Potent Inhibition of TGFβ-Induced Gene Expression

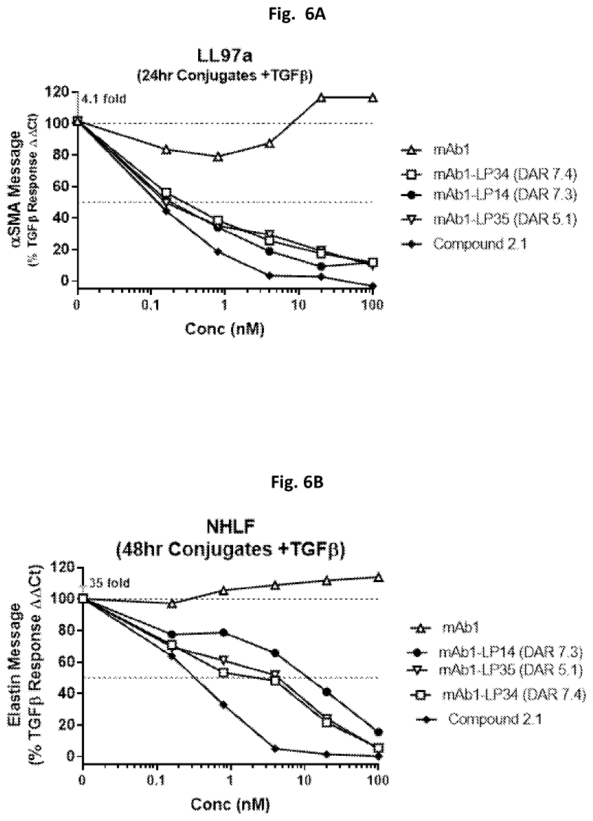

Compounds 2.1 and 171 were covalently attached to an anti-LRRC15 antibody using different protease cleavable PABC linkers. The linker-payloads were conjugated to a murine IgG2a M25 antibody with Fc null domain. The conjugates were purified bypreparative size exclusion chromatography. Average DAR level of the purified species was determined by analysis via hydrophobic interaction chromatography (HIC) using TSK-gel butyl NPR (Tosoh Bioscience LLC) and by LCMS (Waters Corp.) and they were tested for their ability to reduce αSMA and elastin gene induction by TGFβ in the LL97a and NHLF human fibroblast cell lines using a qPCRassay. For the assay, 10,000 cells of LL97a or NHLF were placed in wells of 6-well plates in their recommended growth media and incubated ON. The growth media was removed and replaced with assay media, DMEM+0.5% BSA (LL97a) or FBM+0.1% BSA (NHLF), to which varying concentrations of test articles and controls were added to the wells along with TGFβ1 (PeproTech Inc.) to a final concentration of 0.5 ng/ml. After incubation at 37 C in 5% CO₂ environment for 24 hours or 48 hours, RNA was prepared from the wells and Taqman® qPCR performed using a Cells-to-C_{T} 1—Step Kit (Life Technologies Inc.) per manufacturer's recommendation using probe sets for αSMA and elastin with a Step One Plus thermocycler (Applied Biosystems). As shown in FIGS. 6A and 6B, different linkers supported effective antibody delivery of the inhibitors into cells for both LL97a cells (A) and normal fibroblasts (B) as evidenced by potent reduction of mRNAs for αSMA and elastin.

### Example 14: Conjugates of a TGFbR2 Inhibitor to an Anti-LRRC15 Antibody with an Fc Domain with No FcγR Binding or an Fc Domain with Full FcγR Binding have Equipotency

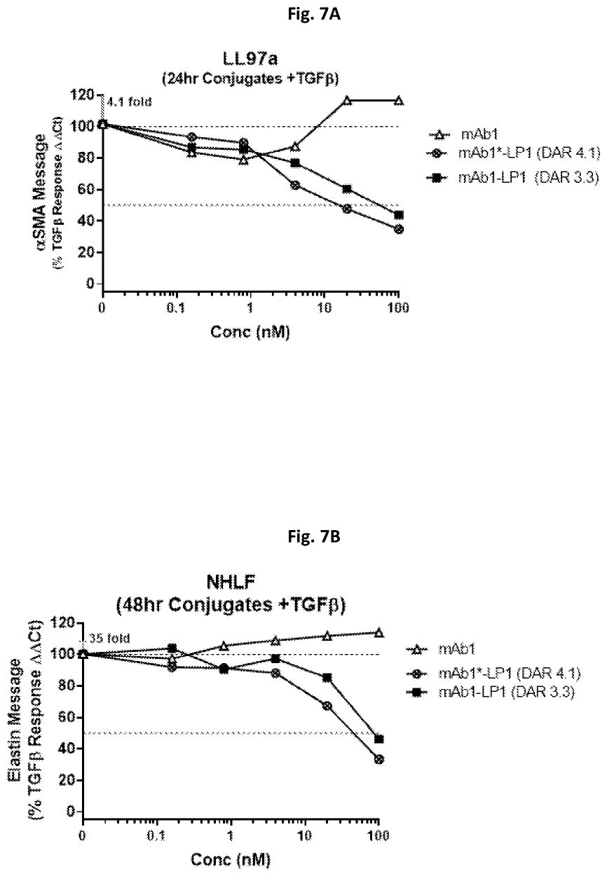

Compound 2.1 was covalently attached via a PABC linker to a murine IgG2a M25 antibody that carried either a wild-type Fc domain or an Fc null domain. Fcnull mutations for murine IgG2a are L234A, L236A, G237A, K322A, and P329G based on EU numbering, thereby lacking detectible FcγR binding by a biolayer interferometry assay (Octet; Forte Bio) (Fcnull). For the assay, 10,000 cells of LL97a or NHLF were placed in wells of 6-well plates in their recommended growth media and incubated ON. The growth media was removed and replaced with assay media, DMEM+0.5% BSA (LL97a) or FBM+0.1% BSA (NHLF), to which varying concentrations of test articles and controls were added to the wells along with TGFβ1 (PeproTech Inc.) to a final concentration of 0.5 ng/ml. After incubation at 37 C in 5% CO₂ environment for 24 hours or 48 hours, RNA was prepared from the wells and Taqman® qPCR performed using a Cells-to-C_{T} 1—Step Kit (Life Technologies Inc.) per manufacturer's recommendation using probe sets for αSMA and elastin with a Step One Plus thermocycler (Applied Biosystems). As shown in FIG. 7, at an approximately equal average DAR the conjugates had comparably equal ability to reduce the for αSMA and elastin in both LL97a cells FIG. 7 (A) and the normal fibroblasts (B). The asterisk denotes the antibody that has a wild-type Fc domain

### Example 15: Intra-Tumoral Injection of TGFβR2 Inhibitors Reduce Gene Expression of Disease Relevant TGFβ-Inducible Genes in Two Xenograft Models of Human Pancreatic Cancer

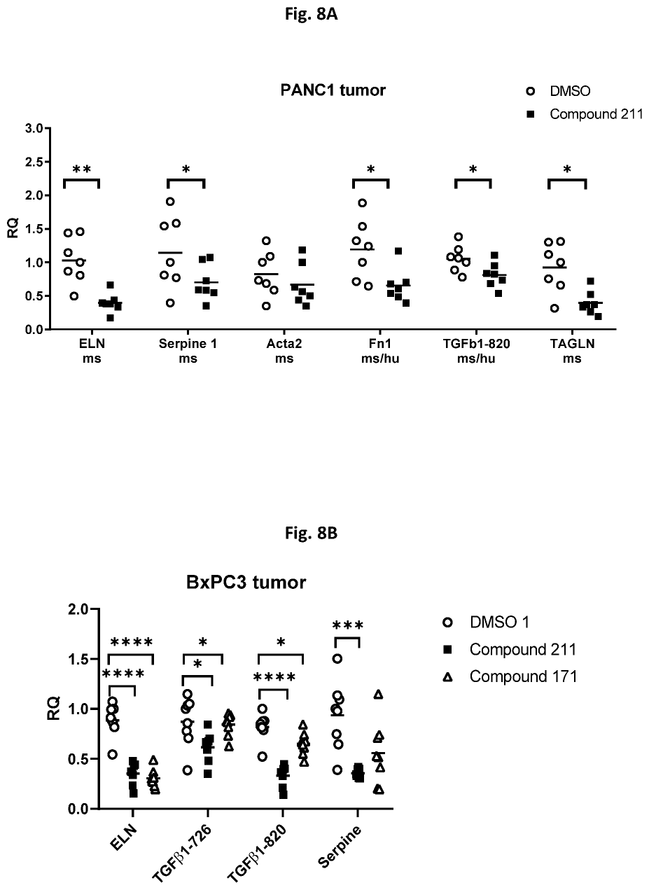

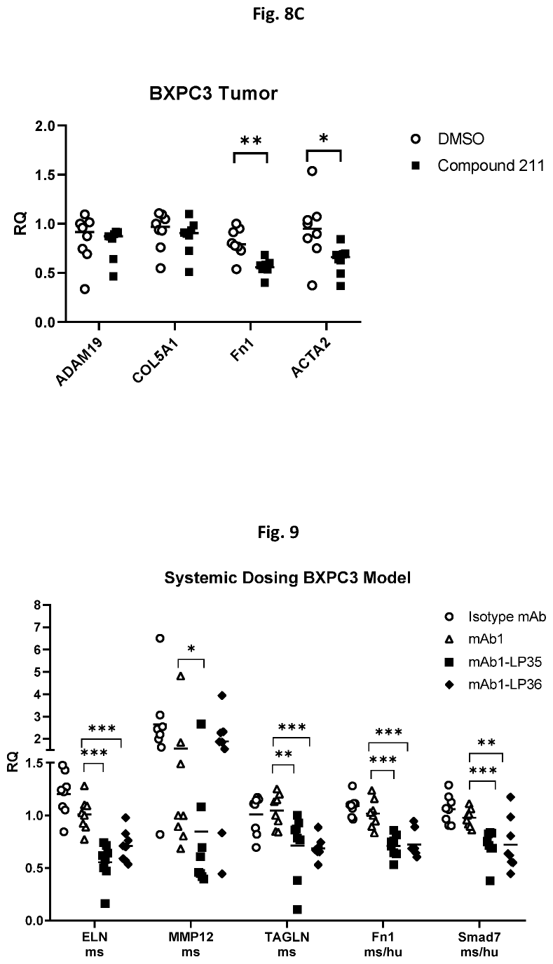

TGFβR2 inhibitors were tested for their ability to alter expression in vivo of a set of genes reported to be regulated by TGFβ, particularly those associated with tumor CAFs. Two million BXCP3 (ATTC) or 5 million PANC-1 (ATTC) cells in Matrigel (Corning Life Sciences) were inoculated into the flanks of nude mice (JAX Labs) and allowed to grow to approximately 100 mm3 in size before sorting into cohorts for treatment. BXPC3 tumors were treated by intra-tumoral injection (IT) of 2 ug QD5 of either compound 211, compound 171 or equal volume of DMSO vehicle while PANC-1 tumors received 2 ug injections QD5 of compound 211 or vehicle control. Four hours after the last injection tumors were removed and processed for RT-qPCR by homogenization in M tubes (Miltenyi Biotec) using Purelink RNA Kit (Ambion) to isolate RNA and Taqman® RNA to CT 1 Step Kit (Applied Biosystems) with a OneStepPlus Thermocycler. Gene specific primers were obtained from ThermoFisher Scientific. Some primers were specific for the mouse gene target (ms) while others would measure gene mRNA levels derived from both human tumor cells and mouse cells in the tumor (ms/hu). ALAS1 mRNA was not affected by treatment and was used as the reference gene. Data was analyzed and graphed using GraphPad Prism v8.1.0. Statistical difference for gene mRNA expression between TGFβR2 inhibitor and vehicle treated cohorts was determined using multiple T test comparison with discovery (FDR) by the two-stage linear step-up procedure of Benjamini, Krieger and Yekutieli, with Q=5%. Each row was analyzed individually, without assuming a consistent SD. As shown in FIG. 8A compound 211 treatment decreased many TGFβ-inducible gene mRNAs in the PANC-1 tumors values including those with q values that passed the FDR test (**q<0.005; * q<0.05). Also as shown in FIGS. 8B and 8C treatment with either compound 211 or compound 171 (B) and or compound 211 (C) reduced the mRNA level of most TGFβ-inducible genes in the BXPC3 tumors including some that passed the FDR test (* q<0.05, ** q<0.005*** q<0.0005, **** q<0.00005).

### Example 16: In Vivo Intra-Tumoral Injection of TGFβR2 Inhibitor Conjugates Reduces Gene Expression of TGFβ-Inducible Genes in the Tumor

To demonstrate that a TGFβR2 inhibitor conjugated to an anti-LRRC15 antibody with mouse IgG2a constant regions reduces tumor mRNAs for TGFβ-inducible genes in vivo, compound 171 was covalently linked to the antibody at reactive cysteine residues after by partial cystine reduction with a maleimide PABC cleavable linker to produce anti-LRRC15-LP35 at an average DAR of 3.9. PANC-1 tumor cells were injected into the flank of nude mice (5,000,000 in Matrigel) and allowed to grow to 100 mm³ before beginning treatment by intra-tumoral injection of either PBS vehicle, 157 ug of non-conjugated anti-LRRC15 antibody or 157 ug of the conjugate every other day for a total of 3 doses. Tumors were harvested eight hours after the last dose, processed and subjected to RT-qPCR, and analyzed as described above. Statistical difference for gene mRNA expression between TGFβR2 inhibitor and vehicle treated cohorts was determined using multiple T test comparison with discovery (FDR) by the two-stage linear step-up procedure of Benjamini, Krieger and Yekutieli, with Q=5%. Each row was analyzed individually, without assuming a consistent SD. The conjugate reduced the mean of mRNA of all the genes (ELN, TAGLN, Fnl, TGFb1 and Acta2) with a number passing the FDR test (data not shown).

### Example 17: Systemic Delivery of an Anti-LRRC15 Conjugates Reduce TGFβ Regulated Genes in a Mouse Human Tumor Xenograft Model

Anti-LRRC15-mIgG2a conjugates of two TGFβR2 inhibitors covalently attached to the LRRC15 antibody at cysteine residues by different linkers were tested for their ability to inhibit expression of TGFβ-regulated genes after systemic dosing. Nude mice (JAX Labs) had 1.75 million BXPC3 tumor cells seeded into a flank and were sorted into treatment cohorts after tumors reached approximately 100 mm³. Animals received an intravenous dose of 20 mpk for 4 consecutive days of either an anti-LRRC15-LP35 (wildtype IgG2), an anti-LRRC15-LP36 conjugate (Fc null IgG2), or controls consisting of the unconjugated anti-LRRC15 antibody or an isotype-matched control of the anti-LRRC15 antibody. Tumors were harvested 8 hours after the final dose, processed for RNA isolation and the RNAs subjected to RT-qPCR as described above. The results, shown in FIG. 9 were analyzed statistically as in Example 14 using a Q=1% comparing gene expression in animals treated with the unconjugated antibody with the conjugates. The FDR test was passed for comparisons marked with asterisks (* q<0.01, ** q<0.001, *** q<0.0001).

### Example 18: Systemic Delivery of an Anti-LRRC15 TGFβR2 Inhibitor Conjugate is Effective in Reducing TGFβ-Regulated Gene Expression in Tumors at Two Dose Levels

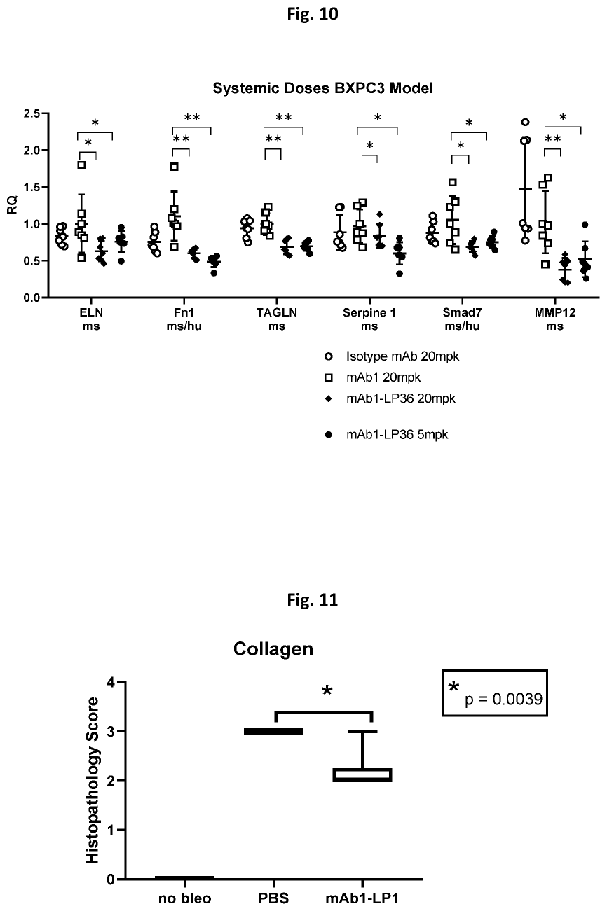

An anti-LRRC15 antibody TGFbR2 inhibitor conjugate, anti-LRRC15-LP36, was administered by intravenous route at 2 different dose levels into nude mice bearing a xenograft of the human tumor cell line BXPC3. Two million tumor cells per mouse were injected into the flank and after the tumors reached an approximate size of 100 mm³ were sorted into treatment groups. Group received 4 daily intravenous injections of either 20 mpk anti-LRRC15-LP36, 5 mpk anti-LRRC15-LP36, 20 mpk of unconjugated LRRC15 antibody or 20 mpk of an isotype control antibody. Eight hours post final dose tumors were harvested, RNA was isolated and RT-qPCR performed as described in Example 14. The results are shown in FIG. 10. When analyzed statistically by the multiple T test for discovery with a FDR of Q=5% all the genes were discovered (* q<0.05; ** q<0.005) at both doses showing effective lowered TGFβ-regulated gene expression compared to the unconjugated antibody.

### Example 19: Conjugate Activity Tracked Activity of Small Molecules

Anti-LRRC15 or anti-HER2 conjugates were prepared comprising compounds 55, 56, 57, 61, 62, 64, 65, 67, 70, 73, 75, 83, 87, 88, 91, 102, 108, 112, 127, 129, 132, 136, 138, 139, 140, 142, 145, 162, 168, 169, 170, 171, 175, 181, 182, 184, 190, 194, 195, 197, 202, 203, 205, 211, 213, 219, 220, 222, 224, and 256 attached to linker L1 below at a substitutable nitrogen as shown in Table 9B. Compound 171 was conjugated to Linkers L1, L2, L3 and L4 below. Average DAR was between 2 and 5.

TABLE 20

| | |
|---|---|
| Linker L1 | 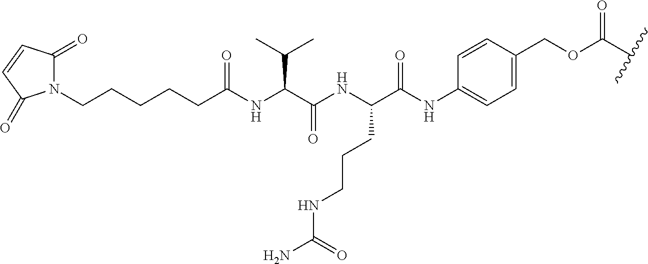 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1] |
| Linker L2 | 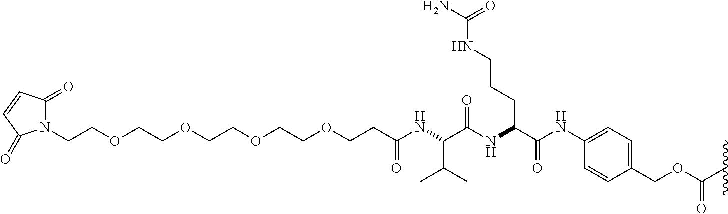 |
| Linker L3 | 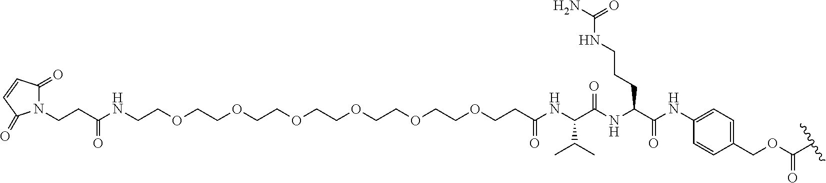 [SMILES: *C(=O)OCc1ccc(NC(=O)[C@H](CCCNC(N)=O)NC(=O)[C@@H](NC(=O)CCOCCOCCOCCOCCOCCOCCNC(=O)CCN2C(=O)C=CC2=O)C(C)C)cc1] |
| Linker L4 | 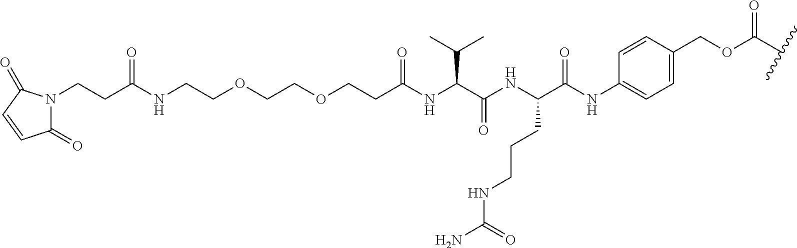 [SMILES: *C(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1] |

The potency of the described TGFβR2-antibody drug conjugates tested via the cell-based reporter assay track proportionally with the activity observed for the small molecule activity within the small molecule cell-based reporter assay. For examples in which the observed activity of a small molecule is low when assessed by the small molecule cell-based reporter assay and high by measure within the small molecule cell-free enzymatic inhibition assay, it is believed, without being bound by theory, that this can most often be attributed to the molecule possessing low cell permeability. In these cases, the rank order of potency of the applicable TGFβR2-antibody drug conjugate tracks more closely with the observed activity within the small molecule cell-free enzymatic inhibition assay.

### Example 20: TGFβR2 Inhibitors Conjugated to an Anti-LRRC15 Antibody Decrease Histopathological Fibrosis in a Model of Systemic Scleroderma

Fibrosis was elicited in the skin of 5 to 7 week old female C3H/HeJ mice by daily subcutaneous injection of bleomycin (0.1 ml of 1.2u/ml) for 23 days. Compound 2.1 was covalently attached to an anti-LRRC15 antibody (humanized M25 antibody with wild-type IgG1 Fc domain) using a protease cleavable PABC linker. The conjugate was administered 3xw ip at a dose of 10 mpk to a cohort of 10 animals beginning on day 14 and ending day 22. As a control, a matched cohort of mice received PBS vehicle only. On day 23, mice were sacrificed and skin from the injectate site was fixed with paraformaldehyde and stained with Masson's Trichrome Stain by standard procedures to reveal collagen. Fibrosis, as assessed by collagen, was scored by a histopathologist using the grading system set forth in Table 21. As seen in FIG. 11, treatment with the conjugate reduced fibrosis assessed by this measure reaching indicated statistical significance using Dunn's Multiple Comparison test.

TABLE 21

| | |
|---|---|
| 0 = Normal | Tissue Considered to be normal under the |
| | conditions of the study and considering the age, sex |
| | and strain of the animal concerned. Alterations may |
| | be present which, under other circumstances, |
| | would be considered deviations from normal |
| 1 = Minimal | The amount of change barely exceeds that which is |
| | considered to be within normal limits |
| 2 = Mild | In general, the lesion is easily identified but of |
| | limited severity. The lesion probably does not |
| | produce any functional impairment |
| 3 = Moderate | The lesion is prominent but there is significant |
| | potential for increased severity. Limited tissue or |
| | organ dysfunction is possible |
| 4 = Severe | The degree is either as complete as considered |
| | possible or great enough in intensity or extent to |
| | expect significant tissue or organ dysfunction |

### Example 21: TGFβR2 Inhibitors Conjugated to an Anti-LRRC15 Antibody Decrease Fibrosis in a Model of Systemic Fibrosis

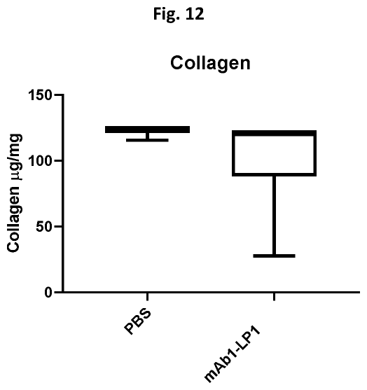

Compound 2.1 was covalently attached to an anti-LRRC15 antibody (humanized M25 antibody with wild-type IgG1 Fc domain) using a protease cleavable PABC linker. An independent assessment of fibrosis by collagen deposition in the conjugate treated animals was assayed biochemically by using a Sircol Collagen Assay on a weighed piece of non-fixed skin from the injectate site using the manufacturer's protocol to determine micrograms/mg of collagen. As seen in FIG. 12, when analyzed by one-way Anova, the treated cohort had significantly reduced fibrosis by this measure.

1. A compound represented by Formula (I): 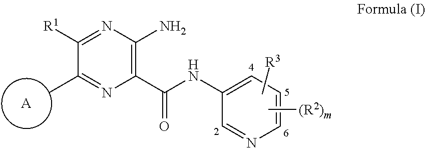
or a pharmaceutically acceptable salt thereof, wherein:
Ring A is unsubstituted or substituted cycloalkyl, unsubstituted or substituted heterocycloalkyl, unsubstituted or substituted aryl, or unsubstituted or substituted heteroaryl, wherein when Ring A is substituted, substituents on Ring A are independently selected at each occurrence from R⁴;
each R⁴ is selected from R^{L} and R²⁰, or two R⁴ on adjacent atoms are taken together with the atoms to which they are attached to form an unsubstituted or substituted monocyclic carbocycle or unsubstituted or substituted monocyclic heterocycle;
R^{L} is 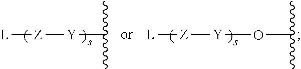
each Y is independently unsubstituted or substituted C₁-C₆alkylene; wherein when Y is substituted, substituents on Y are independently selected at each occurrence from R⁵;
each R⁵ is selected from R²⁰, or two R⁵ on adjacent atoms are taken together with the atoms to which they are attached to form an unsubstituted or substituted monocyclic carbocycle, or unsubstituted or substituted monocyclic heterocycle;
each Z is independently —NR⁶S(═O)₂—, —S(═O)₂NR⁶—, —OC(═O)—, —C(═O)O—, —C(═O)NR⁶—, or —NR⁶C(═O)—; wherein each R⁶ is independently selected from hydrogen, unsubstituted or substituted C₁-C₆alkyl, unsubstituted or substituted carbocycle, and unsubstituted or substituted heterocycle, or an R⁵ and an R⁶ on adjacent atoms are taken together with the atoms to which they are attached to form an unsubstituted or substituted monocyclic heterocycle;
L is unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; wherein when L is substituted, substituents on L are independently selected at each occurrence from R⁷;
each R⁷ is selected from —SSR⁵⁰ and R²⁰;
s is 1-10;
R¹ is selected from hydrogen and R²⁰;
each R² is independently selected from R²⁰, or two R² on adjacent atoms are taken together with the atoms to which they are attached to form an unsubstituted or substituted monocyclic carbocycle or unsubstituted or substituted monocyclic heterocycle;
m is 0-3;
R³ is selected from (i), (ii), (iii), and (iv):
(i) unsubstituted or substituted aryl, or unsubstituted or substituted heteroaryl; wherein when R³ is substituted, substituents on R³ are independently selected at each occurrence from R¹⁰;
(ii) unsubstituted or substituted cycloalkyl, or unsubstituted or substituted heterocycloalkyl; wherein when R³ is substituted, substituents on R³ are independently selected at each occurrence from R¹¹;
(iii) unsubstituted or substituted polycyclic heterocycloalkyl, unsubstituted or substituted 3- to 5-membered monocyclic heterocycloalkyl, unsubstituted or substituted 6- to 8-membered monocyclic heterocycloalkyl comprising 1 or 2 N atoms and 1 or 2 other heteroatoms selected from O or S; wherein when R³ is substituted, substituents on R³ are independently selected at each occurrence from R¹¹; and 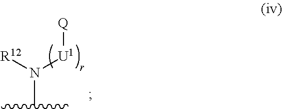
wherein when R³ is at the 2-, 5-, or 6-position of the pyridine, R³ is selected from (i), (ii), and (iv), and when R³ is at the 4-position of the pyridine, R³ is selected from (i), (iii), and (iv); and
each R¹⁰ is selected from R²⁰, or two R¹⁰ on adjacent atoms are taken together with the atoms to which they are attached to form an unsubstituted or substituted monocyclic carbocycle or unsubstituted or substituted monocyclic heterocycle;
each R¹¹ is selected from ═O, ═S, and R²⁰;
R¹² is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
Q is —OR¹³, —NR¹³R¹³, —SR¹³, —CN, —C(═O)R¹⁴, —C(═O)NR¹³R¹³, —S(═O)R¹⁴, or —S(═O)₂R¹⁴, or —S(═O)₂NR¹³R¹³;
R¹³ is hydrogen, unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
R¹⁴ is unsubstituted or substituted C₁-C₆alkyl, unsubstituted or substituted C₁-C₆alkenyl, unsubstituted or substituted C₁-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
each U¹ is —(CR¹⁵R¹⁶)—, wherein each R¹⁵ and R¹⁶ are independently selected from hydrogen and R²⁰;
r is 1-5;
each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
each R⁵⁰ is independently selected from unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
each R⁵¹ is independently selected from hydrogen, unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
or two R⁵¹ on the same N atom are taken together with the N atom to which they are attached to form an unsubstituted or substituted N-containing heterocycle;
wherein when any of R², R⁴, R⁵, R⁶, R¹⁰, R¹², R¹³, R¹⁴, R²⁰, R⁵⁰, and R⁵¹ are substituted, substituents on the R², R⁴, R⁵, R⁶, R¹⁰, R¹², R¹³, R¹⁴, R²⁰, R⁵⁰, and R⁵¹ are independently selected at each occurrence from halogen, —CN, —NO₂, —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², unsubstituted or substituted C₁-C₆alkyl, C₁-C₆haloalkyl, unsubstituted or substituted monocyclic carbocycle, unsubstituted or substituted monocyclic heterocycle, or two substituents on the same carbon atom are taken together to form a C═O or C═S and wherein substituents on said C₁-C₆alkyl are independently selected from R⁵⁴ and substituents on said carbocycle and heterocycle are independently selected from R⁵⁵;
each R⁵² is independently selected from hydrogen, C₁-C₆alkyl, C₃-C₆cycloalkyl, phenyl, benzyl, 5-membered heteroaryl, and 6-membered heteroaryl;
or two R⁵² groups are taken together with the N atom to which they are attached to form a N-containing heterocycle; and
each R⁵³ is independently selected from C₁-C₆alkyl, C₃-C₆cycloalkyl, phenyl, benzyl, 5-membered heteroaryl, and 6-membered heteroaryl.
each R⁵⁴ is independently selected from —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², and phenyl;
each R⁵⁵ is independently selected from —OR⁵², —CO₂R⁵², —C(═O)R⁵³, —C(═O)NR⁵²R⁵², —NR⁵²R⁵², —NR⁵²C(═O)R⁵³, —NR⁵²C(═O)OR⁵², and unsubstituted or substituted C₁-C₆alkyl wherein substituents on said C₁-C₆alkyl are independently selected from R⁵⁴.

2-13. (canceled)

14. The compound or salt of claim 1, wherein the compound of Formula (I) is represented by Formula (II): 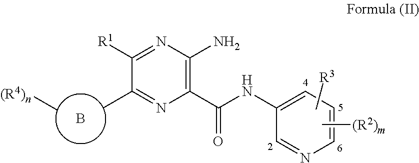
wherein:
ring B is aryl or heteroaryl; and
n is 0-5.

15-30. (canceled)

31. The compound or salt of claim 1, wherein each R⁴ is independently

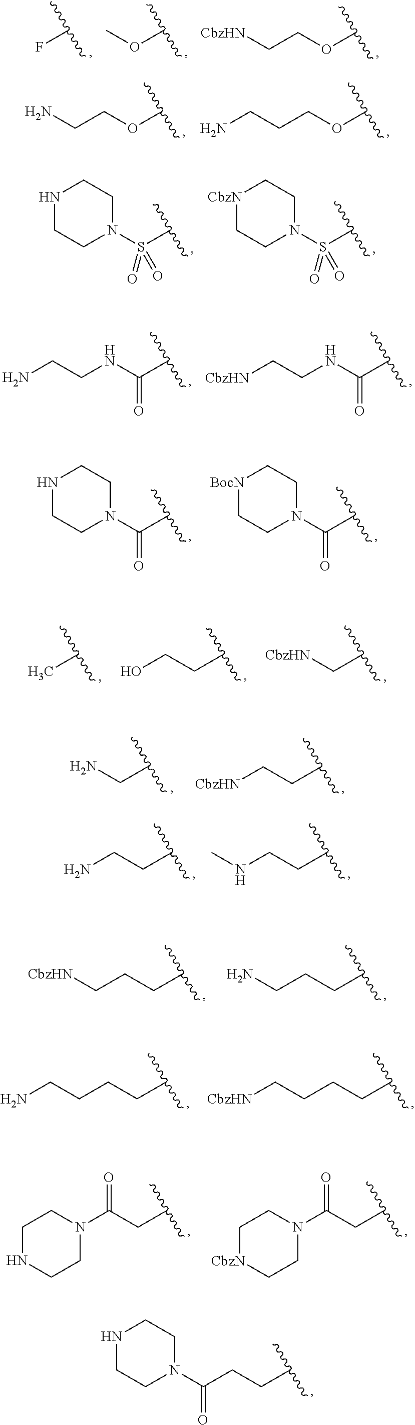

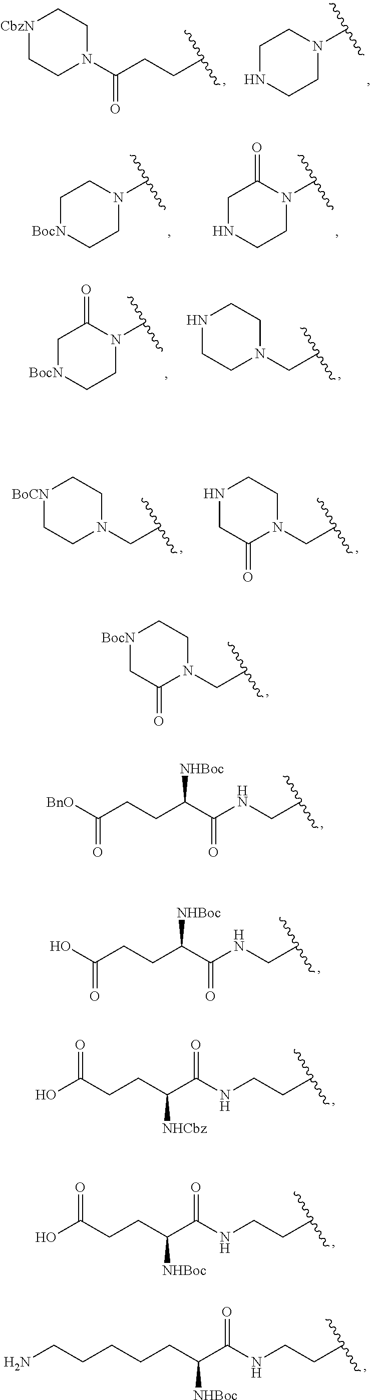

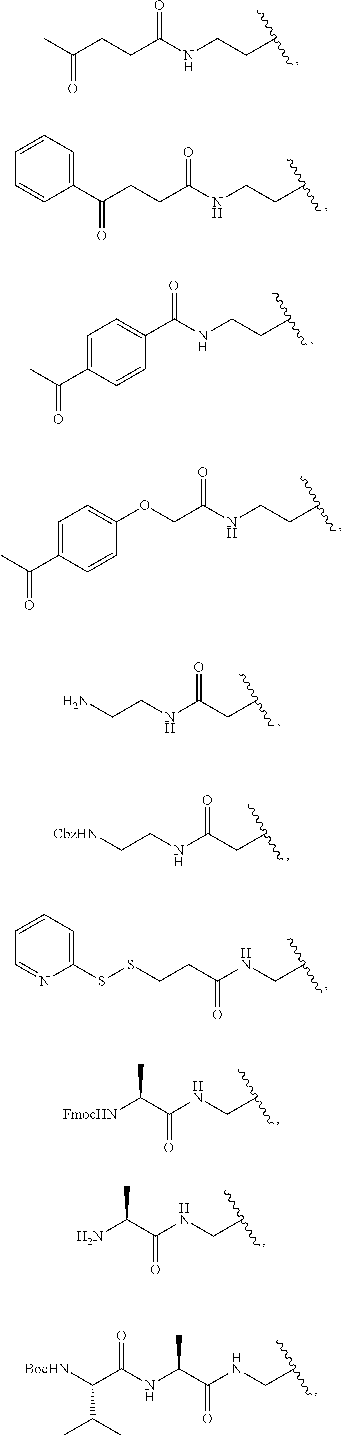

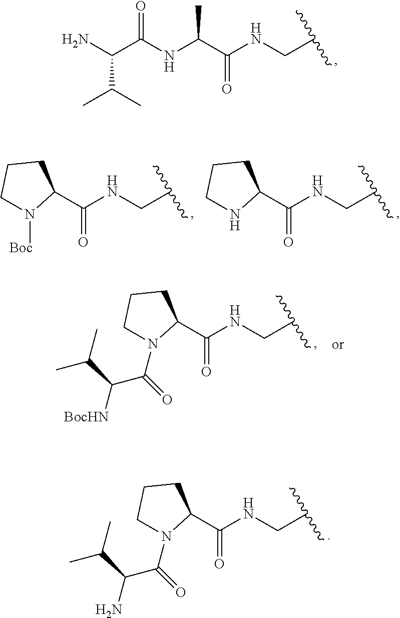

32-33. (canceled)

34. The compound or salt of claim 14, wherein

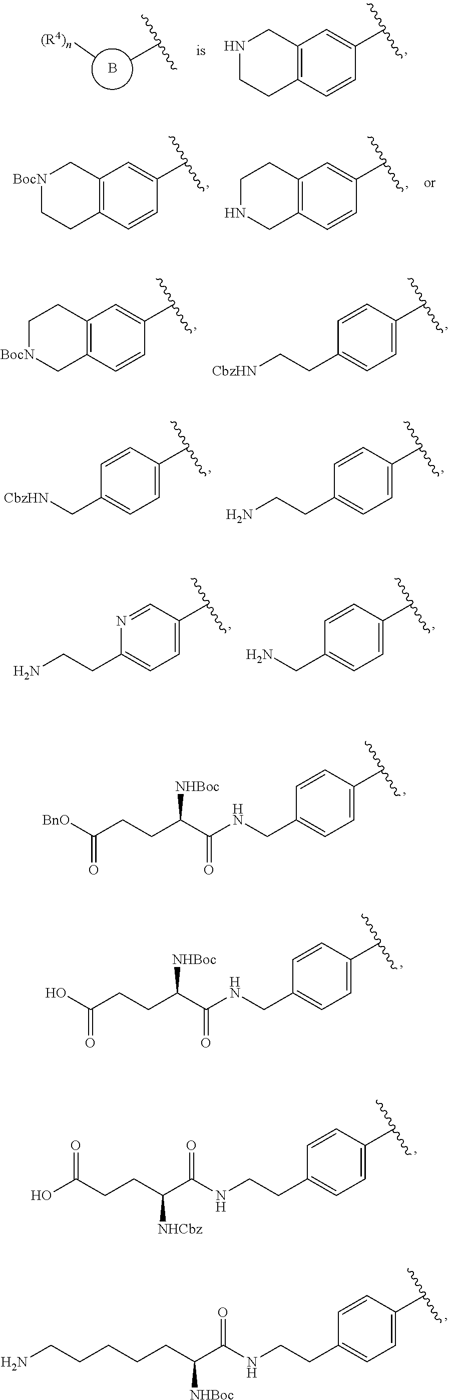

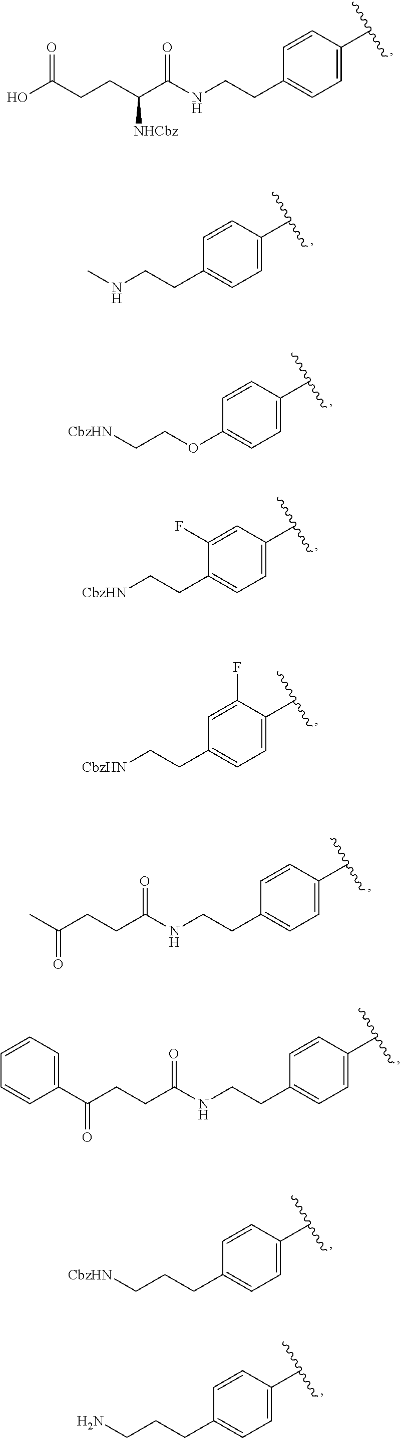

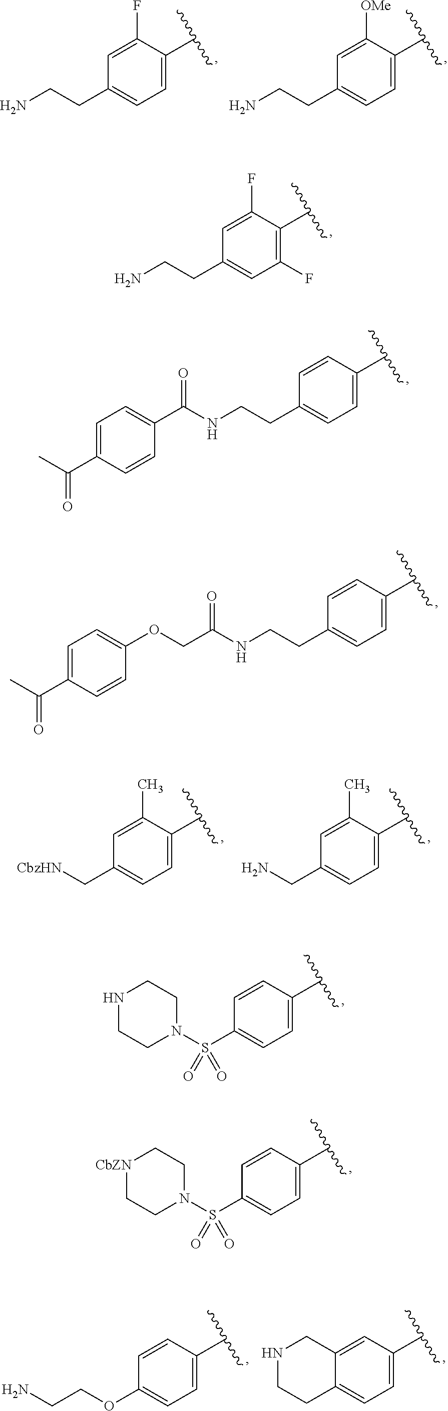

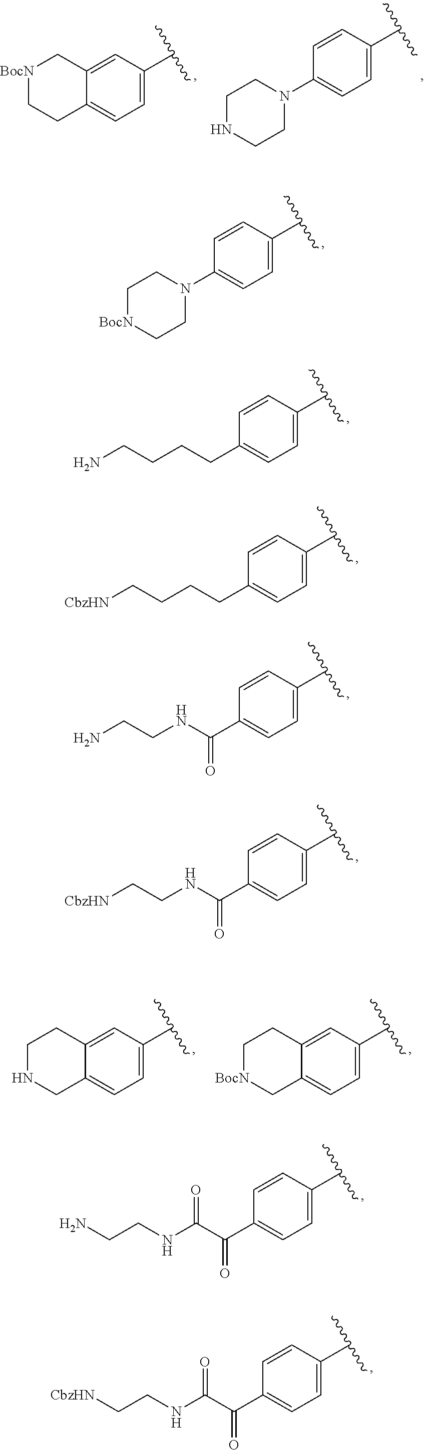

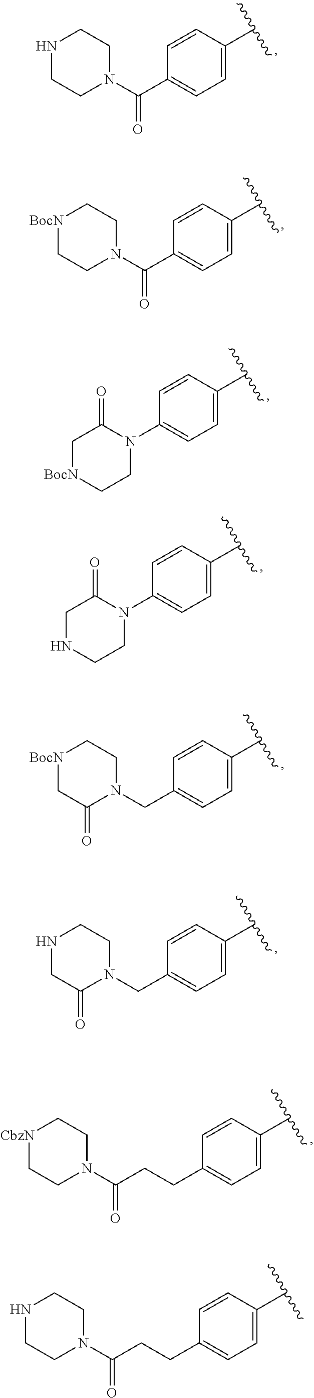

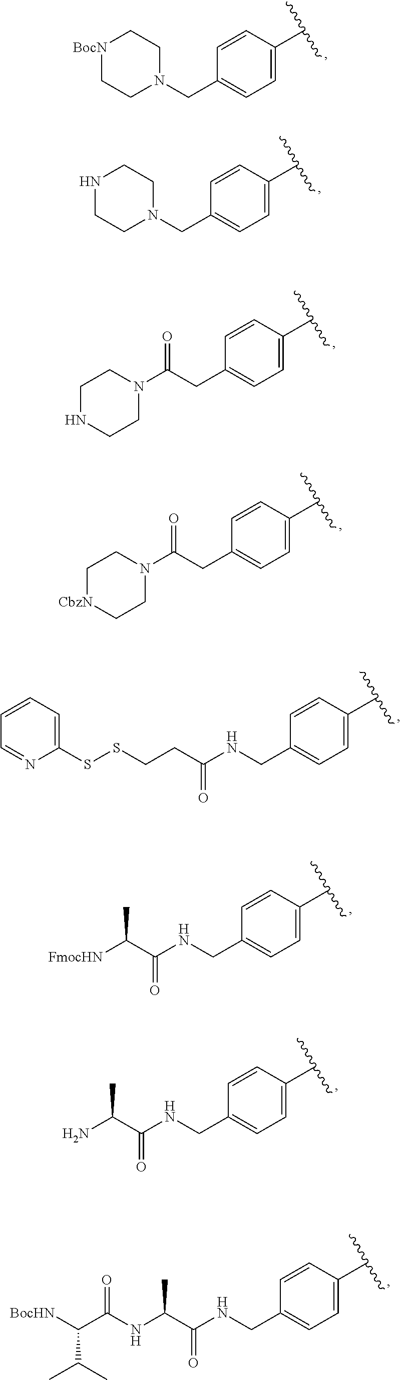

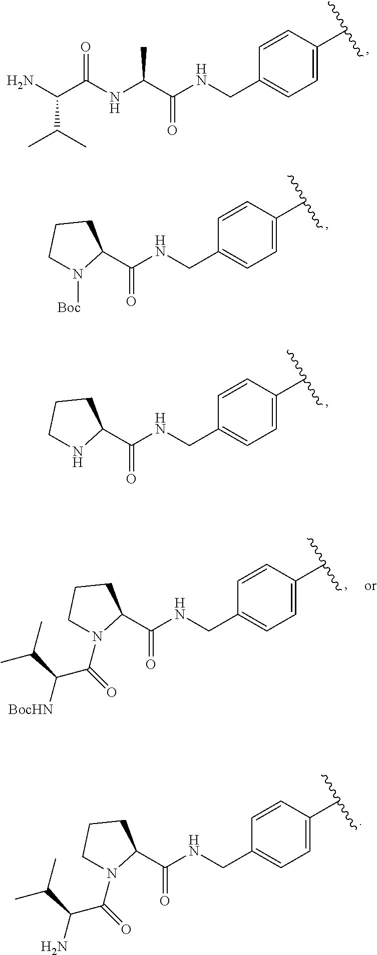 [SMILES: *c1ccc(CNC(=O)[C@H](C)NC(=O)[C@@H](N)C(C)C)cc1 ; *c1ccc(CNC(=O)[C@@H]2CCCN2C(=O)OC(C)(C)C)cc1 ; *c1ccc(CNC(=O)[C@@H]2CCCN2)cc1 ; *c1ccc(CNC(=O)[C@@H]2CCCN2C(=O)[C@@H](NC(=O)OC(C)(C)C)C(C)C)cc1 ; *c1ccc(CNC(=O)[C@@H]2CCCN2C(=O)[C@@H](N)C(C)C)cc1]

35. The compound or salt of claim 14, wherein:
each R⁴ is independently selected from halogen, —OH, —OR⁵⁰, —NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR¹C(═O)NR⁵¹R⁵¹, —NR⁵¹C(═O)R⁵⁰, —NR⁵¹C(═O)OR⁵¹, unsubstituted or substituted carbocycle, unsubstituted or substituted heterocycle, unsubstituted or substituted —C₁-C₆alkylene-carbocycle, unsubstituted or substituted —C₁-C₆alkylene-heterocycle and substituted C₁-C₆alkyl, with the proviso that C₁-C₆alkyl is substituted with —NR⁵²R⁵² and at least one of —OR⁵², —CO₂R⁵², —(C₁-C₆alkyl)-OR⁵², or (C₁-C₆alkyl)-CO₂R⁵²,
or two R⁴ on adjacent atoms are taken together with the atoms to which they are attached to form an unsubstituted or substituted monocyclic carbocycle or unsubstituted or substituted monocyclic heterocycle.

36-75. (canceled)

76. The compound or salt of claim 35, wherein at least one of R⁴ is:

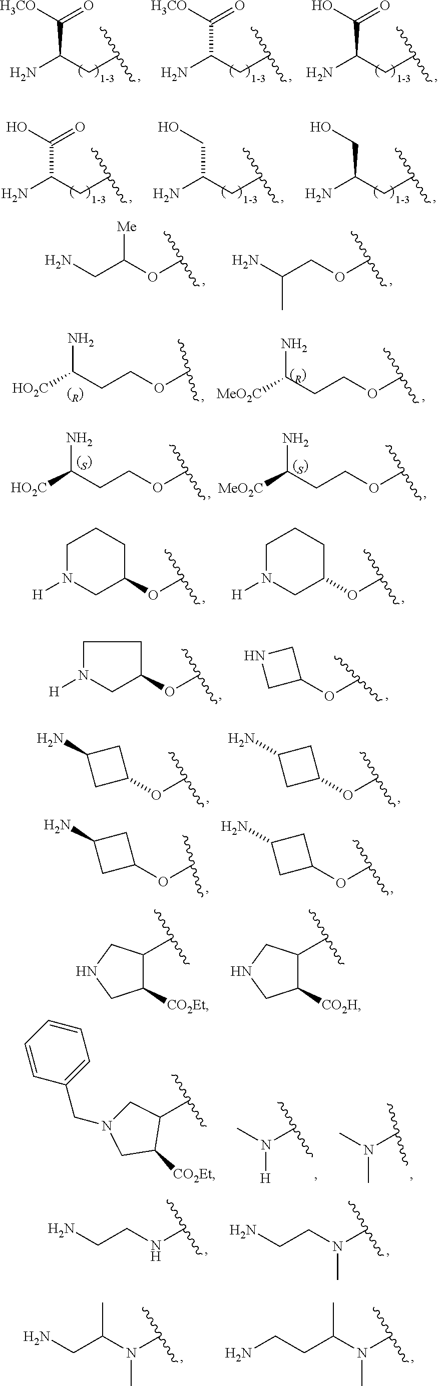

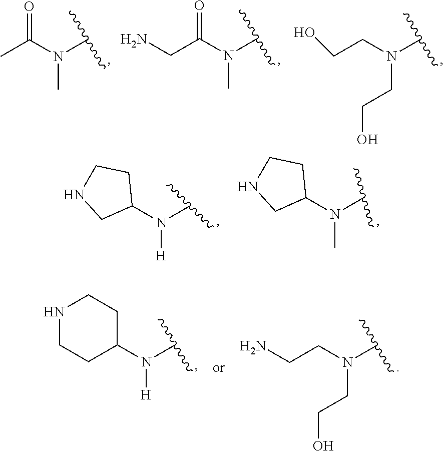

77. (canceled)

78. The compound or salt of claim 35, wherein

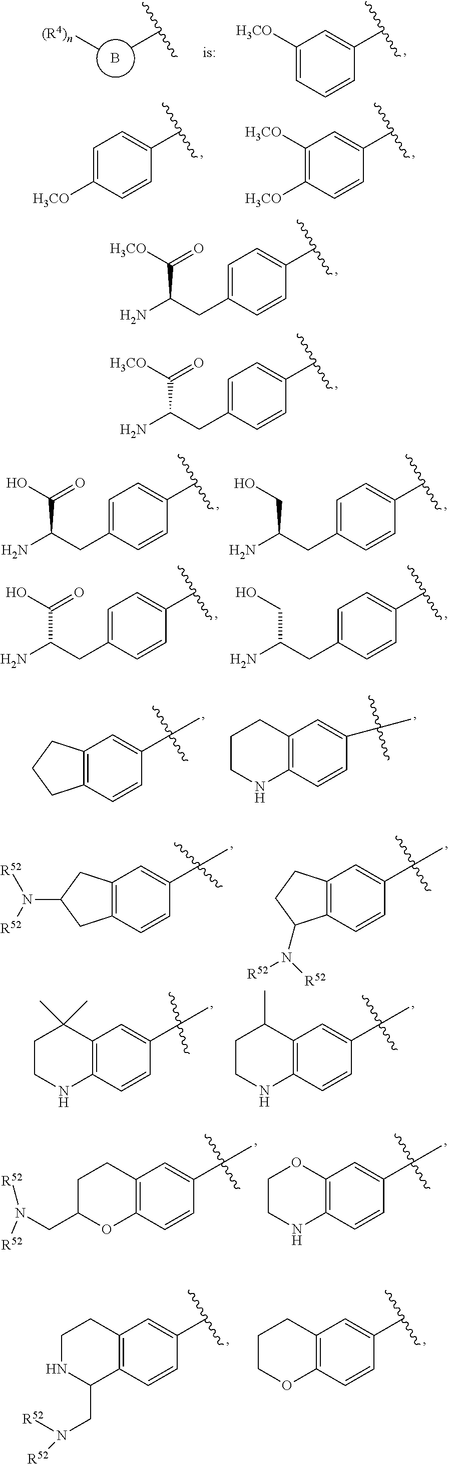

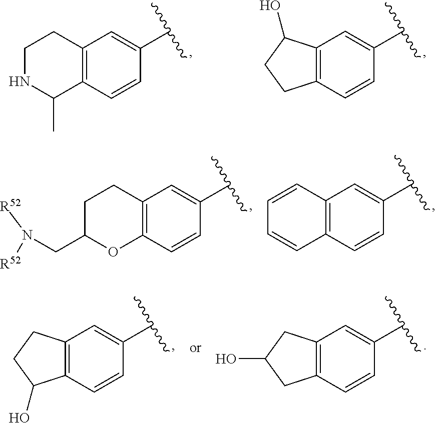

79. (canceled)

80. The compound or salt of claim 1, wherein the compound of Formula (I) is represented by Formula (I-A), Formula (I-B), or Formula (I-C):

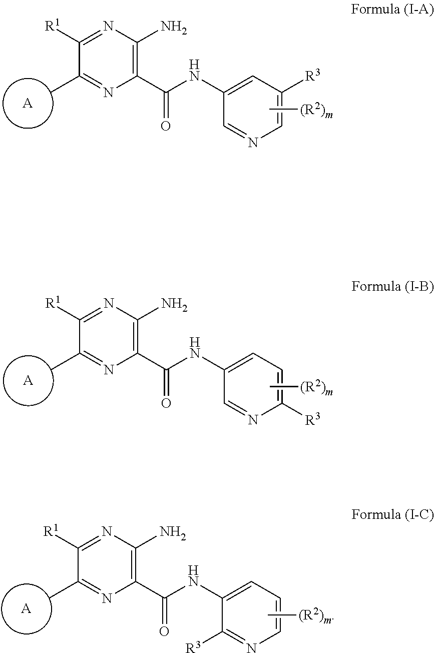

81. (canceled)

82. The compound or salt of claim 1, wherein the compound of Formula (I) is represented by Formula (I-D):

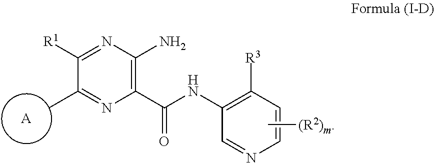

83. The compound or salt of claim 1, wherein the compound of Formula (I) is represented by Formula (II-A), (II-B), or (IIC).

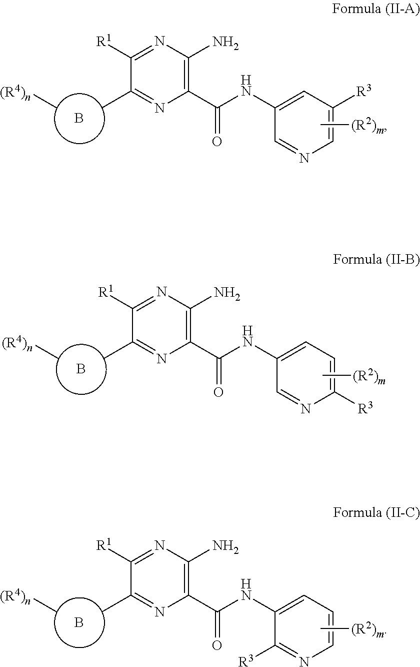

84. The compound or salt of claim 1, wherein the compound of Formula (I) is represented by Formula (II-D):

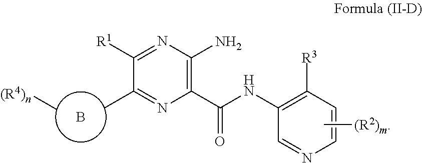

85-92. (canceled)

93. The compound or salt of claim 84, wherein R³

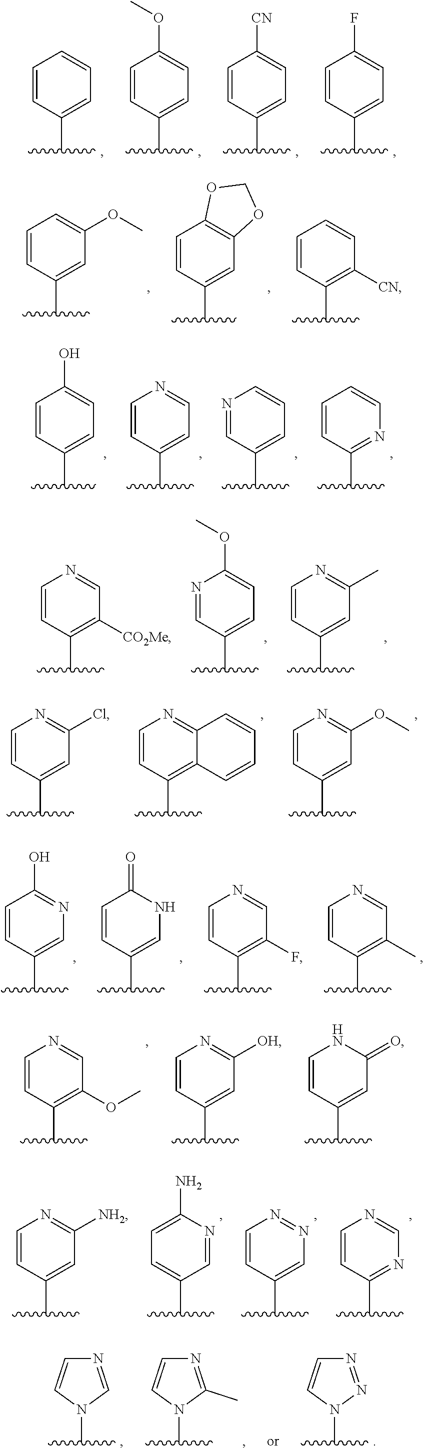

94-98. (canceled)

99. The compound or salt of claim 1, wherein R³ is

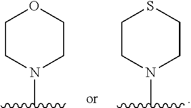 [SMILES: *N1CCOCC1 ; *N1CCSCC1]

100. The compound or salt of claim 99, wherein R³ is

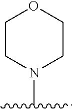 [SMILES: *N1CCOCC1]

101. The compound or salt of claim 1, wherein R³ is 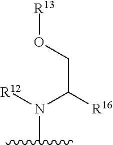
wherein R¹² and R¹⁶ are hydrogen and R¹³ is methyl.

102. The compound or salt of claim 1, wherein R³ is

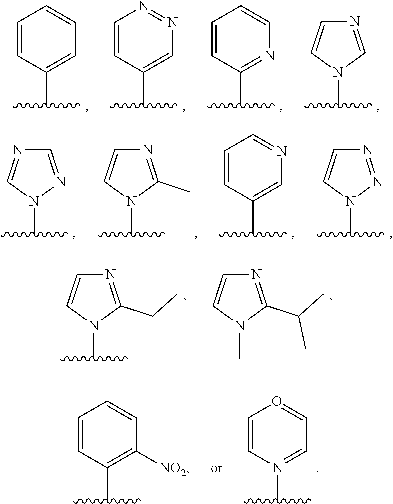

103. The compound or salt of claim 1, wherein
(a) Ring A is not substituted with —CH₂CH₂OH, or
(b) Ring A is not substituted with hydroxyalkyl, or
(c) Ring A is phenyl, said Ring is not substituted with —CH₂CH₂OH at the para position to the pyrazine, or
(d) Ring A is phenyl, Ring A is not substituted with —CH₂CH₂OH, or
(e) Ring A is phenyl, Ring A is not substituted with hydroxyalkyl, or
(f) Ring A is not substituted with a group selected from —CH₂CH₂NH₂, —CH₂NHBoc, —CH₂NH₂, 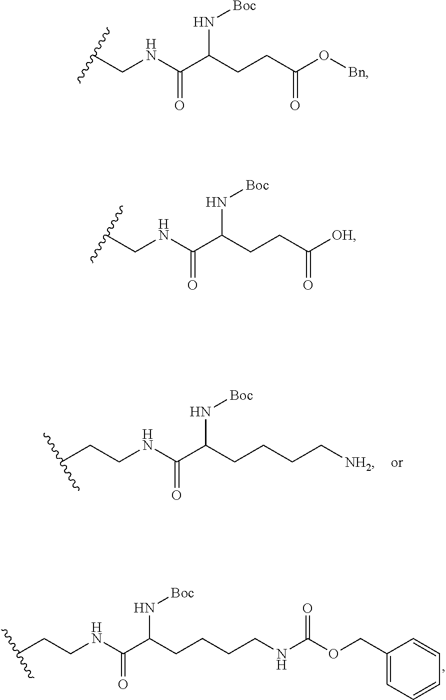 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; *CCNC(=O)C(CCCCNC(=O)OCc1ccccc1)NC(=O)OC(C)(C)C]
or
(g) Ring A is not substituted with an optionally substituted aminoalkyl group; or
(h) when Ring A is phenyl, Ring A is not substituted at the para position to the pyrazine with —CH₂CH₂NH₂, —CH₂NHBoc, —CH₂NH₂, 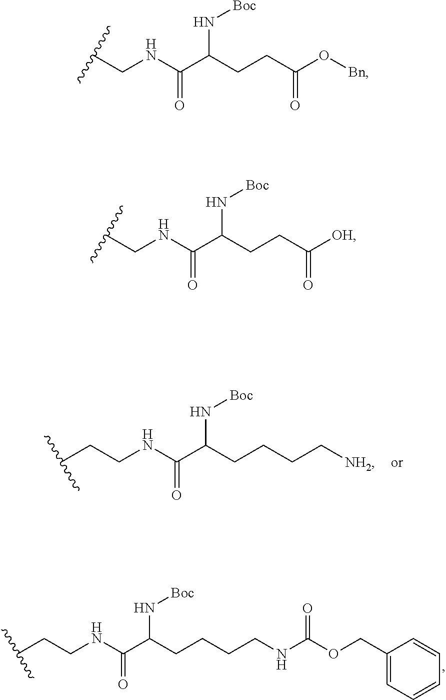 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; *CCNC(=O)C(CCCCNC(=O)OCc1ccccc1)NC(=O)OC(C)(C)C]
or
(i) when Ring A is phenyl, Ring A is not substituted with —CH₂CH₂NH₂, —CH₂NHBoc, —CH₂NH₂, 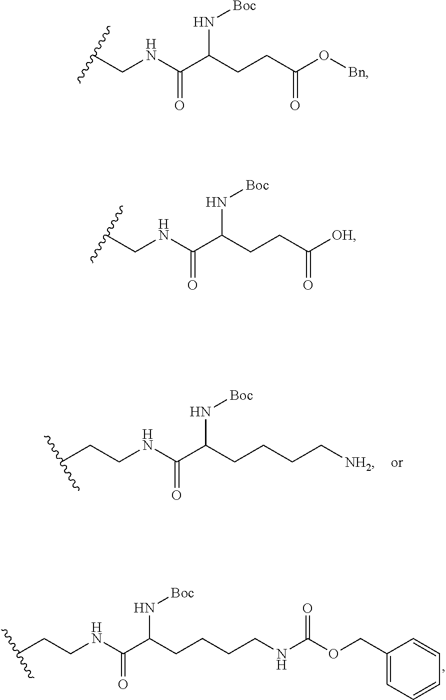 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; *CCNC(=O)C(CCCCNC(=O)OCc1ccccc1)NC(=O)OC(C)(C)C]
or
(j) when Ring A is phenyl, Ring A is not substituted with an optionally substituted aminoalkyl.

104-107. (canceled)

108. The compound or salt of claim 1, wherein the compound is not 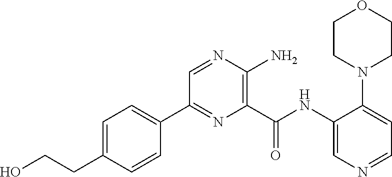 [SMILES: Nc1ncc(-c2ccc(CCO)cc2)nc1C(=O)Nc1cnccc1N1CCOCC1]
or a salt thereof.

109-113. (canceled)

114. The compound of claim 1, wherein the compound is not 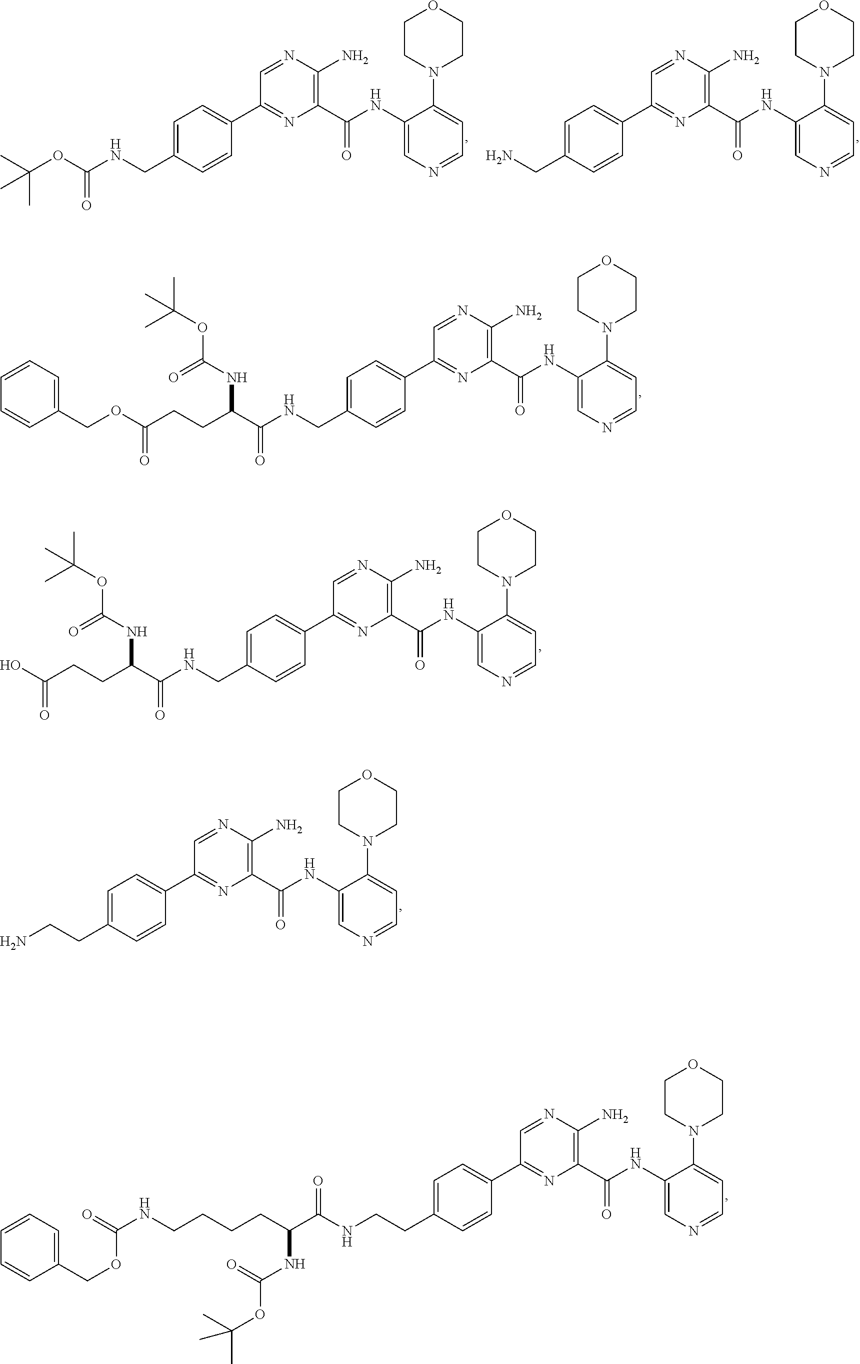 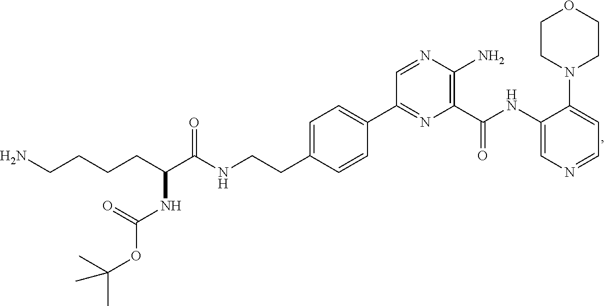 [SMILES: CC(C)(C)OC(=O)N[C@@H](CCCCN)C(=O)NCCc1ccc(-c2cnc(N)c(C(=O)Nc3cnccc3N3CCOCC3)n2)cc1]
or a salt of any one thereof.

115. A compound represented by Formula (I-E): 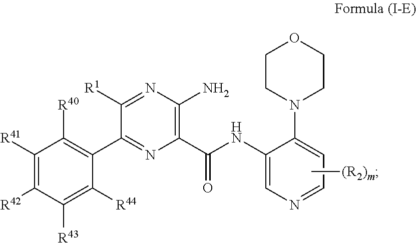
or a salt thereof, wherein:
each of R⁴⁰, R⁴¹, R⁴², R⁴³ and R⁴⁴ are independently selected from hydrogen, R^{L} and R²⁰; or two of R⁴⁰, R⁴¹, R⁴², R⁴³ and R⁴⁴ on adjacent atoms are taken together with the atoms to which they are attached to form an unsubstituted or substituted monocyclic carbocycle or unsubstituted or substituted monocyclic heterocycle wherein when said monocyclic carbocycle or said monocyclic heterocycle are substituted, substituents are independently selected at each occurrence from halogen, —CN, —NO₂, —OR⁵², —CO₂R⁵², —C(═O)R⁵³, —C(═O)N²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², C₁-C₆alkyl, C₁-C₆haloalkyl, aminoC₁-C₆alkyl-, Boc-aminoC₁-C₆alkyl-, Cbz-aminoC₁-C₆alkyl-, monocyclic carbocycle, and monocyclic heterocycle; or two substituents on the same carbon atom are taken together to form ═O or ═S;
each R⁵² is independently selected from hydrogen, C₁-C₆alkyl, C₃-C₆cycloalkyl, phenyl, benzyl, 5-membered heteroaryl, and 6-membered heteroaryl;
or two R⁵² groups are taken together with the N atom to which they are attached to form a N-containing heterocycle; and
each R⁵³ is independently selected from C₁-C₆alkyl, C₃-C₆cycloalkyl, phenyl, benzyl, 5-membered heteroaryl, and 6-membered heteroaryl;
R^{L} is 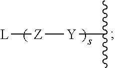
each Y is independently unsubstituted or substituted C₁-C₆alkylene; wherein when Y is substituted, substituents on Y are independently selected at each occurrence from R⁵;
each R⁵ is selected from R²⁰, or two R⁵ on adjacent atoms are taken together with the atoms to which they are attached to form an unsubstituted or substituted monocyclic carbocycle, or unsubstituted or substituted monocyclic heterocycle;
each Z is independently —NR⁶S(═O)₂—, —S(═O)₂NR⁶—, —OC(═O)—, —C(═O)O—, —C(═O)NR⁶—, or —NR⁶C(═O)—; wherein each R⁶ is independently selected from hydrogen, unsubstituted or substituted C₁-C₆alkyl, unsubstituted or substituted carbocycle, and unsubstituted or substituted heterocycle, or an R⁵ and an R⁶ on adjacent atoms are taken together with the atoms to which they are attached to form an unsubstituted or substituted monocyclic heterocycle;
L is unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle; wherein when L is substituted, substituents on L are independently selected at each occurrence from R⁷;
each R⁷ is selected from —SSR⁵⁰ and R²⁰;
s is 1-10;
R¹ is selected from hydrogen and R²⁰;
each R² is independently selected from R²⁰, or two R² on adjacent atoms are taken together with the atoms to which they are attached to form an unsubstituted or substituted monocyclic carbocycle or unsubstituted or substituted monocyclic heterocycle;
m is 0-3;
each R²⁰ is independently halogen, —CN, —OH, —OR⁵⁰, —SH, —SR⁵⁰, —NO₂, —NR⁵¹R⁵¹, —S(═O)₂R⁵⁰, —NR⁵¹S(═O)₂R⁵⁰, —S(═O)R⁵⁰, —S(═O)₂NR⁵¹R⁵¹, —C(═O)R⁵⁰, —OC(═O)R⁵⁰, —C(═O)OR⁵¹, —OC(═O)OR⁵¹, —C(═O)NR⁵¹R⁵¹, —OC(═O)NR⁵¹R⁵¹, —NR⁵¹C(═O)NR⁵¹R⁵¹, —NR⁵¹C(═O)R⁵⁰, —NR⁵¹C(═O)OR⁵¹, unsubstituted or substituted C₁-C₆alkyl, unsubstituted or substituted C₂-C₆alkenyl, unsubstituted or substituted C₂-C₆alkynyl, unsubstituted or substituted carbocycle, unsubstituted or substituted heterocycle, unsubstituted or substituted —C₁-C₆alkylene-carbocycle, or unsubstituted or substituted —C₁-C₆alkylene-heterocycle;
each R⁵⁰ is independently selected from unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle; and
each R⁵¹ is independently selected from hydrogen, unsubstituted or substituted C₁-C₆alkyl, unsubstituted or substituted carbocycle, unsubstituted or substituted heterocycle, unsubstituted or substituted —C₁-C₆alkylene-carbocycle, and unsubstituted or substituted —C₁-C₆alkylene-heterocycle;
or two R⁵¹ on the same N atom are taken together with the N atom to which they are attached to form an unsubstituted or substituted N-containing heterocycle;
wherein when any of R², R⁵, R⁶, R²⁰, R⁵⁰, and R⁵¹ are substituted, substituents on the of R², R⁵, R⁶, R²⁰, R⁵⁰, and R⁵¹ are independently selected at each occurrence from halogen, —CN, —NO₂, —OR⁵², —CO₂R⁵², —C(═O)R⁵³, —C(═O)N⁵²R⁵², —NR⁵²R⁵², —NR⁵²C(═O)R⁵³, —NR⁵²C(═O)OR⁵², —SR⁵², —S(═O)R⁵³, —SO₂R⁵³, —SO₂NR⁵²R⁵², C₁-C₆alkyl, C₁-C₆haloalkyl, monocyclic carbocycle, and monocyclic heterocycle; or two substituents on the same carbon atom are taken together to form a C═O or C═S; and
wherein R⁴² is not —CH₂CH₂OH, —CH₂CH₂NH₂, —CH₂NHBoc, —CH₂NH₂,

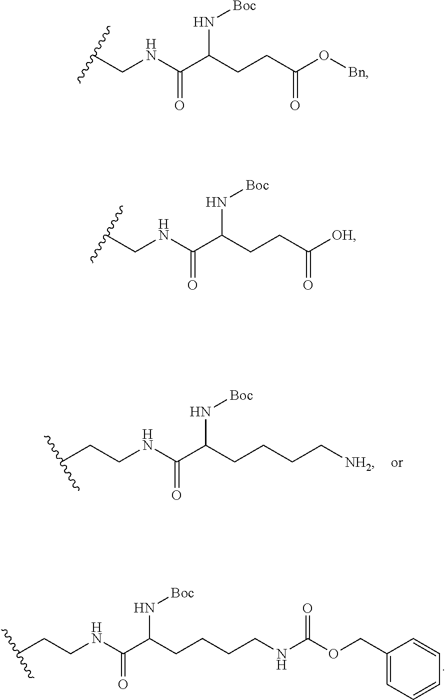 [SMILES: *CNC(=O)C(CCC(=O)OCc1ccccc1)NC(=O)OC(C)(C)C ; *CNC(=O)C(CCC(=O)O)NC(=O)OC(C)(C)C ; *CCNC(=O)C(CCCCN)NC(=O)OC(C)(C)C ; *CCNC(=O)C(CCCCNC(=O)OCc1ccccc1)NC(=O)OC(C)(C)C]

116-129. (canceled)

130. The compound or salt of claim 1, wherein the compound is selected from any one of compounds 1.1, 1.2, 2.1, 3.1, 4.1, 5.1 or 6 to 301 or a pharmaceutically acceptable salt of any one thereof.

131. A pharmaceutical composition, comprising a compound of claim 1 and a pharmaceutically acceptable excipient.

132. The compound or salt of claim 1, wherein the compound is covalently bound to a linker, L³ to form a compound-linker (compound-L³).

133. The compound-linker of claim 132, wherein (a) -L³ is covalently bound to a substitutable nitrogen atom, oxygen atom, or sulfur atom of the compound or salt, or (b) -L³ is a cleavable linker, or (c) -L³ is covalently bound to the compound or salt through Ring A or Ring B or (d) -L³ is selected from any one of the linkers set forth in Table 3, Table 4, Table 6, and Table 7; or a salt thereof, or (e) -L³ is represented by the formula: 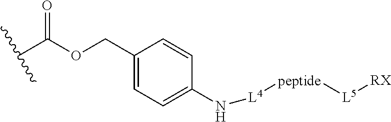
wherein:
L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and
R³⁰ is independently selected at each occurrence from halogen, —OH, —CN, —O— alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

134-145. (canceled)

146. The compound-linker of claim 133, wherein the peptide of -L³ comprises Val-Cit or Val-Ala.

147-155. (canceled)

156. The compound-linker of claim 132, wherein -L³ is further covalently bound to an antibody construct to form a conjugate.

157. A conjugate represented by the formula: 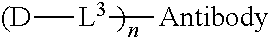
wherein:
Antibody is an antibody construct;
n is 1-20;
D is the compound or pharmaceutically acceptable salt of claim 1; and
L³ is a linker moiety.

158. (canceled)

159. The conjugate of claim 157, wherein -L³- is represented by the formula: 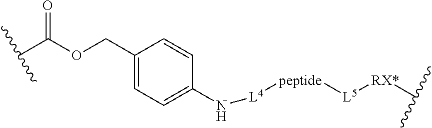
wherein:
L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to a residue of an antibody construct, wherein [symbol] on RX* represents the point of attachment to the residue of the antibody construct; and
R³⁰ is independently selected at each occurrence from halogen, —OH, —CN, —O— alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

160. The conjugate of claim 159, wherein RX* is a succinamide moiety, hydrolyzed succinamide moiety or a mixture thereof and is bound to a cysteine residue of an antibody construct.

161-163. (canceled)

164. The conjugate of claim 157, wherein the antibody construct comprises an antigen binding domain that specifically binds to a tumor antigen or an antigen associated with the pathogenesis of fibrosis.

165. The conjugate of claim 157 wherein the antibody construct comprises an antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis.

166. The conjugate of claim 157, wherein the antibody construct comprises
(a) an antigen binding domain that specifically binds to an antigen selected from the group consisting of CTLA-4, PD-1, OX40, LAG-3, GITR, GARP, CD25, CD27, PD-L1, TNFR2, ICOS, 41BB, CD70, CD73, CD38, and VTCN1, or
(b) an antigen binding domain that specifically binds to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11 and F2RL2, or
(c) an antigen binding domain that specifically binds to the LRRC15 antigen, or
(d) an antigen binding domain that specifically binds to an antigen on a hepatocyte, wherein the antigen is selected from the group consisting of ASGR1 and ASGR2.

167-175. (canceled)

176. A pharmaceutical composition comprising a conjugate of claim 157 and a pharmaceutically acceptable excipient.

177. The pharmaceutical composition of claim 176, wherein the average Drug-to-Antibody Ratio (DAR) is 1-8, 3-5, or 1-3.

178-179. (canceled)

180. A method for the treatment of cancer, comprising administering an effective amount of the pharmaceutical composition of claim 176 to a subject in need thereof.

181-186. (canceled)

187. A method for the treatment of fibrosis, comprising administering an effective amount of the pharmaceutical composition of claim 176 to a subject in need thereof.

188. The method of claim 187, wherein
(a) the fibrosis is associated with cancer, or
(b) the fibrosis is systemic fibrosis, or
(c) the fibrosis is scleroderma, or
(d) the fibrosis is associated with NASH.

189-208. (canceled)

209. A method for the treatment of cancer, comprising administering an effective amount of an antibody conjugate, wherein the antibody conjugate comprises (i) an antibody construct covalently bound through a linker group to a TGFbR2 antagonist, and wherein the antibody conjugate comprises from 1 to 20 TGFbR2 antagonists per antibody construct, or (ii) an antibody construct covalently bound through a linker group to a TGFbR2 antagonist, wherein the TGFbR2 antagonist comprises an amino-pyrazinecarboxamide, and wherein the antibody conjugate comprises from 1 to 20 TGFbR2 antagonists per antibody construct.

210-212. (canceled)
